(19) United States
(12) Patent Application Publication
(10) Pub. No.: US 2024/0343727 A1
(43) Pub. Date: Oct. 17, 2024

(54) PYRIDAZINONE OR PYRIDINONE COMPOUNDS, PREPARATION METHODS AND USES THEREOF

(71) Applicants: InventisBio Co., Ltd., Shanghai (CN); InventisBio LLC, Short Hills, NJ (US)

(72) Inventors: Xianhai HUANG, Florham Park, NJ (US); Younong YU, Florham Park, NJ (US); Haotao NIU, Shanghai (CN); Hong YANG, Shanghai (CN); Shanshan WANG, Shanghai (CN); Jifang WENG, Shanghai (CN); Yaolin WANG, Shanghai (CN); Xing DAI, Shanghai (CN)

(21) Appl. No.: 18/578,677

(22) PCT Filed: Aug. 16, 2022
(86) PCT No.: PCT/CN2022/112674

§ 371 Date: Jan. 11, 2024

(30) Foreign Application Priority Data
Aug. 17, 2021 (WO) PCT/CN2021/112906

(51) Int. Cl. C07D 471/04; A61K 31/501; A61K 31/502; A61K 31/5025; A61K 31/506; A61K 31/5377; A61P 35/00; C07D 401/14; C07D 403/14; C07D 413/14; C07D 417/14; C07D 487/04; C07D 487/10; C07D 491/052; C07D 491/107

(52) U.S. Cl. CPC C07D 471/04; A61K 31/501; A61K 31/502; A61K 31/5025; A61K 31/506; A61K 31/5377; A61P 35/00; C07D 401/14; C07D 403/14; C07D 413/14; C07D 417/14; C07D 487/04; C07D 487/10; C07D 491/052; C07D 491/107

(57) ABSTRACT

Provided herein are novel compounds, for example, compounds having a Formula (I), or a pharmaceutically acceptable salt thereof and pharmaceutical compositions comprising the same. Also provided herein are methods of preparing the compounds and methods of using the compounds, for example, in treating cancer.

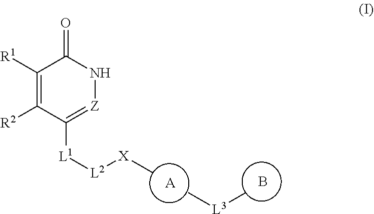

### CROSS REFERENCE TO RELATED APPLICATION

This application claims priority to International Application No. PCT/CN2021/112906, filed on Aug. 17, 2021, the content of which is incorporated herein by reference in its entirety.

### BACKGROUND

### Field of the Disclosure

The present disclosure is generally related to pyridazinone or pyridinone compounds, compositions, synthesis, and methods of use, for example, for treating various diseases or disorders herein, such as cancer.

### Background

Poly (ADP-ribose) polymerases (PARPs) are members of a family of 17 enzymes that regulate fundamental cellular processes. PARP1 inhibitors have been shown to be effective in treating cancers in connection to cellular stress induced by DNA damage. There are currently at least four approved PARP1 inhibitors and several others in late stage development. PARP7 (TIPARP; ARTD14) is a mono-ADP-ribosyltransferase involved in cellular processes such as responses to hypoxia, innate immunity and regulation of nuclear receptors. (Rasmussen, M. et al., Cells 10 (3): 623). PARP7 inhibition has been recently recognized as a strategy for cancer treatments and improving immunotherapy. Thus, there is a need for new PARP inhibitors, for example, for treating various associated diseases or disorders.

### BRIEF SUMMARY

In various embodiments, the present disclosure provides novel compounds, pharmaceutical compositions, and methods of preparing and using the same. Typically, the compounds herein are PARP inhibitors, in particular, PARP7 inhibitors. The compounds and compositions herein are also useful for treating various diseases or disorders herein, such as cancer.

In some embodiments, the present disclosure provides a compound of Formula I, or a pharmaceutically acceptable salt thereof,

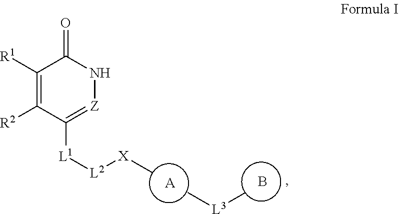

wherein the variables R¹, R², L¹, L², L³, X, Z, ring A, and ring B are defined herein. In some embodiments, the compound of Formula I can be characterized as having a subformula of Formula I, such as Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, as defined herein. In some embodiments, the present disclosure also provides a compound according to any of the compounds disclosed in Table A herein or a pharmaceutically acceptable salt thereof. In some embodiments, the present disclosure also provides a compound selected from Compound Nos. 1-353, or a pharmaceutically acceptable salt thereof.

Certain embodiments of the present disclosure are directed to a pharmaceutical composition comprising one or more of the compounds of the present disclosure (e.g., a compound of Formula I (e.g., I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, or a pharmaceutically acceptable salt thereof) and optionally a pharmaceutically acceptable excipient. The pharmaceutical composition described herein can be formulated for various routes of administration, such as oral administration, parenteral administration, or inhalation etc.

Certain embodiments are directed to a method of treating a disease or disorder associated with PARPs, in particular, PARP7. In some embodiments, the method comprises administering to a subject in need thereof a therapeutically effective amount of a compound of the present disclosure (e.g., a compound of Formula I (e.g., 1-1, 1-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, or a pharmaceutically acceptable salt thereof) or a therapeutically effective amount of a pharmaceutical composition described herein. Diseases or disorders associated with PARP7 suitable to be treated with the method include those described herein.

In some embodiments, a method of treating cancer is provided. In some embodiments, the method comprises administering to a subject in need thereof a therapeutically effective amount of a compound of the present disclosure (e.g., a compound of Formula I (e.g., 1-1, 1-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, or a pharmaceutically acceptable salt thereof) or a therapeutically effective amount of a pharmaceutical composition described herein. In some embodiments, the cancer can be breast cancer, cancer of the central nervous system, endometrium cancer, kidney cancer, large intestine cancer, lung cancer, esophagus cancer, ovarian cancer, pancreatic cancer, prostate cancer, stomach cancer, head and neck cancer (upper aerodigestive cancer), urinary tract cancer, or colon cancer. In some embodiments, the cancer is associated with abnormal PARP7 expression and/or activity.

The administering in the methods herein is not limited to any particular route of administration. For example, in some embodiments, the administering can be orally, nasally, transdermally, pulmonary, inhalationally, buccally, sublingually, intraperitoneally, subcutaneously, intramuscularly, intravenously, rectally, intrapleurally, intrathecally and parenterally.

The compounds of the present disclosure can be used as a monotherapy or in a combination therapy. In some embodiments, the combination therapy includes treating the subject with a targeted therapeutic agent, chemotherapeutic agent, therapeutic antibody, radiation, cell therapy, and/or immunotherapy. In some embodiments, the combination therapy includes administering to the subject an immunotherapy, such as an anti-PD-1, anti-PDL-1 antibody, anti-CTLA-4 and/or anti-4-1BB antibody.

It is to be understood that both the foregoing summary and the following detailed description are exemplary and explanatory only, and are not restrictive of the invention herein.

### DETAILED DESCRIPTION

As detailed herein, the present disclosure is based in part on the discovery that certain novel compounds can be PARP7 inhibitors, which are useful for treating various diseases or disorders such as cancer. In various embodiments, the present disclosure provides novel compounds, compositions, methods of preparing, and methods of using related to the discovery.

### Compounds

Some embodiments of the present disclosure are directed to novel compounds. The compounds herein typically are PARP inhibitors, in particular, PARP7 inhibitors, and are useful for treating various diseases or disorders, such as those described herein, e.g., cancer.

In some embodiments, the present disclosure provides a compound of Formula I, or a pharmaceutically acceptable salt thereof:

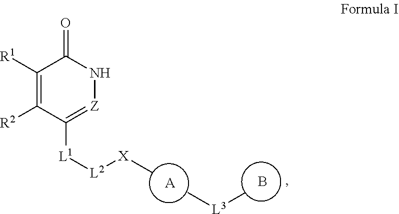

wherein:
Z is N or C, preferably N,
R¹ is hydrogen, halogen, CN, OR¹⁰, SR¹¹, S(O) R¹², S(O)₂R¹³, optionally substituted alkyl, optionally substituted alkenyl, optionally substituted alkynyl, optionally substituted carbocyclyl, optionally substituted heteroaryl, or optionally substituted heterocyclyl;
R² is hydrogen, halogen, CN, OR¹⁰, SR¹¹, S(O) R¹², S(O)₂R¹³, NR¹⁴R¹⁵, optionally substituted alkyl, optionally substituted alkenyl, optionally substituted alkynyl, optionally substituted carbocyclyl, optionally substituted phenyl, optionally substituted heteroaryl, or optionally substituted heterocyclyl;
L¹ and L² are independently null, O, S, S(O), S(O)₂, NR¹⁶, C(O), C(O) O, C(O) NR¹⁶, OC(O) NR¹⁶, S(O)₂NR¹⁶, NR¹⁷C(O) NR¹⁶, NR¹⁷S(O)₂NR¹⁶, optionally substituted alkylene, optionally substituted alkenylene, optionally substituted alkynylene, optionally substituted heteroalkylene, optionally substituted carbocyclylene, optionally substituted heterocyclylene, optionally substituted phenylene, or optionally substituted heteroarylene, preferably, L¹ and L² are not both null,
X is null, C(O), G¹-C(O)-G², S(O), S(O)₂, or G¹-S(O)₂-G², wherein G¹ and G² are each independently null, O, NH, optionally substituted C₁₋₄ alkylene, or optionally substituted C₁₋₄ heteroalkylene, or G¹ and G², together with the intervening atoms, are joined to form an optionally substituted 4-7 membered ring structure,
Ring A is an optionally substituted carbocyclic or heterocyclic ring,
L³ is null, O, S, S(O), S(O)₂, NR¹⁶, optionally substituted C₁₋₄ alkylene, or optionally substituted C₁₋₄ heteroalkylene,
Ring B is an optionally substituted aryl or heteroaryl ring, or R¹ and R², together with the intervening atoms, are joined to form an optionally substituted cyclic structure;
or R² and L¹, together with the intervening atoms, are joined to form an optionally substituted cyclic structure;
or L¹ and L², together with the intervening atoms, are joined to form an optionally substituted cyclic structure;
or R¹, R², and L¹, together with the intervening atoms, are joined to form an optionally substituted cyclic structure;
or when L³ is null, ring A and ring B together represent an optionally substituted cyclic structure having one ring or at least two rings, e.g., a bicyclic structure;
wherein:
each of R¹⁰, R¹¹, R¹², and R¹³ at each occurrence is independently selected from hydrogen, optionally substituted alkyl, optionally substituted carbocyclyl, or optionally substituted heterocyclyl; and
each of R¹⁴, R¹⁵, R¹⁶, and R¹⁷ at each occurrence is independently selected from hydrogen, nitrogen protecting group, optionally substituted alkyl, optionally substituted cycloalkyl, or optionally substituted heterocyclyl.

In some embodiments, the compound of Formula I (including any of the applicable sub-formulae as described herein) can have stereoisomers. In such embodiments, the compound of Formula I can exist in the form of an individual enantiomer, diastereomer, and/or geometric isomer, as applicable, or a mixture of stereoisomers, including racemic mixtures and mixtures enriched in one or more stereoisomers. For example, in some embodiments, when applicable, the compound of Formula I (including any of the applicable sub-formulae as described herein) can exist as an isolated individual enantiomer substantially free (e.g., with less than 20%, less than 10%, less than 5%, less than 1%, by weight, by HPLC area, or both, or with a non-detectable amount) of the other enantiomer. In some embodiments, when applicable, the compound of Formula I (including any of the applicable sub-formulae as described herein) can exist as an individual enantiomer having an enantiomeric excess (“ee”) of greater than 60%, e.g., greater than 80% ee, greater than 85% ee, greater than 90% ee, greater than 95% ee, greater than 98% ee, greater than 99% ee, or the other enantiomer is non-detectable.

Typically, in Formula I, Z is N. Thus, the compound of Formula I can be typically characterized as having a Formula I-1:

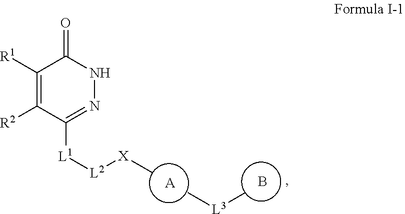

wherein the variables R¹, R², L¹, L², L³, X, ring A, and ring B include any of those described herein in any combinations.

In some embodiments, in Formula I, Z can also be C.

In some embodiments, R¹ in Formula I (e.g., any of the applicable sub-formulae as described herein, such as I-1, I-B, I-B-1, I-B-2, I-B-3, I-B-1-E1, I-B-1-E2, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) is hydrogen. Typically, when R¹ is hydrogen, R² is not hydrogen.

In some embodiments, R¹ in Formula I (e.g., any of the applicable sub-formulae as described herein) is halogen, such as F, Cl, or Br.

In some embodiments, R¹ in Formula I (e.g., any of the applicable subformulae herein) is CN.

In some embodiments, R¹ in Formula I (e.g., any of the applicable subformulae herein) is an optionally substituted C₁₋₆ alkyl. In some embodiments, R¹ in Formula I (e.g., any of the applicable subformulae herein) is an optionally substituted C₂₋₆ alkenyl. In some embodiments, R¹ in Formula I (e.g., any of the applicable subformulae herein) is an optionally substituted C₂₋₆ alkynyl. In some embodiments, R¹ in Formula I (e.g., any of the applicable subformulae herein) is an optionally substituted C₃₋₈ carbocyclyl. For example, in some preferred embodiments, R¹ in Formula I (e.g., any of the applicable subformulae herein) is an optionally substituted C₁₋₄ alkyl, C₂₋₄ alkenyl, C₂₋₄ alkynyl, or C₃₋₆ cycloalkyl. In some embodiments, R¹ is C₁₋₄ alkyl, C₂₋₄ alkenyl, C₂₋₄ alkynyl, or C₃₋₆ cycloalkyl, each of which is optionally substituted with one or more (e.g., 1-3) substituents independently selected from F, OH, oxo, C₁₋₄ alkyl optionally substituted with 1-3 F, or C₁₋₄ alkoxy optionally substituted with 1-3 F. For example, in some embodiments, R¹ is C₁₋₄ alkyl optionally substituted with 1-3 F, such as methyl, ethyl, isopropyl, CHF₂, CF₃, etc. In some embodiments, R¹ is C₂₋₄ alkynyl, such as C₂ alkynyl. In some embodiments, R¹ is C₃₋₆ cycloalkyl, such as cyclopropyl.

In some embodiments, R¹ in Formula I (e.g., any of the applicable subformulae herein) is OR¹⁰, wherein R¹⁰ is defined herein. For example, in some embodiments, R¹ in Formula I is OR¹⁰, wherein R¹⁰ is hydrogen, C₁₋₄ alkyl, or C₃₋₆ cycloalkyl, wherein the C₁₋₄ alkyl or C₃₋₆ cycloalkyl is optionally substituted with one or more (e.g., 1-3) substituents independently selected from F, OH, oxo, C₁₋₄ alkyl optionally substituted with 1-3 F, and C₁₋₄ alkoxy optionally substituted with 1-3 F. For example, in some embodiments, R¹ can be OCH₃. In some embodiments, R¹ can be a C₁₋₄ alkoxy optionally substituted with 1-3 F, such as OCH₂CF₂H.

In some embodiments, R¹ in Formula I (e.g., any of the applicable subformulae herein) is SR¹¹, wherein R¹¹ is defined herein. For example, in some embodiments, R¹ in Formula I is SR¹¹, wherein R¹¹ is hydrogen, C₁₋₄ alkyl, or C₃₋₆ cycloalkyl, wherein the C₁₋₄ alkyl or C₃₋₆ cycloalkyl is optionally substituted with one or more (e.g., 1-3) substituents independently selected from F, OH, oxo, C₁₋₄ alkyl optionally substituted with 1-3 F, and C₁₋₄ alkoxy optionally substituted with 1-3 F. For example, in some embodiments, R¹ can be SCH₃.

In some embodiments, R¹ in Formula I (e.g., any of the applicable subformulae herein) can be S(O) R¹² or S(O)₂R¹³, wherein R¹² and R¹³ are defined herein. For example, in some embodiments, R¹² or R¹³ can be hydrogen, C₁₋₄ alkyl, or C₃₋₆ cycloalkyl, wherein the C₁₋₄ alkyl or C₃₋₆ cycloalkyl is optionally substituted with one or more (e.g., 1-3) substituents independently selected from F, OH, oxo, C₁₋₄ alkyl optionally substituted with 1-3 F, and C₁₋₄ alkoxy optionally substituted with 1-3 F.

In any of the embodiments described herein, unless specified or otherwise contrary from context, R¹ in Formula I (e.g., any of the applicable subformulae herein, such as I-1, I-B, I-B-1, I-B-2, I-B-3, I-B-1-E1, I-B-1-E2, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) can be hydrogen, CH₃, ethyl, isopropyl, cyclopropyl, CN, OCH₃, SCH₃, CF₃, F, Cl, Br, CF₂H,

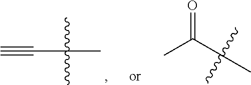

or OCH₂CF₂H.

In some specific embodiments, the compound of Formula I can be characterized as having a Formula I-2:

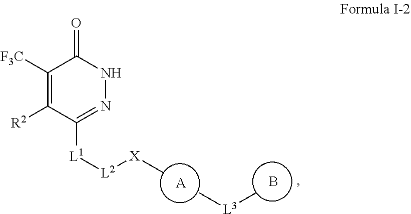

wherein the variables R², L¹, L², L³, X, ring A, and ring B include any of those described herein in any combinations.

In some embodiments, R² in Formula I (e.g., any of the applicable subformulae herein, such as I-1, 1-2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) can be hydrogen.

In some embodiments, R² in Formula I (e.g., any of the applicable subformulae herein) can be an optionally substituted C₁₋₆ alkyl. In some embodiments, R² in Formula I (e.g., any of the applicable subformulae herein) is an optionally substituted C₂₋₆ alkenyl. In some embodiments, R² in Formula I (e.g., any of the applicable subformulae herein) is an optionally substituted C₂₋₆ alkynyl. In some embodiments, R² in Formula I (e.g., any of the applicable subformulae herein) is an optionally substituted C₃₋₈ carbocyclyl.

In some embodiments, R² in Formula I (e.g., any of the applicable subformulae herein) is C₁₋₄ alkyl, C₂₋₄ alkenyl, C₂₋₄ alkynyl, or C₃₋₆ cycloalkyl, each of which is optionally substituted with one or more (e.g., 1-5 or 1-3) substituents independently selected from (1) halo (preferably, F) or CN, (2) OH, (3) NG³G⁴, (4) oxo, (5) G⁵, and (6) OG⁵, wherein: G³ and G⁴ are independently hydrogen or G⁵, wherein G⁵ is defined herein. In some embodiments, R² in Formula I (e.g., any of the applicable subformulae herein) is C₁₋₄ alkyl optionally substituted with one or more (e.g., 1-5 or 1-3) substituents independently selected from (1) F, (2) OH, and (6) OG⁵, wherein G⁵ is defined herein. In some embodiments, R² in Formula I (e.g., any of the applicable subformulae herein) is C₂₋₄ alkenyl or C₂₋₄ alkynyl, each optionally substituted with one or more (e.g., 1-5 or 1-3) substituents independently selected from (1) F and (5) G⁵, wherein G⁵ is defined herein. In some embodiments, R² in Formula I (e.g., any of the applicable subformulae herein) is C₃₋₆ cycloalkyl, optionally substituted with one or more (e.g., 1-5 or 1-3) substituents independently selected from (1) F, (2) OH, (5) G⁵, and (6) OG⁵, wherein G⁵ is defined herein. In some embodiments, G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G⁴, (iii)4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B},
wherein
G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C}; and
G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F. It should be noted that in the expression “N(C₁₋₄ alkyl) (C₁₋₄ alkyl)” as used herein, the two C₁₋₄ alkyl can be the same or different.

In some embodiments, G⁵ at each occurrence is independently (i) C₁₋₄ alkyl optionally substituted with 1-5 (e.g., 1, 2, or 3) G^{A1}, (ii) C₃₋₆ cycloalkyl optionally substituted with 1-5 (e.g., 1, 2, or 3) G^{A1}, (iii)4-6 membered monocyclic heterocyclyl having 1-2 ring heteroatoms, each independently selected from N, O, and S, which is optionally substituted with 1-5 (e.g., 1, 2, or 3) G^{A1}, (iv) phenyl optionally substituted with 1-5 (e.g., 1, 2, or 3) G^{B1}, or (v)5 or 6 membered heteroaryl having 1-3 ring heteroatoms, which is optionally substituted with 1-5 (e.g., 1, 2, or 3) G^{B1},
wherein
G^{A1} at each occurrence is independently F; oxo; C₁₋₄ alkyl optionally substituted with 1-3 F; OH; NH₂; NH (C₁₋₄ alkyl); N(C₁₋₄ alkyl) (C₁₋₄ alkyl); or C₁₋₄ alkoxy optionally substituted with 1-3 F; and
G^{B1} at each occurrence is independently halo (preferably, F, Cl, or Br); CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F;
wherein GCI at each occurrence is independently F; OH; C₁₋₃ alkyl (preferably methyl) optionally substituted with 1-3 F; or C₁₋₃ alkoxy (preferably methoxy) optionally substituted with 1-3 F.

In some embodiments, G⁵ at each occurrence is independently (i) C₁₋₄ alkyl optionally substituted with 1-5 (e.g., 1, 2, or 3) G^{A2}, or (ii) C₃₋₆ cycloalkyl optionally substituted with 1-5 (e.g., 1, 2, or 3) G^{A2},
wherein
G^{A2} at each occurrence is independently F; oxo; C₁₋₃ alkyl (preferably methyl) optionally substituted with 1-3 F; OH; NH₂; NH (C₁₋₃ alkyl), preferably, NHCH₃; N(C₁₋₃ alkyl) (C₁₋₃ alkyl), preferably, N(CH₃)₂; or C₁₋₃ alkoxy (preferably methoxy) optionally substituted with 1-3 F.

For example, in some embodiments, R² in Formula I (e.g., any of the applicable subformulae herein) is an optionally substituted C₁₋₄ alkyl, such as methyl, methoxymethyl, CF₃, etc. In some embodiments, R² in Formula I (e.g., any of the applicable subformulae herein) is an optionally substituted C₂₋₄ alkenyl. In some embodiments, R² in Formula I (e.g., any of the applicable subformulae herein) is an optionally substituted C₂₋₄ alkynyl, such as

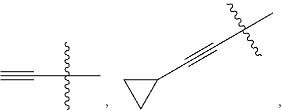

etc. In some embodiments, R² in Formula I (e.g., any of the applicable subformulae herein) is an optionally substituted C₃₋₆ cycloalkyl, such as cyclopropyl.

In some embodiments, R² in Formula I (e.g., any of the applicable subformulae herein) is NR¹⁴R¹⁵, wherein R¹⁴ and R¹⁵ are defined herein. For example, in some embodiments, R¹⁴ and R¹⁵ are independently selected from (i) hydrogen, (ii) C₁₋₄ alkyl optionally substituted with 1-5 (e.g., 1, 2, or 3) G⁴, (iii) C₃₋₆ cycloalkyl optionally substituted with 1-5 (e.g., 1, 2, or 3) G^{A}, and (iv)4-8 membered heterocyclyl having 1-3 ring heteroatoms, each independently selected from N, O, and S, which is optionally substituted with 1-5 (e.g., 1, 2, or 3) G^{A},
wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C};
wherein G^{C} at each occurrence is independently F, OH, C₁₋₄ alkyl optionally substituted with 1-3 F, or C₁₋₄ alkoxy optionally substituted with 1-3 F.

In some embodiments, R¹⁴ and R¹⁵ are independently selected from (i) hydrogen, (ii) C₁₋₄ alkyl optionally substituted with 1-5 (e.g., 1, 2, or 3) G^{A1}, (iii) C₃₋₆ cycloalkyl optionally substituted with 1-5 (e.g., 1, 2, or 3) G^{A1}, and (iv)4-6 membered monocyclic heterocyclyl having 1-3 ring heteroatoms, each independently selected from N, O, and S, which is optionally substituted with 1-5 (e.g., 1, 2, or 3) G^{A1},
wherein G^{A1} at each occurrence is independently F; oxo; C₁₋₄ alkyl optionally substituted with 1-3 F; OH; NH₂; NH (C₁₋₄ alkyl); N(C₁₋₄ alkyl) (C₁₋₄ alkyl); or C₁₋₄ alkoxy optionally substituted with 1-3 F. For example, in some embodiments, R² in Formula I is NH₂; NH (C₁₋₄ alkyl); or N(C₁₋₄ alkyl) (C₁₋₄ alkyl). In some specific embodiments, R² in Formula I is NH₂ or NHCH₃.

In some embodiments, R² in Formula I (e.g., any of the applicable subformulae herein) can also be an optionally substituted heterocyclyl.

For example, in some embodiments, R² in Formula I (e.g., any of the applicable subformulae herein) is an optionally substituted 4-10 membered heterocyclyl having 1-3 ring heteroatoms, each independently selected from N, O, and S, which can be saturated, partially unsaturated, and can include a fused, spiro, or bridged ring system. In some embodiments, the 4-10 membered heterocyclyl is a 4-6 membered monocyclic heterocyclic ring. In some embodiments, the 4-10 membered heterocyclyl is a 6-10 membered fused, spiro, or bridged bicyclic heterocyclic ring. A fused bicyclic heterocyclic ring can include one ring that is aryl or heteroaryl, so long as the bicyclic heterocyclic ring as a whole is not fully aromatic. Typically, the 4-10 membered heterocyclyl ring includes 1-3 ring heteroatoms, such as one or two ring heteroatoms, each independently O, N, or S. When substituted, the 4-10 membered heterocyclyl can be typically substituted with 1-5 (e.g., 1, 2, or 3) G^{A}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{c};
wherein G^{C} at each occurrence is independently F, OH, C₁₋₄ alkyl optionally substituted with 1-3 F, or C₁₋₄ alkoxy optionally substituted with 1-3 F. In some embodiments, the 4-10 membered heterocyclyl can be a 4-8 membered mono or bicyclic (fused, spiro, or bridged bicyclic) heterocyclyl having 1-3 ring heteroatoms, such as one or two ring heteroatoms, each independently selected from N, O, and S. For example, in some embodiments, R² in Formula I (e.g., any of the applicable subformulae herein) can be a spiro-bicyclic heterocyclyl, such as a 6-membered spirobicyclic ring, such as

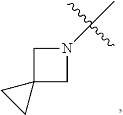 [SMILES: *N1CC2(CC2)C1]

6-membered fused bicyclic ring, such as

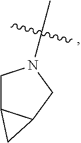 [SMILES: *N1CC2CC2C1]

or 7-membered spirobicyclic ring, such as

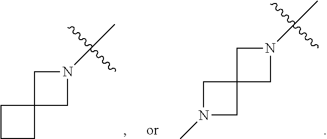

In some embodiments, R² in Formula I (e.g., any of the applicable subformulae herein) can be a monocyclic 4-7 membered heterocyclic ring, such as

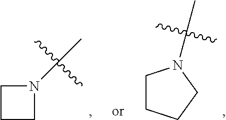 [SMILES: *N1CCC1 ; *N1CCCC1]

which is optionally substituted with one or more (e.g., 1 or 2) substituents described herein, for example, the substituents can each be independently selected from halo (preferably, F); CN; OH; NH₂; NH (C₁₋₄ alkyl); N(C₁₋₄ alkyl) (C₁₋₄ alkyl); and C₁₋₄ alkyl optionally substituted with 1-3 F. In some embodiments, the subsitutents can each be independently selected from F, CN, NH (CH₃), N(CH₃)₂, CHF₂, and methyl.

In some embodiments, R² in Formula I (e.g., any of the applicable subformulae herein) can be OR¹⁰, wherein R¹⁰ is defined herein. For example, in some embodiments, R¹⁰ is (i) C₁₋₄ alkyl optionally substituted with 1-5 (e.g., 1, 2, or 3) G⁴³, (ii) C₃₋₆ cycloalkyl optionally substituted with 1-5 (e.g., 1, 2, or 3) G⁴³, or (iii)4-8 membered heterocyclyl having 1-3 ring heteroatoms, each independently selected from N, O, and S, which is optionally substituted with 1-5 (e.g., 1, 2, or 3) G⁴³, wherein G⁴³ is defined herein. In some embodiments, R² in Formula I (e.g., any of the applicable subformulae herein) can be NHR¹⁵, wherein R¹⁵ is defined herein. For example, in some embodiments, R¹⁵ is (i) C₁₋₄ alkyl optionally substituted with 1-5 (e.g., 1, 2, or 3) G⁴³, (ii) C₃₋₆ cycloalkyl optionally substituted with 1-5 (e.g., 1, 2, or 3) G^{A3}, or (iii)4-8 membered heterocyclyl having 1-3 ring heteroatoms, each independently selected from N, O, and S, which is optionally substituted with 1-5 (e.g., 1, 2, or 3) G^{A3}, wherein G⁴³ is defined herein. In the foregoing definition of R¹⁰ and R¹⁵, G^{A3} at each occurrence can be independently halo (preferably, F) or CN; oxo; C₁₋₄ alkyl optionally substituted with 1-5 (e.g., 1, 2, or 3) G^{C3}; C₂₋₄ alkenyl optionally substituted with 1-5 (e.g., 1, 2, or 3) G^{C3}; C₂₋₄ alkynyl optionally substituted with 1-5 (e.g., 1, 2, or 3) G^{C3}; OH; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C3}; or a 3-8 membered ring optionally substituted with 1-5 (e.g., 1, 2, or 3) G^{C3};

wherein G^{C3} at each occurrence is independently is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F. As used herein, unless otherwise specified or contrary from context, a “3-8 membered ring” should be understood as encompassing monocyclic and bicyclic (fused, spiro, or bridged) ring having 3-8 ring atoms, which can be saturated, partially unsaturated, or aromatic ring, which optionally includes one or more ring heteroatoms independently N, O, or S, wherein the ring carbon, N, or S atom may be optionally oxidized, such as in the form of C(═O), N-oxide, SO, or SO₂. Other membered ring should be understood similarly. Examples of 3-8 membered ring include without limitation cyclopropyl, cyclobutyl, azetidinyl, oxetanyl, tetrahydrofuranyl, tetrahydropyranyl, phenyl, 5 or 6 membered heteroaryl such as pyrazole, etc. Non-limiting examples of C₁₋₄ heteroalkyl having 1 or 2 heteroatoms independently O, N, or S include C₁₋₄ alkoxy, NH (C₁₋₄ alkyl), N(C₁₋₃ alkyl) (C₁₋₃ alkyl), wherein each C₁₋₃ alkyl is independently selected, provided that the total number of carbons is no greater than 4, hydroxyl or NH₂ substituted C₁₋₄ alkyl, methoxy substituted C₁-3 alkyl, NMe₂ substituted C₁-2 alkyl, etc.

In some embodiments, in Formula I (e.g., any of the applicable subformulae herein), R² is OR¹⁰ as defined herein, such as

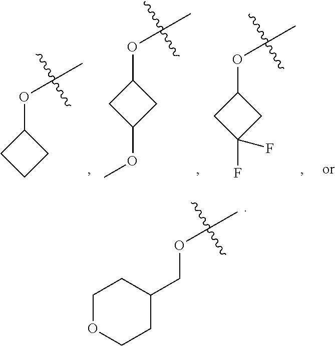

In some embodiments, in Formula I (e.g., any of the applicable subformulae herein), R² is NHR¹⁵ as defined herein, such as

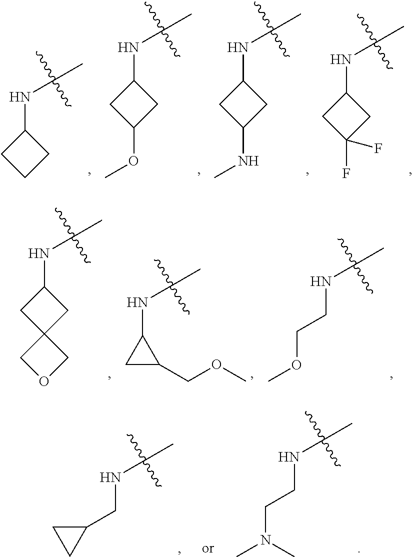

In some embodiments, R² in Formula I (e.g., any of the applicable subformulae herein) can be 4-10 membered heterocyclyl having 1-3 ring heteroatoms, each independently selected from N, O, and S, which is optionally substituted with 1-5 (e.g., 1, 2, or 3) G^{A3}, wherein G^{A3} at each occurrence is independently halo (preferably, F) or CN; oxo; C₁₋₄ alkyl optionally substituted with 1-5 (e.g., 1, 2, or 3) G^{C3}; C₂₋₄ alkenyl optionally substituted with 1-5 (e.g., 1, 2, or 3) G^{C3}; C₂₋₄ alkynyl optionally substituted with 1-5 (e.g., 1, 2, or 3) G^{C3}; OH; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C3}; or a 3-8 membered ring optionally substituted with 1-5 (e.g., 1, 2, or 3) G^{C3};

wherein G^{C3} at each occurrence is independently is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F. For example, in some embodiments, R² in Formula I (e.g., any of the applicable subformulae herein) can be 4-8 membered mono or bicyclic (fused, spiro, or bridged bicyclic) heterocyclyl having one or two ring heteroatoms, each independently selected from N, O, and S, such as

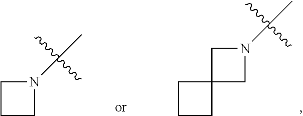 [SMILES: *N1CCC1 ; *N1CC2(CCC2)C1]

which is optionally substituted with 1-2 G^{A3}. For example, in some embodiments, R² in Formula I (e.g., any of the applicable subformulae herein) can be

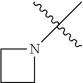 [SMILES: *N1CCC1]

or a substituted azetidine selected from

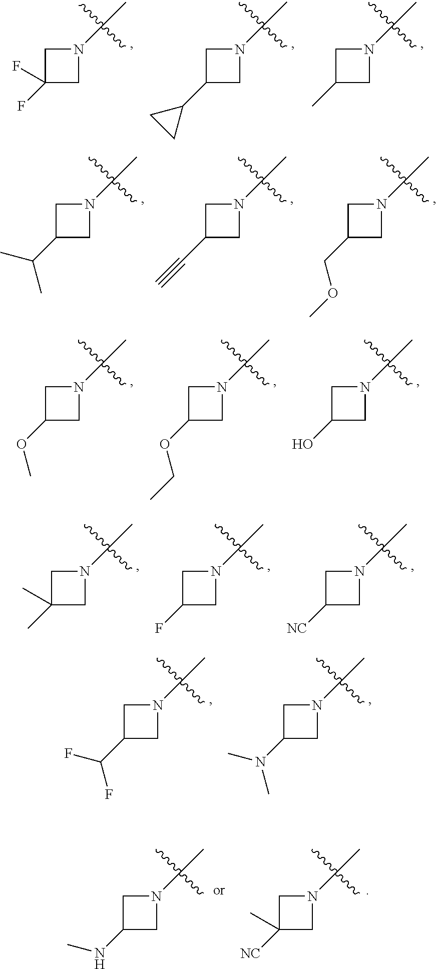

In some embodiments, R² in Formula I (e.g., any of the applicable subformulae herein) can be an spirobicyclic ring having an azetidine ring, which is optionally substituted, such as

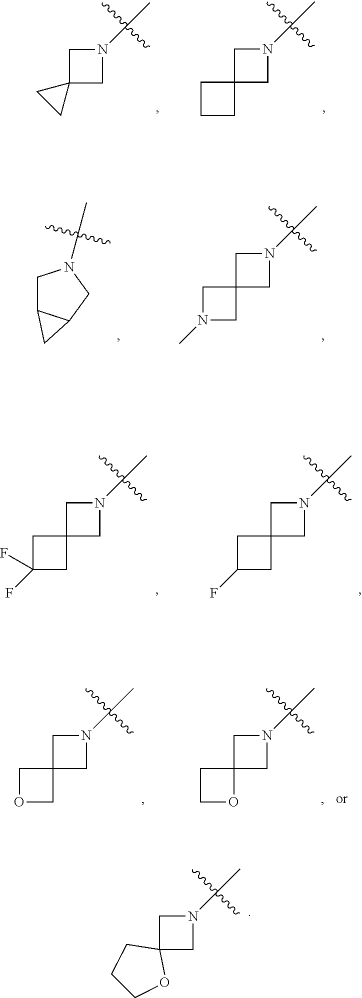

In any of the embodiments described herein, unless specified or otherwise contrary from context, R² in Formula I (e.g., any of the applicable subformulae herein, such as I-1, I-2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) can be hydrogen, CH₃, CF₃,

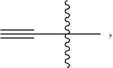 [SMILES: *C#C]

NH₂, NHCH₃,

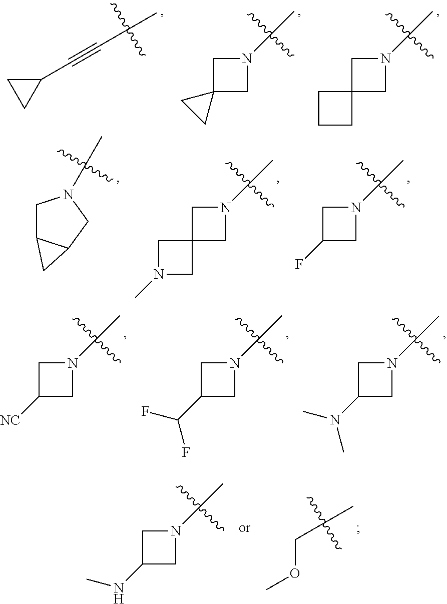

### or R² is

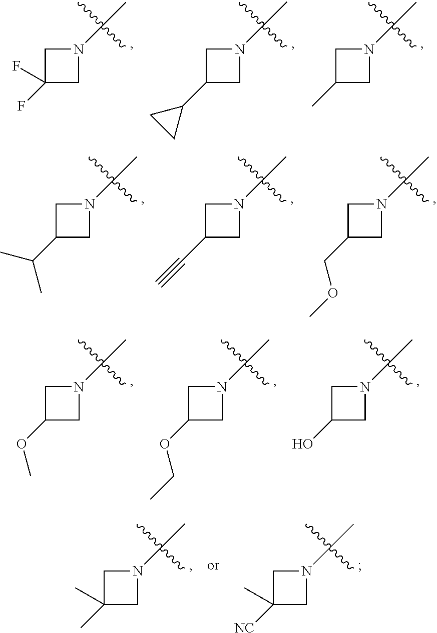

### or R² is

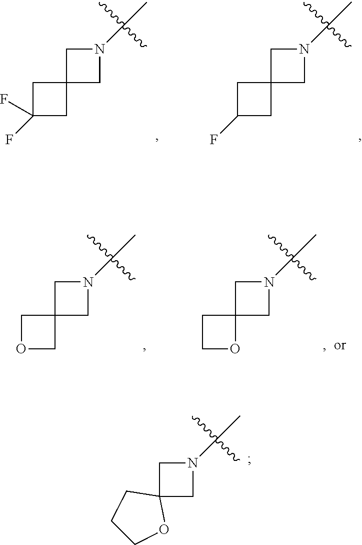 [SMILES: *N1CC2(C1)CC(F)(F)C2 ; *N1CC2(CC(F)C2)C1 ; *N1CC2(COC2)C1 ; *N1CC2(CCO2)C1 ; *N1CC2(CCCO2)C1]

### or R² is

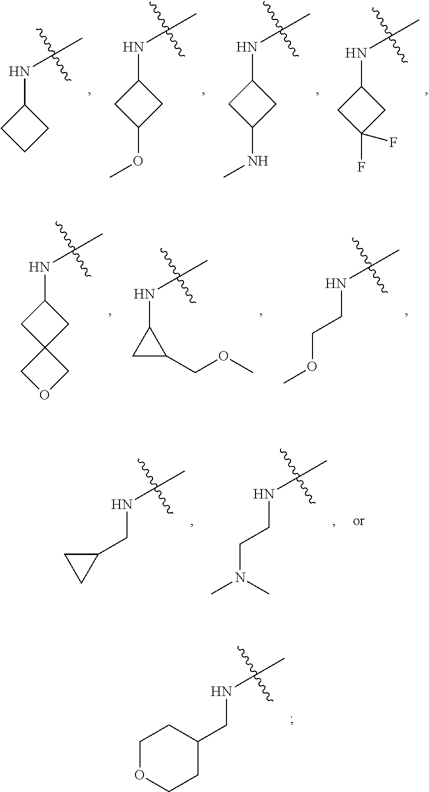

### or R² is

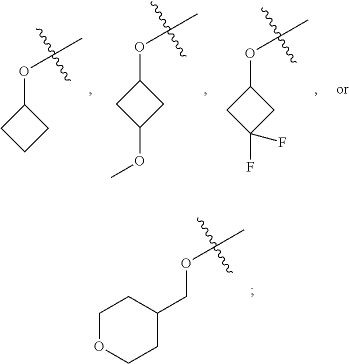

or R² is cyclopropyl.

In some embodiments, in Formula I (e.g., any of the applicable subformulae herein), R¹ and R², together with the intervening atoms, are joined to form an optionally substituted cyclic structure. For example, in some embodiments, compounds of Formula I can be characterized as having a Formula I-A:

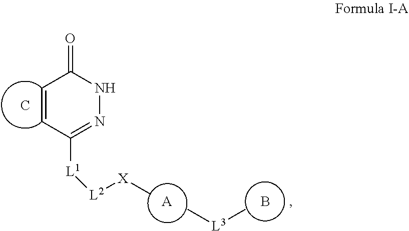

wherein Ring C represents an optionally substituted ring structure, such as an optionally substituted phenyl ring, optionally substituted heteroaryl, optionally substituted carbocyclyl or heterocyclyl ring, and wherein the variables L¹, L², L³, X, ring A, and ring B include any of those described herein in any combinations. Typically, ring C is an optionally substituted phenyl or heteroaryl ring.

In some embodiments, in Formula I (e.g., any of the applicable subformulae herein), L¹ and R², together with the intervening atoms, are joined to form an optionally substituted cyclic structure. For example, in some embodiments, compounds of Formula I can be characterized as having a Formula I-B:

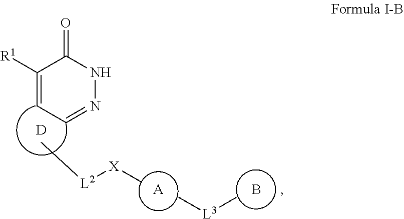

wherein Ring D represents an optionally substituted ring structure, such as an optionally substituted phenyl ring, optionally substituted heteroaryl, optionally substituted carbocyclyl or heterocyclyl ring, and wherein the variables R¹, L², L³, X, ring A, and ring B include any of those described herein in any combinations. Typically, ring D is an optionally substituted carbocyclic or heterocyclic ring.

In some embodiments, in Formula I (e.g., any of the applicable subformulae herein), R¹, R², and L¹, together with the intervening atoms, are joined to form an optionally substituted cyclic structure:

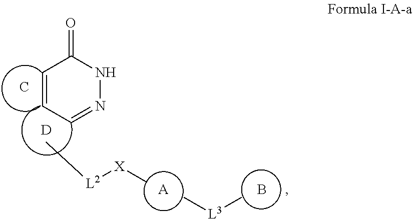

wherein Ring C and D each independently represents an optionally substituted ring structure, such as an optionally substituted phenyl ring, optionally substituted heteroaryl, optionally substituted carbocyclyl or heterocyclyl ring, and wherein the variables L², L³, X, ring A, and ring B include any of those described herein in any combinations. Typically, ring C and ring D are not aromatic rings at the same time. For example, typically, ring C is an optionally substituted phenyl or heteroaryl ring and ring D is an optionally substituted carbocyclic or heterocyclic ring.

In some embodiments, R¹ and R², R² and L¹, or R¹, R², and L¹ do not form a ring among each other.

In some embodiments, in Formula I (e.g., any of the applicable subformulae herein, such as I-1, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), R¹ and R², together with the intervening atoms, are joined to form an optionally substituted phenyl ring or an optionally substituted 5 or 6 membered heteroaryl ring having 1-3 ring heteroatoms, each independently selected from N, O, and S. Thus, in such embodiments, ring C in Formula I-A or as applicable in Formula I-A-a, can be an optionally substituted phenyl ring or an optionally substituted 5 or 6 membered heteroaryl ring having 1-3 ring heteroatoms, each independently selected from N, O, and S.

In some embodiments, in Formula I (e.g., any of the applicable subformulae herein), R¹ and R², together with the intervening atoms, are joined to form a phenyl ring,

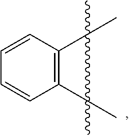

which is optionally substituted. In some embodiments, R¹ and R², together with the intervening atoms, are joined to form a pyridine ring, e.g.,

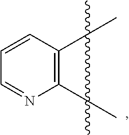

which is optionally substituted. Unless otherwise specified herein, either of the two connecting points of this pyridyl fragment can be connected to the carbonyl group in Formula I. For example, in some embodiments, the top connecting point of this pyridyl fragment is to the carbonyl group in Formula I, see e.g., Formula I-A-2. In some embodiments, the bottom connecting point of this pyridyl fragment is to the carbonyl group in Formula I, see e.g., Compound 126. Terms such as top, bottom, etc. should be understood as the relative position as drawn. In some embodiments, R¹ and R², together with the intervening atoms, are joined to form a pyrrole ring, e.g.,

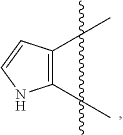

which is optionally substituted. Similarly, unless otherwise specified herein, either of the two connecting points of this pyrrolyl fragment can be connected to the carbonyl group in Formula I. When substituted, the phenyl ring or 5 or 6 membered heteroaryl ring, such as the pyridine or pyrrole ring, can be typically substituted with 1-5 (e.g., 1, 2, or 3) G^{B}, wherein G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};

wherein G^{C} at each occurrence is independently F; OH; C₁₋₄ alkyl optionally substituted with 1-3 F; or C₁₋₄ alkoxy optionally substituted with 1-3 F.

In some embodiments, when substituted, the phenyl ring or 5 or 6 membered heteroaryl ring, such as the pyridine or pyrrole ring, can be substituted with 1-5 (e.g., 1, 2, or 3) G^{B1}, wherein G^{B1} at each occurrence is independently F; Cl; Br; CN; C₁₋₄ alkyl optionally substituted with 1-3 F; OH; C₃₋₆ cycloalkyl optionally substituted with 1-3 G¹; 4-6 membered monocyclic heterocyclyl having 1-2 ring heteroatoms, each independently selected from N, O, and S, which is optionally substituted with 1-3 G^{C1}; or C₁₋₄ alkoxy optionally substituted with 1-3 F; wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F.

In some embodiments, when substituted, the phenyl ring or 5 or 6 membered heteroaryl ring, such as the pyridine or pyrrole ring, can be substituted with 1-5 (e.g., 1, 2, or 3) G^{B3}, wherein G^{B3} at each occurrence is independently F, Cl, Br, CN, C₁₋₄ alkyl optionally substituted with 1-5 (e.g., 1, 2, or 3) G^{C3}, C₂₋₄ alkenyl optionally substituted with 1-5 (e.g., 1, 2, or 3) G^{C3}, C₂₋₄ alkynyl optionally substituted with 1-5 (e.g., 1, 2, or 3) G^{C3}, OH, C₃₋₆ cycloalkyl optionally substituted with 1-5 (e.g., 1, 2, or 3) G^{C3}, 4-6 membered heterocyclyl having 1-3 ring heteroatoms, each independently selected from N, O, and S, which is optionally substituted with 1-5 (e.g., 1, 2, or 3) G^{C}, NH₂, NH (C₁₋₄ alkyl), N(C₁₋₄ alkyl) (C₁₋₄ alkyl), C₁₋₄ alkoxy optionally substituted with 1-5 (e.g., 1, 2, or 3) G^{C3}, C₃₋₆ cycloalkoxy optionally substituted with 1-5 (e.g., 1, 2, or 3) G^{C3}, 4-6 membered heterocycloalkoxy optionally substituted with 1-5 (e.g., 1, 2, or 3) G^{C3}, or a 5 or 6-membered heteroaryl optionally substituted with 1-5 (e.g., 1, 2, or 3) G^{C3};

wherein G^{C3} at each occurrence is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F.

In some embodiments, in Formula I (e.g., any of the applicable subformulae herein), R¹ and R², together with the intervening atoms, are joined to form a phenyl ring,

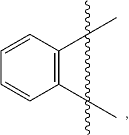

which is optionally substituted with one or more (e.g., 1-5 or 1-3) substituents independently selected from F, Cl, C₁₋₄ alkyl optionally substituted with 1-3 F, cyclopropyl and cyclobutyl. In some embodiments, in Formula I (e.g., any of the applicable subformulae herein), R¹ and R², together with the intervening atoms, are joined to form a phenyl ring,

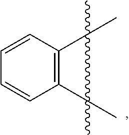

which is optionally substituted with one or more (e.g., 1-5 or 1-3, more preferably 1 or 2) substituents independently selected from F, Cl, C₁₋₄ alkyl optionally substituted with 1-3 F, cyclopropyl cyclobutyl,

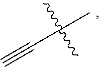 [SMILES: *C#C]

### CN,

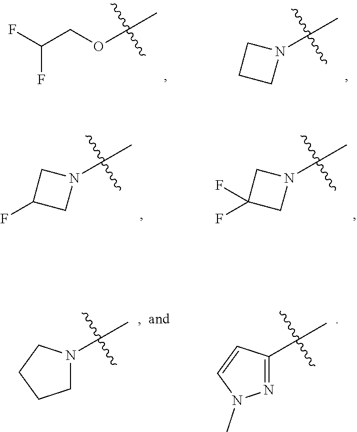

In some specific embodiments, in Formula I (e.g., any of the applicable subformulae herein, such as I-1, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), R¹ and R², together with the intervening atoms, are joined to form a ring structure selected from:

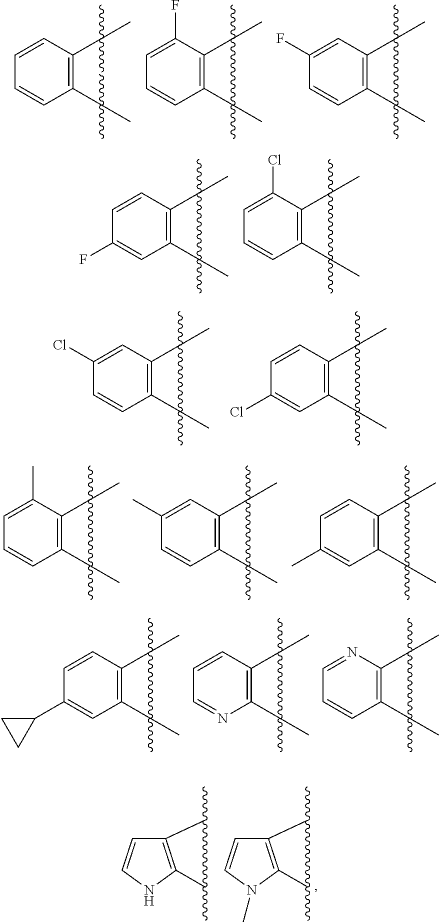

or selected from:

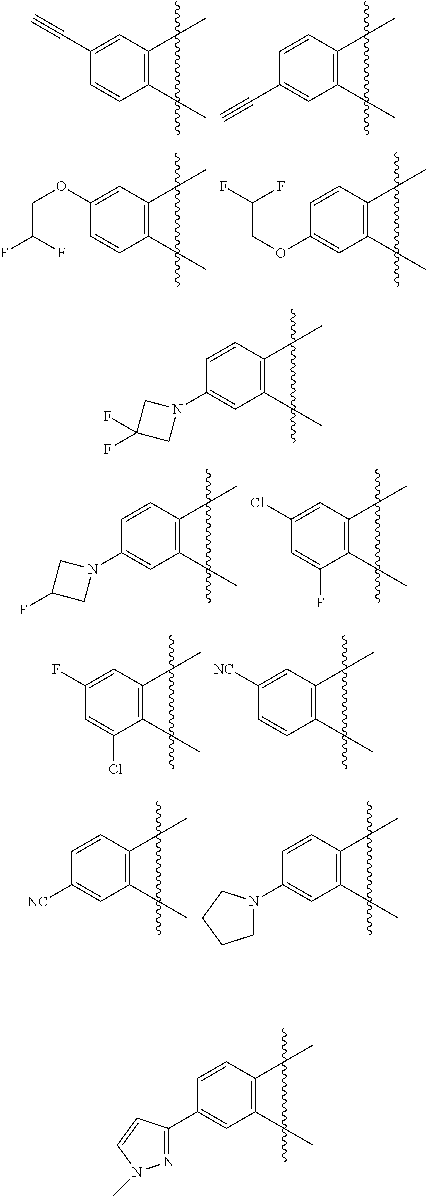

wherein the top connecting point of the fragments above is to the carbonyl group in Formula I.

In some embodiments, the compounds of Formula I can be characterized as having the following Formula I-A-1, I-A-2, or I-A-3:

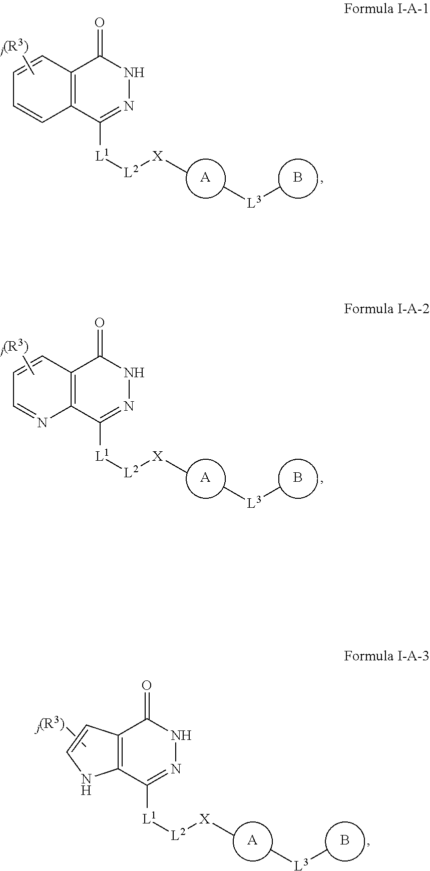

wherein:
the variables L¹, L², L³, X, ring A, and ring B include any of those described herein in any combinations; and wherein
j is 0, 1, 2, or 3, and
R³ at each occurrence is independently halo (preferably, F, Cl, or Br), CN, C₁₋₄ alkyl optionally substituted with 1-5 (e.g., 1, 2, or 3) G^{C}, OH, C₃₋₆ cycloalkyl optionally substituted with 1-5 (e.g., 1, 2, or 3) G^{C}, 4-6 membered heterocyclyl having 1-3 ring heteroatoms, each independently selected from N, O, and S, which is optionally substituted with 1-5 (e.g., 1, 2, or 3) G^{C}, NH₂, NH (C₁₋₄ alkyl), N(C₁₋₄ alkyl) (C₁₋₄ alkyl), C₁₋₄ alkoxy optionally substituted with 1-5 (e.g., 1, 2, or 3) G^{C}, C₃₋₆ cycloalkoxy optionally substituted with 1-5 (e.g., 1, 2, or 3) G^{C}, or 4-6 membered heterocycloalkoxy optionally substituted with 1-5 (e.g., 1, 2, or 3) G^{c},
wherein G^{C} at each occurrence is independently F, OH, C₁₋₄ alkyl optionally substituted with 1-3 F, or C₁₋₄ alkoxy optionally substituted with 1-3 F,
or R³ at each occurrence is independently F, Cl, Br, CN, C₁₋₄ alkyl optionally substituted with 1-5 (e.g., 1, 2, or 3) G^{C3}, C₂₋₄ alkenyl optionally substituted with 1-5 (e.g., 1, 2, or 3) G^{C3}, C₂₋₄ alkynyl optionally substituted with 1-5 (e.g., 1, 2, or 3) G^{C3}, OH, C₃₋₆ cycloalkyl optionally substituted with 1-5 (e.g., 1, 2, or 3) G^{C}3, 4-6 membered heterocyclyl having 1-3 ring heteroatoms, each independently selected from N, O, and S, which is optionally substituted with 1-5 (e.g., 1, 2, or 3) G^{C}, NH₂, NH (C₁₋₄ alkyl), N(C₁₋₄ alkyl) (C₁₋₄ alkyl), C₁₋₄ alkoxy optionally substituted with 1-5 (e.g., 1, 2, or 3) G^{C3}, C₃₋₆ cycloalkoxy optionally substituted with 1-5 (e.g., 1, 2, or 3) G^{C}3, 4-6 membered heterocycloalkoxy optionally substituted with 1-5 (e.g., 1, 2, or 3) G^{C3}, or a 5 or 6-membered heteroaryl optionally substituted with 1-5 (e.g., 1, 2, or 3) G^{C3};
wherein G^{C3} at each occurrence is independently is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F, or in Formula I-A-1 or I-A-3, one instance of R³ and L¹, together with the intervening atoms, are joined to form an optionally substituted 5-7 membered ring structure.

For example, in some embodiments, R³ at each occurrence can be independently F; Cl; Br; CN; C₁₋₄ alkyl optionally substituted with 1-3 F; OH; C₃₋₆ cycloalkyl optionally substituted with 1-3 G^{C}1; 4-6 membered monocyclic heterocyclyl having 1-2 ring heteroatoms, each independently selected from N, O, and S, which is optionally substituted with 1-3 G^{C1}; or C₁₋₄ alkoxy optionally substituted with 1-3 F; wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F. In some embodiments, R³ at each occurrence can be independently F, Cl, C₁₋₄ alkyl optionally substituted with 1-3 F, cyclopropyl or cyclobutyl.

In some embodiments according to Formula I-A-1 to I-A-3, j is 0.

In some embodiments according to Formula I-A-1 to I-A-3, j is 1 and R³ is defined herein. In some embodiments, when j is 1, R³ is ortho to the carbonyl group in Formula I-A-1 to I-A-3, respectively. In some specific embodiments, when j is 1, R³ is F, Cl, C₁₋₄ alkyl optionally substituted with 1-3 F, cyclopropyl or cyclobutyl. In some specific embodiments, when j is 1, R³ is

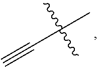 [SMILES: *C#C]

### CN,

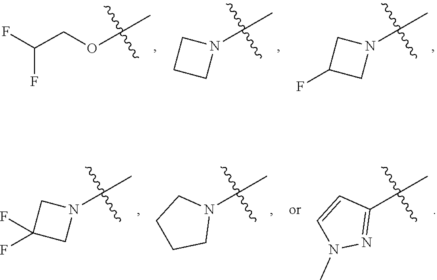

In some embodiments, in Formula I-A-1 or I-A-3, one instance of R³ and L¹, together with the intervening atoms, can be joined to form an optionally substituted 5-7 membered ring structure, typically a 5-7 membered carbocyclic or heterocyclic ring.

In some embodiments, the compound of Formula I is characterized as having Formula I-A-1, wherein one instance of R³ and L¹, together with the intervening atoms, are joined to form an optionally substituted 5-7 membered ring structure such as a 5 or 6 membered ring structure containing 1 or 2 ring heteroatoms, each independently selected from N, O, and S.

For example, in some embodiments, the compound of Formula I can be characterized as having Formula I-A-1-a,

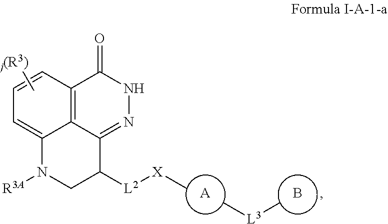

wherein:
the variables R³, L², L³, X, ring A, and ring B include any of those described herein in any combinations,
j is 0, 1, or 2, and
R^{3A} is hydrogen, C₁₋₄ alkyl optionally substituted with 1-5 (e.g., 1, 2, or 3) G^{C}, C₃₋₆ cycloalkyl optionally substituted with 1-5 (e.g., 1, 2, or 3) G^{C}, or 4-6 membered heterocyclyl having 1-3 ring heteroatoms, each independently selected from N, O, and S, which is optionally substituted with 1-5 (e.g., 1, 2, or 3) G^{c},
wherein G^{C} at each occurrence is independently F, OH, C₁₋₄ alkyl optionally substituted with 1-3 F, or C₁₋₄ alkoxy optionally substituted with 1-3 F. In some embodiments, R^{3A} is hydrogen, C₁₋₄ alkyl optionally substituted with 1-3 F, or C₃₋₆ cycloalkyl.

In some embodiments according to Formula I-A-1-a, j is 0.

In some embodiments according to Formula I-A-1-a, j is 1 and R³ is defined herein. In some specific embodiments according to Formula I-A-1-a, when j is 1, R³ is F, Cl, C₁₋₄ alkyl optionally substituted with 1-3 F, cyclopropyl or cyclobutyl. In some specific embodiments according to Formula I-A-1-a, when j is 1, R³ is

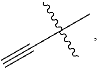 [SMILES: *C#C]

### CN,

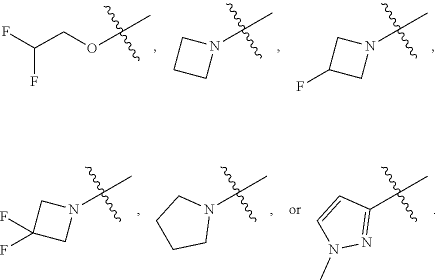

Some embodiments of the present disclosure are directed to compounds of Formula I-B as described herein. In some embodiments, in Formula I (e.g., any of the applicable sub-formulae as described herein, such as I-1, I-2, I-D-1, I-D-2, I-D-3, I-D-1-a, I-D-2-a, or I-D-3-a), R² and L¹, together with the intervening atoms, are joined to form an optionally substituted 5-7 membered heterocyclyl having 1 or 2 ring heteroatoms, each independently selected from O, N, and S. Thus, in such embodiments, ring D in Formula I-B or as applicable in Formula I-A-a, can be an optionally substituted 5-7 membered heterocyclyl having 1 or 2 ring heteroatoms, each independently selected from O, N, and S. In some embodiments, the 5-7 membered heterocyclyl has one ring heteroatom selected from N, S, and O. In some embodiments, the 5-7 membered heterocyclyl has only one ring heteroatom, which is O or N. When substituted, the 5-7 membered heterocyclyl is typically substituted with one or more (e.g., 1-5 or 1-3) substituents independently selected from (1) halo (preferably, F) or CN, (2) OH, (3) NG³G⁴, (4) oxo, (5) G⁵, (6) OG⁵, (7) (C₁₋₄ alkylene)-G⁵, and (8) (C₁₋₄ heteroalkylene)-G⁵,
wherein:
G³ and G⁴ are independently hydrogen or G⁵,
G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii) 4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B},
wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C}; and G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

In some embodiments, when substituted, the 5-7 membered heterocyclyl is substituted with one or more (e.g., 1-5 or 1-3) substituents independently selected from (1) F; (2) oxo; (3) G⁵; (4) (C₁₋₄ alkylene)-G⁵, and (6) (C₁₋₄ heteroalkylene)-G⁵, wherein G⁵ is defined herein. For example, in some embodiments, G⁵ at each occurrence is independently (i) C₁₋₄ alkyl optionally substituted with 1-5 (e.g., 1, 2, or 3) G^{A1}, (ii) C₃₋₆ cycloalkyl optionally substituted with 1-5 (e.g., 1, 2, or 3) G^{A1}, (iii)4-6 membered monocyclic heterocyclyl having 1-2 ring heteroatoms, each independently selected from N, O, and S, which is optionally substituted with 1-5 (e.g., 1, 2, or 3) G^{A1}, (iv) phenyl optionally substituted with 1-5 (e.g., 1, 2, or 3) G^{B1}, or (v)5 or 6 membered heteroaryl having 1-3 ring heteroatoms, which is optionally substituted with 1-5 (e.g., 1, 2, or 3) G^{B1}, wherein
G^{A1} at each occurrence is independently F; oxo; C₁₋₄ alkyl optionally substituted with 1-3 F; OH; NH₂; NH (C₁₋₄ alkyl); N(C₁₋₄ alkyl) (C₁₋₄ alkyl); or C₁₋₄ alkoxy optionally substituted with 1-3 F; and
G^{B1} at each occurrence is independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F;
wherein GCI at each occurrence is independently F; OH; C₁₋₃ alkyl (preferably methyl) optionally substituted with 1-3 F; or C₁₋₃ alkoxy (preferably methoxy) optionally substituted with 1-3 F.

In some embodiments, G⁵ at each occurrence is independently (i) C₁₋₄ alkyl optionally substituted with 1-5 (e.g., 1, 2, or 3) G^{A2}, or (ii) C₃₋₆ cycloalkyl optionally substituted with 1-5 (e.g., 1, 2, or 3) G^{A2}, wherein
G^{A2} at each occurrence is independently F; oxo; C₁₋₃ alkyl (preferably methyl) optionally substituted with 1-3 F; OH; NH₂; NH (C₁₋₃ alkyl), preferably, NHCH₃; N(C₁₋₃ alkyl) (C₁₋₃ alkyl), preferably, N(CH₃)₂; or C₁₋₃ alkoxy (preferably methoxy) optionally substituted with 1-3 F.

In some embodiments, when substituted, the 5-7 membered heterocyclyl is substituted with one or more (e.g., 1-5 or 1-3) substituents independently selected from F, C₁₋₄ alkyl optionally substituted with 1-5 (e.g., 1, 2, or 3) GP, and phenyl optionally substituted with 1-5 (e.g., 1, 2, or 3) G^{B}, wherein GP at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F; wherein G^{B} is defined herein. For example, in some embodiments, G^{B} at each occurrence is G^{B1}, which is independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F; wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F.

In some specific embodiments, the compound of Formula I can be characterized as having Formula I-B-1, I-B-2, or I-B-3:

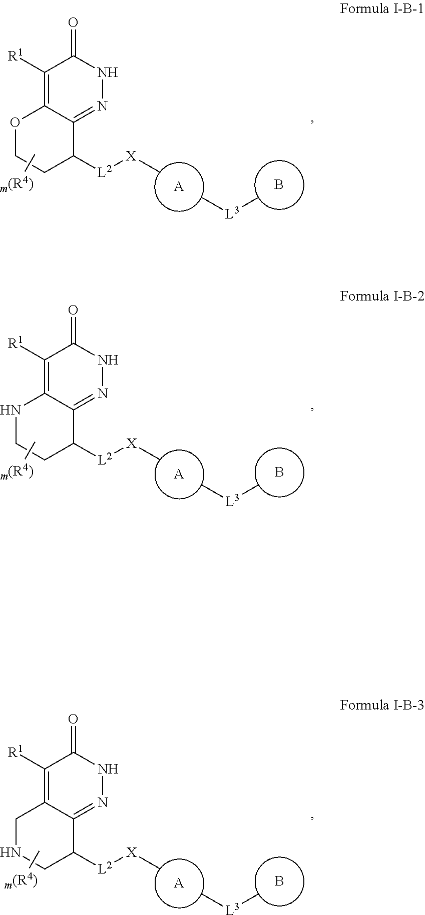

wherein:
the variables R¹, L², L³, X, ring A, and ring B include any of those described herein in any combinations;
m is 0, 1, 2, 3, or 4; and
R⁴ at each occurrence is independently (1) F, (2) OH, (3) NG³G⁴, (4) oxo, (5) G⁵, (6) OG⁵, (7) (C₁₋₄ alkylene)-G⁵, or (8) (C₁₋₄ heteroalkylene)-G⁵, wherein:
G³ and G⁴ are independently hydrogen or G⁵,
G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii) 4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{C};
wherein G^{C} at each occurrence is independently F; OH; C₁₋₄ alkyl optionally substituted with 1-3 F; or C₁₋₄ alkoxy optionally substituted with 1-3 F;
or in Formula I-B-2 or I-B-3, one instance of R⁴ and R¹, together with the intervening atoms, are joined to form an optionally substituted 5-7 membered ring structure;
or two instances of R⁴, together with the intervening atoms, are joined to form an optionally substituted 3-6 membered ring structure;
or one instance of R⁴ and L², together with the intervening atoms, are joined to form an optionally substituted 3-6 membered ring structure.

In some embodiments, G⁵ at each occurrence is independently (i) C₁₋₄ alkyl optionally substituted with 1-5 (e.g., 1, 2, or 3) G^{A1}, (ii) C₃₋₆ cycloalkyl optionally substituted with 1-5 (e.g., 1, 2, or 3) G^{A1}, (iii)4-6 membered monocyclic heterocyclyl having 1-2 ring heteroatoms, each independently selected from N, O, and S, which is optionally substituted with 1-5 (e.g., 1, 2, or 3) G^{A1}, (iv) phenyl optionally substituted with 1-5 (e.g., 1, 2, or 3) G^{B1} or (v)5 or 6 membered heteroaryl having 1-3 ring heteroatoms, which is optionally substituted with 1-5 (e.g., 1, 2, or 3) G^{B1}, wherein
G^{A1} at each occurrence is independently F; oxo; C₁₋₄ alkyl optionally substituted with 1-3 F; OH; NH₂; NH (C₁₋₄ alkyl); N(C₁₋₄ alkyl) (C₁₋₄ alkyl); or C₁₋₄ alkoxy optionally substituted with 1-3 F; and
G^{B1} at each occurrence is independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F;
wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F.

In some embodiments, G⁵ at each occurrence is independently (i) C₁₋₄ alkyl optionally substituted with 1-5 (e.g., 1, 2, or 3) G^{A2}, or (ii) C₃₋₆ cycloalkyl optionally substituted with 1-5 (e.g., 1, 2, or 3) G^{A2}, wherein
G^{A2} at each occurrence is independently F; oxo; C₁₋₃ alkyl (preferably methyl) optionally substituted with 1-3 F; OH; NH₂; NH (C₁₋₃ alkyl), preferably, NHCH₃; N(C₁₋₃ alkyl) (C₁₋₃ alkyl), preferably, N(CH₃)₂; or C₁₋₃ alkoxy (preferably methoxy) optionally substituted with 1-3 F.

In some embodiments, R⁴ at each occurrence is independently C₁₋₄ alkyl optionally substituted with 1-5 (e.g., 1, 2, or 3) GD or phenyl optionally substituted with 1-5 (e.g., 1, 2, or 3) G^{B}, wherein G″ at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F; wherein G^{B} is as defined above. In some specific embodiments, R⁴ at each occurrence is independently methyl, phenyl,

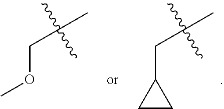

In some embodiments, one instance of R⁴ can be attached to the ring N in Formula I-B-2 or I-B-3, wherein R⁴ is defined herein.

In some embodiments according to Formula I-B-1 to I-B-3, m is 0.

In some embodiments according to Formula I-B-1 to I-B-3, m is 1 and R⁴ is defined herein. For example, in some embodiments, m is 1 and R⁴ is C₁₋₄ alkyl optionally substituted with 1-5 (e.g., 1, 2, or 3) GP or phenyl optionally substituted with 1-5 (e.g., 1, 2, or 3) G^{B}, wherein GD at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F; wherein G^{B} is as defined herein. For example, in some embodiments, m is 1 and R⁴ is C₁₋₄ alkyl optionally substituted with 1-5 (e.g., 1, 2, or 3) GD. In some embodiments, R⁴ is methyl, methoxymethyl, cyclopropyl methyl, etc. In some embodiments, m is 1 and R⁴ is phenyl or phenyl substituted with 1-3 G^{B}, wherein G^{B} is defined herein. In some embodiments, G^{B} is G^{B1} as defined herein and at each occurrence can be independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F; wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F. In some specific embodiments, R⁴ at each occurrence is independently methyl, phenyl,

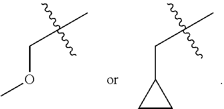

It should be apparent to those skilled in the art that R⁴ may be attached to the ring nitrogen atom in Formula I-B-1 to I-B-3.

In some embodiments, the compound of Formula I-B-1 to I-B-3 can be characterized as having Formula I-B-1-E1, I-B-1-E2, I-B-2-E1, I-B-2-E2, I-B-3-E1, or I-B-3-E2:

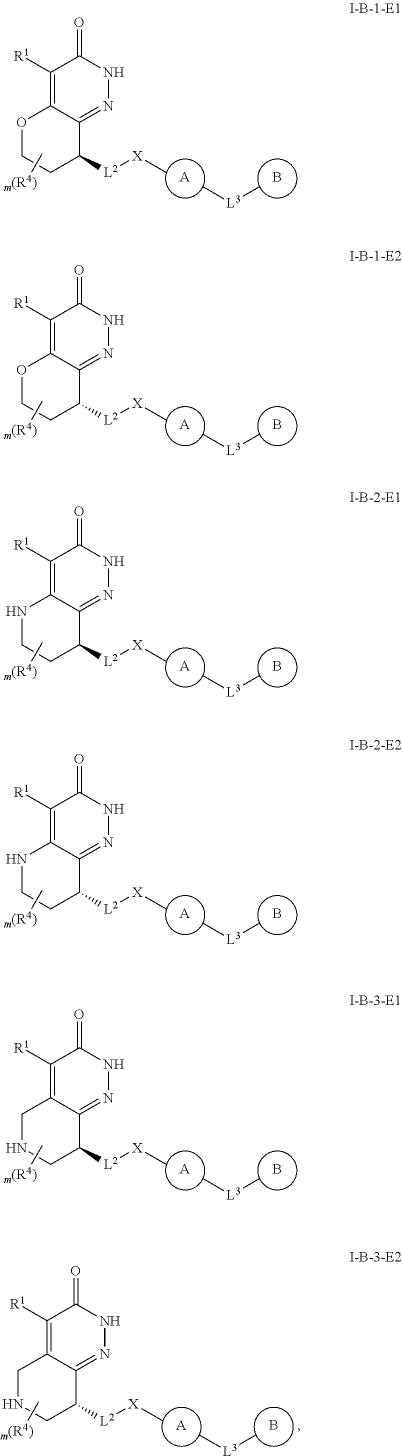

wherein the variables include any of those described herein in connection with the respective Formula I-B-1 to I-B-3 in any combinations. In some embodiments, the compound according to Formula I-B-1-E1, I-B-1-E2, I-B-2-E1, I-B-2-E2, I-B-3-E1, or I-B-3-E2 can exist predominantly as the as-drawn stereoisomer with respect to the drawn stereocenter, for example, free or substantially free of the respective other enantiomer with respect to the drawn stereocenter. However, in some embodiments, the compound according to Formula I-B-1-E1, I-B-1-E2, I-B-2-E1, I-B-2-E2, I-B-3-E1, or I-B-3-E2 can also exist as a mixture in any ratio, such as a racemic mixture, with the respective other enantiomer with respect to the drawn stereocenter.

In some embodiments, in Formula I-B-3, one instance of R⁴ and R¹, together with the intervening atoms, can be joined to form a ring structure of:

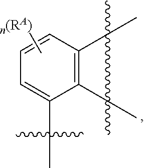

wherein RA is halogen, an optionally substituted C₁₋₄ alkyl, or optionally substituted C₃₋₆ cycloalkyl and n is 0, 1, or 2, wherein the top connecting point of the fragment is to the carbonyl group in Formula I-B-3. As used herein, when it is stated that a substituent or a variable, along with another substituent or a variable, together with the intervening atoms, are joined to form a ring structure, it includes the option that when the pair of substituents or variables are attached to two different atoms, the remaining hydrogen(s) on one or both of the two atoms to which the pair of substituents or variables are attached are eliminated so as to form the designated ring structure. For example, in the case of forming an optionally substituted phenyl from R⁴ and R¹, the additional hydrogen on the atom to which R⁴ is attached is eliminated to form a bond so that the ring structure formed can be a phenyl ring. Other similar situations described herein should be understood similarly.

In some embodiments according to Formula I-B-1 to I-B-3, one instance of R⁴ and L², together with the intervening atoms, can be joined to form an optionally substituted 3-6 membered ring structure. In such embodiments, the 3-6 membered ring structure is typically a non-aromatic ring structure, such as a cycloalkyl, for example cyclopropyl. For example, in some embodiments, the compound of Formula I-B-1-E1 or I-B-1-E2 can be characterized as having one of the following formulae, respectively,

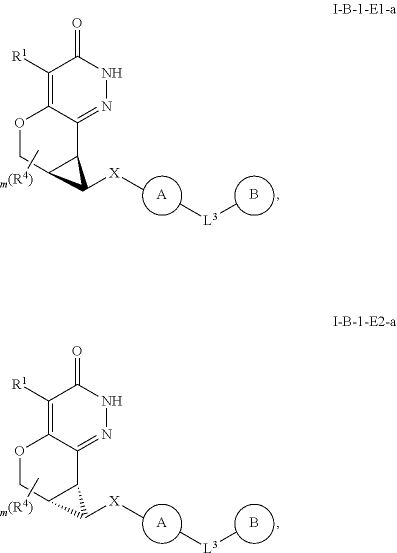

wherein m is 0, 1, 2, or 3, and the variables R¹, R⁴, L³, X, ring A, and ring B include any of those described herein in any combinations.

In some embodiments according to Formula I-B-1 to I-B-3, two instances of R⁴, together with the intervening atom(s), can be joined to form an optionally substituted 3-6 membered ring structure. As will be understood by those skilled in the art, when two R⁴ attached to the same atom are joined to form a ring, then the ring formed is a spiro-ring; when two R⁴ attached to adjacent atoms are joined to form a ring, then the ring formed is a fused ring; and when two R⁴ attached to non-adjacent atoms are joined to form a ring, then the ring system formed is a fused or bridged ring system. Typically, in such embodiments, the 3-6 membered ring structure formed is a non-aromatic ring structure, such as a cycloalkyl or heterocyclyl.

Typically, L¹ and L² in Formula I (e.g., any of the applicable sub-formulae as described herein) are independently null, O, S, S(O), S(O)₂, NR¹⁶, C(O), C(O) O, C(O) NR¹⁶OC(O) NR¹⁶, S(O)₂NR¹⁶, NR¹⁷C(O) NR¹⁶, NR¹⁷S(O)₂NR¹⁶, optionally substituted C₁₋₄ alkylene, optionally substituted C₂₋₄ alkenylene, optionally substituted C₂₋₄ alkynylene, optionally substituted C₁₋₄ heteroalkylene, optionally substituted C₃₋₈ carbocyclylene, optionally substituted 4-10 membered heterocyclylene, optionally substituted phenylene, or optionally substituted 5 or 6 membered heteroarylene, preferably, L¹ and L² are not both null.

In some embodiments, L¹ in Formula I (e.g., any of the applicable sub-formulae as described herein, such as I-1, I-2, I-A, I-A-1, I-A-2, I-A-3, I-D-1, I-D-2, I-D-3, I-D-1-a, I-D-2-a, or I-D-3-a) is an optionally substituted 4-10 membered heterocyclylene having 1-3 ring heteroatoms, each independently selected from O, N, and S. For example, in some embodiments, L¹ in Formula I is an optionally substituted 5 or 6 membered monocyclic heterocyclylene having one or two ring heteroatoms, each independently selected from N, O, and S. Suitable 5 or 6 membered monocyclic heterocyclylenes include any of those described herein. For example, in some embodiments, the 5 or 6 membered monocyclic heterocyclylenes can be a saturated monocyclic ring, such as a pyrrolidine, piperidine, morpholine ring. In some embodiments, L¹ in Formula I is an optionally substituted 6-10 membered fused, spiro, or bridged bicyclic heterocyclylene having one or two ring heteroatoms, each independently selected from N, O, and S. In some embodiments, L¹ in Formula I (e.g., any of the applicable sub-formulae as described herein, such as I-1, I-2, I-A, I-A-1, I-A-2, I-A-3, I-D-1, I-D-2, I-D-3, I-D-1-a, I-D-2-a, or I-D-3-a) is an optionally substituted ring selected from:

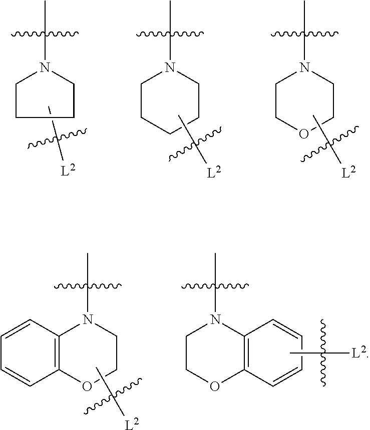

For example, in some specific embodiments, L¹ in Formula I (e.g., any of the applicable sub-formulae as described herein, such as I-1, I-2, I-A, I-A-1, I-A-2, I-A-3, I-D-1, I-D-2, I-D-3, I-D-1-a, I-D-2-a, or I-D-3-a) can be

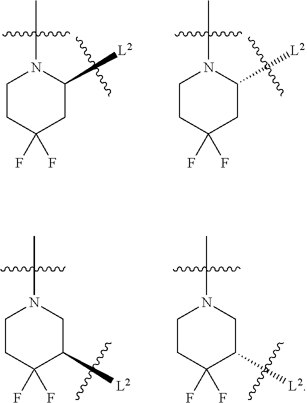

In some specific embodiments, L¹ in Formula I (e.g., any of the applicable sub-formulae as described herein, such as I-1, I-2, I-A, I-A-1, I-A-2, I-A-3, I-D-1, I-D-2, I-D-3, I-D-1-a, I-D-2-a, or I-D-3-a) can be

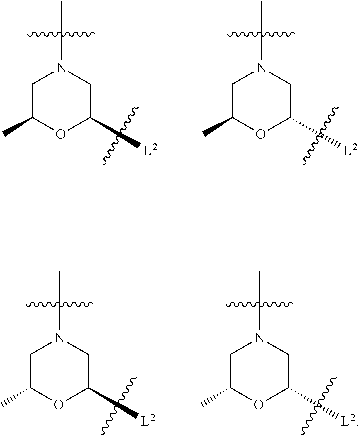

In some specific embodiments, L¹ in Formula I (e.g., any of the applicable sub-formulae as described herein, such as I-1, I-2, I-A, I-A-1, I-A-2, I-A-3, I-D-1, I-D-2, I-D-3, I-D-1-a, I-D-2-a, or I-D-3-a) can be

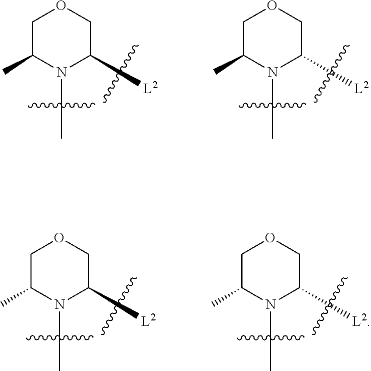

When L¹ in Formula I (e.g., any of the applicable sub-formulae as described herein, such as I-1, I-2, I-A, I-A-1, I-A-2, I-A-3, I-D-1, I-D-2, I-D-3, I-D-1-a, I-D-2-a, or I-D-3-a) is the optionally substituted 4-10 membered heterocyclylene as described herein, the 4-10 membered heterocyclylene can be typically substituted with one or more (e.g., 1-5 or 1-3) substituents independently selected from (1) halo (preferably F or C₁) or CN, (2) OH, (3) NG³G⁴, (4) oxo, (5) G⁵, (6) OG⁵, (7) (C₁₋₄ alkylene)-G⁵, and (8) (C₁₋₄ heteroalkylene)-G⁵, wherein: G³ and G⁴ are independently hydrogen or G⁵, and G⁵ is defined herein. For example, in some embodiments, G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii)4-8 membered heterocyclyl having 1-3 ring heteroatoms, each independently selected from N, O, and S, which is optionally substituted with 1-5 (e.g., 1, 2, or 3) G⁴, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B},
wherein G⁴ at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

In some embodiments, G⁵ at each occurrence is independently (i) C₁₋₄ alkyl optionally substituted with 1-5 (e.g., 1, 2, or 3) G^{A1}, (ii) C₃₋₆ cycloalkyl optionally substituted with 1-5 (e.g., 1, 2, or 3) G^{A1}, (iii)4-6 membered monocyclic heterocyclyl having 1-2 ring heteroatoms, each independently selected from N, O, and S, which is optionally substituted with 1-5 (e.g., 1, 2, or 3) G^{A1}, (iv) phenyl optionally substituted with 1-5 (e.g., 1, 2, or 3) G^{B1} or (v)5 or 6 membered heteroaryl having 1-3 ring heteroatoms, which is optionally substituted with 1-5 (e.g., 1, 2, or 3) G^{B1},
wherein
G^{A1} at each occurrence is independently F; oxo; C₁₋₄ alkyl optionally substituted with 1-3 F; OH; NH₂; NH (C₁₋₄ alkyl); N(C₁₋₄ alkyl) (C₁₋₄ alkyl); or C₁₋₄ alkoxy optionally substituted with 1-3 F; and
G^{B1} at each occurrence is independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F;
wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F.

In some embodiments, G⁵ at each occurrence is independently (i) C₁₋₄ alkyl optionally substituted with 1-5 (e.g., 1, 2, or 3) G^{A2}, or (ii) C₃₋₆ cycloalkyl optionally substituted with 1-5 (e.g., 1, 2, or 3) G^{A2}, wherein
G^{A2} at each occurrence is independently F; oxo; C₁₋₃ alkyl (preferably methyl) optionally substituted with 1-3 F; OH; NH₂; NH (C₁₋₃ alkyl), preferably, NHCH₃; N(C₁₋₃ alkyl) (C₁₋₃ alkyl), preferably, N(CH₃)2; or C₁₋₃ alkoxy (preferably methoxy) optionally substituted with 1-3 F.

In some embodiments, the 4-10 membered heterocyclylene can be substituted with one or more (e.g., 1-5 or 1-3) substituents, each independently halo (preferably F or Cl), CN, C₁₋₄ alkyl optionally substituted with 1-5 (e.g., 1, 2, or 3) GP, or cyclopropyl optionally substituted with 1-5 (e.g., 1, 2, or 3) GP, preferably, each independently F or C₁₋₄ alkyl optionally substituted with 1-5 (e.g., 1, 2, or 3) GP, wherein GD at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F.

In some specific embodiments, the compound of Formula I can be characterized as having Formula I-C-1, I-C-2, or I-C-3:

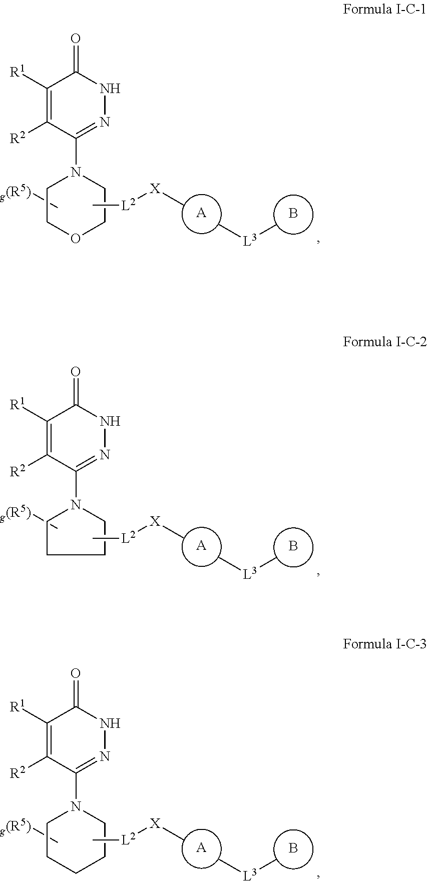

wherein:
the variables R¹, R², L², L³, X, ring A, and ring B include any of those described herein in any combinations;
g is 0, 1, 2, 3, or 4; and
R⁵ at each occurrence is independently selected from (1) halo (preferably F or Cl) or CN, (2) OH, (3) NG³G⁴, (4) oxo, (5) G⁵, (6) OG⁵, (7) (C₁₋₄ alkylene)-G⁵, and (8) (C₁₋₄ heteroalkylene)-G⁵, wherein: G³ and G⁴ are independently hydrogen or G⁵, and G⁵ is defined herein;
or one instance of R⁵ and R², together with the intervening atoms, are joined to form an optionally substituted 5-7 membered ring structure;
or two instances of R⁵, together with the intervening atoms, are joined to form an optionally substituted 5-7 membered ring structure;
or one instance of R⁵ and L², together with the intervening atoms, are joined to form an optionally substituted 5-7 membered ring structure. In some embodiments, G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii)4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B},
wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and
G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

In some embodiments, G⁵ at each occurrence is independently (i) C₁₋₄ alkyl optionally substituted with 1-5 (e.g., 1, 2, or 3) G^{A1}, (ii) C₃₋₆ cycloalkyl optionally substituted with 1-5 (e.g., 1, 2, or 3) G^{A1}, (iii)4-6 membered monocyclic heterocyclyl having 1-2 ring heteroatoms, each independently selected from N, O, and S, which is optionally substituted with 1-5 (e.g., 1, 2, or 3) G^{A1}, (iv) phenyl optionally substituted with 1-5 (e.g., 1, 2, or 3) G^{B1}, or (v)5 or 6 membered heteroaryl having 1-3 ring heteroatoms, which is optionally substituted with 1-5 (e.g., 1, 2, or 3) G^{B1},
wherein
G^{A1} at each occurrence is independently F; oxo; C₁₋₄ alkyl optionally substituted with 1-3 F; OH; NH₂; NH (C₁₋₄ alkyl); N(C₁₋₄ alkyl) (C₁₋₄ alkyl); or C₁₋₄ alkoxy optionally substituted with 1-3 F; and
G^{B1} at each occurrence is independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F;
wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F.

In some embodiments, G⁵ at each occurrence is independently (i) C₁₋₄ alkyl optionally substituted with 1-5 (e.g., 1, 2, or 3) G^{A2}, or (ii) C₃₋₆ cycloalkyl optionally substituted with 1-5 (e.g., 1, 2, or 3) G^{A2}, wherein G^{A2} at each occurrence is independently F; oxo; C₁₋₃ alkyl (preferably methyl) optionally substituted with 1-3 F; OH; NH₂; NH (C₁₋₃ alkyl), preferably, NHCH₃; N(C₁₋₃ alkyl) (C₁₋₃ alkyl), preferably, N(CH₃)₂; or C₁₋₃ alkoxy (preferably methoxy) optionally substituted with 1-3 F.

In some embodiments according to Formula I-C-1 to I-C-3, R⁵ at each occurrence is independently halo (preferably F or Cl), CN, C₁₋₄ alkyl optionally substituted with 1-5 (e.g., 1, 2, or 3) GP, or cyclopropyl optionally substituted with 1-5 (e.g., 1, 2, or 3) G″, preferably, each independently F or C₁₋₄ alkyl optionally substituted with 1-5 (e.g., 1, 2, or 3) GP, wherein G″ at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F. In some embodiments, R⁵ at each occurrence is independently F or C₁₋₄ alkyl such as methyl.

In some embodiments according to Formula I-C-1 to I-C-3, g is 0.

In some embodiments according to Formula I-C-1 to I-C-3, g is 1 or 2, wherein R⁵ is defined herein. For example, in some embodiments, g is 1 or 2, and R⁵ at each occurrence is independently halo (preferably F or Cl), CN, C₁₋₄ alkyl optionally substituted with 1-5 (e.g., 1, 2, or 3) G″, or cyclopropyl optionally substituted with 1-5 (e.g., 1, 2, or 3) G″, preferably, each independently F or C₁₋₄ alkyl optionally substituted with 1-5 (e.g., 1, 2, or 3) G″, wherein GD at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F. In some embodiments, R⁵ at each occurrence is independently F or C₁₋₄ alkyl such as methyl.

The L²-X-(Ring A)-L³-(Ring B) moiety in Formula I-C-1 can be attached to the morpholine ring either next to the ring nitrogen or next to the ring oxygen. For example, in some embodiments, the compound of Formula I-C-1 can have a formula of I-C-1-E1, I-C-1-E2, I-C-1-E3, or I-C-1-E4:

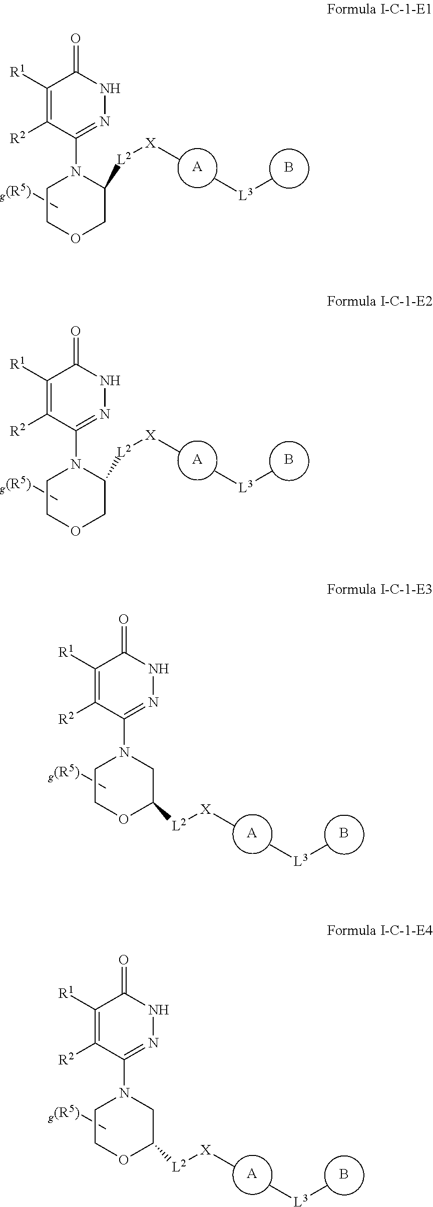

wherein the variables in the formulae include any of those described herein for Formula I-C-1 in any combinations. In some embodiments, the compound according to Formulae I-C-1-E1 to I-C-1-E4 can exist predominantly as the as-drawn stereoisomer, which can for example, be free or substantially free of the respective other enantiomer with respect to the drawn stereocenter. However, in some embodiments, the compound according to Formulae I-C-1-E1 to I-C-1-E4 can also exist as a mixture in any ratio, such as a racemic mixture, with the respective other enantiomer with respect to the drawn stereocenter.

Similarly, the L²-X-(Ring A)-L³-(Ring B) moiety in Formula I-C-2 can be attached to the pyrrolidine ring either next to the ring nitrogen or not. For example, in some embodiments, the compound of Formula I-C-2 can have a formula of I-C-2-E1, I-C-2-E2, I-C-2-E3, or I-C-2-E4:

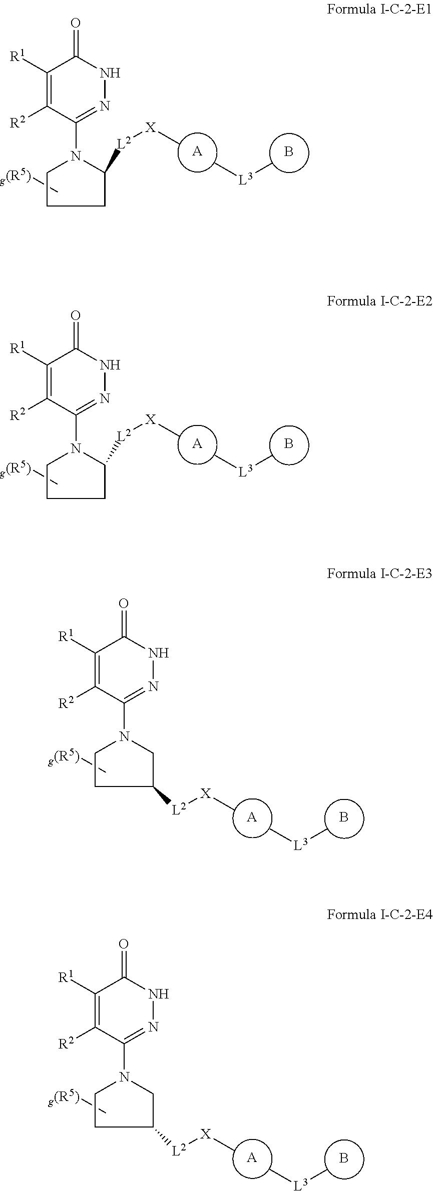

wherein the variables in the formulae include any of those described herein for Formula I-C-2 in any combinations. In some embodiments, the compound according to Formulae I-C-2-E1 to I-C-2-E4 can exist predominantly as the as-drawn stereoisomer, which can for example be free or substantially free of the respective other enantiomer with respect to the drawn stereocenter. However, in some embodiments, the compound according to Formulae I-C-2-E1 to I-C-2-E4 can also exist as a mixture in any ratio, such as a racemic mixture, with the respective other enantiomer with respect to the drawn stereocenter.

The L²-X-(Ring A)-L³-(Ring B) moiety in Formula I-C-3 can be attached to the piperidine ring either next to the nitrogen or not. For example, in some embodiments, the compound of Formula I-C-3 can have a formula of I-C-3-E1, I-C-3-E2, I-C-3-E3, or I-C-3-E4:

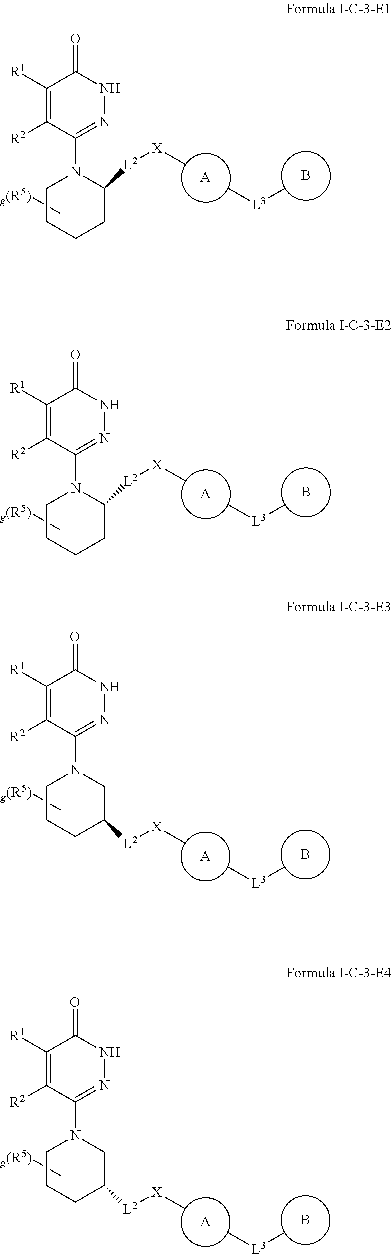

wherein the variables in the formulae include any of those described herein for Formula I-C-3 in any combinations. In some embodiments, the compound according to Formulae I-C-3-E1 to I-C-3-E4 can exist predominantly as the as-drawn stereoisomer, which can for example be free or substantially free of the respective other enantiomer with respect to the drawn stereocenter. However, in some embodiments, the compound according to Formulae I-C-3-E1 to I-C-3-E4 can also exist as a mixture in any ratio, such as a racemic mixture, with the respective other enantiomer with respect to the drawn stereocenter.

In some embodiments according to Formula I-C-1 to I-C-3, one instance of R⁵ and R², together with the intervening atoms, are joined to form an optionally substituted 5-7 membered ring structure. For example, in some embodiments, one instance of R⁵ is attached next to the nitrogen atom and together with R² and the intervening atoms, form an optionally substituted 5-7 membered ring structure.

In some embodiments according to Formula I-C-1 to I-C-3, two instances of R⁵, together with the intervening atoms, can be joined to form an optionally substituted 5-7 membered ring structure, such as an optionally substituted phenyl or optionally substituted pyridyl. For example, in some embodiments, the compound of Formula I-C-1 (e.g., I-C-1-E1, I-C-1-E2, I-C-1-E3, or I-C-1-E4) can be characterized as having a Formula I-C-1-a:

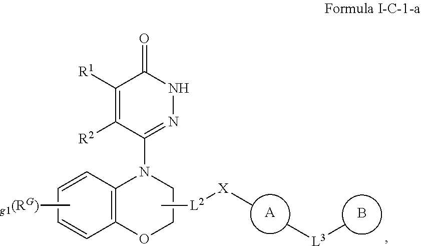

wherein:
the variables R¹, R², L², L³, X, ring A, and ring B include any of those described herein in any combinations, including any of those shown for Formula I-C-1-E1, I-C-1-E2, I-C-1-E3, or I-C-1-E4;
R^{G} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F;
or R^{G} at each occurrence is independently halo (preferably, F, Cl, or Br); CN; OH; NH₂; G^{A4}; OG^{A4}, NHG^{A4}; N(C₁₋₄ alkyl) (G⁴⁴); COG^{A4}; SO₂G^{A4}; CONHG^{A4}; CON(C₁₋₄ alkyl) (G^{A4}); NHCOG^{A4}; or N(C₁₋₄ alkyl) COG^{A4};
wherein G^{A4} at each occurrence is independently (1) C₁₋₄ alkyl optionally substituted with 1-5 (e.g., 1, 2, or 3) G^{C4}; (2) C₂₋₄ alkenyl optionally substituted with 1-5 (e.g., 1, 2, or 3) G^{C4}; (3) C₂₋₄ alkynyl optionally substituted with 1-5 (e.g., 1, 2, or 3) G^{C4}; (4) C₁₋₄ heteroalkyl having 1 or 2 heteroatoms independently N, O, or S, wherein the S, if present, is optionally oxidized in the form of SO or SO₂, wherein the C₁₋₄ heteroalkyl is optionally substituted with 1-5 (e.g., 1, 2, or 3) G^{C4}; (6) C₃₋₆ cycloalkyl optionally substituted with 1-5 (e.g., 1, 2, or 3) G^{C4}; (7)4-6 membered heterocyclyl having 1-3 ring heteroatoms, each independently selected from N, O, and S, which is optionally substituted with 1-5 (e.g., 1, 2, or 3) G^{C4}; or (8) phenyl or 5 or 6-membered heteroaryl, each of which is optionally substituted with 1-5 (e.g., 1, 2, or 3) G^{C}4;
wherein G^{C4} at each occurrence is independently (a) halogen (e.g., F, Cl), OH, oxo (as applicable), or CN, (b) C₁₋₄ alkyl optionally substituted with 1-3 F, (c)3-4 membered ring (e.g., cyclopropyl, cyclobutyl, azetidinyl, oxetanyl, etc.) optionally substituted with 1-3 substituents independently F, OH, CN, or methyl, or (d) C₁₋₄ heteroalkyl having 1 or 2 heteroatoms independently O, N, or S, wherein the S, if present, is optionally oxidized in the form of SO or SO₂, wherein the C₁₋₄ heteroalkyl is which is optionally substituted with 1-3 F;
and
g1 is an integer selected from 0, 1, 2, or 3, preferably, 0 or 1. To be clear and as would be understood by those skilled in the art, the R^{G} group(s), if exist, should be attached to the phenyl portion of the bicyclic ring, whereas the residue of -L²-X-(Ring A)-L³-(Ring B) is attached to the oxazine portion of the bicyclic ring.

In some embodiments according to Formula I-C-1-a, g1 is 0.

In some embodiments according to Formula I-C-1-a, g1 is 1, wherein R^{G} is defined herein, preferably, in such case, R^{G} is attached to a position para to the oxygen atom or para to the nitrogen atom, for example, the compound can have a structure according to Formula I-C-1-al or I-C-1-a2:

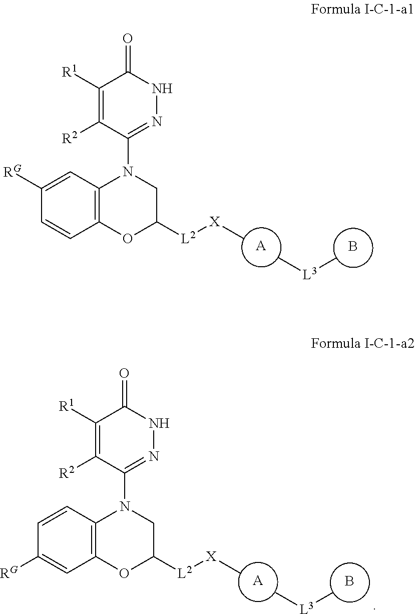

In some embodiments, g1 is 1, and R^{G} is halo (preferably F or Cl), CN, C₁₋₄ alkyl optionally substituted with 1-5 (e.g., 1, 2, or 3) GP, or cyclopropyl optionally substituted with 1-5 (e.g., 1, 2, or 3) GP, preferably, R^{G} is F, Cl, CN, cyclopropyl, or C₁₋₄ alkyl optionally substituted with 1-5 (e.g., 1, 2, or 3) GD, wherein GD at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F. In some embodiments, R^{G} is F, Cl, CN, cyclopropyl, or C₁₋₄ alkyl such as methyl. In some embodiments, R^{G} is C₁₋₄ alkyl optionally substituted with 1-3 F (e.g., CHF₂). In some embodiments, R^{G} is C₂₋₄ alkynyl, such as

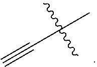 [SMILES: *C#C]

In some embodiments, R^{G} is C₁₋₄ heteroalkyl having 1 or 2 heteroatoms independently N, O, or S, wherein the S, if present, is optionally oxidized in the form of SO or SO₂, wherein the C₁₋₄ heteroalkyl is optionally substituted with 1-5 (e.g., 1, 2, or 3) G^{C4}, for example, R^{G} is

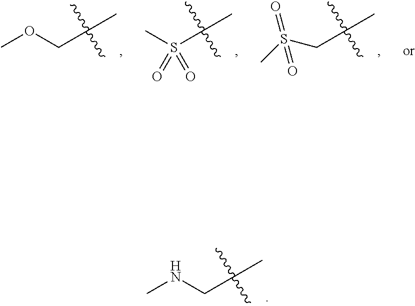

In some embodiments, R^{G} is C₃₋₆ cycloalkyl, such as cyclopropyl, optionally substituted with 1-5 (e.g., 1, 2, or 3) G^{C4}, for example, R^{G} is cyclopropyl or

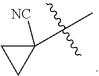 [SMILES: *C1(C#N)CC1]

In some embodiments, R^{G} is 4-6 membered heterocyclyl having 1-3 ring heteroatoms, each independently selected from N, O, and S, which is optionally substituted with 1-5 (e.g., 1, 2, or 3) G^{C4}, such as

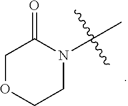 [SMILES: *N1CCOCC1=O]

In some embodiments, R^{G} is 5 or 6-membered heteroaryl, each of which is optionally substituted with 1-5 (e.g., 1, 2, or 3) G^{C4}, such as

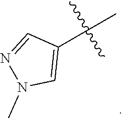 [SMILES: *c1cnn(C)c1]

In some embodiments, R^{G} is CONHG^{A4}, for example,

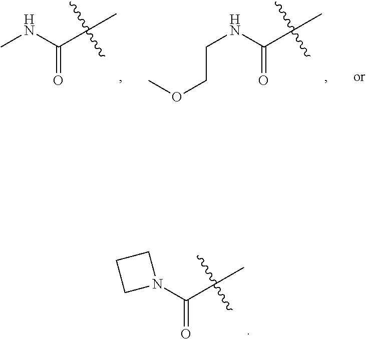

In some embodiments, R^{G} is NHCOG^{A4}, for example,

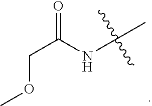 [SMILES: *NC(=O)COC]

In some embodiments, R^{G} at each occurrence is independently F, Cl, CN, C₁₋₄ alkyl optionally substituted with 1-3 F (e.g., CHF₂), cyclopropyl,

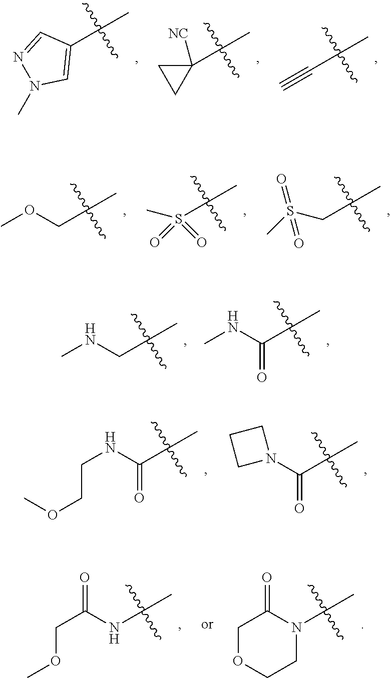

In some embodiments according to Formula I-C-1 to I-C-3, one instance of R⁵ and L², together with the intervening atoms, can be joined to form an optionally substituted 5-7 membered ring structure, such as a cyclopropyl.

In some embodiments, L¹ in Formula I (e.g., any of the applicable sub-formulae as described herein) is null, preferably, when L¹ is null, L² is not also null.

In some embodiments, L¹ in Formula I (e.g., any of the applicable sub-formulae as described herein) is S(O), S(O)₂, C(O), C(O) O, C(O) NR¹⁶, OC(O) NR¹⁶, S(O)₂NR¹⁶, NR¹⁷C(O) NR¹⁶, or NR¹⁷S(O)₂NR¹⁶, wherein R¹⁶ and R¹⁷ are defined herein. For example, in some embodiments, R¹⁶ and R¹⁷ are independently hydrogen or an optionally substituted C₁₋₄ alkyl.

In some embodiments, L¹ in Formula I (e.g., any of the applicable sub-formulae as described herein) is an optionally substituted alkyelene, such as an optionally substituted C₁₋₄ alkylene, which can be straight chained or branched.

In some embodiments, L¹ in Formula I (e.g., any of the applicable sub-formulae as described herein) is an optionally substituted alkenylene, such as an optionally substituted C₂₋₄ alkenylene, which can be straight chained or branched.

In some embodiments, L¹ in Formula I (e.g., any of the applicable sub-formulae as described herein) is an optionally substituted alkynylene, such as an optionally substituted C₂₋₄ alkynylene, which can be straight chained or branched.

In some embodiments, L¹ in Formula I (e.g., any of the applicable sub-formulae as described herein) is an optionally substituted heteroalkylene, such as an optionally substituted C₁₋₄ heteroalkylene, which can be straight chained or branched.

In some embodiments, L¹ in Formula I (e.g., any of the applicable sub-formulae as described herein) is an optionally substituted cycloalkylene, such as an optionally substituted C₃₋₆ cycloalkylene.

In some embodiments, L¹ in Formula I (e.g., any of the applicable sub-formulae as described herein) is an optionally substituted phenylene.

In some embodiments, L¹ in Formula I (e.g., any of the applicable sub-formulae as described herein) is an optionally substituted heteroarylene, such as an optionally substituted 5 or 6 membered heteroarylene having 1-3 ring heteroatoms, each independently selected from O, N, and S.

In some embodiments, L¹ in Formula I (e.g., any of the applicable sub-formulae as described herein) can be O, S, or NR¹⁶, wherein R¹⁶ is hydrogen or an optionally substituted C₁₋₄ alkyl, e.g., methyl.

In some specific embodiments, L¹ in Formula I (e.g., any of the applicable sub-formulae as described herein) can be O.

In some embodiments, L² in Formula I (e.g., any of the applicable sub-formulae as described herein, such as I-1, 1-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4) is null, preferably, when L² is null, L¹ is not also null.

In some embodiments, L² in Formula I (e.g., any of the applicable subformulae herein) is S(O), S(O)₂, C(O), C(O) O, C(O) NR¹⁶, OC(O) NR¹⁶, S(O)₂NR¹⁶, NR¹⁷C(O) NR¹⁶, or NR¹⁷S(O)₂NR¹⁶, wherein R¹⁶ and R¹⁷ are defined herein. For example, in some embodiments, R¹⁶ and R¹⁷ are independently hydrogen or an optionally substituted C₁₋₄ alkyl.

In some embodiments, L² in Formula I (e.g., any of the applicable subformulae herein) is an optionally substituted alkyelene, such as an optionally substituted C₁₋₄ alkylene, which can be straight chained or branched, such as methylene, ethylene, etc.

In some embodiments, L² in Formula I (e.g., any of the applicable subformulae herein) is an optionally substituted alkenylene, such as an optionally substituted C₂₋₄ alkenylene, which can be straight chained or branched.

In some embodiments, L² in Formula I (e.g., any of the applicable subformulae herein) is an optionally substituted alkynylene, such as an optionally substituted C₂₋₄ alkynylene, which can be straight chained or branched.

In some embodiments, L² in Formula I (e.g., any of the applicable subformulae herein) is an optionally substituted heteroalkylene, such as an optionally substituted C₁₋₄ heteroalkylene, which can be straight chained or branched.

In some embodiments, L² in Formula I (e.g., any of the applicable subformulae herein) is an optionally substituted cycloalkylene, such as an optionally substituted C₃₋₆ cycloalkylene.

In some embodiments, L² in Formula I (e.g., any of the applicable subformulae herein) is an optionally substituted heterocyclylene, such as an optionally substituted 4-10 membered (e.g., 3-8 or 5-8 membered) heterocyclylene having 1-3 ring heteroatoms, each independently selected from O, N, and S.

In some embodiments, L² in Formula I (e.g., any of the applicable subformulae herein) is an optionally substituted phenylene. When substituted, the phenylene can be typically substituted with 1-5 (e.g., 1, 2, or 3) G^{B},
wherein G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

In some embodiments, the phenylene can be typically substituted with 1-5 (e.g., 1, 2, or 3) G^{B1} wherein G^{B1} at each occurrence is independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F, wherein G^{C1} at each occurrence is independently F; OH; C₁₋₃ alkyl (preferably methyl) optionally substituted with 1-3 F; or C₁₋₃ alkoxy (preferably methoxy) optionally substituted with 1-3 F.

In some specific embodiments, L² in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4) can be a phenylene selected from:

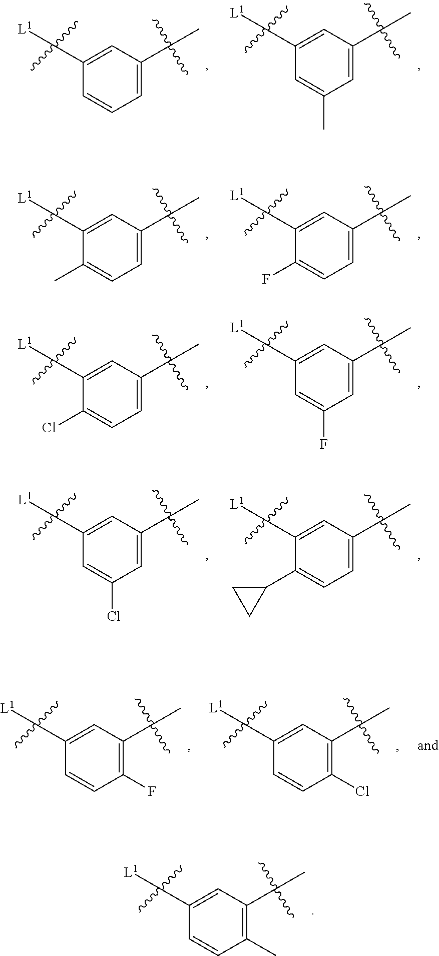

In some embodiments, L² in Formula I (e.g., any of the applicable subformulae herein) is an optionally substituted heteroarylene, such as an optionally substituted 5 or 6 membered heteroarylene having 1-3 ring heteroatoms, each independently selected from O, N, and S. When substituted, the heteroarylene can be typically substituted with 1-5 (e.g., 1, 2, or 3) G^{B},
wherein G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{C};
wherein G^{C} at each occurrence is independently F; OH; C₁₋₄ alkyl optionally substituted with 1-3 F; or C₁₋₄ alkoxy optionally substituted with 1-3 F.

In some embodiments, the heteroarylene can be substituted with 1-5 (e.g., 1, 2, or 3) G^{B1}, wherein G^{B1} at each occurrence is independently F; Cl; Br; CN; C₁₋₄ alkyl optionally substituted with 1-3 F; OH; C₃₋₆ cycloalkyl; 4-6 membered monocyclic heterocyclyl having 1-2 ring heteroatoms, each independently selected from N, O, and S, which is optionally substituted with 1-5 (e.g., 1, 2, or 3) G^{C1}; NH₂; NH (C₁₋₄ alkyl); N(C₁₋₄ alkyl) (C₁₋₄ alkyl); or C₁₋₄ alkoxy optionally substituted with 1-3 F, wherein GCI at each occurrence is independently F; OH; C₁₋₃ alkyl (preferably methyl) optionally substituted with 1-3 F; or C₁₋₃ alkoxy (preferably methoxy) optionally substituted with 1-3 F.

In some specific embodiments, L² in Formula I (e.g., any of the applicable subformulae herein) can be

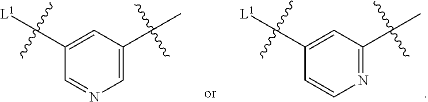

In some embodiments, L² in Formula I (e.g., any of the applicable subformulae herein) can be O.

In some embodiments, L² in Formula I (e.g., any of the applicable subformulae herein) can be a C₁₋₄ heteroalkylene having 1 or 2 heteroatoms, each independently selected from O, S, and N. For example, in some embodiments, L² in Formula I can be a C₁₋₄ heteroalkylene having 1 heteroatom, which is O. In some specific embodiments, L² in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4) can be

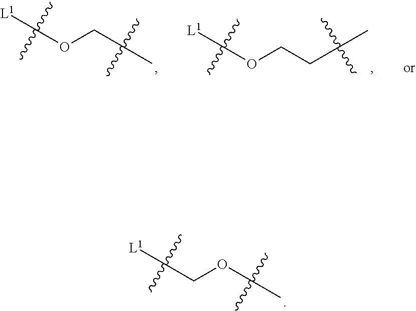

In some embodiments, the compound of Formula I can be characterized as having a Formula I-D-1, I-D-2, or I-D-3:

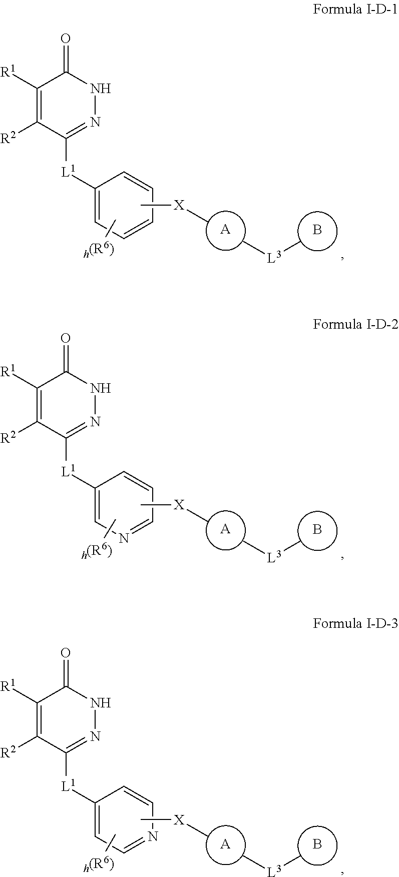

wherein:
the variables R¹, R², L¹, L³, X, ring A, and ring B include any of those described herein in any combinations;
h is 0, 1, or 2, and
R⁶ at each occurrence is independently F, Cl, Br, CN, C₁₋₄ alkyl optionally substituted with 1-5 (e.g., 1, 2, or 3) G^{C}, OH, cyclopropyl, cyclobutyl, 4-6 membered heterocyclyl having 1-3 ring heteroatoms independently selected from N, O, and S, which is optionally substituted with 1-5 (e.g., 1, 2, or 3) G^{C}, NH₂, NH (C₁₋₄ alkyl), N(C₁₋₄ alkyl) (C₁₋₄ alkyl), or C₁₋₄ alkoxy optionally substituted with 1-5 (e.g., 1, 2, or 3) G^{c},
wherein G^{C} at each occurrence is independently F, OH, C₁₋₄ alkyl optionally substituted with 1-3 F, or C₁₋₄ alkoxy optionally substituted with 1-3 F;
or one instance of R⁶ and L¹, together with the intervening atoms, are joined to form an optionally substituted 5-7 membered ring structure.

In some embodiments, R⁶ at each occurrence is F, Cl, C₁₋₄ alkyl optionally substituted with 1-3 F, or cyclopropyl.

Typically, in Formula I-D-1 to I-D-3, L¹ is O, S, NH, or NCH₃. In some specific embodiments, in Formula I-D-1 to I-D-3, L¹ is O.

In some embodiments according to Formula I-D-1 to I-D-3, h is 0.

In some embodiments according to Formula I-D-1 to I-D-3, h is 1 and R⁶ is defined herein. For example, in some embodiments, R⁶ at each occurrence is F, Cl, C₁₋₄ alkyl optionally substituted with 1-3 F, or cyclopropyl.

In embodiments according to Formula I-D-1 to I-D-3, the moiety X-(Ring A)-L³-(Ring B) is typically attached to the phenylene or pyridylene at a meta-position to L¹. For example, in some embodiments, the compound of Formula I-D-1 to I-D-3 can be characterized as having one of the following formulae, respectively:

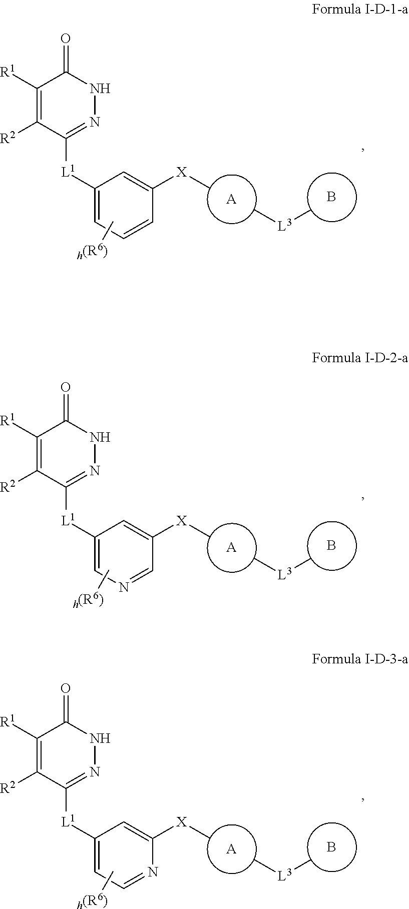

wherein the variables R¹, R², R⁶, h, L¹, L³, X, ring A, and ring B include any of those described herein for Formula I-D-1 to I-D-3 in any combinations. For example, in some embodiments, L¹ is O, and the compound can be characterized as having one of the following formulae:

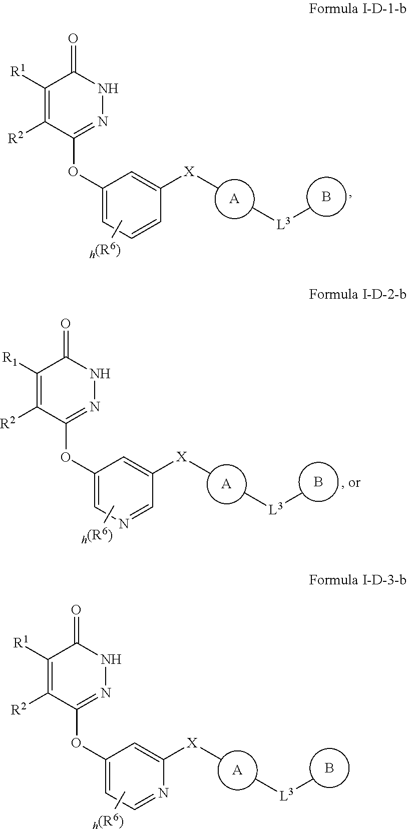

wherein the variables R¹, R², R⁶, h, L³, X, ring A, and ring B include any of those described herein for Formula I-D-1 to I-D-3 in any combinations.

In some embodiments according to Formula I-D-1 to I-D-3, one instance of R⁶ and L¹, together with the intervening atoms, are joined to form an optionally substituted 5-7 membered ring structure. For example, in some embodiments, the compound of Formula I-D-1 can be characterized as having the following formula, I-D-1-c:

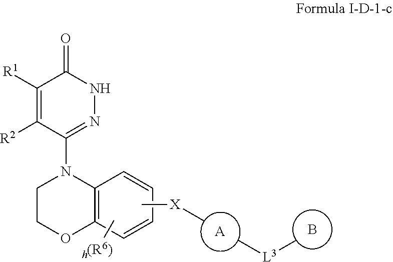

wherein his 0 or 1, and the variables R¹, R², R⁶, L³, X, ring A, and ring B include any of those described herein in any combinations. As would be understood by those skilled in the art, in Formula I-D-1-c, R⁶ and -X-(Ring A)-L³-(Ring B) are both attaching to the phenyl ring.

Typically, in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), X is C(O).

However, in some embodiments, X in Formula I (e.g., any of the applicable sub-formulae as described herein) can also be G¹-C(O)-G², wherein G¹ and G² are defined herein. For example, in some embodiments, G¹ and G² are each independently null, O, NH, optionally substituted C₁₋₄ alkylene, such as methylene, or optionally substituted C₁₋₄ heteroalkylene. In some embodiments, G¹ and G² are joined to form an optionally substituted 4-7 membered ring, typically has one or two ring heteroatoms, each independently O or N, for example, a lactam ring or imidazolidinone ring. For example, in some embodiments, X in Formula I (e.g., any of the applicable sub-formulae as described herein) can also be

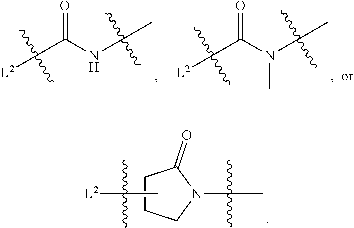

In some embodiments, X in Formula I (e.g., any of the applicable sub-formulae as described herein) can also be G¹-S(O)₂-G², wherein G¹ and G² are defined herein. For example, in some embodiments, G¹ and G² are each independently null, O, NH, optionally substituted C₁₋₄ alkylene, such as methylene, or optionally substituted C₁₋₄ heteroalkylene. In some embodiments, G¹ and G² are joined to form an optionally substituted 4-7 membered ring, typically in addition to the S atom from the SO₂ group, has one or two ring heteroatoms, each independently O or N.

In some embodiments, X in Formula I (e.g., any of the applicable sub-formulae as described herein) can also be S(O) or S(O)₂.

In some embodiments, X in Formula I (e.g., any of the applicable sub-formulae as described herein) can be or S(O)₂.

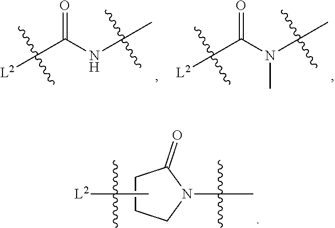

Ring A in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) is typically an optionally substituted 4-7 membered monocyclic heterocyclyl having 1 or 2 ring heteroatoms independently selected from N, O, and S, preferably, at least one of the ring heteroatoms is N. For example, in some embodiments, Ring A in Formula I can be a saturated 4 or 6 membered heterocyclic ring having one or two ring heteroatoms, such as one or two ring nitrogens, such as a pyrrolidine ring or a piperazine ring, which is optionally substituted. When substituted, the 4-7 membered monocyclic heterocyclyl is typically substituted with 1-5 (e.g., 1, 2, or 3) G^{A}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C}; wherein G^{C} at each occurrence is independently F, OH, C₁₋₄ alkyl optionally substituted with 1-3 F, or C₁₋₄ alkoxy optionally substituted with 1-3 F. In some embodiments, when substituted, the 4-7 membered monocyclic heterocyclyl is substituted with 1-5 (e.g., 1, 2, or 3) G^{A}, wherein G^{A} at each occurrence is independently F; oxo; methyl; OH; NH₂; NH (CH₃); N(CH₃)₂; or methoxy. In some embodiments, the 4-7 membered monocyclic heterocyclyl is not substituted.

In some embodiments, Ring A in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) is an optionally substituted 6-10 membered fused, spiro, or bridged bicyclic heterocyclyl having 1 or 2 ring heteroatoms, each independently selected from N, O, and S, preferably, at least one of the ring heteroatoms is N. When substituted, the 6-10 membered fused, spiro, or bridged bicyclic heterocyclyl is substituted with 1-5 (e.g., 1, 2, or 3) G^{A}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C}; wherein G^{C} at each occurrence is independently F, OH, C₁₋₄ alkyl optionally substituted with 1-3 F, or C₁₋₄ alkoxy optionally substituted with 1-3 F. In some embodiments, when substituted, the 6-10 membered fused, spiro, or bridged bicyclic heterocyclyl is substituted with 1-5 (e.g., 1, 2, or 3) G^{A}, wherein G^{A} at each occurrence is independently F; oxo; methyl; OH; NH₂; NH (CH₃); N(CH₃)₂; or methoxy. In some embodiments, the 6-10 membered fused, spiro, or bridged bicyclic heterocyclyl is not substituted.

In some specific embodiments, Ring A in Formula I (e.g., any of the applicable sub-formulae as described herein) is

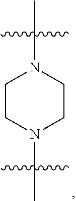 [SMILES: *N1CCN(*)CC1]

which is optionally substituted. In some specific embodiments, Ring A in Formula I (e.g., any of the applicable sub-formulae as described herein) is

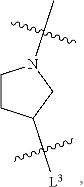

which is optionally substituted. When substituted, the piperazine or pyrrolidine is typically substituted with 1-5 (e.g., 1, 2, or 3) G^{A}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C}; wherein G^{C} at each occurrence is independently F, OH, C₁₋₄ alkyl optionally substituted with 1-3 F, or C₁₋₄ alkoxy optionally substituted with 1-3 F. For example, in some embodiments, the piperazine or pyrrolidine can be substituted with 1-5 (e.g., 1, 2, or 3) G^{A}, wherein G^{A} at each occurrence is independently F; oxo; methyl; OH; NH₂; NH (CH₃); N(CH₃)₂; or methoxy. In some embodiments, when substituted, two substituents of the piperazine or pyrrolidine, together with the intervening atom(s), are joined to form a 3-4 membered ring, such as cyclopropyl, and the piperazine or pyrrolidine is optionally further substituted with 1-3 G^{A}, wherein G^{A} is defined above, e.g., ring A can be

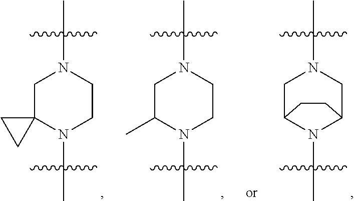

wherein either the top or the bottom attaching point can be connected to L³-Ring B, preferably, the bottom attaching point is connected to L³-Ring B. In some embodiments, the piperazine or pyrrolidine is not substituted.

In some specific embodiments, Ring A in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) is

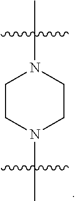 [SMILES: *N1CCN(*)CC1]

Typically, in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), L³ is null. Typically, in such embodiments, Ring A connects to Ring B through a ring nitrogen atom.

In some embodiments, Ring A can also connect to Ring B through L³, which can be O, NH, or N(C₁₋₄ alkyl), provided that L³ does not connect to a ring heteroatom of ring A or ring B.

Ring B in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) is typically an optionally substituted 5 or 6 membered heteroaryl having 1-3 ring heteroatoms independently selected from N, O, and S. For example, in some embodiments, Ring B in Formula I is an optionally substituted pyridine, pyrazine, thiazole, thiadiazole, or pyrimidine. In some embodiments, when substituted, the 5 or 6 membered heteroaryl can be typically substituted with 1-3 substituents independently selected from F, Cl, Br, CN, C₁₋₄ alkyl optionally substituted with 1-3 F, OH, cyclopropyl, cyclobutyl, or C₁₋₄ alkoxy optionally substituted with 1-3 F. In some embodiments, when substituted, the 5 or 6 membered heteroaryl can be substituted with 1-3 substituents (preferably 1) independently selected from (1) F, Cl, Br, OH, or CN, (2) C₁₋₄ alkyl optionally substituted with 1-3 F, (3) hydroxyl substituted C₁₋₄ alkyl, (4) cyclopropyl or cyclobutyl, each optionally substituted 1 or 2 substituents independently F, methyl, CN, or OH, (5) C₂₋₄ alkynyl optionally substituted with 1-3 F; or (6) C₁₋₄ heteroalkyl having 1 or 2 heteroatoms independently selected from O and N, which is optionally substituted with 1-3 F. In some embodiments, when substituted, the 5 or 6 membered heteroaryl can be substituted with 1 or 2 substituents, preferably one substituent, independently selected from F, Cl, CN, C₁₋₄ alkyl optionally substituted with 1-3 F (e.g., CHF₂ or CF₃), or cyclopropyl.

In some specific embodiments, Ring B in Formula I (e.g., any of the applicable sub-formulae as described herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c) can be

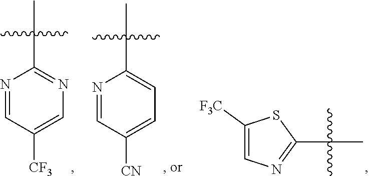

or ring B is

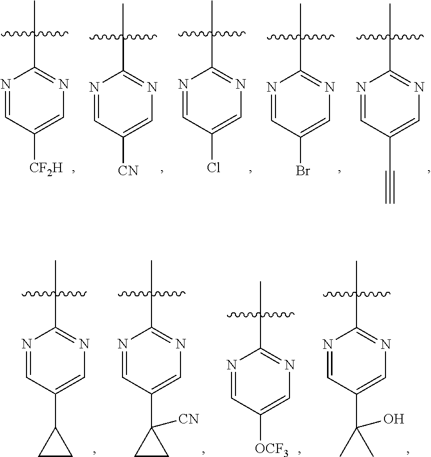

or ring B is

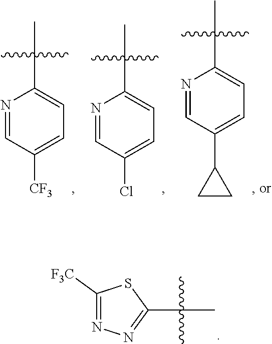

In some embodiments according to Formula I, when L³ is null, ring A and ring B can together represent an optionally substituted cyclic structure having one ring or at least two rings, e.g., a bicyclic structure. For example, in some embodiments, ring A and ring B together is an optionally substituted monocyclic aromatic or heteroaromatic ring, in other words, one of ring A and ring B does not exist. In some embodiments, ring A and ring B together is an optionally substituted cyclic structure having at least two rings, e.g., bicyclic ring, such as a bicyclic heteroaryl or heterocyclic ring. In some embodiments, L³ is null, and as applicable, ring A and ring B together represent an optionally substituted cyclic structure, such as an optionally substituted piperidine, piperazine, or a fused tetrahydro triazolopyrimidine ring, e.g.,

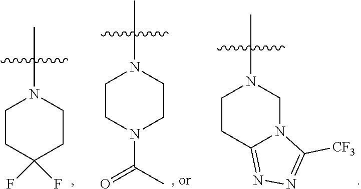

The combinations of the variables in the various formulae herein are not particularly limited, which include any of those applicable combinations exemplified with the specific compounds shown herein such as those shown in the Examples section or Table A herein.

In some embodiments, -X-(Ring A)-L³-(Ring B) in any of the applicable formulae herein, such as I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, can be

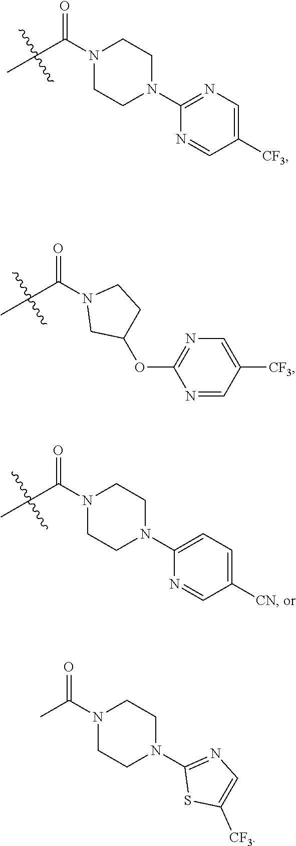

In some preferred embodiments, -X-(Ring A)-L³-(Ring B) in any of the applicable formulae herein can be

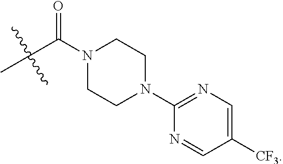 [SMILES: *C(=O)N1CCN(c2ncc(C(F)(F)F)cn2)CC1]

In some specific embodiments, -X-(Ring A)-L³-(Ring B) in any of the applicable formulae herein can be characterized as having the structure of

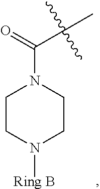

wherein Ring B is

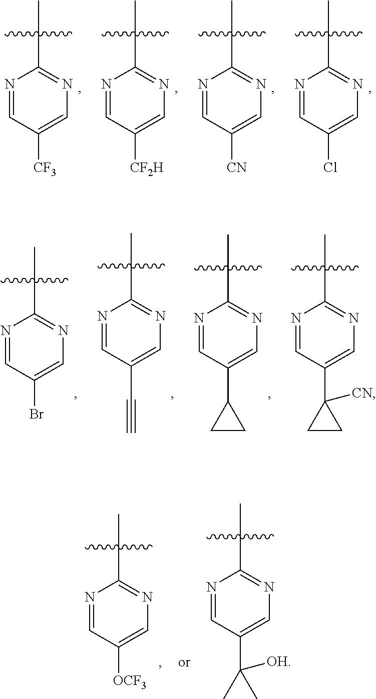

In some specific embodiments, -X-(Ring A)-L³-(Ring B) in any of the applicable formulae herein can be characterized as having the structure of

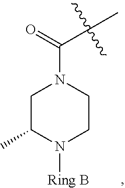

wherein Ring B is

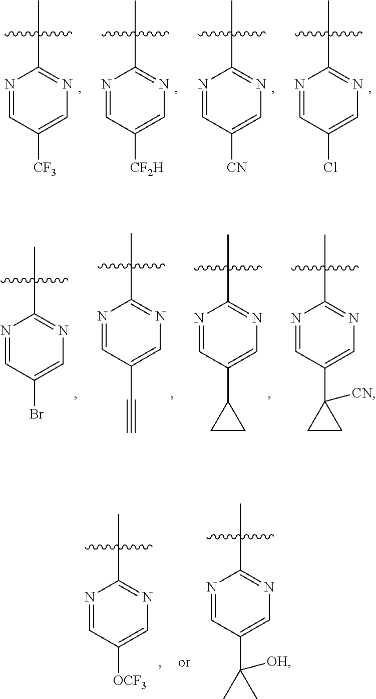

for example, -X-(Ring A)-L³-(Ring B) is

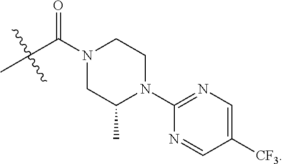 [SMILES: *C(=O)N1CCN(c2ncc(C(F)(F)F)cn2)[C@H](C)C1]

In some specific embodiments, -X-(Ring A)-L³-(Ring B) in any of the applicable formulae herein can be characterized as having the structure of

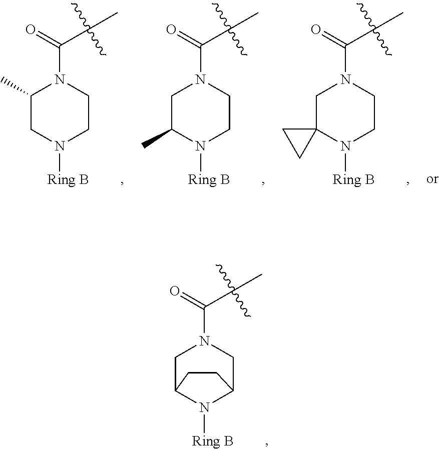

wherein Ring B is

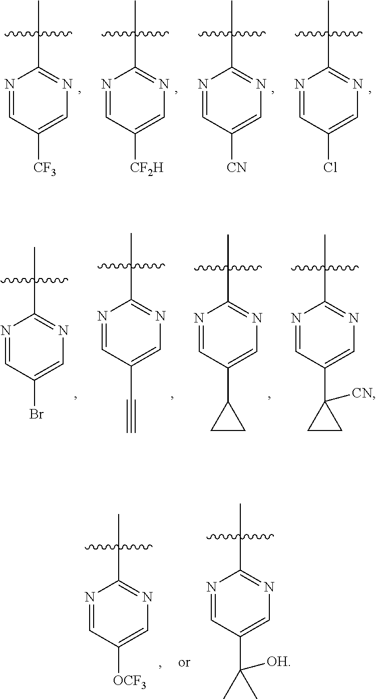

In some specific embodiments, -X-(Ring A)-L³-(Ring B) in any of the applicable formulae herein can be characterized as having the structure of

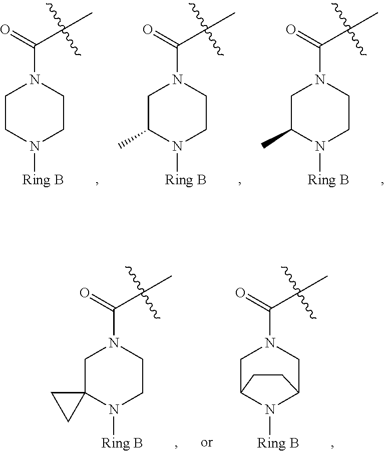

wherein Ring B is

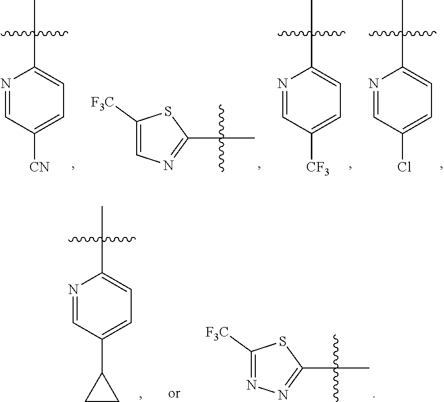

The present disclosure also provides the following exemplified embodiments according to the subformulae of Formula I as described herein:

Embodiment 1. A compound of Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, wherein the variables of the respective formula are defined herein, or a pharmaceutically acceptable salt thereof.

Embodiment 2. The compound of Embodiment 1, or a pharmaceutically acceptable salt thereof, wherein R¹ in Formula I-1, I-B, I-B-1, I-B-2, I-B-3, I-B-1-E1, I-B-1-E2, I-B-2-E1, I-B-2-E2, I-B-1-E1-a, I-B-1-E2-a, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c is hydrogen, CH₃, ethyl, isopropyl, cyclopropyl, CN, OCH₃, SCH₃, CF₃, F, Cl, Br, CF₂H,

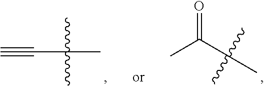

or R¹ is OCH₂CF₂H.

Embodiment 3. The compound of Embodiment 1 or 2, or a pharmaceutically acceptable salt thereof, wherein R² in Formula I-1, I-2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c is hydrogen, CH₃, CF₃,

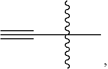 [SMILES: *C#C]

NH₂, NHCH₃,

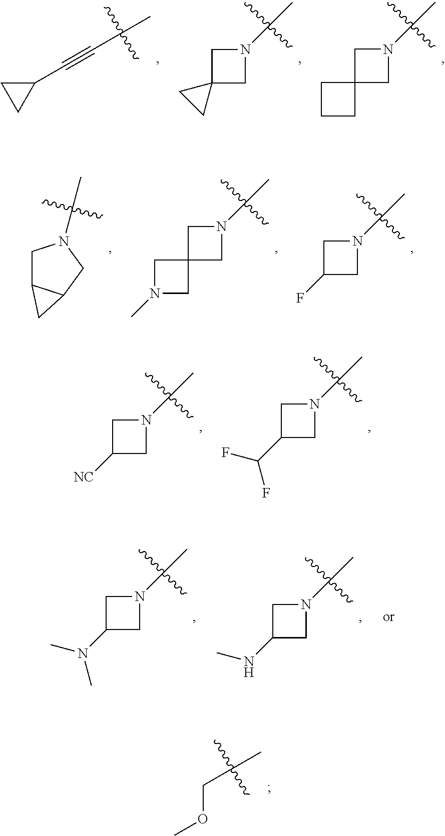

### or R² is

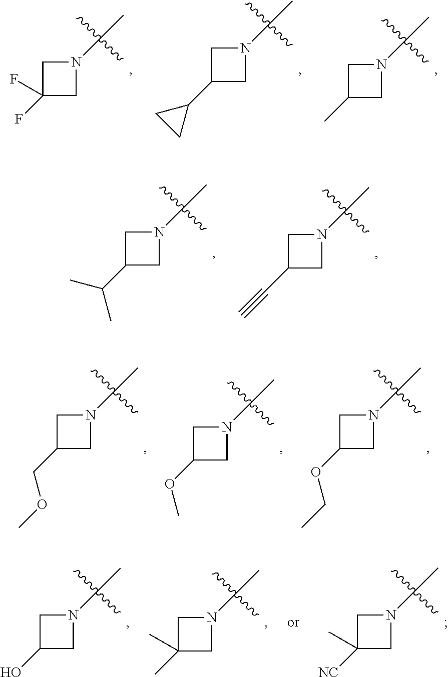

### or R² is

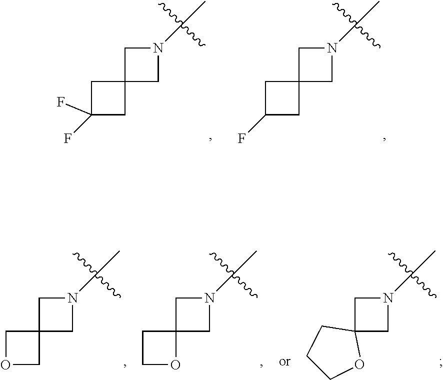 [SMILES: *N1CC2(C1)CC(F)(F)C2 ; *N1CC2(CC(F)C2)C1 ; *N1CC2(COC2)C1 ; *N1CC2(CCO2)C1 ; *N1CC2(CCCO2)C1]

### or R² is

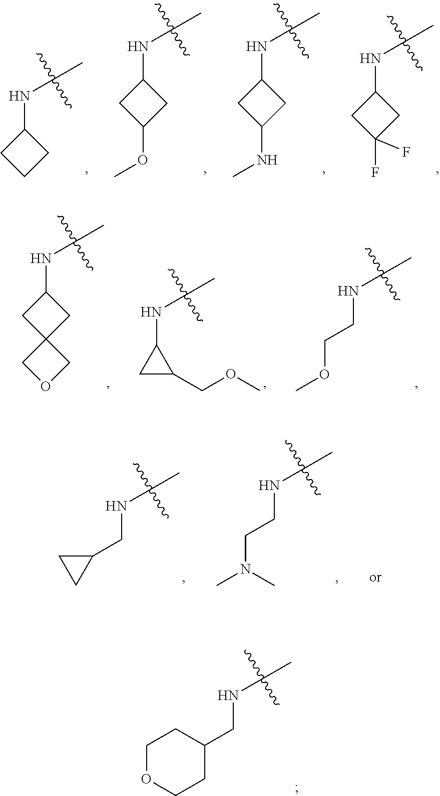

### or R² is

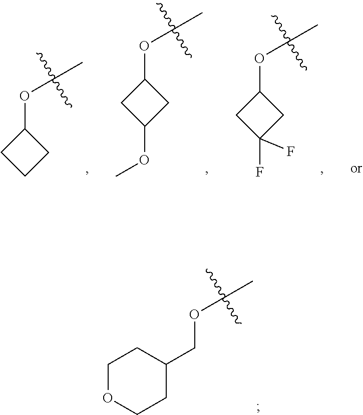

or R² is cyclopropyl.

Embodiment 4. The compound of Embodiment 1, or a pharmaceutically acceptable salt thereof, wherein in Formula I-1, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, R¹ and R², together with the intervening atoms, are joined to form a ring structure selected from

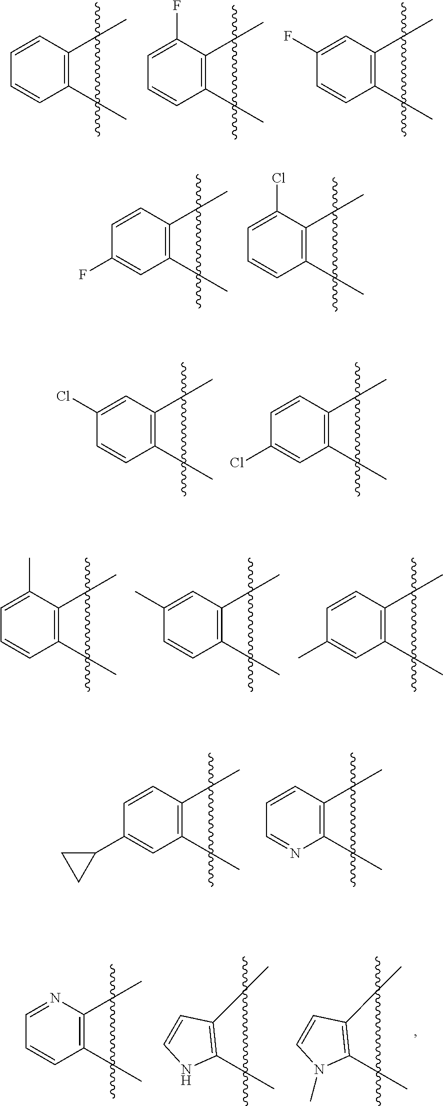

or selected from:

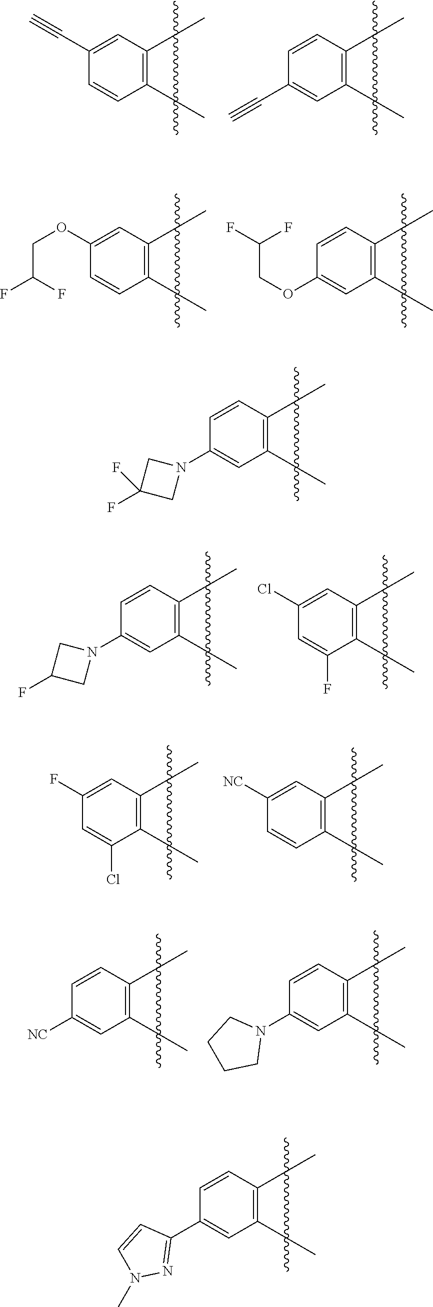

wherein the top connecting point of the fragments above is to the carbonyl group in the respective formulae.

Embodiment 5. The compound of any of Embodiments 1-4, or a pharmaceutically acceptable salt thereof, wherein in Formula I-1, I-2, I-A, I-A-1, I-A-2, or I-A-3, L¹ is an optionally substituted ring selected from:

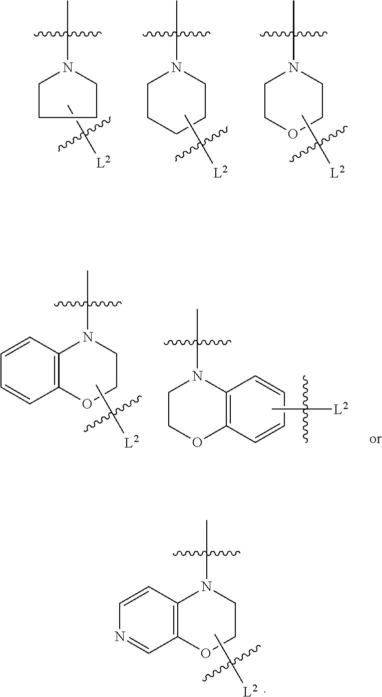

Embodiment 6. The compound of Embodiment 5, or a pharmaceutically acceptable salt thereof, wherein the optionally substituted ring, when substituted, is substituted with one or more (e.g., 1-5 or 1-3) substituents, each independently halo (preferably F or C₁), CN, C₁₋₄ alkyl optionally substituted with 1-5 (e.g., 1, 2, or 3) GP, or cyclopropyl optionally substituted with 1-5 (e.g., 1, 2, or 3) GP, wherein GP at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F.

Embodiment 7. The compound of Embodiment 5, or a pharmaceutically acceptable salt thereof, wherein the optionally substituted ring, when substituted, is substituted with one or two substituents each independently F or methyl, or in Formula I-C-1-a, g1 is 1, and R^{G} is F, C₁, CN, cyclopropyl, or C₁₋₄ alkyl, preferably, R^{G} is at a position para to the oxygen atom; or in Formula I-C-1-al or I-C-1-a2, R^{G} is F, Cl, CN, C₁₋₄ alkyl optionally substituted with 1-3 F, cyclopropyl,

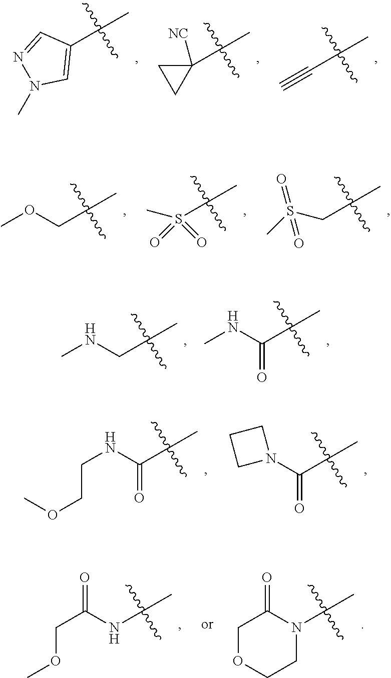

Embodiment 8. The compound of Embodiment 5, or a pharmaceutically acceptable salt thereof, wherein the optionally substituted ring is selected from the following:

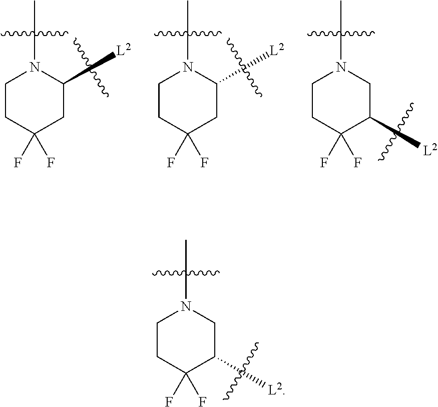

Embodiment 8. The compound of Embodiment 5, or a pharmaceutically acceptable salt thereof, wherein the optionally substituted ring is selected from the following:

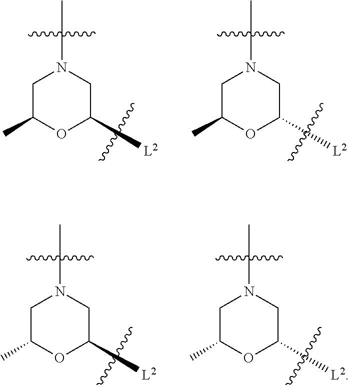

Embodiment 9. The compound of any of Embodiments 1-4, or a pharmaceutically acceptable salt thereof, wherein in Formula I-1, I-2, I-A, I-A-1, I-A-2, I-A-3, I-D-1, I-D-2, I-D-3, I-D-1-a, I-D-2-a, or I-D-3-a, L¹ is O, S, NH, or NCH₃.

Embodiment 10. The compound of any of Embodiments 1-2, or a pharmaceutically acceptable salt thereof, wherein in Formula I-1, I-2, I-D-1, I-D-2, I-D-3, I-D-1-a, I-D-2-a, or I-D-3-a, R² and L¹, together with the intervening atoms, are joined to form an optionally substituted 5-7 membered heterocyclyl having one ring heteroatom, which is O or N, wherein suitable substituents are described herein.

Embodiment 11. The compound of Embodiment 10, or a pharmaceutically acceptable salt thereof, wherein the optionally substituted 5-7 membered heterocyclyl having one ring heteroatom, when substituted, is substituted with 1-3 (e.g., 1, 2, or 3) substituents independently selected from methyl, phenyl,

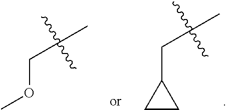

Embodiment 12. The compound of any of Embodiments 1-11, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4, L² is null (i.e., X is directly attached to L¹).

Embodiment 13. The compound of any of Embodiments 1-11, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4, L² is a phenylene selected from:

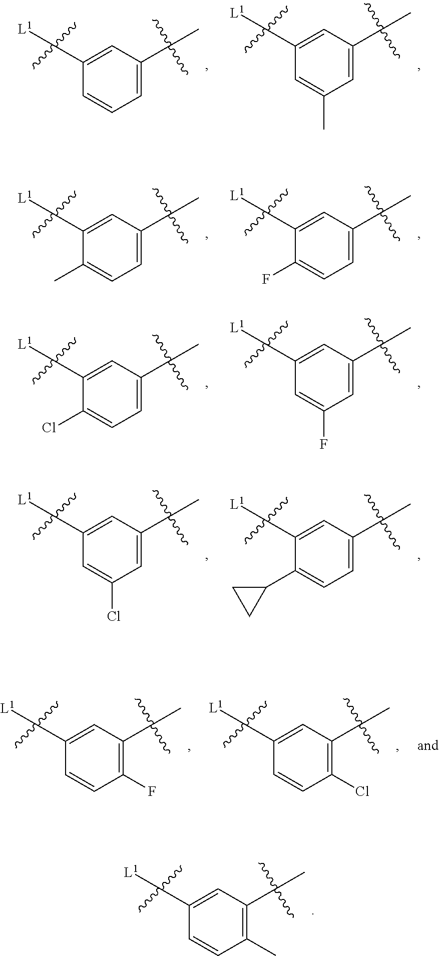

Embodiment 14. The compound of any of Embodiments 1-11, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4, L² is

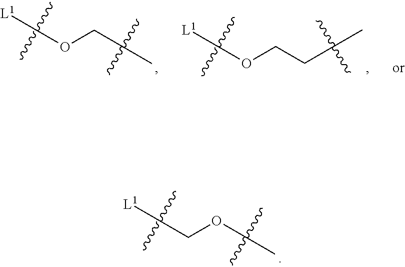

Embodiment 15. The compound of any of Embodiments 1-11, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4, L² is

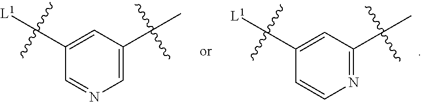

Embodiment 16. The compound of any of Embodiments 1-11, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, or I-C-3-E4, L² is C₁₋₄ alkylene, preferably, methylene, or ethylene.

Embodiment 17. The compound of any of Embodiments 1-16, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, X is C(O).

Embodiment 18. The compound of any of Embodiments 1-17, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, X is

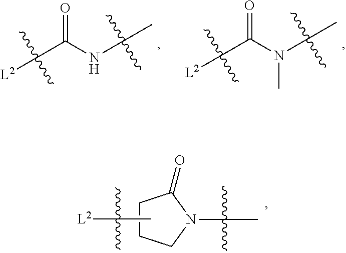

### or S(O)₂.

Embodiment 19. The compound of any of Embodiments 1-18, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, Ring

A is

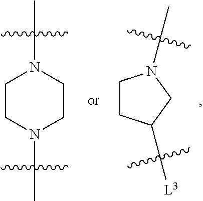

which is optionally substituted as described herein, for example, Ring A is

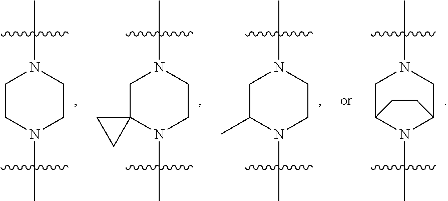

Embodiment 20. The compound of any of Embodiments 1-19, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, L³ is null.

Embodiment 21. The compound of any of Embodiments 1-19, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, L³ is O, NH, or NCH₃.

Embodiment 22. The compound of any of Embodiments 1-21, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, Ring B is an optionally substituted 5 or 6 membered heteroaryl selected from pyridine, pyrazine, thiazole, thiadiazole, and pyrimidine, suitable substituents are described herein.

Embodiment 23. The compound of any of Embodiments 1-21, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, Ring B is

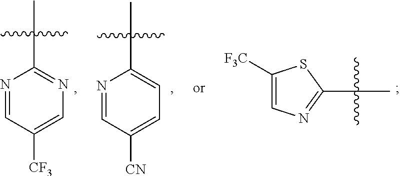

or ring B is

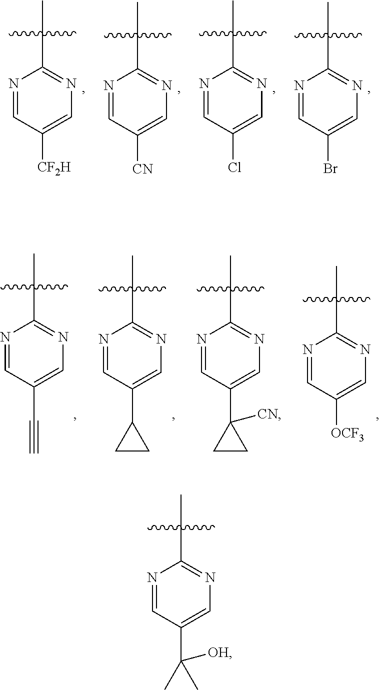

or ring B is

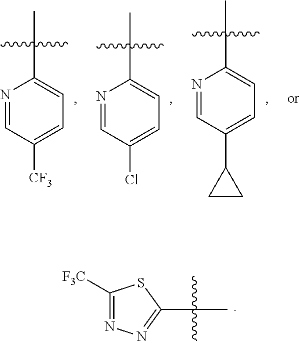

Embodiment 24. The compound of any of Embodiments 1-16, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, -X-(Ring A)-L³-(Ring B) is

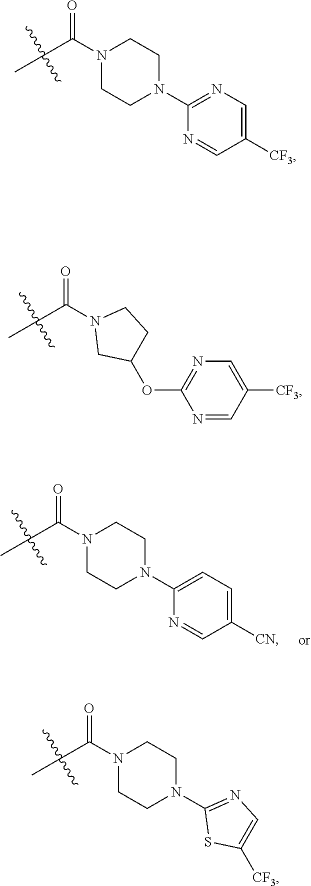 [SMILES: *C(=O)N1CCN(c2ncc(C(F)(F)F)cn2)CC1 ; *C(=O)N1CCC(Oc2ncc(C(F)(F)F)cn2)C1 ; *C(=O)N1CCN(c2ccc(C#N)cn2)CC1 ; *C(=O)N1CCN(c2ncc(C(F)(F)F)s2)CC1]

or any of those exemplified in the Examples or the compounds in Table A.

Embodiment 25. The compound of any of Embodiments 1-16, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, -X-(Ring A)-L³-(Ring B) can be characterized as having the structure of

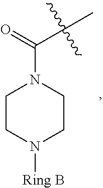

wherein Ring B is

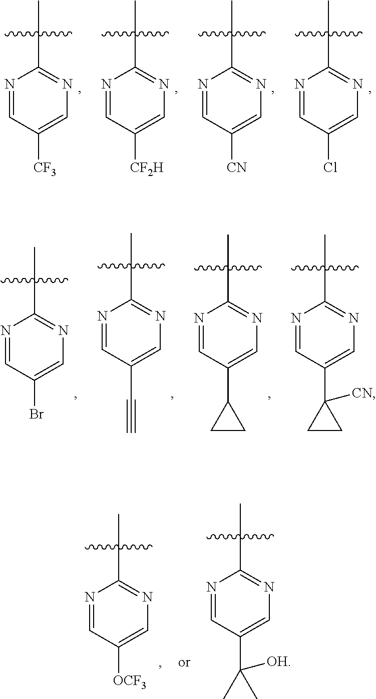

Embodiment 26. The compound of any of Embodiments 1-16, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, -X-(Ring A)-L³-(Ring B) can be characterized as having the structure of

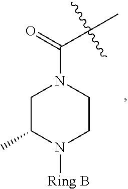

wherein Ring B is

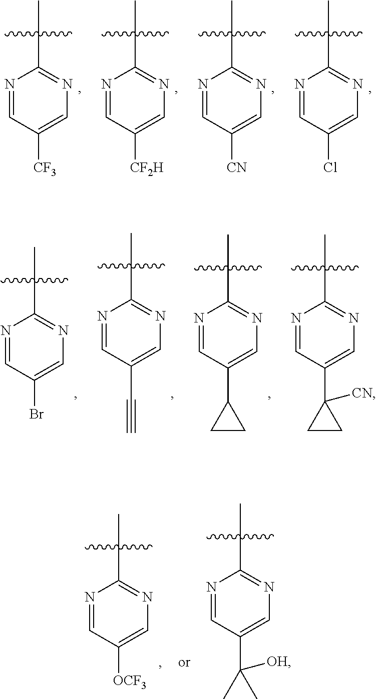

for example, -X-(Ring A)-L³-(Ring B) is

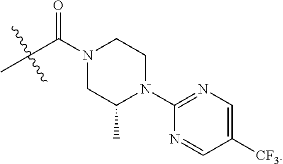 [SMILES: *C(=O)N1CCN(c2ncc(C(F)(F)F)cn2)[C@H](C)C1]

Embodiment 27. The compound of any of Embodiments 1-16, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, -X-(Ring A)-L³-(Ring B) can be characterized as having the structure of

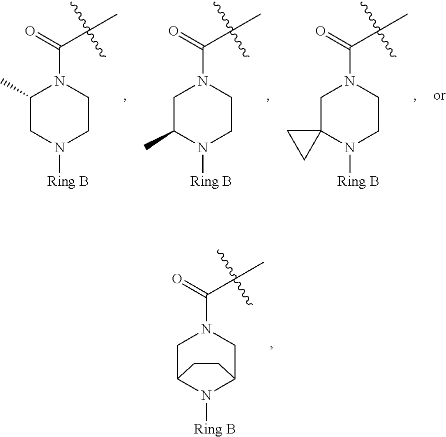

wherein Ring B is

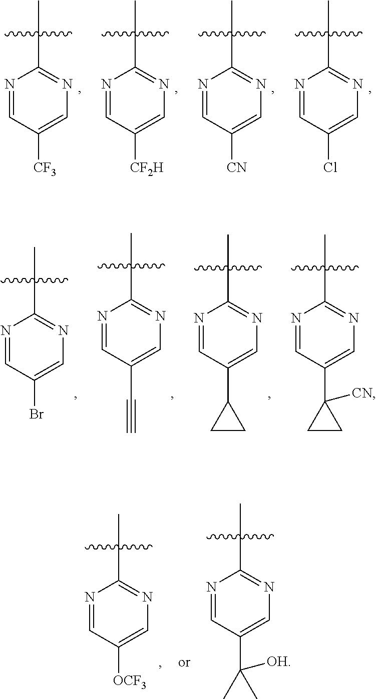

Embodiment 28. The compound of any of Embodiments 1-16, or a pharmaceutically acceptable salt thereof, wherein in Formula I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-a1, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c, -X-(Ring A)-L³-(Ring B) can be characterized as having the structure of

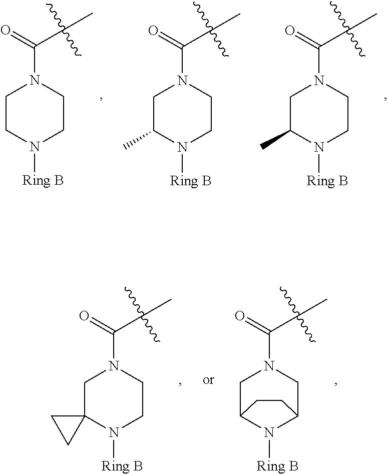

wherein Ring B is

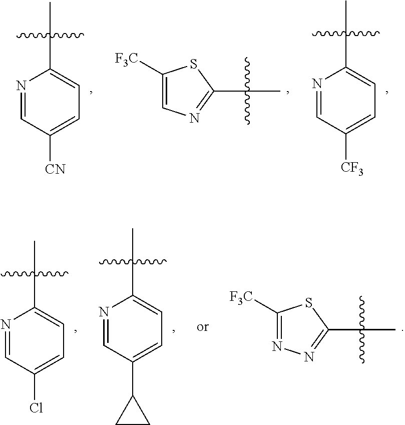

In some embodiments, the present disclosure also provides a compound selected from Table A below, a deuterated analog thereof, a stereoisomer thereof, or a pharmaceutically acceptable salt thereof:

TABLE A
List of Compounds

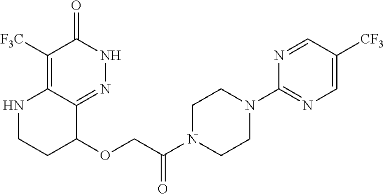 [SMILES: O=C(COC1CCNc2c1n[nH]c(=O)c2C(F)(F)F)N1CCN(c2ncc(C(F)(F)F)cn2)CC1]

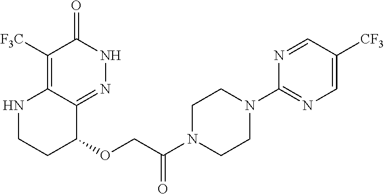 [SMILES: O=C(CO[C@@H]1CCNc2c1n[nH]c(=O)c2C(F)(F)F)N1CCN(c2ncc(C(F)(F)F)cn2)CC1]

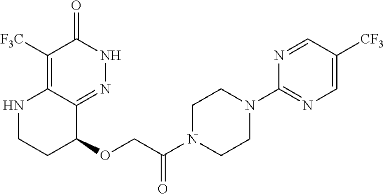 [SMILES: O=C(CO[C@H]1CCNc2c1n[nH]c(=O)c2C(F)(F)F)N1CCN(c2ncc(C(F)(F)F)cn2)CC1]

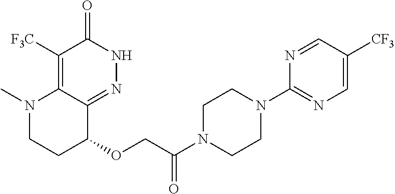 [SMILES: CN1CC[C@@H](OCC(=O)N2CCN(c3ncc(C(F)(F)F)cn3)CC2)c2n[nH]c(=O)c(C(F)(F)F)c21]

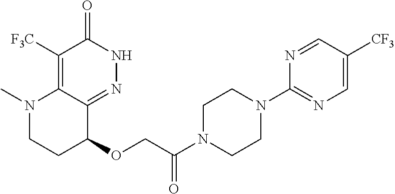 [SMILES: CN1CC[C@H](OCC(=O)N2CCN(c3ncc(C(F)(F)F)cn3)CC2)c2n[nH]c(=O)c(C(F)(F)F)c21]

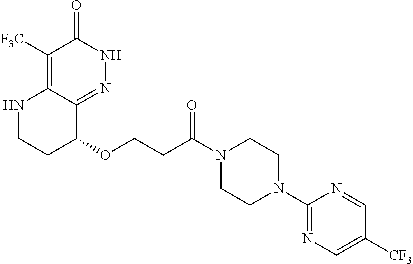 [SMILES: O=C(CCO[C@@H]1CCNc2c1n[nH]c(=O)c2C(F)(F)F)N1CCN(c2ncc(C(F)(F)F)cn2)CC1]

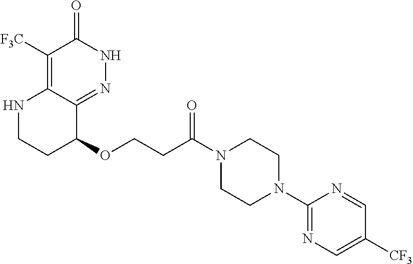 [SMILES: O=C(CCO[C@H]1CCNc2c1n[nH]c(=O)c2C(F)(F)F)N1CCN(c2ncc(C(F)(F)F)cn2)CC1]

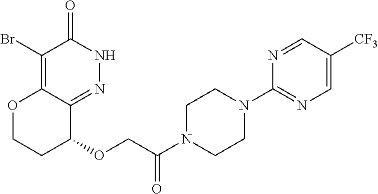 [SMILES: O=C(CO[C@@H]1CCOc2c1n[nH]c(=O)c2Br)N1CCN(c2ncc(C(F)(F)F)cn2)CC1]

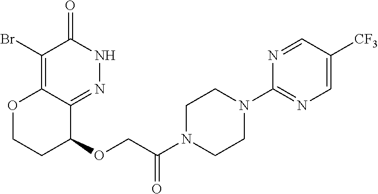 [SMILES: O=C(CO[C@H]1CCOc2c1n[nH]c(=O)c2Br)N1CCN(c2ncc(C(F)(F)F)cn2)CC1]

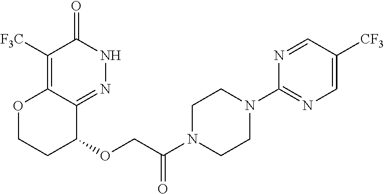 [SMILES: O=C(CO[C@@H]1CCOc2c1n[nH]c(=O)c2C(F)(F)F)N1CCN(c2ncc(C(F)(F)F)cn2)CC1]

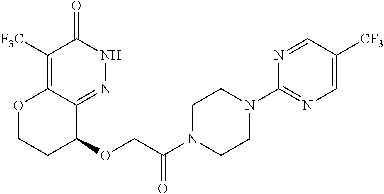 [SMILES: O=C(CO[C@H]1CCOc2c1n[nH]c(=O)c2C(F)(F)F)N1CCN(c2ncc(C(F)(F)F)cn2)CC1]

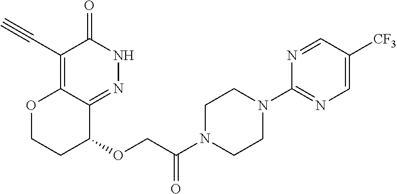 [SMILES: C#Cc1c2c(n[nH]c1=O)[C@H](OCC(=O)N1CCN(c3ncc(C(F)(F)F)cn3)CC1)CCO2]

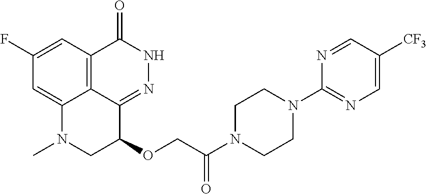 [SMILES: CN1C[C@H](OCC(=O)N2CCN(c3ncc(C(F)(F)F)cn3)CC2)c2n[nH]c(=O)c3cc(F)cc1c23]

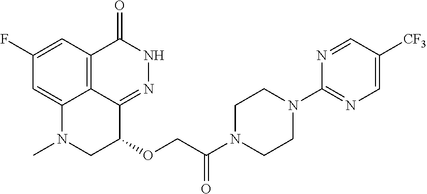 [SMILES: CN1C[C@@H](OCC(=O)N2CCN(c3ncc(C(F)(F)F)cn3)CC2)c2n[nH]c(=O)c3cc(F)cc1c23]

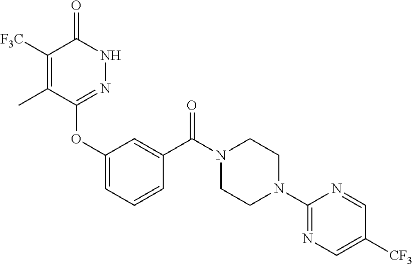 [SMILES: Cc1c(Oc2cccc(C(=O)N3CCN(c4ncc(C(F)(F)F)cn4)CC3)c2)n[nH]c(=O)c1C(F)(F)F]

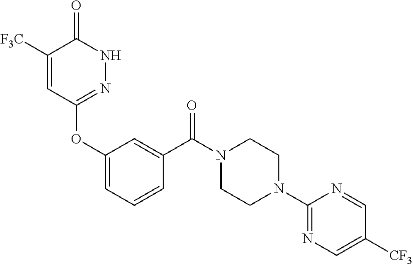 [SMILES: O=C(c1cccc(Oc2cc(C(F)(F)F)c(=O)[nH]n2)c1)N1CCN(c2ncc(C(F)(F)F)cn2)CC1]

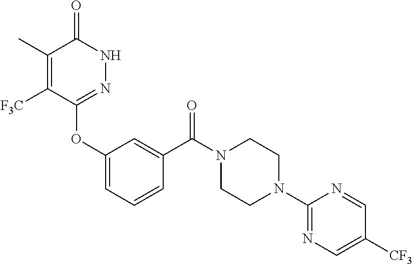 [SMILES: Cc1c(C(F)(F)F)c(Oc2cccc(C(=O)N3CCN(c4ncc(C(F)(F)F)cn4)CC3)c2)n[nH]c1=O]

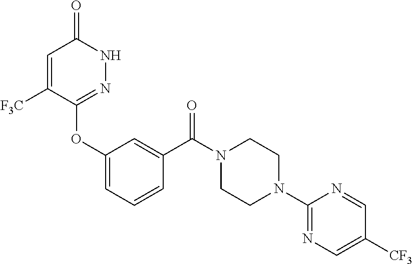 [SMILES: O=C(c1cccc(Oc2n[nH]c(=O)cc2C(F)(F)F)c1)N1CCN(c2ncc(C(F)(F)F)cn2)CC1]

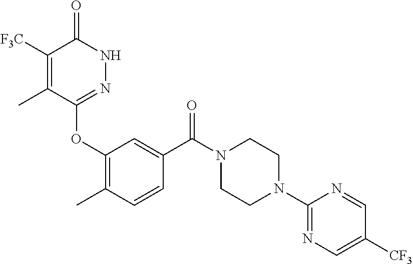 [SMILES: Cc1ccc(C(=O)N2CCN(c3ncc(C(F)(F)F)cn3)CC2)cc1Oc1n[nH]c(=O)c(C(F)(F)F)c1C]

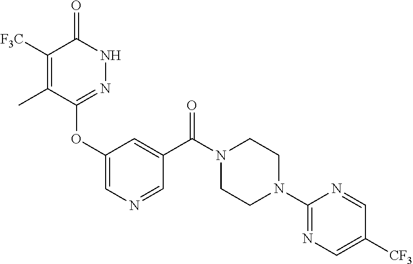 [SMILES: Cc1c(Oc2cncc(C(=O)N3CCN(c4ncc(C(F)(F)F)cn4)CC3)c2)n[nH]c(=O)c1C(F)(F)F]

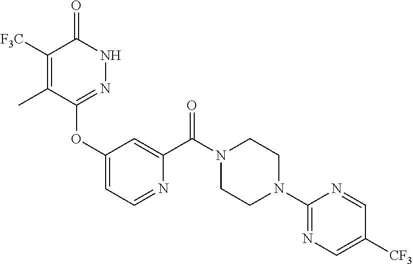 [SMILES: Cc1c(Oc2ccnc(C(=O)N3CCN(c4ncc(C(F)(F)F)cn4)CC3)c2)n[nH]c(=O)c1C(F)(F)F]

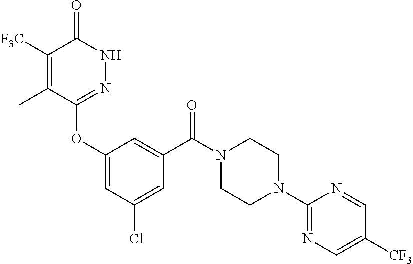 [SMILES: Cc1c(Oc2cc(Cl)cc(C(=O)N3CCN(c4ncc(C(F)(F)F)cn4)CC3)c2)n[nH]c(=O)c1C(F)(F)F]

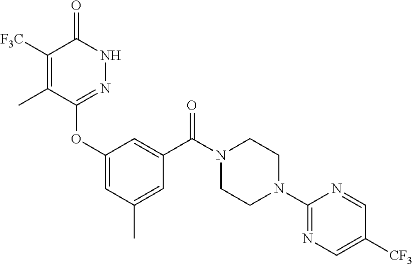 [SMILES: Cc1cc(Oc2n[nH]c(=O)c(C(F)(F)F)c2C)cc(C(=O)N2CCN(c3ncc(C(F)(F)F)cn3)CC2)c1]

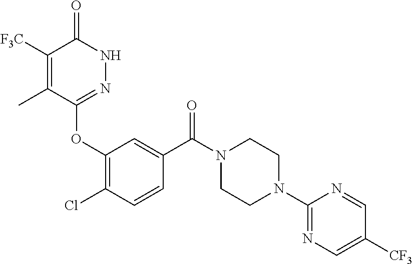 [SMILES: Cc1c(Oc2cc(C(=O)N3CCN(c4ncc(C(F)(F)F)cn4)CC3)ccc2Cl)n[nH]c(=O)c1C(F)(F)F]

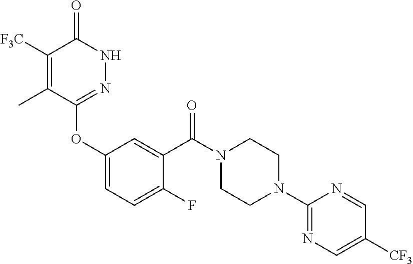 [SMILES: Cc1c(Oc2ccc(F)c(C(=O)N3CCN(c4ncc(C(F)(F)F)cn4)CC3)c2)n[nH]c(=O)c1C(F)(F)F]

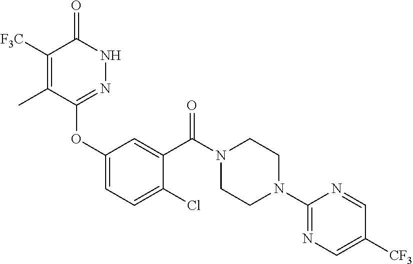 [SMILES: Cc1c(Oc2ccc(Cl)c(C(=O)N3CCN(c4ncc(C(F)(F)F)cn4)CC3)c2)n[nH]c(=O)c1C(F)(F)F]

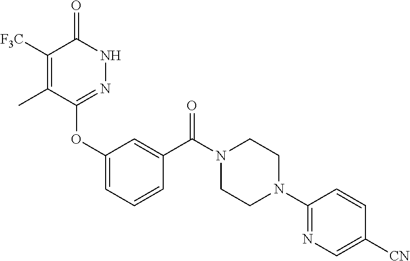 [SMILES: Cc1c(Oc2cccc(C(=O)N3CCN(c4ccc(C#N)cn4)CC3)c2)n[nH]c(=O)c1C(F)(F)F]

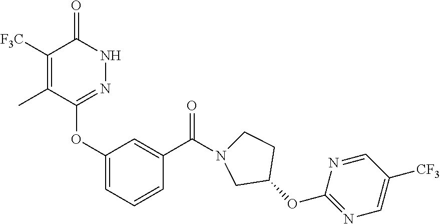 [SMILES: Cc1c(Oc2cccc(C(=O)N3CC[C@H](Oc4ncc(C(F)(F)F)cn4)C3)c2)n[nH]c(=O)c1C(F)(F)F]

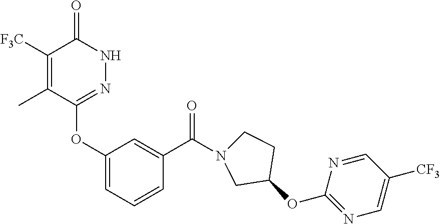 [SMILES: Cc1c(Oc2cccc(C(=O)N3CC[C@@H](Oc4ncc(C(F)(F)F)cn4)C3)c2)n[nH]c(=O)c1C(F)(F)F]

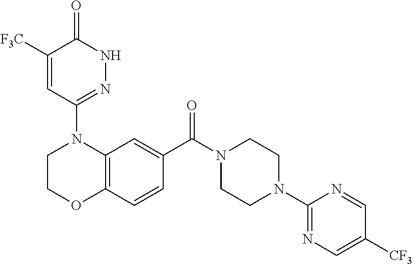 [SMILES: O=C(c1ccc2c(c1)N(c1cc(C(F)(F)F)c(=O)[nH]n1)CCO2)N1CCN(c2ncc(C(F)(F)F)cn2)CC1]

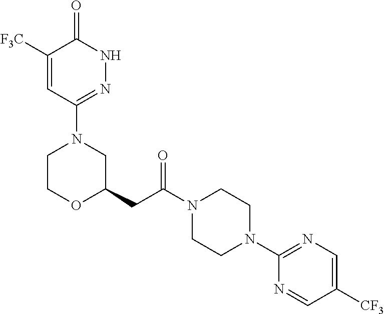 [SMILES: O=C(C[C@@H]1CN(c2cc(C(F)(F)F)c(=O)[nH]n2)CCO1)N1CCN(c2ncc(C(F)(F)F)cn2)CC1]

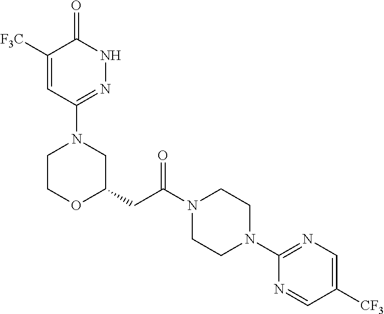 [SMILES: O=C(C[C@H]1CN(c2cc(C(F)(F)F)c(=O)[nH]n2)CCO1)N1CCN(c2ncc(C(F)(F)F)cn2)CC1]

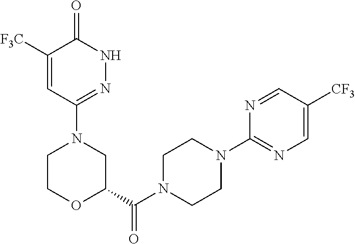 [SMILES: O=C([C@H]1CN(c2cc(C(F)(F)F)c(=O)[nH]n2)CCO1)N1CCN(c2ncc(C(F)(F)F)cn2)CC1]

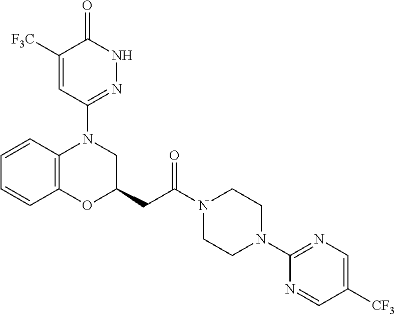 [SMILES: O=C(C[C@@H]1CN(c2cc(C(F)(F)F)c(=O)[nH]n2)c2ccccc2O1)N1CCN(c2ncc(C(F)(F)F)cn2)CC1]

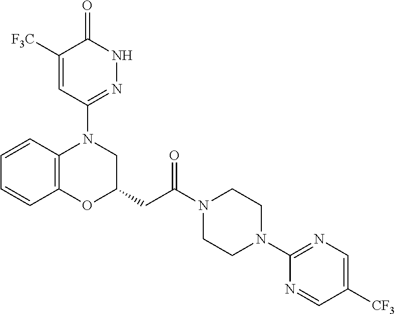 [SMILES: O=C(C[C@H]1CN(c2cc(C(F)(F)F)c(=O)[nH]n2)c2ccccc2O1)N1CCN(c2ncc(C(F)(F)F)cn2)CC1]

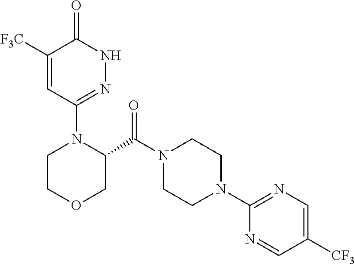 [SMILES: O=C([C@@H]1COCCN1c1cc(C(F)(F)F)c(=O)[nH]n1)N1CCN(c2ncc(C(F)(F)F)cn2)CC1]

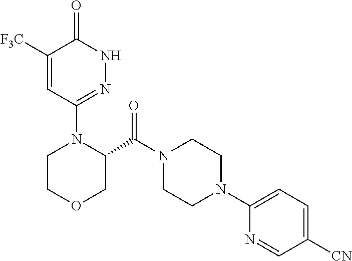 [SMILES: N#Cc1ccc(N2CCN(C(=O)[C@@H]3COCCN3c3cc(C(F)(F)F)c(=O)[nH]n3)CC2)nc1]

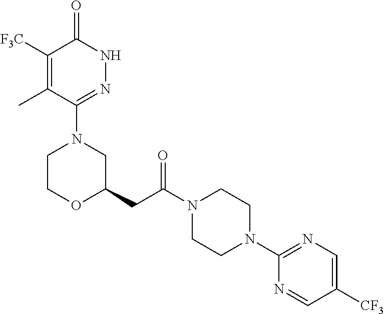 [SMILES: Cc1c(N2CCO[C@H](CC(=O)N3CCN(c4ncc(C(F)(F)F)cn4)CC3)C2)n[nH]c(=O)c1C(F)(F)F]

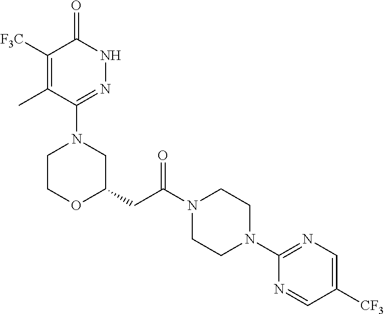 [SMILES: Cc1c(N2CCO[C@@H](CC(=O)N3CCN(c4ncc(C(F)(F)F)cn4)CC3)C2)n[nH]c(=O)c1C(F)(F)F]

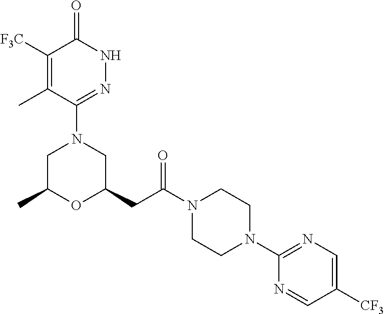 [SMILES: Cc1c(N2C[C@@H](CC(=O)N3CCN(c4ncc(C(F)(F)F)cn4)CC3)O[C@@H](C)C2)n[nH]c(=O)c1C(F)(F)F]

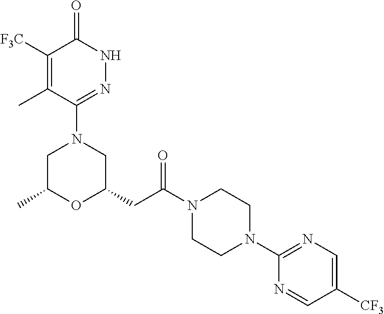 [SMILES: Cc1c(N2C[C@H](CC(=O)N3CCN(c4ncc(C(F)(F)F)cn4)CC3)O[C@H](C)C2)n[nH]c(=O)c1C(F)(F)F]

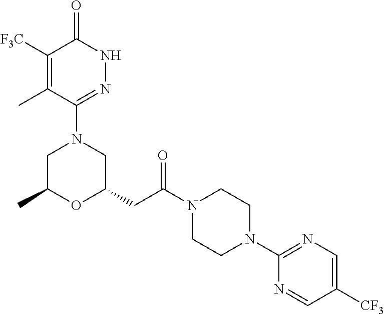 [SMILES: Cc1c(N2C[C@H](CC(=O)N3CCN(c4ncc(C(F)(F)F)cn4)CC3)O[C@@H](C)C2)n[nH]c(=O)c1C(F)(F)F]

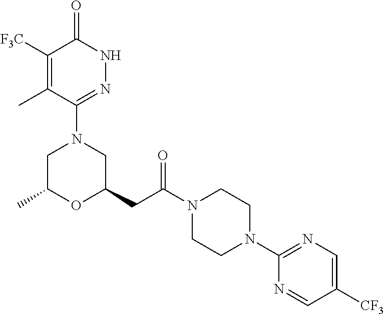 [SMILES: Cc1c(N2C[C@@H](CC(=O)N3CCN(c4ncc(C(F)(F)F)cn4)CC3)O[C@H](C)C2)n[nH]c(=O)c1C(F)(F)F]

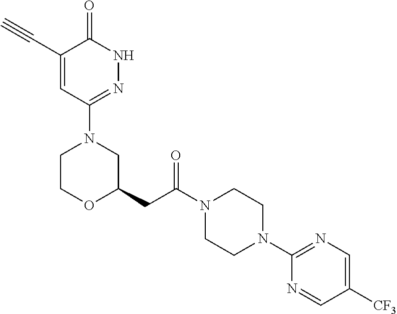 [SMILES: C#Cc1cc(N2CCO[C@H](CC(=O)N3CCN(c4ncc(C(F)(F)F)cn4)CC3)C2)n[nH]c1=O]

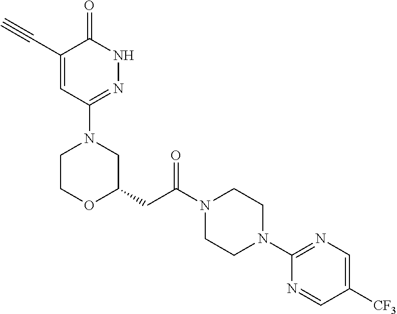 [SMILES: C#Cc1cc(N2CCO[C@@H](CC(=O)N3CCN(c4ncc(C(F)(F)F)cn4)CC3)C2)n[nH]c1=O]

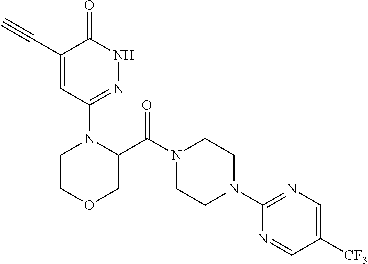 [SMILES: C#Cc1cc(N2CCOCC2C(=O)N2CCN(c3ncc(C(F)(F)F)cn3)CC2)n[nH]c1=O]

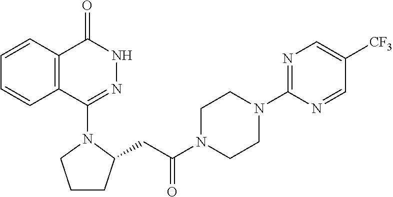 [SMILES: O=C(C[C@@H]1CCCN1c1n[nH]c(=O)c2ccccc12)N1CCN(c2ncc(C(F)(F)F)cn2)CC1]

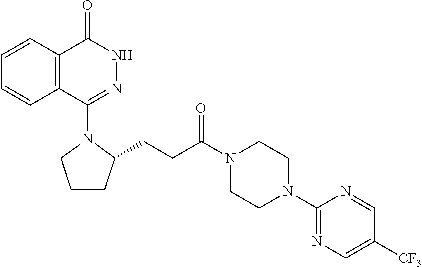 [SMILES: O=C(CC[C@@H]1CCCN1c1n[nH]c(=O)c2ccccc12)N1CCN(c2ncc(C(F)(F)F)cn2)CC1]

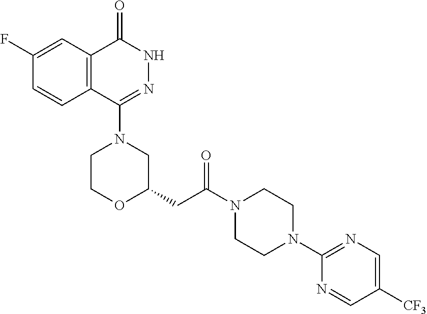 [SMILES: O=C(C[C@H]1CN(c2n[nH]c(=O)c3cc(F)ccc23)CCO1)N1CCN(c2ncc(C(F)(F)F)cn2)CC1]

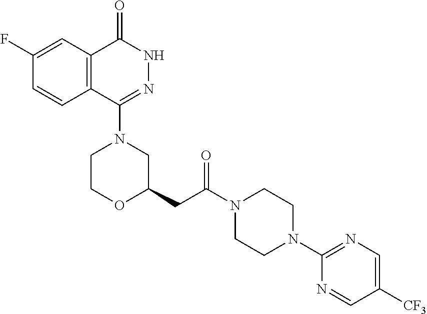 [SMILES: O=C(C[C@@H]1CN(c2n[nH]c(=O)c3cc(F)ccc23)CCO1)N1CCN(c2ncc(C(F)(F)F)cn2)CC1]

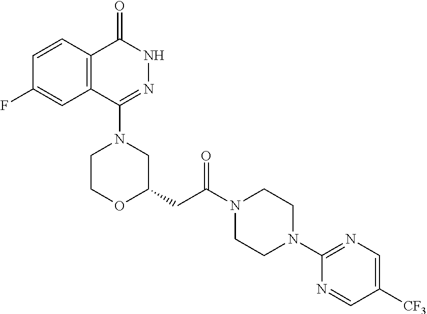 [SMILES: O=C(C[C@H]1CN(c2n[nH]c(=O)c3ccc(F)cc23)CCO1)N1CCN(c2ncc(C(F)(F)F)cn2)CC1]

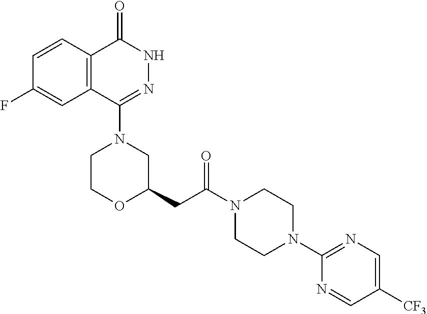 [SMILES: O=C(C[C@@H]1CN(c2n[nH]c(=O)c3ccc(F)cc23)CCO1)N1CCN(c2ncc(C(F)(F)F)cn2)CC1]

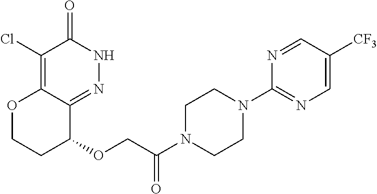 [SMILES: O=C(CO[C@@H]1CCOc2c1n[nH]c(=O)c2Cl)N1CCN(c2ncc(C(F)(F)F)cn2)CC1]

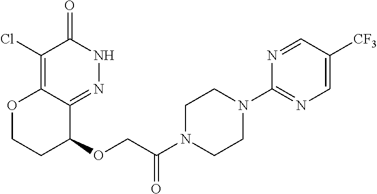 [SMILES: O=C(CO[C@H]1CCOc2c1n[nH]c(=O)c2Cl)N1CCN(c2ncc(C(F)(F)F)cn2)CC1]

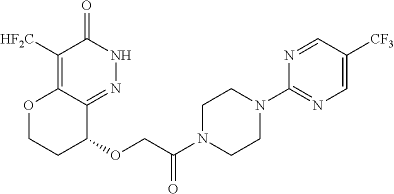

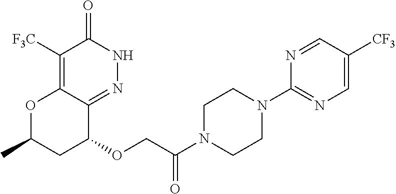 [SMILES: C[C@@H]1C[C@@H](OCC(=O)N2CCN(c3ncc(C(F)(F)F)cn3)CC2)c2n[nH]c(=O)c(C(F)(F)F)c2O1]

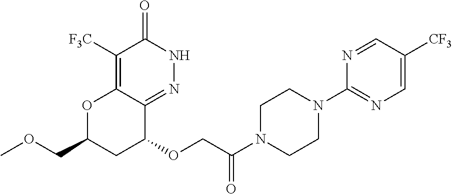 [SMILES: COC[C@@H]1C[C@@H](OCC(=O)N2CCN(c3ncc(C(F)(F)F)cn3)CC2)c2n[nH]c(=O)c(C(F)(F)F)c2O1]

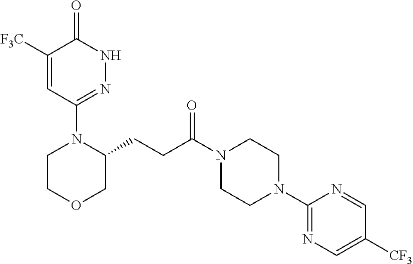 [SMILES: O=C(CC[C@@H]1COCCN1c1cc(C(F)(F)F)c(=O)[nH]n1)N1CCN(c2ncc(C(F)(F)F)cn2)CC1]

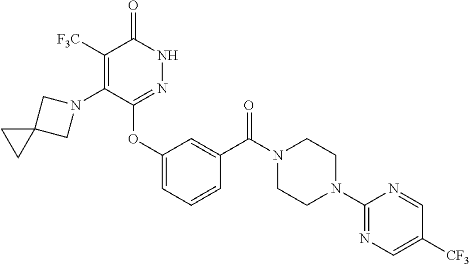 [SMILES: O=C(c1cccc(Oc2n[nH]c(=O)c(C(F)(F)F)c2N2CC3(CC3)C2)c1)N1CCN(c2ncc(C(F)(F)F)cn2)CC1]

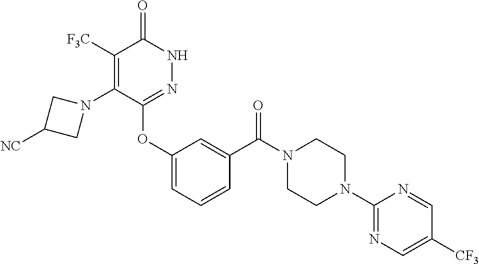 [SMILES: N#CC1CN(c2c(Oc3cccc(C(=O)N4CCN(c5ncc(C(F)(F)F)cn5)CC4)c3)n[nH]c(=O)c2C(F)(F)F)C1]

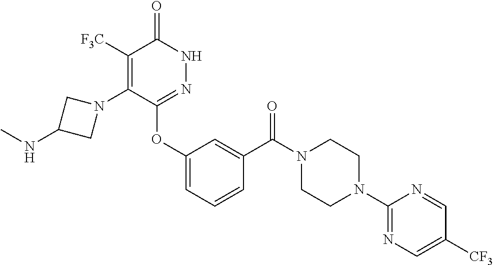 [SMILES: CNC1CN(c2c(Oc3cccc(C(=O)N4CCN(c5ncc(C(F)(F)F)cn5)CC4)c3)n[nH]c(=O)c2C(F)(F)F)C1]

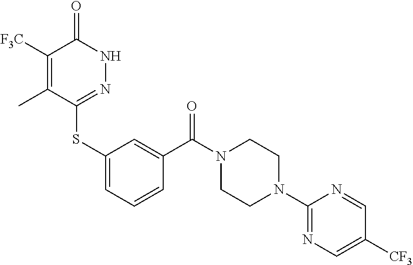 [SMILES: Cc1c(Sc2cccc(C(=O)N3CCN(c4ncc(C(F)(F)F)cn4)CC3)c2)n[nH]c(=O)c1C(F)(F)F]

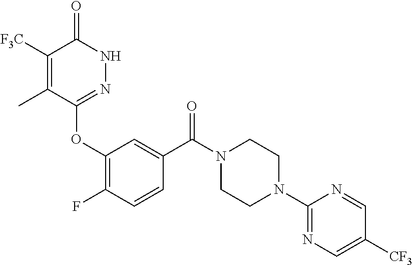 [SMILES: Cc1c(Oc2cc(C(=O)N3CCN(c4ncc(C(F)(F)F)cn4)CC3)ccc2F)n[nH]c(=O)c1C(F)(F)F]

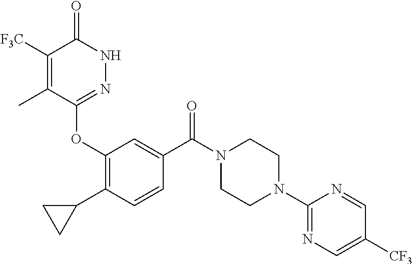 [SMILES: Cc1c(Oc2cc(C(=O)N3CCN(c4ncc(C(F)(F)F)cn4)CC3)ccc2C2CC2)n[nH]c(=O)c1C(F)(F)F]

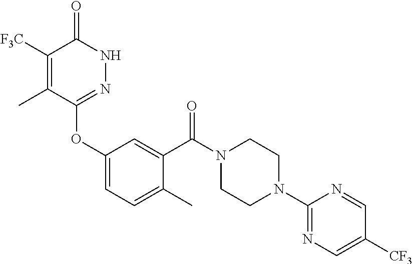 [SMILES: Cc1ccc(Oc2n[nH]c(=O)c(C(F)(F)F)c2C)cc1C(=O)N1CCN(c2ncc(C(F)(F)F)cn2)CC1]

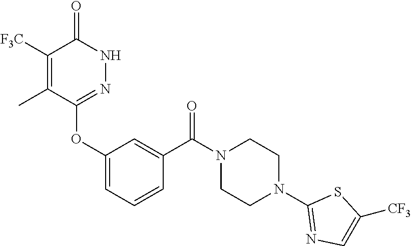 [SMILES: Cc1c(Oc2cccc(C(=O)N3CCN(c4ncc(C(F)(F)F)s4)CC3)c2)n[nH]c(=O)c1C(F)(F)F]

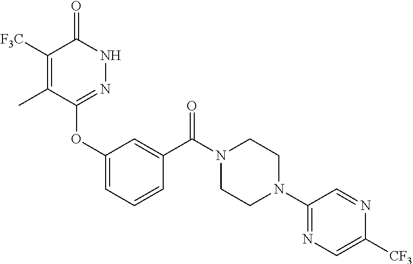 [SMILES: Cc1c(Oc2cccc(C(=O)N3CCN(c4cnc(C(F)(F)F)cn4)CC3)c2)n[nH]c(=O)c1C(F)(F)F]

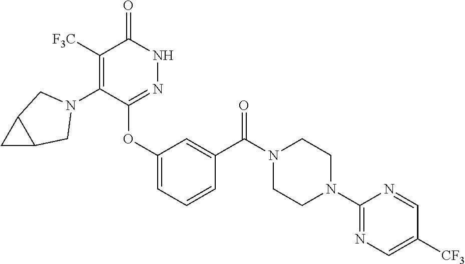 [SMILES: O=C(c1cccc(Oc2n[nH]c(=O)c(C(F)(F)F)c2N2CC3CC3C2)c1)N1CCN(c2ncc(C(F)(F)F)cn2)CC1]

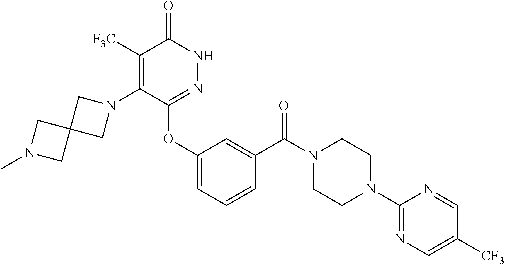 [SMILES: CN1CC2(C1)CN(c1c(Oc3cccc(C(=O)N4CCN(c5ncc(C(F)(F)F)cn5)CC4)c3)n[nH]c(=O)c1C(F)(F)F)C2]

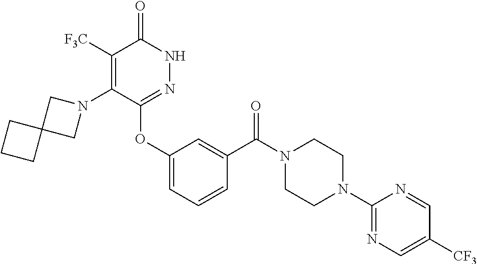 [SMILES: O=C(c1cccc(Oc2n[nH]c(=O)c(C(F)(F)F)c2N2CC3(CCC3)C2)c1)N1CCN(c2ncc(C(F)(F)F)cn2)CC1]

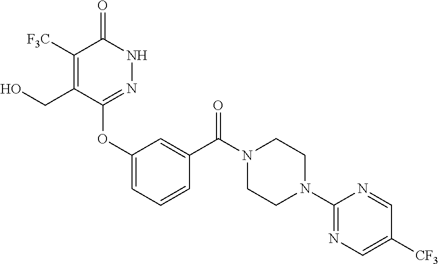 [SMILES: O=C(c1cccc(Oc2n[nH]c(=O)c(C(F)(F)F)c2CO)c1)N1CCN(c2ncc(C(F)(F)F)cn2)CC1]

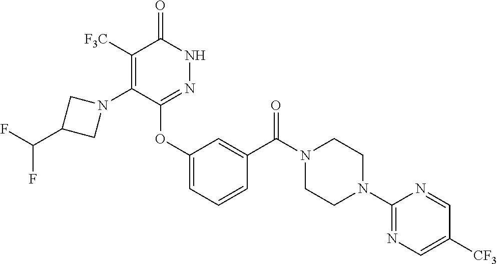 [SMILES: O=C(c1cccc(Oc2n[nH]c(=O)c(C(F)(F)F)c2N2CC(C(F)F)C2)c1)N1CCN(c2ncc(C(F)(F)F)cn2)CC1]

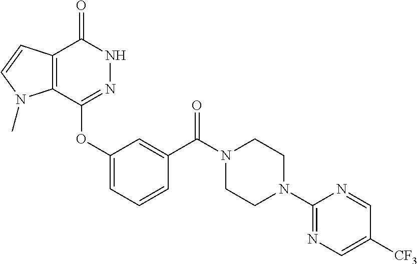 [SMILES: Cn1ccc2c(=O)[nH]nc(Oc3cccc(C(=O)N4CCN(c5ncc(C(F)(F)F)cn5)CC4)c3)c21]

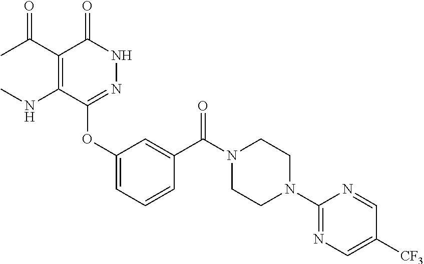 [SMILES: CNc1c(Oc2cccc(C(=O)N3CCN(c4ncc(C(F)(F)F)cn4)CC3)c2)n[nH]c(=O)c1C(C)=O]

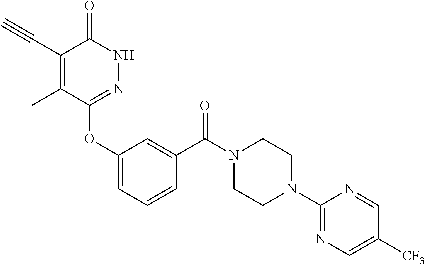 [SMILES: C#Cc1c(C)c(Oc2cccc(C(=O)N3CCN(c4ncc(C(F)(F)F)cn4)CC3)c2)n[nH]c1=O]

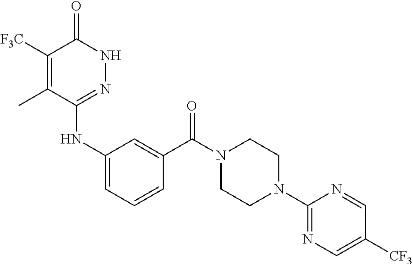 [SMILES: Cc1c(Nc2cccc(C(=O)N3CCN(c4ncc(C(F)(F)F)cn4)CC3)c2)n[nH]c(=O)c1C(F)(F)F]

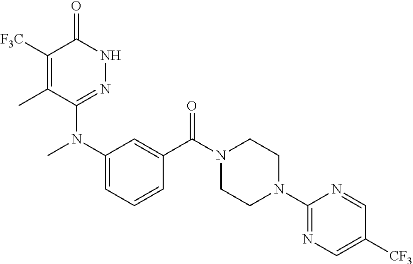 [SMILES: Cc1c(N(C)c2cccc(C(=O)N3CCN(c4ncc(C(F)(F)F)cn4)CC3)c2)n[nH]c(=O)c1C(F)(F)F]

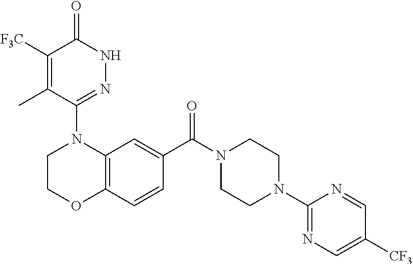 [SMILES: Cc1c(N2CCOc3ccc(C(=O)N4CCN(c5ncc(C(F)(F)F)cn5)CC4)cc32)n[nH]c(=O)c1C(F)(F)F]

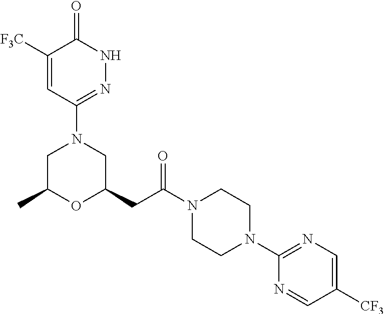 [SMILES: C[C@H]1CN(c2cc(C(F)(F)F)c(=O)[nH]n2)C[C@@H](CC(=O)N2CCN(c3ncc(C(F)(F)F)cn3)CC2)O1]

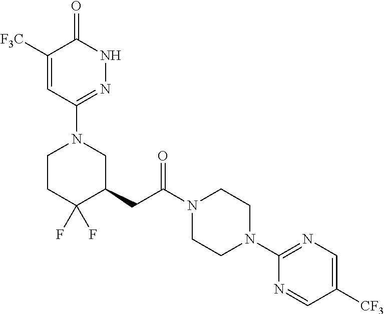 [SMILES: O=C(C[C@@H]1CN(c2cc(C(F)(F)F)c(=O)[nH]n2)CCC1(F)F)N1CCN(c2ncc(C(F)(F)F)cn2)CC1]

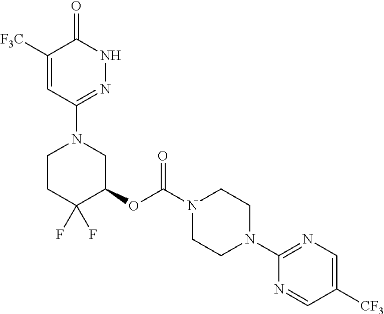 [SMILES: O=C(O[C@@H]1CN(c2cc(C(F)(F)F)c(=O)[nH]n2)CCC1(F)F)N1CCN(c2ncc(C(F)(F)F)cn2)CC1]

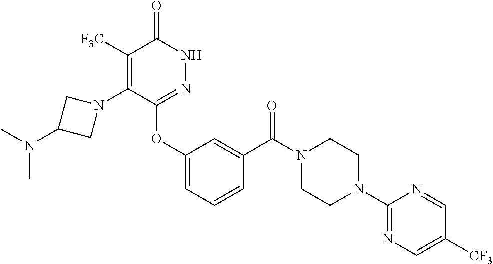 [SMILES: CN(C)C1CN(c2c(Oc3cccc(C(=O)N4CCN(c5ncc(C(F)(F)F)cn5)CC4)c3)n[nH]c(=O)c2C(F)(F)F)C1]

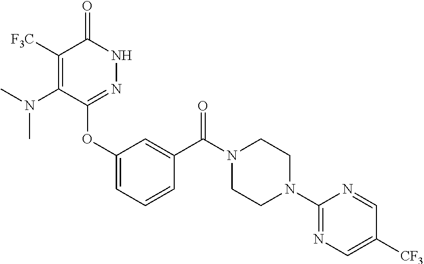 [SMILES: CN(C)c1c(Oc2cccc(C(=O)N3CCN(c4ncc(C(F)(F)F)cn4)CC3)c2)n[nH]c(=O)c1C(F)(F)F]

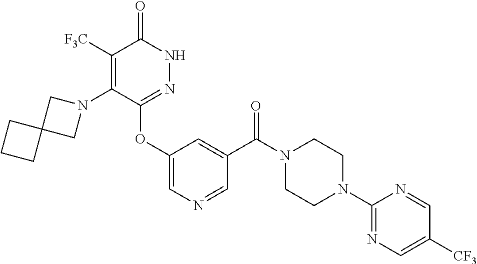 [SMILES: O=C(c1cncc(Oc2n[nH]c(=O)c(C(F)(F)F)c2N2CC3(CCC3)C2)c1)N1CCN(c2ncc(C(F)(F)F)cn2)CC1]

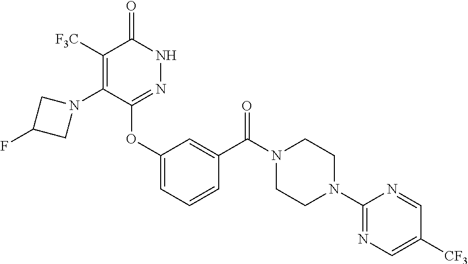 [SMILES: O=C(c1cccc(Oc2n[nH]c(=O)c(C(F)(F)F)c2N2CC(F)C2)c1)N1CCN(c2ncc(C(F)(F)F)cn2)CC1]

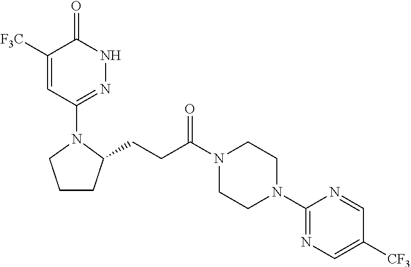 [SMILES: O=C(CC[C@@H]1CCCN1c1cc(C(F)(F)F)c(=O)[nH]n1)N1CCN(c2ncc(C(F)(F)F)cn2)CC1]

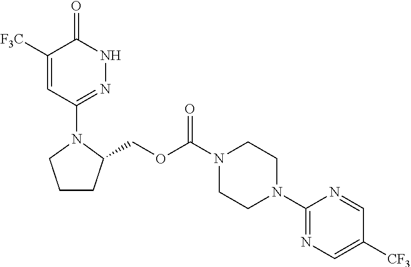 [SMILES: O=C(OC[C@@H]1CCCN1c1cc(C(F)(F)F)c(=O)[nH]n1)N1CCN(c2ncc(C(F)(F)F)cn2)CC1]

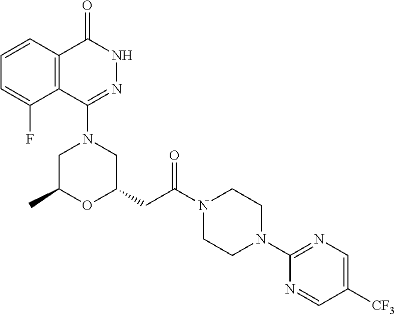 [SMILES: C[C@H]1CN(c2n[nH]c(=O)c3cccc(F)c23)C[C@H](CC(=O)N2CCN(c3ncc(C(F)(F)F)cn3)CC2)O1]

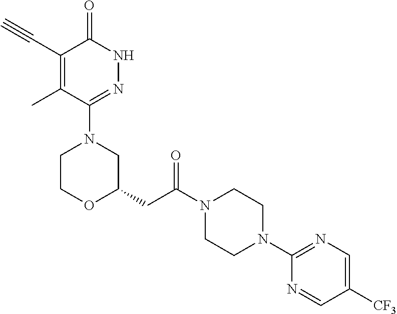 [SMILES: C#Cc1c(C)c(N2CCO[C@@H](CC(=O)N3CCN(c4ncc(C(F)(F)F)cn4)CC3)C2)n[nH]c1=O]

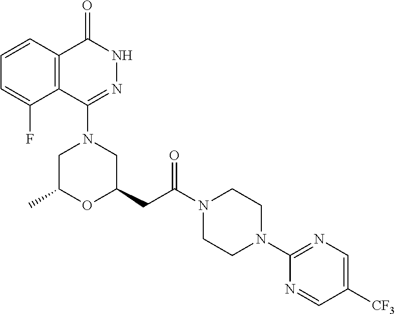 [SMILES: C[C@@H]1CN(c2n[nH]c(=O)c3cccc(F)c23)C[C@@H](CC(=O)N2CCN(c3ncc(C(F)(F)F)cn3)CC2)O1]

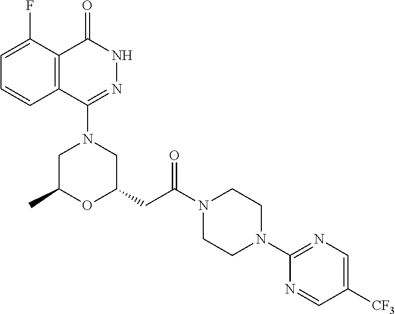 [SMILES: C[C@H]1CN(c2n[nH]c(=O)c3c(F)cccc23)C[C@H](CC(=O)N2CCN(c3ncc(C(F)(F)F)cn3)CC2)O1]

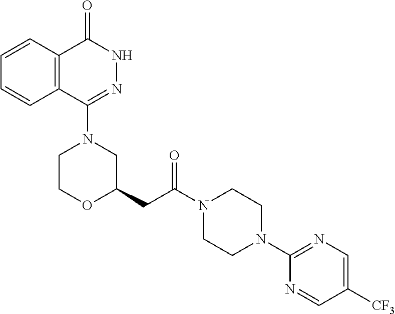 [SMILES: O=C(C[C@@H]1CN(c2n[nH]c(=O)c3ccccc23)CCO1)N1CCN(c2ncc(C(F)(F)F)cn2)CC1]

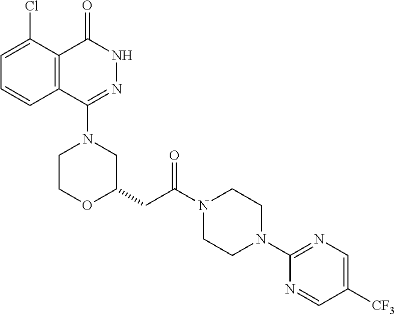 [SMILES: O=C(C[C@H]1CN(c2n[nH]c(=O)c3c(Cl)cccc23)CCO1)N1CCN(c2ncc(C(F)(F)F)cn2)CC1]

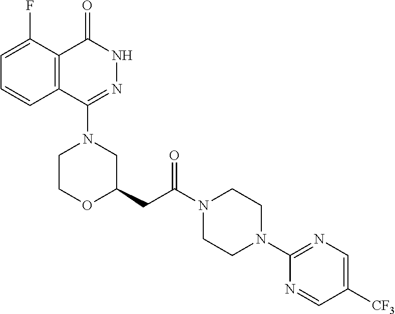 [SMILES: O=C(C[C@@H]1CN(c2n[nH]c(=O)c3c(F)cccc23)CCO1)N1CCN(c2ncc(C(F)(F)F)cn2)CC1]

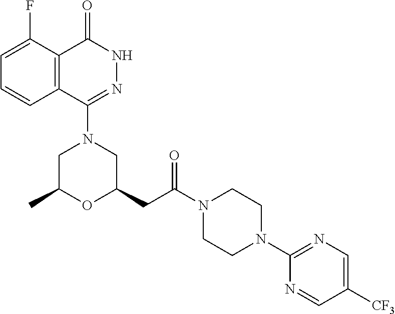 [SMILES: C[C@H]1CN(c2n[nH]c(=O)c3c(F)cccc23)C[C@@H](CC(=O)N2CCN(c3ncc(C(F)(F)F)cn3)CC2)O1]

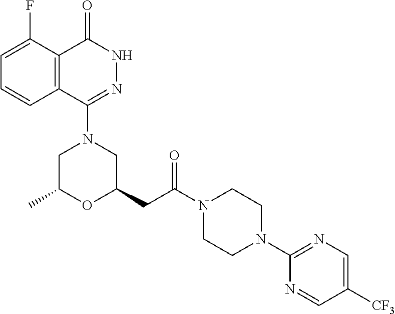 [SMILES: C[C@@H]1CN(c2n[nH]c(=O)c3c(F)cccc23)C[C@@H](CC(=O)N2CCN(c3ncc(C(F)(F)F)cn3)CC2)O1]

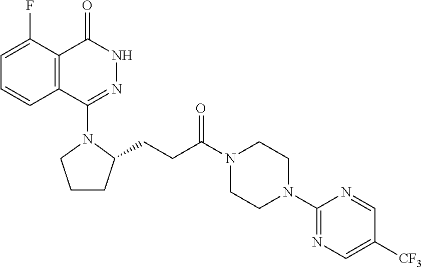 [SMILES: O=C(CC[C@@H]1CCCN1c1n[nH]c(=O)c2c(F)cccc12)N1CCN(c2ncc(C(F)(F)F)cn2)CC1]

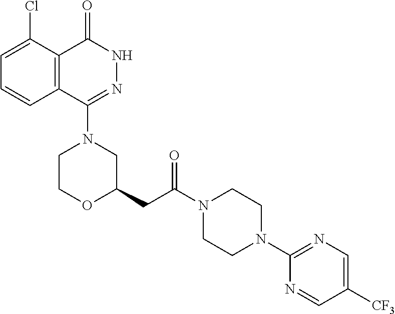 [SMILES: O=C(C[C@@H]1CN(c2n[nH]c(=O)c3c(Cl)cccc23)CCO1)N1CCN(c2ncc(C(F)(F)F)cn2)CC1]

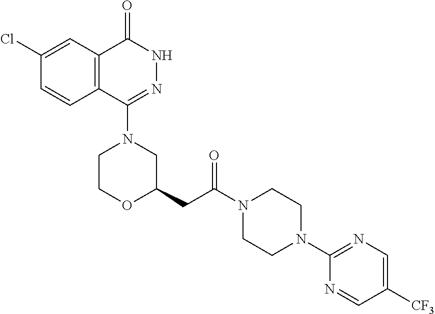 [SMILES: O=C(C[C@@H]1CN(c2n[nH]c(=O)c3cc(Cl)ccc23)CCO1)N1CCN(c2ncc(C(F)(F)F)cn2)CC1]

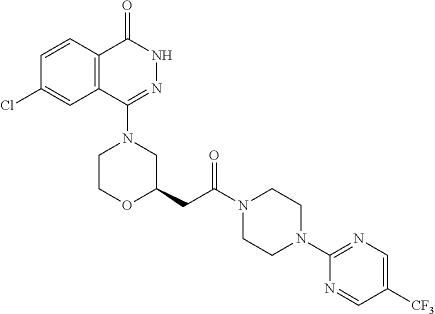 [SMILES: O=C(C[C@@H]1CN(c2n[nH]c(=O)c3ccc(Cl)cc23)CCO1)N1CCN(c2ncc(C(F)(F)F)cn2)CC1]

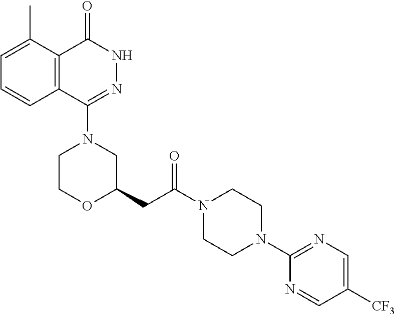 [SMILES: Cc1cccc2c(N3CCO[C@H](CC(=O)N4CCN(c5ncc(C(F)(F)F)cn5)CC4)C3)n[nH]c(=O)c12]

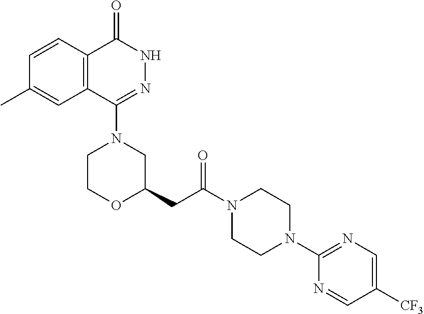 [SMILES: Cc1ccc2c(=O)[nH]nc(N3CCO[C@H](CC(=O)N4CCN(c5ncc(C(F)(F)F)cn5)CC4)C3)c2c1]

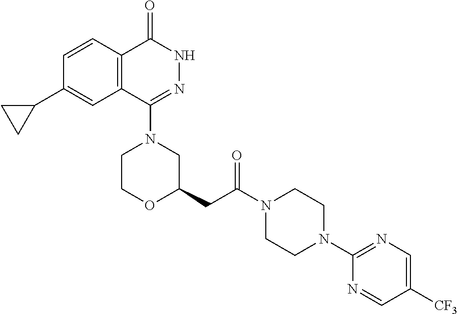 [SMILES: O=C(C[C@@H]1CN(c2n[nH]c(=O)c3ccc(C4CC4)cc23)CCO1)N1CCN(c2ncc(C(F)(F)F)cn2)CC1]

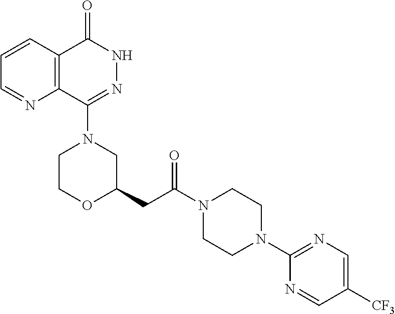 [SMILES: O=C(C[C@@H]1CN(c2n[nH]c(=O)c3cccnc23)CCO1)N1CCN(c2ncc(C(F)(F)F)cn2)CC1]

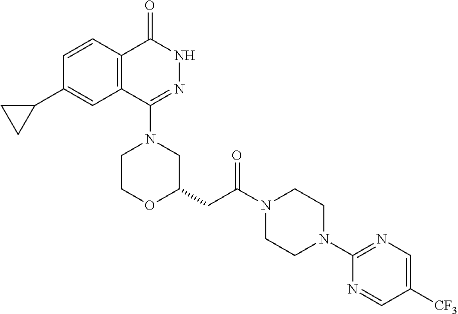 [SMILES: O=C(C[C@H]1CN(c2n[nH]c(=O)c3ccc(C4CC4)cc23)CCO1)N1CCN(c2ncc(C(F)(F)F)cn2)CC1]

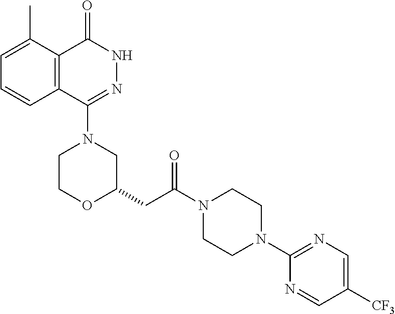 [SMILES: Cc1cccc2c(N3CCO[C@@H](CC(=O)N4CCN(c5ncc(C(F)(F)F)cn5)CC4)C3)n[nH]c(=O)c12]

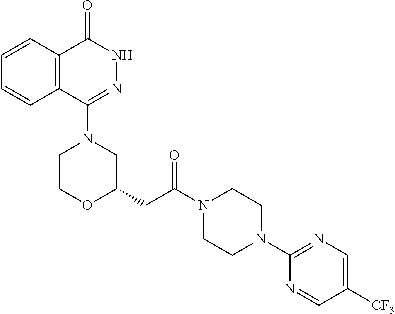 [SMILES: O=C(C[C@H]1CN(c2n[nH]c(=O)c3ccccc23)CCO1)N1CCN(c2ncc(C(F)(F)F)cn2)CC1]

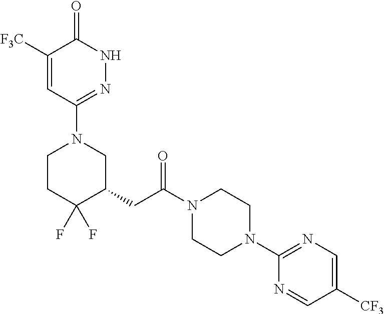 [SMILES: O=C(C[C@H]1CN(c2cc(C(F)(F)F)c(=O)[nH]n2)CCC1(F)F)N1CCN(c2ncc(C(F)(F)F)cn2)CC1]

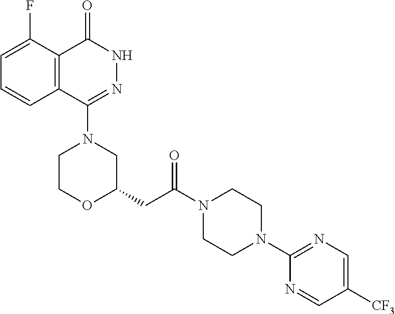 [SMILES: O=C(C[C@H]1CN(c2n[nH]c(=O)c3c(F)cccc23)CCO1)N1CCN(c2ncc(C(F)(F)F)cn2)CC1]

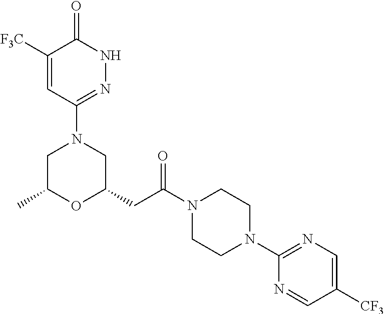 [SMILES: C[C@@H]1CN(c2cc(C(F)(F)F)c(=O)[nH]n2)C[C@H](CC(=O)N2CCN(c3ncc(C(F)(F)F)cn3)CC2)O1]

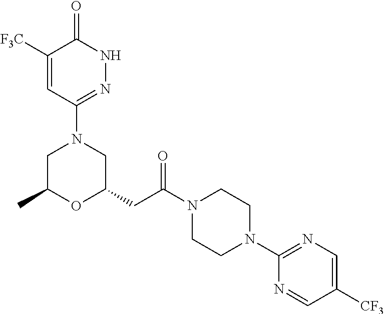 [SMILES: C[C@H]1CN(c2cc(C(F)(F)F)c(=O)[nH]n2)C[C@H](CC(=O)N2CCN(c3ncc(C(F)(F)F)cn3)CC2)O1]

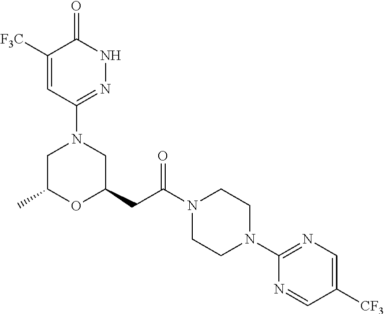 [SMILES: C[C@@H]1CN(c2cc(C(F)(F)F)c(=O)[nH]n2)C[C@@H](CC(=O)N2CCN(c3ncc(C(F)(F)F)cn3)CC2)O1]

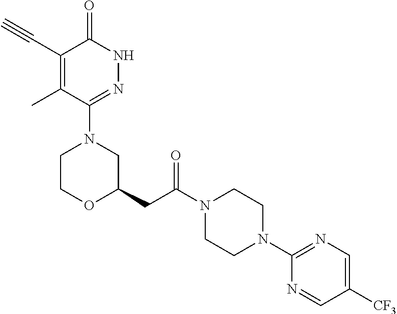 [SMILES: C#Cc1c(C)c(N2CCO[C@H](CC(=O)N3CCN(c4ncc(C(F)(F)F)cn4)CC3)C2)n[nH]c1=O]

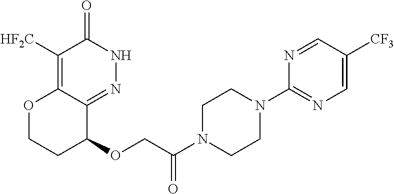

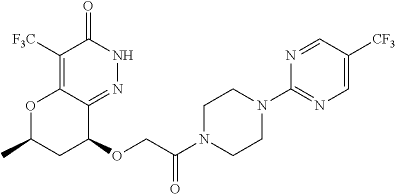 [SMILES: C[C@@H]1C[C@H](OCC(=O)N2CCN(c3ncc(C(F)(F)F)cn3)CC2)c2n[nH]c(=O)c(C(F)(F)F)c2O1]

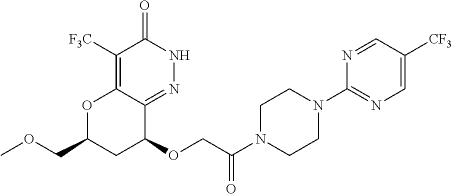 [SMILES: COC[C@@H]1C[C@H](OCC(=O)N2CCN(c3ncc(C(F)(F)F)cn3)CC2)c2n[nH]c(=O)c(C(F)(F)F)c2O1]

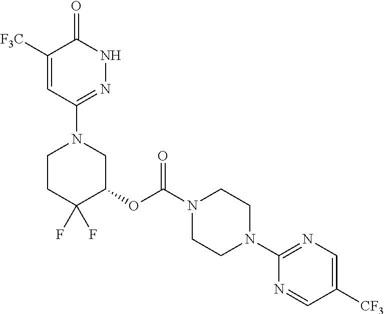 [SMILES: O=C(O[C@H]1CN(c2cc(C(F)(F)F)c(=O)[nH]n2)CCC1(F)F)N1CCN(c2ncc(C(F)(F)F)cn2)CC1]

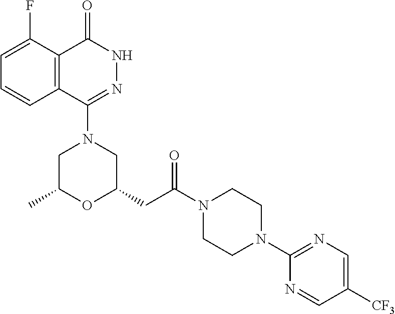 [SMILES: C[C@@H]1CN(c2n[nH]c(=O)c3c(F)cccc23)C[C@H](CC(=O)N2CCN(c3ncc(C(F)(F)F)cn3)CC2)O1]

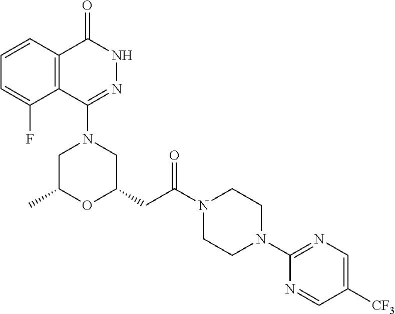 [SMILES: C[C@@H]1CN(c2n[nH]c(=O)c3cccc(F)c23)C[C@H](CC(=O)N2CCN(c3ncc(C(F)(F)F)cn3)CC2)O1]

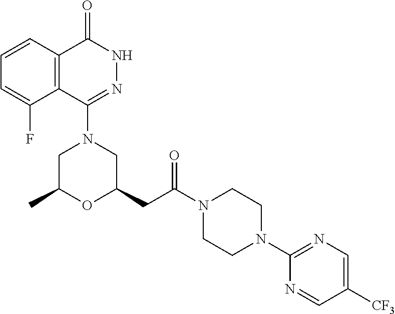 [SMILES: C[C@H]1CN(c2n[nH]c(=O)c3cccc(F)c23)C[C@@H](CC(=O)N2CCN(c3ncc(C(F)(F)F)cn3)CC2)O1]

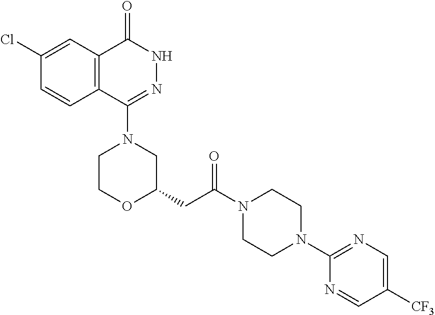 [SMILES: O=C(C[C@H]1CN(c2n[nH]c(=O)c3cc(Cl)ccc23)CCO1)N1CCN(c2ncc(C(F)(F)F)cn2)CC1]

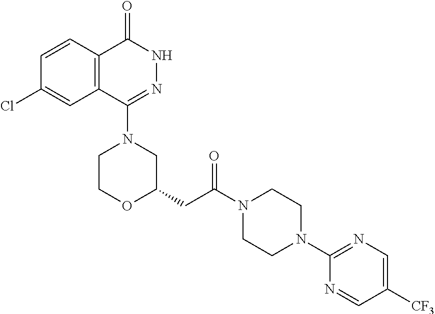 [SMILES: O=C(C[C@H]1CN(c2n[nH]c(=O)c3ccc(Cl)cc23)CCO1)N1CCN(c2ncc(C(F)(F)F)cn2)CC1]

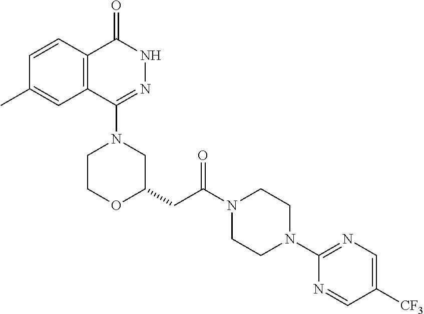 [SMILES: Cc1ccc2c(=O)[nH]nc(N3CCO[C@@H](CC(=O)N4CCN(c5ncc(C(F)(F)F)cn5)CC4)C3)c2c1]

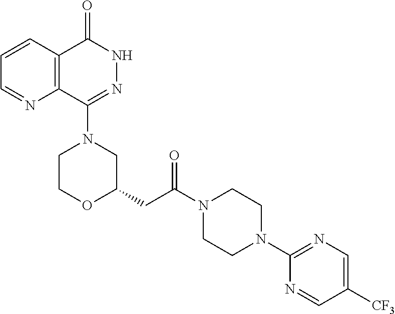 [SMILES: O=C(C[C@H]1CN(c2n[nH]c(=O)c3cccnc23)CCO1)N1CCN(c2ncc(C(F)(F)F)cn2)CC1]

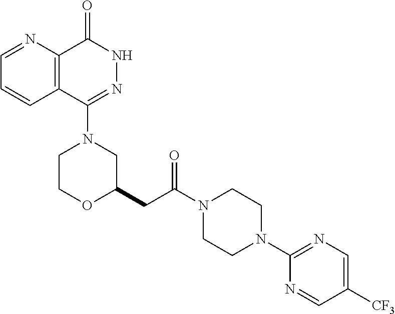 [SMILES: O=C(C[C@@H]1CN(c2n[nH]c(=O)c3ncccc23)CCO1)N1CCN(c2ncc(C(F)(F)F)cn2)CC1]

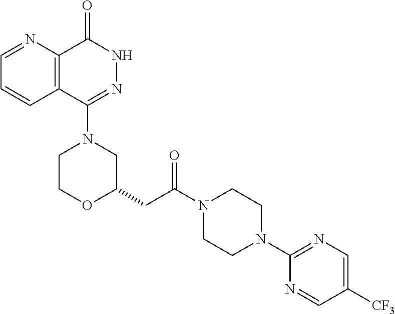 [SMILES: O=C(C[C@H]1CN(c2n[nH]c(=O)c3ncccc23)CCO1)N1CCN(c2ncc(C(F)(F)F)cn2)CC1]

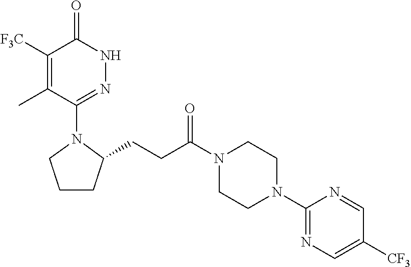 [SMILES: Cc1c(N2CCC[C@H]2CCC(=O)N2CCN(c3ncc(C(F)(F)F)cn3)CC2)n[nH]c(=O)c1C(F)(F)F]

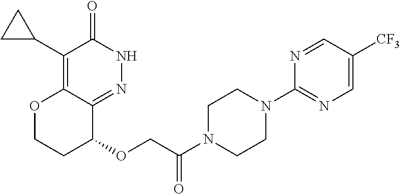 [SMILES: O=C(CO[C@@H]1CCOc2c1n[nH]c(=O)c2C1CC1)N1CCN(c2ncc(C(F)(F)F)cn2)CC1]

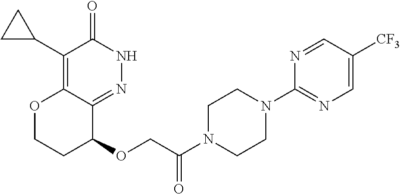 [SMILES: O=C(CO[C@H]1CCOc2c1n[nH]c(=O)c2C1CC1)N1CCN(c2ncc(C(F)(F)F)cn2)CC1]

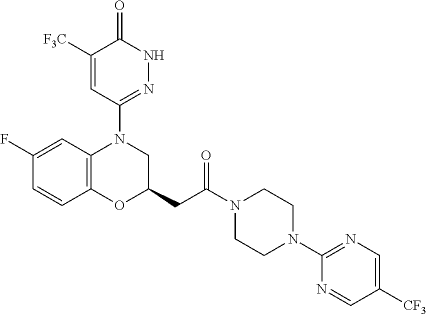 [SMILES: O=C(C[C@@H]1CN(c2cc(C(F)(F)F)c(=O)[nH]n2)c2cc(F)ccc2O1)N1CCN(c2ncc(C(F)(F)F)cn2)CC1]

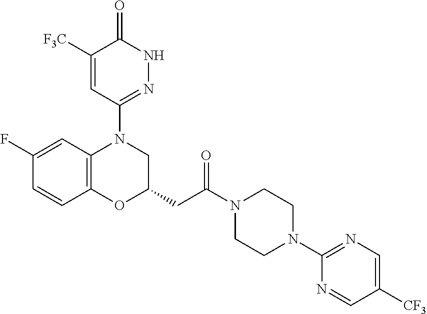 [SMILES: O=C(C[C@H]1CN(c2cc(C(F)(F)F)c(=O)[nH]n2)c2cc(F)ccc2O1)N1CCN(c2ncc(C(F)(F)F)cn2)CC1]

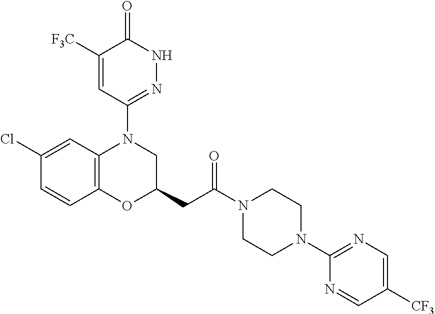 [SMILES: O=C(C[C@@H]1CN(c2cc(C(F)(F)F)c(=O)[nH]n2)c2cc(Cl)ccc2O1)N1CCN(c2ncc(C(F)(F)F)cn2)CC1]

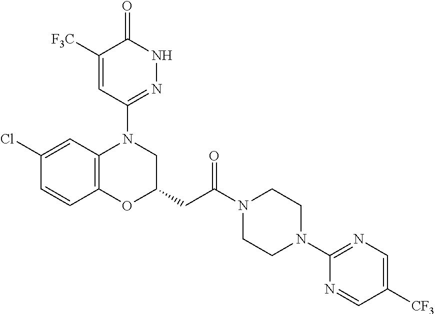 [SMILES: O=C(C[C@H]1CN(c2cc(C(F)(F)F)c(=O)[nH]n2)c2cc(Cl)ccc2O1)N1CCN(c2ncc(C(F)(F)F)cn2)CC1]

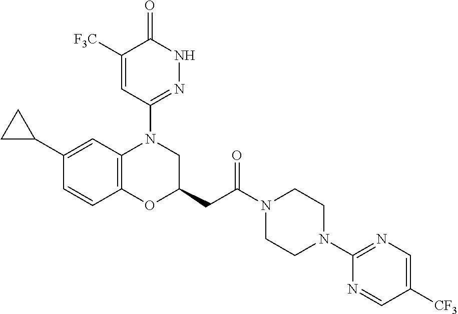 [SMILES: O=C(C[C@@H]1CN(c2cc(C(F)(F)F)c(=O)[nH]n2)c2cc(C3CC3)ccc2O1)N1CCN(c2ncc(C(F)(F)F)cn2)CC1]

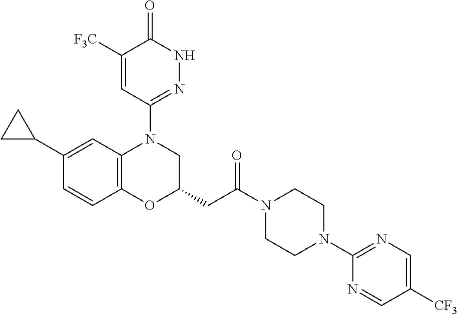 [SMILES: O=C(C[C@H]1CN(c2cc(C(F)(F)F)c(=O)[nH]n2)c2cc(C3CC3)ccc2O1)N1CCN(c2ncc(C(F)(F)F)cn2)CC1]

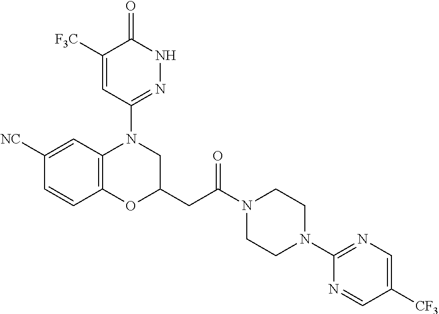 [SMILES: N#Cc1ccc2c(c1)N(c1cc(C(F)(F)F)c(=O)[nH]n1)CC(CC(=O)N1CCN(c3ncc(C(F)(F)F)cn3)CC1)O2]

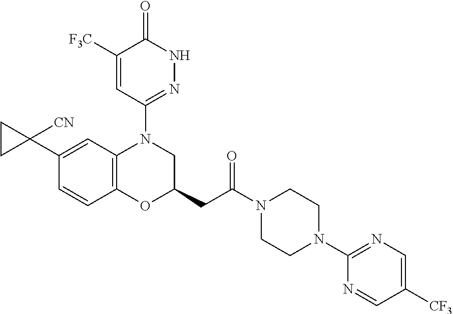 [SMILES: N#CC1(c2ccc3c(c2)N(c2cc(C(F)(F)F)c(=O)[nH]n2)C[C@@H](CC(=O)N2CCN(c4ncc(C(F)(F)F)cn4)CC2)O3)CC1]

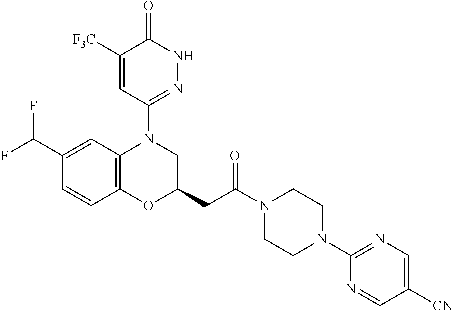 [SMILES: N#Cc1cnc(N2CCN(C(=O)C[C@@H]3CN(c4cc(C(F)(F)F)c(=O)[nH]n4)c4cc(C(F)F)ccc4O3)CC2)nc1]

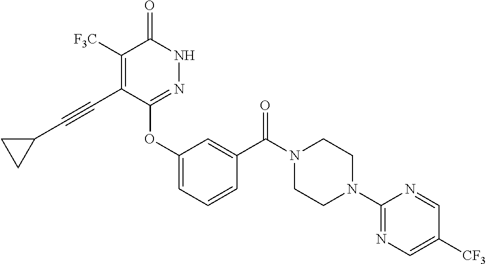 [SMILES: O=C(c1cccc(Oc2n[nH]c(=O)c(C(F)(F)F)c2C#CC2CC2)c1)N1CCN(c2ncc(C(F)(F)F)cn2)CC1]

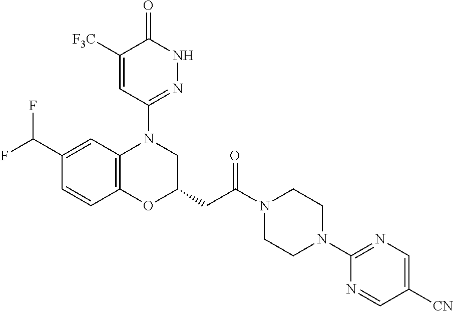 [SMILES: N#Cc1cnc(N2CCN(C(=O)C[C@H]3CN(c4cc(C(F)(F)F)c(=O)[nH]n4)c4cc(C(F)F)ccc4O3)CC2)nc1]

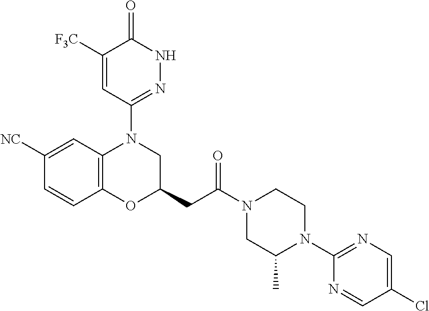 [SMILES: C[C@@H]1CN(C(=O)C[C@@H]2CN(c3cc(C(F)(F)F)c(=O)[nH]n3)c3cc(C#N)ccc3O2)CCN1c1ncc(Cl)cn1]

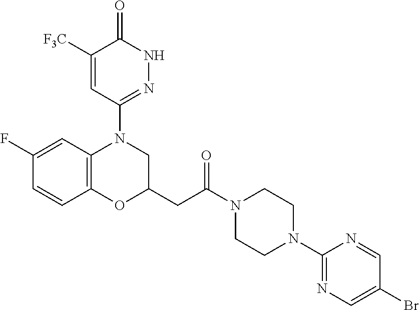 [SMILES: O=C(CC1CN(c2cc(C(F)(F)F)c(=O)[nH]n2)c2cc(F)ccc2O1)N1CCN(c2ncc(Br)cn2)CC1]

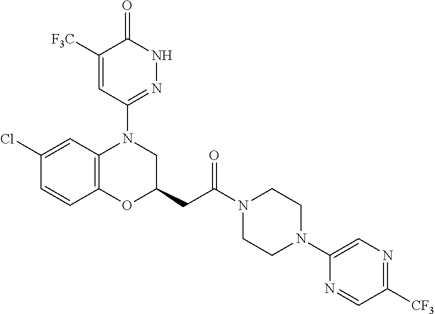 [SMILES: O=C(C[C@@H]1CN(c2cc(C(F)(F)F)c(=O)[nH]n2)c2cc(Cl)ccc2O1)N1CCN(c2cnc(C(F)(F)F)cn2)CC1]

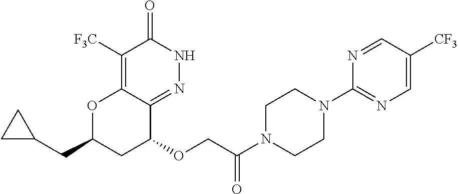 [SMILES: O=C(CO[C@@H]1C[C@@H](CC2CC2)Oc2c1n[nH]c(=O)c2C(F)(F)F)N1CCN(c2ncc(C(F)(F)F)cn2)CC1]

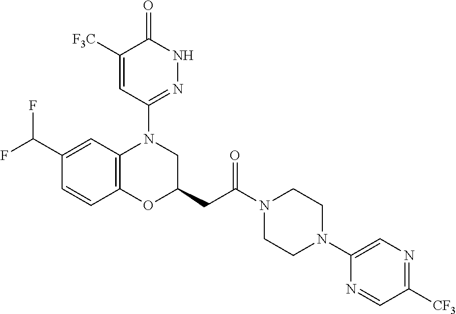 [SMILES: O=C(C[C@@H]1CN(c2cc(C(F)(F)F)c(=O)[nH]n2)c2cc(C(F)F)ccc2O1)N1CCN(c2cnc(C(F)(F)F)cn2)CC1]

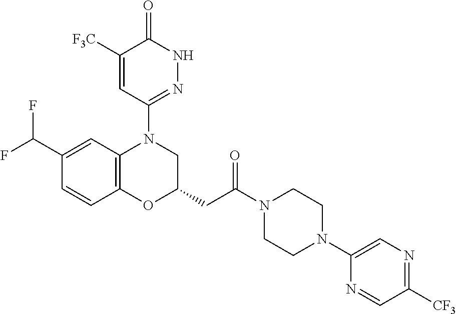 [SMILES: O=C(C[C@H]1CN(c2cc(C(F)(F)F)c(=O)[nH]n2)c2cc(C(F)F)ccc2O1)N1CCN(c2cnc(C(F)(F)F)cn2)CC1]

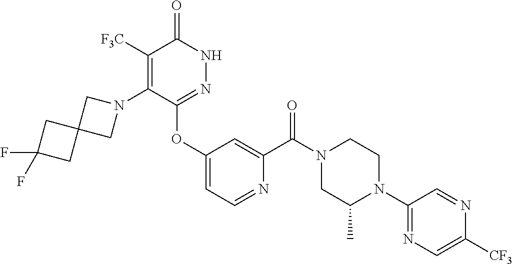 [SMILES: C[C@@H]1CN(C(=O)c2cc(Oc3n[nH]c(=O)c(C(F)(F)F)c3N3CC4(C3)CC(F)(F)C4)ccn2)CCN1c1cnc(C(F)(F)F)cn1]

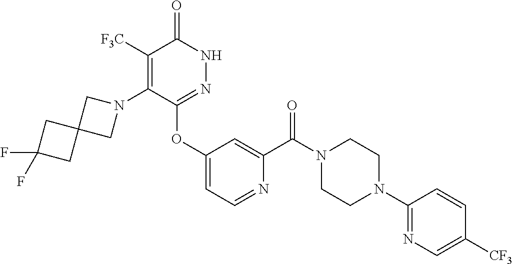 [SMILES: O=C(c1cc(Oc2n[nH]c(=O)c(C(F)(F)F)c2N2CC3(C2)CC(F)(F)C3)ccn1)N1CCN(c2ccc(C(F)(F)F)cn2)CC1]

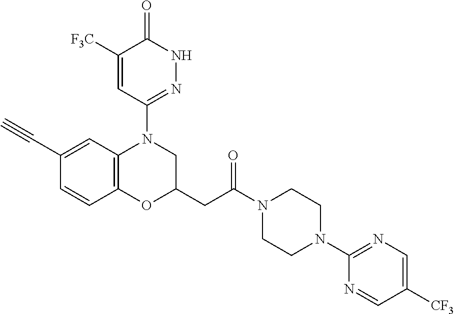 [SMILES: C#Cc1ccc2c(c1)N(c1cc(C(F)(F)F)c(=O)[nH]n1)CC(CC(=O)N1CCN(c3ncc(C(F)(F)F)cn3)CC1)O2]

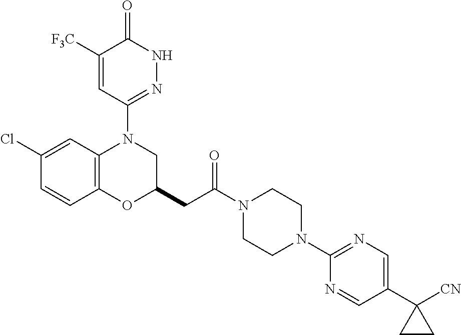 [SMILES: N#CC1(c2cnc(N3CCN(C(=O)C[C@@H]4CN(c5cc(C(F)(F)F)c(=O)[nH]n5)c5cc(Cl)ccc5O4)CC3)nc2)CC1]

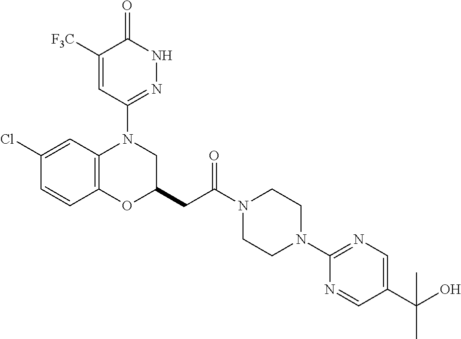 [SMILES: CC(C)(O)c1cnc(N2CCN(C(=O)C[C@@H]3CN(c4cc(C(F)(F)F)c(=O)[nH]n4)c4cc(Cl)ccc4O3)CC2)nc1]

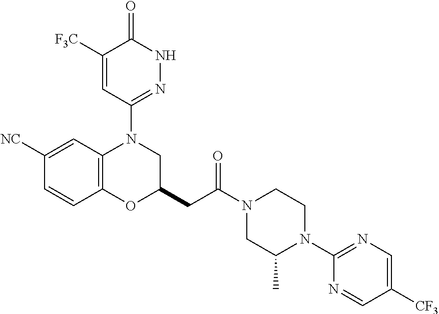 [SMILES: C[C@@H]1CN(C(=O)C[C@@H]2CN(c3cc(C(F)(F)F)c(=O)[nH]n3)c3cc(C#N)ccc3O2)CCN1c1ncc(C(F)(F)F)cn1]

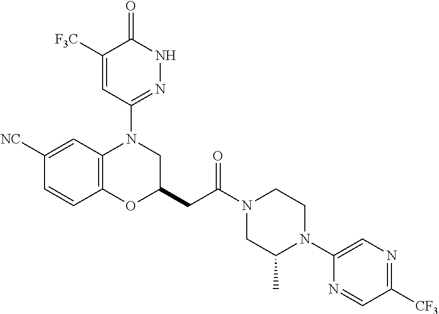 [SMILES: C[C@@H]1CN(C(=O)C[C@@H]2CN(c3cc(C(F)(F)F)c(=O)[nH]n3)c3cc(C#N)ccc3O2)CCN1c1cnc(C(F)(F)F)cn1]

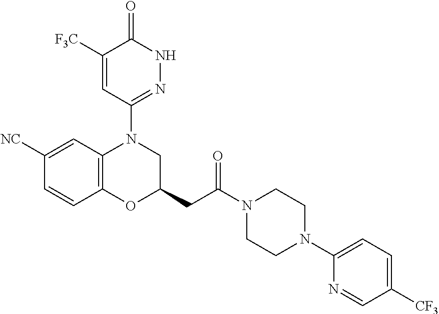 [SMILES: N#Cc1ccc2c(c1)N(c1cc(C(F)(F)F)c(=O)[nH]n1)C[C@@H](CC(=O)N1CCN(c3ccc(C(F)(F)F)cn3)CC1)O2]

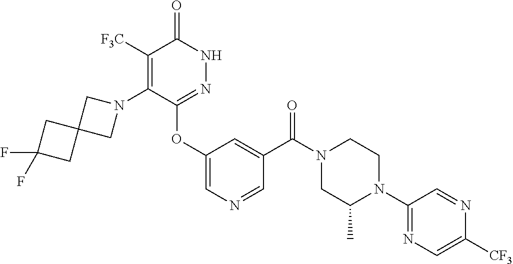 [SMILES: C[C@@H]1CN(C(=O)c2cncc(Oc3n[nH]c(=O)c(C(F)(F)F)c3N3CC4(C3)CC(F)(F)C4)c2)CCN1c1cnc(C(F)(F)F)cn1]

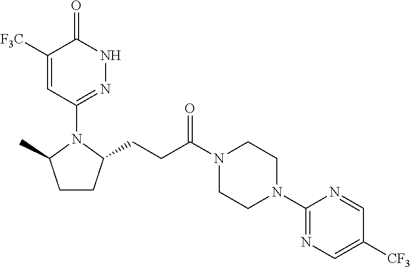 [SMILES: C[C@@H]1CC[C@@H](CCC(=O)N2CCN(c3ncc(C(F)(F)F)cn3)CC2)N1c1cc(C(F)(F)F)c(=O)[nH]n1]

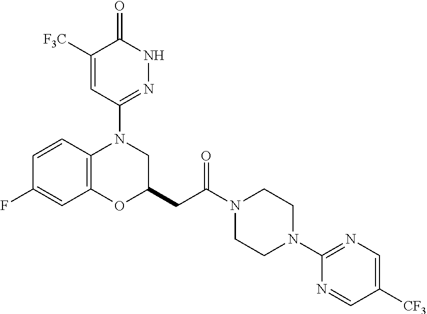 [SMILES: O=C(C[C@@H]1CN(c2cc(C(F)(F)F)c(=O)[nH]n2)c2ccc(F)cc2O1)N1CCN(c2ncc(C(F)(F)F)cn2)CC1]

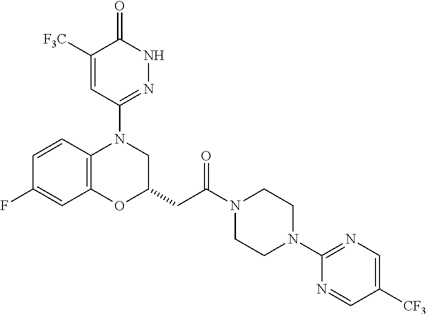 [SMILES: O=C(C[C@H]1CN(c2cc(C(F)(F)F)c(=O)[nH]n2)c2ccc(F)cc2O1)N1CCN(c2ncc(C(F)(F)F)cn2)CC1]

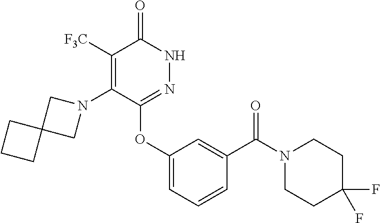 [SMILES: O=C(c1cccc(Oc2n[nH]c(=O)c(C(F)(F)F)c2N2CC3(CCC3)C2)c1)N1CCC(F)(F)CC1]

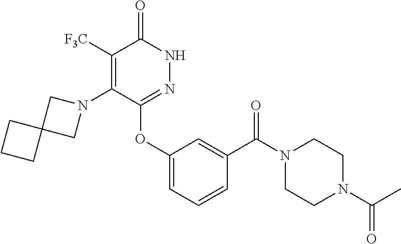 [SMILES: CC(=O)N1CCN(C(=O)c2cccc(Oc3n[nH]c(=O)c(C(F)(F)F)c3N3CC4(CCC4)C3)c2)CC1]

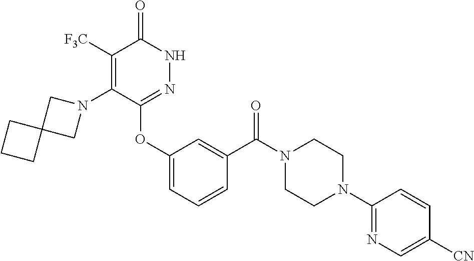 [SMILES: N#Cc1ccc(N2CCN(C(=O)c3cccc(Oc4n[nH]c(=O)c(C(F)(F)F)c4N4CC5(CCC5)C4)c3)CC2)nc1]

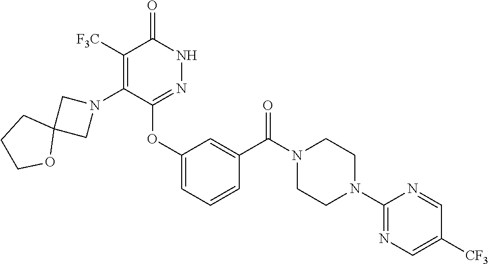 [SMILES: O=C(c1cccc(Oc2n[nH]c(=O)c(C(F)(F)F)c2N2CC3(CCCO3)C2)c1)N1CCN(c2ncc(C(F)(F)F)cn2)CC1]

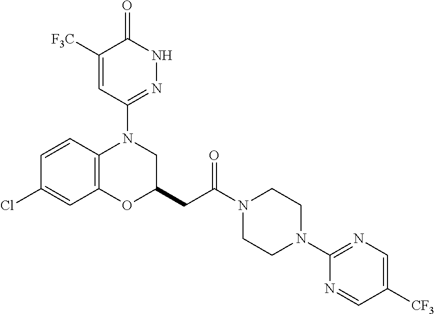 [SMILES: O=C(C[C@@H]1CN(c2cc(C(F)(F)F)c(=O)[nH]n2)c2ccc(Cl)cc2O1)N1CCN(c2ncc(C(F)(F)F)cn2)CC1]

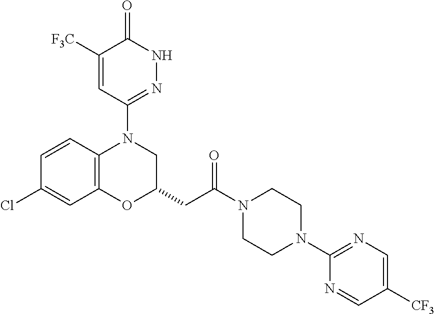 [SMILES: O=C(C[C@H]1CN(c2cc(C(F)(F)F)c(=O)[nH]n2)c2ccc(Cl)cc2O1)N1CCN(c2ncc(C(F)(F)F)cn2)CC1]

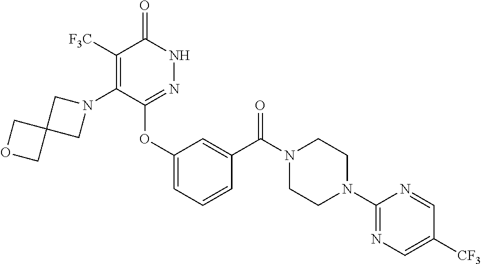 [SMILES: O=C(c1cccc(Oc2n[nH]c(=O)c(C(F)(F)F)c2N2CC3(COC3)C2)c1)N1CCN(c2ncc(C(F)(F)F)cn2)CC1]

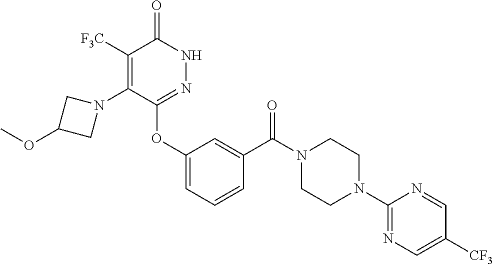 [SMILES: COC1CN(c2c(Oc3cccc(C(=O)N4CCN(c5ncc(C(F)(F)F)cn5)CC4)c3)n[nH]c(=O)c2C(F)(F)F)C1]

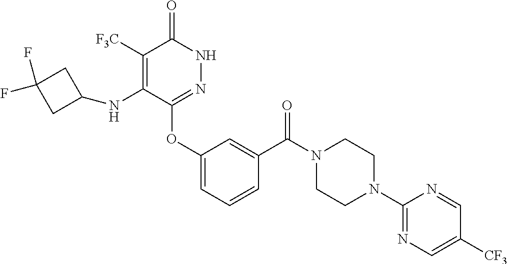 [SMILES: O=C(c1cccc(Oc2n[nH]c(=O)c(C(F)(F)F)c2NC2CC(F)(F)C2)c1)N1CCN(c2ncc(C(F)(F)F)cn2)CC1]

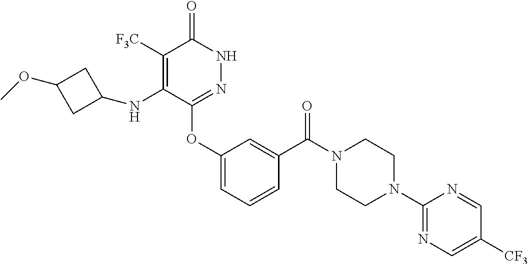 [SMILES: COC1CC(Nc2c(Oc3cccc(C(=O)N4CCN(c5ncc(C(F)(F)F)cn5)CC4)c3)n[nH]c(=O)c2C(F)(F)F)C1]

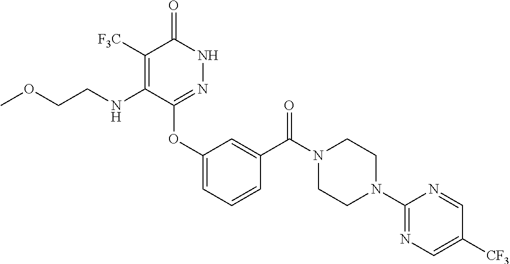 [SMILES: COCCNc1c(Oc2cccc(C(=O)N3CCN(c4ncc(C(F)(F)F)cn4)CC3)c2)n[nH]c(=O)c1C(F)(F)F]

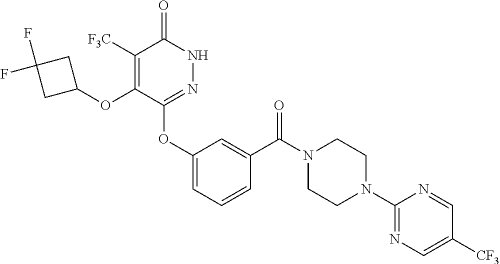 [SMILES: O=C(c1cccc(Oc2n[nH]c(=O)c(C(F)(F)F)c2OC2CC(F)(F)C2)c1)N1CCN(c2ncc(C(F)(F)F)cn2)CC1]

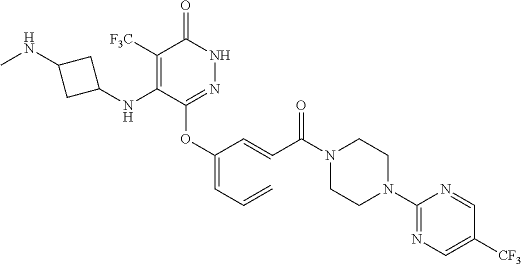 [SMILES: C=C/C=C(\C=C\C(=O)N1CCN(c2ncc(C(F)(F)F)cn2)CC1)Oc1n[nH]c(=O)c(C(F)(F)F)c1NC1CC(NC)C1]

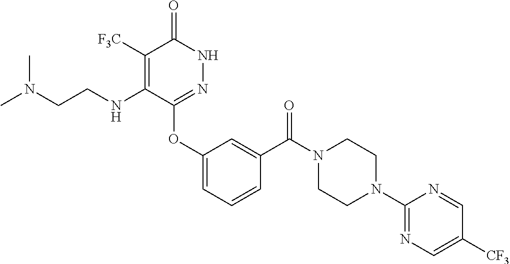 [SMILES: CN(C)CCNc1c(Oc2cccc(C(=O)N3CCN(c4ncc(C(F)(F)F)cn4)CC3)c2)n[nH]c(=O)c1C(F)(F)F]

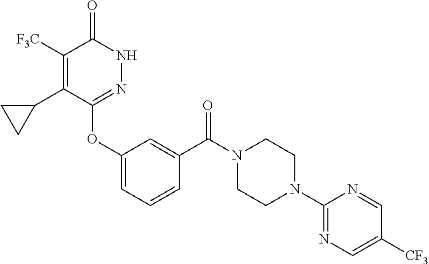 [SMILES: O=C(c1cccc(Oc2n[nH]c(=O)c(C(F)(F)F)c2C2CC2)c1)N1CCN(c2ncc(C(F)(F)F)cn2)CC1]

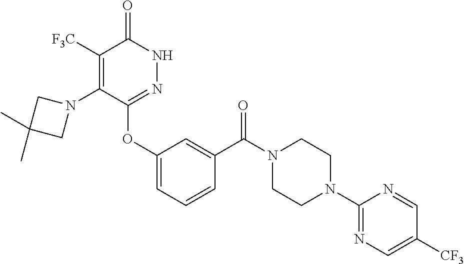 [SMILES: CC1(C)CN(c2c(Oc3cccc(C(=O)N4CCN(c5ncc(C(F)(F)F)cn5)CC4)c3)n[nH]c(=O)c2C(F)(F)F)C1]

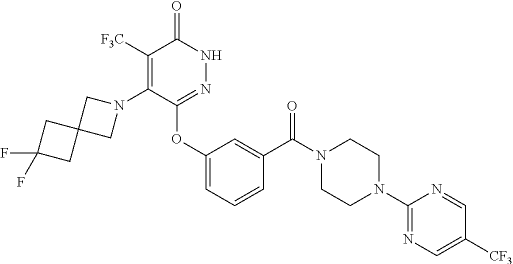 [SMILES: O=C(c1cccc(Oc2n[nH]c(=O)c(C(F)(F)F)c2N2CC3(C2)CC(F)(F)C3)c1)N1CCN(c2ncc(C(F)(F)F)cn2)CC1]

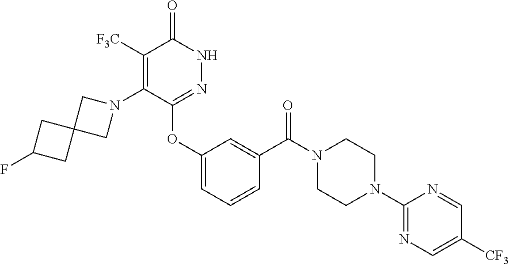 [SMILES: O=C(c1cccc(Oc2n[nH]c(=O)c(C(F)(F)F)c2N2CC3(CC(F)C3)C2)c1)N1CCN(c2ncc(C(F)(F)F)cn2)CC1]

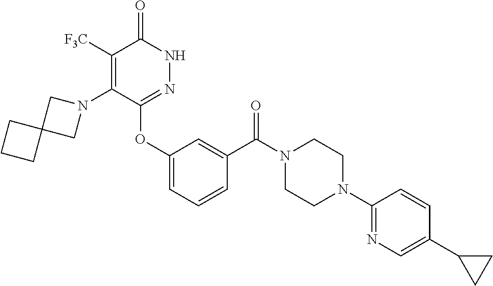 [SMILES: O=C(c1cccc(Oc2n[nH]c(=O)c(C(F)(F)F)c2N2CC3(CCC3)C2)c1)N1CCN(c2ccc(C3CC3)cn2)CC1]

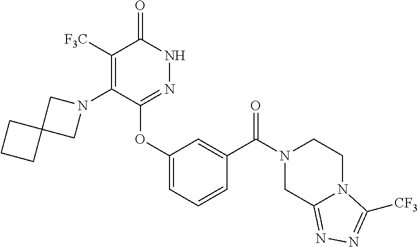 [SMILES: O=C(c1cccc(Oc2n[nH]c(=O)c(C(F)(F)F)c2N2CC3(CCC3)C2)c1)N1CCn2c(nnc2C(F)(F)F)C1]

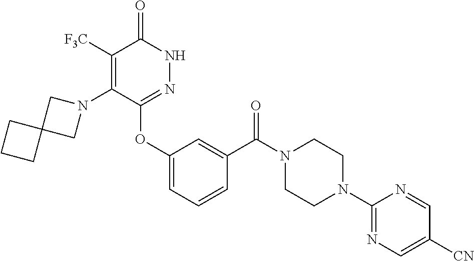 [SMILES: N#Cc1cnc(N2CCN(C(=O)c3cccc(Oc4n[nH]c(=O)c(C(F)(F)F)c4N4CC5(CCC5)C4)c3)CC2)nc1]

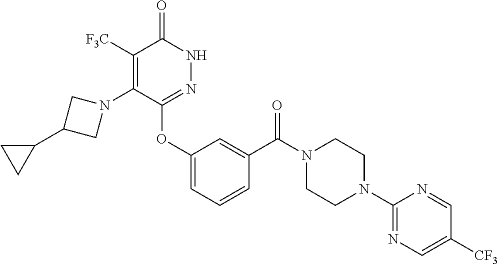 [SMILES: O=C(c1cccc(Oc2n[nH]c(=O)c(C(F)(F)F)c2N2CC(C3CC3)C2)c1)N1CCN(c2ncc(C(F)(F)F)cn2)CC1]

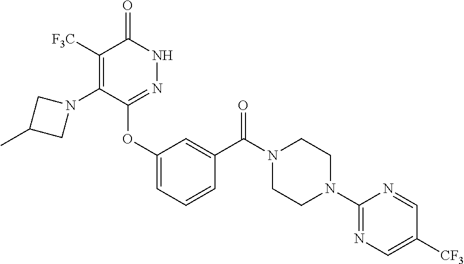 [SMILES: CC1CN(c2c(Oc3cccc(C(=O)N4CCN(c5ncc(C(F)(F)F)cn5)CC4)c3)n[nH]c(=O)c2C(F)(F)F)C1]

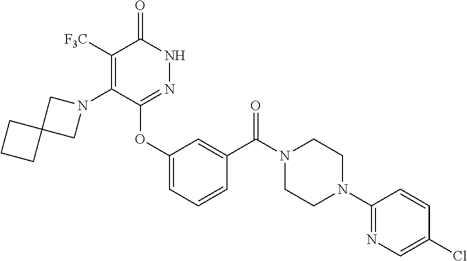 [SMILES: O=C(c1cccc(Oc2n[nH]c(=O)c(C(F)(F)F)c2N2CC3(CCC3)C2)c1)N1CCN(c2ccc(Cl)cn2)CC1]

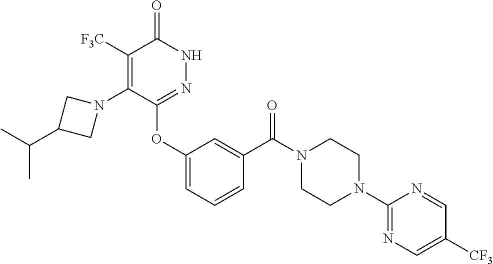 [SMILES: CC(C)C1CN(c2c(Oc3cccc(C(=O)N4CCN(c5ncc(C(F)(F)F)cn5)CC4)c3)n[nH]c(=O)c2C(F)(F)F)C1]

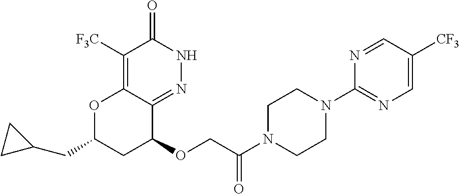 [SMILES: O=C(CO[C@H]1C[C@H](CC2CC2)Oc2c1n[nH]c(=O)c2C(F)(F)F)N1CCN(c2ncc(C(F)(F)F)cn2)CC1]

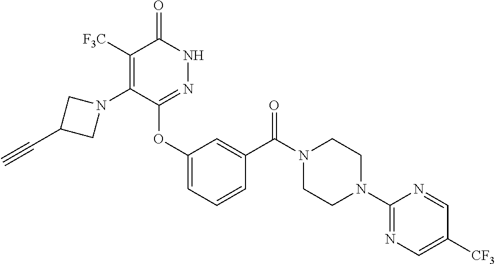 [SMILES: C#CC1CN(c2c(Oc3cccc(C(=O)N4CCN(c5ncc(C(F)(F)F)cn5)CC4)c3)n[nH]c(=O)c2C(F)(F)F)C1]

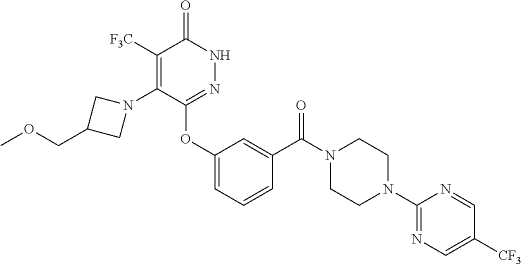 [SMILES: COCC1CN(c2c(Oc3cccc(C(=O)N4CCN(c5ncc(C(F)(F)F)cn5)CC4)c3)n[nH]c(=O)c2C(F)(F)F)C1]

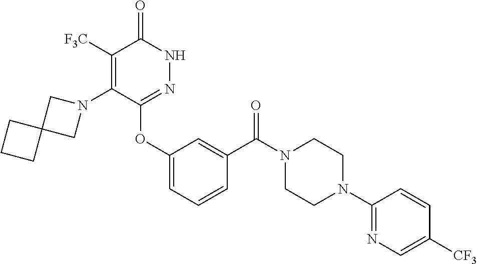 [SMILES: O=C(c1cccc(Oc2n[nH]c(=O)c(C(F)(F)F)c2N2CC3(CCC3)C2)c1)N1CCN(c2ccc(C(F)(F)F)cn2)CC1]

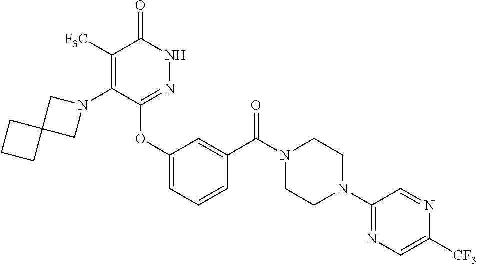 [SMILES: O=C(c1cccc(Oc2n[nH]c(=O)c(C(F)(F)F)c2N2CC3(CCC3)C2)c1)N1CCN(c2cnc(C(F)(F)F)cn2)CC1]

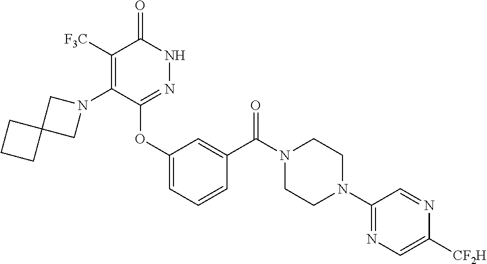 [SMILES: O=C(c1cccc(Oc2n[nH]c(=O)c(C(F)(F)F)c2N2CC3(CCC3)C2)c1)N1CCN(c2cnc(C(F)F)cn2)CC1]

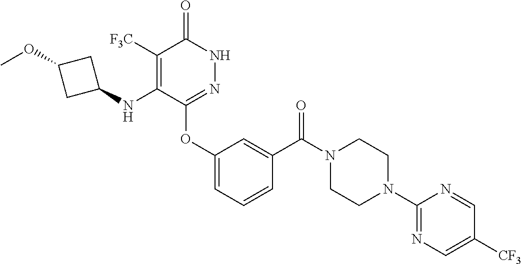 [SMILES: CO[C@H]1C[C@H](Nc2c(Oc3cccc(C(=O)N4CCN(c5ncc(C(F)(F)F)cn5)CC4)c3)n[nH]c(=O)c2C(F)(F)F)C1]

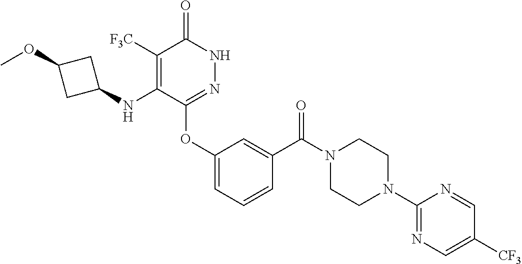 [SMILES: CO[C@H]1C[C@@H](Nc2c(Oc3cccc(C(=O)N4CCN(c5ncc(C(F)(F)F)cn5)CC4)c3)n[nH]c(=O)c2C(F)(F)F)C1]

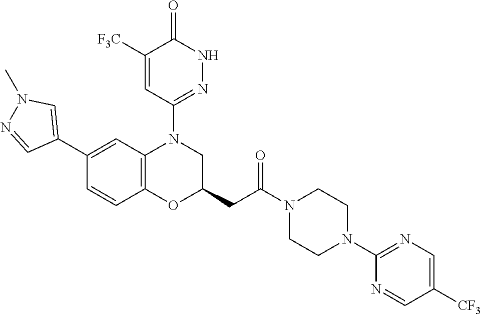 [SMILES: Cn1cc(-c2ccc3c(c2)N(c2cc(C(F)(F)F)c(=O)[nH]n2)C[C@@H](CC(=O)N2CCN(c4ncc(C(F)(F)F)cn4)CC2)O3)cn1]

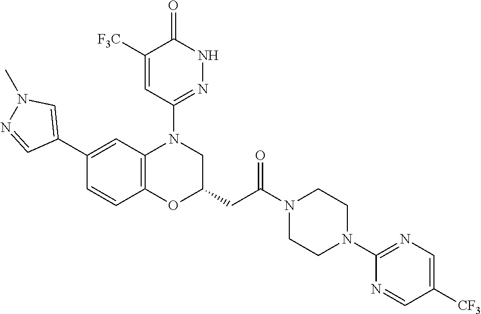 [SMILES: Cn1cc(-c2ccc3c(c2)N(c2cc(C(F)(F)F)c(=O)[nH]n2)C[C@H](CC(=O)N2CCN(c4ncc(C(F)(F)F)cn4)CC2)O3)cn1]

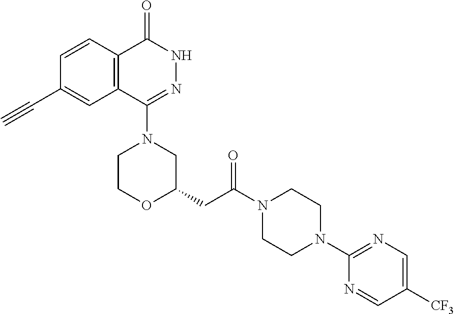 [SMILES: C#Cc1ccc2c(=O)[nH]nc(N3CCO[C@@H](CC(=O)N4CCN(c5ncc(C(F)(F)F)cn5)CC4)C3)c2c1]

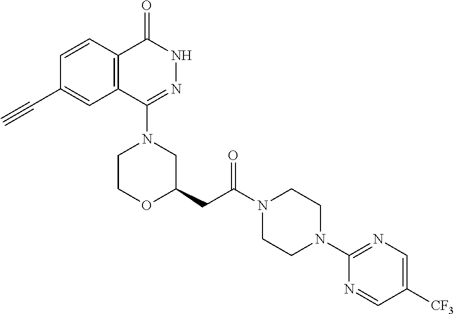 [SMILES: C#Cc1ccc2c(=O)[nH]nc(N3CCO[C@H](CC(=O)N4CCN(c5ncc(C(F)(F)F)cn5)CC4)C3)c2c1]

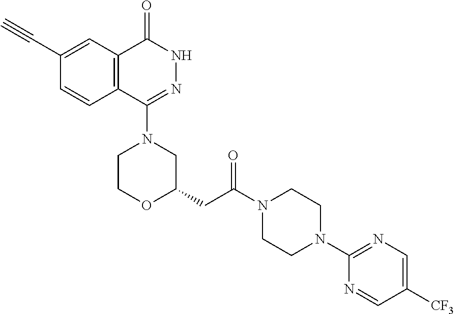 [SMILES: C#Cc1ccc2c(N3CCO[C@@H](CC(=O)N4CCN(c5ncc(C(F)(F)F)cn5)CC4)C3)n[nH]c(=O)c2c1]

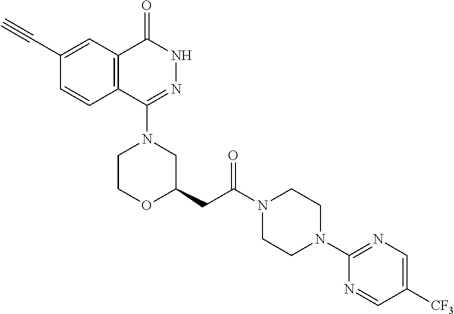 [SMILES: C#Cc1ccc2c(N3CCO[C@H](CC(=O)N4CCN(c5ncc(C(F)(F)F)cn5)CC4)C3)n[nH]c(=O)c2c1]

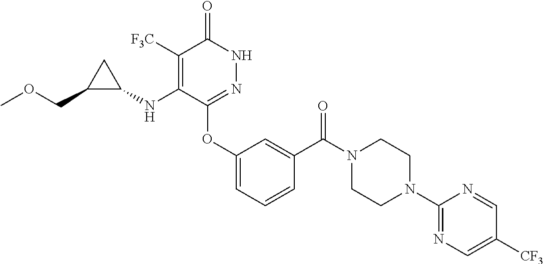 [SMILES: COC[C@H]1C[C@@H]1Nc1c(Oc2cccc(C(=O)N3CCN(c4ncc(C(F)(F)F)cn4)CC3)c2)n[nH]c(=O)c1C(F)(F)F]

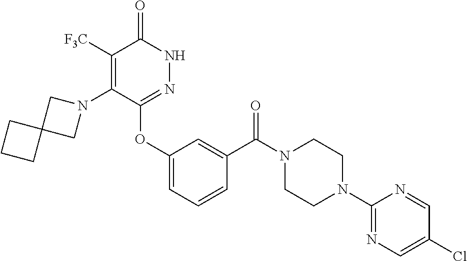 [SMILES: O=C(c1cccc(Oc2n[nH]c(=O)c(C(F)(F)F)c2N2CC3(CCC3)C2)c1)N1CCN(c2ncc(Cl)cn2)CC1]

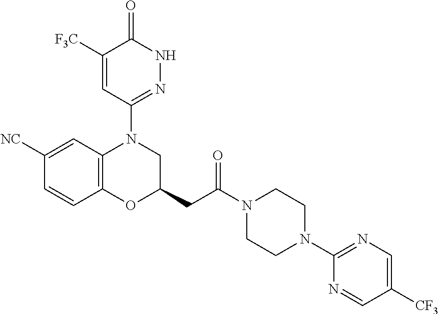 [SMILES: N#Cc1ccc2c(c1)N(c1cc(C(F)(F)F)c(=O)[nH]n1)C[C@@H](CC(=O)N1CCN(c3ncc(C(F)(F)F)cn3)CC1)O2]

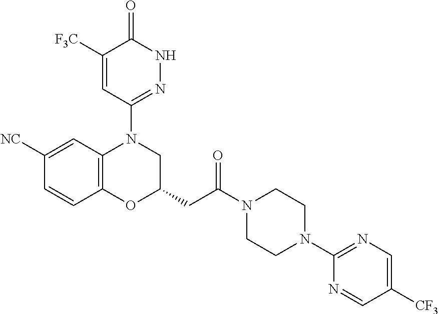 [SMILES: N#Cc1ccc2c(c1)N(c1cc(C(F)(F)F)c(=O)[nH]n1)C[C@H](CC(=O)N1CCN(c3ncc(C(F)(F)F)cn3)CC1)O2]

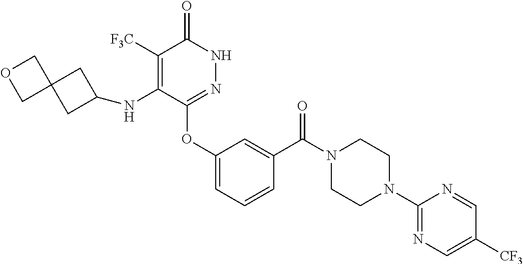 [SMILES: O=C(c1cccc(Oc2n[nH]c(=O)c(C(F)(F)F)c2NC2CC3(COC3)C2)c1)N1CCN(c2ncc(C(F)(F)F)cn2)CC1]

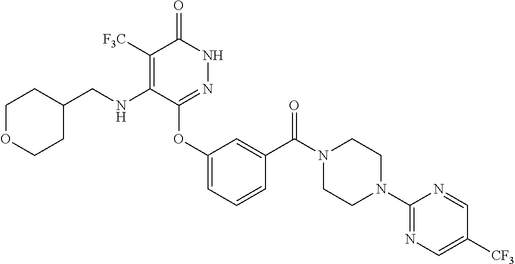 [SMILES: O=C(c1cccc(Oc2n[nH]c(=O)c(C(F)(F)F)c2NCC2CCOCC2)c1)N1CCN(c2ncc(C(F)(F)F)cn2)CC1]

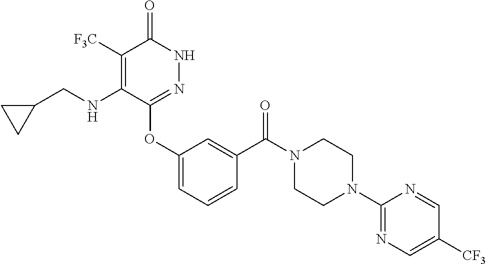 [SMILES: O=C(c1cccc(Oc2n[nH]c(=O)c(C(F)(F)F)c2NCC2CC2)c1)N1CCN(c2ncc(C(F)(F)F)cn2)CC1]

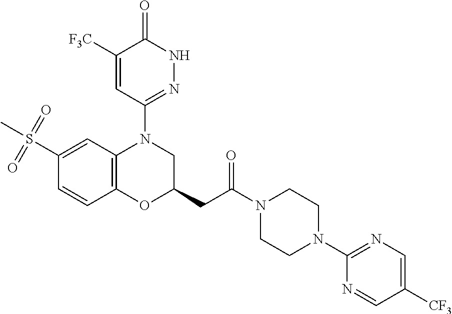 [SMILES: CS(=O)(=O)c1ccc2c(c1)N(c1cc(C(F)(F)F)c(=O)[nH]n1)C[C@@H](CC(=O)N1CCN(c3ncc(C(F)(F)F)cn3)CC1)O2]

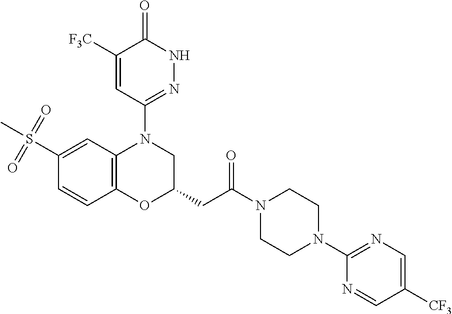 [SMILES: CS(=O)(=O)c1ccc2c(c1)N(c1cc(C(F)(F)F)c(=O)[nH]n1)C[C@H](CC(=O)N1CCN(c3ncc(C(F)(F)F)cn3)CC1)O2]

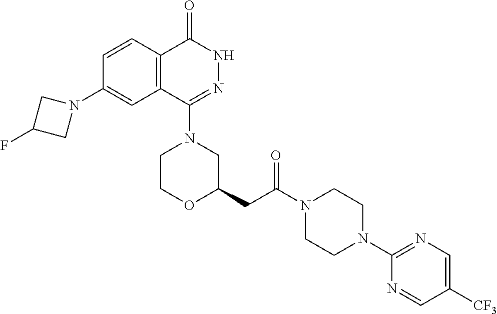 [SMILES: O=C(C[C@@H]1CN(c2n[nH]c(=O)c3ccc(N4CC(F)C4)cc23)CCO1)N1CCN(c2ncc(C(F)(F)F)cn2)CC1]

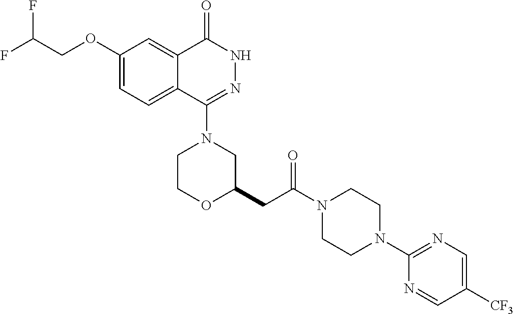 [SMILES: O=C(C[C@@H]1CN(c2n[nH]c(=O)c3cc(OCC(F)F)ccc23)CCO1)N1CCN(c2ncc(C(F)(F)F)cn2)CC1]

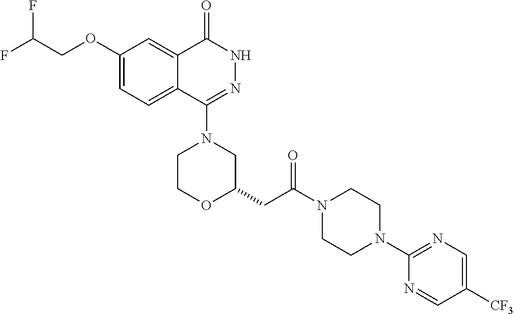 [SMILES: O=C(C[C@H]1CN(c2n[nH]c(=O)c3cc(OCC(F)F)ccc23)CCO1)N1CCN(c2ncc(C(F)(F)F)cn2)CC1]

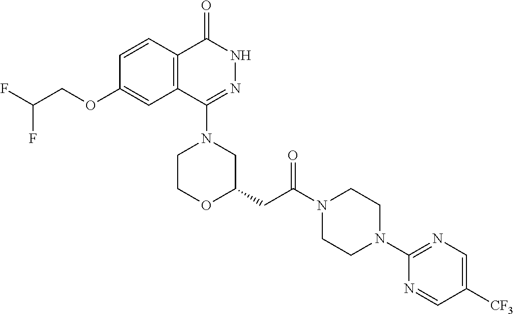 [SMILES: O=C(C[C@H]1CN(c2n[nH]c(=O)c3ccc(OCC(F)F)cc23)CCO1)N1CCN(c2ncc(C(F)(F)F)cn2)CC1]

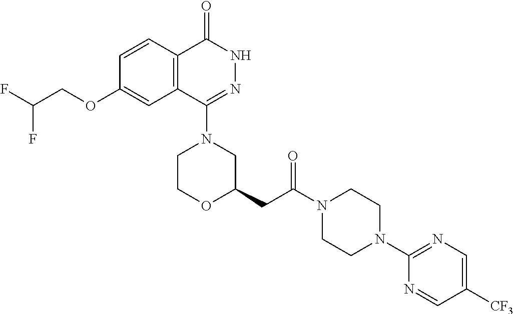 [SMILES: O=C(C[C@@H]1CN(c2n[nH]c(=O)c3ccc(OCC(F)F)cc23)CCO1)N1CCN(c2ncc(C(F)(F)F)cn2)CC1]

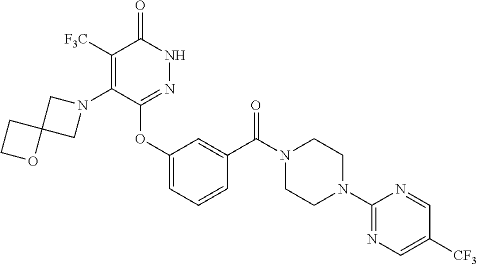 [SMILES: O=C(c1cccc(Oc2n[nH]c(=O)c(C(F)(F)F)c2N2CC3(CCO3)C2)c1)N1CCN(c2ncc(C(F)(F)F)cn2)CC1]

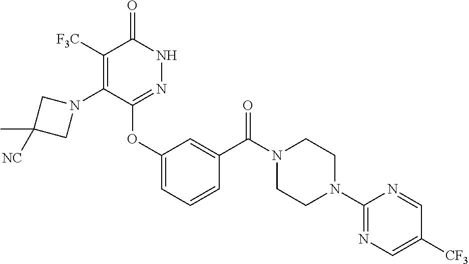 [SMILES: CC1(C#N)CN(c2c(Oc3cccc(C(=O)N4CCN(c5ncc(C(F)(F)F)cn5)CC4)c3)n[nH]c(=O)c2C(F)(F)F)C1]

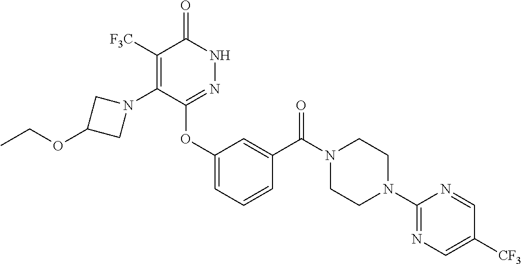 [SMILES: CCOC1CN(c2c(Oc3cccc(C(=O)N4CCN(c5ncc(C(F)(F)F)cn5)CC4)c3)n[nH]c(=O)c2C(F)(F)F)C1]

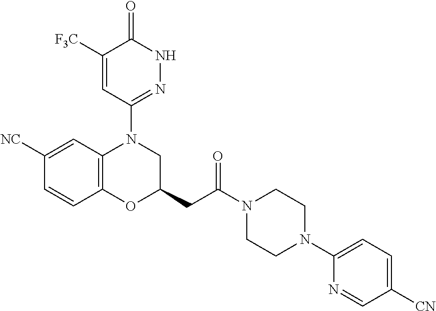 [SMILES: N#Cc1ccc(N2CCN(C(=O)C[C@@H]3CN(c4cc(C(F)(F)F)c(=O)[nH]n4)c4cc(C#N)ccc4O3)CC2)nc1]

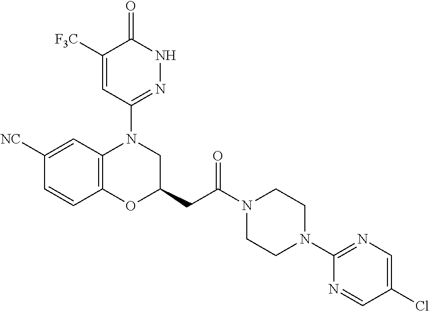 [SMILES: N#Cc1ccc2c(c1)N(c1cc(C(F)(F)F)c(=O)[nH]n1)C[C@@H](CC(=O)N1CCN(c3ncc(Cl)cn3)CC1)O2]

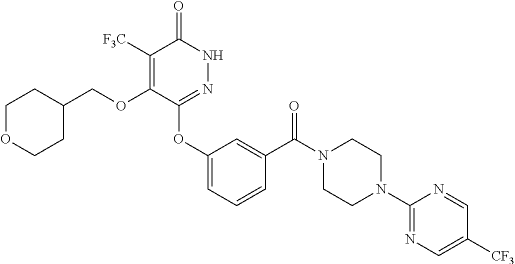 [SMILES: O=C(c1cccc(Oc2n[nH]c(=O)c(C(F)(F)F)c2OCC2CCOCC2)c1)N1CCN(c2ncc(C(F)(F)F)cn2)CC1]

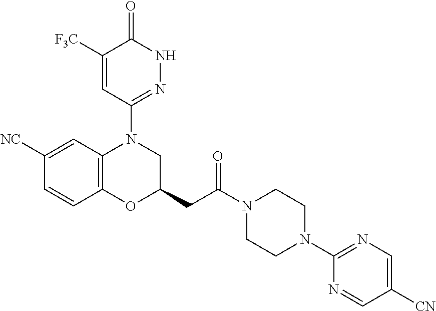 [SMILES: N#Cc1cnc(N2CCN(C(=O)C[C@@H]3CN(c4cc(C(F)(F)F)c(=O)[nH]n4)c4cc(C#N)ccc4O3)CC2)nc1]

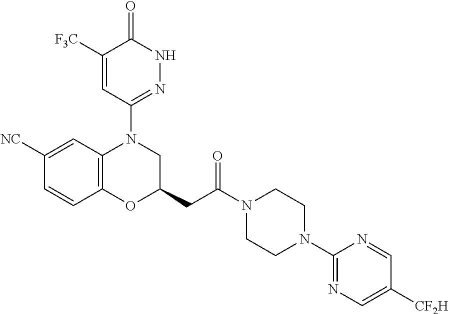 [SMILES: N#Cc1ccc2c(c1)N(c1cc(C(F)(F)F)c(=O)[nH]n1)C[C@@H](CC(=O)N1CCN(c3ncc(C(F)F)cn3)CC1)O2]

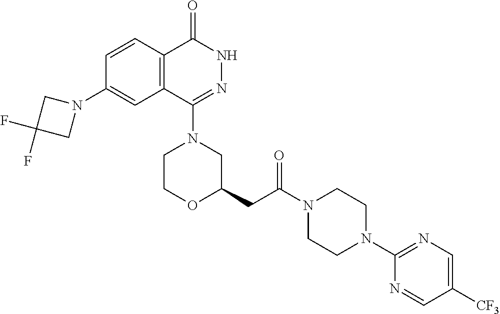 [SMILES: O=C(C[C@@H]1CN(c2n[nH]c(=O)c3ccc(N4CC(F)(F)C4)cc23)CCO1)N1CCN(c2ncc(C(F)(F)F)cn2)CC1]

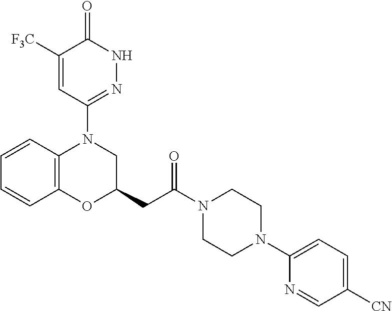 [SMILES: N#Cc1ccc(N2CCN(C(=O)C[C@@H]3CN(c4cc(C(F)(F)F)c(=O)[nH]n4)c4ccccc4O3)CC2)nc1]

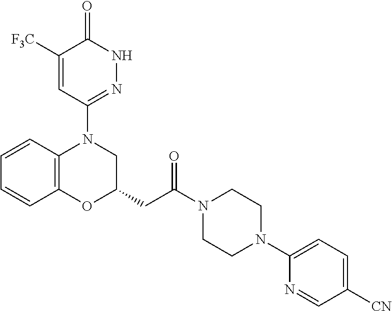 [SMILES: N#Cc1ccc(N2CCN(C(=O)C[C@H]3CN(c4cc(C(F)(F)F)c(=O)[nH]n4)c4ccccc4O3)CC2)nc1]

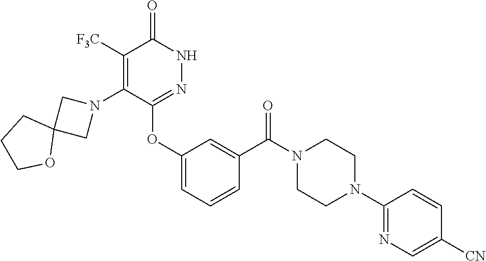 [SMILES: N#Cc1ccc(N2CCN(C(=O)c3cccc(Oc4n[nH]c(=O)c(C(F)(F)F)c4N4CC5(CCCO5)C4)c3)CC2)nc1]

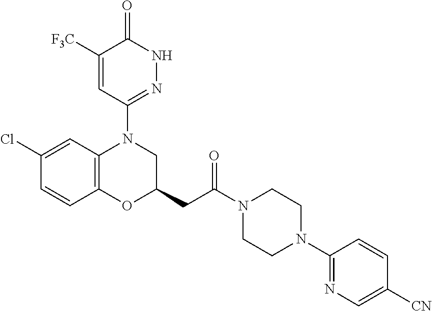 [SMILES: N#Cc1ccc(N2CCN(C(=O)C[C@@H]3CN(c4cc(C(F)(F)F)c(=O)[nH]n4)c4cc(Cl)ccc4O3)CC2)nc1]

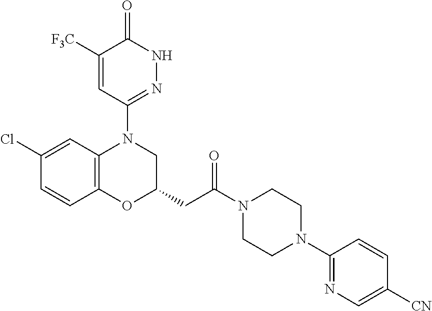 [SMILES: N#Cc1ccc(N2CCN(C(=O)C[C@H]3CN(c4cc(C(F)(F)F)c(=O)[nH]n4)c4cc(Cl)ccc4O3)CC2)nc1]

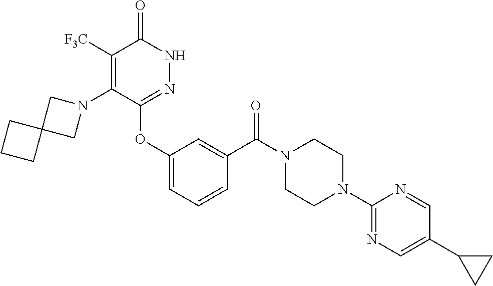 [SMILES: O=C(c1cccc(Oc2n[nH]c(=O)c(C(F)(F)F)c2N2CC3(CCC3)C2)c1)N1CCN(c2ncc(C3CC3)cn2)CC1]

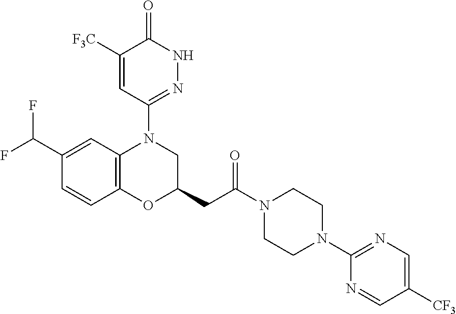 [SMILES: O=C(C[C@@H]1CN(c2cc(C(F)(F)F)c(=O)[nH]n2)c2cc(C(F)F)ccc2O1)N1CCN(c2ncc(C(F)(F)F)cn2)CC1]

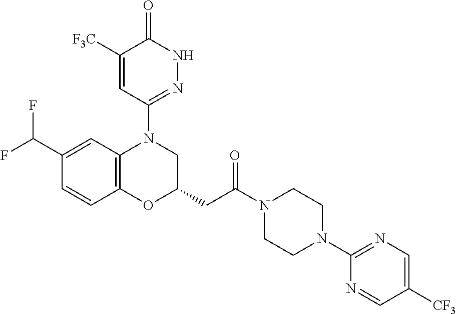 [SMILES: O=C(C[C@H]1CN(c2cc(C(F)(F)F)c(=O)[nH]n2)c2cc(C(F)F)ccc2O1)N1CCN(c2ncc(C(F)(F)F)cn2)CC1]

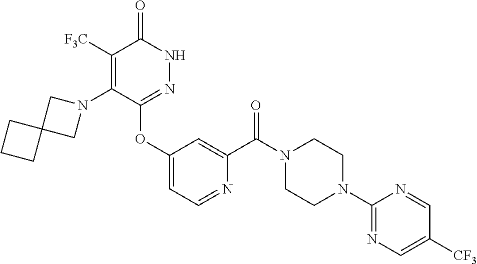 [SMILES: O=C(c1cc(Oc2n[nH]c(=O)c(C(F)(F)F)c2N2CC3(CCC3)C2)ccn1)N1CCN(c2ncc(C(F)(F)F)cn2)CC1]

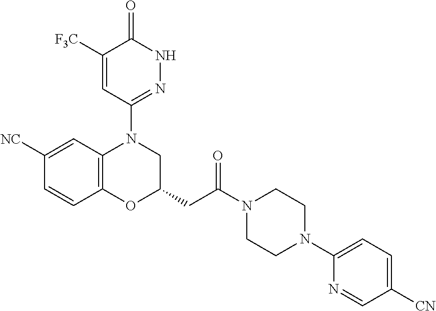 [SMILES: N#Cc1ccc(N2CCN(C(=O)C[C@H]3CN(c4cc(C(F)(F)F)c(=O)[nH]n4)c4cc(C#N)ccc4O3)CC2)nc1]

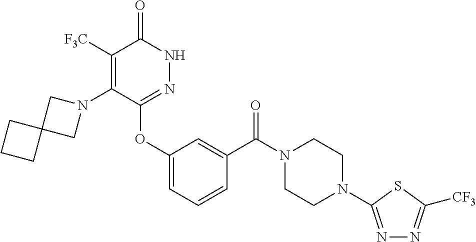 [SMILES: O=C(c1cccc(Oc2n[nH]c(=O)c(C(F)(F)F)c2N2CC3(CCC3)C2)c1)N1CCN(c2nnc(C(F)(F)F)s2)CC1]

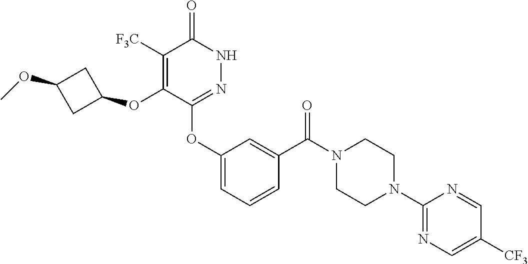 [SMILES: CO[C@H]1C[C@@H](Oc2c(Oc3cccc(C(=O)N4CCN(c5ncc(C(F)(F)F)cn5)CC4)c3)n[nH]c(=O)c2C(F)(F)F)C1]

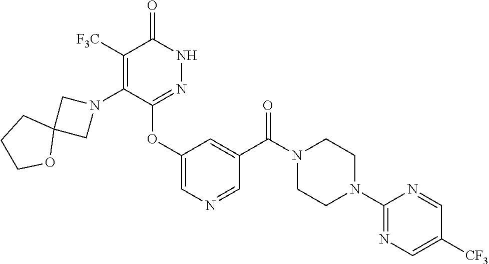 [SMILES: O=C(c1cncc(Oc2n[nH]c(=O)c(C(F)(F)F)c2N2CC3(CCCO3)C2)c1)N1CCN(c2ncc(C(F)(F)F)cn2)CC1]

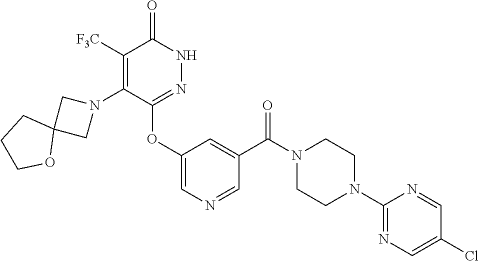 [SMILES: O=C(c1cncc(Oc2n[nH]c(=O)c(C(F)(F)F)c2N2CC3(CCCO3)C2)c1)N1CCN(c2ncc(Cl)cn2)CC1]

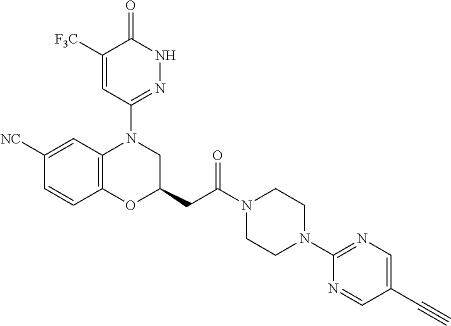 [SMILES: C#Cc1cnc(N2CCN(C(=O)C[C@@H]3CN(c4cc(C(F)(F)F)c(=O)[nH]n4)c4cc(C#N)ccc4O3)CC2)nc1]

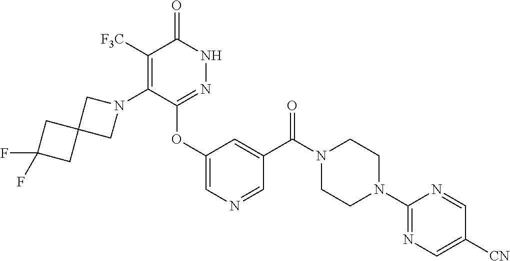 [SMILES: N#Cc1cnc(N2CCN(C(=O)c3cncc(Oc4n[nH]c(=O)c(C(F)(F)F)c4N4CC5(C4)CC(F)(F)C5)c3)CC2)nc1]

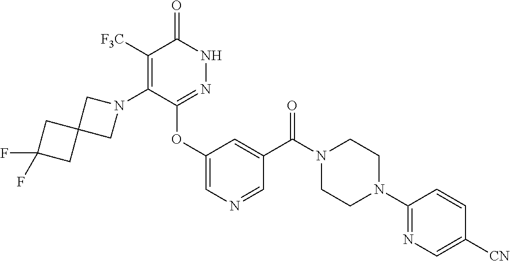 [SMILES: N#Cc1ccc(N2CCN(C(=O)c3cncc(Oc4n[nH]c(=O)c(C(F)(F)F)c4N4CC5(C4)CC(F)(F)C5)c3)CC2)nc1]

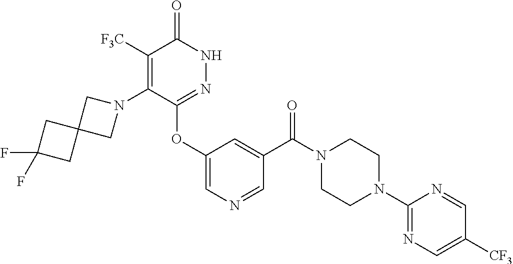 [SMILES: O=C(c1cncc(Oc2n[nH]c(=O)c(C(F)(F)F)c2N2CC3(C2)CC(F)(F)C3)c1)N1CCN(c2ncc(C(F)(F)F)cn2)CC1]

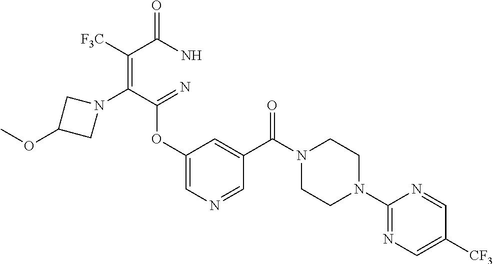 [SMILES: COC1CN(/C(C(=N)Oc2cncc(C(=O)N3CCN(c4ncc(C(F)(F)F)cn4)CC3)c2)=C(/C(N)=O)C(F)(F)F)C1]

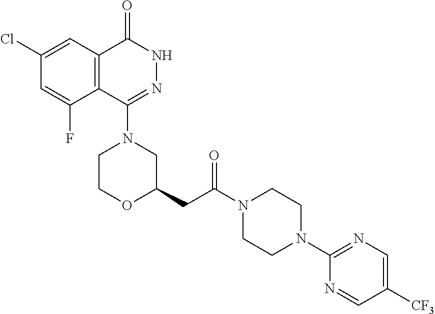 [SMILES: O=C(C[C@@H]1CN(c2n[nH]c(=O)c3cc(Cl)cc(F)c23)CCO1)N1CCN(c2ncc(C(F)(F)F)cn2)CC1]

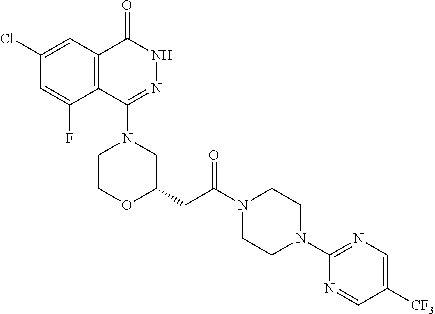 [SMILES: O=C(C[C@H]1CN(c2n[nH]c(=O)c3cc(Cl)cc(F)c23)CCO1)N1CCN(c2ncc(C(F)(F)F)cn2)CC1]

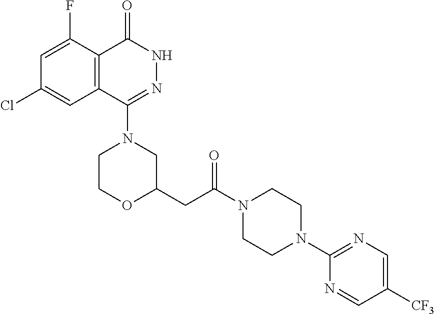 [SMILES: O=C(CC1CN(c2n[nH]c(=O)c3c(F)cc(Cl)cc23)CCO1)N1CCN(c2ncc(C(F)(F)F)cn2)CC1]

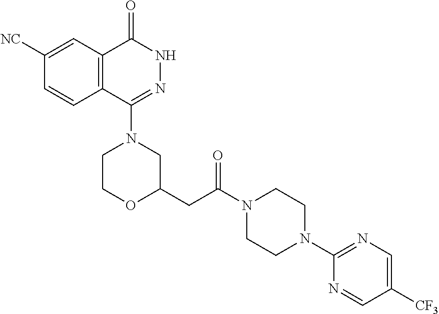 [SMILES: N#Cc1ccc2c(N3CCOC(CC(=O)N4CCN(c5ncc(C(F)(F)F)cn5)CC4)C3)n[nH]c(=O)c2c1]

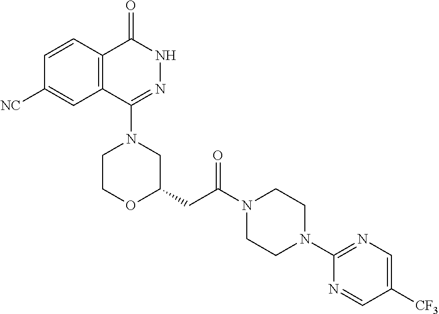 [SMILES: N#Cc1ccc2c(=O)[nH]nc(N3CCO[C@@H](CC(=O)N4CCN(c5ncc(C(F)(F)F)cn5)CC4)C3)c2c1]

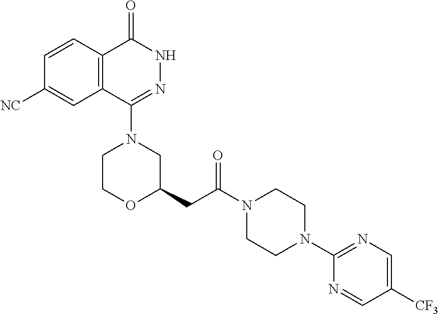 [SMILES: N#Cc1ccc2c(=O)[nH]nc(N3CCO[C@H](CC(=O)N4CCN(c5ncc(C(F)(F)F)cn5)CC4)C3)c2c1]

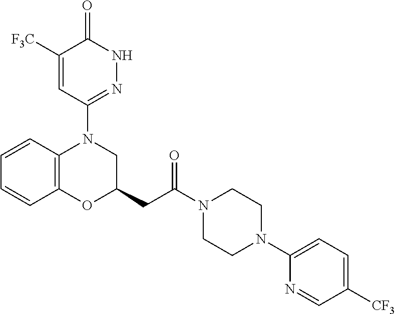 [SMILES: O=C(C[C@@H]1CN(c2cc(C(F)(F)F)c(=O)[nH]n2)c2ccccc2O1)N1CCN(c2ccc(C(F)(F)F)cn2)CC1]

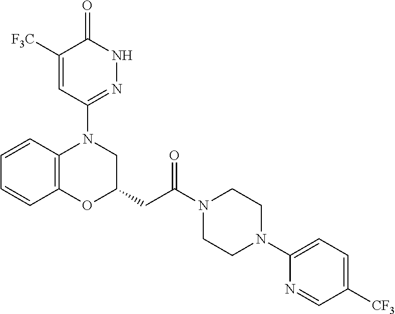 [SMILES: O=C(C[C@H]1CN(c2cc(C(F)(F)F)c(=O)[nH]n2)c2ccccc2O1)N1CCN(c2ccc(C(F)(F)F)cn2)CC1]

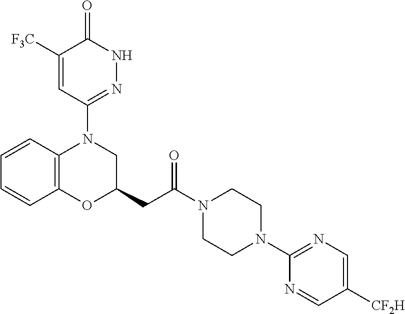 [SMILES: O=C(C[C@@H]1CN(c2cc(C(F)(F)F)c(=O)[nH]n2)c2ccccc2O1)N1CCN(c2ncc(C(F)F)cn2)CC1]

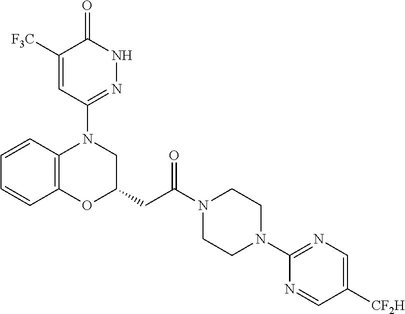 [SMILES: O=C(C[C@H]1CN(c2cc(C(F)(F)F)c(=O)[nH]n2)c2ccccc2O1)N1CCN(c2ncc(C(F)F)cn2)CC1]

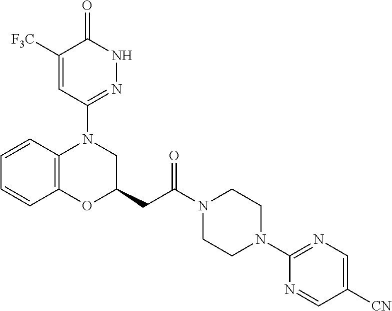 [SMILES: N#Cc1cnc(N2CCN(C(=O)C[C@@H]3CN(c4cc(C(F)(F)F)c(=O)[nH]n4)c4ccccc4O3)CC2)nc1]

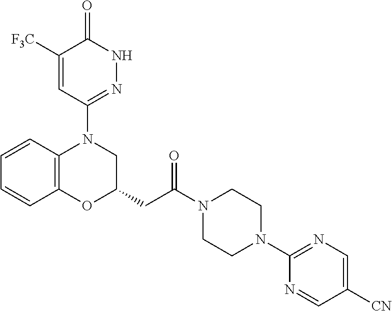 [SMILES: N#Cc1cnc(N2CCN(C(=O)C[C@H]3CN(c4cc(C(F)(F)F)c(=O)[nH]n4)c4ccccc4O3)CC2)nc1]

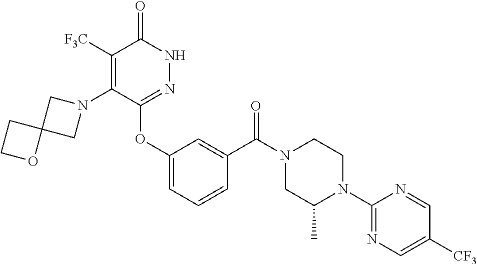 [SMILES: C[C@@H]1CN(C(=O)c2cccc(Oc3n[nH]c(=O)c(C(F)(F)F)c3N3CC4(CCO4)C3)c2)CCN1c1ncc(C(F)(F)F)cn1]

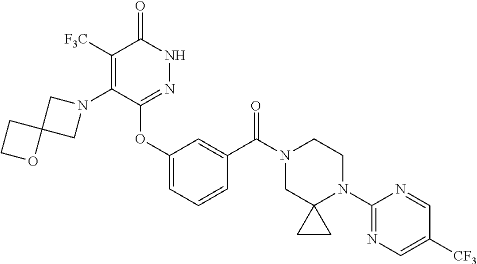 [SMILES: O=C(c1cccc(Oc2n[nH]c(=O)c(C(F)(F)F)c2N2CC3(CCO3)C2)c1)N1CCN(c2ncc(C(F)(F)F)cn2)C2(CC2)C1]

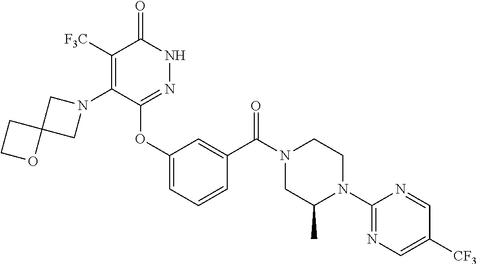 [SMILES: C[C@H]1CN(C(=O)c2cccc(Oc3n[nH]c(=O)c(C(F)(F)F)c3N3CC4(CCO4)C3)c2)CCN1c1ncc(C(F)(F)F)cn1]

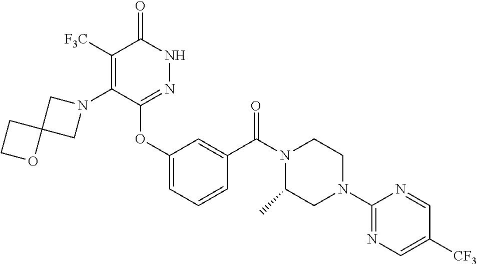 [SMILES: C[C@H]1CN(c2ncc(C(F)(F)F)cn2)CCN1C(=O)c1cccc(Oc2n[nH]c(=O)c(C(F)(F)F)c2N2CC3(CCO3)C2)c1]

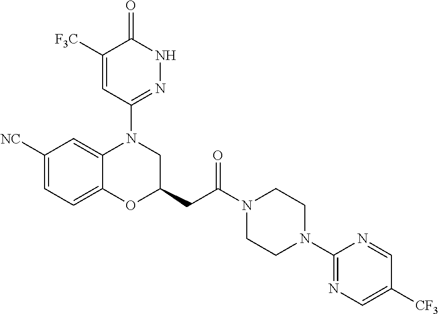 [SMILES: N#Cc1ccc2c(c1)N(c1cc(C(F)(F)F)c(=O)[nH]n1)C[C@@H](CC(=O)N1CCN(c3ncc(C(F)(F)F)cn3)CC1)O2]

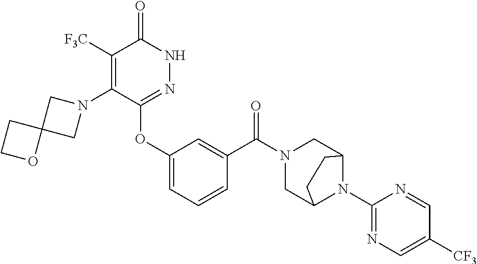 [SMILES: O=C(c1cccc(Oc2n[nH]c(=O)c(C(F)(F)F)c2N2CC3(CCO3)C2)c1)N1CC2CCC(C1)N2c1ncc(C(F)(F)F)cn1]

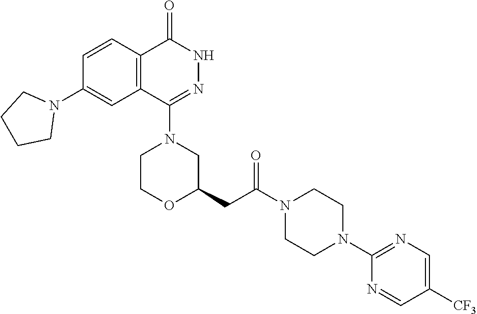 [SMILES: O=C(C[C@@H]1CN(c2n[nH]c(=O)c3ccc(N4CCCC4)cc23)CCO1)N1CCN(c2ncc(C(F)(F)F)cn2)CC1]

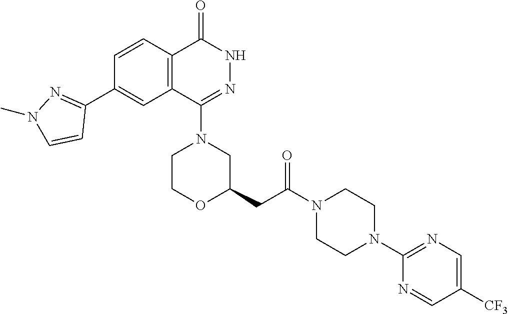 [SMILES: Cn1ccc(-c2ccc3c(=O)[nH]nc(N4CCO[C@H](CC(=O)N5CCN(c6ncc(C(F)(F)F)cn6)CC5)C4)c3c2)n1]

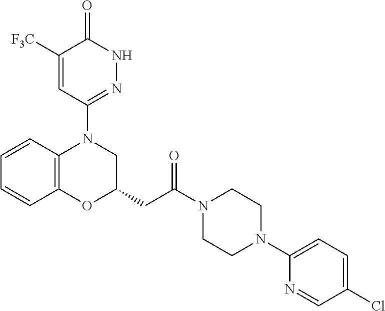 [SMILES: O=C(C[C@H]1CN(c2cc(C(F)(F)F)c(=O)[nH]n2)c2ccccc2O1)N1CCN(c2ccc(Cl)cn2)CC1]

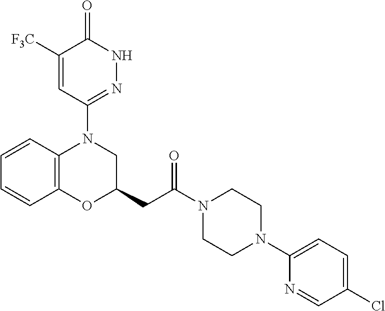 [SMILES: O=C(C[C@@H]1CN(c2cc(C(F)(F)F)c(=O)[nH]n2)c2ccccc2O1)N1CCN(c2ccc(Cl)cn2)CC1]

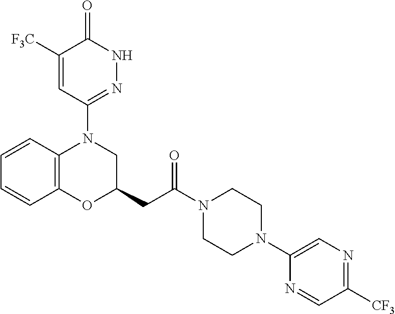 [SMILES: O=C(C[C@@H]1CN(c2cc(C(F)(F)F)c(=O)[nH]n2)c2ccccc2O1)N1CCN(c2cnc(C(F)(F)F)cn2)CC1]

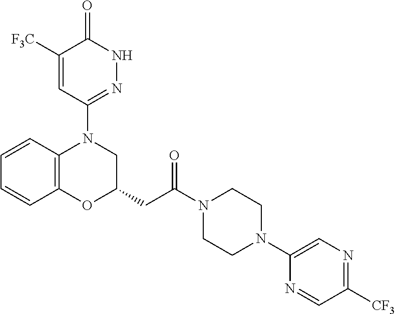 [SMILES: O=C(C[C@H]1CN(c2cc(C(F)(F)F)c(=O)[nH]n2)c2ccccc2O1)N1CCN(c2cnc(C(F)(F)F)cn2)CC1]

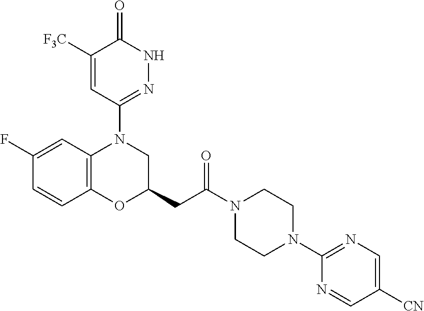 [SMILES: N#Cc1cnc(N2CCN(C(=O)C[C@@H]3CN(c4cc(C(F)(F)F)c(=O)[nH]n4)c4cc(F)ccc4O3)CC2)nc1]

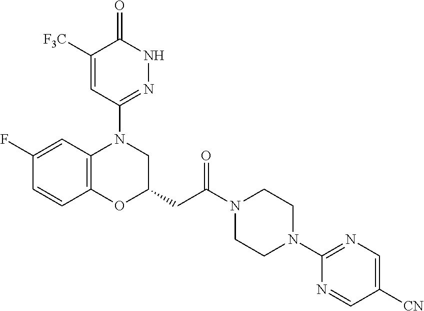 [SMILES: N#Cc1cnc(N2CCN(C(=O)C[C@H]3CN(c4cc(C(F)(F)F)c(=O)[nH]n4)c4cc(F)ccc4O3)CC2)nc1]

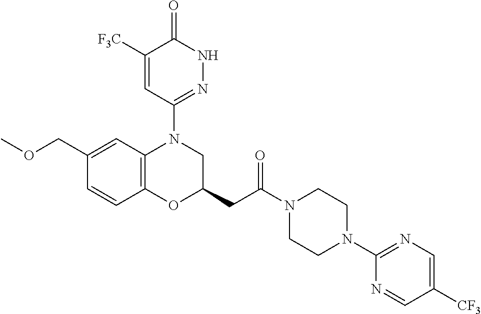 [SMILES: COCc1ccc2c(c1)N(c1cc(C(F)(F)F)c(=O)[nH]n1)C[C@@H](CC(=O)N1CCN(c3ncc(C(F)(F)F)cn3)CC1)O2]

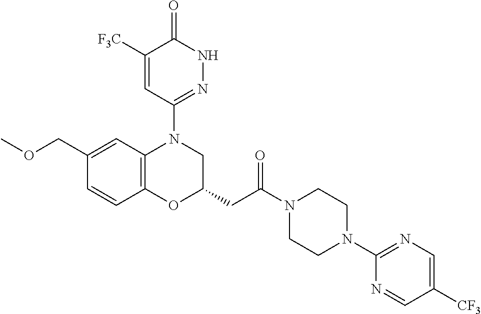 [SMILES: COCc1ccc2c(c1)N(c1cc(C(F)(F)F)c(=O)[nH]n1)C[C@H](CC(=O)N1CCN(c3ncc(C(F)(F)F)cn3)CC1)O2]

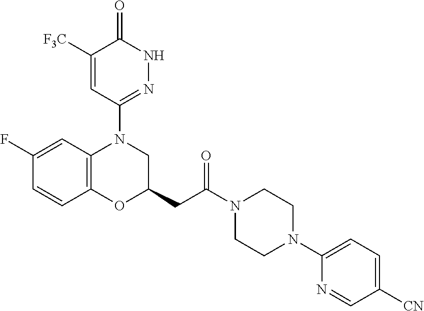 [SMILES: N#Cc1ccc(N2CCN(C(=O)C[C@@H]3CN(c4cc(C(F)(F)F)c(=O)[nH]n4)c4cc(F)ccc4O3)CC2)nc1]

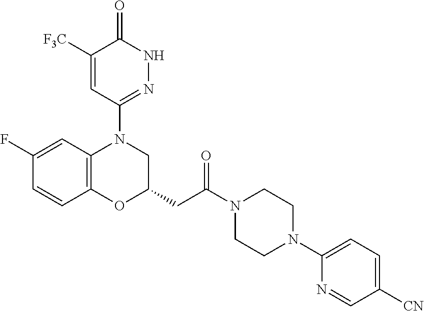 [SMILES: N#Cc1ccc(N2CCN(C(=O)C[C@H]3CN(c4cc(C(F)(F)F)c(=O)[nH]n4)c4cc(F)ccc4O3)CC2)nc1]

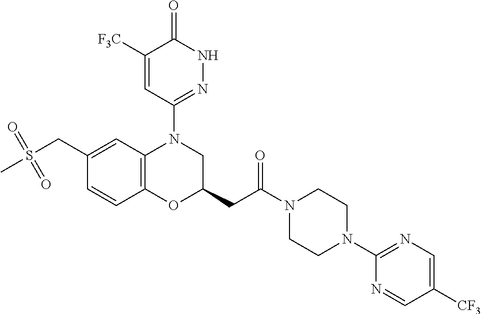 [SMILES: CS(=O)(=O)Cc1ccc2c(c1)N(c1cc(C(F)(F)F)c(=O)[nH]n1)C[C@@H](CC(=O)N1CCN(c3ncc(C(F)(F)F)cn3)CC1)O2]

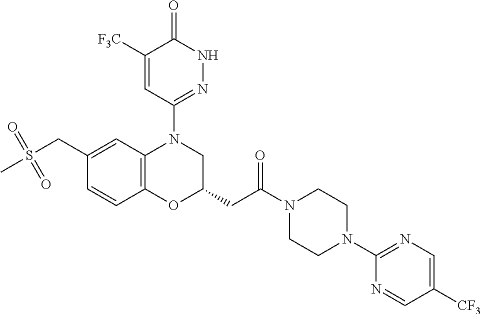 [SMILES: CS(=O)(=O)Cc1ccc2c(c1)N(c1cc(C(F)(F)F)c(=O)[nH]n1)C[C@H](CC(=O)N1CCN(c3ncc(C(F)(F)F)cn3)CC1)O2]

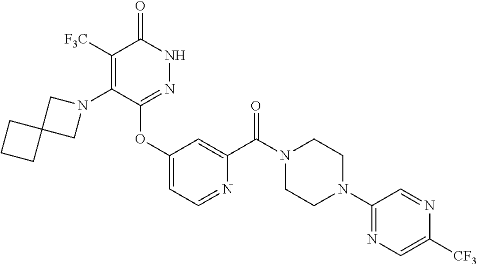 [SMILES: O=C(c1cc(Oc2n[nH]c(=O)c(C(F)(F)F)c2N2CC3(CCC3)C2)ccn1)N1CCN(c2cnc(C(F)(F)F)cn2)CC1]

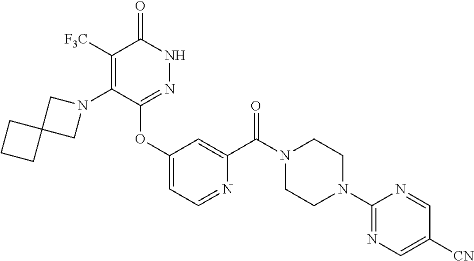 [SMILES: N#Cc1cnc(N2CCN(C(=O)c3cc(Oc4n[nH]c(=O)c(C(F)(F)F)c4N4CC5(CCC5)C4)ccn3)CC2)nc1]

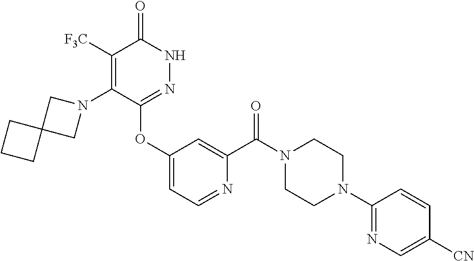 [SMILES: N#Cc1ccc(N2CCN(C(=O)c3cc(Oc4n[nH]c(=O)c(C(F)(F)F)c4N4CC5(CCC5)C4)ccn3)CC2)nc1]

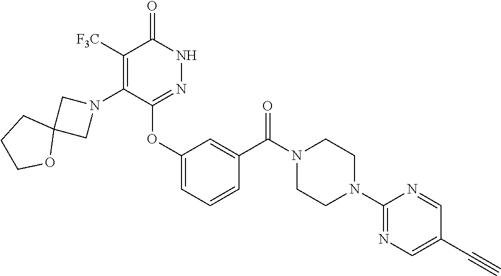 [SMILES: C#Cc1cnc(N2CCN(C(=O)c3cccc(Oc4n[nH]c(=O)c(C(F)(F)F)c4N4CC5(CCCO5)C4)c3)CC2)nc1]

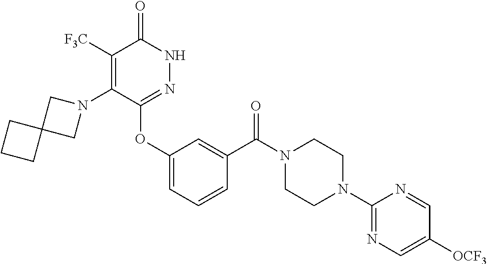 [SMILES: O=C(c1cccc(Oc2n[nH]c(=O)c(C(F)(F)F)c2N2CC3(CCC3)C2)c1)N1CCN(c2ncc(OC(F)(F)F)cn2)CC1]

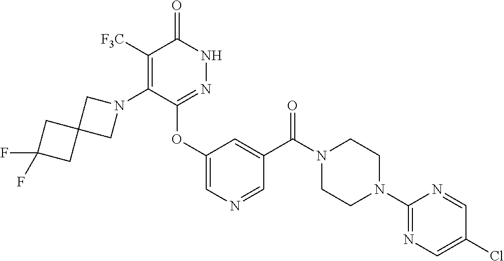 [SMILES: O=C(c1cncc(Oc2n[nH]c(=O)c(C(F)(F)F)c2N2CC3(C2)CC(F)(F)C3)c1)N1CCN(c2ncc(Cl)cn2)CC1]

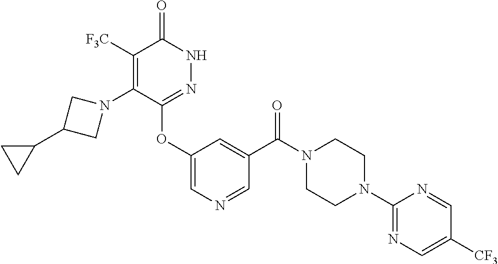 [SMILES: O=C(c1cncc(Oc2n[nH]c(=O)c(C(F)(F)F)c2N2CC(C3CC3)C2)c1)N1CCN(c2ncc(C(F)(F)F)cn2)CC1]

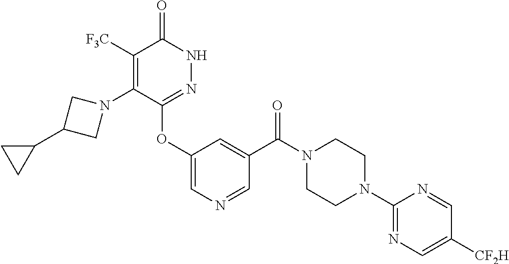 [SMILES: O=C(c1cncc(Oc2n[nH]c(=O)c(C(F)(F)F)c2N2CC(C3CC3)C2)c1)N1CCN(c2ncc(C(F)F)cn2)CC1]

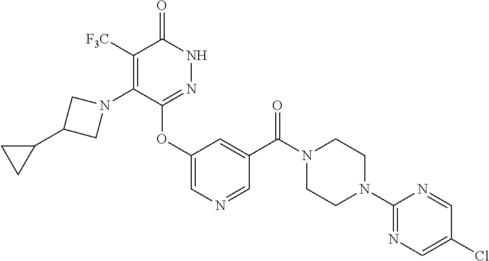 [SMILES: O=C(c1cncc(Oc2n[nH]c(=O)c(C(F)(F)F)c2N2CC(C3CC3)C2)c1)N1CCN(c2ncc(Cl)cn2)CC1]

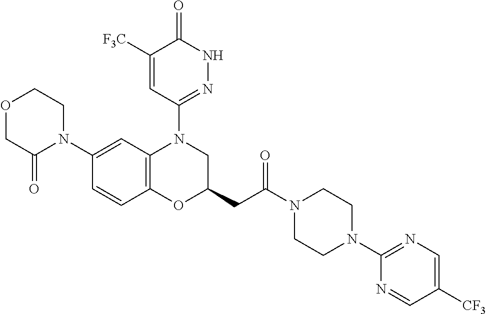 [SMILES: O=C(C[C@@H]1CN(c2cc(C(F)(F)F)c(=O)[nH]n2)c2cc(N3CCOCC3=O)ccc2O1)N1CCN(c2ncc(C(F)(F)F)cn2)CC1]

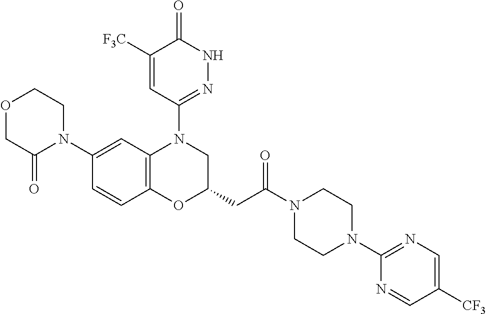 [SMILES: O=C(C[C@H]1CN(c2cc(C(F)(F)F)c(=O)[nH]n2)c2cc(N3CCOCC3=O)ccc2O1)N1CCN(c2ncc(C(F)(F)F)cn2)CC1]

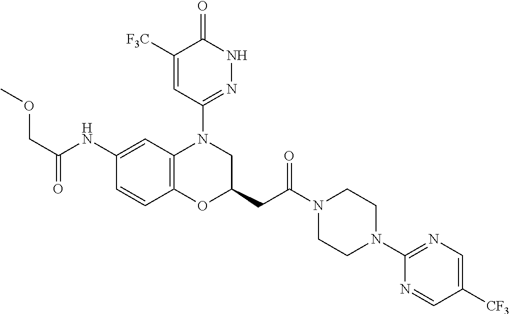 [SMILES: COCC(=O)Nc1ccc2c(c1)N(c1cc(C(F)(F)F)c(=O)[nH]n1)C[C@@H](CC(=O)N1CCN(c3ncc(C(F)(F)F)cn3)CC1)O2]

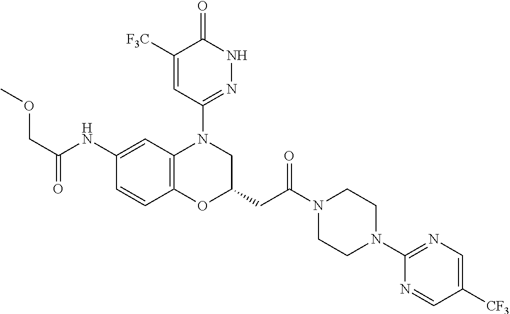 [SMILES: COCC(=O)Nc1ccc2c(c1)N(c1cc(C(F)(F)F)c(=O)[nH]n1)C[C@H](CC(=O)N1CCN(c3ncc(C(F)(F)F)cn3)CC1)O2]

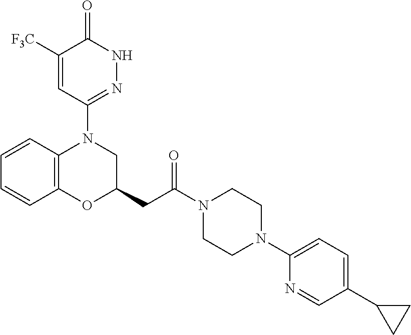 [SMILES: O=C(C[C@@H]1CN(c2cc(C(F)(F)F)c(=O)[nH]n2)c2ccccc2O1)N1CCN(c2ccc(C3CC3)cn2)CC1]

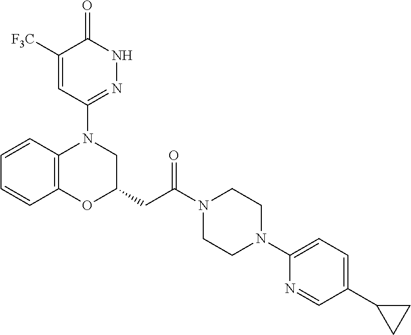 [SMILES: O=C(C[C@H]1CN(c2cc(C(F)(F)F)c(=O)[nH]n2)c2ccccc2O1)N1CCN(c2ccc(C3CC3)cn2)CC1]

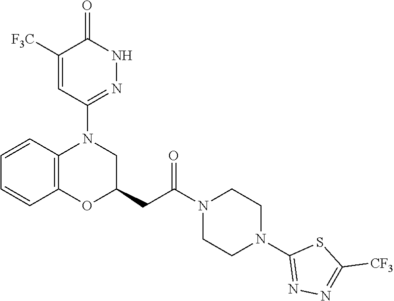 [SMILES: O=C(C[C@@H]1CN(c2cc(C(F)(F)F)c(=O)[nH]n2)c2ccccc2O1)N1CCN(c2nnc(C(F)(F)F)s2)CC1]

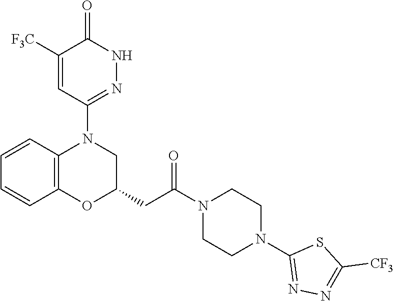 [SMILES: O=C(C[C@H]1CN(c2cc(C(F)(F)F)c(=O)[nH]n2)c2ccccc2O1)N1CCN(c2nnc(C(F)(F)F)s2)CC1]

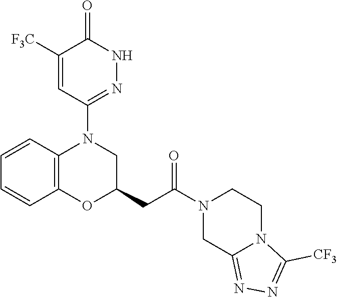 [SMILES: O=C(C[C@@H]1CN(c2cc(C(F)(F)F)c(=O)[nH]n2)c2ccccc2O1)N1CCn2c(nnc2C(F)(F)F)C1]

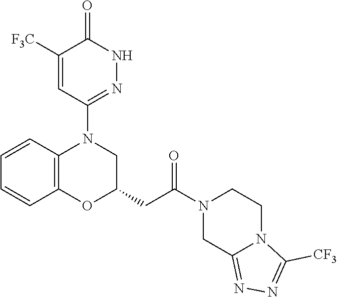 [SMILES: O=C(C[C@H]1CN(c2cc(C(F)(F)F)c(=O)[nH]n2)c2ccccc2O1)N1CCn2c(nnc2C(F)(F)F)C1]

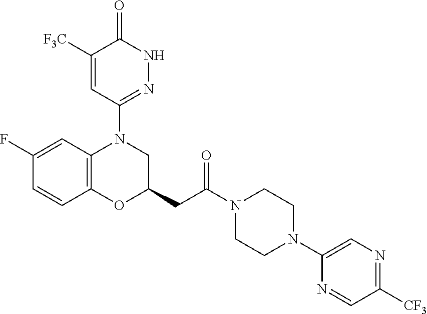 [SMILES: O=C(C[C@@H]1CN(c2cc(C(F)(F)F)c(=O)[nH]n2)c2cc(F)ccc2O1)N1CCN(c2cnc(C(F)(F)F)cn2)CC1]

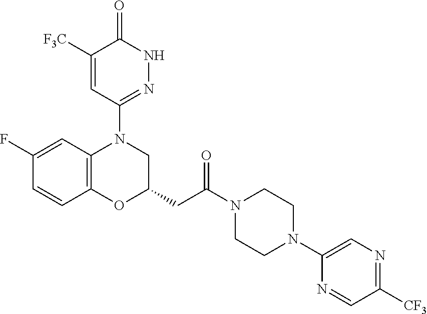 [SMILES: O=C(C[C@H]1CN(c2cc(C(F)(F)F)c(=O)[nH]n2)c2cc(F)ccc2O1)N1CCN(c2cnc(C(F)(F)F)cn2)CC1]

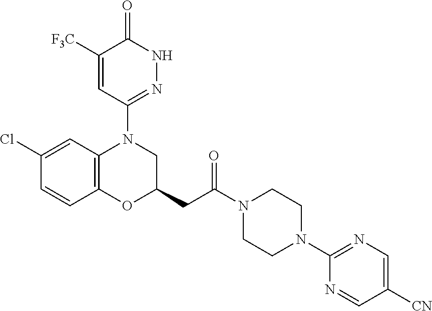 [SMILES: N#Cc1cnc(N2CCN(C(=O)C[C@@H]3CN(c4cc(C(F)(F)F)c(=O)[nH]n4)c4cc(Cl)ccc4O3)CC2)nc1]

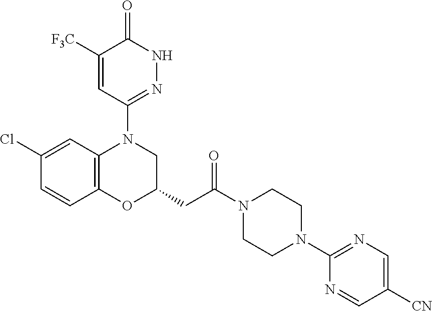 [SMILES: N#Cc1cnc(N2CCN(C(=O)C[C@H]3CN(c4cc(C(F)(F)F)c(=O)[nH]n4)c4cc(Cl)ccc4O3)CC2)nc1]

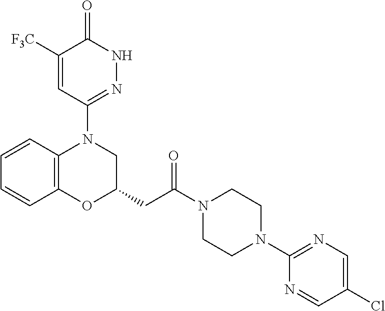 [SMILES: O=C(C[C@H]1CN(c2cc(C(F)(F)F)c(=O)[nH]n2)c2ccccc2O1)N1CCN(c2ncc(Cl)cn2)CC1]

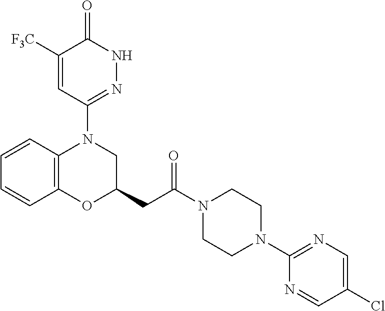 [SMILES: O=C(C[C@@H]1CN(c2cc(C(F)(F)F)c(=O)[nH]n2)c2ccccc2O1)N1CCN(c2ncc(Cl)cn2)CC1]

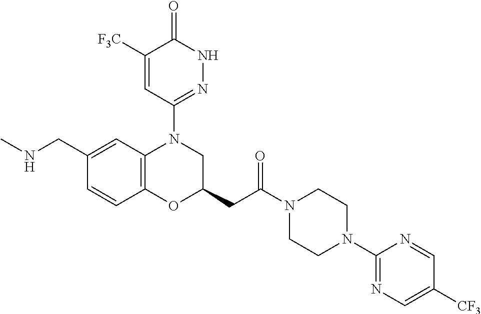 [SMILES: CNCc1ccc2c(c1)N(c1cc(C(F)(F)F)c(=O)[nH]n1)C[C@@H](CC(=O)N1CCN(c3ncc(C(F)(F)F)cn3)CC1)O2]

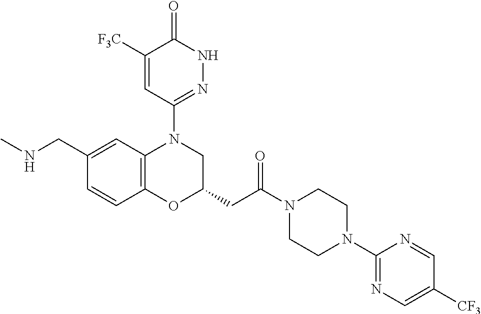 [SMILES: CNCc1ccc2c(c1)N(c1cc(C(F)(F)F)c(=O)[nH]n1)C[C@H](CC(=O)N1CCN(c3ncc(C(F)(F)F)cn3)CC1)O2]

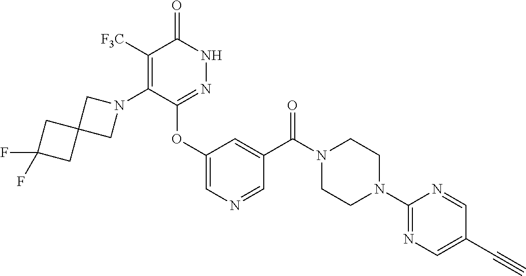 [SMILES: C#Cc1cnc(N2CCN(C(=O)c3cncc(Oc4n[nH]c(=O)c(C(F)(F)F)c4N4CC5(C4)CC(F)(F)C5)c3)CC2)nc1]

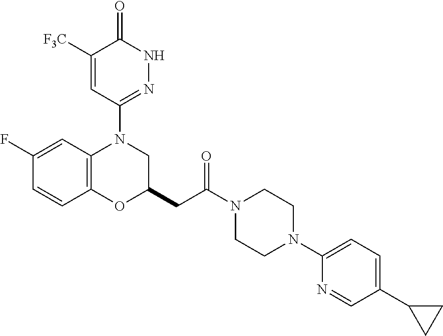

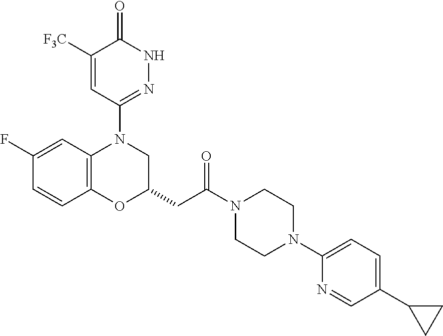

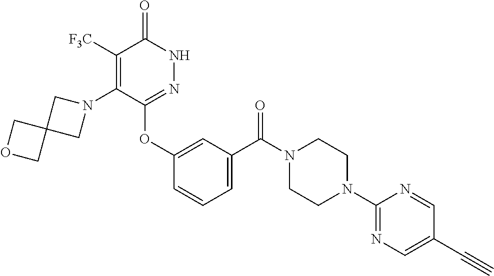 [SMILES: C#Cc1cnc(N2CCN(C(=O)c3cccc(Oc4n[nH]c(=O)c(C(F)(F)F)c4N4CC5(COC5)C4)c3)CC2)nc1]

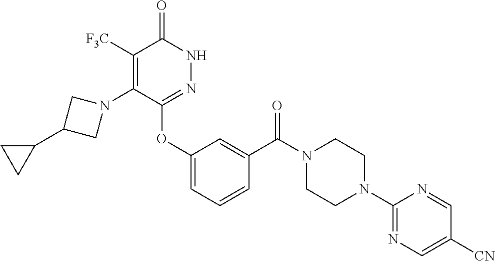 [SMILES: N#Cc1cnc(N2CCN(C(=O)c3cccc(Oc4n[nH]c(=O)c(C(F)(F)F)c4N4CC(C5CC5)C4)c3)CC2)nc1]

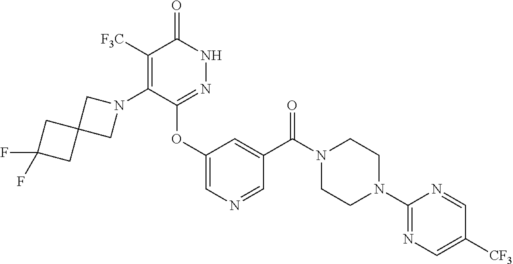 [SMILES: O=C(c1cncc(Oc2n[nH]c(=O)c(C(F)(F)F)c2N2CC3(C2)CC(F)(F)C3)c1)N1CCN(c2ncc(C(F)(F)F)cn2)CC1]

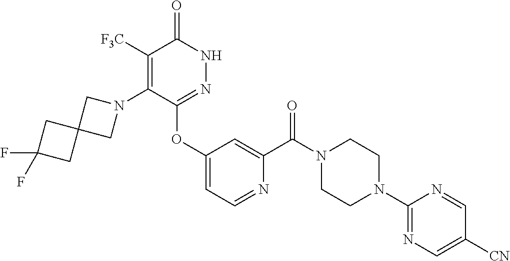 [SMILES: N#Cc1cnc(N2CCN(C(=O)c3cc(Oc4n[nH]c(=O)c(C(F)(F)F)c4N4CC5(C4)CC(F)(F)C5)ccn3)CC2)nc1]

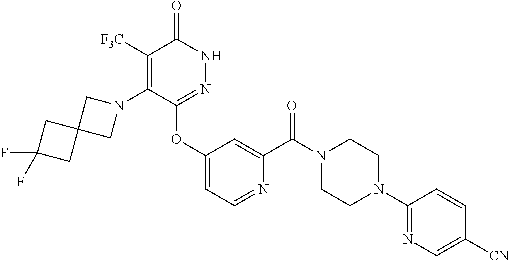 [SMILES: N#Cc1ccc(N2CCN(C(=O)c3cc(Oc4n[nH]c(=O)c(C(F)(F)F)c4N4CC5(C4)CC(F)(F)C5)ccn3)CC2)nc1]

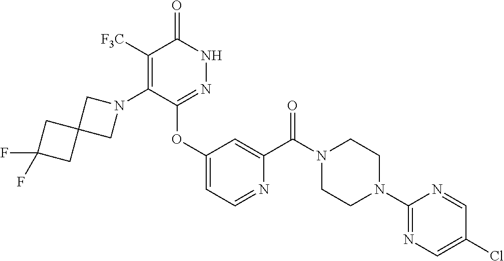 [SMILES: O=C(c1cc(Oc2n[nH]c(=O)c(C(F)(F)F)c2N2CC3(C2)CC(F)(F)C3)ccn1)N1CCN(c2ncc(Cl)cn2)CC1]

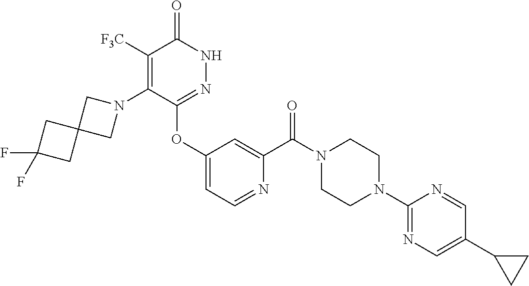 [SMILES: O=C(c1cc(Oc2n[nH]c(=O)c(C(F)(F)F)c2N2CC3(C2)CC(F)(F)C3)ccn1)N1CCN(c2ncc(C3CC3)cn2)CC1]

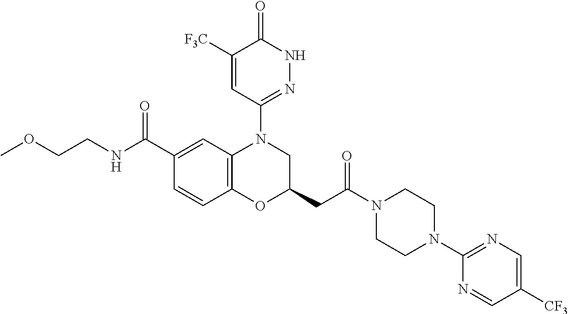 [SMILES: COCCNC(=O)c1ccc2c(c1)N(c1cc(C(F)(F)F)c(=O)[nH]n1)C[C@@H](CC(=O)N1CCN(c3ncc(C(F)(F)F)cn3)CC1)O2]

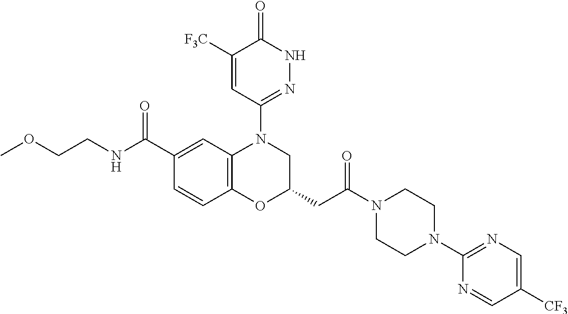 [SMILES: COCCNC(=O)c1ccc2c(c1)N(c1cc(C(F)(F)F)c(=O)[nH]n1)C[C@H](CC(=O)N1CCN(c3ncc(C(F)(F)F)cn3)CC1)O2]

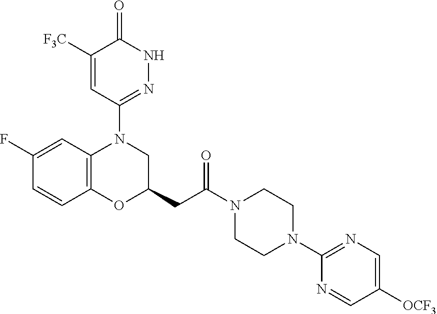 [SMILES: O=C(C[C@@H]1CN(c2cc(C(F)(F)F)c(=O)[nH]n2)c2cc(F)ccc2O1)N1CCN(c2ncc(OC(F)(F)F)cn2)CC1]

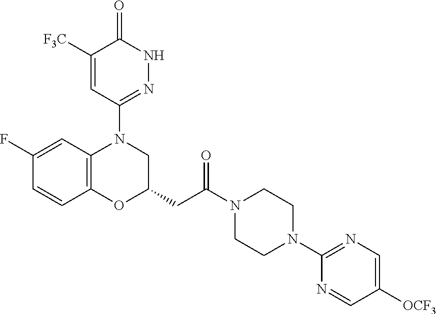 [SMILES: O=C(C[C@H]1CN(c2cc(C(F)(F)F)c(=O)[nH]n2)c2cc(F)ccc2O1)N1CCN(c2ncc(OC(F)(F)F)cn2)CC1]

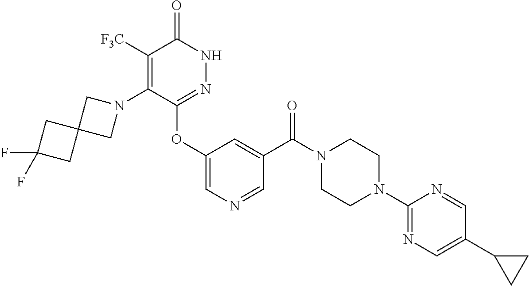 [SMILES: O=C(c1cncc(Oc2n[nH]c(=O)c(C(F)(F)F)c2N2CC3(C2)CC(F)(F)C3)c1)N1CCN(c2ncc(C3CC3)cn2)CC1]

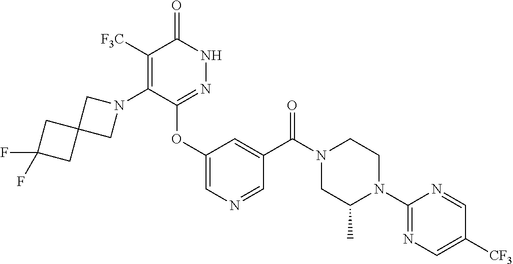 [SMILES: C[C@@H]1CN(C(=O)c2cncc(Oc3n[nH]c(=O)c(C(F)(F)F)c3N3CC4(C3)CC(F)(F)C4)c2)CCN1c1ncc(C(F)(F)F)cn1]

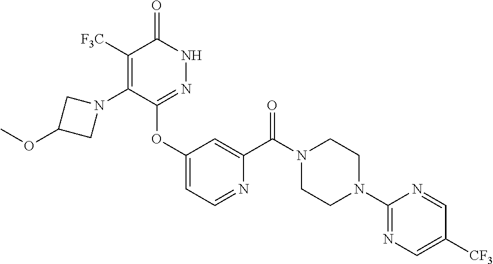 [SMILES: COC1CN(c2c(Oc3ccnc(C(=O)N4CCN(c5ncc(C(F)(F)F)cn5)CC4)c3)n[nH]c(=O)c2C(F)(F)F)C1]

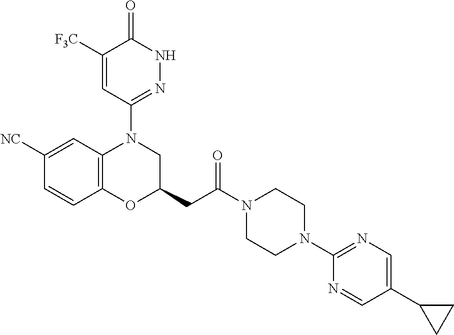 [SMILES: N#Cc1ccc2c(c1)N(c1cc(C(F)(F)F)c(=O)[nH]n1)C[C@@H](CC(=O)N1CCN(c3ncc(C4CC4)cn3)CC1)O2]

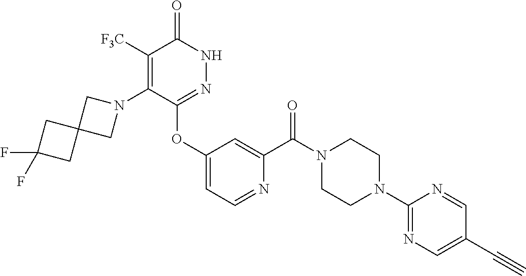 [SMILES: C#Cc1cnc(N2CCN(C(=O)c3cc(Oc4n[nH]c(=O)c(C(F)(F)F)c4N4CC5(C4)CC(F)(F)C5)ccn3)CC2)nc1]

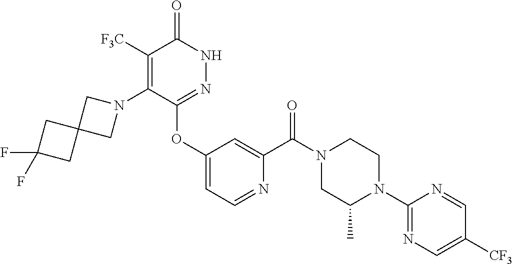 [SMILES: C[C@@H]1CN(C(=O)c2cc(Oc3n[nH]c(=O)c(C(F)(F)F)c3N3CC4(C3)CC(F)(F)C4)ccn2)CCN1c1ncc(C(F)(F)F)cn1]

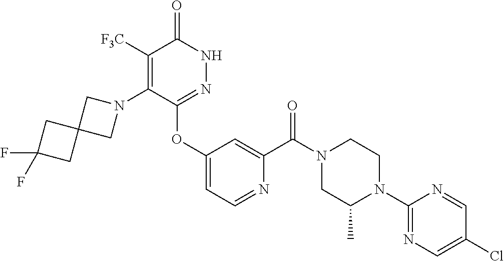 [SMILES: C[C@@H]1CN(C(=O)c2cc(Oc3n[nH]c(=O)c(C(F)(F)F)c3N3CC4(C3)CC(F)(F)C4)ccn2)CCN1c1ncc(Cl)cn1]

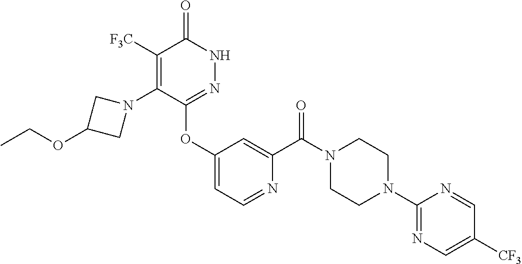 [SMILES: CCOC1CN(c2c(Oc3ccnc(C(=O)N4CCN(c5ncc(C(F)(F)F)cn5)CC4)c3)n[nH]c(=O)c2C(F)(F)F)C1]

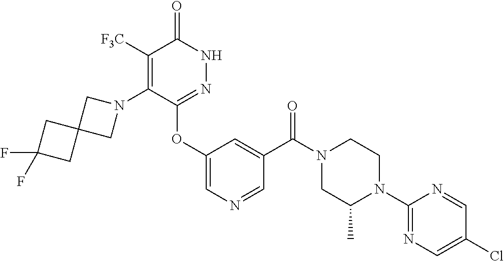 [SMILES: C[C@@H]1CN(C(=O)c2cncc(Oc3n[nH]c(=O)c(C(F)(F)F)c3N3CC4(C3)CC(F)(F)C4)c2)CCN1c1ncc(Cl)cn1]

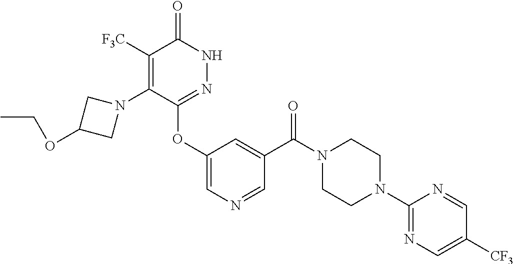 [SMILES: CCOC1CN(c2c(Oc3cncc(C(=O)N4CCN(c5ncc(C(F)(F)F)cn5)CC4)c3)n[nH]c(=O)c2C(F)(F)F)C1]

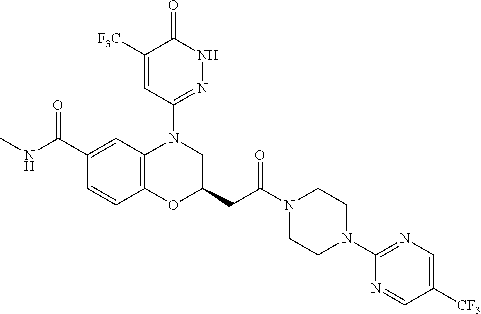 [SMILES: CNC(=O)c1ccc2c(c1)N(c1cc(C(F)(F)F)c(=O)[nH]n1)C[C@@H](CC(=O)N1CCN(c3ncc(C(F)(F)F)cn3)CC1)O2]

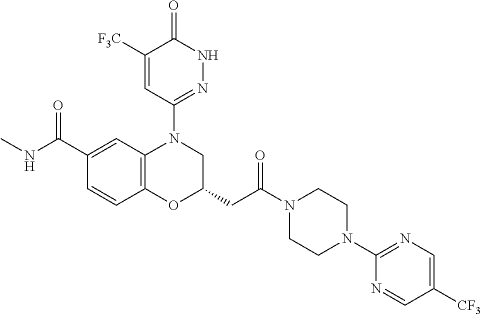 [SMILES: CNC(=O)c1ccc2c(c1)N(c1cc(C(F)(F)F)c(=O)[nH]n1)C[C@H](CC(=O)N1CCN(c3ncc(C(F)(F)F)cn3)CC1)O2]

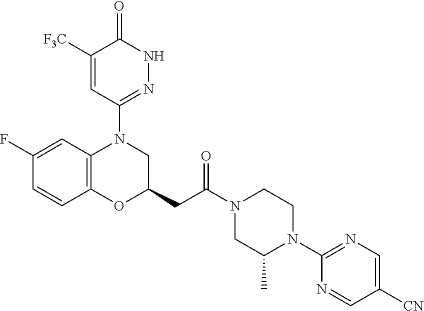 [SMILES: C[C@@H]1CN(C(=O)C[C@@H]2CN(c3cc(C(F)(F)F)c(=O)[nH]n3)c3cc(F)ccc3O2)CCN1c1ncc(C#N)cn1]

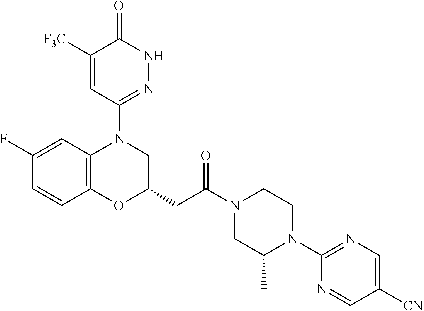 [SMILES: C[C@@H]1CN(C(=O)C[C@H]2CN(c3cc(C(F)(F)F)c(=O)[nH]n3)c3cc(F)ccc3O2)CCN1c1ncc(C#N)cn1]

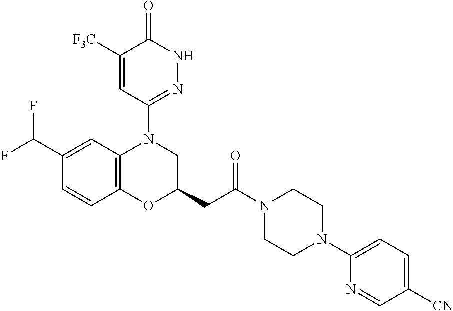 [SMILES: N#Cc1ccc(N2CCN(C(=O)C[C@@H]3CN(c4cc(C(F)(F)F)c(=O)[nH]n4)c4cc(C(F)F)ccc4O3)CC2)nc1]

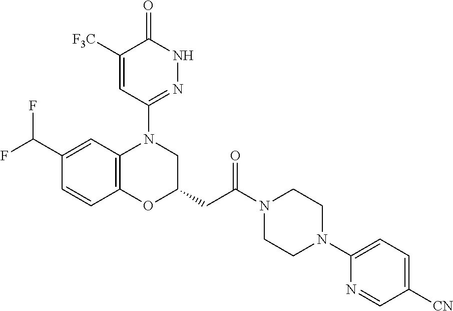 [SMILES: N#Cc1ccc(N2CCN(C(=O)C[C@H]3CN(c4cc(C(F)(F)F)c(=O)[nH]n4)c4cc(C(F)F)ccc4O3)CC2)nc1]

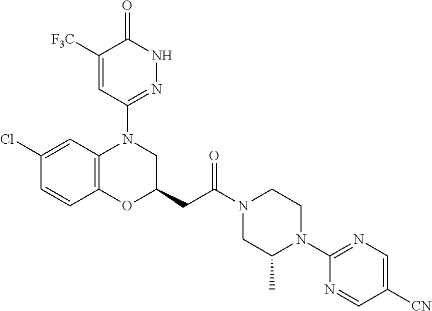 [SMILES: C[C@@H]1CN(C(=O)C[C@@H]2CN(c3cc(C(F)(F)F)c(=O)[nH]n3)c3cc(Cl)ccc3O2)CCN1c1ncc(C#N)cn1]

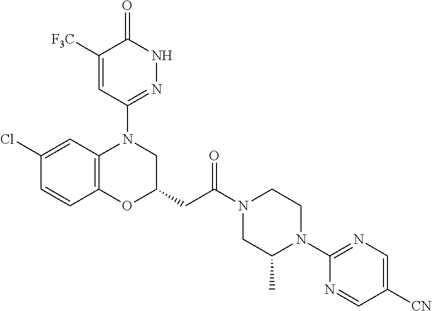 [SMILES: C[C@@H]1CN(C(=O)C[C@H]2CN(c3cc(C(F)(F)F)c(=O)[nH]n3)c3cc(Cl)ccc3O2)CCN1c1ncc(C#N)cn1]

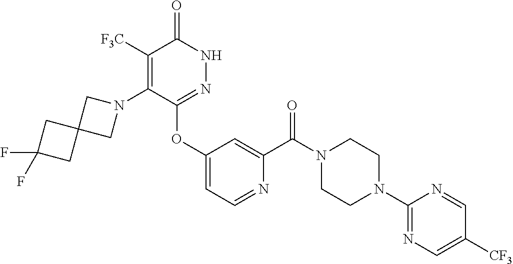 [SMILES: O=C(c1cc(Oc2n[nH]c(=O)c(C(F)(F)F)c2N2CC3(C2)CC(F)(F)C3)ccn1)N1CCN(c2ncc(C(F)(F)F)cn2)CC1]

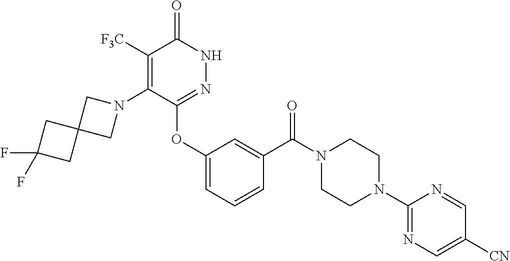 [SMILES: N#Cc1cnc(N2CCN(C(=O)c3cccc(Oc4n[nH]c(=O)c(C(F)(F)F)c4N4CC5(C4)CC(F)(F)C5)c3)CC2)nc1]

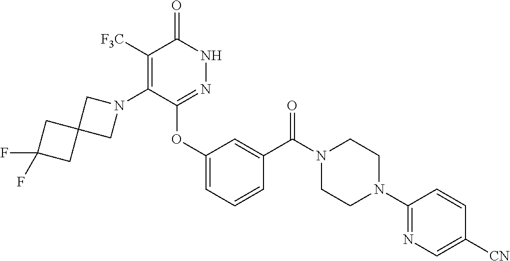 [SMILES: N#Cc1ccc(N2CCN(C(=O)c3cccc(Oc4n[nH]c(=O)c(C(F)(F)F)c4N4CC5(C4)CC(F)(F)C5)c3)CC2)nc1]

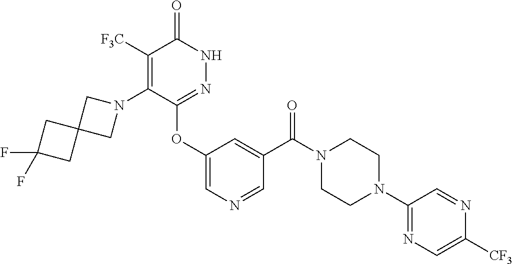 [SMILES: O=C(c1cncc(Oc2n[nH]c(=O)c(C(F)(F)F)c2N2CC3(C2)CC(F)(F)C3)c1)N1CCN(c2cnc(C(F)(F)F)cn2)CC1]

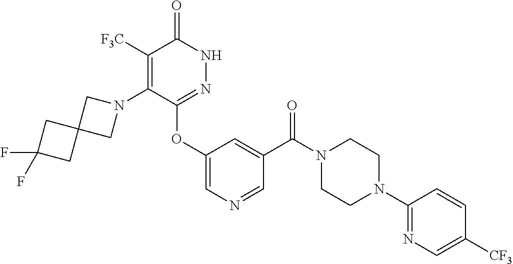 [SMILES: O=C(c1cncc(Oc2n[nH]c(=O)c(C(F)(F)F)c2N2CC3(C2)CC(F)(F)C3)c1)N1CCN(c2ccc(C(F)(F)F)cn2)CC1]

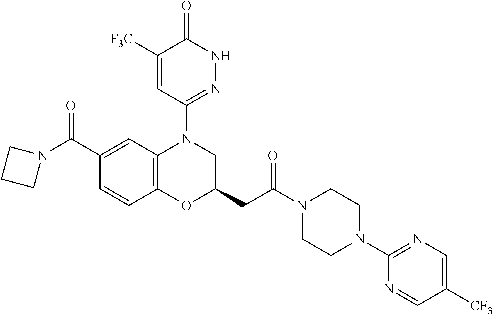 [SMILES: O=C(C[C@@H]1CN(c2cc(C(F)(F)F)c(=O)[nH]n2)c2cc(C(=O)N3CCC3)ccc2O1)N1CCN(c2ncc(C(F)(F)F)cn2)CC1]

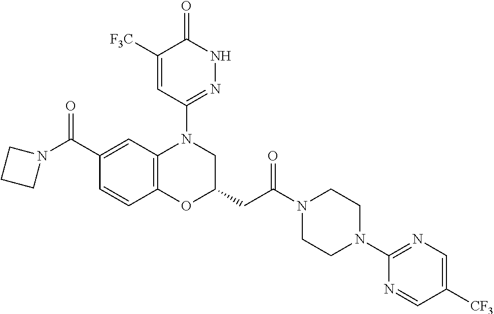 [SMILES: O=C(C[C@H]1CN(c2cc(C(F)(F)F)c(=O)[nH]n2)c2cc(C(=O)N3CCC3)ccc2O1)N1CCN(c2ncc(C(F)(F)F)cn2)CC1]

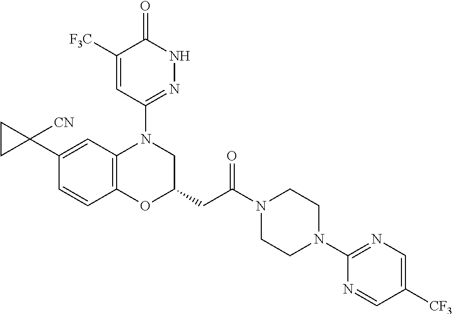 [SMILES: N#CC1(c2ccc3c(c2)N(c2cc(C(F)(F)F)c(=O)[nH]n2)C[C@H](CC(=O)N2CCN(c4ncc(C(F)(F)F)cn4)CC2)O3)CC1]

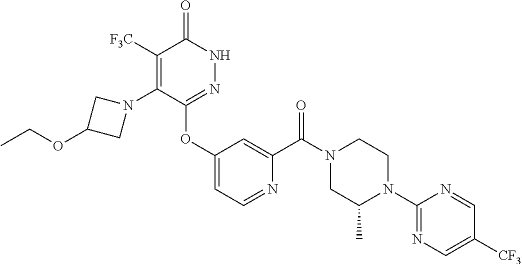 [SMILES: CCOC1CN(c2c(Oc3ccnc(C(=O)N4CCN(c5ncc(C(F)(F)F)cn5)[C@H](C)C4)c3)n[nH]c(=O)c2C(F)(F)F)C1]

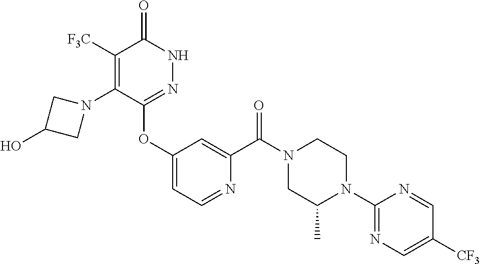 [SMILES: C[C@@H]1CN(C(=O)c2cc(Oc3n[nH]c(=O)c(C(F)(F)F)c3N3CC(O)C3)ccn2)CCN1c1ncc(C(F)(F)F)cn1]

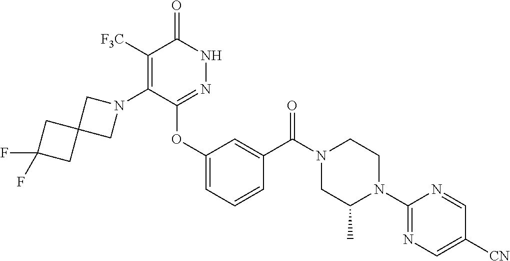 [SMILES: C[C@@H]1CN(C(=O)c2cccc(Oc3n[nH]c(=O)c(C(F)(F)F)c3N3CC4(C3)CC(F)(F)C4)c2)CCN1c1ncc(C#N)cn1]

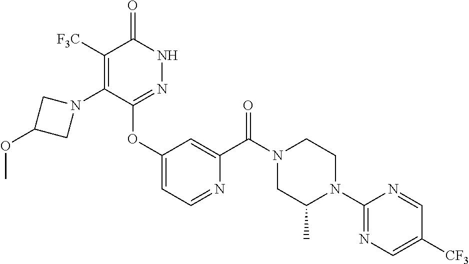 [SMILES: COC1CN(c2c(Oc3ccnc(C(=O)N4CCN(c5ncc(C(F)(F)F)cn5)[C@H](C)C4)c3)n[nH]c(=O)c2C(F)(F)F)C1]

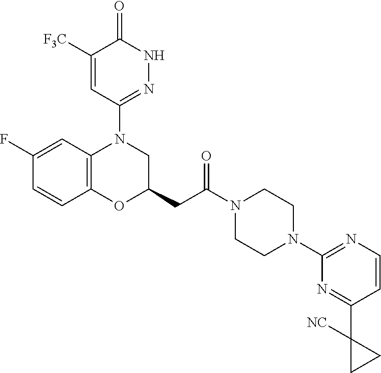 [SMILES: N#CC1(c2ccnc(N3CCN(C(=O)C[C@@H]4CN(c5cc(C(F)(F)F)c(=O)[nH]n5)c5cc(F)ccc5O4)CC3)n2)CC1]

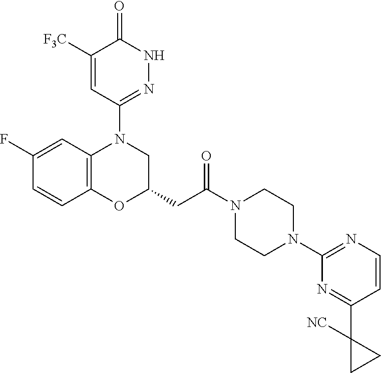 [SMILES: N#CC1(c2ccnc(N3CCN(C(=O)C[C@H]4CN(c5cc(C(F)(F)F)c(=O)[nH]n5)c5cc(F)ccc5O4)CC3)n2)CC1]

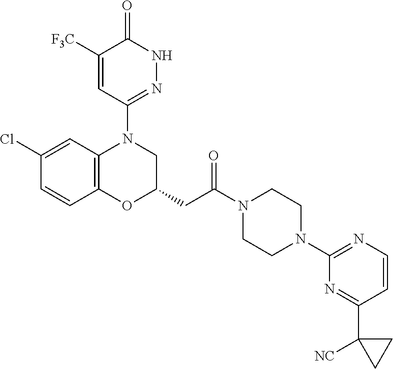 [SMILES: N#CC1(c2ccnc(N3CCN(C(=O)C[C@H]4CN(c5cc(C(F)(F)F)c(=O)[nH]n5)c5cc(Cl)ccc5O4)CC3)n2)CC1]

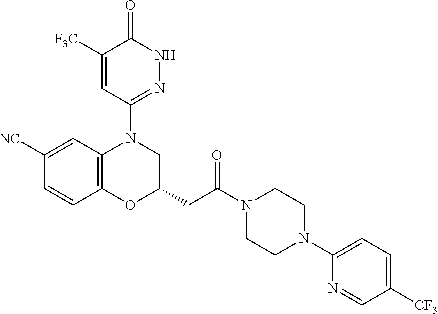 [SMILES: N#Cc1ccc2c(c1)N(c1cc(C(F)(F)F)c(=O)[nH]n1)C[C@H](CC(=O)N1CCN(c3ccc(C(F)(F)F)cn3)CC1)O2]

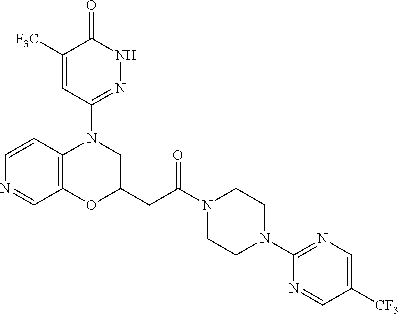 [SMILES: O=C(CC1CN(c2cc(C(F)(F)F)c(=O)[nH]n2)c2ccncc2O1)N1CCN(c2ncc(C(F)(F)F)cn2)CC1]

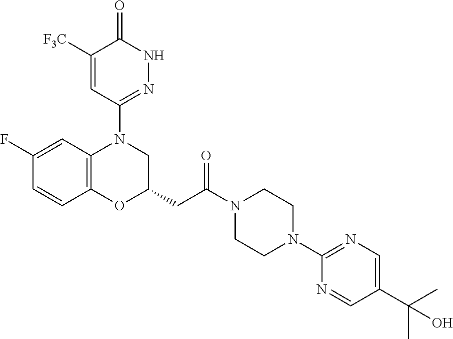 [SMILES: CC(C)(O)c1cnc(N2CCN(C(=O)C[C@H]3CN(c4cc(C(F)(F)F)c(=O)[nH]n4)c4cc(F)ccc4O3)CC2)nc1]

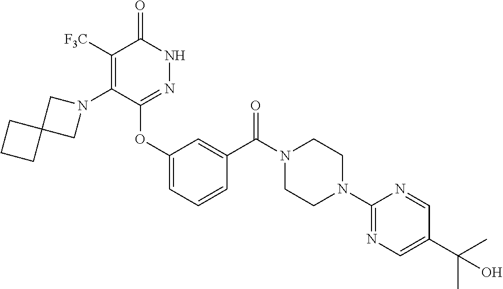 [SMILES: CC(C)(O)c1cnc(N2CCN(C(=O)c3cccc(Oc4n[nH]c(=O)c(C(F)(F)F)c4N4CC5(CCC5)C4)c3)CC2)nc1]

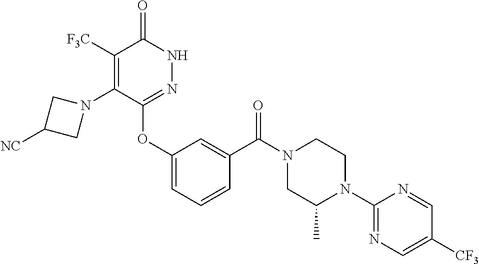 [SMILES: C[C@@H]1CN(C(=O)c2cccc(Oc3n[nH]c(=O)c(C(F)(F)F)c3N3CC(C#N)C3)c2)CCN1c1ncc(C(F)(F)F)cn1]

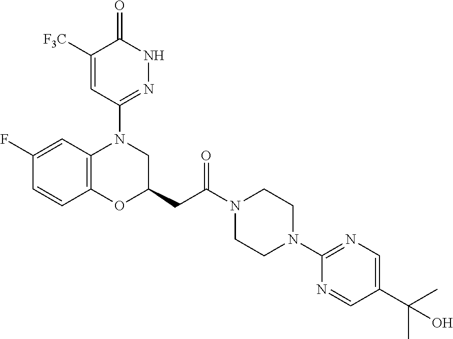 [SMILES: CC(C)(O)c1cnc(N2CCN(C(=O)C[C@@H]3CN(c4cc(C(F)(F)F)c(=O)[nH]n4)c4cc(F)ccc4O3)CC2)nc1]

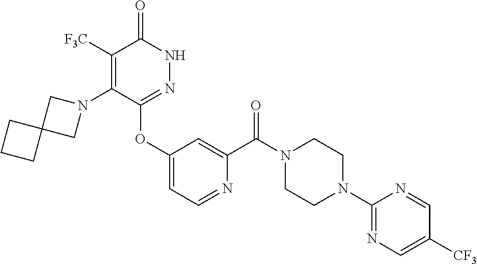 [SMILES: O=C(c1cc(Oc2n[nH]c(=O)c(C(F)(F)F)c2N2CC3(CCC3)C2)ccn1)N1CCN(c2ncc(C(F)(F)F)cn2)CC1]

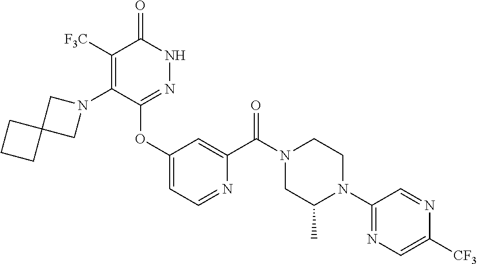 [SMILES: C[C@@H]1CN(C(=O)c2cc(Oc3n[nH]c(=O)c(C(F)(F)F)c3N3CC4(CCC4)C3)ccn2)CCN1c1cnc(C(F)(F)F)cn1]

In some embodiments, when applicable, a compound shown Table A can have an enantiomeric excess (“ee”) of greater than 60%, such as having greater than 80% ee, greater than 90% ee, greater than 90% ee, greater than 95% ee, greater than 98% ee, greater than 99% ee, or with the other enantiomer in a non-detectable amount. In some embodiments, when applicable, a compound shown Table A can also exist as a mixture of stereoisomers in any ratio, such as a racemic mixture.

In some embodiments, to the extent applicable, the genus of compounds in the present disclosure also excludes any of the compounds specifically prepared and disclosed prior to this disclosure.

### Method of Synthesis

The compounds of the present disclosure can be readily synthesized by those skilled in the art in view of the present disclosure. Exemplified synthesis is also shown in the Examples section.

The following synthetic processes of Formula I is illustrative. In some embodiments, the present disclosure also provides synthetic methods and synthetic intermediates for preparing the compounds of Formula I, as represented by the schemes herein.

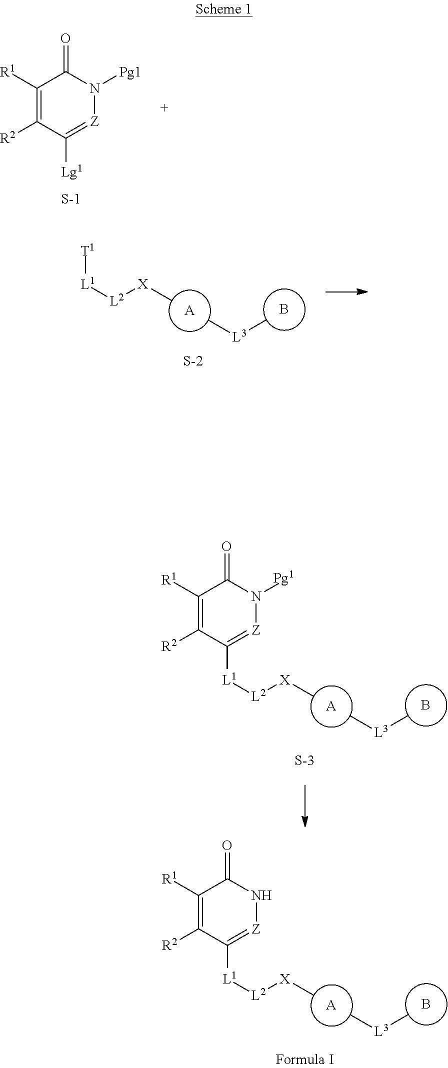

As shown in Scheme 1, compounds of Formula I can typically be synthesized through a coupling reaction of S-1 and S-2, followed by deprotection as needed. Typically, S-1 contains a leaving group, Lg¹, such as a halide, e.g., C₁, which can react with S-2, for example, when T¹ is hydrogen or a metal and L¹-T¹ has a nucleophilic functional group (e.g., OH, NH, SH, etc.) that can react with S-1 to form the desired link shown in S-3. In some embodiments, Pg¹ in S-1 is a protecting group, such as SEM (2-(trimethylsilyl) ethoxymethyl), and the synthesis of Formula I requires a deprotection of Pg¹ from S-3. In some embodiments, Pg¹ in S-1 can also be hydrogen, in which case, S-3 is a compound of Formula I. In some embodiments, the R¹ and/or R² in S-3 can be different from the counterpart in Formula I, in which case, further functional group transformations of S-3 may be carried out to obtain the target compound of Formula I. For example, in some embodiments, R¹ and/or R² may be a leaving group, which can be reacted under suitable situations to introduce a different R¹ and/or R² group. Exemplary reaction conditions for converting a compound of S-1 and S-2 into a compound of Formula I are shown in the Examples section. The variables R¹, R², L¹, L², L³, X, Z, ring A, and ring B in the formulae S-1, S-2, and S-3 of Scheme 1 include any of those defined hereinabove in connection with Formula I (e.g., any of the sub-formulae of Formula I) and protected derivatives thereof, when applicable.

In view of the present disclosure, it would also be apparent to those skilled in the art that compounds of Formula I can also be synthesized by different coupling strategies. For example, as shown in Scheme 2, S-4 can be coupled with S-5 under suitable conditions to form the L¹-L² link in S-3, which can then be optionally deprotected (when Pg¹ is a protecting group) and/or further functionalized to provide the desired compound of Formula I. For example, in some embodiments, L¹-T², optionally together with R¹ and R² in cases of a cyclic structure is formed among L¹, R¹ and R², can have a nucleophilic functional group (e.g., OH, NH, SH, etc.) that can react with S-5 to form the desired link shown in S-3, wherein T³ represents a precursor to L², which upon reaction with L¹-T² can provide the desired L¹-L² link in S-3. For example, in some embodiments, L¹-L² link in S-3 may contain O—C₁₋₄ alkylene or O-ethylene, in some embodiments, L¹-T² may contain an OH group, and T³ can represent a C₁₋₄ alkylene-Lg² or a vinyl group, wherein Lg² is a leaving group such as a halide, e.g., C₁, which upon reaction can provide the link containing O—C₁₋₄ alkylene or O-ethylene in S-3. Exemplary reaction conditions for converting a compound of S-4 and S-5 into a compound of Formula I are shown in the Examples section. The variables R¹, R², L¹, L², L³, X, Z, ring A, and ring B in the formulae S-4, S-5, and S-3 of Scheme 2 include any of those defined hereinabove in connection with Formula I (e.g., any of the sub-formulae of Formula I) and protected derivatives thereof, when applicable.

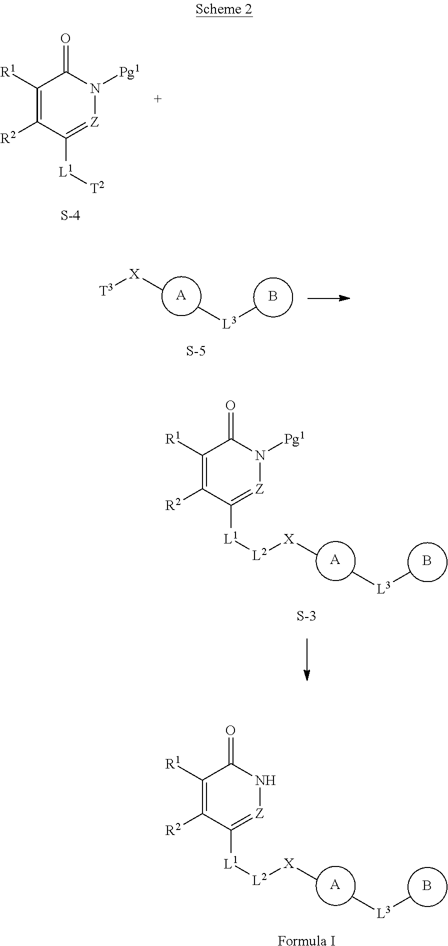

Similarly, as shown in Scheme 3, S-6 can be coupled with S-7 under suitable conditions to form the X link in S-3, which can then be optionally deprotected (when Pg¹ is a protecting group) and/or further functionalized to provide the desired compound of Formula I. For example, in some embodiments, L²-T⁴, optionally together with L¹ in cases of a cyclic structure is formed among L¹ and L², can have a X donor that can react with S-7 to form the desired link shown in S-3, wherein T⁵ represents hydrogen. For example, in some embodiments, the link X may be C(—O) group, and in some embodiments, L²-T⁴ may contain a COOH group, and Ring A-T⁵ can react with COOH under suitable conditions to provide the link X of C(═O) in S-3. Exemplary reaction conditions for converting a compound of S-6 and S-7 into a compound of Formula I are shown in the Examples section. The variables R¹, R², L′, L², L³, X, Z, ring A, and ring B in the formulae S-6, S-7, and S-3 of Scheme 3 include any of those defined hereinabove in connection with Formula I (e.g., any of the sub-formulae of Formula I) and protected derivatives thereof, when applicable.

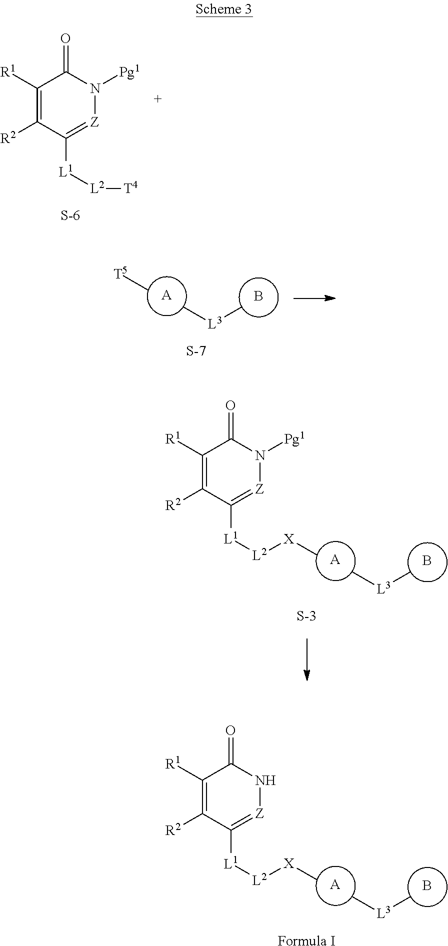

As would also be apparent to those skilled in the art, when Pg¹ in S-1, S-4, or S-6 is a protecting group, alternative protecting strategies masking the “amide” functional group can also be used. For example,

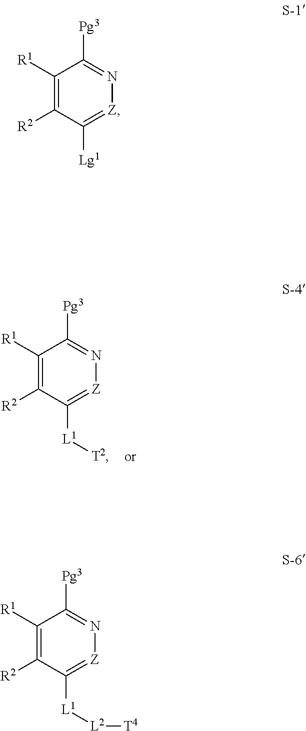

may be used in

Scheme 1, 2, or 3, in replacement of S-1, S-4, or S-6, respectively, to provide a counterpart S-3 and Formula I. Pg³ in S-1′, S-4′, or S-6′ can typically be a group that upon a hydrolysis reaction can yield the “C(O)—NH” functional group in Formula I. For example, in some embodiments, Pg³ can be C₁ or an alkoxy group such as methoxy or ethoxy.

Suitable coupling partners such as S-1, S-2, S-4, S-5, S-6, S-7, S-1′, S-4′, or S-6′ can be prepared by methods known in the art or methods in view of the present disclosure, see e.g., the Examples section.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4th ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7th Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more of the compounds of the present disclosure.

The pharmaceutical composition can optionally contain a pharmaceutically acceptable excipient. In some embodiments, the pharmaceutical composition comprises a compound of the present disclosure and a pharmaceutically acceptable excipient. Pharmaceutically acceptable excipients are known in the art. Non-limiting suitable excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., I-1, 1-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, or a pharmaceutically acceptable salt thereof, e.g., in a therapeutically effective amount. In any of the embodiments described herein, the pharmaceutical composition can comprise a therapeutically effective amount of a compound selected from compound Nos. 1-353, or a compound selected from Table A, or a pharmaceutically acceptable salt thereof.

The pharmaceutical composition can also be formulated for delivery via any of the known routes of delivery, which include but are not limited to oral, parenteral, inhalation, etc.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

In some embodiments, the pharmaceutical composition is formulated for inhalation. The inhalable formulations can be, for example, formulated as a nasal spray, dry powder, or an aerosol administrable through a metered-dose inhaler. Excipients for preparing formulations for inhalation are known in the art. Non-limiting suitable excipients include, for example, lactose, talc, silicic acid, aluminum hydroxide, calcium silicates and polyamide powder, and mixtures of these substances. Sprays can additionally contain propellants, such as chlorofluorohydrocarbons and volatile unsubstituted hydrocarbons, such as butane and propane.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as a cancer described herein, which can depend on the recipient of the treatment, the disease or disorder being treated and the severity thereof, the composition containing the compound, the time of administration, the route of administration, the duration of treatment, the compound potency (e.g., for inhibiting PARP7), its rate of clearance and whether or not another drug is co-administered.

For veterinary use, a compound of the present disclosure can be administered as a suitably acceptable formulation in accordance with normal veterinary practice. The veterinarian can readily determine the dosing regimen and route of administration that is most appropriate for a particular animal.

In some embodiments, all the necessary components for the treatment of PARP7-related disorder using a compound of the present disclosure either alone or in combination with another agent or intervention traditionally used for the treatment of such disease can be packaged into a kit. Specifically, in some embodiments, the present invention provides a kit for use in the therapeutic intervention of the disease comprising a packaged set of medicaments that include the compound disclosed herein as well as buffers and other components for preparing deliverable forms of said medicaments, and/or devices for delivering such medicaments, and/or any agents that are used in combination therapy with the compound of the present disclosure, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the internet.

### Method of Treatment

Compounds of the present disclosure are useful in inhibiting activity of PARPs in particular PARP7 in a cell or in a subject in need of inhibition of the enzyme. Compounds of the present disclosure are useful as therapeutic active substances for the treatment and/or prophylaxis of diseases or disorders that are associated with PARPs in particular PARP7.

As explained in WO2021/087018A1, WO2021/087025A1, and WO2019/212937, overexpression and/or activation of PARP7 was shown to have a role for cancer cells to evade host immune system through suppression of the Type I interferons and T cell mediated antitumor immunity. For example, it was stated therein that PARP7 knockout in a mouse melanoma cell line increased the proliferation and activation of co-cultured T cells. Thus, PARP7 inhibition can activate T cell mediated tumor killing.

In addition, recently, a PARP7 inhibitor is in Phase I clinical trial for patients with advanced or metastatic solid tumors. ClinicalTrials.gov Identifier: NCT04053673. As detailed in the clinical trial description, cancer cells use PARP7 to hide from the immune system by stopping the cell from sending a signal (Type 1 interferon) that tells the immune system that something is wrong and to kill the cell. According to the description in clinicaltrials.gov, the tested PARP7 inhibitor (RBN2397) has been shown in animal studies to inhibit tumor growth and also shuts down the “don't kill me” signal the tumor is sending to evade the immune system. These and other evidence further support the use of PARP7 inhibitors for use in treating various diseases associated with abnormal PARP7 expression and/or activities.

In some embodiments, the present disclosure provides a method of inhibiting PARP7, the method comprises contacting the PARP7 with an effective amount of one or more compounds of the present disclosure, such as a compound of Formula I (e.g., I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, or a pharmaceutically acceptable salt thereof.

In some embodiments, the present disclosure provides a method of inhibiting PARP7 in a cell, e.g., a cancer cell, the method comprising contacting the cell with an effective amount of one or more compounds of the present disclosure, such as a compound of Formula I (e.g., I-1, I-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, or a pharmaceutically acceptable salt thereof. In some embodiments, the cancer cell has an abnormal expression or activity of PARP7. In some embodiments, the cancer cell is in vitro. In some embodiments, the cancer cell is in vivo. In some embodiments, the cancer cell is a cell of the blood, breast, central nervous system, endometrium, kidney, large intestine, lung, oesophagus, ovary, pancreas, prostate, stomach, head and neck (upper aerodigestive), urinary tract, colon, and/or others.

In some embodiments, the present disclosure provides a method of treating cancer, the method comprising administering to a subject in need thereof a therapeutically effective amount of one or more compounds of the present disclosure, such as a compound of Formula I (e.g., I-1, 1-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, or a pharmaceutically acceptable salt thereof. In some embodiments, the cancer is breast cancer, cancer of the central nervous system, endometrium cancer, kidney cancer, large intestine cancer, lung cancer, esophagus cancer, ovarian cancer, pancreatic cancer, prostate cancer, stomach cancer, head and neck cancer (upper aerodigestive cancer), urinary tract cancer, or colon cancer. In some embodiments, the cancer is a hematopoietic malignancy such as leukemia and lymphoma. Examples of lymphomas include Hodgkin's or non-Hodgkin's lymphoma, multiple myeloma, B-cell lymphoma (e.g., diffuse large B-cell lymphoma (DLBCL)), chronic lymphocytic lymphoma (CLL), T-cell lymphoma, hairy cell lymphoma, and Burkett's lymphoma. Examples of leukemias include acute lymphocytic leukemia (ALL), acute myelogenous leukemia (AML), chronic lymphocytic leukemia (CLL), and chronic myelogenous leukemia (CML). In some embodiments, the cancer can be liver cancer (e.g., hepatocellular carcinoma), bladder cancer, bone cancer, glioma, breast cancer, cervical cancer, colon cancer, endometrial cancer, epithelial cancer, esophageal cancer, Ewing's sarcoma, pancreatic cancer, gallbladder cancer, gastric cancer, gastrointestinal tumors, head and neck cancer (upper aerodigestive cancer), intestinal cancers, Kaposi's sarcoma, kidney cancer, laryngeal cancer, lung cancer, prostate cancer, rectal cancer, skin cancer, stomach cancer, testicular cancer, thyroid cancer, and/or uterine cancer. In some embodiments, the cancer can be multiple myeloma, DLBCL, hepatocellular carcinoma, bladder cancer, esophageal cancer, head and neck cancer (upper aerodigestive cancer), kidney cancer, prostate cancer, rectal cancer, stomach cancer, thyroid cancer, uterine cancer, and/or breast cancer. In some embodiments, the cancer is associated with abnormal expression or activity of PARP7.

In some preferred embodiments, the compound of the present disclosure for the methods herein has a PARP7 IC50 or antiproliferation IC50 of less than 100 nM, as measured according to the Biological Assay Example A or B herein. In some preferred embodiments, the compound of the present disclosure for the methods herein is selected from the compounds according to Examples 1-353 that have a PARP7 IC50 or antiproliferation IC50 level designated as “A” or “B”, preferably “A”, in Table 2 and/or 3 herein.

PARP7-related disorders that can be treated with the methods herein also include those in disease areas such as cardiology, virology, neurodegeneration, inflammation, and pain, where the diseases are characterized by overexpression or increased activity of PARP7.

Compounds of the present disclosure can be used as a monotherapy or in a combination therapy. In some embodiments, the combination therapy includes treating the subject with a targeted therapeutic agent, chemotherapeutic or other anti-cancer agent, therapeutic antibody, radiation, cell therapy, anti-tumor and anti-viral vaccine, cytokine therapy, kinase inhibitor, epigenetic or signal transduction inhibitor, immune enhancer, immunosuppressant, and/or immunotherapy. In some embodiments, compounds of the present disclosure can also be co-administered with an additional pharmaceutically active compound, either concurrently or sequentially in any order, to a subject in need thereof. Any of the known therapeutic agents can be used in combination with the compounds of the present disclosure. In some embodiments, compounds of the present disclosure can also be used in combination with a radiation therapy, hormone therapy, cell therapy, surgery and/or immunotherapy, which therapies are well known to those skilled in the art.

Many chemotherapeutics are presently known in the art and can be used in combination with the compounds of the present disclosure. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Non-limiting examples are chemotherapeutic agents, cytotoxic agents, and non-peptide small molecules such as Gleevec® (Imatinib Mesylate), Kyprolis® (carfilzomib), Velcade® (bortezomib), Casodex (bicalutamide), Iressa® (gefitinib), venetoclax, and Adriamycin as well as a host of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel and docetaxel; retinoic acid; esperamicins; gemcitabine; capecitabine; and pharmaceutically acceptable salts, acids or derivatives of any of the above.

In some embodiments, the compounds of the present disclosure can be used in combination with anti-hormonal agents that act to regulate or inhibit hormone action on tumors such as anti-estrogens including for example tamoxifen, (Nolvadex™), raloxifene, aromatase inhibiting 4 (5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; 6-thioguanine; mercaptopurine; methotrexate; pemetrexed; platinum analogs such as cisplatin, carboplatin and oxaliplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO).

In some embodiments, the compounds or pharmaceutical composition of the present disclosure can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris (2-chloroethyl) amine, Troxacitabine, Uramustine, Vadimezan, Vinflunine, ZD6126 or Zosuquidar.

The compounds of the present disclosure may also be used in combination with an inhibitor of VEGF or VEGFR or kinase inhibitors of VEGFR. Inhibitors of VEGFR kinases and other anti-angiogenesis inhibitors include but are not limited to sunitinib, sorafenib, axitinib, cediranib, pazopanib, regorafenib, brivanib, and vandetanib.

The compounds of the present disclosure may also be used in combination with an inhibitor of FGFR inhibitors.

The compounds or pharmaceutical compositions of the disclosure can also be used in combination with an amount of one or more substances selected from EGFR inhibitors, CDK inhibitors, MEK inhibitors, PI3K inhibitors, AKT inhibitors, TOR inhibitors, Mcl-1 inhibitors, BCL-2 inhibitors, SHP2 inhibitors, proteasome inhibitors, and immune therapies, including monoclonal antibodies, immunomodulatory imides (IMiDs), anti-PD-1, anti-PDL-1, anti-CTLA4, anti-LAG1, and anti-OX40 agents, GITR agonists, CAR-T cells, and BiTEs.

In some embodiments, the compounds of the present disclosure may also be used in combination with an immunotherapy, such as a PD-1 and PD-L¹ antagonist, such as an anti-PD-1 or anti-PDL-1 antibody, or anti-CTLA-4 or anti-4-1BB antibodies, etc. Exemplary anti-PD-1 or anti-PDL-1 antibodies and methods for their use are described by Goldberg et al., Blood 110 (1): 186-192 (2007), Thompson et al., Clin. Cancer Res. 13 (6): 1757-1761 (2007), and Korman et al., International Application No. PCT/JP2006/309606 (publication no. WO 2006/121168 A1), each of which are expressly incorporated by reference herein.

Examplary immunotherapies that can be used in combination with the compounds or compositions of the present disclosure include: pembrolizumab (Keytruda®), nivolumab (Opdivo®), Yervoy™ (ipilimumab) or Tremelimumab (to CTLA-4), galiximab (to B7.1), M7824 (a bifunctional anti-PD-L1/TGF-β Trap fusion protein), AMP224 (to B7DC), BMS-936559 (to B7-H1), MPDL3280A (to B7-H1), MEDI-570 (to ICOS), AMG 404, AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4).

Suitable immune therapies for combined use with the compounds or compositions of the present disclosure also include genetically engineered T-cells (e.g., CAR-T cells) and bispecific antibodies (e.g., BiTEs).

Non-limiting useful additional agents for combined use with the compounds or compositions of the present disclosure also include anti-EGFR antibody and small molecule EGFR inhibitors such as cetuximab (Erbitux), panitumumab (Vectibix), zalutumumab, nimotuzumab, matuzumab, gefitinib, erlotinib (Tarceva), lapatinib (TykerB), etc. Non-limiting useful additional agents also include CDK inhibitors such as CDK4/6 inhibitors, such as seliciclib, UCN-01, P1446A-05, palbociclib (PD-0332991), abemaciclib, dinaciclib, P27-00, AT-7519, RGB286638, and SCH727965, etc. Non-limiting useful additional agents also include MEK inhibitors such as trametinib (MekinistR), CI-1040, AZD6244, PD318088, PD98059, PD334581, RDEA119, ARRY-142886, ARRY-438162, and PD-325901.

Additional useful agents that may be combined with the compounds or compositions of the present disclosure include the additional pharmaceutical agents described in the Combination Therapy section in WO2021/087018A1, WO2021/087025A1, or WO2019/212937.

The administering herein is not limited to any particular route of administration. For example, in some embodiments, the administering can be orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the administering is orally.

Dosing regimen including doses can vary and can be adjusted, which can depend on the recipient of the treatment, the disease or disorder being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable atoms or groups for the variables herein are independently selected. The definitions of the variables can be combined. Using Formula I as an example, any of the definitions of one of R¹, R², L¹, L², L³, X, Z, ring A, and ring B in Formula I can be combined with any of the definitions of the others of R¹, R², L¹, L², L³, X, Z, ring A, and ring B in Formula I. Such combination is contemplated and within the scope of the present disclosure.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds of the present disclosure can comprise one or more asymmetric centers and/or axial chirality, and thus can exist in various isomeric forms, e.g., enantiomers and/or diastereomers. For example, the compounds described herein can be in the form of an individual enantiomer, diastereomer, atropisomer, or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC), SFC, and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, I N 1972). The disclosure additionally encompasses compounds described herein as individual isomers substantially free of other isomers, and alternatively, as mixtures of various isomers including racemic mixtures. In embodiments herein, unless otherwise obviously contrary from context, when a stereochemistry is specifically drawn, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s), for example, the compound can have an enantiomeric excess (“ee”) of greater than 80%, such as greater than 90% ee, greater than 95% ee, greater than 98% ee, greater than 99% ee, or the other enantiomer is non-detectable. The presence and/or amounts of stereoisomers can be determined by those skilled in the art in view of the present disclosure, including through the use of chiral HPLC or SFC. It should also be understood that for any compound of the present disclosure herein with its stereochemistry specifically drawn, the corresponding racemic mixture or stereoisomeric mixture in any ratio is also contemplated by the present disclosure, such racemic mixture or stereoisomeric mixture is also compounds of the present disclosure.

When a range of values is listed, it is intended to encompass each value and sub-range within the range. For example, “C₁₋₆” is intended to encompass, C₁, C₂, C₃, C₄, Cs, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆. As used herein, the term “compound(s) of the present disclosure” or

“compound(s) of the present invention” refers to any of the compounds described herein according to Formula I (e.g., I-1, 1-2, I-A, I-B, I-A-a, I-A-1, I-A-2, I-A-3, I-A-1-a, I-B-1, I-B-2, I-B-3, I-B-1-E1, I-B-1-E2, I-B-1-E1-a, I-B-1-E2-a, I-B-2-E1, I-B-2-E2, I-B-3-E1, I-B-3-E2, I-C-1, I-C-1-a, I-C-1-al, I-C-1-a2, I-C-2, I-C-3, I-C-1-E1, I-C-1-E2, I-C-1-E3, I-C-1-E4, I-C-2-E1, I-C-2-E2, I-C-2-E3, I-C-2-E4, I-C-3-E1, I-C-3-E2, I-C-3-E3, I-C-3-E4, I-D-1, I-D-2, I-D-3, I-D-1-a, I-D-2-a, I-D-3-a, I-D-1-b, I-D-2-b, I-D-3-b, or I-D-1-c), any of Compound Nos. 1-353, any of the compounds disclosed in Table A herein, isotopically labeled compound(s) thereof (such as a deuterated analog wherein one or more of the hydrogen atoms is substituted with a deuterium atom with an abundance above its natural abundance), possible stereoisomers thereof (including diastereoisomers, enantiomers, and racemic mixtures), geometric isomers thereof, atropisomers thereof, tautomers thereof, conformational isomers thereof, and/or pharmaceutically acceptable salts thereof (e.g., acid addition salt such as HCl salt or base addition salt such as Na salt). For the avoidance of doubt, Compound Nos. 1-353 or Compounds 1-353 refers to the compounds described herein labeled as integers 1, 2, 3, . . . , 353, see for example the title compounds of Examples and Table 1. For ease of description, synthetic starting materials or intermediates may be labeled with an integer (compound number) followed by a “−” and additional numeric values, such as 1-1, 1-2, etc., see examples for details. The labeling of such synthetic starting materials or intermediates should not be confused with the compounds labeled with an integer only without the “−” and additional numeric value. Some of compounds 1-353 refer to separated enantiomers, for example, through SFC methods described in the Examples section. The absolute stereochemistry for these separated enantiomers is not determined. If the assumed stereochemistry in these separated enatiomers as described in the Examples section is incorrect, those skilled in the art would understand that the correct stereochemistry should then be the opposite enantiomer of the assumed. In any event, these separated enantiomers can also be characterized by their retention times in the chiral SFC methods described herein and their biological activities such as in inhibiting PARP7 as described herein. It should also be apparent that the corresponding racemic mixtures of these separated enantiomers are also compounds of the present disclosure. Hydrates and solvates of the compounds of the present disclosure are considered compositions of the present disclosure, wherein the compound(s) is in association with water or solvent, respectively. In some embodiments, compounds of the present disclosure refer to any of the compounds according to claims 1-86 herein or a pharmaceutically acceptable salt thereof. In some embodiments, compounds of the present disclosure refer to any of the compounds according to exemplified Embodiments 1-28 herein or a pharmaceutically acceptable salt thereof.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to 2H, 3H, 13C, 14C, 15N, 180, 32P, 35S, 18F, 36Cl, and 1251. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₁₂ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. In one embodiment, the alkyl group is a C₁₋₄ alkyl group selected from methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, such as one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, such as one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl as defined herein. As used herein, the term “cycloalkoxy” as used by itself or as part of another group refers to a radical of the formula OR^{a1}, wherein R^{a1} is a cycloalkyl as defined herein. As used herein, the term “heterocycloalkoxy” as used by itself or as part of another group refers to a radical of the formula OR^{a1}, wherein R^{a1} is a heterocyclyl group as defined herein.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of which has been replaced by a heteroatom selected from S, O. P and N, and wherein the nitrogen, phosphine, and sulfur atoms can optionally be oxidized and the nitrogen heteroatom can optionally be quaternized. The heteroatom(s) S, O. P and N may be placed at any interior position of the heteroalkyl group or at the position at which the alkyl group is attached to the remainder of the molecule. For example, C₁₋₄ heteroalkyl include but are not limited to, C₄ heteroalkyl such as —CH₂—CH₂—N(CH₃)—CH₃, C₃ heteroalkyl such as —CH₂—CH₂—O—CH₃,—CH₂—CH₂—NH—CH₃, —CH₂—S—CH₂—CH₃, —CH₂—CH₂—S(O)—CH₃, and —CH₂—CH₂—S(O)₂—CH₃, C₂ heteroalkyl such as —O—CH₂—CH₃ and C₁ heteroalkyl such as O—CH₃, etc. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having from 3 to 10 ring carbon atoms (“C₃₋₁₀ carbocyclyl”) and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring.

“Heterocyclyl” or “heterocyclic” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). Heterocyclyl or heterocyclic ring that has a ring size different from the 3-10 membered heterocyclyl is specified with a different ring size designation when applicable. Those skilled in the art would understand that such different ring-sized heterocyclyl is also a non-aromatic ring system having ring carbon atoms and 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic)4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). Heteroaryl that has a ring size different from the 5-10 membered heteroaryl is specified with a different ring size designation when applicable. Those skilled in the art would understand that such different ring-sized heteroaryl is also a 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur. In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl).

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzothiazolyl, benzisothiazolyl, benzothiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

As commonly understood by those skilled in the art, alkylene, alkenylene, alkynylene, carbocyclylene, heterocyclylene, arylene, and heteroarylene refer to the corresponding divalent radicals of alkyl, alkenyl, alkynyl, carbocyclyl, heterocyclyl, aryl, and heteroaryl groups, respectively.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkenyl, alkynyl, carbocyclic, cycloalkyl, alkoxy, cycloalkoxy, or heterocyclic group herein can be unsubstituted or substituted with 1, 2, 3, or 4 substituents independently selected from F, Cl, —OH, protected hydroxyl, oxo (as applicable), NH₂, protected amino, NH (C₁₋₄ alkyl) or a protected derivative thereof, N(C₁₋₄ alkyl ((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms, each independently selected from O, S, and N, 3-7 membered heterocyclyl containing 1 or 2 ring heteroatoms, each independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl or heteroaryl group herein can be unsubstituted or substituted with 1, 2, 3, or 4 substituents independently selected from F, Cl, —OH, —CN, NH₂, protected amino, NH (C₁₋₄ alkyl) or a protected derivative thereof, N(C₁₋₄ alkyl ((C₁₋₄ alkyl), —S(═O) (C₁₋₄ alkyl), —SO₂ (C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms, each independently selected from O, S, and N, 3-7 membered heterocyclyl containing 1 or 2 ring heteroatoms, each independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃X, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O) R^{aa}, —OCO₂R^{aa}, —C(═O) N(R^{bb})₂, —OC(═O) N(R^{bb})₂, —NR^{bb}C(═O) R^{aa}, —NR^{bb}CO₂R^{aa}, —NR^{bb}C(═O) N(R^{bb})₂, —C(═NR^{bb}) R^{aa}, —C(═NR^{bb}) OR^{aa}, —OC(═NR^{bb}) R^{aa}, —OC(═NR^{bb}) OR^{aa}, —C(═NR^{bb}) N(R^{bb})₂, —OC(═NR^{bb}) N(R^{bb})₂, —NR^{bb}C(═NR^{bb}) N(R^{bb})₂, —C(═O) NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O) R^{aa}, —OS(═O)R^{aa}, —Si(R^{aa})₃, —OSi(R^{aa})₃—C(═S) N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(RC)₂, —P(OR^{cc})₂, —P(RC)₃X, —P(OR^{cc})₃X, —P(R^{cc})₄, —P(OR^{aa})₄, —OP(R^{cc})₂, —OP(R^{cc})₃X, —OP(OR^{cc})₂, —OP(OR^{cc})₃X, —OP(R^{Cc})₄, —OP(OR^{cc})₄, —B(R^{aa})₂, —B(OR^{cc})₂, —BR^{aa}(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; wherein X is a counterion;

or two geminal hydrogens on a carbon atom are replaced with the group ═O, ═S, ═NN(R^{bb})₂, ═NNR^{bb}C(═O) R^{aa}, ═NNR^{bb}C(═O) OR^{aa}, ═NNR^{bb}s (═O)₂R^{aa}, ═NR^{bb}, or ═NOR^{cc}; each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O) R^{aa}, —C(═O) N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S) N(R^{cc})₂, —C(═O)SR^{aa}, —C(═S)SR^{aa}, —P(═O)(R^{aa})₂, —P(═O)(OR^{cc})₂, —P(═O)(N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; wherein X is a counterion;

each instance of Rec is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃X, —N(OR^{ee}) R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{f})₂, —NR^{ff}C(═O)R^{ee}, —NR^{ff}CO₂R^{ee}, —NR^{ff}C(═O) N(R^{ff})₂, —C(═NR^{ff}) OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R)₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X is a counterion;

each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R⁸⁸ groups;
each instance of R^{ff} is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R⁸⁸ groups; and
each instance of R⁸⁸ is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃X, —NH (C₁₋₆ alkyl)₂X, —NH₂ (C₁₋₆ alkyl) X, —NH₃X, —N(OC₁₋₆ alkyl) (C₁₋₆ alkyl), —N(OH) (C₁₋₆ alkyl), —NH(OH), —SH, —SC₁₋₆ alkyl, —SS(C₁₋₆ alkyl), —C(═O) (C₁₋₆ alkyl), —CO₂H, —CO₂ (C₁₋₆ alkyl), —OC(═O) (C₁₋₆ alkyl), —OCO₂ (C₁₋₆ alkyl), —C(═O) NH₂, —C(═O) N(C₁₋₆ alkyl)₂, —OC(═O) NH (C₁₋₆ alkyl), —NHC(═O) (C₁₋₆ alkyl), —N(C₁₋₆ alkyl) C(═O) (C₁₋₆ alkyl), —NHCO₂ (C₁₋₆ alkyl), —NHC(═O) N(C₁₋₆ alkyl)₂, —NHC(═O) NH (C₁₋₆ alkyl), —NHC(═O) NH₂, —C(═NH) O(C₁₋₆ alkyl), —OC(═NH) (C₁₋₆ alkyl), —OC(═NH) OC₁₋₆ alkyl, —C(═NH) N(C₁₋₆ alkyl)₂, —C(═NH) NH (C₁₋₆ alkyl), —C(═NH) NH₂, —OC(═NH) N(C₁₋₆ alkyl)₂, OC(NH) NH (C_{1 6} alkyl), —OC(NH) NH₂, —NHC(NH) N(C₁₋₆ alkyl)₂, —NHC(═NH) NH₂, —NHSO₂ (C₁₋₆ alkyl), —SO₂N(C₁₋₆ alkyl)₂, —SO₂NH (C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S) N(C₁₋₆ alkyl)₂, C(═S) NH (C₁₋₆ alkyl), C(═S) NH₂, —C(═O) S(C₁₋₆ alkyl), —C(═S) SC₁₋₆ alkyl, —SC(═S) SC₁₋₆ alkyl, —P(═O) (OC₁₋₆ alkyl)₂, —P(═O) (C₁₋₆ alkyl)₂, —OP(═O) (C₁₋₆ alkyl)₂, —OP(═O) (OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R⁸⁸ substituents can be joined to form-O or ═S; wherein X is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HSO₄, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B [3,5-(CF₃)₂C₆H₃]₄], BPh₄, Al(OC(CF₃)₃)₄⁻, and a carborane anion (e.g., CB₁₁H₁₂ or (HCB₁₁Me₅Br₆)⁻). Exemplary counterions which may be multivalent include CO₃²⁻, HPO₄²⁻, PO₄³⁻. B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

“Acyl” refers to a moiety selected from the group consisting of —C(═O) R^{aa}, —CHO, —CO₂R^{aa}, —C(═O) N(R^{bb})₂, —C(═NR^{bb}) R^{aa}, —C(═NR^{bb}) OR^{aa}, —C(═NR^{bb}) N(R^{bb})₂, —C(═O) NR^{bb}SO₂R^{aa}, −C(═S) N(R^{bb})₂, —C(═O) SR^{aa}, or —C(═S) SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O) R^{aa}, —C(═O) N(R^{cc})₂, —CO₂R^{aa}, —SO₂R^{aa}, —C(═NR^{bb}) R^{aa}, —C(═NR^{cc}) OR^{aa}, —C(═NR^{cc}) N(R^{cc})₂, —SO₂N(R^{cc})₂, —SO₂R^{cc}, —SO₂OR^{cc}, —SOR^{aa}, —C(═S) N(R^{cc})₂, —C(═O) SR^{cc}, —C(═S) SR^{cc}, —P(═O) (OR_{cc})₂, —P(═O) (R^{aa})₂, —P(═O) (N(R^{cc})₂)₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two Rec groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, Rec, and R^{dd} are as defined above.

In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, —R^{aa}, —C(═O) SR^{aa}, —C(═O) R^{aa}, —CO₂R^{aa}, —C(═O) N(R^{bb})₂, —C(═NR^{bb}) R^{aa}, —C(═NR^{bb}) OR^{aa}, —C(═NR^{bb}) N(R^{bb})₂, —S(═O) R^{aa}, —SO₂R^{aa}, —Si(R^{aa})₃, —P(R^{cc})₂, —P(R^{cc})₃X, —P(OR^{cc})₂, —P(OR^{cc})₃X, —P(═O) (R^{aa})₂, —P(═O) (OR^{cc})₂, and —P(═O) (N(R^{bb})₂)₂, wherein X, R^{aa}, R^{bb}, and R^{cc} are as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3rd edition, John Wiley & Sons, 1999, incorporated herein by reference. Exemplary oxygen protecting groups include, but are not limited to, alkyl ethers or substituted alkyl ethers such as methyl, allyl, benzyl, substituted benzyls such as 4-methoxybenzyl, methoxymethyl (MOM), benzyloxymethyl (BOM), 2-methoxyethoxymethyl (MEM), etc., silyl ethers such as trymethylsilyl (TMS), triethylsilyl (TES), triisopropylsilyl (TIPS), t-butyldimethylsilyl (TBDMS), etc., acetals or ketals, such as tetrahydropyranyl (THP), esters such as formate, acetate, chloroacetate, dichloroacetate, trichloroacetate, trifluoroacetate, methoxyacetate, etc., carbonates, sulfonates such as methanesulfonate (mesylate), benzylsulfonate, and tosylate (Ts), etc.

The term “leaving group” is given its ordinary meaning in the art of synthetic organic chemistry, for example, it can refer to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O)-dimethylhydroxylamino, pixyl, and haloformates.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses.

Exemplary embodiments of steps for performing the synthesis of products described herein are described in greater detail infra. Some of the Examples discussed herein can be prepared by separating from the corresponding racemic mixtures. As would be understood by a person of ordinary skill in the art, the compounds described in the Examples section immmmediately prior to the chiral separation step, e.g., by supercritical fluid chromatography (SFC), exist in racemic and/or stereoisomeric mixture forms. It should be understood that the enantiomeric excesses (“ee”) reported for these examples are only representative from the exemplified procedures herein and not limiting; those skilled in the art would understand that such enantiomers with a different ee, such as a higher ee, can be obtained in view of the present disclosure.

The abbreviations used in the Examples section should be understood as having their ordinary meanings in the art unless specifically indicated otherwise or obviously contrary from context.

| Abbreviations | Chemical Name |
|---|---|
| Pd(OAc)₂ | Palladium (II) acetate |
| AcOH/HOAc | Acetic acid |
| AcONa/NaOAc | Sodium acetate |
| DMF | N,N-dimethylformamide |
| NMP | N-methylpyrrolidin-2-one |
| THF | Tetrahydrofuran |
| TFA | Trifluoroacetic acid |
| FA | Formic acid |
| SFC | Supercritical Fluid Chromatography |
| DMAc | N,N-dimethylacetamide |
| TEA | Triethylamine |
| DEA | Diethylamine |
| SEMCl | 2-(Trimethylsilyl)ethoxymethyl Chloride |
| SEM | 2-(Trimethylsilyl)ethoxymethyl |
| TIPSCl | Triisopropylsilyl Chloride |
| TIPS | Triisopropylsilyl |
| KOAc | Potassium acetate |
| TBSOTf | tert-Butyldimethylsilyl trifluoromethanesulfonate |
| TBAF | Tetra-n-butylammonium fluoride |
| mCPBA | 3-Chloroperbenzoic acid |
| TMSI | Trimethyliodosilane |
| Pd2(dba)3 | Tris(dibenzylideneacetone)dipalladium(0) |
| Me4t—BuXPhos | 2-Di-t-butylphosphino-3,4,5,6-tetramethyl-2′,4′,6′-tri- |
| | i-propyl)-1,1′-biphenyl |
| HATU | O-(7-Azabenzotriazol-1-yl)-N,N,N′,N′- |
| | tetramethyluronium hexafluorophosphate |
| BINAP | racemic-2,2′-Bis(diphenylphosphino)-1,1′-binaphthyl |
| DBU | 1,8-Diazabicyclo[5.4.0]undec-7-ene |
| DIEA | N,N-Diisopropylethylamine |
| EDCI | N-Ethyl-N′-(3-dimethylaminopropyl)carbodiimide |
| | hydrochloride |
| DMAP | 4-Dimethylaminopyridine |
| Xantphos | 9,9-Dimethyl-4,5-bis(diphenylphosphino)xanthene |
| NBS | N-Bromosuccinimide |
| AIBN | 2,2′-Azobis(2-methylpropionitrile) |
| DCM | dichloromethane |
| LDA | Lithium diisopropylamide |
| DAST | Diethylaminosulphur trifluoride |
| TBDPSCl | tert-Butylchlorodiphenylsilane |
| Ruphos Pd G3 | (2-Dicyclohexylphosphino-2′,6′-diisopropoxy-1,1′- |
| | biphenyl)[2-(2′-amino-1,1′-biphenyl)]palladium(II) |
| | methanesulfonate |
| T₃P | Propanephosphonic acid cyclic anhydride |

### Example 1 Synthesis of Compound 1, 2, and 3

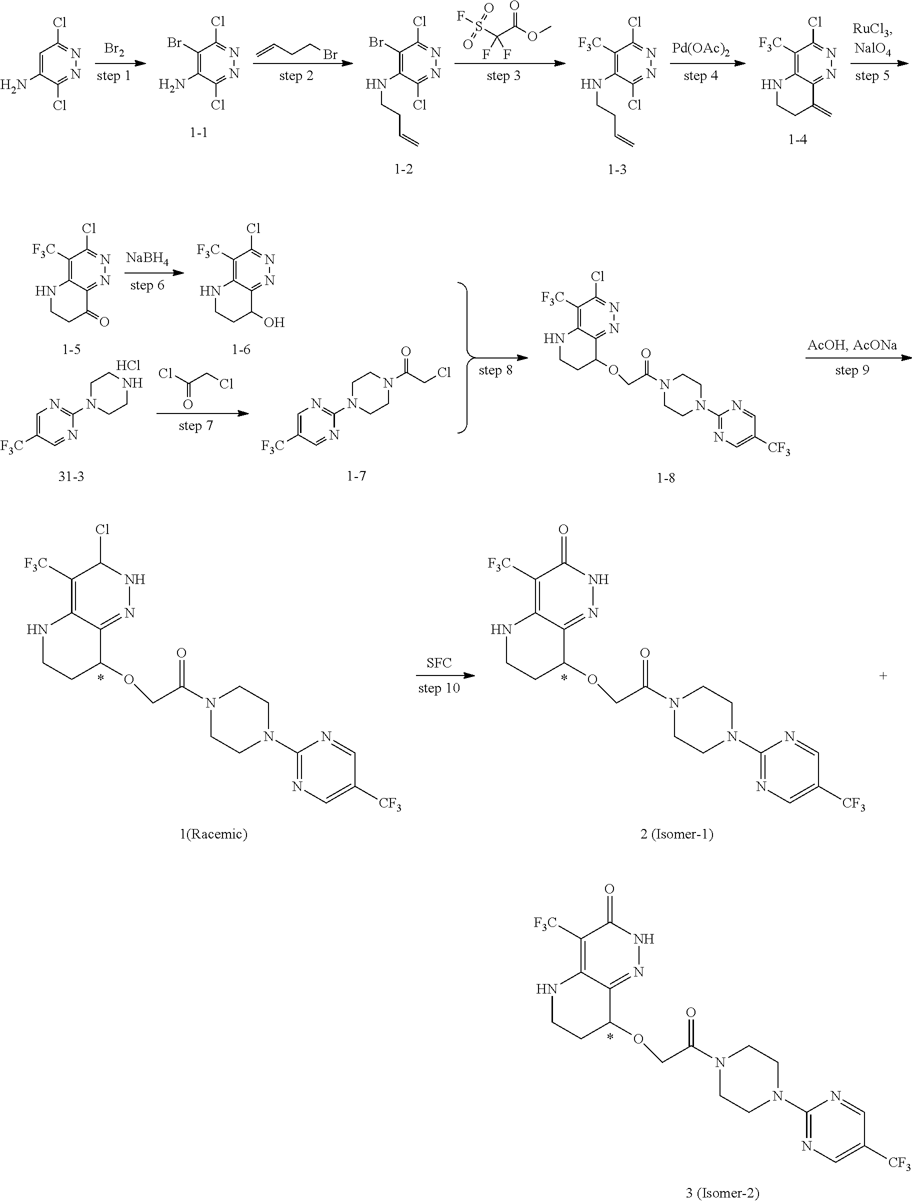

Step 1: To a solution of 3,6-dichloropyridazin-4-amine (21.8 g, 133 mmol) and NaOAc (21 g, 266 mmol) in CH₃CN(200 mL) at 80° C. was added a solution of Br₂ (43 g, 266 mmol) in CH₃CN(50 mL) dropwise, stirred for 1 h. The mixture was cooled, diluted with tetrahydrofuran and ethyl acetate. The organic layer was separated and washed with aqueous NaHCO₃, dried over anhydrous sodium sulfate, filtered and concentrated. The residue was stirred in methyl tert-butyl ether for 30 min, filtered, and the solid was dried to afford 1-1.

Step 2: To a solution of 1-1 (20 g, 82.3 mmol) in DMF (200 mL) were added Cs₂CO₃ (53.6 g, 164.6 mmol) and 4-bromobut-1-ene (12.2 g, 90.5 mmol). The reaction was stirred at 65° C. for 16 h under nitrogen. The mixture was cooled, diluted with ethyl acetate and washed with water. The organic layer was dried over anhydrous sodium sulfate, filtered and concentrated. The residue was taken up in dichloromethane and stirred for 30 min. The mixture was filtered and the solid was collected and dried to afford 1-2. The filtrate was concentrated and the residue was purified by silica gel column chromatography (petroleum ether to petroleum ether/ethyl acetate=2/1) to afford another batch of 1-2.

Step 3: To a solution of 1-2 (1 g, 3.4 mmol) in NMP (25 mL) were added Cul (1.5 g, 8 mmol) and methyl 2,2-difluoro-2-(fluorosulfonyl) acetate (1.5 g, 8 mmol). The mixture was stirred in a preheated oil bath at 105° C. for 1 h under N₂. After being cooled to room temperature, the mixture was filtered through a Celite pad. The filtrate was diluted with ethyl acetate and washed with water. The organic layer was dried over anhydrous sodium sulfate, filtered and concentrated. The residue was purified by silica gel column chromatography (petroleum ether to petroleum ether/ethyl acetate=3/1) to afford 1-3.

Step 4: To a solution of 1-3 (460 mg, 1.6 mmol) in DMAc (15 mL) were added Pd(OAc)₂ (36 mg, 0.16 mmol), P (o-Tolyl)₃ (122 mg, 0.4 mmol), and KOAc (980 mg, 10 mmol) under N₂. The mixture was stirred at 80° C. for 20 min under microwave condition. The mixture was cooled, diluted with ethyl acetate and washed with water and saturated aqueous NaHCO₃ (30 mL). The organic layer was separated and dried over anhydrous sodium sulfate, filtered and concentrated. The residue was purified by silica gel column chromatography (petroleum ether to petroleum ether/ethyl acetate=1/1) to afford 1-4.

Step 5: To a solution 1-4 (80 mg, 0.32 mmol) in CH₃CN(4 mL) and CCl₄ (4 mL) was added a solution of NaIO₄ (204 mg, 0.96 mmol) in H₂O (4.5 mL), followed by a solution of RuCl₃ (6.6 mg, 0.032 mmol) in H₂O (1.5 mL). The mixture was stirred at room temperature for 2.5 h under N₂, then diluted with dichloromethane and water. The organic layer was dried over anhydrous sodium sulfate, filtered and concentrated. The residue was purified by silica gel column chromatography (petroleum ether to petroleum ether/ethyl acetate=1/3) to afford 1-5.

Step 6: To a solution of 1-5 (46 mg, 0.09 mol) in THF (6 mL) at 0° C. was added NaBH₄ (38 mg, 1 mmol) under N₂. The reaction was stirred at room temperature for 6 h, quenched with a solution of AcOH (240 mg, 4 mol) in THF (2 mL) and saturated aqueous NH₄Cl (10 mL). The resulting mixture was diluted with water and extracted with ethyl acetate. The organic layer was dried over anhydrous sodium sulfate, filtered and concentrated. The residue was purified by prep-HPLC (acetonitrile with 0.05% of TFA in water: 15% to 45%) to afford 1-6.

Step 7: To a solution of 31-3 (20.0 g, 66 mmol) in dichloromethane (200 mL) was added TEA (26.5 g, 262.4 mmol) and chloroacetyl chloride (8.2 g, 72.1 mmol) at 0° C. The reaction mixture was stirred at room temperature for 5 h, diluted with water and extracted with ethyl acetate. The combined organic layers were washed with brine, dried over Na₂SO₄, filtered and concentrated. The residue was purified by silica gel column chromatography (petroleum ether/ethyl acetate=3/1) to afford 1-7.

Step 8: To a solution of 1-6 (100 mg, 0.3 eq TFA salt, 0.36 mmol) in DMF (20 mL) was added NaH (80 mg, 60% in oil, 2 mmol) at 0° C. under N₂. The reaction mixture was stirred at 0° C. for 10 min and then stirred at room temperature for 0.5 h. The mixture was cooled to 0° C., a solution of 1-7 (145 mg, 0.48 mmol) in DMF (6 mL) was added. The reaction was stirred at room temperature for 16 h under N₂ before it was cooled to 0° C., diluted with ethyl acetate and quenched with saturated aqueous NH₄Cl (30 mL). The organic layer was washed with H₂O and brine, dried over anhydrous sodium sulfate, filtered and concentrated. The residue was purified by prep-HPLC (acetonitrile with 0.05% of TFA in water: 15% to 45%) to afford 1-8.

Step 9: To a solution of 1-8 (15 mg, 0.028 mmol) in AcOH (4 mL) was added NaOAc (23 mg, 0.28 mmol). The mixture was stirred at 100° C. for 3 h. The mixture was cooled, diluted with water and extracted with ethyl acetate. The combined organic layers were dried over anhydrous sodium sulfate, filtered and concentrated. The residue was purified by prep-HPLC (acetonitrile with 0.05% of TFA in water: 15% to 45%) to afford 1. LCMS (ESI, m z): [M+H]=508.2; ¹H NMR (400 MHZ, DMSO-d₆) δ 12.34 (s, 1H), 8.72 (d, J=0.8 Hz, 2H), 7.55 (d, J=2.0 Hz, 1H), 4.37 (m, J=2.8 Hz, 1H), 4.27 (s, 2H), 3.85-3.77 (m, 5H), 3.55-3.43 (m, 5H), 2.18-2.15 (m, 1H), 1.89-1.81 (m, 1H). ¹⁹F NMR (376 MHZ, DMSO-d₆)8-55.68 (3F), -59.32 (3F).

Step 10: Racemic compound 1 was separated by SFC (column: DAICEL CHIRALCELROZ, MeOH (+0.1% 7.0 M ammonia in MeOH)/CO₂=60/40) to afford 2 (peak 1) and 3 (peak2) respectively. SFC analysis of 2: >99% ee; retention time: 4.39 min; column: DAICEL CHIRALCELROZ, MeOH (0.1% of DEA) in CO₂, 5% to 40%; pressure: 100 bar; flow rate: 1.5 mL/min. SFC analysis of 3: >99% ee; retention time: 4.79 min; column: DAICEL CHIRALCEL®OZ, MeOH (0.1% of DEA) in CO₂, 5% to 40%; pressure: 100 bar; flow rate: 1.5 mL/min.

### Example 2 Synthesis of Compounds 4 and 5

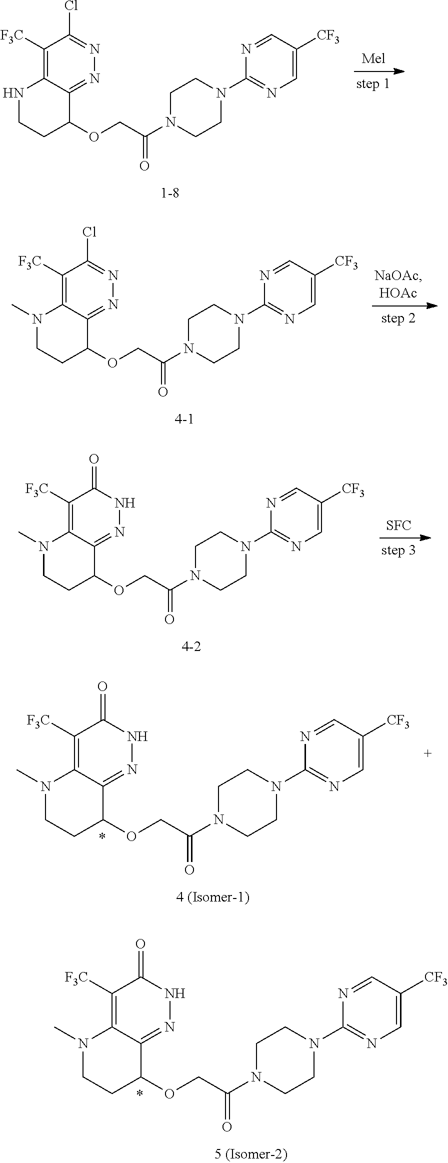

Step 1: To a stirred solution of 1-8 (55 mg, 0.11 mmol) in DMF (1 mL) was added NaH (60%, 6 mg, 0.15 mmol) at room temperature under N₂. The mixture was stirred at room temperature for 1 h before Mel (22 mg, 0.16 mmol) was added dropwise, and then stirred overnight. The reaction was quenched with H₂O and extracted with ethyl acetate. The combined organic layers were dried over anhydrous sodium sulfate, filtered and concentrated. The residue was purified by prep-TLC (ethyl acetate) to afford 4-1.

Step 2: Under N₂, 4-1 (45 mg), CH₃COOH (90 mg, 1.50 mmol), and CH₃COONa (90 mg, 1.10 mmol) were mixed in DMAc (2 mL). The reaction mixture was stirred at 110° C. for 60 h, cooled, quenched with H₂O and extracted with ethyl acetate. The combined organic layers were dried over anhydrous sodium sulfate, filtered and concentrated. The residue was purified by prep-TLC (dichloromethane/methanol=20/1) to afford 4-2.

Step 3:4-2 was purified by SFC (column: Chiral-IG, MeOH/CO₂=30/70) to afford 4 (peak-1, 4.2 mg) and 5 (peak-2, 5.3 mg) respectively. SFC analysis of 4: >99% ee; retention time: 10.86 min; column: Chiral-IG, MeOH in CO₂, 40%; pressure: 100 bar; flow rate: 2.0 mL/min. LCMS (ESI, m z): [M+H]⁺=522.0; ¹H NMR (400 MHZ, methanol-d₄, ppm): δ 8.60 (s, 2H), 4.86 (s, 2H), 3.99-3.89 (m, 4H), 3.66-3.54 (m, 5H), 3.41-3.40 (m, 1H), 3.16-3.14 (m, 3H), 2.38-2.34 (m, 1H), 2.04-2.01 (m, 1H). ¹⁹F NMR (376 MHz, methanol-d₄, ppm): 8-56.47 (3F), -62.65 (3F). SFC analysis of 5:99.3% ee; retention time: 12.58 min; column: Chiral-IG, MeOH in CO₂, 40%; pressure: 100 bar; flow rate: 2.0 mL/min. LCMS (ESI, m z): [M+H]⁺=522.0; ¹H NMR (400 MHZ, methanol-d₄, ppm): δ 8.60 (s, 2H), 4.86 (s, 2H), 4.00-3.90 (m, 4H), 3.66-3.56 (m, 5H), 3.41-3.40 (m, 1H), 3.16-3.15 (m, 3H), 2.38-2.35 (m, 1H), 2.04-2.01 (m, 1H). ¹⁹F NMR (376 MHZ, methanol-d₄, ppm): 8-56.47 (3F), -62.65 (3F).

### Example 3 Synthesis of Compounds 6 and 7

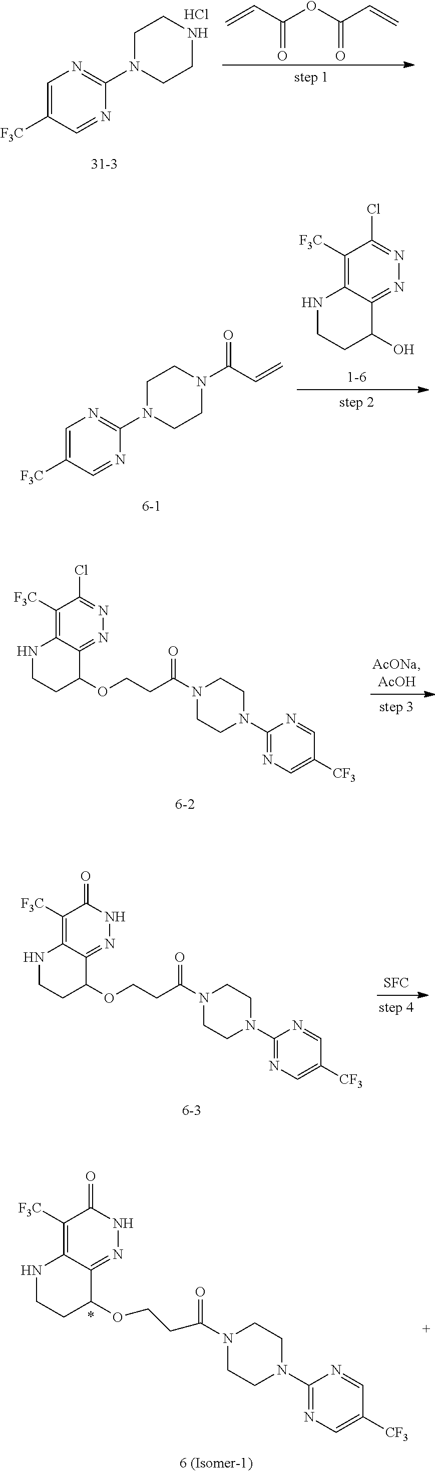

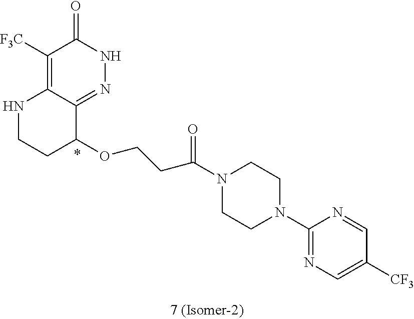 [SMILES: O=C(CCOC1CCNc2c1n[nH]c(=O)c2C(F)(F)F)N1CCN(c2ncc(C(F)(F)F)cn2)CC1]

Step 1: To a solution of 31-3 (5 g, 16.4 mmol) in dichloromethane (50 mL) was added TEA (6.6 g, 65.6 mmol) and acrylic anhydride (2.5 g, 19.7 mmol) at 0° C. The mixture was stirred at room temperature for 5 h, diluted with water and extracted with ethyl acetate. The combined organic layers were washed with brine, dried over Na₂SO₄, filtered and concentrated. The residue was purified by silica gel column chromatography (petroleum ether/ethyl acetate=3/1) to afford 6-1.

Step2: A mixture of 1-6 (200 mg, 0.79 mmol), 6-1 (1.3 g, 4.72 mmol), and Cs₂CO₃ (1.5 g, 4.72 mmol) in dioxane (10 mL) was stirred at 60° C. overnight before it was cooled and water (20 mL) was added. The mixture was extracted with ethyl acetate. The combined organic layers were washed with brine, dried over anhydrous sodium sulfate, filtered and concentrated. The residue was purified by silica gel column chromatography (ethyl acetate) to afford 6-2.

Step 3: A mixture of 6-2 (55 mg, 0.1 mmol), AcOH (61 mg, 1.0 mmol), and AcONa (84 mg, 1.0 mmol) in DMAc (6 mL) was stirred at 95° C. for 48 h before it was cooled and water (10 mL) was added. The mixture was extracted with ethyl acetate. The combined organic layers were washed with brine, dried over anhydrous sodium sulfate, filtered and concentrated. The residue was purified by prep-TLC (dichloromethane/methanol=10/1) to afford 6-3.

Step 4:6-3 was purified by SFC (column: Chiral-IG, EtOH/CO₂=40/60) to afford 6 (peak-1, 5 mg) and 7 (peak-2, 7 mg) respectively. SFC analysis of 6:99.32% ee; retention time: 7.19 min; column: Chiral-IG, EtOH/CO₂=40/60; pressure: 100 bar; flow rate: 2.0 mL/min. LCMS (ESI, m z): [M+H]⁺=522.0; ¹H NMR (400 MHZ, methanol-d₄, ppm): δ 8.59 (s, 2H), 4.39 (t, J=2.4 Hz, 1H), 3.95-3.84 (m, 6H), 3.70-3.64 (m, 4H), 3.47-3.34 (m, 2H), 2.76-2.67 (m, 2H), 2.28-2.24 (m, 1H), 1.90-1.89 (m, 1H). SFC analysis of 7:98.62% ee; retention time: 9.16 min; column: Chiral-IG, EtOH/CO₂=40/60; pressure: 100 bar; flow rate: 2.0 mL/min. LCMS (ESI, m z): [M+H]⁺=522.0; ¹H NMR (400 MHZ, methanol-d₄, ppm): δ 8.59 (s, 2H), 4.39 (t, J=2.8 Hz, 1H), 3.95-3.85 (m, 6H), 3.70-3.64 (m, 4H), 3.47-3.34 (m, 2H), 2.76-2.67 (m, 2H), 2.28-2.24 (m, 1H), 1.90-1.89 (m, 1H).

### Example 4 Synthesis of Compounds 8 and 9

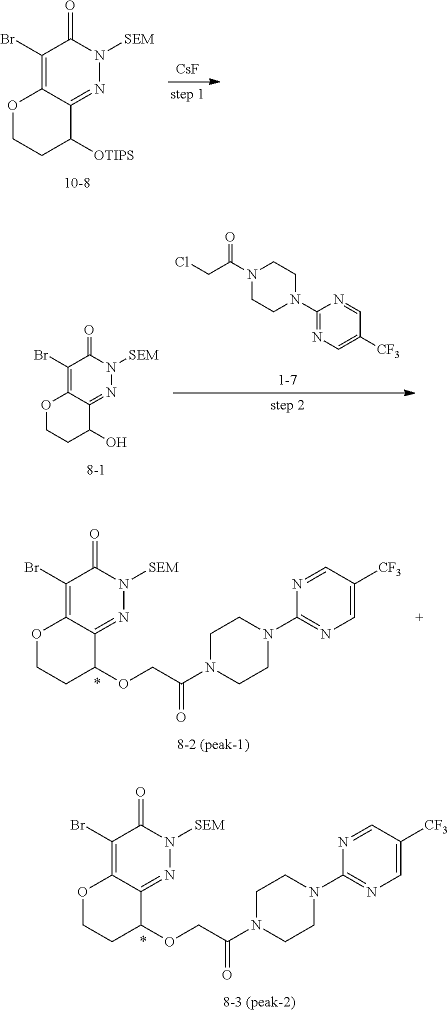

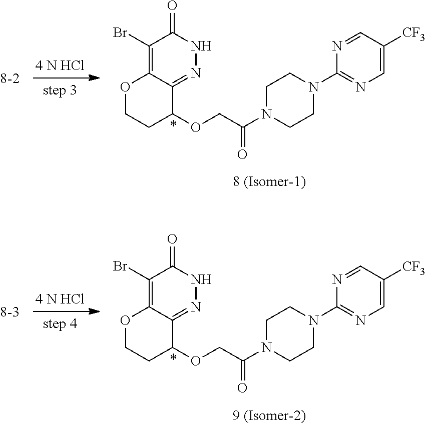 [SMILES: "4 N HCl" ; O=C(COC1CCOc2c1n[nH]c(=O)c2Br)N1CCN(c2ncc(C(F)(F)F)cn2)CC1 ; "4 N HCl" ; O=C(COC1CCOc2c1n[nH]c(=O)c2Br)N1CCN(c2ncc(C(F)(F)F)cn2)CC1]

Step 1: To a solution of 10-8 (800 mg, 1.50 mmol) in acetonitrile (20 mL) was added CsF (455 mg, 3.00 mmol). The reaction was stirred at 60° C. for 6 h. The mixture was filtered through a celite pad, and the filtrate was concentrated under vacuum. The residue was purified by silica gel column chromatography (petroleum ether/ethyl acetate=2/3) to afford 8-1.

Step 2: To a solution of 8-1 (440 mg, 1.17 mmol) in THF (20 mL) was added in portions 60% w.t NaH (16 mg, 0.4 mmol, 60% wt.) at 0° C. After the reaction mixture was stirred at 0° C. for 20 min, 1-7 (432 mg, 1.40 mmol) was added and the mixture was stirred at room temperature for 2 h. The reaction mixture was quenched with H₂O and extracted with ethyl acetate. The organic layers were combined and dried over anhydrous Na₂SO₄, filtered and concentrated. The residue was purified by silica gel column chromatography (petroleum ether to ethyl acetate) and then by chiral pre-SFC (column: REGIS (S,S) WHELK-01, MeOH (+0.1% 7.0 mol/L ammonia in MeOH)/CO₂=50/50) to afford 8-2 (peak-1, 200 mg) and 8-3 (peak-2, 150 mg).

Step 3: A mixture of 8-2 (50 mg, 0.077 mmol) in 4N HCl in dioxane (5 mL) was stirred at room temperature for 16 h. The solvent was removed under vacuum and the residue was purified by prep-HPLC (acetonitrile with 0.05% of TFA in water: 5% to 65%) to afford 8 as a 0.40 eq of TFA salt. SFC analysis: 95.96% ee; retention time: 2.86 min; column: REGIS (S,S) WHELK-01, MeOH (0.1% of DEA) in CO₂, 5% to 40%; pressure: 100 bar; flow rate: 1.5 mL/min. LCMS (ESI, m z): [M+H]⁺=519.2; ¹H NMR (400 MHZ, DMSO-d₆, ppm): δ 13.17 (s, 1H), 8.72 (s, 2H)4.51-4.50 (m, 2H), 4.50-4.48 (m, 3H), 3.86-3.73 (m, 4H), 3.60-3.45 (m, 4H), 2.30-2.28 (m, 2H). ¹⁹F NMR (376 MHz, DMSO-d₆, ppm): 8-59.32 (3F).

Step 4: A mixture of 8-3 (50 mg, 0.077 mmol) in 4N HCl in dioxane (3 mL) was stirred at room temperature for 16 h. The solvent was removed under vacuum and the residue was purified by prep-HPLC (acetonitrile with 0.05% of TFA in water: 5% to 65%) to afford 9 as a 1 eq of TFA salt. SFC analysis: 98.98% ee; retention time: 2.33 min; column: REGIS (S,S) WHELK-01, MeOH (0.1% of DEA) in CO₂, 5% to 40%; pressure: 100 bar; flow rate: 1.5 mL/min. LCMS (ESI, m z): [M+H]⁺=521.2; ¹H NMR (400 MHZ, DMSO-d₆, ppm): δ 13.17 (s, 1H), 8.72 (s, 2H)4.50-4.49 (m, 2H), 4.40-4.30 (m, 3H), 3.82-3.75 (m, 4H), 3.46-3.44 (m, 4H), 2.29-2.17 (m, 2H). ¹⁹F NMR (376 MHz, DMSO-d₆, ppm): δ-59.330 (3F).

### Example 5 Synthesis of Compounds 10 and 11

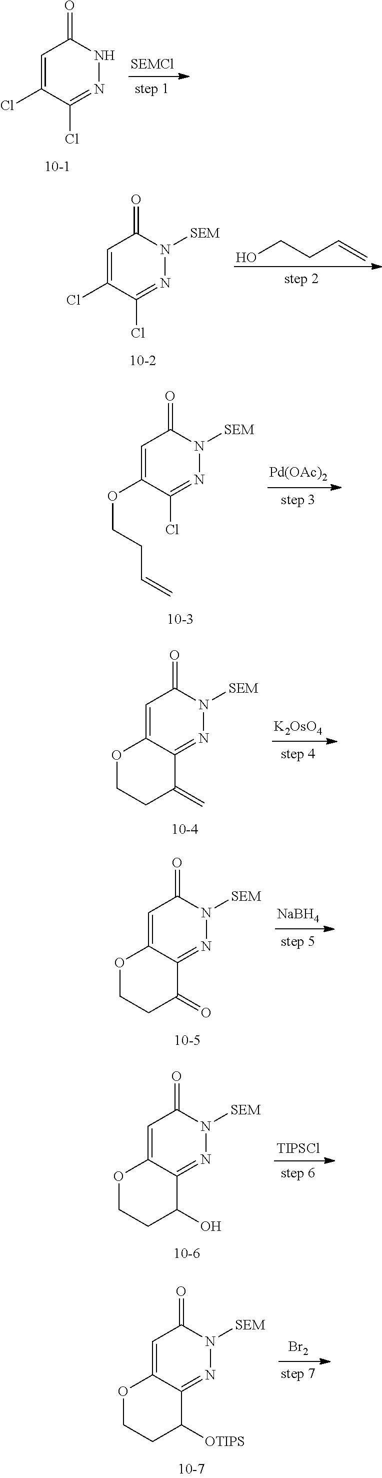

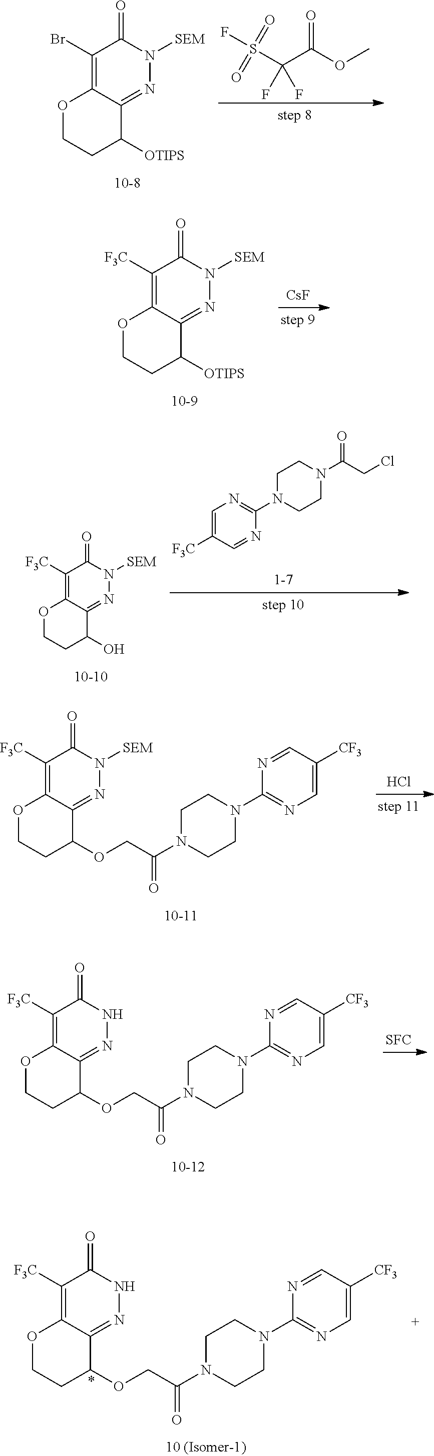

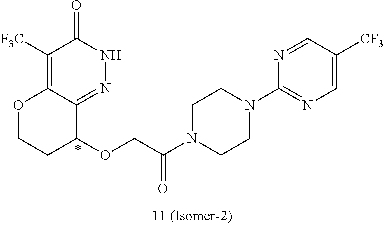 [SMILES: O=C(COC1CCOc2c1n[nH]c(=O)c2C(F)(F)F)N1CCN(c2ncc(C(F)(F)F)cn2)CC1]

Step 1: To a solution of 10-1 (15 g, 90.9 mmol) in DMF (200 mL) at 0° C. was added NaH (5.5 g, 138 mmol, 60% wt) in portions before it was stirred for 30 min and then [2-(chloromethoxy) ethyl]trimethylsilane (24 mL, 135 mmol) was added dropwise. The reaction mixture was stirred at room temperature for 2 h, quenched with H₂O and extracted with ethyl acetate. The combined organic layers were washed with H₂O, dried over anhydrous sodium sulfate, filtered and concentrated. The residue was purified by silica gel column chromatography (petroleum ether/ethyl acetate=5/1) to afford 10-2.

Step 2: To a solution of but-3-en-1-ol (6.35 g, 88 mmol) in THF (150 mL) at 0° C. was added NaH (3.52 g, 88 mmol, 60% wt) in portions. The mixture was stirred at 0° C. for 30 min, then added 10-2 (13 g, 44 mmol) was added. The mixture was stirred at room temperature for 1 h, quenched with H₂O and extracted with ethyl acetate. The organic layers were combined, dried over anhydrous sodium sulfate, filtered and concentrated. The residue was purified by silica gel column chromatography (petroleum ether/ethyl acetate=3/1) to afford 10-3.

Step 3: To a solution of 10-3 (11 g, 33.3 mmol) in DMF (200 mL) was added Pd(OAc)₂ (2.3 g, 10 mmol), Xantphos (7.79 g, 13.3 mmol), and TEA (18.5 mL, 132 mmol). The mixture was stirred at 105° C. under N₂ for 16 h. The mixture was cooled, diluted with water and extracted with ethyl acetate. The combined organic layers were dried over anhydrous sodium sulfate, filtered and concentrated. The residue was purified by silica gel column chromatography (petroleum ether/ethyl acetate=3/1) to afford 10-4.

Step 4: To a solution of 10-4 (5.4 g, 18.3 mmol) in a mixture of THF (100 mL) and H₂O (100 mL) was added dipotassium dioxoosmiumbis (olate) dihydrate (67.6 mg, 0.18 mmol) and sodium periodate (15.7 g, 73.4 mmol). The reaction mixture was stirred at room temperature for 3 h, then diluted with H₂O and extracted with ethyl acetate. The combined organic layers were dried over anhydrous sodium sulfate, filtered and concentrated. The residue was purified by silica gel column chromatography (petroleum ether/ethyl acetate=3/2) to afford 10-5.

Step 5: To a solution of 10-5 (4.15 g, 14 mmol) in MeOH (100 mL) at 0° C. was added NaBH₄ (0.8 g, 21 mmol) in portions. The reaction mixture was stirred at room temperature for 1 h, then quenched with H₂O and extracted with ethyl acetate. The combined organic layers were dried over anhydrous sodium sulfate, filtered and concentrated to afford 10-6 which was used directly in the next step without purification.

Step 6: To a solution of 10-6 (4 g, 13.4 mmol) in DMF (100 mL) was added imidazole (2.7 g, 40.2 mmol) and triisopropylsilyl chloride (6 mL, 28.1 mmol). The reaction mixture was stirred at 60° C. for 16 h. The mixture was cooled, diluted with water and extracted with ethyl acetate. The combined organic layers were dried over anhydrous sodium sulfate, filtered and concentrated. The residue was purified by silica gel column chromatography (petroleum ether/ethyl acetate=5/2) to afford 10-7.

Step 7: To a solution of 10-7 (2 g, 4.4 mmol) and KOAc (648 mg, 6.6 mmol) in AcOH (30 mL) at 0° C. was added Br₂ (0.35 mL, 6.8 mmol) dropwise. The mixture was stirred at room temperature for 16 h, quenched with H₂O (40 mL) and saturated Na₂SO₃ aqueous solution (5 mL), and extracted with ethyl acetate. The combined organic layers were washed with brine, dried over anhydrous sodium sulfate, filtered and concentrated. The residue was purified by silica gel column chromatography (petroleum ether/ethyl acetate=4/1) to afford 10-8.

Step 8: To a solution of 10-8 (1.0 g, 1.874 mmol) in DMF (20 mL) was added methyl 2,2-difluoro-2-(fluorosulfonyl) acetate (1.08 g, 5.62 mmol) and Cul (357 mg, 1.9 mmol). The reaction mixture was stirred at 110° C. under N₂ for 3 h. The mixture was cooled, diluted with water and extracted with ethyl acetate. The combined organic layers were dried over anhydrous sodium sulfate, filtered and concentrated to afford 10-9 which was used directly in the next step without purification.

Step 9: To a solution of 10-9 (460 mg, 0.88 mmol) in acetonitrile (15 mL) was added cesium fluoride (267 mg, 1.76 mmol). The reaction mixture was stirred at 60° C. for 2 h. The mixture was cooled, filtered and the filtrate was concentrated. The residue was purified by silica gel column chromatography (petroleum ether/ethyl acetate=1/1) to afford 10-10.

Step 10: To a solution of 10-10 (100 mg, 0.27 mmol) and 1-7 (101 mg, 0.33 mmol) in THF (10 mL) at 0° C. was added NaH (33 mg, 0.825 mmol, 60% dispersion in mineral oil) in portions. The mixture was stirred at room temperature for 2 h, then quenched with H₂O and extracted with ethyl acetate. The combined organic layers were dried over anhydrous sodium sulfate, filtered and concentrated. The residue was purified by silica gel column chromatography (petroleum ether to ethyl acetate) to afford 10-11.

Step 11:10-11 (115 mg, 0.18 mmol) was dissolved in a solution of HCl in dioxane (4 M, 5 mL). The mixture was stirred at room temperature for 4 h. The solvent was removed under vacuum. The residue was purified by prep-HPLC (acetonitrile with 0.05% of TFA in water: 5% to 42%) to afford 10-12. Compound 10-12 (85 mg) was purified by SFC (column: DAICEL CHIRALCEL®OZ, MeOH (+0.1% 7.0 mol/L ammonia in MeOH)/CO₂=60/40) to afford 10 (15.5 mg) and 11 (18 mg), respectively. SFC analysis of 10: 96% ee; retention time: 3.98 min; column: DAICEL CHIRALCEL®OZ, MeOH (0.1% of DEA) in CO₂, 5% to 40%; pressure: 100 bar; flow rate: 1.5 mL/min. LCMS (ESI, m/z): [M+H]=509.2; ¹H NMR (400 MHZ, DMSO-d₆) δ 13.24 (s, 1H), 8.72 (s, 2H), 4.55-4.38 (m, 3H), 4.36 (s, 2H), 3.99-3.62 (m, 4H), 3.58-3.38 (m, 4H), 2.35-2.12 (m, 2H). SFC analysis of 11:99. 36% ee; retention time: 4.63 min; column: DAICEL CHIRALCEL®OZ, MeOH (0.1% of DEA) in CO₂, 5% to 40%; pressure: 100 bar; flow rate: 1.5 mL/min. LCMS (ESI, m/z): [M+H]⁺=509.2; ¹H NMR (400 MHZ, DMSO-d₆) δ 13.25 (s, 1H), 8.72 (s, 2H), 4.56-4.39 (m, 3H), 4.41 (s, 2H), 3.92-3.72 (m, 4H), 3.60-3.37 (m, 4H), 2.32-2.12 (m, 2H).

### Example 6 Synthesis of Compound 12

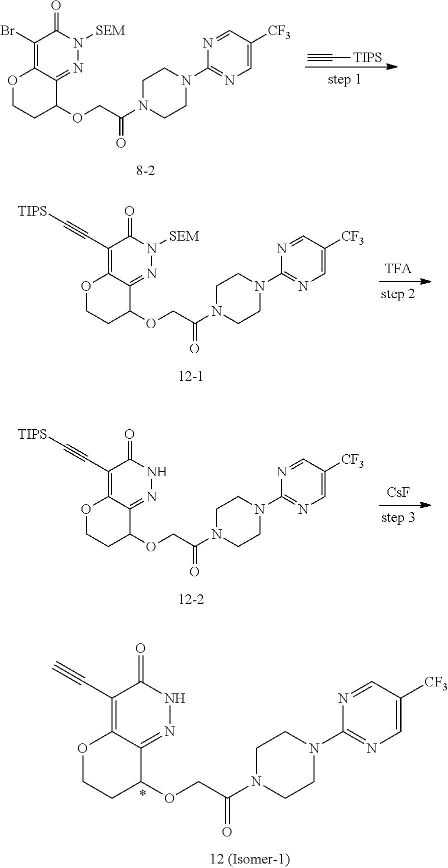

Step 1: A mixture of 8-2 (60 mg, 0.094 mmol), Cul (9 mg, 0.047 mmol), ethynyltriisopropyl-silane (52 mg, 0.28 mmol) and Pd(PPh₃)₄ (109 mg, 0.094 mmol) in DIPEA (1 mL) was flushed with nitrogen gas for 1 min and then stirred at 90° C. under microwave condition for 1 h. The mixture was cooled, diluted with water and extracted with ethyl acetate. The combined organic layers were dried over anhydrous sodium sulfate, filtered and concentrated. The residue was purified by silica gel column chromatography (dichloromethane/ethyl acetate=1/1) to afford 12-1.

Step 2: To a solution of 12-1 (60 mg, 0.080 mmol) in dichloromethane (2 mL) was added TFA (137 mg, 1.20 mmol). The mixture was stirred at room temperature for 1 h. The mixture was basified with aq. NaHCO₃ solution (1 M) and extracted with ethyl acetate. The combined organic layers were washed with brine, dried over anhydrous Na₂SO₄, filtered, and concentrated. The crude product was purified by silica gel column chromatography (dichloromethane/ethyl acetate=1/1) to afford 12-2.

Step 3: To a solution of 12-2 (40 mg, 0.064 mmol) in acetonitrile (5 mL) was added CsF (78 mg, 0.52 mmol). The mixture was stirred at 60° C. for 1 h. The mixture was cooled, diluted with water and extracted with ethyl acetate. The combined organic layers were dried over anhydrous sodium sulfate, filtered and concentrated. The residue was purified by prep-HPLC (acetonitrile with 0.05% of TFA in water: 5% to 65%) to afford 12 as a 0.76 eq of TFA salt, LCMS (ESI, m z): [M+H]⁺=464.8; ¹H NMR (400 MHZ, DMSO-d₆, ppm): δ 13.04 (s, 1H), 8.72 (s, 2H), 4.60 (s, 1H), 4.45-4.42 (m, 2H), 4.36-4.34 (m, 3H), 3.85-3.82 (m, 4H), 3.73-3.51 (m, 4H), 2.40-2.27 (m, 1H), 2.15-2.14 (m, 1H). ¹⁹F NMR (376 MHz, DMSO-d₆, ppm): δ-59.32 (3F).

### Example 7 Synthesis of Compounds 13 and 14

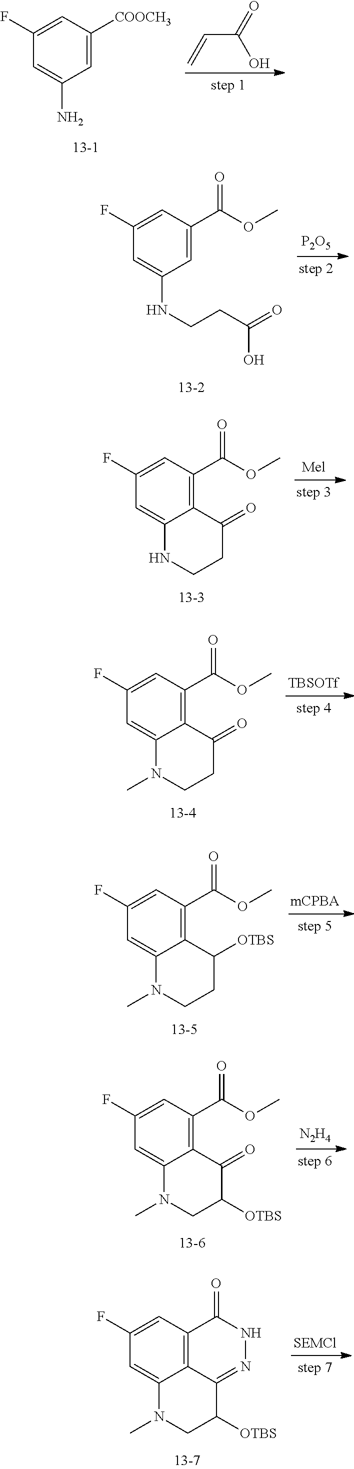

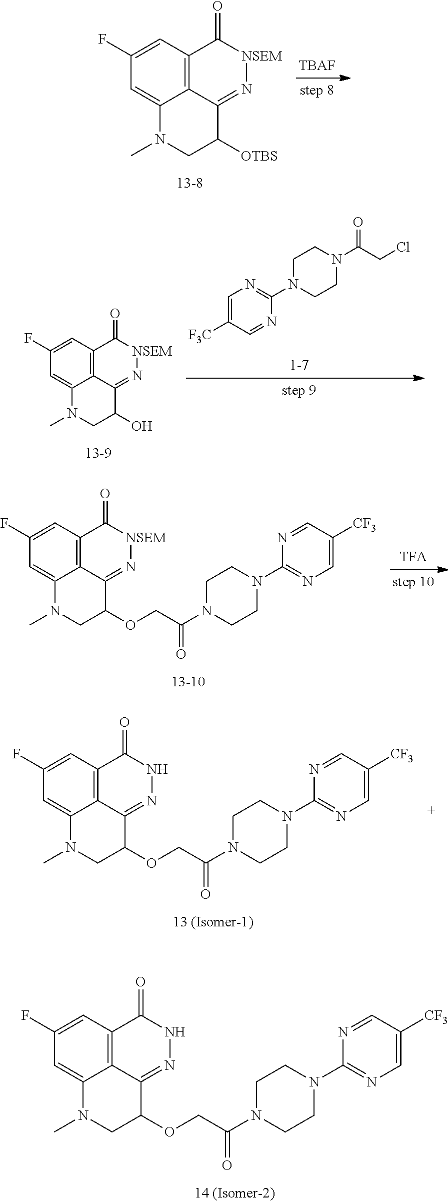

Step 1: To a solution of 13-1 (9.5 g, 56 mmol) in AcOH (100 mL) was added acrylic acid (8.09 g, 112 mmol) and H₂SO₄ (55 mg, 0.56 mmol, 30 uL) at 0° C. The resulting solution was stirred at 100° C. for 16 h. Then cooled and concentrated. The pH of the residue was adjusted to 6 and extracted with dichloromethane. The organic layer was concentrated. The residue was purified by silica gel column chromatography (petroleum ether/ethyl acetate=2/1) to afford 13-2.

Step 2: P₂O₅ (14.71 g, 104 mmol) was added to methanesulfonic acid (100 mL) under N₂. The solution was stirred at 80° C. for 1 h before the mixture was cooled, then was added 13-2 (5 g, 20.7 mmol). The reaction mixture was stirred at 55° C. for 16 h. After cooling down to 0° C., 1 M NaOH was added to adjust to pH=6 and the solution was extracted with dichloromethane. The combined organic layers were concentrated. The residue was purified by silica gel column chromatography (petroleum ether/ethyl acetate=2/1) to afford 13-3.

Step 3: To a solution of 13-3 (1.2 g, 5.38 mmol) in DMF (60 mL) was added K₂CO₃ (3.71 g, 26.88 mmol) and Mel (7.64 g, 53.8 mmol) under N₂. The solution was stirred at 55° C. for 16 h. After cooling down to room temperature, H₂O was added and the mixture was extracted with ethyl acetate. The combined organic layers were dried over anhydrous sodium sulfate, filtered and concentrated. The residue was purified by silica gel column chromatography (petroleum ether/ethyl acetate=3/1) to afford 13-4.

Step 4: A solution of 13-4 (600 mg, 2.53 mmol) in THF (10 mL) was purged with N₂ for 3 times, cooled to −70° C., and treated with LiHMDS (1.27 g, 7.59 mmol). The mixture was stirred at −70° C. for 1 h, then added TBSOTf (1.34 g, 5.06 mmol) slowly. The reaction mixture was stirred at −70° C. for 30 min, quenched with saturated aqueous NH₄Cl solution, and extracted with ethyl acetate. The combined organic layers were dried over anhydrous Na₂SO₄, filtered and concentrated to afford 13-5, which was used directly in the next step without further purification.

Step 5: To a solution of 13-5 (800 mg, 2.28 mmol) in dichloromethane (10 mL) was added mCPBA (786 mg, 4.55 mmol), and the mixture was stirred at room temperature overnight. Water was added, and the reaction mixture was extracted with dichloromethane. The combined organic layer was dried over Na₂SO₄, filtered and concentrated. The residue was purified by silica gel column chromatography (petroleum ether/ethyl acetate=10/1) to afford 13-6.

Step 6: To a solution of 13-6 (370 mg, 1.01 mmol) in MeOH (10 mL) was added N₂H₄ (32.26 mg, 1.01 mmol), and the mixture was stirred at room temperature overnight. The reaction mixture was concentrated and the residue was purified by silica gel column chromatography (petroleum ether/ethyl acetate=5/1) to afford 13-7.

Step 7: A solution of 13-7 (300 mg, 0.86 mmol) in DMF (10 mL) was cooled to 0° C., then treated with NaH (41 mg, 1.72 mmol). The mixture was stirred at room temperature for 1 h. Then SEMCI (172 mg, 1.03 mmol) was added. After stirring at room temperature for 1 h, water was added, and the reaction mixture was extracted with ethyl acetate. The combined organic layers were washed with water, dried over anhydrous Na₂SO₄, filtered and concentrated. The mixture was concentrated and the residue was purified by silica gel column chromatography (petroleum ether/ethyl acetate=10/1) to afford 13-8.

Step 8: To a solution of 13-8 (280 mg, 0.58 mmol) in THF (10 mL) was added TBAF (16 mg, 0.58 mmol), and the mixture was stirred at room temperature overnight. Water was added and the mixture was extracted with ethyl acetate. The combined organic layers were dried over anhydrous Na₂SO₄, filtered and concentrated. The residue was purified by silica gel column chromatography (petroleum ether/ethyl acetate=1/1) to afford 13-9.

Step 9: A solution of 13-9 (80 mg, 0.22 mmol) in DMF (5 mL) was cooled to 0° C. and treated with NaH (11 mg, 0.44 mmol). The mixture was stirred at room temperature for 30 min, 1-7 (68 mg, 0.22 mmol) was then added. After stirring at room temperature for 16 h. Water was added to quench the reaction. The mixture was extracted with ethyl acetate. The combined organic layers were washed with water, dried over anhydrous Na₂SO₄, filtered and concentrated by. The residue was purified by prep-TLC (dichloromethane/methanol=30/1) to afford 13-10.

Step 10: To a solution of 13-10 (50 mg, 0.078 mmol) in dichloromethane (1 mL) was added TFA (1 mL). The mixture was stirred at room temperature for 1 h and concentrated. The crude product was dissolved in 3 mL of THF. Ammonia was added to adjust the mixture to pH=8. The mixture was stirred at room temperature for 1 h, concentrated, and the residue was purified by prep-HPLC (acetonitrile with 0.1% of FA in water, 5% to 95%) and SFC (column: REGIS (S,S) WHELK-01, MeOH (+0.1% 7.0 mol/L ammonia in MeOH)/CO₂=50/50) to afford 13 (peak-1, 9 mg) and 14 (peak-2, 4 mg). SFC analysis of 13:99.70% ee; retention time: 1.92 min; column: REGIS (S,S) WHELK-01, MeOH (0.1% of DEA) in CO₂, 40%; pressure: 100 bar; flow rate: 1.5 mL/min. LC-MS: (ES, m/z): [M+H]⁺=508.3; ¹H NMR (400 MHZ, methanol-d₄): δ 8.58 (d, J=0.6 Hz, 2H), 7.13 (dd, J=8.9, 2.3 Hz, 1H), 6.80 (dd, J=12.1, 2.3 Hz, 1H), 4.53 (t, J=2.6 Hz, 1H), 4.40 (dd, J=32.5, 14.2 Hz, 2H), 3.90-3.80 (m, 5H), 3.69-3.43 (m, 5H), 3.10 (s, 3H). SFC analysis of 14:97. 24% ee; retention time: 2.29 min; column: REGIS (S,S) WHELK-01, MeOH (0.1% of DEA) in CO₂, 40%; pressure: 100 bar; flow rate: 1.5 mL/min. LC-MS: (ES, m/z): [M+H]⁺=508.3; ¹H NMR (400 MHZ, methanol-d₄): δ 8.58 (s, 2H), 7.14 (dd, J=8.8, 2.3 Hz, 1H), 6.80 (dd, J=12.1, 2.3 Hz, 1H), 4.53 (t, J=2.6 Hz, 1H), 4.40 (dd, J=32.5, 14.2 Hz, 2H), 3.90-3.79 (m, 5H), 3.59 (ddd, J=51.4, 23.4, 2.1 Hz, 5H), 3.10 (s, 3H).

### Example 8 Synthesis of Compounds 15 and 17

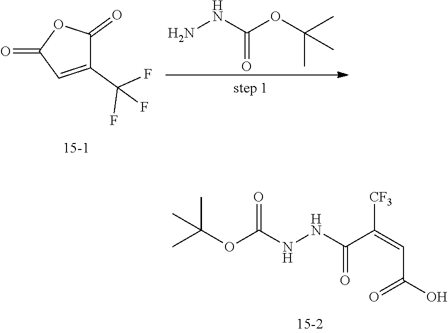

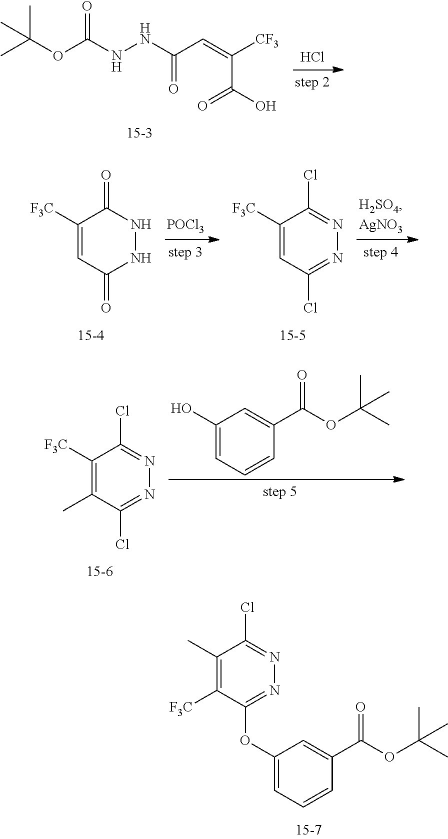

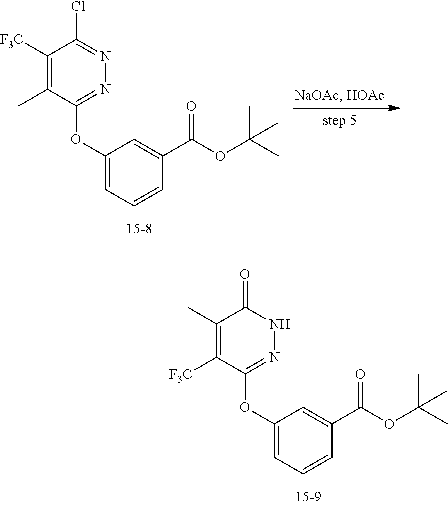

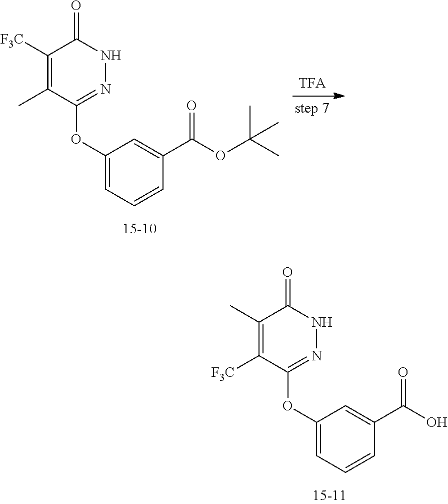

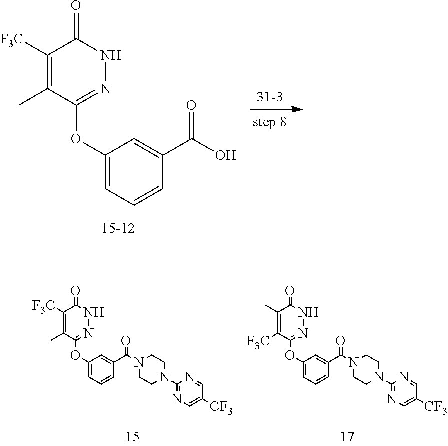 [SMILES: Cc1c(Oc2cccc(C(=O)O)c2)n[nH]c(=O)c1C(F)(F)F ; Cc1c(Oc2cccc(C(=O)N3CCN(c4ncc(C(F)(F)F)cn4)CC3)c2)n[nH]c(=O)c1C(F)(F)F ; Cc1c(C(F)(F)F)c(Oc2cccc(C(=O)N3CCN(c4ncc(C(F)(F)F)cn4)CC3)c2)n[nH]c1=O]

Step 1: To a solution of 15-1 (25 g, 0.15 mol) in ethyl ether (470 mL) was added dropwise a solution of tert-butoxycarbonyl hydrazide (20 g, 0.15 mol) in ethyl ether (470 mL) at 0° C. The resulting mixture was stirred at room temperature for 1 h. Solvent was evaporated under reduced pressure to afford a mixture of 15-2 and 15-3 which was used in the next step directly without further purification.

Step 2: A solution of the mixture of 15-2 and 15-3 (crude, 0.15 mol) in 1.25 M HCl/MeOH solution (340 mL, 0.43 mol) was stirred at 50° C. for 3 h. After being concentrated, water (150 mL) was added to the residue and the resulting slurry was filtered. The filter cake was dried under vacuum to afford 15-4.

Step 3: A solution of 15-4 (5 g, 27.78 mmol) in phosphorus oxychloride (50 mL) was stirred at 120° C. for 1.5 h under microwave condition. Solvent was evaporated under reduced pressure and the crude material was purified by silica gel column chromatography (petroleum ether/methyl tert-butyl ether=3/1) to afford 15-5.

Step 4: Under nitrogen, to a solution of concentrated H₂SO₄ (426 mg, 4.30 mmol) in water (27 mL) were added 15-5 (1.85 g, 8.52 mmol), AgNO₃ (290 mg, 1.70 mmol) and HOAc (565 mg, 9.42 mmol). The reaction mixture was heated to 55° C. and a solution of (NH₄)₂S₂O₈ (2.91 g, 12.76 mmol) in water (9 mL) was added dropwise over 1 h. The resulting mixture was stirred at 55° C. for 1 h. The mixture was cooled, neutralized to pH=7 with diluted aqueous ammonia solution and extracted with methyl tert-butyl ether. The combined organic layers were dried over sodium sulfate, filtered and concentrated. The residue was purified by column chromatography on silica gel (petroleum ether/methyl tert-butyl ether=3/1) to afford 15-6.

Step 5: A mixture of 15-6 (770 mg, 3.41 mmol), 3-hydroxybenzoic acid tert-butyl ester (794 mg, 4.09 mmol) and K₂CO₃ (940 mg, 6.81 mmol) in DMF (7 mL) was stirred at 40° C. for 3 h. After being quenched with saturated aqueous NH₄Cl solution (100 mL), the mixture was extracted with ethyl acetate. The combined organic layers were dried over sodium sulfate, filtered and concentrated. The residue was purified by prep-HPLC (acetonitrile with 0.05% of TFA in water: 5% to 95%) to afford a mixture of 15-7 and 15-8, which was used in the next step directly without further purification.

Step 6: The mixture of 15-7 and 15-8 (988 mg, 2.55 mmol) and NaOAc (418 mg, 5.10 mmol) in HOAc (10 mL) was stirred at 120° C. for 4 h. The mixture was cooled, diluted with saturated aqueous NH₄Cl solution and extracted with ethyl acetate. The combined organic layers were dried over sodium sulfate, filtered and concentrated to afford a mixture of 15-9 and 15-10 which was used directly in next step without further purification.

Step 7: The mixture of 15-9 and 15-10 (crude, 2.54 mmol) in TFA (10 mL) was stirred at room temperature for 16 h. The mixture was concentrated and the residue was purified by reverse phase HPLC (acetonitrile with 0.05% of TFA in water, 5% to 95%) to afford a mixture of 15-11 and 15-12, which was used in the next step without further purification.

Step 8: Under nitrogen, to a mixture of 15-11 and 15-12 (730 mg, 2.17 mmol), 31-3 (582 mg, 2.17 mmol) and DIPEA (840 mg, 6.51 mmol) in DMF (14 mL) was added HATU (618 mg, 1.63 mmol) portion wise at 0° C. The resulting mixture was stirred at this temperature for 1.5 h. The mixture was diluted with water and extracted with ethyl acetate. The combined organic layers were dried over sodium sulfate, filtered and concentrated. The residue was purified by prep-HPLC (acetonitrile with 0.05% of TFA in water, 5% to 95%) and SFC (column: DAICEL CHIRALCEL®OZ, MeOH (+0.1% 7.0 M ammonia in MeOH)/CO₂) to afford 15 (51.5 mg) and 17 (221.4 mg) respectively. 15, LCMS (ESI, m z): [M+H]=529.2. ¹H NMR (400 MHZ, CDCl₃, ppm): δ 11.28 (s, 1H), 8.50 (s, 2H), 7.49 (t,J=7.9 Hz, 1H), 7.33 (d, J=7.6 Hz, 1H), 7.26-7.24 (m, 1H), 7.23-7.19 (m, 1H), 4.10-3.75 (m, 6H), 3.70-3.45 (m, 2H), 2.53-2.50 (m, 3H). 17, LCMS (ESI, m z): [M+H]=529.2. ¹H NMR (400 MHZ, CDCl₃, ppm): δ 8.50 (s, 2H), 7.46 (t, J=7.9 Hz, 1H), 7.31 (d, J=7.6 Hz, 1H), 7.27-7.26 (m, 1H), 7.25-7.21 (m, 1H), 4.05-3.70 (m, 6H), 3.65-3.40 (m, 2H), 2.45 (d, J=3.2 Hz, 3H).

### Example 9 Synthesis of Compound 19

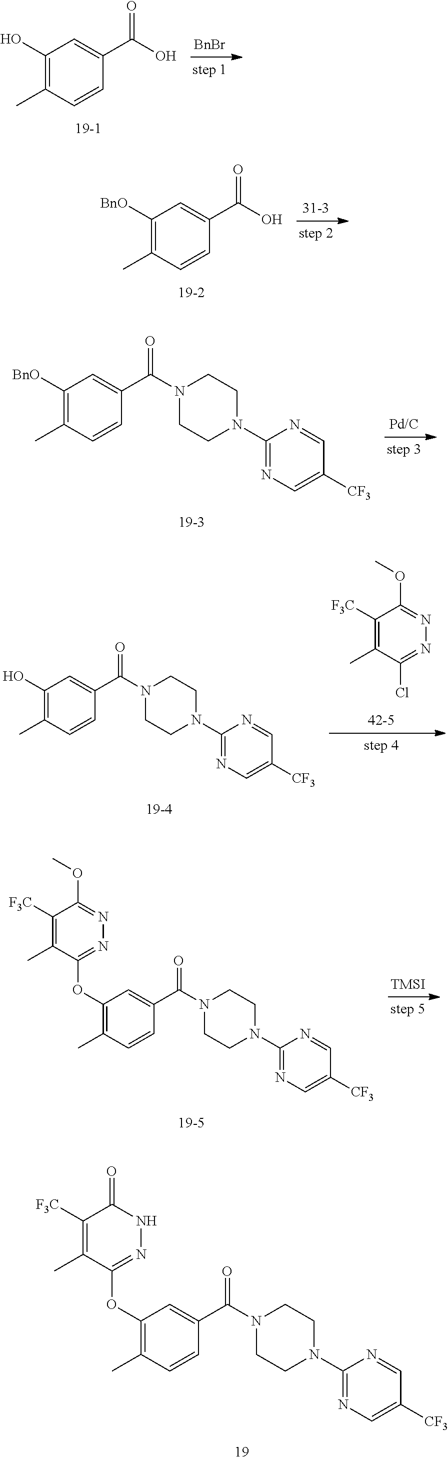

Step 1: To a suspension of 19-1 (10.0 g, 65.7 mmol) in DMF (100 mL) was added BnBr (23.6 g, 138.0 mmol) and K₂CO₃ (36.3 g, 262.8 mmol). The resulting mixture was stirred at room temperature overnight, filtered and washed with ethyl acetate. The filtrate was washed with water and brine, dried over Na₂SO₄ and concentrated under vacuum. The crude residue was dissolved in MeOH (50 mL) and water (50 mL), added NaOH (8.1 g, 203.7 mmol, 3.1 eq), then stirred at room temperature for 3 h. The mixture was concentrated to remove MeOH. The aqueous layer was adjusted to pH 2-3 with 3N HCl and extracted with ethyl acetate. The organic layers were combined, dried over Na₂SO₄, filtered and concentrated to afford 19-2, which was used directly in the next step without further purification.

Step 2: To a solution of 19-2 (1.0 g, 4.1 mmol) and 31-3 (1.6 g, 5.4 mmol) in DMF (10 mL) was added HOBT (670 mg, 5.0 mmol), EDCI (950 mg, 5.0 mmol) and TEA (1.5 g, 14.9 mmol). The mixture was stirred at room temperature for 3 h. The mixture was diluted with water and extracted with ethyl acetate. The combined organic layers were dried over sodium sulfate, filtered and concentrated. The residue was purified by silica gel column chromatography (petroleum ether/methyl tert-butyl ether=3/1) to afford 19-3.

Step 3: To a solution of 19-3 (1.5 g, 3.3 mmol) in EtOH (30 mL) was added Pd/C (150 mg, 10% w/w). The mixture was stirred at room temperature under the atmosphere of H₂ for 4 h, filtered through a pad of Celite and washed with ethyl acetate. The filtrate was concentrated to afford 19-4, which was used in the next step without further purification.

Step 4: Under N₂ to a mixture of 19-4 (194 mg, 0.53 mmol) and 42-5 (100 mg, 0.44 mmol) in dioxane (5 mL) were added Pd₂ (dba)₃ (81 mg, 0.09 mmol), MeatBuXphos (52 mg, 0.18 mmol) and Cs₂CO₃ (288 mg, 0.88 mmol). The mixture was stirred at 95° C. for 3 h. The mixture was diluted with water and extracted with ethyl acetate. The combined organic layers were dried over sodium sulfate, filtered and concentrated. The residue was purified by silica gel column chromatography (petroleum ether/ethyl acetate=8/1) to afford 19-5.

Step 5: To a solution of 19-5 (60 mg, 0.11 mmol) in MeCN(3 mL) was added TMSI (43 mg, 0.22 mmol). The mixture was stirred at room temperature for 1 h. The mixture was diluted with water and extracted with ethyl acetate. The combined organic layers were dried over sodium sulfate, filtered and concentrated. The residue was purified by prep-HPLC (acetonitrile with 0.1% FA in water, 40% to 65%) to afford 19. LCMS (ESI, m z): [M+H]⁺=543.0; ¹H NMR (400 MHZ, methanol-d₄, ppm): δ 8.60 (s, 2H), 7.43 (d, J=7.2 Hz, 1H), 7.30-7.28 (m, 2H), 4.02-3.97 (m, 4H), 3.83-3.81 (m, 2H), 3.60-3.57 (m, 2H), 2.57-2.55 (m, 3H), 2.27 (s, 3H). ¹⁹F NMR (376 MHZ, methanol-d₄, ppm): δ-60.66 (3F), -62.67 (3F).

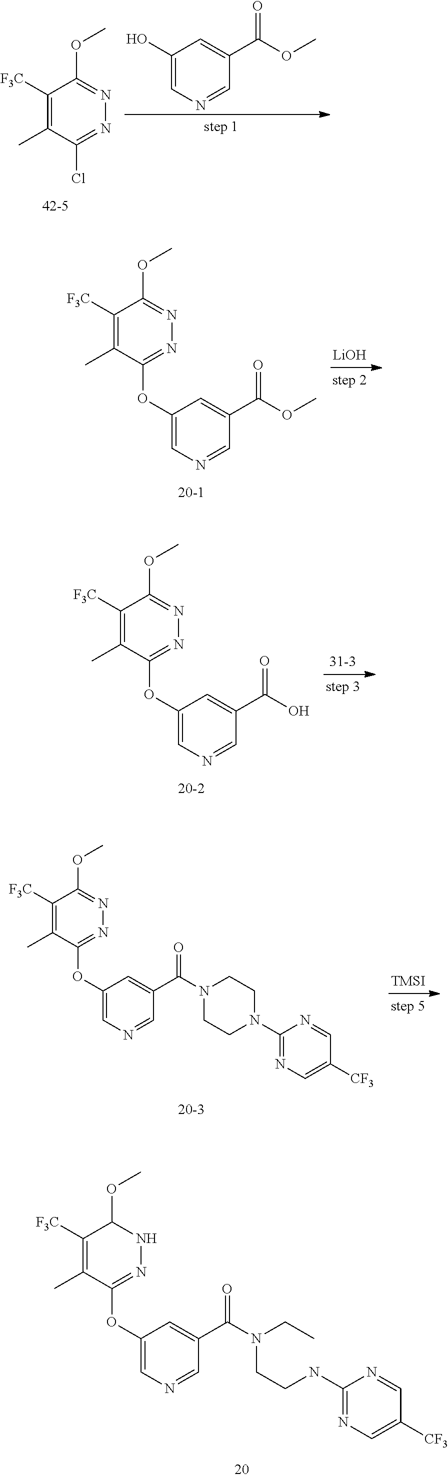

Step 1: To a solution of 42-5 (290 mg, 1.28 mmol) and methyl 5-hydroxynicotinate (235 mg, 1.54 mmol) in dioxane (3 mL) were added Pd₂ (dba)₃ (117 mg, 0.13 mmol), Meat-BuXPhos (123 mg, 0.26 mmol) and Cs₂CO₃ (834 mg, 2.56 mmol). The resulting mixture was degassed under nitrogen for 10 min, then stirred for 3.5 h at 90° C. The mixture was cooled, concentrated and the residue was purified by silica gel column chromatography (petroleum ether/ethyl acetate=5/1) to afford 20-1.

Step 2: To a solution of 20-1 (200 mg, 0.58 mmol) in methanol (3 mL) and water (3 mL) was added LiOH (73 mg, 1.75 mmol). The reaction mixture was stirred at room temperature for 5 h, acidified to pH=6 with 1 N HCl, and extracted with ethyl acetate. The combined organic layers were washed with brine, dried over Na₂SO₄, filtered and concentrated. The residue was purified by silica gel column chromatography (petroleum ether to ethyl acetate) to afford 20-2.

Step 3: To a solution of 20-2 (110 mg, 0.33 mmol) in DMF (3 mL) were added DIPEA (0.17 mL, 1.00 mmol), HATU (140 mg, 0.37 mmol) and 31-3 (99 mg, 0.37 mmol). The reaction mixture was stirred for another 30 min at room temperature. The mixture was purified by reverse phase HPLC (acetonitrile with 0.05% of TFA in water, 5% to 95%) to afford 20-3.

Step 4: To a solution of 20-3 (80 mg, 0.15 mmol) in acetonitrile (10 mL) was added TMSI (88 mg, 0.44 mmol). The reaction mixture was stirred at 70° C. for 1.5 h. The mixture was diluted with water and extracted with ethyl acetate. The combined organic layers were dried over sodium sulfate, filtered and concentrated. The residue was purified by prep-HPLC (acetonitrile with 0.05% of TFA in water: 5% to 52%) to afford 20 as a 0.52 eq of TFA salt, LCMS (ESI, m z): [M+H]⁺=530.2; ¹H NMR (400 MHZ, DMSO-d₆, ppm): δ 12.76 (s, 1H), 8.72 (s, 2H), 8.62 (d, J=2.8 Hz, 1H), 8.54 (d, J=2.4 Hz, 1H), 7.88 (t, J=2.0 Hz, 1H), 3.92-3.89 (m, 4H), 3.74-3.65 (m, 2H), 3.50-3.42 (m, 2H), 2.41 (t, J=2.8 Hz, 3H). 19F NMR (376 MHz, DMSO-d₆, ppm): δ-58.49, −59.34.

### Example 11 Synthesis of Compound 27

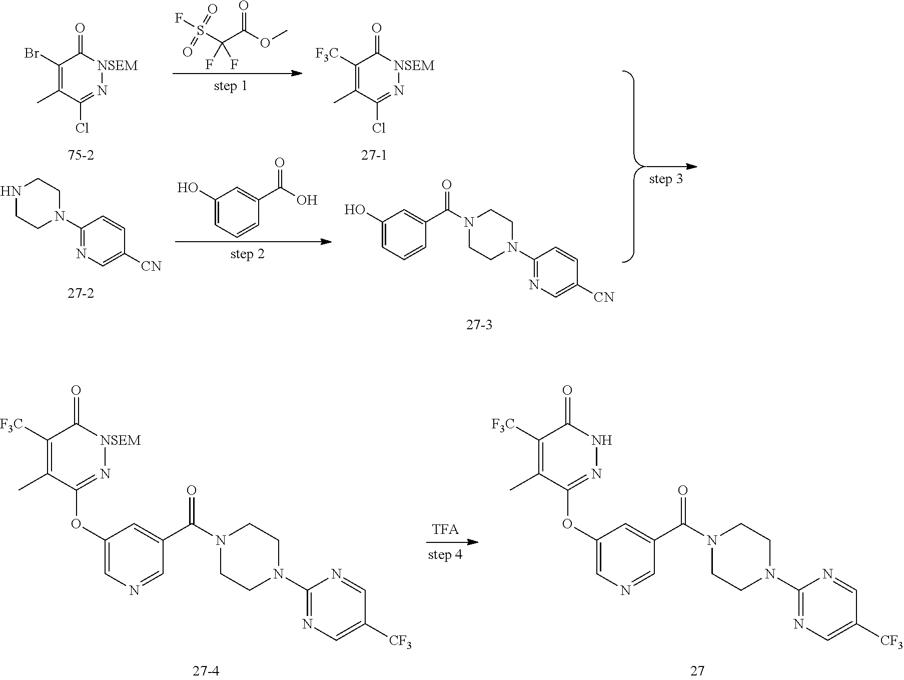

Step 1: Compound 27-1 was prepared from compound 75-2 following the procedure for the synthesis of compound 36-3 in example 14.

Step 2: To a solution of 3-hydroxybenzoic acid (300 mg, 2.17 mmol) and 27-2 (409 mg, 2.17 mmol) in dichloromethane (10 mL) was added DIEA (281 mg, 2.17 mmol, 378 uL) followed by HATU (819 mg, 2.17 mmol). The mixture was stirred at room temperature for 2 h. Water was added and the mixture was extracted with dichloromethane. The organic layer was dried over Na₂SO₄, filtered and concentrated. The crude product was purified by silica gel column chromatography (petroleum ether/ethyl acetate=1/1) to afford 27-3.

Step 3: To a solution of 27-3 (270 mg, 0.88 mmol) and 27-1 (300 mg, 0.88 mmol) in toluene (10 mL) was added RockPhos (4.10 mg, 0.88 mmol), Pd₂ (dba)₃ (8.0 mg, 0.88 mmol) and K₃PO₄ (186 mg, 0.88 mmol). The mixture was purged with N₂ for 3 times and then stirred at 100° C. overnight. Water was added and the mixture was extracted with ethyl acetate. The organic layer was dried over Na₂SO₄, filtered and concentrated. The crude product was purified by silica gel column chromatography (petroleum ether/ethyl acetate=1/1) to afford 27-4.

Step 4: To a solution of 27-4 (40 mg, 0.065 mmol) in dichloromethane (1 mL) was added TFA (1 mL). The mixture was stirred at room temperature for 1 h. The mixture was concentrated. The crude product was dissolved in THF (1 mL) and ammonia was added (1 mL). The mixture was stirred at room temperature for 1 h and concentrated. The crude was purified by prep-HPLC (acetonitrile with 0.1% of FA in water, 5% to 95%) to afford 27. LCMS: (ESI, m z): [M+H]⁺=485.1. ¹H NMR (400 MHZ, DMSO-d₆, ppm): δ 8.42 (d, J=1.9 Hz, 1H), 7.76 (dd, J=9.1, 2.3 Hz, 1H), 7.54 (t, J=8.0 Hz, 1H), 7.41-7.27 (m, 3H), 6.88 (d, J=9.1 Hz, 1H), 3.96-3.52 (m, 8H), 2.52 (s, 3H).

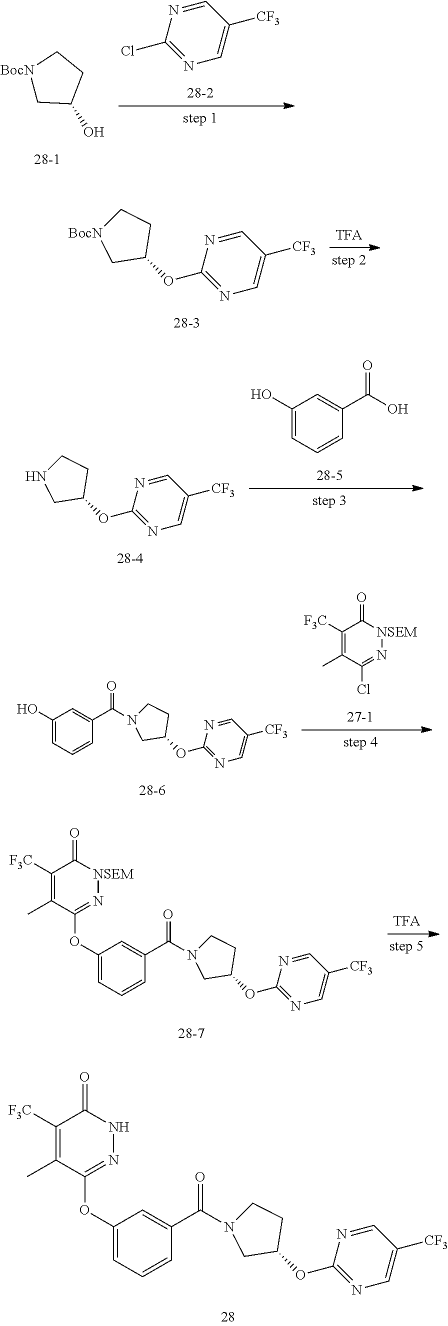

Step 1: To a solution of 28-1 (700 mg, 3.74 mmol) in DMF (10 mL) was added NaH (60% in oil, 180 mg, 7.48 mmol, 60%) at 0° C. The mixture was stirred at 0° C. for 1 h, followed by addition of a solution of 28-2 (682 mg, 3.74 mmol) in 1 mL of DMF. The mixture was stirred at room temperature for 2 h. Water was added and the mixture was extracted with ethyl acetate. The organic layer was washed with water, dried over Na₂SO₄, filtered and concentrated. The crude product was purified by silica gel column chromatography (petroleum ether/ethyl acetate=5/1) to afford 28-3.

Step 2: A solution of 28-3 (300 mg, 0.9 mol) in dichloromethane/TFA (6 mL, 2/1) was stirred at room temperature for 1 h. The mixture was concentrated to afford 28-4 which was used in the next step directly without further purification.

Step 3: To a solution of 28-4 (150 mg, 0.64 mmol) and 28-5 (107 mg, 0.77 mmol) in dichloromethane (5 mL) was added DIEA (416 mg, 3.22 mmol, 560.20 uL) and HATU (364 mg, 0.97 mmol). The mixture was stirred at room temperature for 1 h, quenched with H₂O and extracted with dichloromethane. The combined organic layers were washed with brine, dried over anhydrous sodium sulfate, filtered and concentrated. The residue was purified by prep-HPLC (acetonitrile with 0.1% of FA in water: 5% to 95%) to afford 28-6.

Step 4: To a solution of 28-6 (100 mg, 0.28 mmol) and 27-1 (116 mg, 0.34 mmol) in toluene (5 mL) was added RockPhos (13 mg, 0.028 mmol), Pd₂ (dba)₃ (52 mg, 0.057 mmol) and K₃PO₄ (120 mg, 0.057 mmol). The mixture was stirred at 100° C. overnight. The mixture was cooled, diluted with H₂O and extracted with ethyl acetate. The combined organic layers were washed with brine, dried over anhydrous sodium sulfate, filtered and concentrated. The residue was purified by silica gel chromatography (petroleum ether/ethyl acetate=5/1) to afford 28-7.

Step 5: A solution of 28-7 (20 mg, 0.03 mmol) in dichloromethane/TFA (4 mL, 1/1) was stirred at room temperature for 1 h. The mixture was concentrated. The residue was dissolved in THF/H₂O (4 mL, 1/1) and treated with LiOH (3.6 mg, 0.015 mmol). The mixture was stirred at room temperature for 1 h. Then the mixture was purified by prep-HPLC (acetonitrile with 0.1% of FA in water: 5% to 95%) to afford 28. LC-MS: (ESI, m/z): [M+H]+=530.2. ¹H NMR (400 MHZ, methanol-d₄): δ 8.91 (d, J=23.2 Hz, 2H), 7.56-7.37 (m, 3H), 7.32 (t, J=8.1 Hz, 1H), 6.00-5.55 (m, 1H), 4.09-3.56 (m, 4H), 2.64-2.46 (m, 3H), 2.43-2.25 (m, 2H).

### Example 13 Synthesis of Compounds 31 and 32

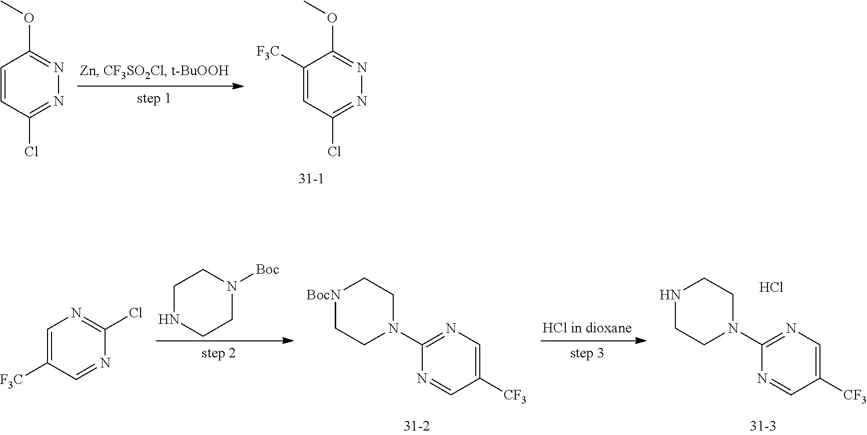

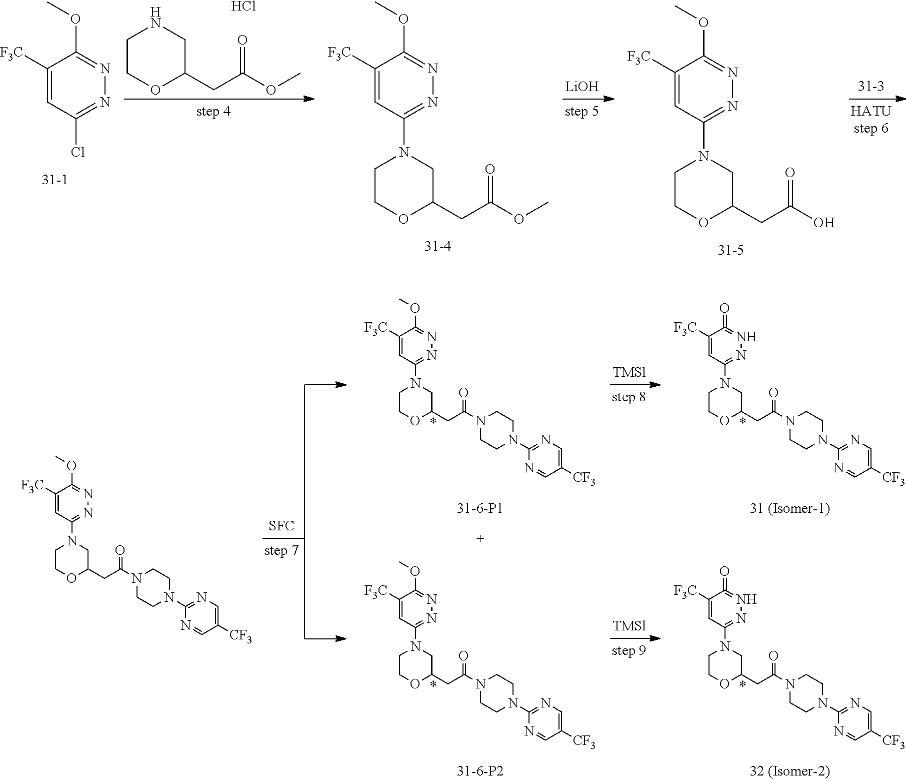

Step 1: To a suspension of Zn (600 mesh, 117 g, 1.8 mol) in H₂O (200 mL) was added CF₃SO₂Cl (50.5 g, 0.30 mol) dropwise with vigorous stirring at 5-10° C. under N₂. The reaction mixture was allowed to warm to room temperature and stirred for another 2 h before it was filtered and the filter cake was washed with H₂O (150 mL). The combined filtrates were added to a solution of 3-chloro-6-methoxypyridazine (10.8 g, 75.0 mmol) in perfluorohexane (150 mL) dropwise at 5-10° C. under N₂. Then tert-butyl hydroperoxide (70% solution in water, 48.5 g, 376.8 mmol) was added dropwise at 5-10° C. under N₂. The reaction mixture was allowed to warm to room temperature and stirred overnight. The mixture was extracted with methyl tert-butyl ether. The combined organic layers were dried over sodium sulfate, filtered and concentrated. The residue was purified by column chromatography on silica gel (petroleum ether/ethyl acetate=50/1) to afford 31-1.

Step 2: To a solution of 2-chloro-5-(trifluoromethyl) pyrimidine (20.0 g, 109.6 mmol) in NMP (160 mL) was added tert-butyl piperazine-1-carboxylate (20.4 g, 109.6 mmol) and K₂CO₃ (30.3 g, 219.2 mmol). The mixture was stirred at 80° C. for 15 h. The mixture was poured into H₂O. The precipitate formed was collected by filtration, washed with water, dried to afford 31-2 which was used in the next step without purification.

Step 3: To a solution of 31-2 (28.4 g, 88.5 mmol) in dichloromethane (284 mL) was added HCl in dioxane (119.8 mL, 4 M in dioxane, 479.0 mmol) dropwise at room temperature. The mixture was stirred at room temperature for 15 h. The precipitate formed was collected by filtration, washed with dichloromethane and dried in vacuo to afford 31-3.

Step 4: To a solution of 31-1 (120 mg, 0.56 mmol) in ethanol (2 mL) was added methyl 2-(morpholin-2-yl) acetate hydrochloride (221 mg, 1.12 mmol) and potassium carbonate (310 mg, 2.24 mmol). The reaction mixture was stirred at 100° C. for 16 h in a sealed tube. The mixture was cooled, extracted with ethyl acetate and washed with water. The combined organic layers were dried over sodium sulfate, filtered and concentrated to afford 31-4 which was used directly in the next step without purification.

Step 5: To a solution of 31-4 (400 mg, 1.2 mmol) in methanol/water (6 mL/6 mL) was added lithium hydroxide monohydrate (150 mg, 3.6 mmol). The reaction was stirred at room temperature for 1 h. The mixture was concentrated and purified by prep-HPLC (acetonitrile with 0.05% of TFA in water: 5% to 50%) to afford 31-5.

Step 6: To a solution of 31-5 (30 mg, 0.09 mmol) in N,N-dimethylformamide (5 mL) were added 31-3 (25 mg, 0.09 mmol), N,N-diisopropylethylamine (35 mg, 0.27 mmol) and HATU (34 mg, 0.09 mmol). The reaction mixture was stirred at room temperature for 1 h. The mixture was extracted with ethyl acetate and washed with water. The combined organic layers were dried over sodium sulfate, filtered and concentrated. The residue was purified by column chromatography on silica gel (petroleum ether/ethyl acetate=1/2) to afford 31-6.

Step 7: Compound 31-6 (40 mg) was purified by SFC (column: DAICEL CHIRALPAK®IC, MeOH (+0.1% 7.0 M ammonia in MeOH)/CO₂) to afford 31-6-P1 (10 mg) and 31-6-P2 (13 mg), respectively. 31-6-P1: SFC analysis: >99% ee; Retention time: 1.00 min; column: DAICEL CHIRALPAK®IC, EtOH (0.1% of DEA) in CO₂; pressure: 100 bar; flow rate: 1.0 mL/min. 31-6-P2: SFC analysis: >99% ee; Retention time: 1.53 min; column: DAICEL CHIRALPAK®IC, EtOH (0.1% of DEA) in CO₂; pressure: 100 bar; flow rate: 1.0 mL/min.

Step 8: To a solution of 31-6-P1 (10 mg, 0.019 mmol) in acetonitrile (2 mL) was added iodotrimethylsilane (8 mg, 0.038 mmol). The mixture was stirred at 70° C. for 3 h. The mixture was purified by prep-HPLC (acetonitrile with 0.05% of TFA in water: 5% to 50%) to afford 31 as 0.9 eq. of TFA salt. LCMS (ESI, m z): [M+H]⁺=522.3; ¹H NMR (400 MHZ, DMSO-d₆) δ 12.75 (s, 1H), 8.72 (s, 2H), 7.92 (s, 1H), 3.88-3.79 (m, 7H), 3.68-3.64 (m, 1H), 3.58-3.53 (m, 5H), 2.79-2.66 (m, 2H), 2.56-2.51 (m, 2H). ¹⁹F NMR (376 MHZ, DMSO-d₆)8-59.32 (3F), -65.77 (3F).

Step 9: To a solution of 31-6-P2 (11 mg, 0.02 mmol) in acetonitrile (2 mL) was added iodotrimethylsilane (8 mg, 0.04 mmol). The mixture was stirred at 70° C. for 3 h. The mixture was purified by prep-HPLC (acetonitrile with 0.05% of TFA in water: 5% to 50%) to afford 32 as 1.8 eq. of TFA salt. LCMS (ESI, m z): [M+H]⁺=522.3; ¹H NMR (400 MHZ, DMSO-d₆): δ 12.76 (s, 1H), 8.72 (s, 2H), 7.92 (s, 1H), 3.88-3.79 (m, 7H), 3.68-3.64 (m, 1H), 3.58-3.53 (m, 5H), 2.79-2.66 (m, 2H), 2.56-2.51 (m, 2H). ¹⁹F NMR (376 MHz, DMSO-d₆): δ -59.32 (3F), -65.77 (3F).

### Example 14 Synthesis of Compounds 36

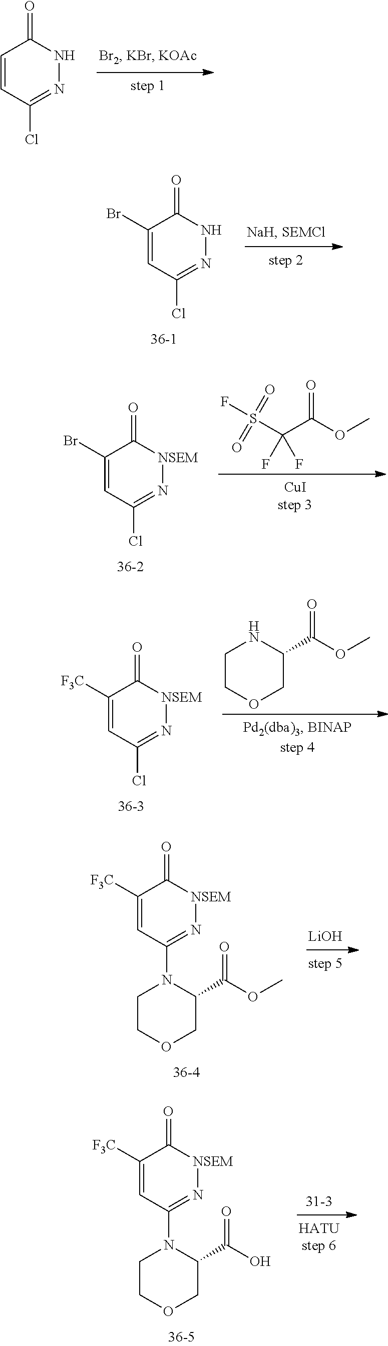

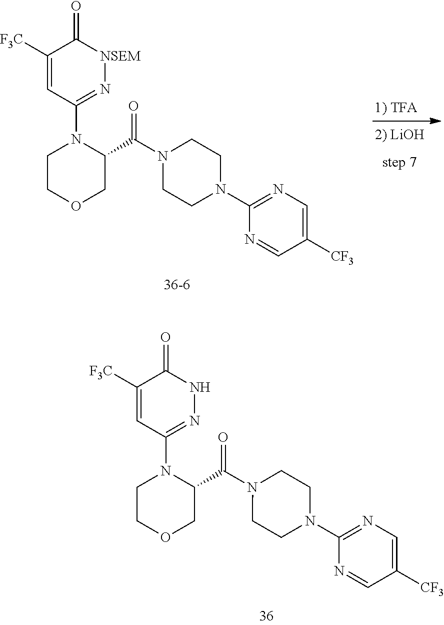

Step 1: To a solution of 6-chloropyridazin-3 (2H)-one (4.0 g, 30.5 mmol) in water (50 mL) was added potassium bromide (10.9 g, 91.6 mmol), potassium acetate (4.5 g, 45.8 mmol) and bromine (14.3 g, 91.6 mmol). The mixture was stirred at 100° C. for 2 h. The mixture was cooled to room temperature and filtered. The filter cake was washed with a solution of sodium sulfite (7.66 g, 60.7 mmol) in water (400 mL) and water (300 mL). The filter cake was dried to afford 36-1 which was used directly in the next step without purification.

Step 2: To a solution of 36-1 (3.4 g, 16.3 mmol) in dimethylformamide (80 mL) was added sodium hydride (1.3 g, 32.6 mmol, 60% in oil) at 0° C. The mixture was stirred at 0° C. for 30 min. 2-(trimethylsilyl) ethoxymethyl chloride (5.4 g, 32.6 mmol) was added. The reaction was stirred at room temperature for 2 h. The mixture was quenched with saturated ammonium chloride aqueous solution and extracted with ethyl acetate. The combined organic layers were dried over sodium sulfate, filtered and concentrated. The residue was purified by column chromatography on silica gel (petroleum ether/ethyl acetate=10/1) to afford 36-2.

Step 3: To a solution of 36-2 (3.2 g, 9.4 mmol) in dimethylformamide (100 mL) was added methyl 2,2-difluoro-2-(fluorosulfonyl) acetate (5.4 g, 28.2 mmol) and copper (I) iodate (1.8 g, 9.4 mmol). The reaction was stirred at 100° C. for 2 h under nitrogen. The mixture was cooled, filtered, extracted with ethyl acetate and washed with water. The combined organic layers were dried over sodium sulfate, filtered and concentrated. The residue was purified by column chromatography on silica gel (petroleum ether/ethyl acetate=10/1) to afford 36-3.

Step 4: To a solution of 36-3 (180 mg, 0.55 mmol) in dioxane (2 mL) was added methyl(S)-morpholine-3-carboxylate (120 mg, 0.82 mmol), cesium carbonate (360 mg, 1. 1 mmol), tris (dibenzylideneacetone) dipalladium (0) (55 mg, 0.06 mmol) and (+)-2,2′-bis (diphenylphosphino)-1, l′-binaphthalene (62 mg, 0.1 mmol). The reaction was stirred at 120° C. for 16 h in a sealed tube. The mixture was cooled, concentrated and purified by column chromatography on silica gel (petroleum ether/ethyl acetate=2/1) to afford 36-4.

Step 5: To a solution of 36-4 (140 mg, 0.34 mmol) in methanol/water (5 mL/5 mL) was added lithium hydroxide monohydrate (42 mg, 1 mmol). The mixture was stirred at room temperature for 1 h. The mixture was quenched with 1 M HCl to adjust the pH to 5 and extracted with dichloromethane. The combined organic layers were concentrated to afford 36-5 which was used directly in the next step without purification.

Step 6: To a solution of 36-5 (120 mg, 0.28 mmol) in N,N-dimethylformamide (6 mL) was added 31-3 (76 mg, 0.28 mmol), N,N-diisopropylethylamine (110 mg, 0.85 mmol) and HATU (108 mg, 0.28 mmol). The reaction mixture was stirred at room temperature for 1 h. The mixture was extracted with ethyl acetate and washed with water. The combined organic layers were dried over sodium sulfate, filtered and concentrated. The residue was purified by column chromatography on silica gel (petroleum ether/ethyl acetate=1/2) to afford 36-6.

Step 7: To a solution of 36-6 (60 mg, 0.12 mmol) in dichloromethane (4 mL) was added trifluoroacetic acid (1 mL). The reaction was stirred at room temperature for 1 h. The mixture was washed with saturated sodium bicarbonate aqueous solution and extracted with dichloromethane. The combined organic layers were concentrated. The residue was dissolved in methanol/water (1 mL/0.5 mL) and lithium hydroxide monohydrate (15 mg, 0.36 mmol) was added. The reaction was stirred at room temperature for 1 h. The mixture was purified by prep-HPLC (acetonitrile with 0.05% of TFA in water: 5% to 50%) to afford 36 as a 0.4 eq of TFA salt. LCMS (ESI, m z): [M+Na]+=530.2; ¹H NMR (400 MHZ, DMSO-d₆): δ 12.81 (s, 1H), 8.74 (s, 2H), 7.92 (s, 1H), 4.83 (s, 1H), 4.15-4.11 (m, 2H), 3.97-3.81 (m, 3H), 3.79-3.69 (m, 4H), 3.62-3.58 (m, 3H), 3.42-3.38 (m, 1H), 3.23-3.13 (m, 1H). ¹⁹F NMR (376 MHZ, DMSO-d₆)8-59.31 (3F), -65.81 (3F).

### Example 15 Synthesis of Compounds 44 and 45

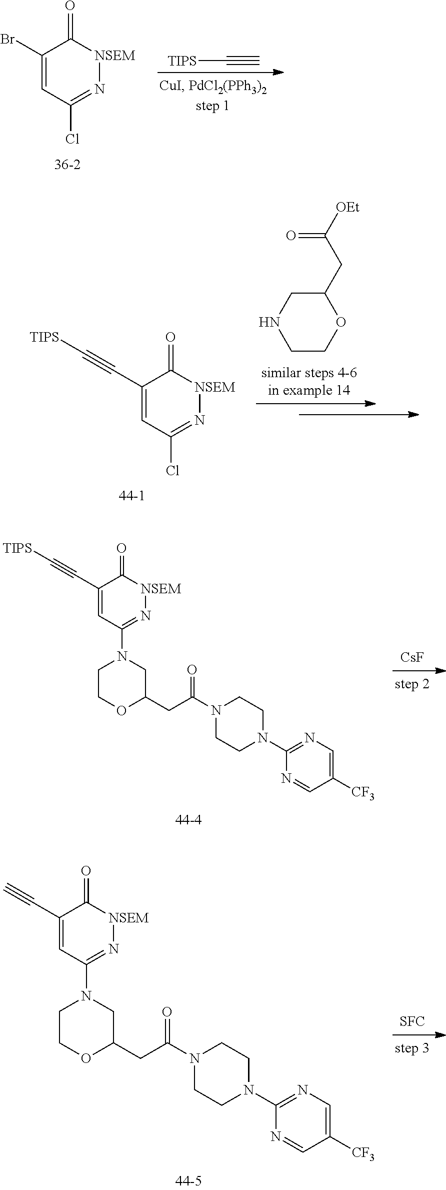

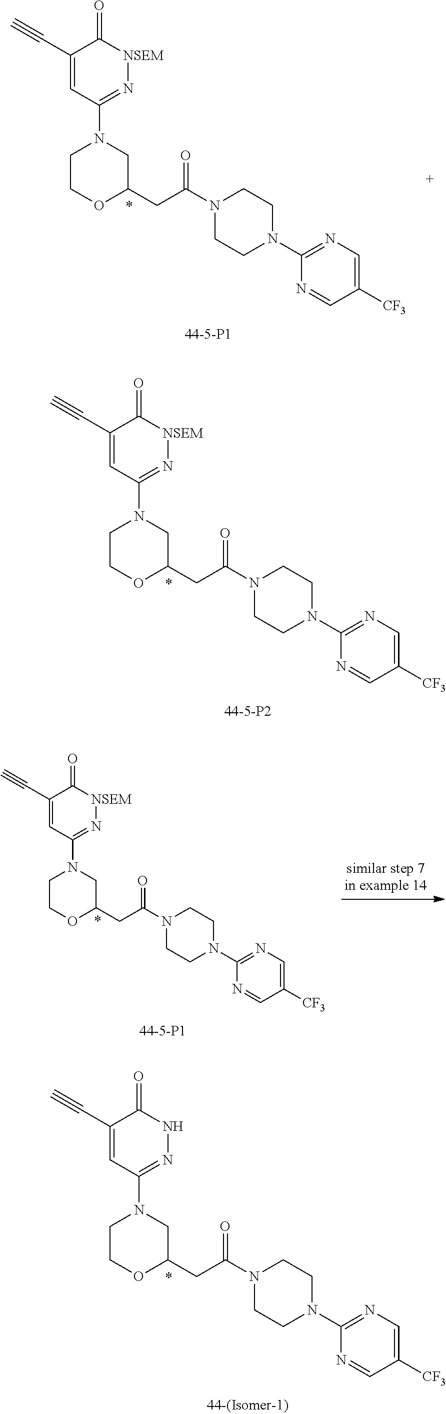 [SMILES: C#Cc1cc(N2CCOC(CC(=O)N3CCN(c4ncc(C(F)(F)F)cn4)CC3)C2)nn(COCC[Si](C)(C)C)c1=O ; C#Cc1cc(N2CCOC(CC(=O)N3CCN(c4ncc(C(F)(F)F)cn4)CC3)C2)nn(COCC[Si](C)(C)C)c1=O ; C#Cc1cc(N2CCOC(CC(=O)N3CCN(c4ncc(C(F)(F)F)cn4)CC3)C2)nn(COCC[Si](C)(C)C)c1=O ; C#Cc1cc(N2CCOC(CC(=O)N3CCN(c4ncc(C(F)(F)F)cn4)CC3)C2)n[nH]c1=O]

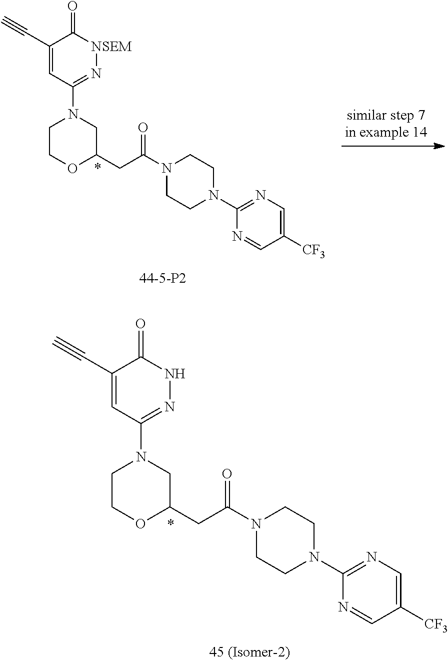 [SMILES: C#Cc1cc(N2CCOC(CC(=O)N3CCN(c4ncc(C(F)(F)F)cn4)CC3)C2)nn(COCC[Si](C)(C)C)c1=O ; C#Cc1cc(N2CCOC(CC(=O)N3CCN(c4ncc(C(F)(F)F)cn4)CC3)C2)n[nH]c1=O]

Step 1: A mixture of 36-2, ethynyltriisopropylsilane (2.42 g, 13.25 mmol), Cul (841 mg, 4.42 mmol) and Pd(PPh₃)₂Cl₂ (1.24 g, 1.77 mmol) in DMF (5 mL) and TEA (50 mL) was stirred at room temperature for 16 h. The mixture was poured into water. The resulting solution was extracted with ethyl acetate. The combined organic layers were dried over anhydrous sodium sulfate, filtered and concentrated. The residue was purified by column chromatography on silica gel (petroleum ether/ethyl acetate=3/1) to afford 44-1.

Compound 44-4 was prepared from compound 44-1 following the procedure for the synthesis of compound 36-6 in example 14.

Step 2: A mixture of 44-4 (160 mg, 0.2 mmol) and CsF (138 mg, 2.1 mmol) in DMF (10 mL) was stirred at room temperatures for 2 h. The mixture was purified by reverse phase HPLC (acetonitrile with 0.1% of FA in water: 5% to 95%) to afford 44-5.

Step 3:44-5 (150 mg) was purified by SFC (column: REGIS (S,S) WHELK-01, MeOH (+0.1% 7.0 mol/L ammonia in MeOH)/CO₂=50/50) to afford 44-5-P1 (45 mg) and 44-5-P2 (65 mg), respectively. 44-5-P1: SFC analysis: 98.12% ee; retention time: 2.32 min; column: REGIS (S,S) WHELK-01, MeOH (0.1% of DEA) in CO₂, 40%; pressure: 100 bar; flow rate: 1.5 mL/min. 44-5-P2: SFC analysis: 97.56% ee; retention time: 2.95 min; column: REGIS (S,S) WHELK-01, MeOH (0.1% of DEA) in CO₂, 40%; pressure: 100 bar; flow rate: 1.5 mL/min.

Compound 44 was prepared from compound 44-5-P1 following the procedure for the synthesis of compound 36 in example 14. LCMS (ESI, m z): [M+H]⁺=478.2; ¹H NMR (400 MHZ, methanol-d₄): δ 8.59 (s, 2H), 7.71 (s, 1H), 4.10 (s, 1H), 4.06-3.61 (m, 13H), 2.90-2.79 (m, 2H), 2.67-2.53 (m, 2H).

Compound 45 was prepared from compound 44-5-P2 following the procedure for the synthesis of compound 36 in example 14. LCMS (ESI, m z): [M+H]⁺=478.2; ¹H NMR (400 MHZ, methanol-d₄): δ 8.59 (s, 2H), 7.71 (s, 1H), 4.12 (s, 1H), 4.03-3.63 (m, 13H), 2.89-2.79 (m, 2H), 2.68-2.53 (m, 2H).

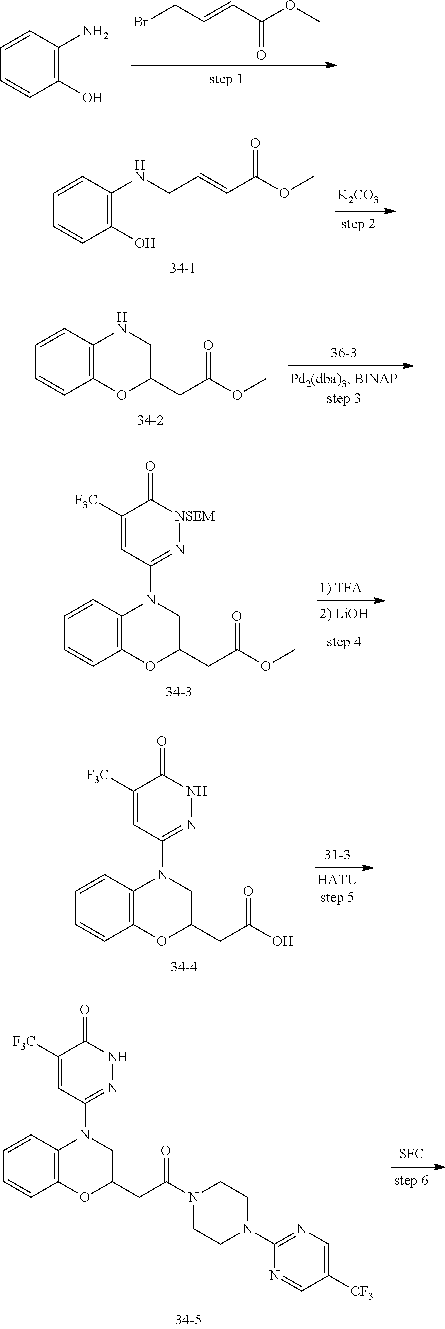

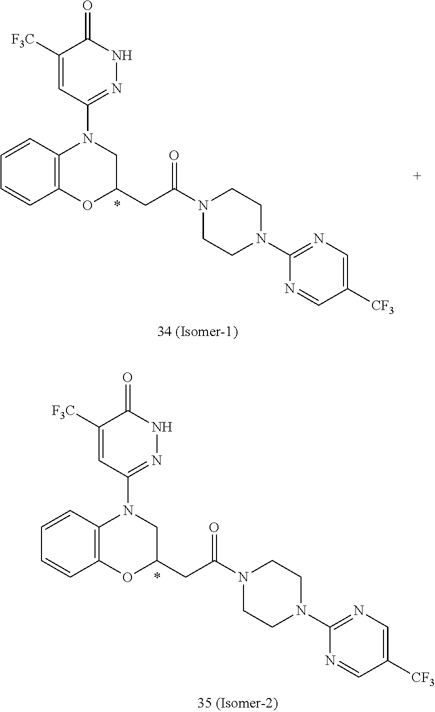 [SMILES: O=C(CC1CN(c2cc(C(F)(F)F)c(=O)[nH]n2)c2ccccc2O1)N1CCN(c2ncc(C(F)(F)F)cn2)CC1 ; O=C(CC1CN(c2cc(C(F)(F)F)c(=O)[nH]n2)c2ccccc2O1)N1CCN(c2ncc(C(F)(F)F)cn2)CC1]

Step 1: To a solution of 2-aminophenol (1.1 g, 10.1 mmol) in MeOH (10 mL) was added NaHCO₃ (1.0 g, 12.1 mmol). Then methyl (E)-4-bromobut-2-enoate (1.8 g, 10.1 mmol) was added dropwise. The mixture was stirred at room temperature for 3 h. The mixture was filtered and concentrated. The crude product was purified by column chromatography on silica gel (petroleum ether/ethyl acetate=3/1) to afford 34-1.

Step 2: To a solution of 34-1 (1.5 g, 7.24 mmol) in MeOH (5 mL) was added K₂CO₃ (100 mg, 0.72 mmol). The mixture was stirred at room temperature for 1 h. The mixture was extracted with ethyl acetate and washed with water. The combined organic layers were dried over sodium sulfate, filtered and concentrated. The residue was purified by reverse phase HPLC (acetonitrile with 0.1% of FA in water: 5% to 50%) to afford 34-2.

Step 3: To a solution of 34-2 (473 mg, 2.28 mmol) and 36-3 (500 mg, 1.52 mmol) in dioxane (10 mL) was added Pd₂ (dba)₃ (139 mg, 0.15 mmol), BINAP (190 mg, 0.30 mmol), Cs₂CO₃ (546 mg, 1.67 mmol). Then the mixture was stirred at 120° C. under N₂ overnight. The mixture was cooled, diluted with water and extracted with ethyl acetate. The combined organic layers were dried over sodium sulfate, filtered and concentrated. The residue was purified by reverse phase HPLC (acetonitrile with 0.1% of FA in water: 5% to 95%) to afford 34-3.

Step 4: A solution of 34-3 (200 mg, 0.4 mmol) in dichloromethane/TFA (3 mL/1 mL). The mixture was stirred at room temperature for 1 h. The mixture was concentrated. The residue was dissolved in MeOH/H₂O (2 mL/2 mL). LiOH (48 mg, 2.00 mmol) was added. The mixture was stirred at room temperature for 1 hour. The mixture was purified by reverse phase HPLC (acetonitrile with 0.1% of FA in water: 5% to 95%) to afford 34-4.

Step 5: To a solution of 34-4 (100 mg, 0.28 mmol) and 31-3 (65 mg, 0.28 mmol) in DMF (2 mL) were added DIEA (182 mg, 1.41 mmol) and HATU (159 mg, 0.42 mmol). Then the mixture was stirred at room temperature for 1 h. The residue was purified by prep-HPLC (acetonitrile with 0.1% of FA in water: 5% to 95%) to afford 34-5.

Step 6: Compound 34-5 (60 mg) was purified by SFC (column: DAICEL CHIRALPAK®OJ, MeOH (+0.1% 7.0 mol/L ammonia in MeOH)/CO₂=70/30) to afford 34 (10.2 mg) and 35 (9.5 mg), respectively. 34: SFC analysis: >99% ee; retention time: 3.38 min; column: DAICEL CHIRALPAK®OJ, MeOH (0.1% of DEA) in CO₂, 5% to 40%; pressure: 100 bar; flow rate: 1.5 mL/min. LCMS (ESI, m z): [M+H]=570.3; ¹H NMR (400 MHZ, methanol-d₄): δ 8.59 (s, 2H), 7.98 (s, 1H), 7.01-6.84 (m, 4H), 4.80-4.65 (m, 1H), 4.20-4.14 (m, 1H), 4.01-3.90 (m, 4H), 3.78-3.65 (m, 4H), 3.56-3.49 (m, 1H), 3.06-2.97 (m, 1H), 2.83-2.76 (m, 1H). 35: SFC analysis: 98.58% ee; retention time: 3.81 min; column: DAICEL CHIRALPAK®OJ, MeOH (0.1% of DEA) in CO₂, 5% to 40%; pressure: 100 bar; flow rate: 1.5 mL/min. LCMS (ESI, m z): [M+H]=570.3; ¹H NMR (400 MHZ, methanol-d₄): δ 8.59 (s, 2H), 7.96 (s, 1H), 6.99-6.86 (m, 4H), 4.81-4.66 (m, 1H), 4.19-4.13 (m, 1H), 4.03-3.85 (m, 4H), 3.79-3.62 (m, 4H), 3.57-3.49 (m, 1H), 3.05-2.98 (m, 1H), 2.83-2.76 (m, 1H).

### Example 17 Synthesis of Compounds 42 and 43

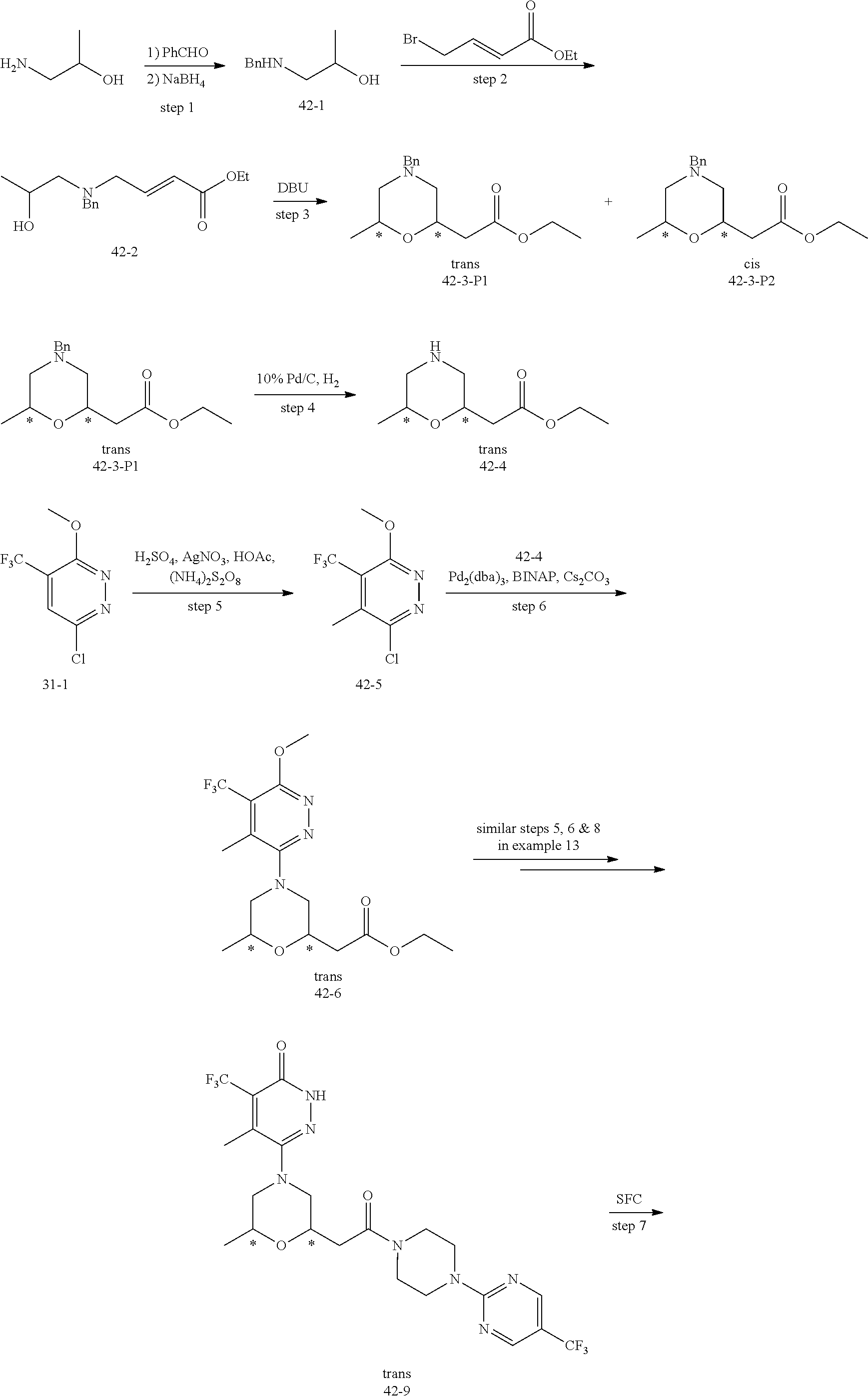

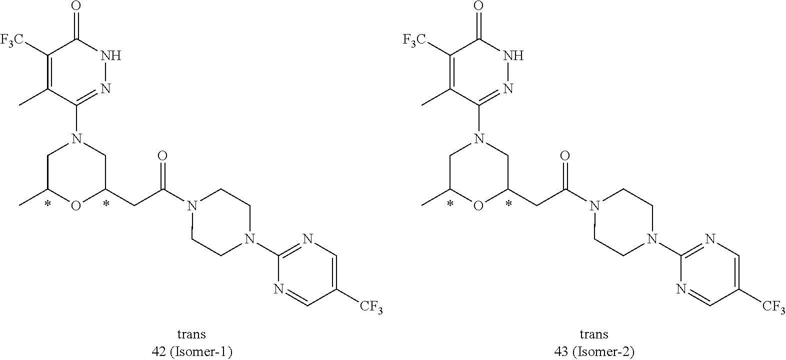 [SMILES: Cc1c(N2CC(C)OC(CC(=O)N3CCN(c4ncc(C(F)(F)F)cn4)CC3)C2)n[nH]c(=O)c1C(F)(F)F ; Cc1c(N2CC(C)OC(CC(=O)N3CCN(c4ncc(C(F)(F)F)cn4)CC3)C2)n[nH]c(=O)c1C(F)(F)F]

Step 1: A mixture of benzaldehyde (7 g, 66 mmol) and 1-aminopropan-2-ol (5 g, 67 mmol) in EtOH (330 mL) was stirred at reflux temperature for 6 h. After being cooled to room temperature, NaBH₄ (3.8 g, 100.45 mmol) was added in portions under ice-water bath and the resulting mixture was stirred at room temperature for 4 h. The reaction was quenched with water. The mixture was extracted with dichloromethane. The combined organic layers were washed with brine, dried over anhydrous Na₂SO₄, filtered and concentrated. The residue was purified by column chromatography on silica gel (petroleum ether/ethyl acetate=3/1) to afford 42-1.

Step 2: To a solution of 42-1 (5.74 g, 34.74 mmol) and TEA (7 g, 69.18 mmol) in dichloromethane (115 mL) was added ethyl (E)-4-bromobut-2-enoate (6.2 g, 34.64 mmol). The mixture was stirred at room temperature for 16 h. Another portion of TEA (3.5 g, 34.65 mmol) and (E)-4-bromobut-2-enoate (3.1 g, 17.32 mmol) was added and the resulting mixture was stirred for 4 h. The mixture was concentrated and the residue was purified by column chromatography on silica gel (petroleum ether/ethyl acetate=2/1) to afford 42-2.

Step 3: To a solution of 42-2 (2.99 g, 11.35 mmol) in toluene (60 mL) was added DBU (1.73 g, 11.36 mmol). The reaction mixture was stirred at 100° C. for 2 h. The mixture was cooled, concentrated and the residue was purified by column chromatography on silica gel (petroleum ether/ethyl acetate=2/1) to afford 42-3-P1 (less polar fraction) and 42-3-P2 (more polar fraction).

Step 4: A mixture of 42-3-P1 (2.8 g, 10.10 mmol) and 10% wet palladium on carbon (1.25 g) in EtOH (150 mL) was stirred at room temperature for 16 h under 1 atm H₂ atmosphere before it was filered and the filtrate was concentrated. The residue was purified by column chromatography on silica gel (dichloromethane/methanol=5/1) to afford 42-4.

Step 5: Under N₂. H₂SO₄ (98%, 5.0 g, 51 mmol) was added to H₂O (75 mL), then 31-1 (5.0 g, 23.5 mmol), AgNO₃ (800 mg, 4.7 mmol) and AcOH (5.0 g, 83.5 mmol) were added successively. The mixture was heated at 55° C. and a solution of (NH₄)₂S₂O₈ (13.5 g, 59.0 mmol) in H₂O (30 mL) was added dropwise over 0.5 h. The resulting mixture was stirred at 55° C. for 1 h. The mixture was cooled and extracted with methyl tert-butyl ether. The combined organic layers were dried over sodium sulfate, filtered and concentrated. The residue was purified by reverse phase HPLC (acetonitrile with 0.05% of TFA in water: 5% to 95%) to afford 42-5.

Step 6: To a solution of 42-5 (380 mg, 1.68 mmol) in dioxane (10 mL) were added 42-4 (673 mg, 2.52 mmol), Pd₂ (dba)₃ (154 mg, 0.17 mmol), BINAP (210 mg, 0.34 mmol) and Cs₂CO₃ (1.1 g, 3.35 mmol). The resulting mixture was stirred at 120° C. for 4 h under N₂. The mixture was cooled, diluted with ethyl acetate and washed with sat. aq. NaHCO₃ solution. The organic layer was dried over sodium sulfate, filtered and concentrated. The residue was purified by reverse phase HPLC (acetonitrile with 0.05% of TFA in water: 5% to 80%) to afford 42-6.

Compound 42-9 was prepared from compound 42-6 following the procedure for the synthesis of compounds 31 and 32 in example 13.

Step 7: Compound 42-9 (65 mg) was purified by SFC (column: DAICEL CHIRALPAK®OJ, MeOH (+0.1% 7.0 mol/L ammonia in MeOH)/CO₂=85/15) to afford 42 (11.9 mg) and 43 (14.7 mg), respectively. 42: SFC analysis: 98.80% ee; retention time: 2.52 min; column: DAICEL CHIRALPAK®OJ, MeOH (0.1% of DEA) in CO₂, 5% to 40%; pressure: 100 bar; flow rate: 1.5 mL/min. LCMS (ESI, m z): [M+H]=550.0; ¹H NMR (400 MHZ, methanol-d₄): δ 8.59 (s, 2H), 4.46-4.42 (m, 1H), 4.20-4.15 (m, 1H), 4.00-3.85 (m, 4H), 3.77-3.63 (m, 4H), 3.13-3.02 (m, 2H), 2.93-2.88 (m, 3H), 2.75-2.68 (m, 1H), 2.49-2.46 (m, 3H), 1.29 (d, J=6.5 Hz, 3H). 43: SFC analysis: 90.92% ee; retention time: 2.60 min; column: DAICEL CHIRALPAK®OJ, MeOH (0.1% of DEA) in CO₂, 5% to 40%; pressure: 100 bar; flow rate: 1.5 mL/min. LCMS (ESI, m z): [M+H]=550.0; ¹H NMR (400 MHZ, methanol-d₄): δ 8.59 (s, 2H), 4.48-4.40 (m, 1H), 4.20-4.15 (m, 1H), 4.00-3.85 (m, 4H), 3.78-3.62 (m, 4H), 3.14-3.02 (m, 2H), 2.93-2.88 (m, 3H), 2.75-2.68 (m, 1H), 2.49-2.46 (m, 3H), 1.27 (d, J=6.5 Hz, 3H).

### Example 18 Synthesis of Compound 48

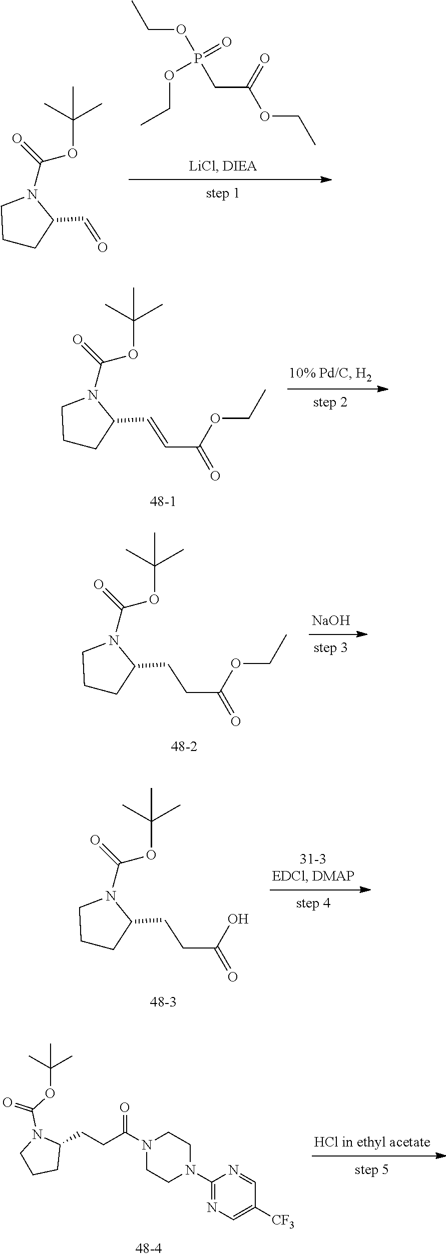

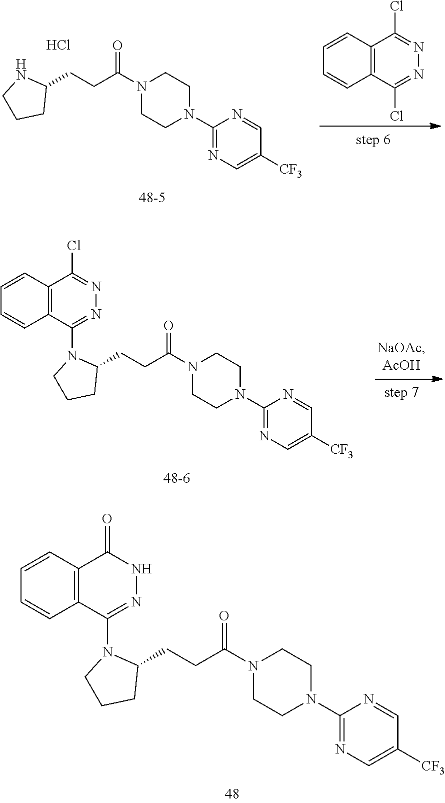

Step 1: tert-butyl(S)-2-formylpyrrolidine-1-carboxylate (2.0 g, 10.0 mmol) was dissolved in acetonitrile (20 mL), followed by addition of LiCI (510 mg, 12.0 mmol) and DIPEA (1.6 g, 12.0 mmol) and triethyl phosphonoacetate (2.7 g, 12.0 mmol) successively at room temperature. The mixture was stirred at room temperature for 4 h. The mixture was diluted with water and extracted with ethyl acetate. The combined organic layers were washed with water, dried over MgSO₄, filtered, and concentrated. The residue was purified by column chromatography on silica gel (petroleum ether/ethyl acetate=10/1 to 5/1) to afford 48-1.

Step 2: To a solution of 48-1 (1.0 g, 3.7 mmol) in MeOH (10.0 mL) was added 10% Pd/C (200 mg). The mixture was stirred at room temperature for 3 h under H₂. The mixture was filtered and the filtrate was concentrated to afford 48-2 which was used directly in the next step without purification.

Step 3: To a solution of 48-2 (800 mg, 2.9 mmol) in methanol/water (5 mL/2 mL) was added NaOH (580 mg, 14.5 mmol). The mixture was stirred at room temperature for 5 h. The organic solvent was removed. The resulting aqueous solution was acidified to pH=3 with 1N HCl and then extracted with dichloromethane. The combined organic layers were dried over sodium sulfate, filtered and concentrated to afford 48-3 which was used directly in the next step without purification.

Step 4: To a mixture of 48-3 (400 mg, 1.6 mmol), 31-3 (552 mg, 1.8 mmol), DIEA (620 mg, 4.8 mmol) and DMAP (39 mg, 0.3 mmol) in dichloromethane (10 mL) was added EDCI (368 mg, 1.9 mmol) at room temperature. Then the mixture was stirred at room temperature overnight. The mixture was diluted with water and extracted with ethyl acetate. The combined organic layers were washed with water, dried over sodium sulfate, filtered, and concentrated. The residue was purified by column chromatography on silica gel (petroleum ether/ethyl acetate=20/1) to afford 48-4.

Step 5: A solution of 48-4 (750 mg, 1.6 mmol) in a solution of HCl in ethyl acetate (5.5 mL, 2 M) was stirred at room temperature for 2 h. The mixture was concentrated. The crude was triturated with methyl tert-butyl ether and filtered to afford 48-5.

Step 6: To a solution of 48-5 (400 mg, 1.0 mmol) and 1,4-dichlorophthalazine (438 mg, 2.2 mmol) in NMP (5 mL) was added Cs₂CO₃ (2.2 g, 6.6 mmol) at room temperature. The reaction mixture was stirred at 80° C. for 48 h. The mixture was cooled, diluted with water and extracted with ethyl acetate. The combined organic layers were washed with water, dried over sodium sulfate, filtered, and concentrated. The residue was purified by column chromatography on silica gel (petroleum ether/ethyl acetate=2/1) to afford 48-6.

Step 7: To a solution of 48-6 (100 mg, 0.2 mmol) and NaOAc (162 mg, 2.0 mmol) in DMAc (3 mL) was added 5 drops AcOH at room temperature. The mixture was cooled, diluted with water and extracted with ethyl acetate. The combined organic layers were washed with water, dried over sodium sulfate, filtered, and concentrated. The residue was purified by prep-HPLC (acetonitrile with 0.1% of FA in water: 5% to 95%) to afford 48. LCMS (ESI, m z): [M+H]⁺=501.9; ¹H NMR (400 MHZ, methanol-d₄): δ 8.57 (d, J=0.4 Hz, 2H), 8.34 (dd, J=8.0, 1.2 Hz, 1H), 8.13 (d, J=8.0 Hz, 1H), 7.93-7.91 (m, 1H), 7.85-7.83 (m, 1H), 4.42-4.38 (m, 1H), 3.93-3.90 (m, 1H), 3.81-3.79 (m, 4H), 3.57-3.49 (m, 4H), 3.28-3.27 (m, 1H), 2.50-2.47 (m, 2H), 2.24-2.22 (m, 1H), 2.02-1.99 (m, 2H), 1.86-1.84 (m, 3H). ¹⁹F NMR (376 MHz, methanol-d₄)8-62.66 (3F).

### Example 19 Synthesis of Compounds 49 and 50

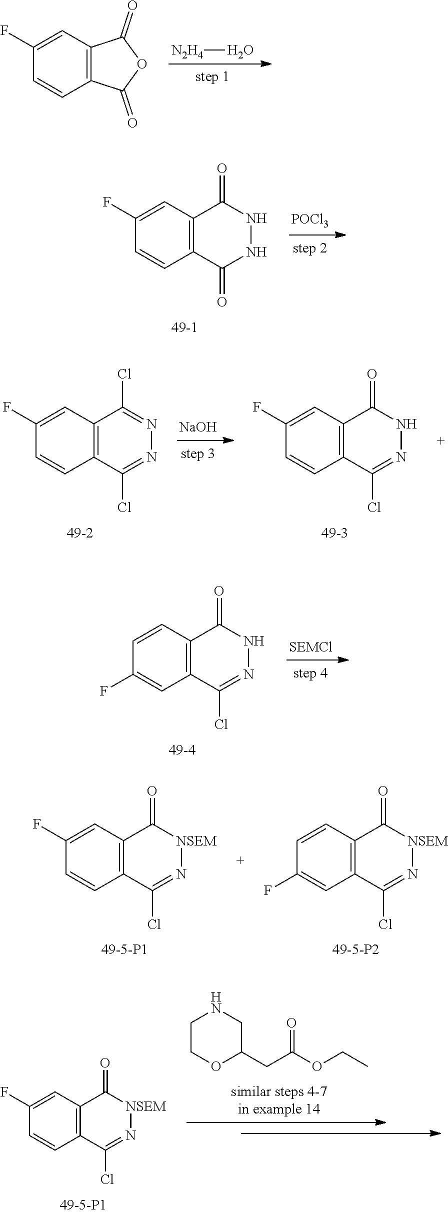

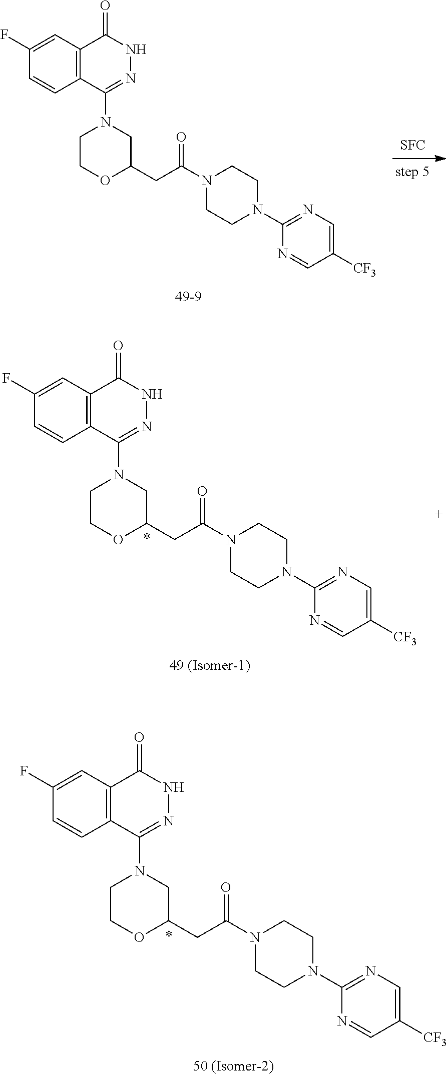 [SMILES: O=C(CC1CN(c2n[nH]c(=O)c3cc(F)ccc23)CCO1)N1CCN(c2ncc(C(F)(F)F)cn2)CC1 ; "SFC" ; O=C(CC1CN(c2n[nH]c(=O)c3cc(F)ccc23)CCO1)N1CCN(c2ncc(C(F)(F)F)cn2)CC1 ; O=C(CC1CN(c2n[nH]c(=O)c3cc(F)ccc23)CCO1)N1CCN(c2ncc(C(F)(F)F)cn2)CC1]

Step 1: To a solution of 5-fluoroisobenzofuran-1,3-dione (1.8 g, 10.84 mmol) in 10% HCl (50 mL) was added N₂H₄—H₂O (1.04 g, 20.8 mmol). The mixture was stirred at 100° C. for 24 h. The mixture was cooled and filtered. The filter cake was washed with water and dried to afford 49-1 which was used directly in the next step without purification.

Step 2: A mixture of 49-1 (1.8 g, 9.99 mmol) in POCI₃ (30 mL) was stirred at 110° C. for 3 h. The mixture was cooled and poured into ice water, then filtered and the filter cake was dried to afford 49-2 which was used directly in the next step without purification.

Step 3: A solution of 49-2 (1.7 g, 7.83 mmol) in 5M NaOH (30 mL) was stirred at room temperature overnight. The mixture was filtered. The filter cake was washed with water and dried to afford a mixture of 49-3 and 49-4 which was used directly in the next step without purification.

Step 4: To a mixture of 49-3 and 49-4 (1.2 g, 6.04 mmol) in DMF (20 mL) was added NaH (60% in oil. 290 mg, 12.09 mmol) at 0° C. The mixture was stirred at 0° C. for 1 h. SEMCI (1.21 g, 7.25 mmol) was added at 0° C. Then the mixture was allowed to warm to room temperature and stirred for 1 h. The mixture was poured into ice water and extracted with ethyl acetate. The combined organic layers were washed with water, dried over sodium sulfate, filtered, and concentrated. The residue was purified by column chromatography on silica gel (petroleum ether/ethyl acetate=10/1) to afford 49-5-P1 (less polar) and 49-5-P2 (more polar) respectively.

Compound 49-9 was prepared from compound 49-5-P1 following the procedure for the synthesis of compound 36 in example 14.

Compound 49-9 (120 mg) was purified by SFC (column: DAICEL CHIRALPAK®OJ, MeOH (+0.1% 7.0 mol/L ammonia in MeOH)/CO₂=75/25) to afford 49 (35 mg) and 50 (29 mg), respectively. 49: SFC analysis: 98.68% ee; retention time: 3.21 min; column: DAICEL CHIRALCEL®OJ, EtOH (0.1% of DEA) in CO₂, 5% to 40%; pressure: 100 bar; flow rate: 1.5 mL/min. LCMS (ESI, m z): [M+H]⁺=522.2; ¹H NMR (400 MHZ, methanol-d₄): δ 8.59 (s, 2H), 8.16 (dd, J=8.9, 5.1 Hz, 1H), 7.97 (dd, J=8.7, 2.7 Hz, 1H), 7.70 (td, J=8.7, 2.7 Hz, 1H), 4.30-4.22 (m, 1H), 4.10-3.83 (m, 6H), 3.82-3.59 (m, 4H), 3.56-3.48 (m, 1H), 3.36-3.32 (m, 1H), 3.06-2.96 (m, 1H), 2.88-2.74 (m, 2H), 2.63-2.55 (m, 1H). 50: SFC analysis: 94.16% ee; retention time: 3.33 min; column: DAICEL CHIRALPAK®OJ, EtOH (0.1% of DEA) in CO₂, 5% to 40%; pressure: 100 bar; flow rate: 1.5 mL/min. LCMS (ESI, m z): [M+H]⁺=522.2; ¹H NMR (400 MHZ, methanol-d₄): δ 8.59 (s, 2H), 8.16 (dd, J=9.0, 5.1 Hz, 1H), 7.97 (dd, J=8.7, 2.7 Hz, 1H), 7.70 (td, J=8.7, 2.8 Hz, 1H), 4.33-4.18 (m, 1H), 4.11-3.84 (m, 6H), 3.81-3.58 (m, 4H), 3.55-3.50 (m, 1H), 3.35-3.32 (m, 1H), 3.06-2.95 (m, 1H), 2.88-2.74 (m, 2H), 2.62-2.54 (m, 1H).

### Example 20 Synthesis of Compound 73

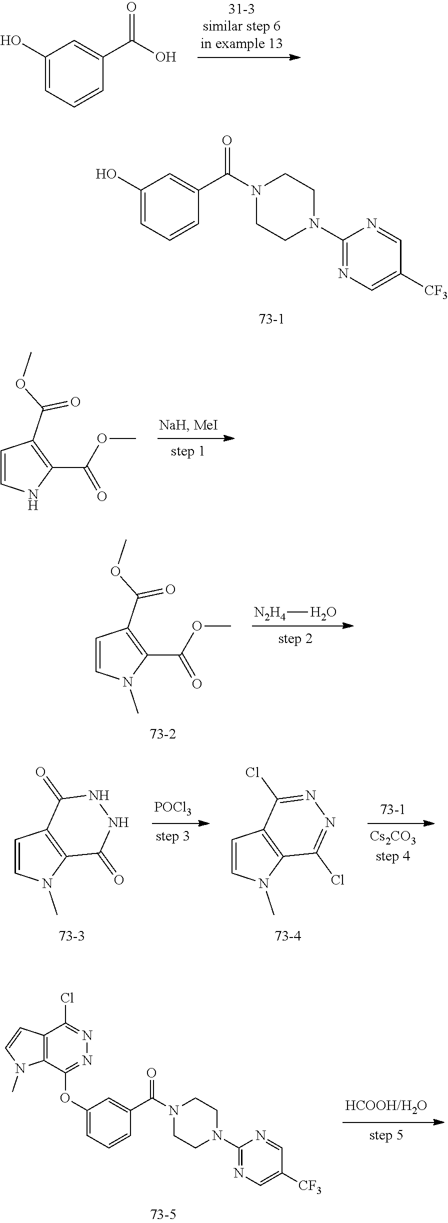

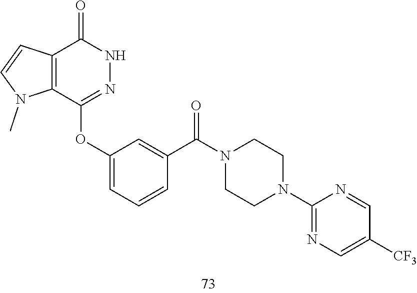 [SMILES: Cn1ccc2c(=O)[nH]nc(Oc3cccc(C(=O)N4CCN(c5ncc(C(F)(F)F)cn5)CC4)c3)c21]

Compound 73-1 was prepared from compound 3-hydroxybenzoic acid following the procedure for the synthesis of compound 31-6 in example 13.

Step 1: To a solution of dimethyl 1H-pyrrole-2,3-dicarboxylate (1.0 g, 5.5 mmol) in DMF (10 mL) was added NaH (440 mg, 11.0 mmol, 60%) in portions at 0° C. The mixture was stirred at 0° C. for 0.5 h, followed by addition of Mel (937 mg, 6.6 mmol). The mixture was stirred at 25° C. for 3 h. The reaction was quenched by sat. aq. NH₄Cl solution and the mixture was extracted with ethyl acetate. The combined organic layers were washed with water, dried over sodium sulfate, filtered, and concentrated to afford 73-2 which was used directly in the next step without purification.

Step 2: To a solution of 73-2 (700 mg, 3.6 mmol) in EtOH (20 mL) was added hydrazine hydrate (80% in water, 676 mg, 13.5 mmol) at room temperature. The mixture was stirred at reflux for 12 h. The mixture was concentrated. The residue was triturated in dichloromethane/methanol (10/1) and filtered to afford 73-3.

Step 3: A solution of 73-3 (150 mg, 0.91 mmol) in POCI₃ (3 mL) was stirred at 80° C. for 3 h. The mixture was concentrated. The residue was diluted with ethyl acetate and washed with sat. aq. NaHCO₃ and brine. The organic layer was dried over Na₂SO₄, filtered and concentrated to afford 73-4 which was used directly in the next step without purification.

Step 4: To a solution of 73-4 (130 mg, 0.64 mmol) in MeCN(2 mL) was added 73-1 (271 mg, 0.77 mmol) and Cs₂CO₃ (626 mg, 1.92 mmol) at room temperature. The mixture was stirred at 60° C. for 12 h. The mixture was cooled, poured into water and the mixture was extracted with ethyl acetate. The combined organic layers were washed with brine, dried over Na₂SO₄, filtered and concentrated. The residue was purified by prep-TLC (petroleum ether/ethyl acetate=10/1) to afford 73-5.

Step 5: A solution of 73-5 (100 mg, 0.19 mmol) in HCOOH (1 mL) and H₂O (1 mL) was stirred at 85° C. for 12 h. The mixture was cooled and adjusted to pH 5 to 6 with 23% NaOH aqueous solution. Then the mixture was extracted with ethyl acetate. The combined organic layers were washed with brine, dried over Na₂SO₄ and concentrated. The residue was purified by prep-HPLC (acetonitrile with 0.1% of FA in water: 30% to 53% to 100%) to afford 73. LCMS (ESI, m z): [M+H]⁺=500.2; ¹H NMR (400 MHZ, methanol-d₄, ppm): δ 8.60 (s, 2H), 7.59-7.54 (m, 1H), 7.42-7.40 (m, 2H), 7.37-7.35 (m, 2H), 6.82 (d, J=3.2 Hz, 1H), 4.10 (s, 3H), 4.04-3.96 (m, 4H), 3.86-3.83 (m, 2H), 3.61-3.58 (m, 2H). ¹⁹F NMR (376 MHz, methanol-d₄, ppm): δ-62.66 (3F).

### Example 21 Synthesis of compound 75

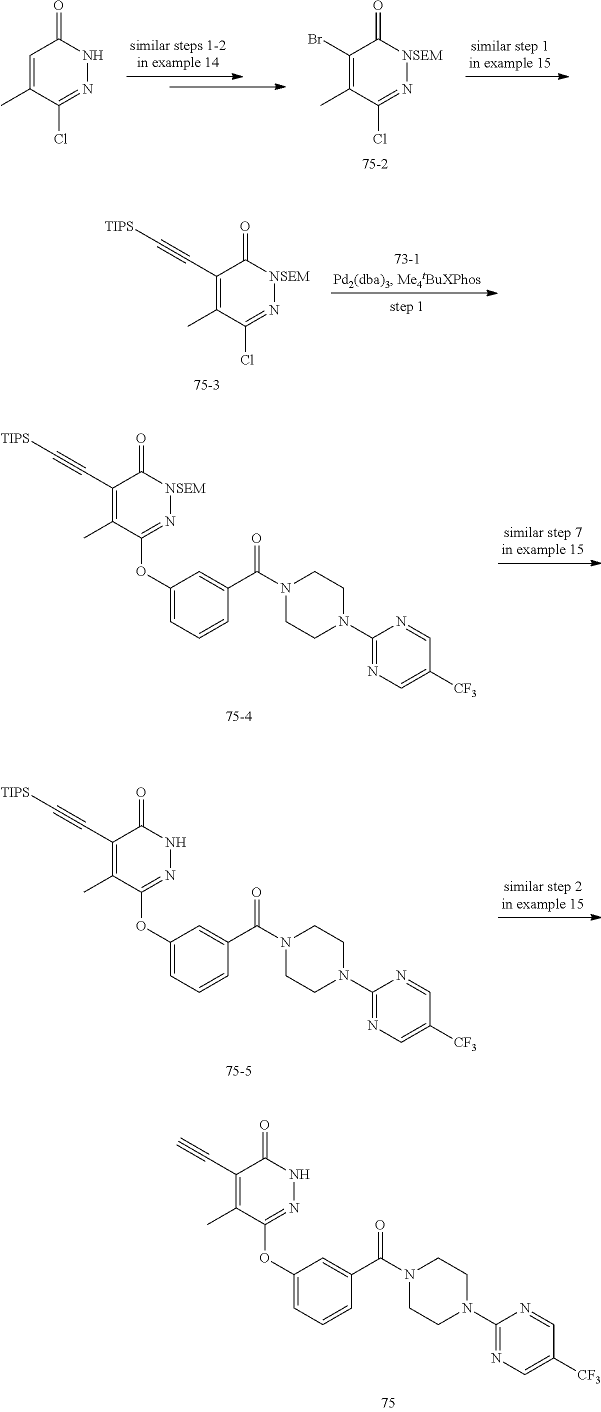

Compound 75-2 was prepared from 6-chloro-5-methylpyridazin-3 (2H)-one following the procedure for the synthesis of compound 36-2 in example 14.

Compound 75-3 was prepared from compound 75-2 following the procedure for the synthesis of compound 44-1 in example 15.

Step 1: To a solution of 75-3 (750 mg, 1.70 mmol) and 73-1 (599 mg, 1.70 mmol) in dioxane (5 mL) was added tris (dibenzylideneacetone) dipalladium (156 mg, 0.17 mmol), tetramethyl di-tBuXPhos (82 mg, 0.17 mmol) and Cs₂CO₃ (560 mg, 1.72 mmol). The mixture was stirred at 90° C. for 2 h. The reaction mixture was diluted with water and extracted with ethyl acetate. The combined organic layers were washed with water, dried over sodium sulfate, filtered, and concentrated. The residue was purified by column chromatography on silica gel (petroleum ether/ethyl acetate=5/1) to afford 75-4.

Compound 75-5 was prepared from compound 75-4 following the procedure for the synthesis of compound 36 in example 14.

Compound 75 was prepared from compound 75-5 following the procedure for the synthesis of compound 44-5 in example 15. LCMS (ESI, m z): [M+H]⁺=485.2; ¹H NMR (400 MHZ, methanol-d₄, ppm): δ 8.59 (s, 2H), 7.53 (t, J=8.1 Hz, 1H), 7.39-7.23 (m, 3H), 4.45 (s, 1H), 4.10-3.75 (m, 6H), 3.65-3.50 (m, 2H), 2.47 (s, 3H).

### Example 22 Synthesis of Compound 62

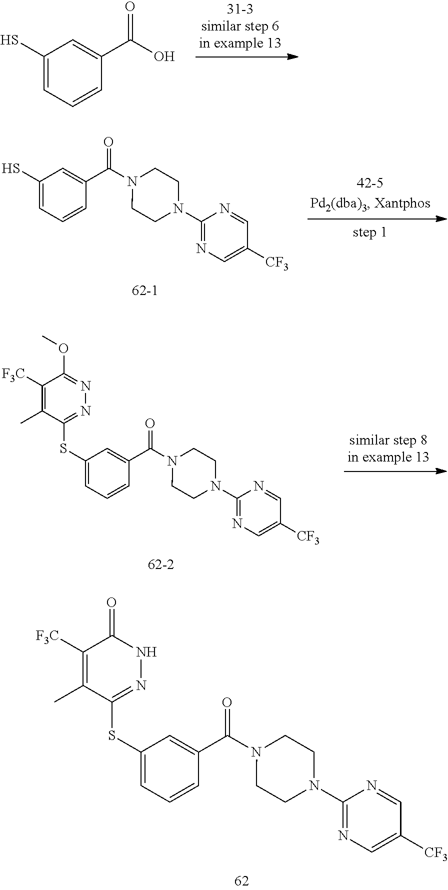 [SMILES: O=C(O)c1cccc(S)c1 ; O=C(c1cccc(S)c1)N1CCN(c2ncc(C(F)(F)F)cn2)CC1 ; COc1nnc(Sc2cccc(C(=O)N3CCN(c4ncc(C(F)(F)F)cn4)CC3)c2)c(C)c1C(F)(F)F ; Cc1c(Sc2cccc(C(=O)N3CCN(c4ncc(C(F)(F)F)cn4)CC3)c2)n[nH]c(=O)c1C(F)(F)F]

Compound 62-1 was prepared from 3-mercaptobenzoic acid following the procedure for the synthesis of compound 31-6 in example 13.

Step 1: A mixture of 42-5 (150 mg, 0.66 mmol), 62-1 (244 mg, 0.66 mmol), Xantphos (38 mg, 0.66 mmol), DIEA (257 mg, 2 mmol) and Pd₂ (dba)₃ (61 mg, 0.066 mmol) in dioxane (10 mL) was stirred at 100° C. for 16 h under N₂. The mixture was cooled and poured into water and extracted with ethyl acetate. The combined organic layers were dried over anhydrous sodium sulfate, filtered and concentrated. The residue was purified by column chromatography on silica gel (petroleum ether/ethyl acetate=3/1) to afford 62-2.

Compound 62 was prepared from 62-2 following the procedure for the synthesis of compound 31 in example 13. LCMS (ESI, m z): [M+H]⁺=545.2; ¹H NMR (400 MHZ, methanol-d₄, ppm): δ 8.59 (s, 2H), 7.65-7.47 (m, 4H), 4.10-3.74 (m, 6H), 3.62-3.44 (m, 2H), 2.52-2.49 (m, 3H).

### Example 23 Synthesis of Compound 70

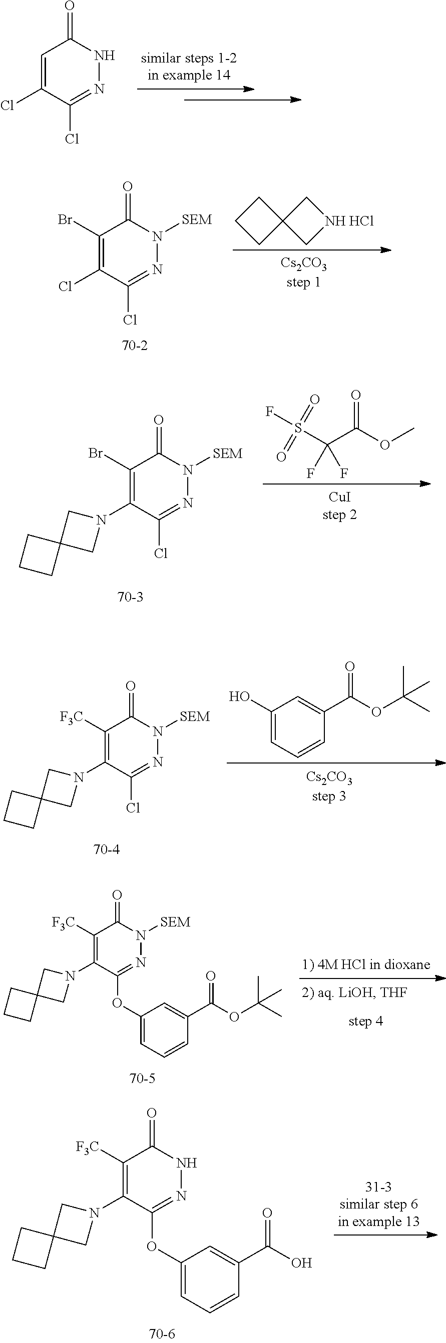

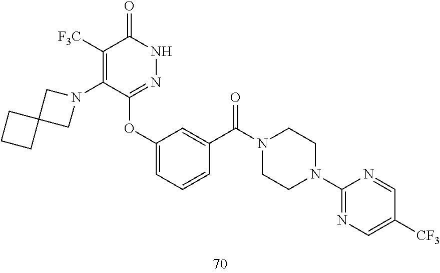 [SMILES: O=C(c1cccc(Oc2n[nH]c(=O)c(C(F)(F)F)c2N2CC3(CCC3)C2)c1)N1CCN(c2ncc(C(F)(F)F)cn2)CC1]

Compound 70-2 was prepared from 5,6-dichloropyridazin-3 (2H)-one following the procedure for the synthesis of compound 36-2 in example 14.

Step 1: To a solution of 70-2 (1 g, 2.67 mmol) in DMF (20 mL) were added 2-azaspiro[3.3]-heptane hydrochloride (428 mg, 3.21 mmol) and Cs₂CO₃ (2.61 g, 8.02 mmol). The reaction mixture was then stirred at 80° C. for 1 h. The mixture was cooled, diluted with water and extracted with ethyl acetate. The combined organic layers were washed with water, dried over Na₂SO₄, filtered and concentrated. The residue was purified by column chromatography on silica gel (petroleum ether/ethyl acetate=3/1) to afford 70-3.

Step 2: To a solution of 70-3 (500 mg, 1.15 mmol) in DMF (15 mL) were added methyl 2,2-difluoro-2-(fluor sulfonyl) acetate (663 mg, 3.45 mmol) and Cul (219 mg, 1.15 mmol). The reaction mixture was stirred at 110° C. under microwave condition for 0.5 h. The mixture was cooled, diluted with water and extracted with ethyl acetate. The combined organic layers were washed with water, dried over Na₂SO₄, filtered and concentrated. The residue was purified by column chromatography on silica gel (petroleum ether/ethyl acetate=3/1) to afford 70-4.

Step 3: To a solution of 70-4 (430 mg, 1.01 mmol) in DMF (8 mL) were added tert-butyl 3-hydroxy-benzoate (394 mg, 2.03 mmol) and Cs₂CO₃ (826 mg, 2.54 mmol). Then the reaction mixture was stirred at 80° C. for 8 h. The mixture was cooled, diluted with water and extracted with ethyl acetate. The combined organic layers were washed with water, dried over Na₂SO₄, filtered and concentrated. The residue was purified by column chromatography on silica gel (petroleum ether/ethyl acetate=2/1) to afford 70-5.

Step 4:70-5 (100 mg, 0.17 mmol) was dissolved in a solution of HCl in dioxane (4 M, 5 mL). The mixture was stirred at room temperature for 4 h. Then the solvent was removed under vacuum. The residue was dissolved in THF (8 mL) and H₂O (4 mL), then LiOH (21 mg, 0.50 mmol) was added. The reaction mixture was stirred at room temperature for 0.5 h. The pH was adjusted to 5-6 by addition of 1 M HCl and the mixture was extracted with ethyl acetate. The combined organic layers were washed with water, dried over Na₂SO₄, filtered and concentrated to afford 70-6 which was used directly in the next step without purification.

Compound 70 was prepared from 70-6 and 31-3 following the procedure for the synthesis of compound 31-6 in example 13. LCMS (ESI, m z): [M+H]⁺=610.0; ¹H NMR (400 MHZ, DMSO-d₆, ppm): δ 11.53 (s, 1H), 8.72 (s, 2H), 7.50 (t, J=8 Hz, 1H), 7.40-7.20 (m, 3H), 4.42 (s, 4H), 3.91-3.82 (m, 4H), 3.72-3.65 (m, 2H), 3.49-3.42 (m, 2H), 2.18-2.13 (m, 4H), 1.71-1.66 (m, 2H).

### Example 24 Synthesis of Compound 71

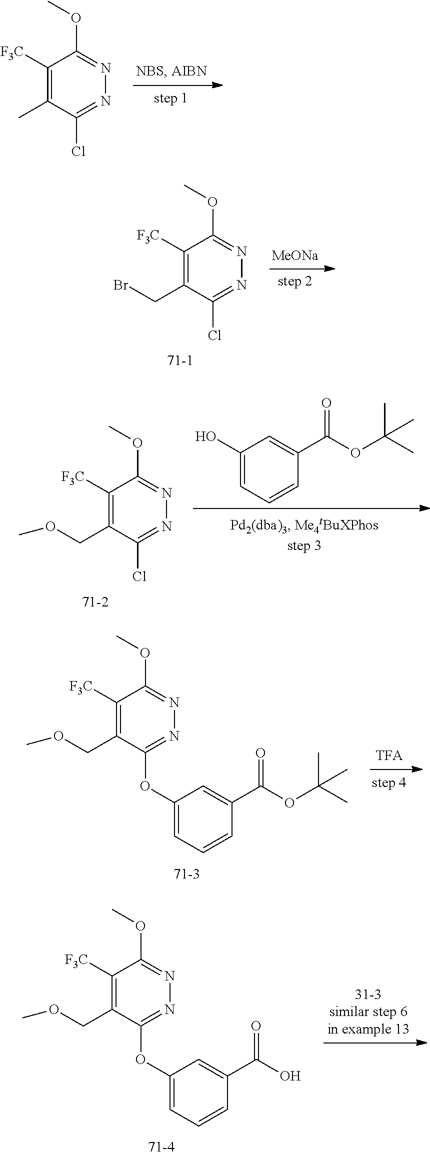

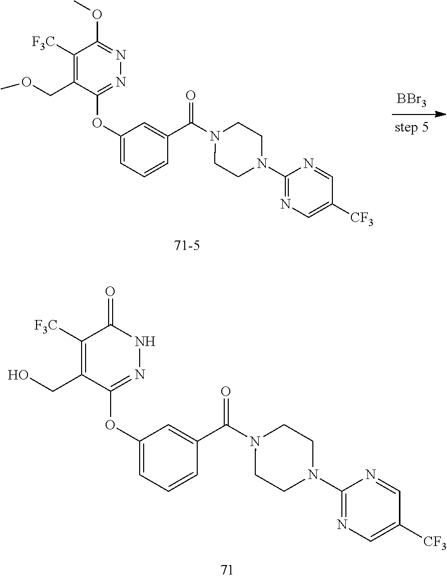 [SMILES: COCc1c(Oc2cccc(C(=O)N3CCN(c4ncc(C(F)(F)F)cn4)CC3)c2)nnc(OC)c1C(F)(F)F ; "BBr3" ; O=C(c1cccc(Oc2n[nH]c(=O)c(C(F)(F)F)c2CO)c1)N1CCN(c2ncc(C(F)(F)F)cn2)CC1]

Step 1: To a solution of 42-5 (5.0 g, 22.1 mmol) in CCl₄ (50 mL), was added NBS (5.1 g, 28.7 mmol) and AIBN(725 mg, 4.4 mmol). The resulting mixture was stirred at reflux overnight. The mixture was cooled, diluted with water and extracted with dichloromethane. The organic layer was dried over Na₂SO₄, filtered and concentrated, the residue was purified by reverse phase HPLC (acetonitrile with 0.05% of TFA in water: 5% to 70%) to afford 71-1.

Step 2: To a solution of 71-1 (507 mg, 1.00 mmol) in MeOH (5 mL) and THF (5 mL) was added MeONa (59 mg, 1.10 mmol) at 0° C. Then the mixture was stirred at room temperature for 1 h. The mixture was diluted with ethyl acetate and washed with water. The organic layer was dried over Na₂SO₄, filtered and concentrated. The residue was purified by column chromatography on silica gel (petroleum ether/ethyl acetate=20/1) to afford 71-2.

Step 3: To a solution of 71-2 (200 mg, 0.78 mmol) in dioxane (4 mL) were added tert-butyl 3-hydroxybenzoate (227 mg, 1.17 mmol), Me₄t-BuXphos (75 mg, 0.16 mmol), Pd₂ (dba)₃ (71 mg, 0.078 mmol) and Cs₂CO₃ (508 mg, 1.56 mmol). The mixture was stirred at 90° C. under N₂ for 2 h. The mixture was diluted with ethyl acetate and washed with water. The organic layer was dried over Na₂SO₄, filtered and concentrated. The residue was purified by column chromatography on silica gel (petroleum ether/ethyl acetate=2/1) to afford 71-3.

Step 4: To a solution of 71-3 (110 mg, 0.27 mmol) in dichloromethane (3 mL) was added TFA (3 mL) at 0° C. The mixture was stirred at room temperature for 1 h. After being concentrated to dryness, the residue was purified by reverse phase HPLC (acetonitrile with 0.05% of TFA in water: 5% to 80%) to afford 71-4.

Compound 71-5 was prepared from 71-4 and 31-3 following the procedure for the synthesis of compound 31-6 in example 13.

Step 5: To a solution of 71-5 (51 mg, 0.089 mmol) in DCM (10 mL) was added BBr₃ (223 mg, 0.89 mmol) under 0° C. The mixture was stirred at room temperature for 1 h, then poured into ice-water and extracted with dichloromethane. The combined organic layers were concentrated. The residue was purified by prep-HPLC (acetonitrile with 0.05% of TFA in water: 5% to 80%) to afford 71. LCMS (ESI, m z): [M+H]⁺=544.8; 1H NMR (400 MHZ, DMSO-d₆, ppm): δ 12.87 (s, 1H), 8.72 (s, 2H), 7.54-7.49 (m, 1H), 7.33-7.28 (m, 3H), 5.59 (brs, 1H), 4.57 (d, J=1.8 Hz, 2H), 3.98-3.81 (m, 4H), 3.80-3.51 (m, 4H).

### Example 25 Synthesis of Compound 76

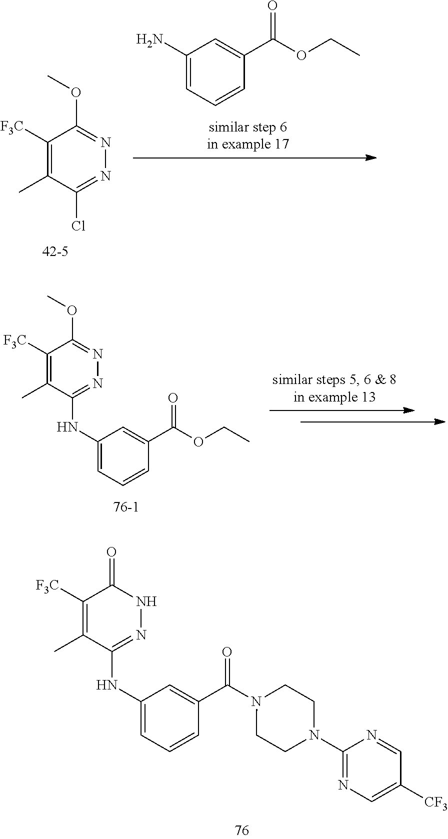 [SMILES: COc1nnc(Cl)c(C)c1C(F)(F)F ; CCOC(=O)c1cccc(N)c1 ; CCOC(=O)c1cccc(Nc2nnc(OC)c(C(F)(F)F)c2C)c1 ; Cc1c(Nc2cccc(C(=O)N3CCN(c4ncc(C(F)(F)F)cn4)CC3)c2)n[nH]c(=O)c1C(F)(F)F]

Compound 76-1 was prepared from 42-5 and ethyl 3-aminobenzoate following the procedure for the synthesis of compounds 42-6 in example 17.

Compound 76 was prepared from 76-1 following the procedure for the synthesis of compound 31 in example 13 as a 3.5 eq of TFA salt. LCMS (ESI, m z): [M+H]⁺=528.1; 1H NMR (400 MHZ, DMSO-d₆, ppm): § 12.66 (s, 1H), 8.72 (s, 2H), 8.12 (s, 1H), 7.48-7.29 (m, 3H), 6.97 (d, J=7.4 Hz, 1H), 3.95-3.80 (m, 4H), 3.75-3.45 (m, 4H), 2.42-2.38 (m, 3H). ¹⁹F NMR (376 MHz, DMSO-d₆, ppm): δ-57.19 (3F), -59.33 (3F).

### Example 26 Synthesis of Compounds 80 and 108

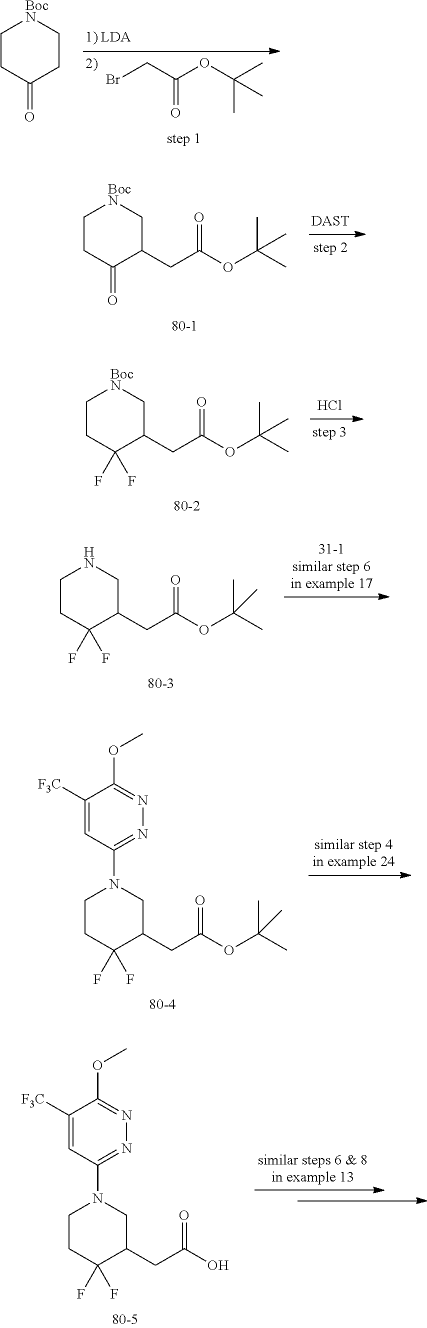

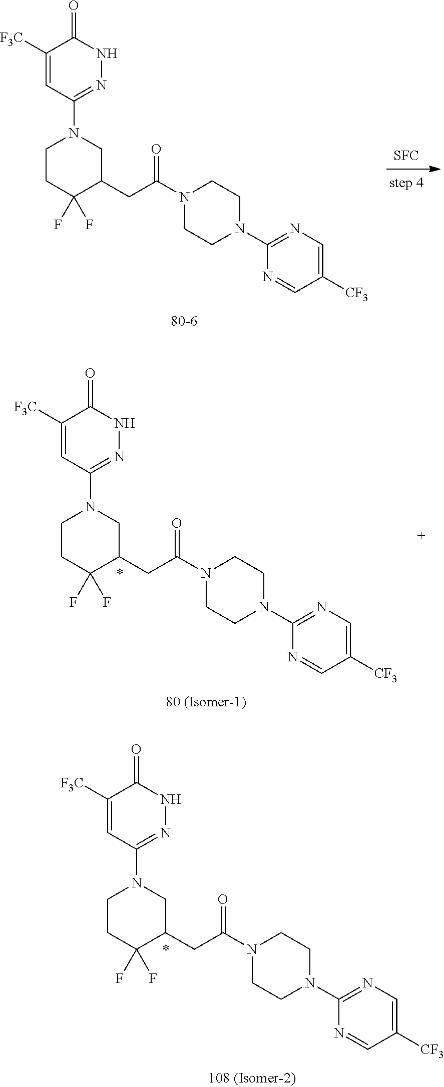 [SMILES: O=C(CC1CN(c2cc(C(F)(F)F)c(=O)[nH]n2)CCC1(F)F)N1CCN(c2ncc(C(F)(F)F)cn2)CC1 ; "SFC" ; O=C(CC1CN(c2cc(C(F)(F)F)c(=O)[nH]n2)CCC1(F)F)N1CCN(c2ncc(C(F)(F)F)cn2)CC1 ; O=C(CC1CN(c2cc(C(F)(F)F)c(=O)[nH]n2)CCC1(F)F)N1CCN(c2ncc(C(F)(F)F)cn2)CC1]

Step 1: To a solution of tert-butyl 4-oxopiperidine-1-carboxylate (50 g, 250.94 mmol) in THF (400 mL) was added 2M LDA solution in THF (200 mL, 401.50 mmol) dropwise at −70° C., then a solution of tert-butyl 2-bromoacetate (48.95 g, 250.94 mmol) in THF (100 mL) was added dropwise, followed by addition of HMPA (19.8 g, 110.41 mmol). The resulting mixture was warmed up slowly to room temperature and stirred for 12 h. After being quenched with sat. aq. NH₄Cl solution, the mixture was concentrated to remove the organic solvent. The aqueous phase was extracted with ethyl acetate. The organic layer was dried over Na₂SO₄, filtered and concentrated. The residue was purified by column chromatography on silica gel (petroleum ether/ethyl acetate=9/1) to afford 80-1.

Step 2: To a solution of 80-1 (15 g, 47.86 mmol) in dichloromethane (300 mL) was added DAST (23.1 g, 143.59 mmol) dropwise at 0° C. The mixture was warmed to room temperature and stirred for 16 h. The reaction was quenched with sat. aq. NaHCO₃ solution. The aqueous phase was separated and extracted with dichloromethane. The combined organic layers were washed with brine, dried over anhydrous Na₂SO₄, filtered and concentrated. The residue was purified by column chromatography on silica gel (petroleum ether/ethyl acetate=12/1) to afford 80-2.

Step 3: To a solution of 80-2 (2 g, 5.96 mmol) in dioxane (8 mL) was added 4 M HCl solution in dioxane (2 mL, 8.0 mmol). Then the reaction mixture was stirred at room temperature for 5 h. The pH was adjusted to ca. 6 by addition of sat. aq. NaHCO₃ solution and extracted with ethyl acetate. The combined organic layers were dried over Na₂SO₄, filtered and concentrated. The residue was purified by column chromatography on silica gel (petroleum ether to ethyl acetate) to afford 80-3.

Compound 80-4 was prepared from 80-3 and 31-3 following the procedure for the synthesis of compounds 42-6 in example 17.

Compound 80-5 was prepared from 80-4 following the procedure for the synthesis of compounds 71-4 in example 24.

Compound 80-6 was prepared from 80-5 following the procedure for the synthesis of compound 31 in example 13.

80-6 (200 mg) was separated by SFC (column: DAICEL CHIRALPAK®IG, MeOH (+0.1% 7.0 mol/L ammonia in MeOH)/CO₂=60/40) to afford 80 (51.8 mg) and 108 (63.6 mg) respectively. 80: SFC analysis: >99% ee; retention time: 1.20 min; column: DAICEL CHIRALPAK®IG, MeOH (0.1% of DEA) in CO₂, 40%; pressure: 100 bar; flow rate: 1.8 mL/min. LCMS (ESI, m/z): [M+H]⁺=556.0; ¹H NMR (400 MHZ, DMSO-d₆): δ 12.73 (s, 1H), 8.72 (s, 2H), 7.94 (s, 1H), 3.85-3.80 (m, 6H), 3.58-3.56 (m, 4H), 3.11-3.04 (m, 1H), 2.85-2.79 (m, 1H), 2.75-2.71 (m, 1H), 2.65-2.56 (m, 1H), 2.43-2.35 (m, 1H), 2.15-2.00 (m, 2H). ¹⁹F NMR (376 MHz, DMSO-d₆, ppm): δ-59.33, -65.80. 108:0.7 eq TFA salt; SFC analysis: >99% ee; retention time: 2.22 min; column: DAICEL CHIRALPAK®IG, MeOH (0.1% of DEA) in CO₂, 40%; pressure: 100 bar; flow rate: 1.8 mL/min. LCMS (ESI, m/z): [M+H]⁺=556.0; ¹H NMR (400 MHZ, DMSO-d₆): δ 12.82-12.63 (m, 1H), 8.71 (s, 2H), 7.94 (s, 1H), 3.85-3.81 (m, 6H), 3.65-3.50 (m, 4H), 3.11-3.04 (m, 1H), 2.85-2.79 (m, 1H), 2.75-2.65 (m, 1H), 2.61-2.56 (m, 1H), 2.50-2.30 (m, 1H), 2.20-2.00 (m, 2H). ¹⁹F NMR (376 MHZ, DMSO-d₆, ppm): δ-59.33, -65.80.

### Example 27 Synthesis of Compound 87

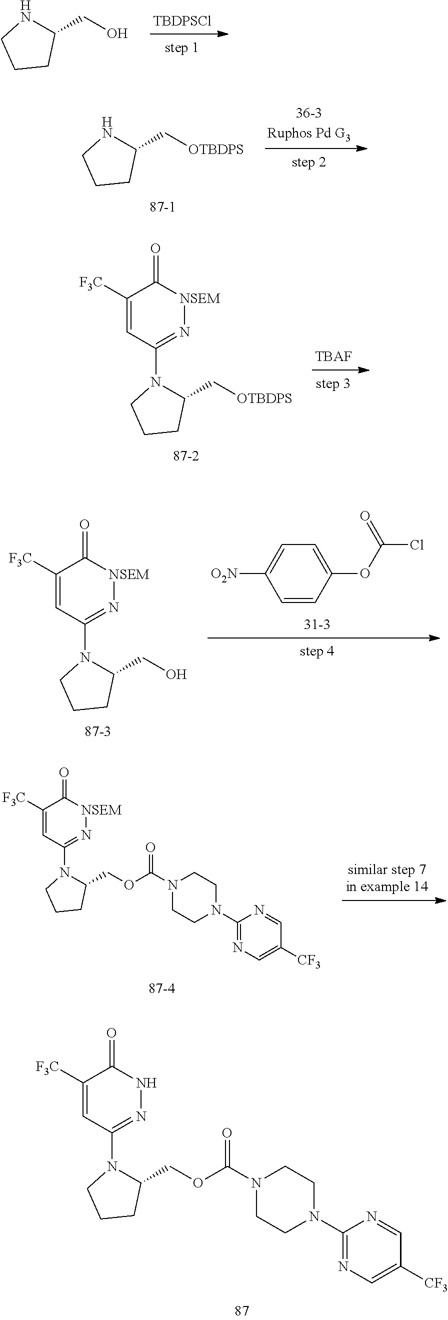

Step 1: To a solution of(S)-pyrrolidin-2-ylmethanol (1.41 g, 13.98 mmol) and imidazole (2.38 g, 34.94 mmol) in dichloromethane (40 mL) was added TBDPSCI (5.76 g, 20.96 mmol) at 0° C. Then the mixture was stirred at 0° C. for 3 h. The mixture was poured into water. The resulting solution was extracted with dichloromethane. The combined organic layers were dried over anhydrous sodium sulfate, filtered and concentrated. The residue was purified by column chromatography on silica gel (dichloromethane/methanol=10/1) to afford 87-1.

Step 2: A mixture of 36-3 (2 g, 6.09 mmol), 87-1 (4.13 g, 12.2 mmol), Cs₂CO₃ (3.96 g, 12.2 mmol) and RuPhos Pd G³ (510 mg, 0.61 mmol) in dioxane (20 mL) was stirred at 110° C. for 16 h under N₂. The mixture was poured into water. The resulting solution was extracted with ethyl acetate. The combined organic layers were dried over anhydrous sodium sulfate, filtered and concentrated. The residue was purified by column chromatography on silica gel (petroleum ether/ethyl acetate=10/1) to afford 87-2.

Step 3: To a solution of 87-2 (2.00 g, 3.17 mmol) in THF (20 mL) was added a solution of TBAF in THF (1 M, 5 mL) at room temperature. The mixture was stirred at room temperature for 5 h. The mixture was concentrated. The residue was purified by column chromatography on silica gel (petroleum ether/ethyl acetate=5/1) to afford 87-3.

Step 4: To a solution of 87-3 (500 mg, 1.27 mmol) in dichloromethane (20 mL) was added pyridine (503 mg, 6.35 mmol) and 4-nitrophenyl carbonochloridate (1.11 g, 5.08 mmol). The mixture was stirred at room temperature for 6 h under N₂. Then 31-3 (590 mg, 2.5 mmol) in DMF (10 mL) was added. The mixture was stirred at 50° C. for 16 h. The mixture was poured into water. The resulting solution was extracted with ethyl acetate. The combined organic layers were dried over anhydrous sodium sulfate, filtered and concentrated. The residue was purified by reverse phase HPLC (acetonitrile with 0.1% of FA in water: 5% to 95%) to afford 87-4.

Compound 87 was prepared from 87-4 following the procedure for the synthesis of compound 36 in example 14. LCMS (ESI, m z): [M+H]=522.3; ¹H NMR (400 MHZ, methanol-d₄, ppm): δ 8.58 (s, 2H), 7.75 (s, 1H), 4.29-4.17 (m, 2H), 4.11 (dd, J=10.8, 6.4 Hz, 1H), 3.94-3.87 (m, 4H), 3.59-3.51 (m, 5H), 3.39-3.33 (m, 1H), 2.13-1.98 (m, 4H).

### Example 28 Synthesis of Compounds 81 and 117

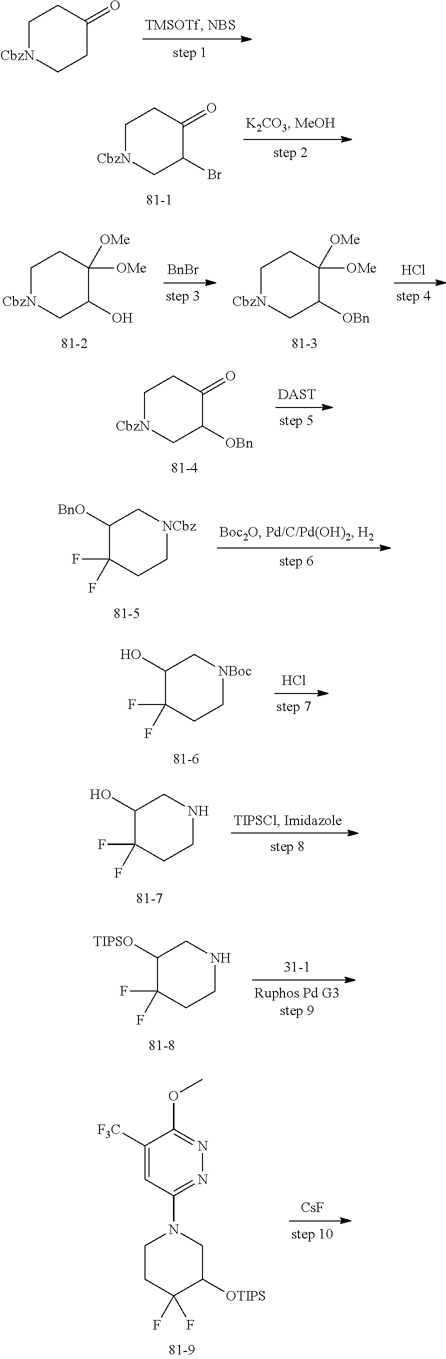

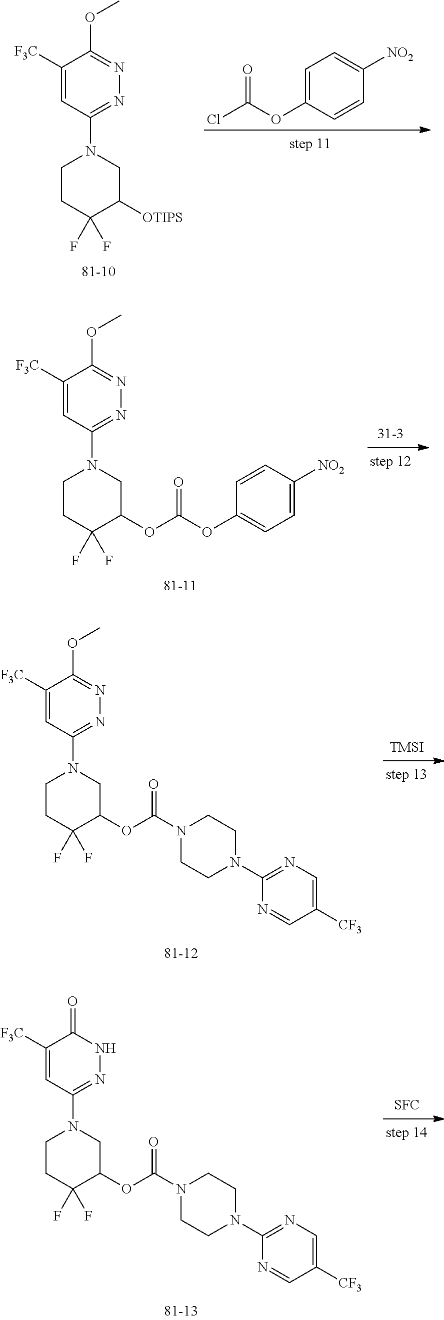

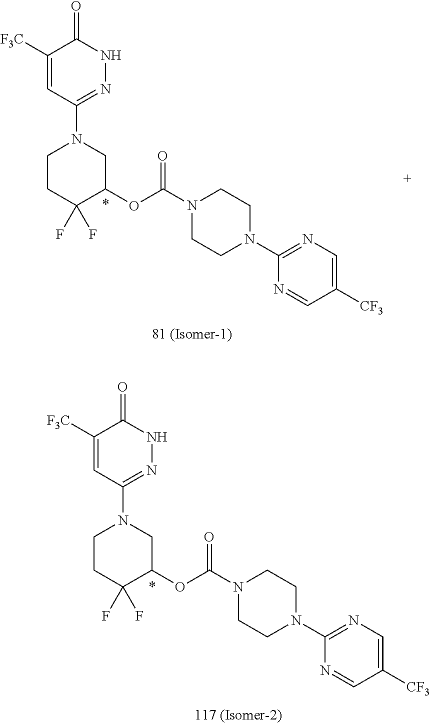 [SMILES: O=C(OC1CN(c2cc(C(F)(F)F)c(=O)[nH]n2)CCC1(F)F)N1CCN(c2ncc(C(F)(F)F)cn2)CC1 ; O=C(OC1CN(c2cc(C(F)(F)F)c(=O)[nH]n2)CCC1(F)F)N1CCN(c2ncc(C(F)(F)F)cn2)CC1]

Step 1: To a solution of benzyl 4-oxopiperidine-1-carboxylate (100 g, 428 mmol) in dichloromethane (1 L) was added DIEA (89 mL, 535 mmol) at −10° C., followed by addition of TMSOTf (95 mL, 514 mmol) dropwise. The reaction mixture was stirred at −10-0° C. for 0.5 h. Then NBS (77.8 g, 437 mmol) was added in portions and the resulting mixture was stirred at room temperature for 12 h. The mixture was quenched with aq. NaHCO₃ solution and the organic layer was washed with brine, dried over Na₂SO₄, filtered and concentrated. The residue was purified by silica gel column chromatography (petroleum ether/ethyl acetate=50/1 to 10/1) to afford 81-1.

Step 2: To a solution of 81-1 (164 g, 525 mmol) in MeOH (1.5 L) was added K₂CO₃ (145 g, 1.05 mol) in five portions at room temperature and the mixture was stirred at room temperature for 20 h. The mixture was diluted with ethyl acetate, filtered and the filtrate was washed with water, brine, dried over Na₂SO₄, filtered and concentration to afford 81-2 which was used directly in the next step without further purification.

Step 3: At 0° C., to a suspension of 60% w.t NaH in mineral oil (10.6 g, 440.19 mmol) in THF (200 mL) was added a solution 81-2 (130 g, 440.19 mmol) in THF (200 mL) dropwise. The mixture was stirred at room temperature for 20 min, then (bromomethyl) benzene (52.65 mL, 440.19 mmol) was added dropwise. The mixture was stirred at 70° C. for 16 h. The mixture was cooled, diluted with water and extracted with ethyl acetate. The combined organic layers were washed with brine, dried over Na₂SO₄, filtered and concentrated. The residue was purified by silica gel column chromatography (petroleum ether/ethyl acetate=50/1 to 10/1) to afford 81-3.

Step 4: To a solution of 81-3 (50 g, 129.72 mmol) in THF (400 mL) was added 3N HCl (433 mL, 1.3 mol). The mixture was stirred at 50° C. for 6 h. The mixture was cooled, basified with NaHCO₃ solution and extracted with ethyl acetate. The combined organic layers were washed with water and brine, dried over Na₂SO₄, filtered and concentrated to afford 81-4 which was used directly in the next step without further purification.

Step 5: To a solution of 81-4 (15 g, 44.20 mmol) in dichloromethane (200 mL) was added DAST (17.8 g, 110.49 mmol) at −20° C. The mixture was stirred at 0° C. for 2 h. The reaction was quenched with water. The organic layer was washed with brine, dried over Na₂SO₄, filtered and concentrated. The residue was purified by silica gel column chromatography (petroleum ether/ethyl acetate=10/1) to afford 81-5.

Step 6: To a solution of 81-5 (8 g, 22.14 mmol) and Boc₂O (8.64 g, 40 mmol) in tert-butanol (100 mL) and MeOH (140 mL) were added 20% w.t Pd/C (1 g) and 20% w.t Pd(OH)₂ (1 g). The mixture was hydrogenated under 1 atm hydrogen atmosphere at 70° C. for 24 h. Then, additional Pd/C (1 g) and Pd(OH)₂ (1 g) were added and the mixture was stirred under 1 atm hydrogen atmosphere for another 24 h at 70° C. The mixture was cooled, filtered and the filtrate was concentrated. The residue was purified by silica gel column chromatography (methanol/dichloromethane=3/97) to afford 81-6.

Step 7: To a solution of 81-6 (700 mg, 2.95 mmol) in MeOH (3 mL) was added 4N HCl solution in 1,4-dioxane (4 mL). The mixture was stirred at room temperature for 1 h and concentrated to afford 81-7 which was used directly in the next step without further purification.

Step 8: To a solution of 81-7 (350 mg, 2.55 mmol) in acetonitrile (10 mL) were added TIPSCI (740 mg, 3.83 mmol) and imidazole (521 mg, 7.66 mmol). The mixture was stirred at 60° C. for 10 h. After being cooled to room temperature, water was added and the mixture was extracted with ethyl acetate. The combined organic layers were washed with brine, dried over Na₂SO₄, filtered and concentrated. The residue was purified by silica gel column chromatography (petroleum ether/ethyl acetate=20/1 to 1/1) to afford 81-8.

Step 9: To a solution of 81-8 (300 mg, 1.02 mmol) and 31-1 (220 mg, 1.02 mmol) in dioxane (5 mL) were added Cs₂CO₃ (666 mg, 2.04 mmol) and RuPhos Pd G³ (171 mg, 0.20 mmol). The reaction mixture was stirred at 110° C. for 2 h under N₂. After being cooled to room temperature, water was added and the mixture was extracted with ethyl acetate. The combined organic layers were washed with brine, dried over Na₂SO₄, filtered and concentrated. The residue was purified by silica gel column chromatography (petroleum ether/ethyl acetate=20/1 to 3/1) to afford 81-9.

Step 10: To a solution of 81-9 (410 mg, 0.87 mmol) in CH₃CN(10 mL) was added CsF (265 mg, 1.75 mmol). The mixture was stirred at 60° C. for 2 h. After being cooled to room temperature, the mixture was filtered and the filtrate was concentrated. The residue was purified by silica gel column chromatography (petroleum ether/ethyl acetate=20/1 to 1/4) to afford 81-10.

Step 11: At 0° C., to a solution of 81-10 (210 mg, 0.67 mmol) and 4-nitrophenyl chloroformate (205 mg, 1.0 mmol) in THF (5 mL) was added TEA (0.19 mL, 1.34 mmol). The mixture was stirred at room temperature for 1 h. After being quenched with water, the mixture was extracted with ethyl acetate. The combined organic layers were washed with water and brine, dried over Na₂SO₄, filtered and concentrated. The residue was purified by silica gel column chromatography (petroleum ether/ethyl acetate=10/1) to afford 81-11.

Step 12: To a solution of 81-11 (200 mg, 0.42 mmol) and 31-3 (110 mg, 0.46 mmol) in CH₃CN(5 mL) was added K₂CO₃ (116 mg, 0.84 mmol) and the mixture was stirred at 80° C. for 1 h. After being cooled to room temperature, the mixture was filtered and the filtrate was concentrated. The residue was purified by silica gel column (petroleum ether/ethyl acetate=20/1 to 3/2) to afford 81-12.

Step 13: To a solution of 81-12 (150 mg, 0.26 mmol) in CH₃CN(8 mL) was added TMSI (184 mg, 1.31 mmol). The mixture was stirred at 60° C. for 1 h. After being cooled to room temperature, the mixture was quenched with Na₂SO₃ solution. Water was added and the mixture was extracted with ethyl acetate. The combined organic layers were washed with brine, dried over Na₂SO₄, filtered and concentrated. The residue was purified by reverse phase HPLC (acetonitrile with 0.05% of TFA in water: 5% to 55%) to afford 81-13.

Step 14:81-13 (90 mg) was purified by SFC (column: DAICEL CHIRALPAK®IG, MeOH (+0.1% 7.0 mol/L ammonia in MeOH)/CO₂=60/40) to afford 117 (30.2 mg) and 81 (30.5 mg) respectively.

117: SFC analysis: >99% ee; retention time: 2.50 min; column: DAICEL CHIRALPAK®IG, MeOH (0.1% of DEA) in CO₂, 5% to 40%; pressure: 100 bar; flow rate: 1.5 mL/min. LCMS (ESI, m/z): [M+H]⁺=558.4; ¹H NMR (400 MHZ, DMSO-d₆, ppm)δ 12.77 (s, 1H), 8.74 (s, 2H), 8.04 (s, 1H), 5.03-4.98 (m, 1H), 4.00-3.19 (m, 12H), 2.34-2.08 (m, 2H).

81: SFC analysis: 97.1% ee; retention time: 2.84 min; column: DAICEL CHIRALPAK®IG, MeOH (0.1% of DEA) in CO₂, 5% to 40%; pressure: 100 bar; flow rate: 1.5 mL/min. LCMS (ESI, m/z): [M+H]⁺=558.4; ¹H NMR (400 MHZ, DMSO-d₆, ppm)δ 12.79 (s, 1H), 8.74 (s, 2H), 8.04 (s, 1H), 5.04-4.98 (m, 1H), 4.03-3.18 (m, 12H), 2.33-2.07 (m, 2H).

### Example 29 Synthesis of Compound 183

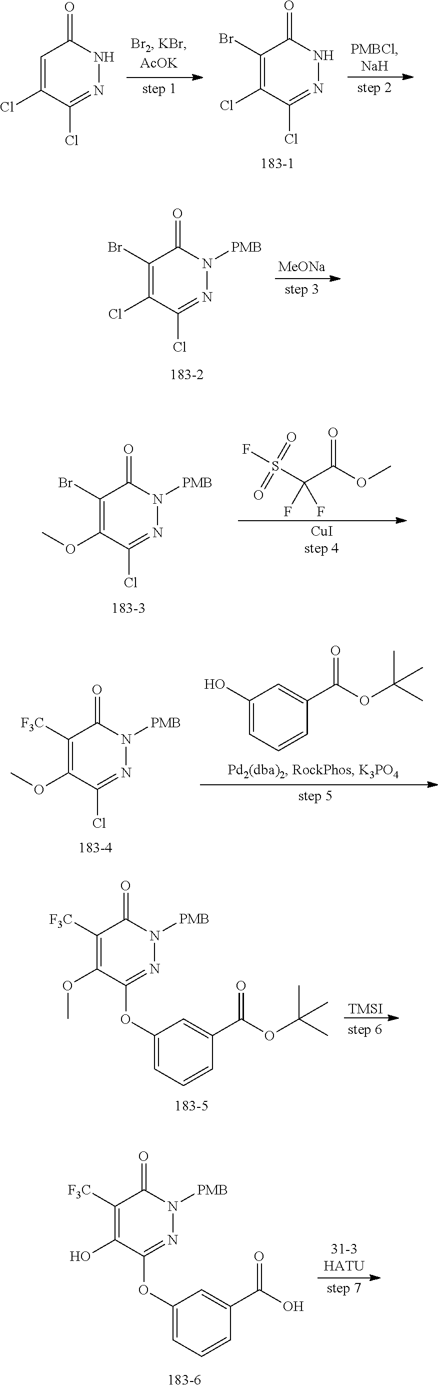

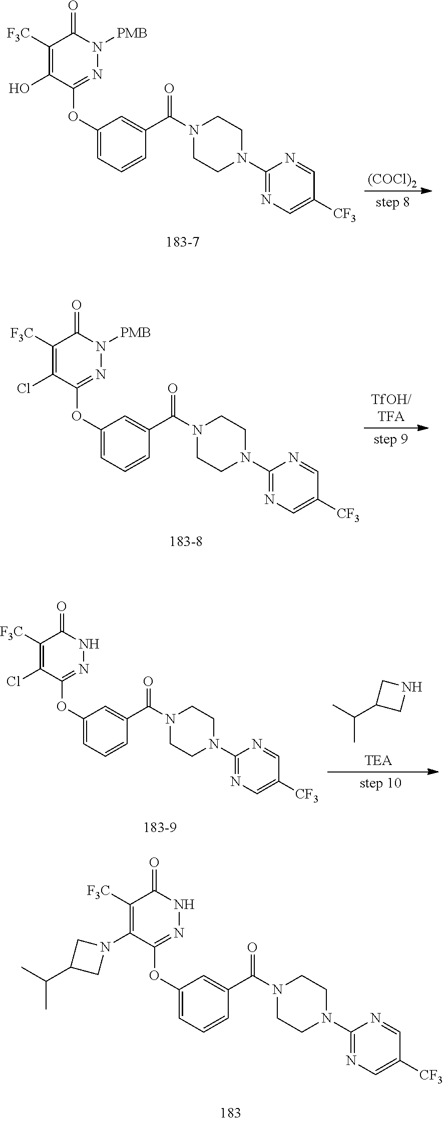

Step 1: To a mixture of 5,6-dichloro-2,3-dihydropyridazin-3-one (20 g, 165 mmol), KBr (43.3 g, 364 mmol) and AcOK (17.8 g, 182 mmol) in H₂O (120 mL) was added Br₂ (18.6 mL, 364 mmol). The mixture was stirred at 100° C. for 2 h. The mixture was cooled, filtered and the filter cake was washed with saturated aqueous solution of Na₂SO₃ (200 mL) and water (200 mL) successively. The filter cake was dried to afford 183-1.

Step 2: At 0° C., to a mixture of 183-1 (26 g, 106.6 mmol) in anhydrous DMF (220 mL) was added NaH (60% w.t, 8.5 g, 213.2 mmol) in portions. The mixture was stirred at this temperature for 30 min. Then 1-(chloromethyl)-4-methoxybenzene (28.9 mL, 213.2 mmol) was added dropwise and the resulting mixture was stirred at room temperature for 1.5 h. After being quenched with H₂O, the mixture was extracted with ethyl acetate. The combined organic layers were washed with brine, dried over anhydrous Na₂SO₄, filtered and concentrated. The residue was purified by column chromatography on silica gel (petroleum ether/ethyl acetate=20/1 to 4/1) to afford 183-2.

Step 3: To a mixture of 183-2 (10.1 g, 27.7 mmol) in methanol (100 mL) was added sodium methanolate (4.5 g, 83.2 mmol). The mixture was stirred at room temperature for 16 h. The mixture was quenched by addition of water and the aqueous layer was extracted with ethyl acetate. The combined organic layers were washed with brine, dried over anhydrous Na₂SO₄, filtered and concentrated. The residue was purified by column chromatography on silica gel (petroleum ether/ethyl acetate=6/1) to afford 183-3.

Step 4: To a solution of 183-3 (7 g, 19.5 mmol) in DMF (70 mL) was added methyl 2,2-difluoro-2-(fluorosulfonyl) acetate (11.22 g, 58.4 mmol) and Cul (3.7 g, 19.5 mmol). The reaction mixture was degassed by bubbling nitrogen for 5 min. Then the mixture was stirred at 100° C. for 2 h. The mixture was poured into water and extracted with ethyl acetate. The combined organic layers were washed with brine, dried over anhydrous Na₂SO₄, filtered and concentrated. The residue was purified by column chromatography on silica gel (petroleum ether/ethyl acetate=4/1) to afford 183-4.

Step 5: To a solution of 183-4 (4 g, 11.5 mmol) in toluene (100 mL) were added tert-butyl 3-hydroxybenzoate (4.46 g, 22.9 mmol), Pd₂ (dba)₃ (3.2 g, 3.4 mmol), t-BuXPhos (243 mg, 0.57 mmol) and Cs₂CO₃ (9.3 g, 28.7 mmol). The mixture was degassed by bubbling nitrogen for 5 min. Then the mixture was stirred at 80° C. for 2 h. The mixture was cooled and concentrated. The residue was purified by column chromatography on silica gel (petroleum ether/ethyl acetate=4/1) to afford 183-5.

Step 6: To a solution of 183-5 (1 g, 1.97 mmol) in acetonitrile (20 mL) was added iodotrimethylsilane (0.56 mL, 3.95 mmol). Then the mixture was stirred at 50° C. for 1 h. The mixture was cooled, quenched with water, and extracted with ethyl acetate. The combined organic layers were washed with brine, dried over anhydrous Na₂SO₄, filtered, and concentrated to afford crude 183-6 which was used directly in the next step.

Step 7: To a solution of 183-6 (880 mg, 1.51 mmol) and 31-3 (406 mg, 1.51 mmol) in dichloromethane (20 mL) were added DIPEA (0.75 mL, 4.54 mmol) and HATU (575 mg, 1.51 mmol). The mixture was stirred at room temperature for 20 min, quenched with H₂O and extracted with dichloromethane. The combined organic layers were washed with brine, dried over anhydrous Na₂SO₄, filtered and concentrated. The residue was purified by prep-HPLC (acetonitrile with 0.05% of TFA in water: 10% to 70%) to afford 183-7.

Step 8: To a solution of 183-7 (820 mg, 1.26 mmol) in DMF (12 mL) was added oxalic dichloride (0.21 mL, 2.52 mmol) dropwise at 0° C. The mixture was stirred at room temperature for 2 h. The reaction was quenched with H₂O. The precipitate was filtered and dried to afford 183-8 which was used directly in the next step.

Step 9: To a solution of 183-8 (800 mg, 1.20 mmol) in TFA (10 mL) was added TfOH (1 mL). The mixture was stirred at room temperature overnight. The solvent was removed under vacuum and the residue was diluted with acetonitrile (15 mL). The pH was adjusted to 9 by addition of saturated aqueous solution of NaHCO₃. The mixture was extracted with ethyl acetate. The combined organic layers were washed with brine, dried over anhydrous Na₂SO₄, filtered and concentrated. The residue was purified by column chromatography on silica gel (dichloromethane/methanol=10/1) to afford 183-9.

Step 10: To a mixture of 183-9 (100 mg, 0.18 mmol) and 3-(propan-2-yl) azetidine (27 mg, 0.27 mmol) in acetonitrile (1 mL) was added TEA (0.076 mL, 0.55 mmol). Then the mixture was stirred at room temperature for 1 h. The mixture was concentrated and the residue was purified by column chromatography on silica gel (dichloromethane/methanol=10/1) to afford 183. LCMS (ESI, m z): [M+H]=612.6. ¹H NMR (400 MHZ, CDCl₃, ppm): δ 10.04 (s, 1H), 8.52 (s, 2H), 7.55-7.45 (m, 1H), 7.37-7.28 (m, 1H), 7.25-6.92 (m, 2H), 4.63-4.22 (m, 4H), 4.10-3.52 (m, 8H), 2.50-1.92 (m, 2H), 0.94-0.83 (m, 6H); ¹⁹F NMR (376 MHZ, CDCl₃, ppm): δ-51.64 (3F), -61.10 (3F).

### Example 30 Synthesis of Compound 175

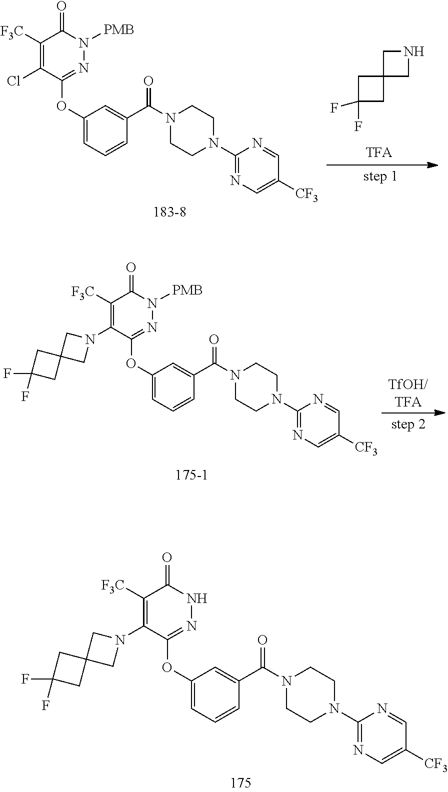

Step 1: To a solution of 183-8 (60 mg, 0.090 mmol) in acetonitrile (3 mL) were added TEA (27 mg, 0.27 mmol), and 6,6-difluoro-2-azaspiro[3.3]heptane trifluoroacetate (30 mg, 0.13 mmol). The mixture was stirred at room temperature for 30 min. The mixture was diluted with ethyl acetate and water. The organic layer was separated, washed with brine, and concentrated. The residue was purified by silica gel column chromatography (petroleum ether/ethyl acetate=3/1) to afford 175-1.

Step 2: To a solution of 175-1 (45 mg, 0.06 mmol) in TFA (5 mL) was added trifluoromethanesulfonic acid (0.5 mL). The mixture was stirred at room temperature for 18 h. The reaction was diluted with ethyl acetate and water. The organic layer was separated, washed with bine, and concentrated. The residue was purified by reverse phase HPLC (acetonitrile with 0.05% of TFA in water: 10% to 50%) to afford 175 as 1.9 eq of TFA salt. LCMS (ESI, m z): [M+H]⁺=646.5; ¹HNMR (400 MHZ, DMSO-d₆, ppm): δ 11.62 (s, 1H), 8.75 (s, 2H), 7.53-7.51 (m, 1H), 7.37-7.30 (m, 3H), 4.58 (s, 4H), 4.00-3.75 (m, 4H), 3.70-3.60 (m, 2H), 3.53-3.48 (m, 2H), 2.95-2.81 (m, 4H). ¹⁹FNMR (376 MHz, DMSO-d₆, ppm): δ -51.42 (3F), -59.29 (3F), -91.10 (2F).

### Example 31 Synthesis of Compound 236

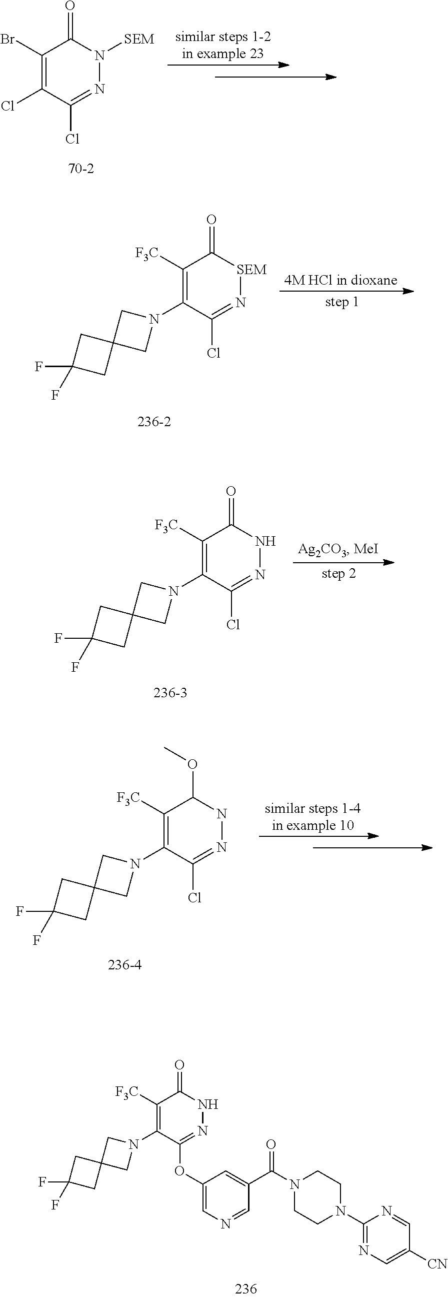

Compound 236-2 was prepared from compound 70-2 following the procedure for the synthesis of compound 70-4 in example 23.

Step 1: A mixture of 236-2 (3.2 g, 6.96 mmol) and 4M HCl in 1,4-dioxane (32 mL) was stirred at room temperature for 18 h. The mixture was concentrated to afford 236-3 which was used directly in the next step.

Step 2: To a solution of 236-3 (2 g, 6.07 mmol) in 1,4-dioxane (20 mL) were added Ag₂CO₃ (2.5 g, 9.10 mmol) and Mel (0.9 g, 6.07 mmol) and the mixture was stirred at 40° C. for 12 h. The mixture was diluted with ethyl acetate and brine. The organic layer was separated and concentrated. The residue was purified by silica gel column chromatography (petroleum ether/ethyl acetate=3/1) to afford 236-4.

Compound 236 was prepared as a 2.39 eq of TFA salt from compound 236-4 following the procedure for the synthesis of compound 20 in example 10. LCMS (ESI, m z): [M+H]⁺=604.5; ¹H NMR (400 MHZ, DMSO-d₆, ppm): δ 11.67 (s, 1H), 8.81 (s, 2H), 8.65 (d, J=2.6 Hz, 1H), 8.55 (s, 1H), 7.93-7.89 (m, 1H), 4.60 (s, 4H), 4.01-3.88 (m, 4H), 3.80-3.70 (m, 4H), 2.89 (t, J=12.5 Hz, 4H). ¹⁹F NMR (376 MHz, DMSO-d₆)8-51.42 (3F), -91.01 (2F).

### Example 32 Synthesis of Compound 217

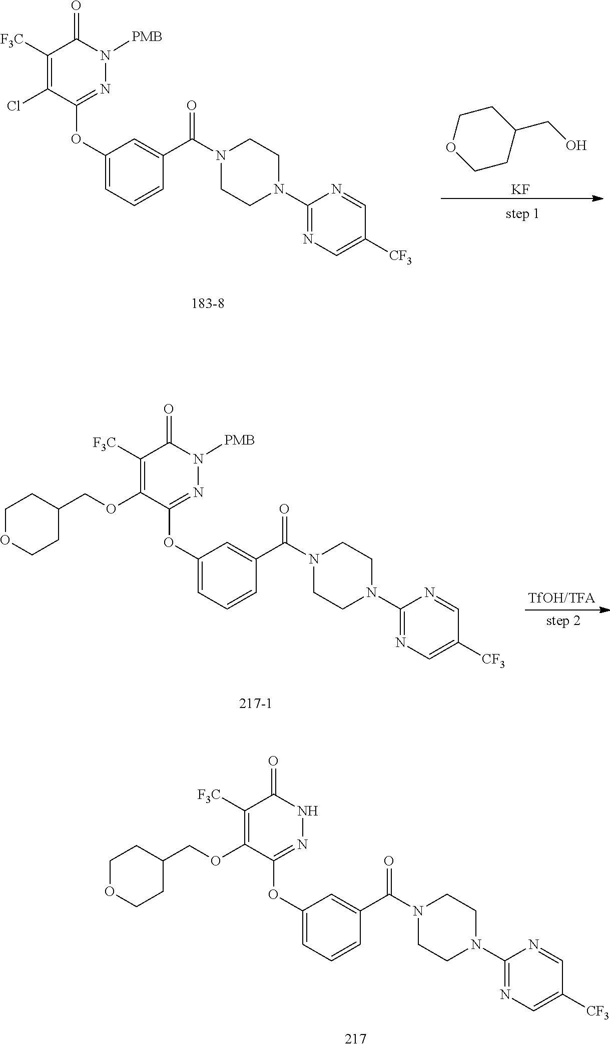

Step 1: To a solution of 183-8 (60 mg, 0.090 mmol) in DMF (2 mL) were added (oxan-4-yl) methanol (52 mg, 0.45 mmol) and KF (11 mg, 0.18 mmol). The mixture was stirred at room temperature for 5 h. After being quenched with water, the mixture was extracted with ethyl acetate. The combined organic layers were washed with brine, dried over anhydrous Na₂SO₄, filtered and concentrated. The residue was purified by column chromatography on silica gel (petroleum ether/ethyl acetate=1/1) to afford 217-1.

Step 2: To a solution of 217-1 (60 mg, 0.080 mmol) in TFA (3 mL) was added TfOH (0.3 mL). The reaction mixture was stirred at room temperature overnight. The mixture was diluted with ethyl acetate and water. The organic layer was washed with saturated aqueous solution of NaHCO₃, brine, dried over anhydrous Na₂SO₄, filtered and concentrated. The residue was purified by prep-HPLC (acetonitrile with 0.05% of TFA in water: 5% to 50%) to afford 217. LCMS (ESI, m/z): [M+H]⁺=629.4; ¹H NMR (400 MHZ, DMSO-d₆, ppm): δ 12.65 (s, 1H), 8.74 (s, 2H), 7.54 (t, J=7.8 Hz, 1H), 7.42-7.38 (m, 2H), 7.33 (d, J=7.6 Hz, 1H), 4.28 (d, J=6.2 Hz, 2H), 3.99-3.80 (m, 6H), 3.75-3.65 (m, 2H), 3.50-3.40 (m, 2H), 3.33-3.27 (m, 2H), 2.02-1.91 (m, 1H), 1.62-1.58 (m, 2H), 1.35-1.25 (m, 2H); ¹⁹F NMR (376 MHz, DMSO-d₆, ppm): δ-58.48 (3F), -59.30 (3F).

### Example 33 Synthesis of Compounds 200 and 201

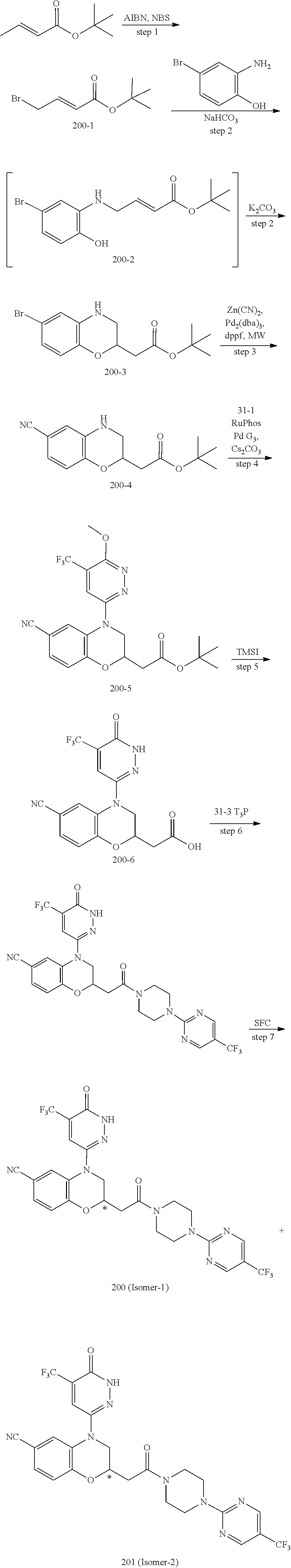

Step 1: To a solution of tert-butyl (E)-but-2-enoate (5.5 g, 38.7 mmol) in CCl₄ (94 mL) were added NBS (5.85 g, 32.9 mmol) and AIBN(318 mg, 1.9 mmol) at 25° C. The mixture was stirred at 80° C. for 16 h under N₂. The mixture was cooled, filtered and the filtrate was concentrated. The residue was purified by column chromatography on silica gel (petroleum to petroleum ether/dichloromethane=3/1) to afford 200-1.

Step 2: To a solution of 2-amino-4-bromophenol (13 g, 69.1 mmol) and 200-1 (20.38 g, 69.1 mmol) in MeOH (200 mL) was added NaHCO₃(7.0 g, 82.97 mmol). The mixture was stirred at 25° C. for 16 h. Then K₂CO₃ (11.5 g, 82.97 mmol) was added. The mixture was stirred at 25° C. for 3 h. The mixture was concentrated. The residue was diluted with ethyl acetate, washed with water, dried over anhydrous Na₂SO₄, filtered and concentrated. The residue was purified by silica gel column chromatography (petroleum ether/ethyl acetate=20/1 to 3/2) to afford 200-3.

Step 3: To a solution of 200-3 (500 mg, 1.52 mmol) and Zn(CN)₂ (358 mg, 3.05 mmol) in DMF (10 mL) were added Pd₂ (dba)₃ (279 mg, 0.305 mmol) and dppf (169 mg, 0.305 mmol). The mixture was stirred at 120° C. for 1.5 h under nitrogen atmosphere and microwave condition. The mixture was cooled, diluted with water, and extracted with ethyl acetate. The combined organic layers were dried over Na₂SO₄, filtered and concentrated. The residue was purified by silica gel column chromatography (dichloromethane/methanol=13/1) to afford 200-4.

Step 4: To a solution of 200-4 (500 mg, 1.82 mmol) and 31-1 (349 mg, 1.64 mmol) in 1,4-dioxane (10 mL) were added RuPhos Pd G³ (305 mg, 0.365 mmol) and Cs₂CO₃ (1.18 g, 3.65 mmol). The mixture was stirred at 120° C. for 1.5 h under nitrogen. The mixture was cooled, diluted with water, and extracted with ethyl acetate. The combined organic layers were dried over Na₂SO₄, filtered and concentrated. The residue was purified by silica gel column chromatography (dichloromethane/methanol=10/1) to afford 200-5.

Step 5: To a solution of 200-5 (500 mg, 1.11 mmol) in acetonitrile (10 mL) was added TMSI (156 mg, 1.11 mmol). The mixture was stirred at 60° C. for 1 h. The mixture was cooled, diluted with ethyl acetate, and washed with saturated aqueous solution of Na₂SO₃ and brine. The organic layer was dried over Na₂SO₄, filtered, and concentrated to afford 200-6 which was used directly in the next step.

Step 6: To a solution of 200-6 (280 mg, 0.74 mmol) and 31-3 (205 mg, 0.88 mmol) in dichloromethane (10 mL) were added DIEA (0.24 mL, 1.47 mmol) and T³P (586 mg, 1.84 mmol). The mixture was stirred at room temperature for 10 min. The mixture was diluted with water, and extracted with ethyl acetate. The combined organic layers were dried over Na₂SO₄, filtered and concentrated. The residue was purified by prep-HPLC (acetonitrile with 0.05% of TFA in water: 5% to 65%) to afford 200-7.

Step 7: Compound 200-7 (150 mg) was purified by SFC (column: REGIS (S,S) WHELK-01, MeOH (+0.1% 7.0 mol/L ammonia in MeOH)/CO₂=60/40) to afford 200 (45 mg) and 201 (52 mg), respectively. 200: SFC analysis: 97.43% ee; retention time: 1.68 min; column: REGIS (S,S) WHELK-01, MeOH (0.1% of DEA) in CO₂, 40%; pressure: 1800 psi; flow rate: 1.5 mL/min. LCMS (ESI, m z): [M+H]=595.4; ¹H NMR (400 MHZ, DMSO-do, ppm): δ 8.74 (s, 2H), 7.98 (s, 1H), 7.63 (s, 1H), 7.31 (d, J=7.6 Hz, 1H), 7.02 (d, J=8.0 Hz, 1H), 4.79-4.67 (m, 1H), 4.02 (d, J=12.4 Hz, 1H), 3.90-3.70 (m, 4H), 3.61-3.55 (m, 4H), 3.60-3.45 (m, 1H), 3.03-2.92 (m, 1H), 2.90-2.75 (m, 1H); ¹⁹F NMR (376 MHz, DMSO-d₆, ppm): δ-59.30 (3F), -65.95 (3F). 201: SFC analysis: 95.66% ee; retention time: 2.09 min; column: REGIS (S,S) WHELK-01, MeOH (0.1% of DEA) in CO₂, 40%; pressure: 1800 psi; flow rate: 1.5 mL/min. LCMS (ESI, m z): [M+H]=595.4; ¹H NMR (400 MHZ, DMSO-d₆, ppm): δ 8.74 (s, 2H), 8.03 (s, 1H), 7.66 (d, J=2.0 Hz, 1H), 7.32 (dd, J=8.4, 2.0 Hz, 1H), 7.03 (d, J=8.4 Hz, 1H), 4.75-4.70 (m, 1H), 4.03 (dd, J=13.2, 2.4 Hz, 1H), 3.91-3.78 (m, 4H), 3.61-3.55 (m, 4H), 3.50 (dd, J=13.2, 7.6 Hz, 1H), 3.02-2.92 (m, 1H), 2.88-2.77 (m, 1H); ¹⁹F NMR (376 MHz, DMSO-d₆, ppm): δ-59.29 (3F), -65.98 (3F).

### Example 34 Synthesis of Compounds 227 and 228

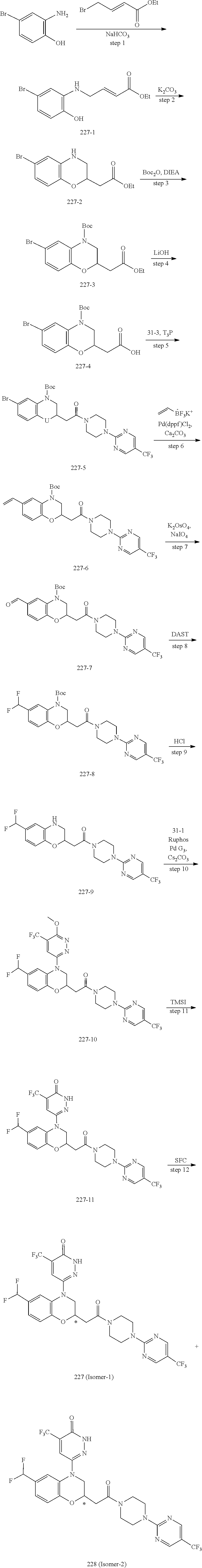

Step 1: To a solution of 2-amino-4-bromophenol (50 g, 265.9 mmol) in MeOH (600 mL) were added ethyl (3E)-5-bromopent-3-enoate (55 g, 265.9 mmol) and NaHCO₃(26.8 g, 319.1 mmol) at 0° C. The mixture was stirred at room temperature for 16 h. After being quenched with ice-water, the mixture was concentrated to remove the organic solvent and the aqueous layer was extracted with ethyl acetate. The combined organic layers were dried over anhydrous Na₂SO₄, filtered, and concentrated to afford 227-1 which was used directly in the next step.

Step 2: To a solution of 227-1 (75 g, 0.25 mol) in MeOH (600 mL) was added K₂CO₃ (6.9 g, 50 mmol) and the mixture was stirred at room temperature for 24 h. The mixture was diluted water and concentrated to remove the organic solvent. The aqueous layer was extracted with dichloromethane and the organic layer was washed with brine, dried over anhydrous Na₂SO₄, filtered and concentrated. The residue was purified by silica gel column chromatography (petroleum ether/ethyl acetate=1/1) to afford 227-2.

Step 3: To a solution of 227-2 (10 g, 33.3 mmol) in THF (150 mL) were added di-tert-butyl dicarbonate (7.7 mL, 33.3 mmol), DMAP (100 mg, 0.82 mmol) and DIEA (3.2 mL, 33.3 mmol). The mixture was stirred at 70° C. for 3 h. The mixture was diluted with ethyl acetate and saturated aqueous solution of NaHCO₃. The organic layer was separated and concentrated. The residue was purified by silica gel column chromatography (petroleum ether/ethyl acetate=5/1) to afford 227-3.

Step 4: To a solution of 227-3 (9 g, 22.5 mmol) in THF (130 mL) and H₂O (100 mL) was added LiOH (4.7 g, 112.4 mmol) at −10° C. and the mixture was stirred at room temperature for 2 h. The mixture was diluted with ethyl acetate and water. The aqueous layer was acidified to pH=2 with 2M HCl and extracted with ethyl acetate. The combined organic layers were concentrated. The residue was purified by silica gel column chromatography (ethyl acetate/dichloromethane=1/1) to afford 227-4.

Step 5: To a solution of 227-4 (8 g, 21.5 mmol) in dichloromethane (25 mL) were added 31-3 (5.99 g, 25.8 mmol) and the mixture was stirred at 0° C. for 30 min. Then DIEA (10.7 mL, 64.5 mmol) and 50% w.t T³P in ethyl acetate (13.7 g, 43.0 mmol) were added and the resulting mixture was stirred at room temperature for another 1 h. The mixture was diluted with dichloromethane and washed with brine. The organic layer was dried over anhydrous Na₂SO₄, filtered and concentrated. The residue was purified by silica gel column chromatography (ethyl acetate/dichloromethane=1/1) to afford 227-5.

Step 6: To a solution of 227-5 (5.7 g, 9.72 mmol) in 1,4-dioxane (100 mL) and H₂O (15 mL) were added potassium trifluoro (vinyl) borate (6.5 g, 48.60 mmol), Cs₂CO₃ (6.3 g, 19.44 mmol) and Pd (dppf) Cl₂ (0.7 g, 0.97 mmol). The reaction mixture was degassed for 10 min. and stirred at 100° C. for 2 h. The mixture was diluted with dichloromethane and brine. The organic layer was separated and concentrated. The residue was purified by silica gel column chromatography eluting with (petroleum ether/dichloromethane/ethyl acetate=1/I/0.5) to afford 227-6.

Step 7: To a solution of 227-6 (4.8 g, 9.0 mmol) in THF (50 mL) and H₂O (25 mL) were added NaIO₄ (7.6 g, 36 mmol) and KOSO₄·2H₂O (166 mg, 0.45 mmol) at 0° C. and the mixture was stirred at room temperature for 2 h. The mixture was diluted with ethyl acetate and saturated aqueous solution of NaHCO₃. The organic layer was separated and concentrated. The residue was purified by silica gel column chromatography (petroleum ether/dichloromethane/ethyl acetate=1/I/0.5) to afford 227-7.

Step 8: To a solution of 227-7 (0.30 g, 0.56 mmol) in dichloromethane (6 mL) was added DAST (1 mL) at −10° C. and the reaction was stirred at room temperature for 18 h. The reaction was diluted with dichloromethane and ice-water. The organic layer was separated, washed with brine, and concentrated. The residue was purified by silica gel column chromatography (petroleum ether/dichloromethane/ethyl acetate=1/I/0.3) to afford 227-8.

Step 9: To a flask containing 227-8 (280 mg, 0.50 mmol) was added 2M HCl in 1,4-dioxane (5 mL). The mixture was stirred at room temperature for 1 h. The mixture was concentrated and purified by silica gel column chromatography (dichloromethane/ethyl acetate=4/1) to afford 227-9.

Step 10: To a solution of 227-9 (168 mg, 0.37 mmol) in 1,4-dioxane (5 mL) were added 31-1 (117 mg, 0.55 mmol), RuPhos Pd G³ (31 mg, 0.037 mmol) and Cs₂CO₃ (240 mg, 0.74 mmol). The reaction mixture was degassed for 10 min. and stirred at 100° C. for 2 h. The mixture was diluted with ethyl acetate and saturated aqueous solution of NaHCO₃. The organic layer was separated and concentrated. The residue was purified by silica gel column chromatography eluting with (dichloromethane/ethyl acetate=1/1) to afford 227-10.

Step 11: To a solution of 227-10 (195 mg, 0.31 mmol) in acetonitrile (5 mL) was added TMSI (86 mg, 0.62 mmol) and the mixture was stirred at 70° C. for 20 min. The mixture was cooled, diluted with ethyl acetate and ice-water. The organic layer was separated and concentrated. The residue was purified by silica gel column chromatography (petroleum ether/dichloromethane/ethyl acetate=1/I/0.5) to afford 227-11.

Step 12:227-11 (179 mg) was purified by SFC (column: DAICELCHIRALPAK®IG, MeOH (+0.1% 7.0 mol/l Ammonia in MeOH)/Supercritical CO₂=30/70) to afford 227 (44.32 mg) and 228 (47.82 mg) respectively. 227: SFC analysis: 99.66% ee; retention time: 3.88 min; column: DAICELCHIRALPAK®IB, MeOH (+0.1% of DEA) in CO₂, 5% to 40%; pressure: 1800 psi; flow rate: 1.5 mL/min. LCMS (ESI, m z): [M+H]=620.4; ¹H NMR (400 MHZ, DMSO-d₆, ppm): 13.19 (s, 1H), 8.77 (s, 2H), 8.05 (s, 1H), 7.35 (s, 1H), 7.15-7.13 (m, 1H), 7.03-7.01 (m, 1H), 6.89 (t, J=56 Hz, 1H), 4.74-4.70 (m, 1H), 4.10-4.05 (m, 1H), 3.96-3.79 (m, 4H), 3.62-3.53 (m, 5H), 3.01-2.95 (m, 1H), 2.86-2.80 (m, 1H). 228: SFC analysis: 97.14% ee; retention time: 4.05 min; column: DAICELCHIRALPAK®IB, MeOH (+0.1% of DEA) in CO₂, 5% to 40%; pressure: 1800 psi; flow rate: 1.5 mL/min. LCMS (ESI, m z): [M+H]=620.4; ¹H NMR (400 MHZ, DMSO-d₆, ppm): 13.22 (s, 1H), 8.80 (s, 2H), 8.08 (s, 1H), 7.37 (s, 1H), 7.17-7.15 (m, 1H), 7.07-7.04 (m, 1H), 6.92 (t, J=56 Hz, 1H), 4.76-4.72 (m, 1H), 4.13-4.08 (m, 1H), 3.98-3.83 (m, 4H), 3.67-3.53 (m, 5H), 3.05-2.98 (m, 1H), 2.89-2.83 (m, 1H).

### Example 35 Synthesis of Compounds 139

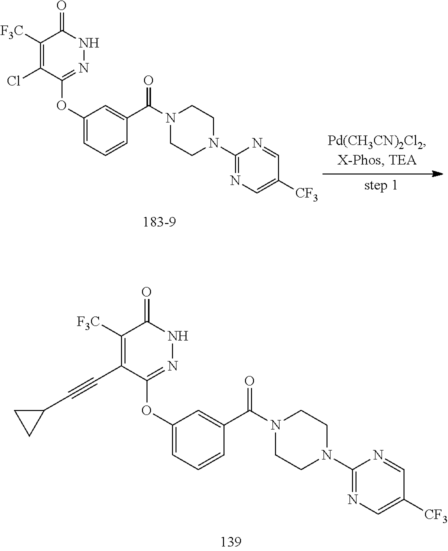 [SMILES: O=C(c1cccc(Oc2n[nH]c(=O)c(C(F)(F)F)c2Cl)c1)N1CCN(c2ncc(C(F)(F)F)cn2)CC1 ; O=C(c1cccc(Oc2n[nH]c(=O)c(C(F)(F)F)c2C#CC2CC2)c1)N1CCN(c2ncc(C(F)(F)F)cn2)CC1]

Step 1: To a solution of 183-9 (50 mg, 0.091 mmol) in DMF (2.5 mL) were added Pd(CH₃CN)₂Cl₂ (47 mg, 0.18 mmol), X-Phos (87 mg, 0.18 mmol), ethynylcyclopropane (120 mg, 1.82 mmol) and TEA (92 mg, 0.91 mmol). The reaction mixture was degassed by bubbling nitrogen for 5 min and then stirred at room temperature for 4 h. After being quenched with H₂O, the mixture was extracted with ethyl acetate and the combined organic layers were washed with water, dried over Na₂SO₄, filtered and concentrated. The residue was purified by prep-HPLC (acetonitrile with 0.05% of TFA in water: 10% to 67%) to afford 139 as 0.19 eq of TFA salt. LCMS (ESI, m/z): [M+H]⁺=579.4; ¹H NMR (400 MHz, DMSO-d₆, ppm): δ 12.92 (s, 1H), 8.75 (s, 2H), 7.55-7.50 (m, 1H), 7.34-7.28 (m, 3H), 3.98-3.82 (m, 4H), 3.79-3.65 (m, 2H), 3.45-3.39 (m, 2H), 1.72-1.65 (m, 1H), 1.06-1.04 (m, 2H), 0.72-0.68 (m, 2H). ¹⁹F NMR (376 MHz, DMSO-d₆, ppm): δ-59.30 (3F), -61.13 (3F).

### Example 36 Synthesis of Compounds 347

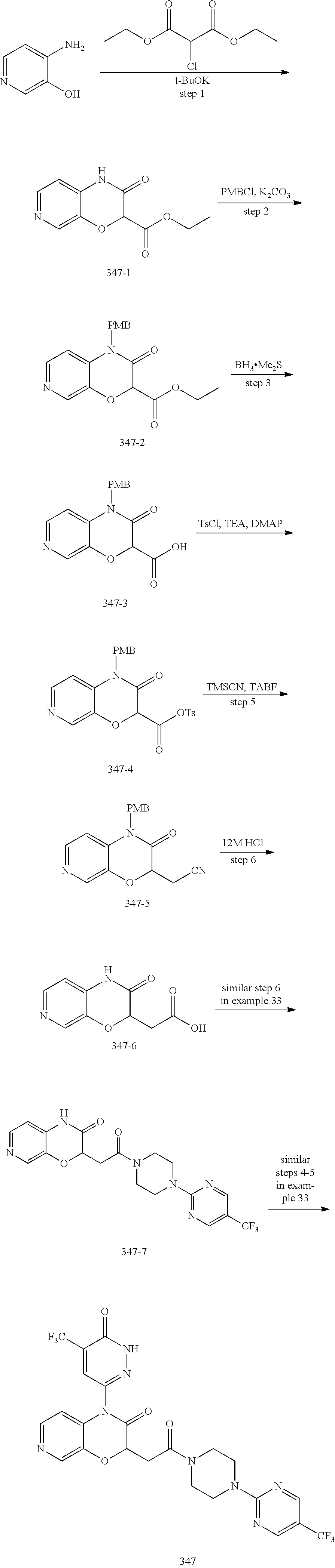

Step 1: To a solution of 4-aminopyridin-3-ol (20 g, 181.6 mmol) in EtOH (300 mL) were added 1,3-diethyl 2-chloropropanedioate (45.95 g, 236.1 mmol) and t-BuOK (40.8 g, 363.2 mmol). Then the reaction mixture was stirred at 80° C. for 2 h. After being cooled to room temperature, the reaction mixture was quenched with H₂O extracted with ethyl acetate. The combined organic layers were dried over Na₂SO₄, filtered and concentrated. The residue was purified by silica gel column chromatography (petroleum ether to petroleum ether/ethyl acetate=1/1) to afford 347-1.

Step 2: To a mixture of 347-1 (12 g, 54.0 mmol) in anhydrous DMF (150 mL) was added in portions K₂CO₃ (22.4 g, 162.0 mmol) at 0° C. The mixture was stirred at this temperature for 5 min. Then 1-(chloromethyl)-4-methoxybenzene (8.6 g, 55.086 mmol) was added dropwise and the resulting mixture was stirred at room temperature for 16 h. After being quenched with H₂O, the mixture was extracted with ethyl acetate. The combined organic layers were washed with brine, dried over anhydrous Na₂SO₄, filtered and concentrated. The residue was purified by silica gel column chromatography (petroleum ether to petroleum ether/ethyl acetate=4/1) to afford 347-2.

Step 3: To a solution of 347-2 (8 g, 23.37 mmol) in dry THF (30 mL) was slowly added BH₃·Me₂S (2M in THF, 19.7 mL) at 0° C. The mixture stirred at 70° C. for 16 h. The reaction mixture was cooled and quenched with methanol. The mixture was stirred at 70° C. for 2 h, then cooled to room temperature. The solvent was removed under reduced pressure to give a residue, which was dissolved in dichloromethane and washed with saturated aqueous NaHCO₃ solution. The organic layer was dried over anhydrous Na₂SO₄, filtered and concentrated to afford 347-3 which was used in the next step directly without further purification.

Step 4: At 25° C., to a mixture of 347-3 (3.0 g, 10.48 mmol) in anhydrous DCM (30 mL) were added TsCl (2.2 g, 11.53 mmol) and DMAP (128.0 mg, 1.05 mmol) and TEA (1.59, 15.72 mmol). The mixture was stirred at this temperature for 5 h. After being quenched with sat. aq. NaHCO₃ solution, the mixture was extracted with ethyl acetate. The combined organic layers were washed with brine, dried over anhydrous Na₂SO₄, filtered and concentrated. The residue was purified by silica gel column chromatography (petroleum ether to petroleum ether/ethyl acetate=4/1) to afford 347-4.

Step 5: To a solution of 347-4 (1.2 g, 2.72 mmol) in THF (40 mL) were added TMSCN(0.95 mL, 8.17 mmol) and TBAF (1 M in THF, 4.1 mL, 4.1 mmol). Then the reaction mixture was stirred at room temperature for 15 h. After being quenched with sat. aq. NaHCO₃ solution, the mixture was extracted with ethyl acetate. The combined organic layers were washed with brine, dried over anhydrous Na₂SO₄, filtered and concentrated. The residue was purified by silica gel column chromatography (petroleum ether to petroleum ether/ethyl acetate=4/1) to afford 347-5.

Step 6: A solution of 347-5 (500 mg, 1.69 mmol) in 12 M HCl (1.41 mL, 16.93 mmol) was stirred at 80° C. for 1.5 h. The mixture was concentrated to afford 347-6 which was used directly in the next step without further purification.

Compound 347-7 was prepared from compound 347-6 and 31-3 following the procedure for the synthesis of compound 200-7 in example 33.

Compound 347 was prepared from compound 347-7 following the procedure for the synthesis of compound 200-6 in example 33 as 3.1 eq of TFA salt. LCMS (ESI, m/z): [M+H]⁺=571.4; ¹H NMR (400 MHZ, methanol-d₄, ppm): δ 8.61 (s, 2H), 8.26 (s, 1H), 8.13 (s, 1H), 8.07 (d, J=6.8 Hz, 1H), 7.38 (d, J=6.8 Hz, 1H), 4.90-4.80 (m, 1H), 4.25-4.15 (m, 1H), 4.05-3.90 (m, 4H), 3.90-3.80 (m, 1H), 3.77-3.65 (m, 4H), 3.20-3.10 (m, 1H), 3.00-2.90 (m, 1H). ¹⁹F NMR (376 MHz, methanol-d₄, ppm): δ-62.64 (3F), -68.97 (3F).

### Example 37 Synthesis of Compound 272

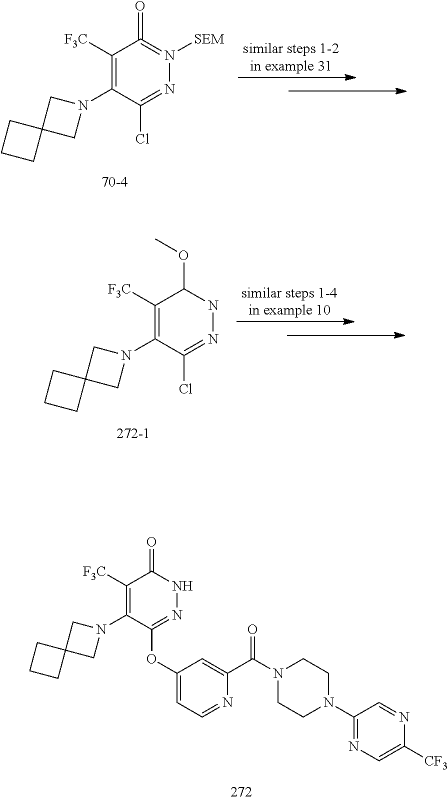

Compound 272-1 was prepared from compound 70-4 following the procedure for the synthesis of compound 236-4 in example 31.

Compound 272 was prepared as a 0.66 eq of TFA salt from compound 272-1 following the procedure for the synthesis of compound 20 in example 10. LCMS (ESI, m/z): [M+H]⁺=611.5; ¹H NMR (400 MHZ, DMSO-d₆, ppm): δ 11.83 (s, 1H), 8.62 (d, J=5.6 Hz, 1H), 8.52 (s, 1H), 8.45 (s, 1H), 7.56 (s, 1H), 7.43 (d, J=5.6 Hz, 1H), 4.37 (s, 4H), 3.92-3.82 (m, 2H), 3.81-3.70 (m, 4H), 3.68-3.58 (m, 2H), 2.20-2.10 (m, 4H), 1.78-1.68 (m, 2H). 19F NMR (376 MHz, DMSO, ppm): δ-51.37 (3H), -64.67 (3H).

Compounds of the present disclosure can be synthesized by those having ordinary skill in the art in view of the present disclosure. Representative further compounds synthesized by following similar procedures/methods described herein in the Examples section. The structures and representative analytical data are shown in Table 1 below.

TABLE 1
Characterization of Representative Compounds

| Com- | | | |
|---|---|---|---|
| pound | | [M + | |
| No. | Structure | H]⁺ | ¹H-NMR and ¹⁹F-NMR |
| 16 | 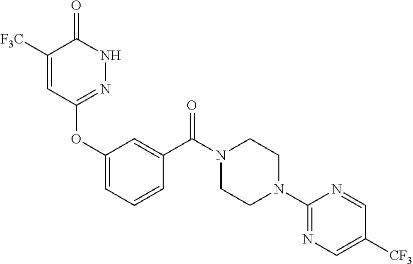 [SMILES: O=C(c1cccc(Oc2cc(C(F)(F)F)c(=O)[nH]n2)c1)N1CCN(c2ncc(C(F)(F)F)cn2)CC1] | 515.2 | ¹H NMR (400 MHz, CDCl₃, ppm) δ 11.51 (s, 1H), 8.50 (s, 2H), 7.57 (s, 1H), 7.49 (t, J = 7.9 Hz, 1H), 7.34 (d, J = 7.7 Hz, 1H), 7.29-7.27 (m, 1H), 7.24-7.22 (m, 1H), 4.10-3.70 (m, 6H), 3.65-3.45 (m, 2H). |
| 18 | 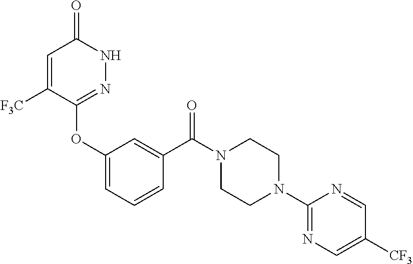 [SMILES: O=C(c1cccc(Oc2n[nH]c(=O)cc2C(F)(F)F)c1)N1CCN(c2ncc(C(F)(F)F)cn2)CC1] | 515.2 | ¹H NMR (400 MHz, CDCl₃, ppm) δ 11.65 (brs, 1H), 8.50 (s, 2H), 7.48 (t, J = 7.9 Hz, 1H), 7.37-7.30 (m, 3H), 7.29- 7.27 (m, 1H), 4.10-3.72 (m, 6H), 3.65-3.45 (m, 2H). |
| 21 | 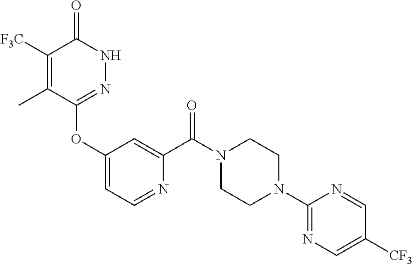 [SMILES: Cc1c(Oc2ccnc(C(=O)N3CCN(c4ncc(C(F)(F)F)cn4)CC3)c2)n[nH]c(=O)c1C(F)(F)F] | 530.3 | ¹H NMR (400 MHz, CDCl₃, ppm) δ 11.10 (s, 1H), 8.62- 8.51 (m, 3H), 7.57 (s, 1H), 7.26-7.20 (m, 1H), 4.06-3.82 (m, 8H), 2.49 (s, 3H). |
| 22 | 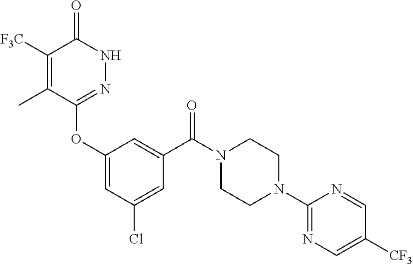 [SMILES: Cc1c(Oc2cc(Cl)cc(C(=O)N3CCN(c4ncc(C(F)(F)F)cn4)CC3)c2)n[nH]c(=O)c1C(F)(F)F] | 563.2 | ¹HNMR (400 MHz, CDCl₃, ppm): δ 8.52 (s, 2H), 7.32 (s, 1H), 7.25 (s, 1H), 7.16 (s, 1H), 4.10-3.48 (m, 8H), 2.52 (m, 3H). ¹⁹FNMR (376 MHz, DMSO-d₆, ppm): δ −58.50 (3F), −59.34 (3F). |
| 23 | 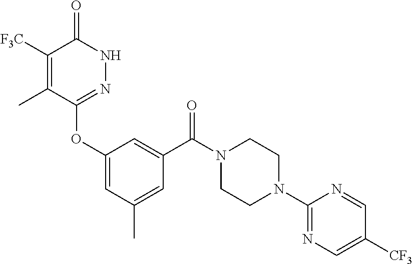 [SMILES: Cc1cc(Oc2n[nH]c(=O)c(C(F)(F)F)c2C)cc(C(=O)N2CCN(c3ncc(C(F)(F)F)cn3)CC2)c1] | 543.3 | ¹H NMR (400 MHz, DMSO-d₆, ppm): δ 12.71 (s, 1H), 8.72 (s, 2H), 7.16-7.08 (m, 3H), 3.95-3.88 (m, 4H), 3.70-3.63 (m, 4H), 2.38 (s, 3H), 2.34 (s, 3H). |
| 24 | 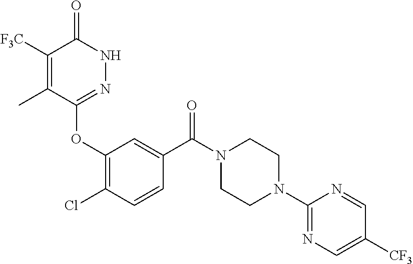 [SMILES: Cc1c(Oc2cc(C(=O)N3CCN(c4ncc(C(F)(F)F)cn4)CC3)ccc2Cl)n[nH]c(=O)c1C(F)(F)F] | 563.2 | ¹H-NMR (400 MHz, DMSO-d₆, ppm) δ 12.76 (s, 1H), 8.72 (s, 2H), 7.69 (d, J = 8.4 Hz, 1H), 7.56 (d, J = 1.6 Hz, 1H), 7.37 (dd, J = 8.0, 2.0 Hz, 1H), 3.94-3.89 (m, 4H), 3.72-3.63 (m, 2H), 3.50-3.35 (m, 2H), 2.56- 2.33 (m, 3H). |
| 25 | 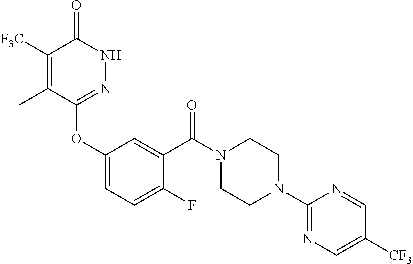 [SMILES: Cc1c(Oc2ccc(F)c(C(=O)N3CCN(c4ncc(C(F)(F)F)cn4)CC3)c2)n[nH]c(=O)c1C(F)(F)F] | 547.2 | ¹HNMR (400 MHz, CD₃OD, ppm): δ 8.59 (s, 2H), 7.37- 7.27 (m, 3H), 4.06-4.04 (m, 2H), 3.96-3.94 (m, 2H), 3.87- 3.84 (m, 2H), 3.50-3.47 (m, 2H), 2.52-2.50 (q, J = 2.8 Hz, 3H). ¹⁹FNMR (376 MHz, CD₃OD, ppm): δ −60.70 (3F), −62.70 (3F), −121.94 (1F). |
| 26 | 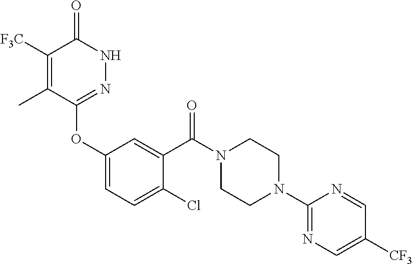 [SMILES: Cc1c(Oc2ccc(Cl)c(C(=O)N3CCN(c4ncc(C(F)(F)F)cn4)CC3)c2)n[nH]c(=O)c1C(F)(F)F] | 562.8 | ¹H NMR (400 MHz, Methanol- d₄) δ 8.59 (s, 2H), 7.56 (dt, J = 9.1, 1.4 Hz, 1H), 7.34-.29 (m, 2H), 4.06 (t, J = 5.3 Hz, 2H), 3.97-3.91 (m, 2H), 3.89- 3.82 (m, 2H), 3.45-3.40 (m, 2H), 2.50 (q, J = 2.8 Hz, 3H). |
| 29 | 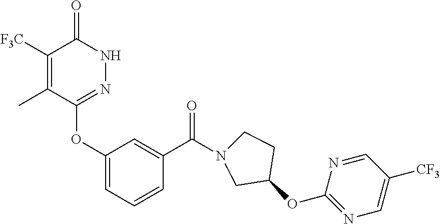 [SMILES: Cc1c(Oc2cccc(C(=O)N3CC[C@@H](Oc4ncc(C(F)(F)F)cn4)C3)c2)n[nH]c(=O)c1C(F)(F)F] | 530.2 | ¹H NMR (400 MHz, Methanol- d₄) δ 8.91 (d, J = 23.2 Hz, 2H), 7.57-7.21 (m, 4H), 5.90- 5.55 (m, 1H), 4.12-3.57 (m, 4H), 2.51 (dd, J = 5.6, 2.8 Hz, 3H), 2.43-2.23 (m, 2H). |
| 30 | 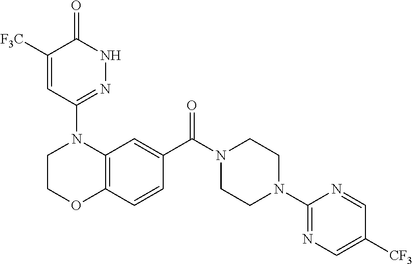 [SMILES: O=C(c1ccc2c(c1)N(c1cc(C(F)(F)F)c(=O)[nH]n1)CCO2)N1CCN(c2ncc(C(F)(F)F)cn2)CC1] | 556.2 | ¹H NMR (400 MHz, methanol-d₄, ppm): δ 8.59 (s, 2H), 8.03 (s, 1H), 7.17 (d, J = 1.9 Hz, 1H), 7.07 (dd, J = 8.3, 1.9 Hz, 1H), 6.99 (d, J = 8.3 Hz, 1H), 4.45-4.30 (m, 2H), 4.02-3.92 (m, 4H), 3.87-3.79 (m, 2H), 3.80-3.64 (m, 4H). |
| 33 | 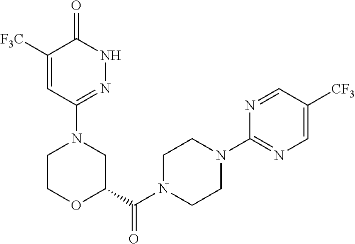 [SMILES: O=C([C@H]1CN(c2cc(C(F)(F)F)c(=O)[nH]n2)CCO1)N1CCN(c2ncc(C(F)(F)F)cn2)CC1] | 508.2 | 0.7TFA salt, ¹H NMR (400 MHz, DMSO-d₆, ppm): δ 12.79 (s, 1H), 8.72 (s, 2H), 7.98 (s, 1H), 4.41-4.38 (m, 1H), 3.91- 3.79 (m, 6H), 3.70-3.64 (m, 4H), 3.57-3.55 (m, 2H), 3.02- 2.97 (m, 1H), 2.92-2.85 (m, 1H). ¹⁹F NMR (376 MHz, DMSO-d₆, ppm): δ −59.32 (3F), −65.74 (3F). |
| 37 | 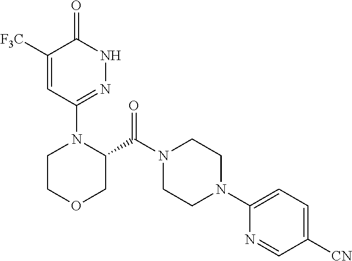 [SMILES: N#Cc1ccc(N2CCN(C(=O)[C@@H]3COCCN3c3cc(C(F)(F)F)c(=O)[nH]n3)CC2)nc1] | 486.2 [M + Na]⁺ | ¹H NMR (400 MHz, methanol-d₄, ppm): δ 8.43 (d, J = 2.0 Hz, 1H), 7.88 (s, 1H), 7.78-7.75 (dd, J = 8.8, 2.4 Hz, 1H), 6.88 (d, J = 9.2 Hz, 1H), 4.54 (s, 1H), 4.04-3.47 (m, 14H). ¹⁹F NMR (376 MHz, methanol-d₄, ppm): δ −68.57 (3F). |
| 38 | 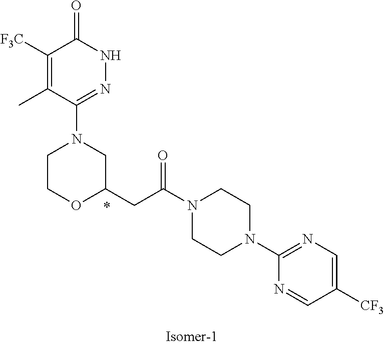 [SMILES: Cc1c(N2CCOC(CC(=O)N3CCN(c4ncc(C(F)(F)F)cn4)CC3)C2)n[nH]c(=O)c1C(F)(F)F] | 536.3 | SFC analysis: 99.10% ee; retention time: 1.132 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 4.14-4.08 (m, 1H), 4.05-3.86 (m, 5H), 3.85-3.80 (m, 1H), 3.77-3.60 (m, 4H), 3.28-3.24 (m, 1H), 3.09-3.06 (m, 1H), 2.90-2.77 (m, 2H), 2.68-2.62 (m, 1H), 2.57-2.52 (m, 1H), 2.48-2.45 (m, 3H). |
| 39 | 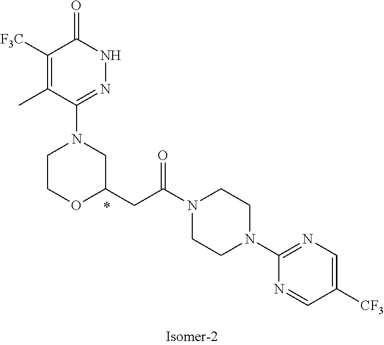 [SMILES: Cc1c(N2CCOC(CC(=O)N3CCN(c4ncc(C(F)(F)F)cn4)CC3)C2)n[nH]c(=O)c1C(F)(F)F] | 536.3 | SFC analysis: 90.74% ee; retention time: 1.320 min; column: Regis Whelk-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 4.14-4.08 (m, 1H), 4.06-3.86 (m, 5H), 3.85-3.79 (m, 1H), 3.78-3.60 (m, 4H), 3.28-3.24 (m, 1H), 3.10-3.06 (m, 1H), 2.90-2.77 (m, 2H), 2.68-2.62 (m, 1H), 2.57-2.52 (m, 1H), 2.48-2.45 (m, 3H). |
| 40 | 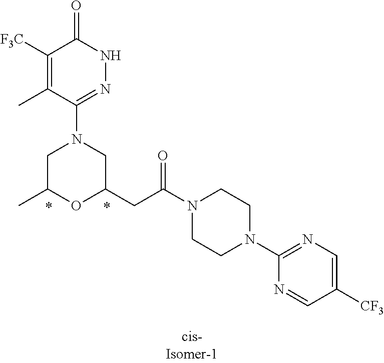 [SMILES: Cc1c(N2CC(C)OC(CC(=O)N3CCN(c4ncc(C(F)(F)F)cn4)CC3)C2)n[nH]c(=O)c1C(F)(F)F] | 550.0 | SFC analysis: 99.52% ee; retention time: 2.176 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 4.55 (s, 2H), 4.18- 4.14 (m, 1H), 4.07-3.98 (m, 2H), 3.94-3.72 (m, 4H), 3.69- 3.58 (m, 2H), 3.31-3.25 (m, 1H), 3.14-3.09 (m, 1H), 2.84- 2.78 (m, 1H), 2.61-2.50 (m, 2H), 2.49-2.45 (m, 3H), 1.17 (d, J = 6.2 Hz, 3H). |
| 41 | 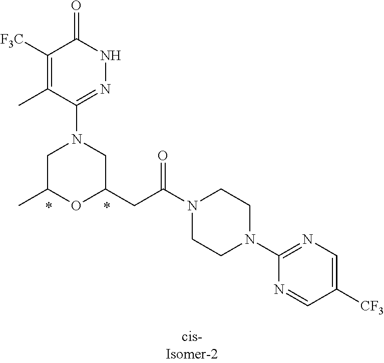 [SMILES: Cc1c(N2CC(C)OC(CC(=O)N3CCN(c4ncc(C(F)(F)F)cn4)CC3)C2)n[nH]c(=O)c1C(F)(F)F] | 550.0 | SFC analysis: 97.52% ee; retention time: 2.410 min; column: DAICELCHIRALPAK ® OJ, 100 × 3.0 mm,3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.60 (s, 2H), 4.55 (s, 2H), 4.19- 4.13 (m, 1H), 4.07-3.98 (m, 2H), 3.95-3.82 (m, 2H), 3.80- 3.72 (m, 2H), 3.70-3.60 (m, 2H), 3.30-3.25 (m, 1H), 3.14- 3.12 (m, 1H), 2.84-2.78 (m, 1H), 2.60-2.54 (m, 2H), 2.48- 2.45 (m, 3H), 1.17 (d, J = 8 Hz, 3H). |
| 46 | 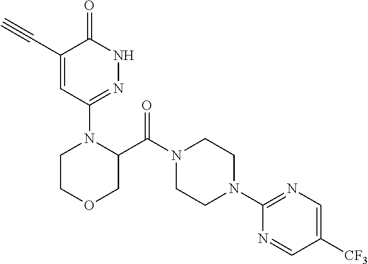 [SMILES: C#Cc1cc(N2CCOCC2C(=O)N2CCN(c3ncc(C(F)(F)F)cn3)CC2)n[nH]c1=O] | 464.1 | ¹H NMR (400 MHz, methanol-d₄, ppm): δ 8.60 (s, 2H), 7.72 (s, 1H), 4.79 (s, 1H), 4.26-4.05 (d, J = 18.6, Hz, 3H), 4.04- 3.89 (m, 4H), 3.88-3.68 (m, 6H), 3.51-3.37 (m, 2H). |
| 47 | 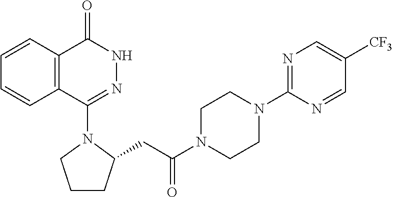 [SMILES: O=C(C[C@@H]1CCCN1c1n[nH]c(=O)c2ccccc12)N1CCN(c2ncc(C(F)(F)F)cn2)CC1] | 487.9 | ¹H NMR (400 MHz, methanol-d₄, ppm): δ 8.59 (s, 2H), 8.34 (dd, J = 8.0, 1.2 Hz, 1H), 8.13 (d, J = 8.4 Hz, 1H), 7.92-7.90 (m, 1H), 7.86-7.84 (m, 1H), 4.47- 4.43 (m, 1H), 4.02-3.98 (m, 3H), 3.83-3.50 (m, 6H), 3.32 (m, 1H), 3.12-3.07 (m, 1H), 2.40-2.39 (m, 1H), 2.37-2.35 (m, 1H), 2.07-2.04 (m, 1H), 1.87-1.82 (m, 2H). ¹⁹F NMR (376 MHz, methanol-d₄, ppm): δ −62.64 (3F). |
| 51 | 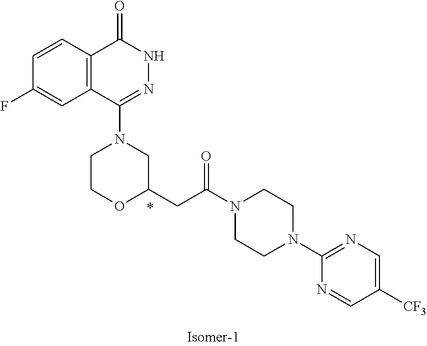 [SMILES: O=C(CC1CN(c2n[nH]c(=O)c3ccc(F)cc23)CCO1)N1CCN(c2ncc(C(F)(F)F)cn2)CC1] | 522.2 | SFC analysis: 99.58% ee; retention time: 3.255 min; column: DAICELCHIRALPAK ® OJ, 100 × 3.0 mm, 3 μm, A for CO₂ and B for MeOH (0.1% DEA), 5% to 40%; pressure: 100 bar, flow rate: 1.5 mL/min. ¹H NMR (400 MHz, methanol-d₄, ppm): δ 8.59 (s, 2H), 8.40 (dd, J = 8.8, 5.5 Hz, 1H), 7.71 (dd, J = 9.4, 2.5 Hz, 1H), 7.61 (td, J = 8.6, 2.5 Hz, 1H), 4.32-4.21 (m, 1H), 4.07-3.84 (m, 6H), 3.81- 3.61 (m, 4H), 3.53-3.45 (m, 1H), 3.35-3.32 (m, 1H), 3.04- 2.97 (m, 1H), 2.87-2.76 (m, 2H), 2.64-2.55 (m, 1H). |
| 52 | 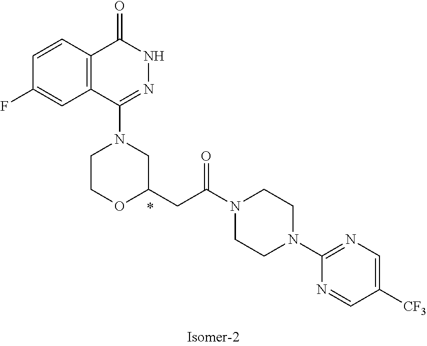 [SMILES: O=C(CC1CN(c2n[nH]c(=O)c3ccc(F)cc23)CCO1)N1CCN(c2ncc(C(F)(F)F)cn2)CC1] | 522.2 | SFC analysis: 98.28% ee; retention time: 3.455 min; column: DAICEL CHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.40 (dd, J = 8.8, 5.5 Hz, 1H), 7.71 (dd, J = 9.4, 2.4 Hz, 1H), 7.61 (td, J = 8.6, 2.5 Hz, 1H), 4.32-4.18 (m, 1H), 4.10-3.83 (m, 6H), 3.81- 3.59 (m, 4H), 3.53-3.46 (m, 1H), 3.35-3.32 (m, 1H), 3.06- 2.95 (m, 1H), 2.89-2.75 (m, 2H), 2.65-2.56 (m, 1H). |
| 53 | 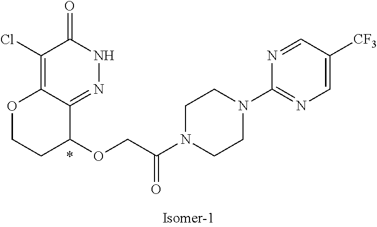 [SMILES: O=C(COC1CCOc2c1n[nH]c(=O)c2Cl)N1CCN(c2ncc(C(F)(F)F)cn2)CC1] | 475.4 | SFC analysis: 100% ee; retention time; 1.428 min; column: DAICEL CHIRALPAK ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, DMSO-d₆): δ 13.27 (s, 1H), 8.75 (s, 2H), 4.52-4.45 (m, 2H), 4.41-4.36 (m, 3H), 3.90-3.73 (m, 4H), 3.58-3.40 (m, 4H), 2.34-2.14 (m, 2H). |
| 54 | 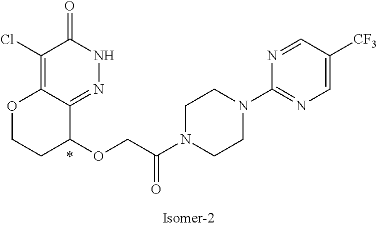 [SMILES: O=C(COC1CCOc2c1n[nH]c(=O)c2Cl)N1CCN(c2ncc(C(F)(F)F)cn2)CC1] | 475.2 | SFC analysis: 97.51% ee; retention time: 2.634 min; column: DAICEL CHIRALPAK ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, DMSO-d₆): δ 13.28 (s, 1H), 8.75 (s, 2H), 4.52-4.47 (m, 2H), 4.44-4.36 (m, 3H), 3.90-3.73 (m, 4H), 3.58-3.40 (m, 4H), 2.34-2.15 (m, 2H). |
| 55 | 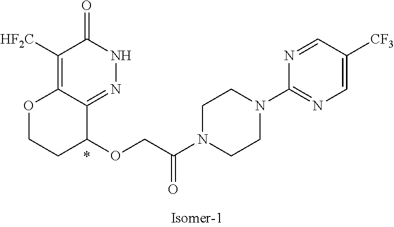 | 491.4 | SFC analysis: 100% ee; retention time: 7.331 min; column: DAICEL CHIRALPAK ® AY, 250 × 4.6 mm, 5 μm, A for n- hexane and B for EtOH (0.2% DEA), 40%; pressure: 100 bar; flow rate: 1.0 mL/min. ¹H NMR (400 MHz, DMSO-d₆): δ 13.16 (s, 1H), 8.72 (s, 2H), 6.89 (t, J = 53.3 Hz, 1H), 4.51- 4.32 (m, 5H), 3.90-3.70 (m, 4H), 3.55- 3.40 (m, 4H), 2.33-2.13 (m, 2H). |
| 56 | 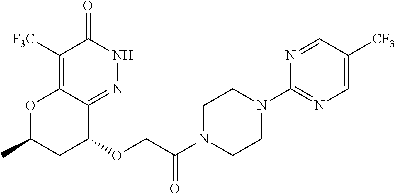 [SMILES: C[C@@H]1C[C@@H](OCC(=O)N2CCN(c3ncc(C(F)(F)F)cn3)CC2)c2n[nH]c(=O)c(C(F)(F)F)c2O1] | 523.4 | ¹H NMR (400 MHz, DMSO-d₆): δ 13.25 (s, 1H), 8.74 (s, 2H), 4.76-4.68 (m, 1H), 4.49-4.45 (m, 1H), 4.37 (s, 2H), 3.88-3.68 (m, 5H), 3.46-3.34 (m, 3H), 2.38-2.33 (m, 1H), 2.02-1.94 (m, 1H), 1.41 (d, J = 6.4 Hz, 3H). ¹⁹F NMR (376 MHz, DMSO-d₄, ppm): δ −57.37 (3F), −59.30 (3F). |
| 57 | 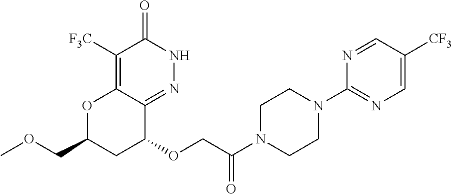 [SMILES: COC[C@@H]1C[C@@H](OCC(=O)N2CCN(c3ncc(C(F)(F)F)cn3)CC2)c2n[nH]c(=O)c(C(F)(F)F)c2O1] | 553.4 | 0.13 TFA salt, ¹H NMR (400 MHz, DMSO-d₆): δ 13.28 (s, 1H), 8.75 (s, 2H), 4.78-4.71 (m, 1H), 4.55-4.51 (m, 1H), 4.39-4.32 (m, 2H), 3.97-3.78 (m, 3H), 3.75-3.60 (m, 3H), 3.58-3.41 (m, 4H), 3.40-3.33 (m, 3H), 2.37-2.24 (m, 1H), 2.15-2.03 (m, 1H); ¹⁹F NMR (376 MHz, DMSO-d₆, ppm): δ −57.41 (3F), −59.30 (3F). |
| 58 | 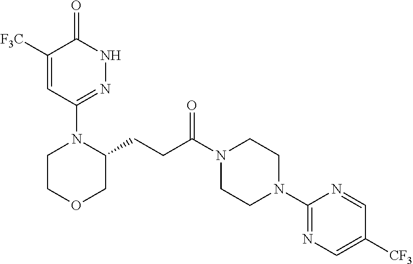 [SMILES: O=C(CC[C@@H]1COCCN1c1cc(C(F)(F)F)c(=O)[nH]n1)N1CCN(c2ncc(C(F)(F)F)cn2)CC1] | 536.2 | ¹H NMR (400 MHz, methanol-d₄, ppm): δ 8.59 (s, 2H), 7.87 (s, 1H), 4.08-3.78 (m, 7H), 3.76-3.50 (m, 7H), 3.30-3.20 (m, 1H), 2.53-2.43 (m, 2H), 2.18-1.96 (m, 2H). |
| 59 | 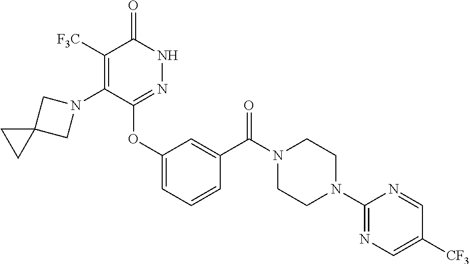 [SMILES: O=C(c1cccc(Oc2n[nH]c(=O)c(C(F)(F)F)c2N2CC3(CC3)C2)c1)N1CCN(c2ncc(C(F)(F)F)cn2)CC1] | 596.4 | 0.9 TFA salt, ¹H NMR (400 MHz, methanol-d₄, ppm): δ 8.60 (s, 2H), 7.58-7.50 (m, 1H), 7.38-7.29 (m, 3H), 4.62 (s, 4H), 4.10-3.89 (m, 4H), 3.89-3.77 (m, 2H), 3.63-3.48 (m, 2H), 0.72 (s, 4H); ¹⁹F NMR (376 MHz, methanol-d₄, ppm) δ −53.37, −62.66. |
| 60 | 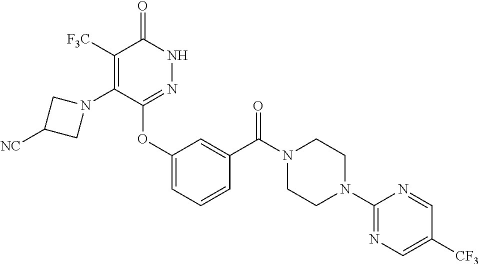 [SMILES: N#CC1CN(c2c(Oc3cccc(C(=O)N4CCN(c5ncc(C(F)(F)F)cn5)CC4)c3)n[nH]c(=O)c2C(F)(F)F)C1] | 595.4 | 2.2 TFA salt, ¹H NMR (400 MHz, methanol-d₄, ppm): δ 8.60 (s, 2H), 7.57-7.52 (m, 1H), 7.38-7.32 (m, 3H), 4.88- 4.82 (m, 2H), 4.77-4.72 (m, 2H), 4.10-3.90 (m, 4H), 3.89- 3.75 (m, 3H), 3.62-3.52 (m, 2H). ¹⁹F NMR (376 MHz, methanol-d₄, ppm) δ −53.73, −62.66. |
| 61 | 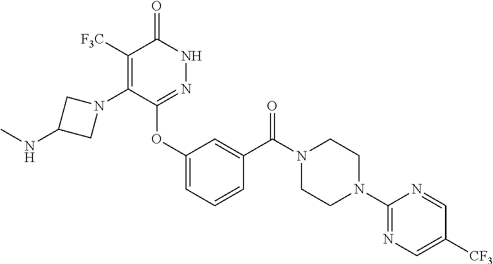 [SMILES: CNC1CN(c2c(Oc3cccc(C(=O)N4CCN(c5ncc(C(F)(F)F)cn5)CC4)c3)n[nH]c(=O)c2C(F)(F)F)C1] | 599.5 | 1.5 TFA salt, ¹H NMR (400 MHz, DMSO-d₆): δ 8.75 (s, 2H), 7.57-7.53 (m, 1H), 7.34-7.32 (m, 3H), 4.70-4.65 (m, 2H), 4.43-4.40 (m, 2H), 3.90-3.73 (m, 9H), 3.51-3.45 (m, 3H). ¹⁹F NMR (376 MHz, DMSO-d₆, ppm): δ −51.53 (3F), −59.29 (3F). |
| 63 | 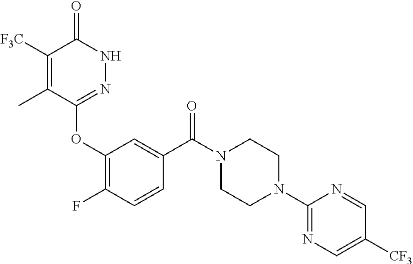 [SMILES: Cc1c(Oc2cc(C(=O)N3CCN(c4ncc(C(F)(F)F)cn4)CC3)ccc2F)n[nH]c(=O)c1C(F)(F)F] | 547.2 | ¹H NMR (400 MHz, methanol-d₆, ppm): δ 8.60 (s, 2H), 7.52 (dd, J = 7.3, 1.9 Hz, 1H), 7.46-7.35 (m, 2H), 4.10-3.50 (m, 8H), 2.55-2.50 (m, 3H). |
| 64 | 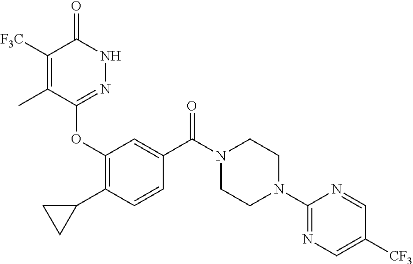 [SMILES: Cc1c(Oc2cc(C(=O)N3CCN(c4ncc(C(F)(F)F)cn4)CC3)ccc2C2CC2)n[nH]c(=O)c1C(F)(F)F] | 569.3 | ¹H NMR (400 MHz, methanol-d₄, ppm): δ 8.59 (s, 2H), 7.33-7.25 (m, 2H), 7.13 (d, J = 7.9 Hz, 1H), 3.98-3.50 (m, 8H), 2.55-2.50 (m, 3H), 2.02-1.91 (m, 1H), 0.99-0.89 (m, 2H), 0.75-0.63 (m, 2H). |
| 65 | 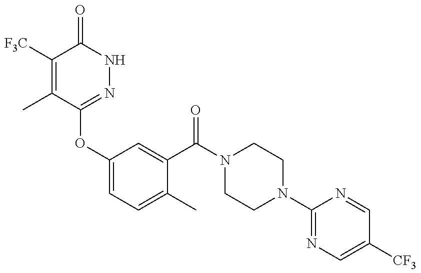 [SMILES: Cc1ccc(Oc2n[nH]c(=O)c(C(F)(F)F)c2C)cc1C(=O)N1CCN(c2ncc(C(F)(F)F)cn2)CC1] | 543.0 | 0.5TFA salt, ¹H NMR (400 MHz, DMSO-d₆, ppm): δ 12.68 (s, 1H), 8.71 (s, 2H), 7.32-7.30 (m, 1H), 7.17-7.12 (m, 2H), 3.95-3.79 (m, 6H), 3.27-3.25 (m, 2H), 2.40-2.35 (m, 3H), 2.21 (s, 3H). ¹⁹F NMR (376 MHz, DMSO-d₆, ppm): δ −58.41 (3F), −59.34 (3F). |
| 66 | 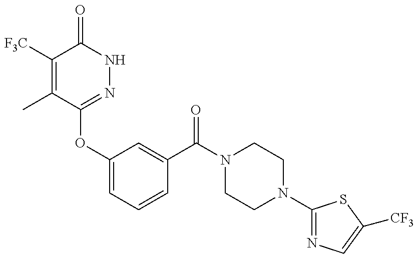 [SMILES: Cc1c(Oc2cccc(C(=O)N3CCN(c4ncc(C(F)(F)F)s4)CC3)c2)n[nH]c(=O)c1C(F)(F)F] | 534.3 | ¹H NMR (400 MHz, methanol-d₄, ppm): δ 7.62-7.48 (m, 2H), 7.41-7.27 (m, 3H), 4.01-3.81 (m, 2H), 3.76-3.52 (m, 6H), 2.55-2.50 (m, 3H). |
| 67 | 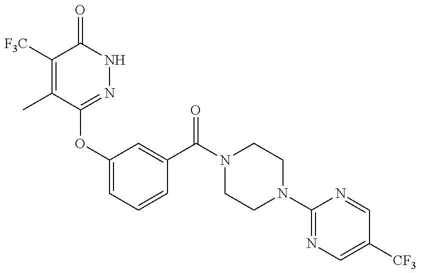 [SMILES: Cc1c(Oc2cccc(C(=O)N3CCN(c4ncc(C(F)(F)F)cn4)CC3)c2)n[nH]c(=O)c1C(F)(F)F] | 529.2 | ¹H NMR (400 MHz, methanol-d₄, ppm): δ 8.54-8.24 (m, 2H), 7.55 (dd, J = 10.6, 5.4 Hz, 1H), 7.45-7.23 (m, 3H), 3.95-3.77 (m, 6H), 3.69-3.54 (m, 2H), 2.54-2.50 (m, 3H). |
| 68 | 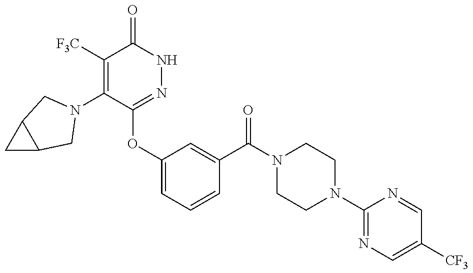 [SMILES: O=C(c1cccc(Oc2n[nH]c(=O)c(C(F)(F)F)c2N2CC3CC3C2)c1)N1CCN(c2ncc(C(F)(F)F)cn2)CC1] | 596.4 | ¹H NMR (400 MHz, DMSO-d₆, ppm): δ 11.92 (s, 1H), 8.75 (s, 2H), 7.54-7.50 (m, 1H), 7.34-7.29 (m, 3H), 3.98-3.85 (m, 6H), 3.80-3.40 (m, 6H), 1.57-1.55 (m, 2H), 0.55-0.50 (m, 1H), 0.30-0.25 (m, 1H). ¹⁹F NMR (376 MHz, DMSO-d₆, ppnm) δ −56.92 (3F), −59.30 (3F). |
| 69 | 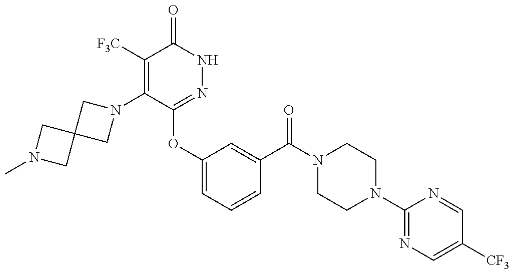 [SMILES: CN1CC2(C1)CN(c1c(Oc3cccc(C(=O)N4CCN(c5ncc(C(F)(F)F)cn5)CC4)c3)n[nH]c(=O)c1C(F)(F)F)C2] | 625.5 | 3.3 TFA salt, ¹H NMR (400 MHz, DMSO-d₆): δ 11.66 (s, 1H), 8.74 (s, 2H), 7.56-7.52 (m, 1H), 7.33-7.20 (m, 3H), 4.60 (s, 4H), 4.30-4.10 (m, 4H), 3.99- 3.80 (m, 4H), 3.78- 3.65 (m, 4H), 2.73 (s, 3H). ¹⁹F NMR (376 MHz, DMSO-d₆, ppm): δ −51.50 (3F), −59.30 (3F). |
| 72 | 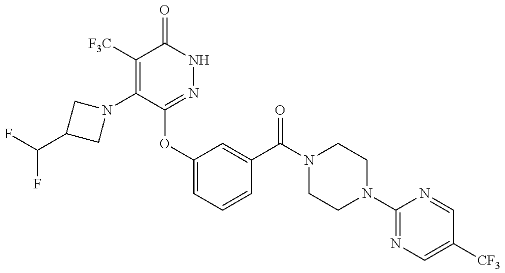 [SMILES: O=C(c1cccc(Oc2n[nH]c(=O)c(C(F)(F)F)c2N2CC(C(F)F)C2)c1)N1CCN(c2ncc(C(F)(F)F)cn2)CC1] | 620.4 | 1.0 TFA salt, ¹H NMR (400 MHz, DMSO-d₆): δ 11.70 (s, 1H), 8.74 (s, 2H), 7.66-7.05 (m, 4H), 6.52-6.23 (m, 1H), 4.57-4.37 (m, 4H), 4.01-3.61 (m, 7H), 3.30-3.20 (m, 2H). ¹⁹F NMR (376 MHz, DMSO-d₆, ppm): δ −51.45 (3F), −59.29 (3F), −124.85 (2F). |
| 74 | 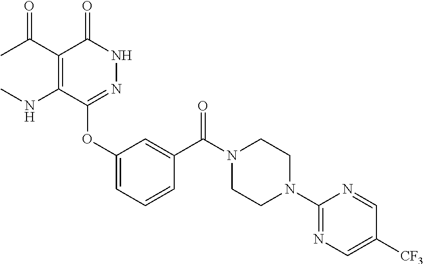 [SMILES: CNc1c(Oc2cccc(C(=O)N3CCN(c4ncc(C(F)(F)F)cn4)CC3)c2)n[nH]c(=O)c1C(C)=O] | 518.1 | ¹H NMR (300 MHz, methanol-d₄, ppm): δ 8.60-8.59 (m, 2H), 7.37 (t, J = 7.8 Hz, 1 H), 7.03-6.98 (m, 2H), 6.85-6.84 (m, 1H), 4.03-3.93 (m, 4H), 3.81- 3.79 (m, 2H), 3.55-3.53 (m, 2H), 3.24 (s, 3H), 2.29 (s, 3H). ¹⁹F NMR (282 MHz, DMSO-d₄, ppm): δ −62.66 (3F). |
| 77 | 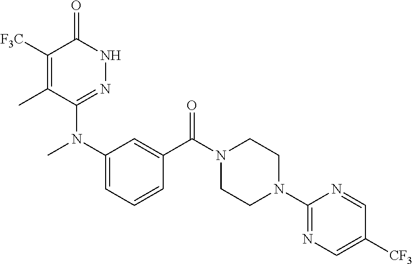 [SMILES: Cc1c(N(C)c2cccc(C(=O)N3CCN(c4ncc(C(F)(F)F)cn4)CC3)c2)n[nH]c(=O)c1C(F)(F)F] | 541.9 | 3.2TFA salt, ¹H NMR (400 MHz, DMSO-d₆, ppm): δ 13.28 (s, 1H), 8.71 (s, 2H), 7.30 (d, J = 7.9 Hz, 1H), 7.00-6.73 (m, 3H), 3.90-3.86 (m, 4H), 3.67-3.31 (m, 4H), 3.19 (s, 3H), 2.07 (s, 3H). ¹⁹F NMR (376 MHz, DMSO-d₆, ppm): δ −57.19 (3F), −59.33 (3F). |
| 78 | 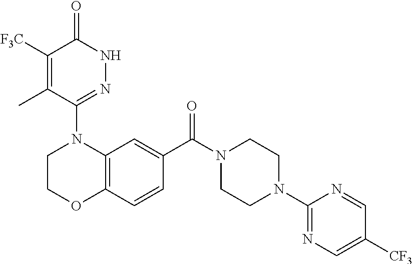 [SMILES: Cc1c(N2CCOc3ccc(C(=O)N4CCN(c5ncc(C(F)(F)F)cn5)CC4)cc32)n[nH]c(=O)c1C(F)(F)F] | 570.3 | ¹H NMR (400 MHz, methanol-d₄, ppm): δ 8.58 (s, 2H), 6.95-6.91 (m, 2H), 6.53-6.49 (m, 1H), 4.45-4.38 (m, 2H), 4.01-3.87 (m, 4H), 3.76-3.52 (m, 6H), 2.43-2.39 (m, 3H). |
| 79 | 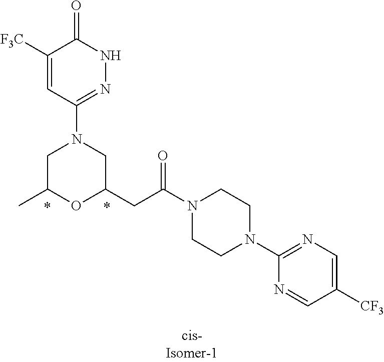 [SMILES: CC1CN(c2cc(C(F)(F)F)c(=O)[nH]n2)CC(CC(=O)N2CCN(c3ncc(C(F)(F)F)cn3)CC2)O1] | 536.0 | SFC analysis: 97.32% ee; retention time: 1.679 min; column: DAICELCHIRALPAK ® OJ, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 7.88 (s, 1H), 4.08- 3.99 (m, 3H), 3.95-3.71 (m, 7H), 3.70-3.59 (m, 2H), 2.86- 2.81 (m, 1H), 2.63-2.45 (m, 3H), 1.19 (d, J = 6.2 Hz, 3H). |
| 82 | 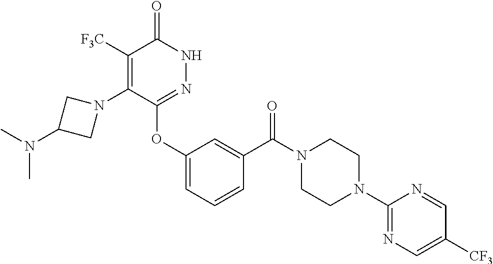 [SMILES: CN(C)C1CN(c2c(Oc3cccc(C(=O)N4CCN(c5ncc(C(F)(F)F)cn5)CC4)c3)n[nH]c(=O)c2C(F)(F)F)C1] | 613.4 | 4.4 TFA salt, ¹H NMR (400 MHz, DMSO-d₆): δ 11.72 (s, 1H), 8.74 (s, 2H), 7.58-7.50 (m, 1H), 7.42-7.30 (m, 3H), 4.71-4.58 (m, 4H), 4.03-3.82 (m, 5H), 3.76-3.63 (m, 4H), 2.68 (s, 6H). ¹⁹F NMR (376 MHz, DMSO-d₆, ppm): δ −51.69 (3F), −59.30 (3F). |
| 83 | 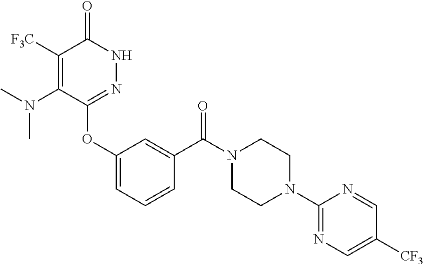 [SMILES: CN(C)c1c(Oc2cccc(C(=O)N3CCN(c4ncc(C(F)(F)F)cn4)CC3)c2)n[nH]c(=O)c1C(F)(F)F] | 558.2 | 2.8 TFA salt, ¹H NMR (400 MHz, DMSO-d₆): δ 12.00 (s, 1H), 8.74 (s, 2H), 7.61-7.20 (m, 4H), 4.06-3.82 (m, 4H), 3.71-3.60 (m, 4H), 3.07 (s, 6H). ¹⁹F NMR (376 MHz, DMSO-d₆, ppm): δ −58.10 (3F), −59.30 (3F). |
| 84 | 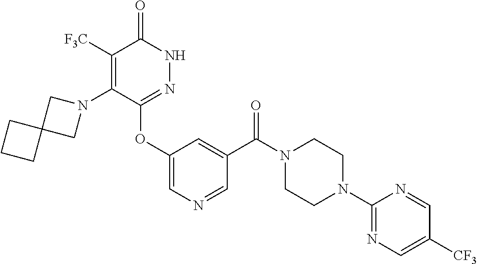 [SMILES: O=C(c1cncc(Oc2n[nH]c(=O)c(C(F)(F)F)c2N2CC3(CCC3)C2)c1)N1CCN(c2ncc(C(F)(F)F)cn2)CC1] | 611.4 | 0.5 TFA salt, ¹H NMR (400 MHz, methanol-d₄, ppm): δ 8.66-8.54 (m, 4H), 7.93-7.91 (m, 1H), 4.58 (s, 4H), 4.10- 3.97 (m, 4H), 3.93-3.80 (s, 2H), 3.65-3.54 (m, 2H), 2.27 (t, J = 7.7 Hz, 4H), 1.90-1.80 (m, 2H). 19F NMR (376 MHz, methanol-d₄, ppm): δ −53.28 (3F), −62.66 (3F). |
| 85 | 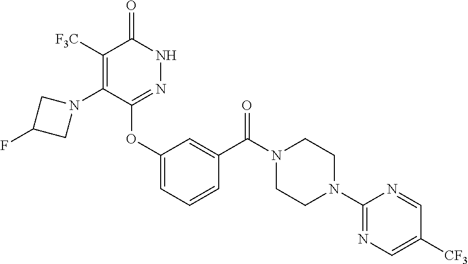 [SMILES: O=C(c1cccc(Oc2n[nH]c(=O)c(C(F)(F)F)c2N2CC(F)C2)c1)N1CCN(c2ncc(C(F)(F)F)cn2)CC1] | 588.2 | 1.6 TFA salt, ¹H NMR (400 MHz, DMSO-d₆): δ 11.71 (s, 1H), 8.74 (s, 2H), 7.54-7.50 (m, 1H), 7.38-7.30 (m, 3H), 5.47-5.29 (m, 1H), 4.82-4.70 (m, 2H), 4.55-4.44 (m, 2H), 3.89-3.65 (m, 8H); ¹⁹F NMR (376 MHz, DMSO-d₆, ppm): δ −51.65 (3F), −59.30 (3F), −178.65 (1F). |
| 86 | 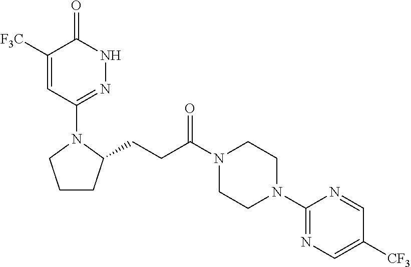 [SMILES: O=C(CC[C@@H]1CCCN1c1cc(C(F)(F)F)c(=O)[nH]n1)N1CCN(c2ncc(C(F)(F)F)cn2)CC1] | 520.3 | ¹H NMR (400 MHz, methanol-d₄, ppm): 8.59 (s, 2H), 7.86 (s, 1H), 4.03-3.85 (m, 5H), 3.72-3.59 (m, 4H), 3.56-3.49 (m, 1H), 3.35-3.31 (m, 1H), 2.55-2.44 (m, 2H), 2.17-1.85 (m, 5H), 1.75-1.61 (m, 1H). |
| 88 | 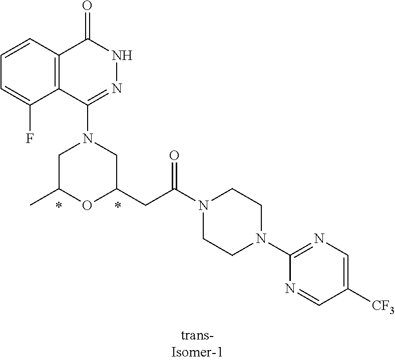 [SMILES: CC1CN(c2n[nH]c(=O)c3cccc(F)c23)CC(CC(=O)N2CCN(c3ncc(C(F)(F)F)cn3)CC2)O1] | 536.4 | SFC analysis: 100% ee; retention time: 3.079 min; column: Chiral-MIC, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.61 (s, 2H), 8.23 (d, J = 7.88 Hz, 1H), 7.91-7.86 (m, 1H), 7.72-7.66 (m, 1H), 4.54-4.52 (m, 1H), 4.27-4.23 (m, 1H), 3.98-3.93 (m, 4H), 3.78-3.71 (m, 4H), 3.29-2.72 (m, 6H), 1.31 (s, 3H). |
| 89 | 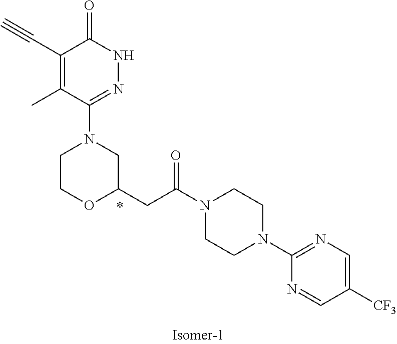 [SMILES: C#Cc1c(C)c(N2CCOC(CC(=O)N3CCN(c4ncc(C(F)(F)F)cn4)CC3)C2)n[nH]c1=O] | 492.3 | SFC analysis: 98.56% ee; retention time: 2.247 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 4.36 (s, 1H), 4.16-3.58 (m, 12H), 3.13- 3.08 (m, 1H), 2.91-2.75 (m, 2H), 2.67-2.50 (m, 2H), 2.44 (s, 3H). |
| 90 | 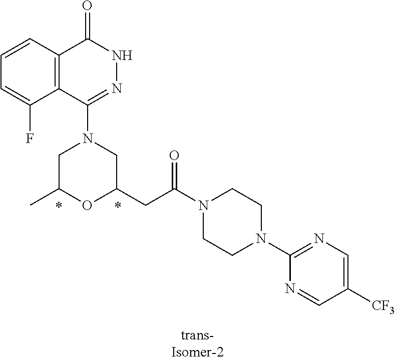 [SMILES: CC1CN(c2n[nH]c(=O)c3cccc(F)c23)CC(CC(=O)N2CCN(c3ncc(C(F)(F)F)cn3)CC2)O1] | 536.4 | SFC analysis: 100% ee; retention time: 3.091 min; column: Chiral-MIC, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.61 (s, 2H), 8.23 (d, J = 7.88 Hz, 1H), 7.91-7.86 (m, 1H), 7.72-7.67 (m, 1H), 4.54-4.52 (m, 1H), 4.27-4.23 (m, 1H), 4.00-3.93 (m, 4H), 3.77-3.68 (m, 4H), 3.29-2.72 (m, 6H), 1.31 (s, 3H). |
| 91 | 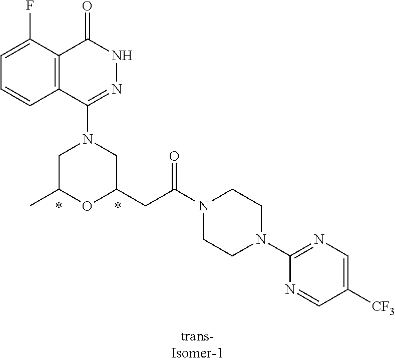 [SMILES: CC1CN(c2n[nH]c(=O)c3c(F)cccc23)CC(CC(=O)N2CCN(c3ncc(C(F)(F)F)cn3)CC2)O1] | 536.4 | SFC analysis: 100% ee; retention time: 3.342 min; column: Chiral-MIC, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.61 (s, 2H), 7.98-7.92 (m, 2H), 7.57-7.52 (m, 1H), 4.57- 4.52 (m, 1H), 4.32-4.28 (m, 1H), 4.03-3.89 (m, 4H), 3.81- 3.66 (m, 4H), 3.29-3.25 (m, 2H), 3.12-3.08 (m, 1H), 3.05- 2.95 (m, 2H), 2.88-2.84 (m, 1H), 1.37 (d, J = 6.4 Hz, 3H). |
| 92 | 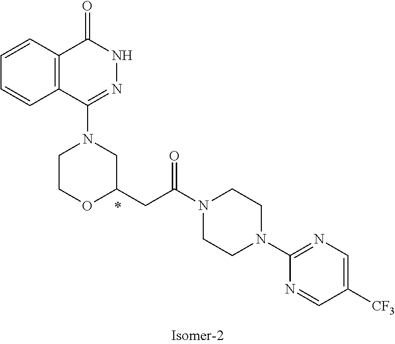 [SMILES: O=C(CC1CN(c2n[nH]c(=O)c3ccccc23)CCO1)N1CCN(c2ncc(C(F)(F)F)cn2)CC1] | 504.0 | SFC analysis: 100% ee; retention time: 4.399 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, DMSO-d₆): δ 12.13 (s, 1H), 8.71 (s, 2H), 8.22 (d, J = 7.7 Hz, 1H), 8.02-7.86 (m, 2H), 7.85- 7.81 (m, 1H), 4.16-4.10 (m, 1H), 3.90-3.76 (m, 6H), 3.59- 3.55 (m, 4H), 3.45 (d, J = 12.3 Hz, 1H), 3.27-3.24 (m, 1H), 2.87-2.80 (m, 1H), 2.73- 2.66 (m, 1H), 2.65-2.51 (m, 2H). |
| 93 | 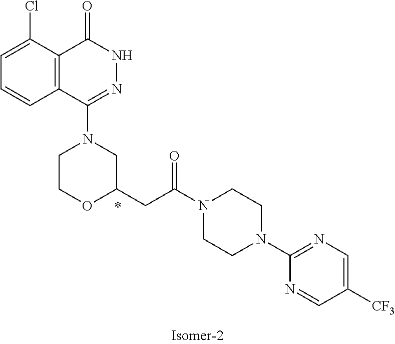 [SMILES: O=C(CC1CN(c2n[nH]c(=O)c3c(Cl)cccc23)CCO1)N1CCN(c2ncc(C(F)(F)F)cn2)CC1] | 538.1 | SFC analysis: 96.63% ee; retention time: 3.813 min; column: DAICEL CHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.08-8.00 (m, 1H), 7.87-7.75 (m, 2H), 4.30-4.19 (m, 1H), 4.07-3.83 (m, 6H), 3.80-3.59 (m, 4H), 3.50-3.40 (m, 1H), 3.30-3.22 (m, 1H), 3.03-2.92 (m, 1H), 2.90-2.70 (m, 2H), 2.61-2.51 (m, 1H). |
| 94 | 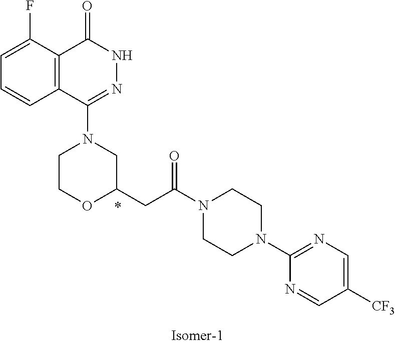 [SMILES: O=C(CC1CN(c2n[nH]c(=O)c3c(F)cccc23)CCO1)N1CCN(c2ncc(C(F)(F)F)cn2)CC1] | 522.3 | ¹H NMR (400 MHz, methanol-d₄, ppm): δ 8.59 (s, 2H), 7.98-7.83 (m, 2H), 7.57-7.49 (m, 1H), 4.31-4.19 (m, 1H), 4.28-3.80 (m, 6H), 3.81-3.58 (m, 4H), 3.52-3.45 (m, 1H), 3.29-3.27 (m, 1H), 3.05-2.94 (m, 1H), 2.88-2.72 (m, 2H), 2.66-2.55 (m, 1H). |
| 95 | 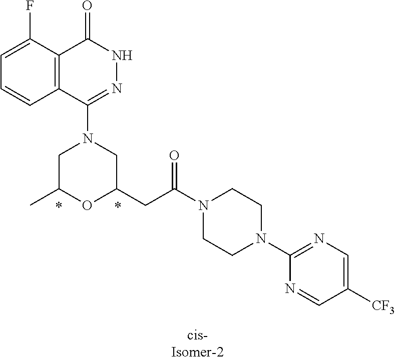 [SMILES: CC1CN(c2n[nH]c(=O)c3c(F)cccc23)CC(CC(=O)N2CCN(c3ncc(C(F)(F)F)cn3)CC2)O1] | 536.4 | SFC analysis: 74.70% ee; retention time: 3.566 min; column: DAICEL CHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 7.96-7.83 (m, 2H), 7.54-7.49 (m, 1H), 4.32-4.26 (m, 1H), 4.09-3.74 (m, 8H), 3.70-3.59 (m, 2H), 3.50-3.46 (m, 1 H), 2.86-2.81 (m, 1H), 2.71-2.52 (m, 3H), 1.19 (d, J = 6 Hz, 3H). |
| 96 | 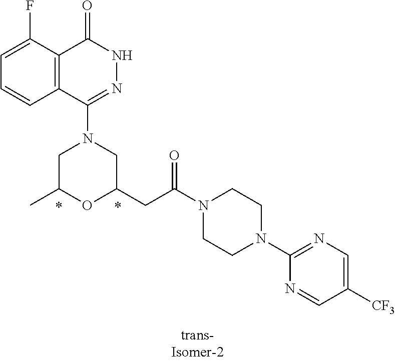 [SMILES: CC1CN(c2n[nH]c(=O)c3c(F)cccc23)CC(CC(=O)N2CCN(c3ncc(C(F)(F)F)cn3)CC2)O1] | 536.4 | SFC analysis: 100% ee; retention time: 3.451 min; column: Chiral-MIC, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.61 (s, 2H), 7.99-7.91 (m, 2H), 7.58-7.53 (m, 1H), 4.58- 4.53 (m, 1H), 4.34-4.27 (m, 1H), 4.04-3.90 (m, 4H), 3.82- 3.67 (m, 4H), 3.32-3.26 (m, 2H), 3.13-3.08 (m, 1H), 3.05- 2.95 (m, 2H), 2.89-2.84 (m, 1H), 1.38 (d, J = 6.4 Hz, 3H). |
| 97 | 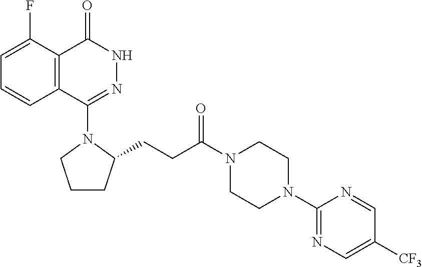 [SMILES: O=C(CC[C@@H]1CCCN1c1n[nH]c(=O)c2c(F)cccc12)N1CCN(c2ncc(C(F)(F)F)cn2)CC1] | 520.2 | ¹H NMR (400 MHz, methanol-d₄, ppm): δ 8.57 (s, 2H), 7.96-7.86 (m, 2H), 7.54-7.46 (m, 1H), 4.30-4.20 (m, 1H), 3.89-3.77 (m, 5H), 3.63-3.47 (m, 4H), 3.22-3.10 (m, 1H), 2.58-2.38 (m, 2H), 2.26-2.17 (m, 1H), 2.06-1.91 (m, 2H), 1.91-1.69 (m, 3H). |
| 98 | 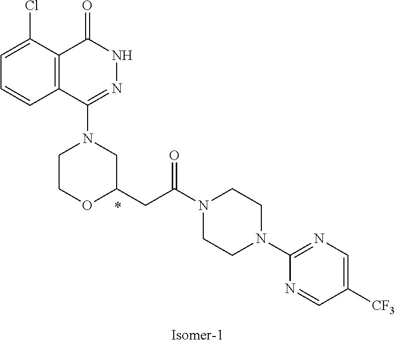 [SMILES: O=C(CC1CN(c2n[nH]c(=O)c3c(Cl)cccc23)CCO1)N1CCN(c2ncc(C(F)(F)F)cn2)CC1] | 538.1 | SFC analysis: 100% ee; retention time: 3.947 min; column: DAICEL CHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.07-8.01 (m, 1H), 7.86- 7.80 (m, 2H), 4.31-4.18 (m, 1H), 4.09-3.83 (m, 6H), 3.80- 3.58 (m, 4H), 3.51-3.41 (m, 1H), 3.32-3.23 (m, 1H), 3.05- 2.92 (m, 1H), 2.90-2.68 (m, 2H), 2.60-2.50 (m, 1H). |
| 99 | 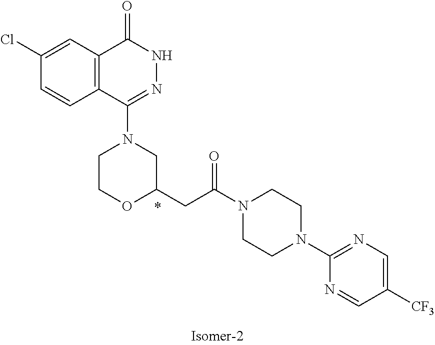 [SMILES: O=C(CC1CN(c2n[nH]c(=O)c3cc(Cl)ccc23)CCO1)N1CCN(c2ncc(C(F)(F)F)cn2)CC1] | 538.2 | SFC analysis: 99.08% ee; retention time: 4.679 min; column: DAICEL CHIPAKCEL ® OZ, 100 × 3.0 mm, 3 μm, A for CO₂ and B for MeOH (0.1% DEA), 60%; pressure: 100 bar; flow rate: 1.5 mL/min. ¹H NMR (400 MHz, methanol-d₄, ppm): δ 8.59 (s, 2H), 8.29 (d, J = 2.2 Hz, 1H), 8.06 (d, J = 8.7 Hz, 1H), 7.91 (dd, J = 8.7, 2.2 Hz, 1H), 4.28-4.22 (m, 1H), 4.05-3.85 (m, 6H), 3.80-3.62 (m, 4H), 3.55-3.50 (m, 1H), 3.36-3.28 (m, 1H), 3.05-2.96 (m, 1H), 2.87-2.74 (m, 2H), 2.61-2.53 (m, 1H). |
| 100 | 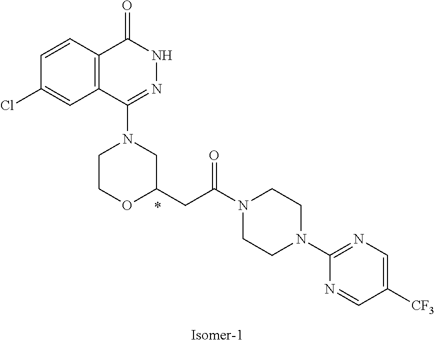 [SMILES: O=C(CC1CN(c2n[nH]c(=O)c3ccc(Cl)cc23)CCO1)N1CCN(c2ncc(C(F)(F)F)cn2)CC1] | 538.2 | SFC analysis: 99.00% ee; retention time: 4.629 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.31 (d, J = 8.5 Hz, 1H), 8.00 (d, J = 1.9 Hz, 1H), 7.83 (dd, J = 8.6, 2.0 Hz, 1H), 4.30-4.18 (m, 1H), 4.09-3.84 (m, 6H), 3.81-3.61 (m, 4H), 3.53-3.43 (m, 1H), 3.29-3.25 (m, 1H), 3.06-2.97 (m, 1H), 2.89-2.75 (m, 2H), 2.64-2.55 (m, 1H). |
| 101 | 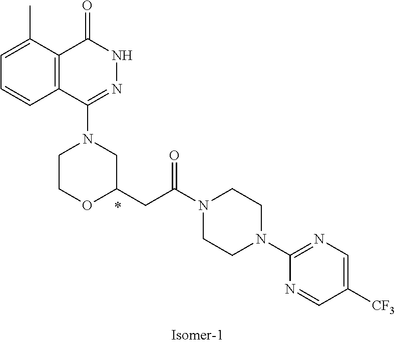 [SMILES: Cc1cccc2c(N3CCOC(CC(=O)N4CCN(c5ncc(C(F)(F)F)cn5)CC4)C3)n[nH]c(=O)c12] | 518.3 | SFC analysis: 98.32% ee; retention time: 7.275 min; column: DAICEL CHIRALPAK ® IA, 100 × 3.0 mm, 3 μm, A for CO₂ and B for MeOH (0.1% DEA), 40%; pressure: 100 bar; flow rate: 1.5 mL/min. ¹H NMR (400 MHz, methanol- d₄, ppm): δ 8.59 (s, 2H), 7.92 (d, J = 8.2 Hz, 1H), 7.82-7.71 (m, 1H), 7.58 (d, J = 7.6 Hz, 1H), 4.29-4.19 (m, 1H), 4.07- 3.83 (m, 6H), 3.81-3.61 (m, 4H), 3.46 (d, J = 12.6 Hz, 1H), 3.26-3.21 (m, 1H), 3.01-2.87 (m, 4H), 2.87-2.71 (m, 2H), 2.61-2.51 (m, 1H). |
| 102 | 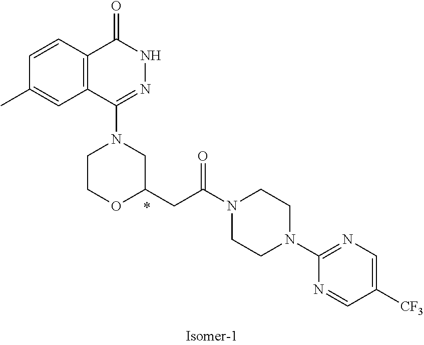 [SMILES: Cc1ccc2c(=O)[nH]nc(N3CCOC(CC(=O)N4CCN(c5ncc(C(F)(F)F)cn5)CC4)C3)c2c1] | 518.3 | SFC analysis: 98.46% ee; retention time: 2.198 min; column: DAICEL CHICEL ® OD, 250 × 4.6 mm, 5 μm, A for CO₂ and B for MeOH (0.1% DEA), 40%; pressure: 100 bar; flow rate: 3.0 mL/min. ¹H NMR (400 MHz, methanol-d₄, ppm): δ 8.59 (s, 2H), 8.22 (d, J = 8.2 Hz, 1H), 7.86 (s, 1H), 7.68 (d, J = 8.2 Hz, 1H), 4.32-4.23 (m, 1H), 4.04-3.83 (m, 6H), 3.82-3.61 (m, 4H), 3.57-3.51 (m, 1H), 3.38-3.32 (m, 1H), 3.04-2.97 (m, 1H), 2.88-2.74 (m, 2H), 2.63-2.54 (m, 4H). |
| 103 | 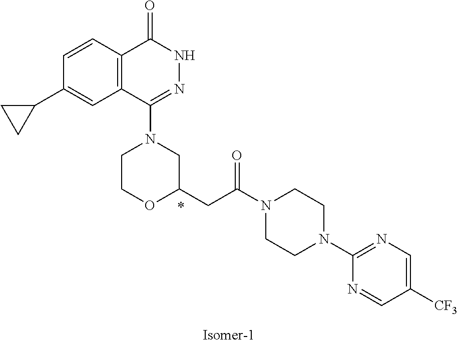 [SMILES: O=C(CC1CN(c2n[nH]c(=O)c3ccc(C4CC4)cc23)CCO1)N1CCN(c2ncc(C(F)(F)F)cn2)CC1] | 544.3 | SFC analysis: 100% ee; retention time; 4.439 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.20 (d, J = 8.4 Hz, 1H), 7.72 (s, 1H), 7.50 (d, J = 8.4 Hz, 1H), 4.34-4.22 (m, 1H), 4.07-3.84 (m, 6H), 3.80-3.61 (m, 4H), 3.57-3.51 (m, 1H), 3.35-3.32 (m, 1H), 3.07-2.97 (m, 1H), 2.87-2.70 (m, 2H), 2.63-2.54 (m, 1H), 2.23-2.15 (m, 1H), 1.20-1.14 (m, 2H), 0.97-0.85 (m, 2H). |
| 104 | 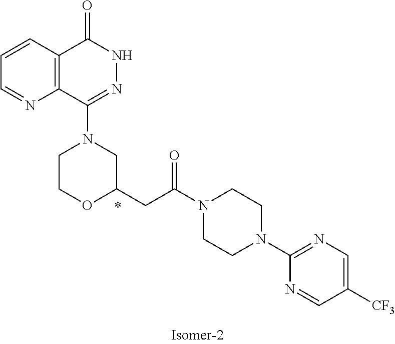 [SMILES: O=C(CC1CN(c2n[nH]c(=O)c3cccnc23)CCO1)N1CCN(c2ncc(C(F)(F)F)cn2)CC1] | 505.2 | SFC analysis: 99.38% ee; retention time: 5.265 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. 0.4 FA salt, ¹H NMR (400 MHz, DMSO-d₆): δ 12.27 (s, 1H), 9.11 (dd, J = 4.5, 1.8 Hz, 1H), 8.73 (s, 2H), 8.58 (dd, J = 8.1, 1.8 Hz, 1H), 8.43 (s, 0.4H, FA), 7.84 (dd, J = 8.0, 4.5 Hz, 1H), 4.15-3.95 (m, 3H), 3.93-3.73 (m, 6H), 3.66-3.56 (m, 4H), 2.92-2.83 (m, 1H), 2.77-2.66 (m, 2H), 2.60-2.50 (m, 1H). |
| 105 | 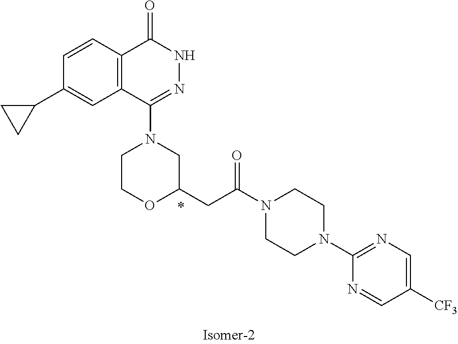 [SMILES: O=C(CC1CN(c2n[nH]c(=O)c3ccc(C4CC4)cc23)CCO1)N1CCN(c2ncc(C(F)(F)F)cn2)CC1] | 544.3 | SFC analysis: 96.08% ee; retention time: 4.063 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.20 (d, J = 8.4 Hz, 1H), 7.71 (s, 1H), 7.50 (d, J = 8.4 Hz, 1H), 4.31-4.23 (m, 1H), 4.06-3.84 (m, 6H), 3.80-3.61 (m, 4H), 3.58-3.50 (m, 1H), 3.34-3.32 (m, 1H), 3.06-2.96 (m, 1H), 2.89-2.70 (m, 2H), 2.63-2.53 (m, 1H), 2.25-2.12 (m, 1H), 1.21-1.13 (m, 2H), 0.97-0.86 (m, 2H). |
| 106 | 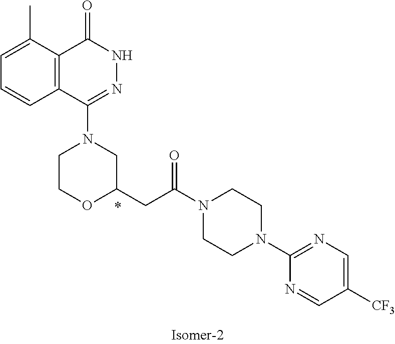 [SMILES: Cc1cccc2c(N3CCOC(CC(=O)N4CCN(c5ncc(C(F)(F)F)cn5)CC4)C3)n[nH]c(=O)c12] | 518.3 | SFC analysis: 98.32% ee; retention time: 7.275 min; column: DAICEL CHIRALPAK ® IA, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 7.88 (d, J = 8.2 Hz, 1H), 7.81-7.68 (m, 1H), 7.62-7.52 (m, 1H), 4.30-4.19 (m, 1H), 4.05-3.84 (m, 6H), 3.82-3.60 (m, 4H), 3.50-3.40 (m, 1H), 3.28-3.25 (m, 1H), 3.01-2.87 (m, 4H), 2.86-2.71 (m, 2H), 2.61-2.52 (m, 1H). |
| 107 | 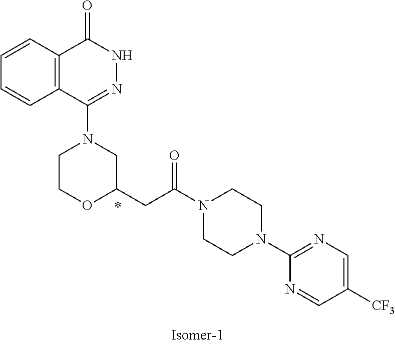 [SMILES: O=C(CC1CN(c2n[nH]c(=O)c3ccccc23)CCO1)N1CCN(c2ncc(C(F)(F)F)cn2)CC1] | 503.9 | SFC analysis: 100% ee; retention time: 4.813 min; column: DAICELCHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, DMSO- d₆): δ 12.13 (s, 1H), 8.71 (s, 2H), 8.22 (d, J = 7.7 Hz, 1H), 8.02-7.86 (m, 2H), 7.85-7.81 (m, 1H), 4.16-4.10 (m, 1H), 3.91-3.78 (m, 6H), 3.59-3.55 (m, 4H), 3.45 (d, J = 12.3 Hz, 1H), 3.30-3.24 (m, 1H), 2.87-2.80 (m, 1H), 2.73-2.66 (m, 1H), 2.65-2.51 (m, 2H). |
| 109 | 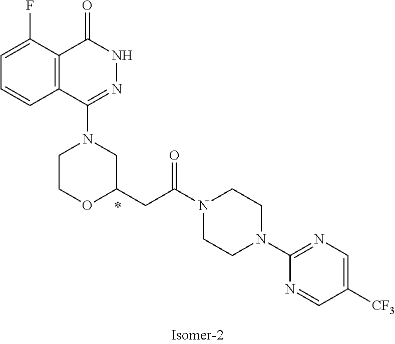 [SMILES: O=C(CC1CN(c2n[nH]c(=O)c3c(F)cccc23)CCO1)N1CCN(c2ncc(C(F)(F)F)cn2)CC1] | 522.3 | ¹H NMR (400 MHz, methanol-d₄, ppm): δ 8.59 (s, 2H), 8.01-7.79 (m, 2H), 7.62-7.37 (m, 1H), 4.32-4.16 (m, 1H), 4.09-3.82 (m, 6H), 3.83-3.60 (m, 4H), 3.52-3.46 (m, 1H), 3.30-3.26 (m, 1H), 3.04-2.92 (m, 1H), 2.87-2.71 (m, 2H), 2.67-2.54 (m, 1H). |
| 110 | 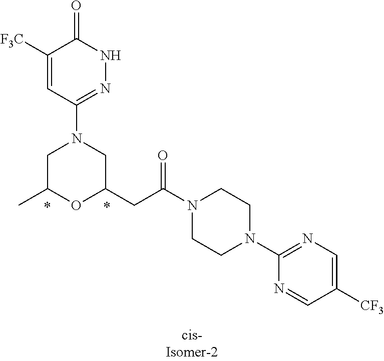 [SMILES: CC1CN(c2cc(C(F)(F)F)c(=O)[nH]n2)CC(CC(=O)N2CCN(c3ncc(C(F)(F)F)cn3)CC2)O1] | 536.0 | SFC analysis: 99.30% ee; retention time: 2.782 min; column: DAICELCHIRALPAK ® OJ, 100 × 3.0 mm, 3 μm, A for CO₂ and B for MeOH (0.1% DEA), 40%; pressure: 100 bar; flow rate: 1.5 mL/min. ¹H NMR (400 MHz, methanol- d₄, ppm): δ 8.59 (s, 2H), 7.88 (s, 1H), 4.08-3.98 (m, 3H), 3.94-3.59 (m, 9H), 2.86-2.80 (m, 1H), 2.63-2.45 (m, 3H), 1.19 (d, J = 6.2 Hz, 3H). |
| 111 | 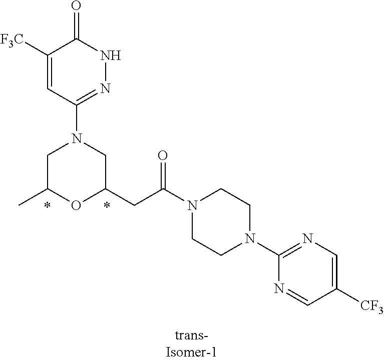 [SMILES: CC1CN(c2cc(C(F)(F)F)c(=O)[nH]n2)CC(CC(=O)N2CCN(c3ncc(C(F)(F)F)cn3)CC2)O1] | 536.0 | SFC analysis: 88.44% ee; retention time: 1.585 min; column: Chiral-MIC, 100 × 3.0 mm, 3 μm, A for CO₂ and B for MeOH (0.1% DEA), 20% to 40%; pressure: 100 bar; flow rate: 1.5 mL/min. ¹H NMR (400 MHz, methanol-d₄, ppm): δ 8.59 (s, 2H), 7.84 (s, 1H), 4.44-4.38 (m, 1H), 4.16-4.08 (m, 1H), 4.02-3.87 (m, 4H), 3.76-3.65 (m, 4H), 3.52- 3.47 (m, 1H), 3.41-3.34 (m, 2H), 2.99-2.94 (m, 1H), 2.90-2.79 (m, 2H), 1.24 (d, J = 6.4 Hz, 3H). |
| 112 | 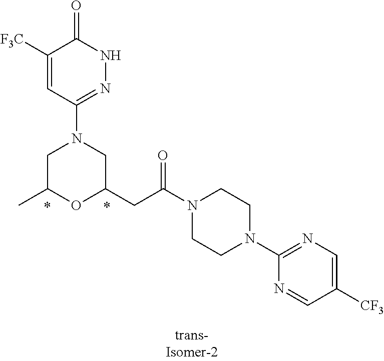 [SMILES: CC1CN(c2cc(C(F)(F)F)c(=O)[nH]n2)CC(CC(=O)N2CCN(c3ncc(C(F)(F)F)cn3)CC2)O1] | 536.0 | SFC analysis: 99.32% ee; retention time: 3.120 min; column: Chiral-MIC, 100 × 3.0 mm, 3 μm, A for CO₂ and B for MeOH (0.1% DEA), 20% to 40%; pressure: 100 bar; flow rate: 1.5 mL/min. ¹H NMR (400 MHz, methanol-d₄, ppm): δ 8.59 (s, 2H), 7.84 (s, 1H), 4.43-4.39 (m, 1H), 4.14- 4.09 (m, 1H), 4.00-3.88 (m, 4H), 3.77-3.65 (m, 4H), 3.51- 3.47 (m, 1H), 3.41-3.34 (m, 2H), 2.99-2.94 (m, 1H), 2.90- 2.79 (m, 2H), 1.24 (d, J = 6.4 Hz, 3H). |
| 113 | 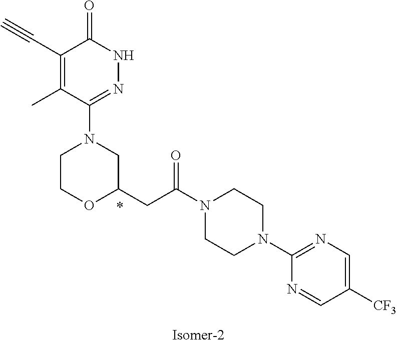 [SMILES: C#Cc1c(C)c(N2CCOC(CC(=O)N3CCN(c4ncc(C(F)(F)F)cn4)CC3)C2)n[nH]c1=O] | 492.3 | SFC analysis: 88.10% ee; retention time: 2.598 min; column: Regis WHELK-O1(S, S), 50 × 4.6 mm, 3 μm, A for CO₂ and B for MeOH (0.1% DEA), 40%; pressure; 100 bar; flow rate: 1.5 mL/min. ¹H NMR (400 MHz, methanol-d₄, ppm): δ 8.59 (s, 2H), 4.36 (s, 1H), 4.13-3.60 (m, 12H), 3.13- 3.07 (m, 1H), 2.88-1.78 (m, 2H), 2.67-2.51 (m, 2H), 2.44 (s, 3H). |
| 114 | 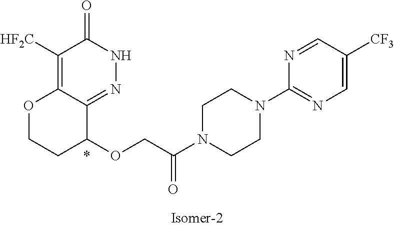 | 491.4 | SFC analysis: 100% ee; retention time: 13.739 min; column: Daicel CHIRALPAK ® AY, 250 × 4.6 mm, 5 μm, A for n-hexane and B for EtOH (0.2% DEA), 40%; pressure: 100 bar; flow rate: 1.0 mL/min. ¹H NMR (400 MHz, DMSO-d₆): δ 13.16 (s, 1H), 8.72 (s, 2H), 6.89 (t, J = 53.2 Hz, 1H), 4.53-4.30 (m, 5H), 3.91- 3.70 (m, 4H), 3.55-3.45 (m, 4H), 2.34-2.13 (m, 2H). |
| 115 | 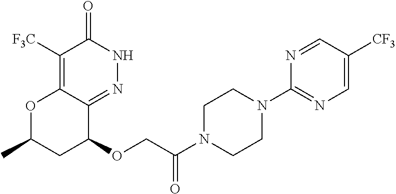 [SMILES: C[C@@H]1C[C@H](OCC(=O)N2CCN(c3ncc(C(F)(F)F)cn3)CC2)c2n[nH]c(=O)c(C(F)(F)F)c2O1] | 523.4 | ¹H NMR (400 MHz, DMSO-d₆): δ 13.23 (s, 1H), 8.74 (s, 2H), 4.75-4.60 (m, 2H), 4.57-4.41 (m, 2H), 3.95-3.75 (m, 4H), 3.63-3.45 (m, 4H), 2.53-2.30 (m, 1H), 2.03-1.88 (m, 1H), 1.43 (d, J = 6.4 Hz, 3H). |
| 116 | 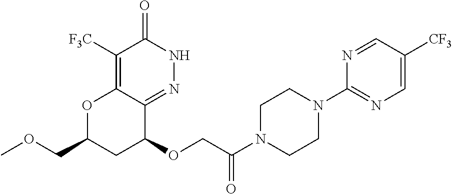 [SMILES: COC[C@@H]1C[C@H](OCC(=O)N2CCN(c3ncc(C(F)(F)F)cn3)CC2)c2n[nH]c(=O)c(C(F)(F)F)c2O1] | 553.2 | ¹H NMR (400 MHz, DMSO-d₆): δ 13.25 (s, 1H), 8.74 (s, 2H), 4.75-4.70 (m, 2H), 4.52-4.42 (m, 2H), 3.87-3.72 (m, 5H), 3.62-3.58 (m, 5H), 3.31 (s, 3H), 2.49-2.44 (m, 1H), 2.11-2.04 (m, 1H). |
| 118 | 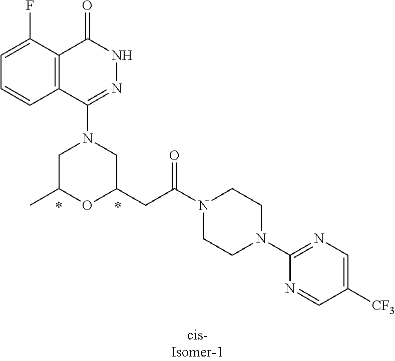 [SMILES: CC1CN(c2n[nH]c(=O)c3c(F)cccc23)CC(CC(=O)N2CCN(c3ncc(C(F)(F)F)cn3)CC2)O1] | 536.4 | SFC analysis: 92.96% ee; retention time: 3.223 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 7.96-7.87 (m, 2H), 7.54-7.49 (m, 1H), 4.32-4.26 (m, 1H), 4.09-3.74 (m, 8H), 3.70-3.59 (m, 2H), 3.50-3.47 (m, 1H), 2.86-2.81 (m, 1H), 2.71-2.52 (m, 3H), 1.18 (d, J = 6 Hz, 3H). |
| 119 | 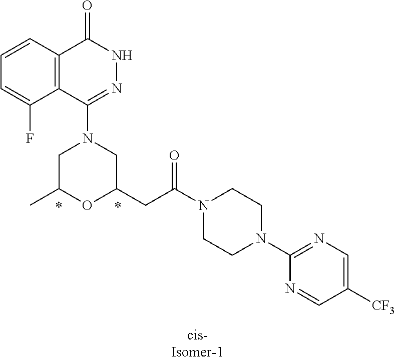 [SMILES: CC1CN(c2n[nH]c(=O)c3cccc(F)c23)CC(CC(=O)N2CCN(c3ncc(C(F)(F)F)cn3)CC2)O1] | 536.4 | SFC analysis: 97.06% ee; retention time: 3.075 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.21 (d, J = 8.0 Hz, 1H), 7.88-7.83 (m, 1H), 7.68-7.63 (m, 1H), 4.24-4.22 (m, 1H), 4.10- 3.99 (m, 2H), 3.94-3.75 (m, 6H), 3.71-3.58 (m, 2H), 3.48-3.45 (m, 1H), 2.86-2.81 (m, 1H), 2.69-2.63 (m, 1H), 2.56-2.49 (m, 2H), 1.18 (d, J = 6 Hz, 3H). |
| 120 | 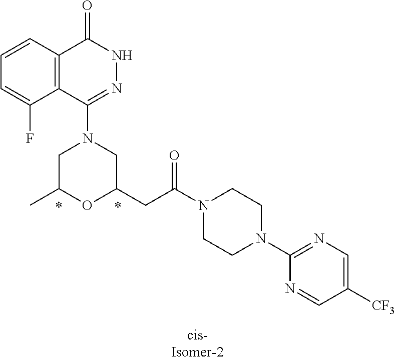 [SMILES: CC1CN(c2n[nH]c(=O)c3cccc(F)c23)CC(CC(=O)N2CCN(c3ncc(C(F)(F)F)cn3)CC2)O1] | 536.4 | SFC analysis: 98.98% ee; retention time: 3.452 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol- d₄, ppm): δ 8.59 (s, 2H), 8.20 (d, J = 8.0 Hz, 1H), 7.88-7.83 (m, 1H), 7.68-7.63 (m, 1H), 4.26-4.22 (m, 1H), 4.10-3.99 (m, 2H), 3.94-3.75 (m, 6H), 3.71-3.58 (m, 2H), 3.48-3.45 (m, 1H), 2.86-2.81 (m, 1H), 2.69-2.63 (m, 1H), 2.56-2.49 (m, 2H), 1.19 (d, J = 6.4 Hz, 3H). |
| 121 | 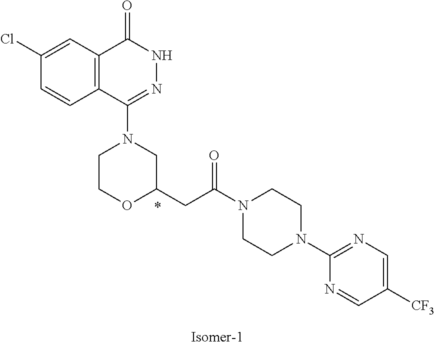 [SMILES: O=C(CC1CN(c2n[nH]c(=O)c3cc(Cl)ccc23)CCO1)N1CCN(c2ncc(C(F)(F)F)cn2)CC1] | 538.3 | SFC analysis: 97.94% ee; retention time: 5.217 min; column: DAICEL CHIPAKCEL ® OZ, 100 × 3.0 mm, 3 μm, A for CO₂ and B for MeOH (0.1% DEA), 60%; pressure: 100 bar; flow rate: 1.5 mL/min. ¹H NMR (400 MHz, methanol-d₄, ppm): δ 8.59 (s, 2H), 8.28 (d, J = 2.1 Hz, 1H), 8.06 (d, J = 8.7 Hz, 1H), 7.91 (dd, J = 8.7, 2.2 Hz, 1H), 4.29-4.21 (m, 1H), 4.05-3.86 (m, 6H), 3.79-3.63 (m, 4H), 3.53-3.48 (m, 1H), 3.36-3.32 (m, 1H), 3.05-2.96 (m, 1H), 2.87-2.73 (m, 2H), 2.60-2.55 (m, 1H). |
| 122 | 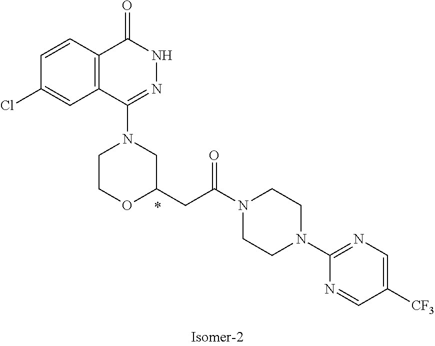 [SMILES: O=C(CC1CN(c2n[nH]c(=O)c3ccc(Cl)cc23)CCO1)N1CCN(c2ncc(C(F)(F)F)cn2)CC1] | 538.2 | SFC analysis: 98.04% ee; retention time: 4.251 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.31 (d, J = 8.6 Hz, 1H), 8.00 (d, J = 2.0 Hz, 1H), 7.84 (dd, J = 8.6, 2.0 Hz, 1H), 4.30-4.22 (m, 1H), 4.07-3.84 (m, 6H), 3.81-3.62 (m, 4H), 3.51-3.45 (m, 1H), 3.29-3.27 (m, 1H), 3.06-2.96 (m, 1H), 2.88-2.77 (m, 2H), 2.63-2.55 (m, 1H). |
| 123 | 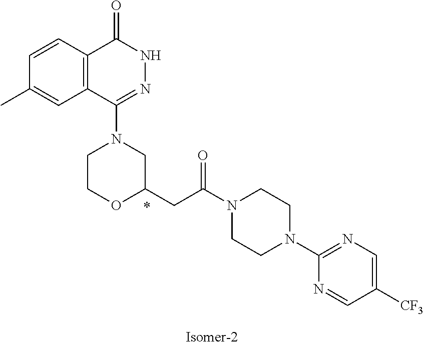 [SMILES: Cc1ccc2c(=O)[nH]nc(N3CCOC(CC(=O)N4CCN(c5ncc(C(F)(F)F)cn5)CC4)C3)c2c1] | 518.3 | SFC analysis: 97.28% ee; retention time: 2.554 min; column: DAICEL CHICEL ® OD, 250 × 4.6 mm, 5 μm, A for CO₂ and B for MeOH (0.1% DEA), 40%; pressure: 100 bar; flow rate: 3.0 mL/mmin. ¹H NMR (400 MHz, methanol-d₄, ppm): δ 8.59 (s, 2H), 8.22 (d, J = 8.1 Hz, 1H), 7.85 (s, 1H), 7.68 (d, J = 8.2 Hz, 1H), 4.32-4.21 (m, 1H), 4.07-3.85 (m, 6H), 3.82-3.61 (m, 4H), 3.57-3.51 (m, 1H), 3.38-3.32 (m, 1H), 3.05-2.96 (m, 1H), 2.89-2.73 (m, 2H), 2.65-2.53 (m, 4H). |
| 124 | 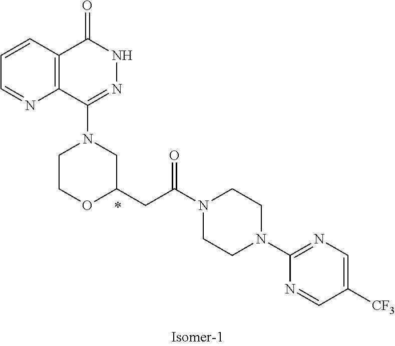 [SMILES: O=C(CC1CN(c2n[nH]c(=O)c3cccnc23)CCO1)N1CCN(c2ncc(C(F)(F)F)cn2)CC1] | 505.2 | SFC analysis: 99.08% ee; retention time: 4.747 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. 0.14 FA salt, ¹H NMR (400 MHz, DMSO-d₆): δ 12.27 (s, 1H), 9.11 (dd, J = 4.5, 1.7 Hz, 1H), 8.73 (s, 2H), 8.58 (dd, J = 8.0, 1.8 Hz, 1H), 8.46 (s, 0.14H, FA), 7.84 (dd, J = 8.1, 4.5 Hz, 1H), 4.20- 3.95 (m, 3H), 3.93-3.72 (m, 6H), 3.65-3.55 (m, 4H), 2.95- 2.83 (m, 1H), 2.78-2.66 (m, 2H), 2.61-2.50 (m, 1H). |
| 125 | 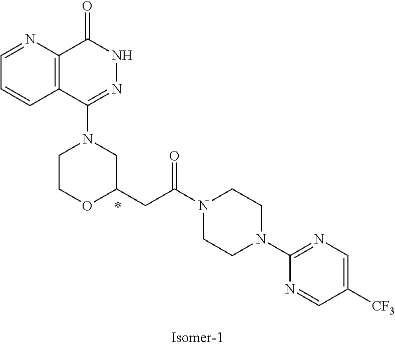 [SMILES: O=C(CC1CN(c2n[nH]c(=O)c3ncccc23)CCO1)N1CCN(c2ncc(C(F)(F)F)cn2)CC1] | 505.3 | ¹H NMR (400 MHz, methanol-d₄, ppm): δ 9.06 (s, 1H), 8.59 (s, 2H), 8.49 (d, J = 8.3 Hz, 1H), 7.92 (dd, J = 8.2, 4.4 Hz, 1H), 4.30-4.22 (m, 1H), 4.10-3.84 (m, 6H), 3.82-3.60 (m, 4H), 3.59-3.48 (m, 1H), 3.38-3.32 (m, 1H), 3.09-2.98 (m, 1H), 2.88-2.76 (m, 2H), 2.65-2.55 (m, 1H). |
| 126 | 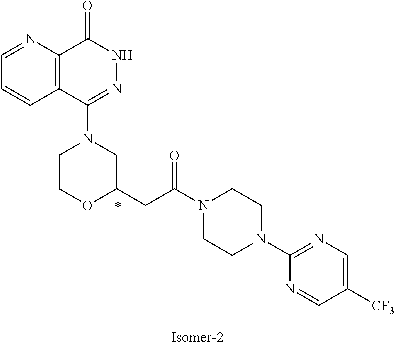 [SMILES: O=C(CC1CN(c2n[nH]c(=O)c3ncccc23)CCO1)N1CCN(c2ncc(C(F)(F)F)cn2)CC1] | 505.3 | ¹H NMR (400 MHz, methanol-d₄, ppm): δ 9.05 (s, 1H), 8.59 (s, 2H), 8.49 (d, J = 8.2 Hz, 1H), 7.92 (dd, J = 8.1, 4.4 Hz, 1H), 4.31-4.22 (m, 1H), 4.07-3.84 (m, 6H), 3.81-3.61 (m, 4H), 3.60-3.49 (m, 1H), 3.37-3.32 (m, 1H), 3.09-2.96 (m, 1H), 2.88-2.76 (m, 2H), 2.65-2.54 (m, 1H). |
| 127 | 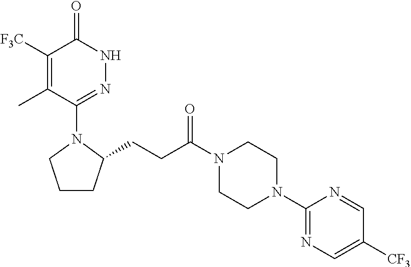 [SMILES: Cc1c(N2CCC[C@H]2CCC(=O)N2CCN(c3ncc(C(F)(F)F)cn3)CC2)n[nH]c(=O)c1C(F)(F)F] | 534.3 | ¹H NMR (400 MHz, methanol-d₄, ppm): δ 8.59 (s, 2H), 4.09-4.01 (m, 1H), 3.99-3.83 (m, 4H), 3.71-3.52 (m, 5H), 3.00-2.89 (m, 1H), 2.84-2.40 (m, 4H), 2.23-2.15 (m, 1H), 2.03-1.74 (m, 3H), 1.75-1.63 (m, 2H). |
| 128 | 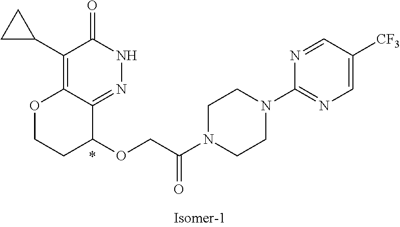 [SMILES: O=C(COC1CCOc2c1n[nH]c(=O)c2C1CC1)N1CCN(c2ncc(C(F)(F)F)cn2)CC1] | 481.4 | SFC analysis: 100% ee; retention time: 1.460 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, DMSO-d₆): δ 12.65 (s, 1H), 8.74 (s, 2H), 4.44-4.40 (m, 1H), 4.39-4.23 (m, 4H), 3.88-3.76 (m, 4H), 3.52-3.40 (m, 4H), 2.25-2.20 (m, 1H), 2.16-2.06 (m, 1H), 1.97-1.87 (m, 1H), 1.45-1.35 (m, 2H), 0.75-0.65 (m, 2H). |
| 129 | 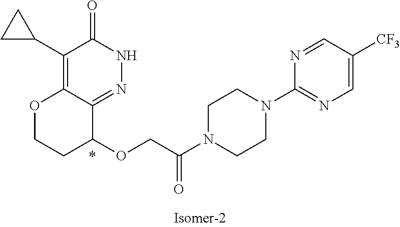 [SMILES: O=C(COC1CCOc2c1n[nH]c(=O)c2C1CC1)N1CCN(c2ncc(C(F)(F)F)cn2)CC1] | 481.4 | SFC analysis: 99.72% ee; retention time: 1.986 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, DMSO-d₆): δ 12.61 (s, 1H), 8.71 (s, 2H), 4.50-4.40 (m, 1H), 4.39-4.23 (m, 4H), 3.85-3.75 (m, 4H), 3.50-3.40 (m, 4H), 2.30-2.10 (m, 1H), 2.12-2.03 (m, 1H), 1.92-1.84 (m, 1H), 1.40-1.30 (m, 2H), 0.70-0.60 (m, 2H). |
| 130 | 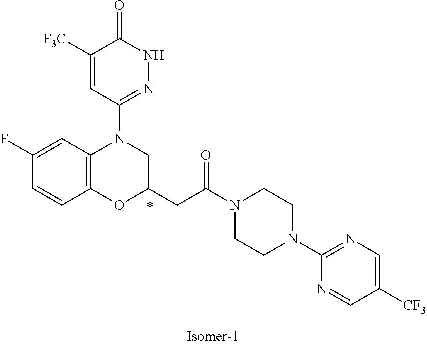 [SMILES: O=C(CC1CN(c2cc(C(F)(F)F)c(=O)[nH]n2)c2cc(F)ccc2O1)N1CCN(c2ncc(C(F)(F)F)cn2)CC1] | 588.4 | SFC analysis: 99.66% ee; retention time: 1.353 min; column: Regis WHELK-O1(S, S), 50 × 4.6 mm, 3 μm, A for CO₂ and B for MeOH (0.1% DEA), 40%; pressure: 100 bar; flow rate: 1.5 mL/ min. ¹H NMR (400 MHz, methanol-d₄, ppm): δ 8.50 (s, 2H), 7.90 (s, 1H), 6.80-6.73 (m, 2H), 6.58- 6.53 (m, 1H), 4.58-4.50 (m, 1H), 4.03-3.99 (m, 1H), 3.89- 3.82 (m, 4H), 3.67-3.53 (m, 4H), 3.45-3.40 (m, 1H), 2.94- 2.88 (m, 1H), 2.72-2.65 (m, 1H). |
| 131 | 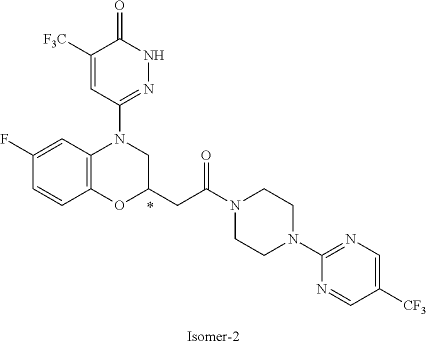 [SMILES: O=C(CC1CN(c2cc(C(F)(F)F)c(=O)[nH]n2)c2cc(F)ccc2O1)N1CCN(c2ncc(C(F)(F)F)cn2)CC1] | 588.4 | SFC analysis: 97.04% ee; retention time: 1.771 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.50 (s, 2H), 7.90 (s, 1H), 6.79-6.73 (m, 2H), 6.58- 6.53 (m, 1H), 4.58-4.51 (m, 1H), 4.03-3.99 (m, 1H), 3.89- 3.82 (m, 4H), 3.67-3.53 (m, 4H), 3.45-3.40 (m, 1H), 2.94- 2.88 (m, 1H), 2.74-2.66 (m, 1H). |
| 132 | 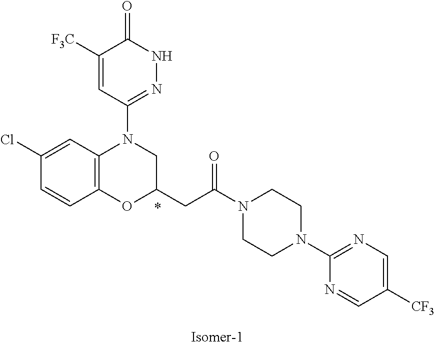 [SMILES: O=C(CC1CN(c2cc(C(F)(F)F)c(=O)[nH]n2)c2cc(Cl)ccc2O1)N1CCN(c2ncc(C(F)(F)F)cn2)CC1] | 604.4 | SFC analysis: 100% ee; retention time: 1.548 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.50 (s, 2H), 7.89 (s, 1H), 6.99-6.98 (m, 1H), 6.81-6.76 (m, 2H), 4.60-4.54 (m, 1H), 4.03-3.99 (m, 1H), 3.89-3.82 (m, 4H), 3.66-3.54 (m, 4H), 3.45-3.40 (m, 1H), 2.94-2.88 (m, 1H), 2.72-2.67 (m, 1H). |
| 133 | 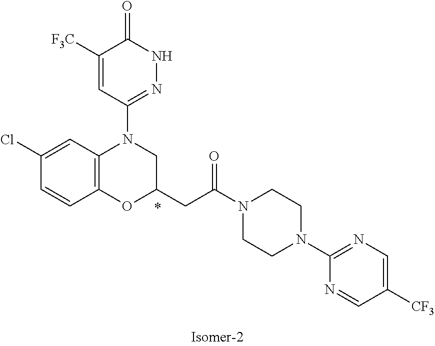 [SMILES: O=C(CC1CN(c2cc(C(F)(F)F)c(=O)[nH]n2)c2cc(Cl)ccc2O1)N1CCN(c2ncc(C(F)(F)F)cn2)CC1] | 604.4 | SFC analysis: 97.02% ee; retention time: 2.001 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.55 (s, 2H), 7.95 (s, 1H), 7.05-7.04 (m, 1H), 6.87- 6.81 (m, 2H), 4.66-4.60 (m, 1H), 4.09-4.05 (m, 1H), 3.95- 3.88 (m, 4H), 3.72-3.61 (m, 4H), 3.51-3.45 (m, 1H), 3.00- 2.94 (m, 1H), 2.78-2.72 (m, 1H). |
| 134 | 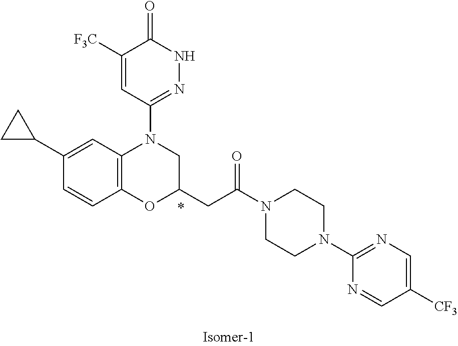 [SMILES: O=C(CC1CN(c2cc(C(F)(F)F)c(=O)[nH]n2)c2cc(C3CC3)ccc2O1)N1CCN(c2ncc(C(F)(F)F)cn2)CC1] | 610.4 | SFC analysis: 99.16% ee; retention time: 1.824 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆): δ 13.06 (s, 1H), 8.74 (s, 2H), 7.95 (s, 1H), 6.78-6.71 (m, 2H), 6.66-6.63 (m, 1H), 4.63-4.55 (m, 1H), 4.05-3.98 (m, 1H), 3.93-3.75 (m, 4H), 3.95-3.53 (m, 4H), 3.45-3.35 (m, 1H), 2.95-2.86 (m, 1H), 2.84-2.73 (m, 1H), 1.84-1.74 (m, 1H), 0.88-0.81 (m, 2H), 0.58-0.45 (m, 2H). |
| 135 | 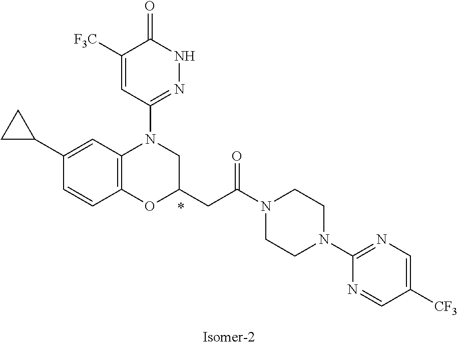 [SMILES: O=C(CC1CN(c2cc(C(F)(F)F)c(=O)[nH]n2)c2cc(C3CC3)ccc2O1)N1CCN(c2ncc(C(F)(F)F)cn2)CC1] | 610.4 | SFC analysis: 99.14% ee; retention time: 2.599 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆): δ 13.04 (s, 1H), 8.74 (s, 2H), 7.95 (s, 1H), 6.79-6.71 (m, 2H), 6.68-6.62 (m, 1H), 4.63-4.55 (m, 1H), 4.05-3.98 (m, 1H), 3.96-3.74 (m, 4H), 3.59-3.53 (m, 4H), 3.49-3.42 (m, 1H), 2.95-2.86 (m, 1H), 2.84-2.73 (m, 1H), 1.83-1.77 (m, 1H), 0.89-0.81 (m, 2H), 0.61-0.44 (m, 2H). |
| 136 | 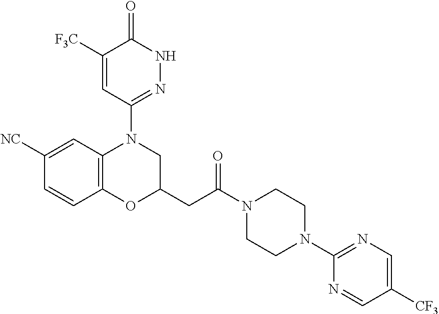 [SMILES: N#Cc1ccc2c(c1)N(c1cc(C(F)(F)F)c(=O)[nH]n1)CC(CC(=O)N1CCN(c3ncc(C(F)(F)F)cn3)CC1)O2] | 595.4 | ¹H NMR (400 MHz, DMSO-d₆): δ 8.74 (s, 2H), 8.01 (s, 1H), 7.65 (s, 1H), 7.32 (dd, J = 8.4, 1.9 Hz, 1H), 7.03 (d, J = 8.4 Hz, 1H), 4.77-4.71 (m, 1H), 4.09-4.01 (m, 1H), 3.93- 3.77 (m, 4H), 3.61-3.57 (m, 4H), .354-3.45 (m, 1H), 2.99- 2.93 (m, 1H), 2.84-2.77 (m, 1H). |
| 137 | 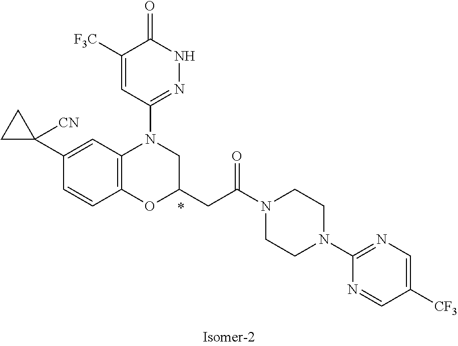 [SMILES: N#CC1(c2ccc3c(c2)N(c2cc(C(F)(F)F)c(=O)[nH]n2)CC(CC(=O)N2CCN(c4ncc(C(F)(F)F)cn4)CC2)O3)CC1] | 635.2 | SFC analysis: 98.66% ee; reetention time: 4.072 min; column: DAICEL CHIRALPAK ® ID, 100 × 3 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 8.01 (s, 1H), 7.05 (d, J = 1.6 Hz, 1H), 6.97-6.85 (m, 2H), 4.75-4.64 (m, 1H), 4.15 (dd, J = 13.2, 2.6 Hz, 1H), 4.03-3.85 (m, 4H), 3.78- 3.59 (m, 4H), 3.54 (dd, J = 13.2, 7.6 Hz, 1H), 3.06-2.96 (m, 1H), 2.84-2.74 (m, 1H), 1.65-1.55 (m, 2H), 1.44-1.27 (m, 2H). |
| 138 | 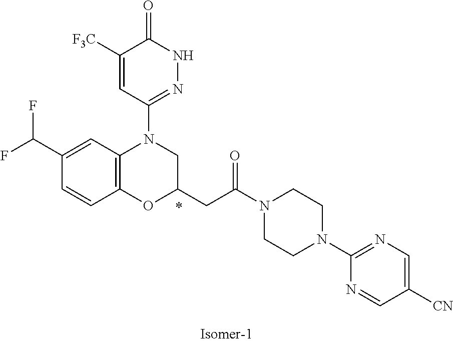 [SMILES: N#Cc1cnc(N2CCN(C(=O)CC3CN(c4cc(C(F)(F)F)c(=O)[nH]n4)c4cc(C(F)F)ccc4O3)CC2)nc1] | 577.2 | SFC analysis: 99.52% ee; retention time: 4.394 min; column: Chiral-MIC, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.99 (s, 1H), 7.23 (s, 1H), 7.12 (d, J = 8.7 Hz, 1H), 6.99 (d, J = 8.5 Hz, 1H), 6.63 (t, J = 56.4 Hz, 1H), 4.80-4.68 (m, 1H), 4.16 (dd, J = 13.3, 2.6 Hz, 1H), 4.08-3.90 (m, 4H), 3.80-3.64 (m, 4H), 3.56 (dd, J = 13.3, 7.6 Hz, 2H), 3.08- 2.98 (m, 1H), 2.88-2.76 (m, 1H). |
| 140 | 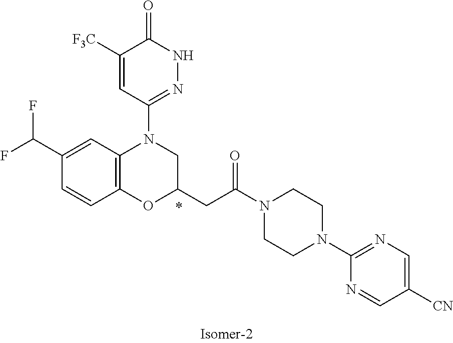 [SMILES: N#Cc1cnc(N2CCN(C(=O)CC3CN(c4cc(C(F)(F)F)c(=O)[nH]n4)c4cc(C(F)F)ccc4O3)CC2)nc1] | 577.2 | SFC analysis: 94.54% ee; retention time: 5.402 min; column: Chiral-MIC, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.99 (s, 1H), 7.23 (s, 1H), 7.11 (d, J = 8.1 Hz, 1H), 6.99 (d, J = 8.3 Hz, 1H), 6.63 (t, J = 56.6 Hz, 1H), 4.80-4.68 (m, 1H), 4.21-4.11 (m, 1H), 4.07-3.89 (m, 4H), 3.81-3.61 (m, 4H), 3.56 (dd, J = 13.3, 7.4 Hz, 1H), 3.09-2.95 (m, 1H), 2.87-2.75 (m, 1H). |
| 141 | 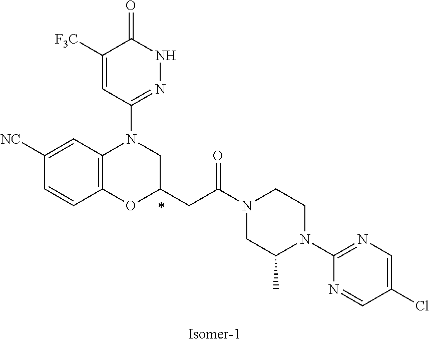 [SMILES: C[C@@H]1CN(C(=O)CC2CN(c3cc(C(F)(F)F)c(=O)[nH]n3)c3cc(C#N)ccc3O2)CCN1c1ncc(Cl)cn1] | 575.2 | SFC analysis: 100% ee; retention time: 1.393 min; column: DAICEL CHIRALPAK ® IB, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.32 (s, 2H), 8.02-7.98 (m, 1H), 7.52 (s, 1H), 7.26 (d, J = 8.5 Hz, 1H), 7.01 (dd, J = 8.4, 5.9 Hz, 1H), 4.93-4.73 (m, 2H), 4.53-4.29 (m, 2H), 4.20-4.08 (m, 1H), 4.12-4.84 (m, 1H), 3.69-3.43 (m, 1.5 H), 3.30-3.15 (m, 1.5 H), 3.10-2.75 (m, 3H), 1.25-1.10 (m, 3H). |
| 142 | 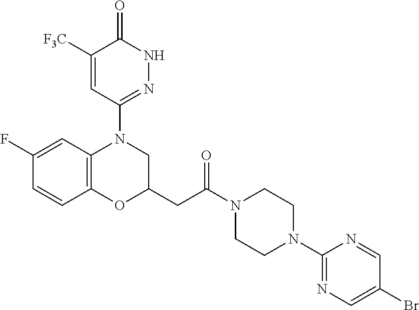 [SMILES: O=C(CC1CN(c2cc(C(F)(F)F)c(=O)[nH]n2)c2cc(F)ccc2O1)N1CCN(c2ncc(Br)cn2)CC1] | 598.1 | 0.2FA salt, ¹H NMR (400 MHz, DMSO-d₆, ppm): δ 13.01 (s, 1H), 8.49 (s, 2H), 8.42 (s, 0.2 H), 7.99 (s, 1H), 7.01 (dd, J = 10.6, 2.8 Hz, 1H), 6.95-6.85 (m, 1H), 6.73-6.65 (m, 1H), 4.64-4.54 (m, 1H), 4.04-3.94 (m, 1H), 3.84-3.64 (m, 4H), 3.62-3.51 (m, 4H), 3.46 (dd, J = 13.1, 7.6 Hz, 1H), 2.98- 2.84 (m, 1H), 2.82-2.70 (m, 1H). |
| 143 | 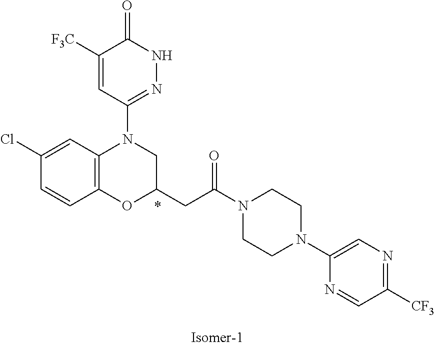 [SMILES: O=C(CC1CN(c2cc(C(F)(F)F)c(=O)[nH]n2)c2cc(Cl)ccc2O1)N1CCN(c2cnc(C(F)(F)F)cn2)CC1] | 604.2 | SFC analysis: 100% ee; retention time: 3.871 min; column: DAICEL CHIRALCEL ® AS, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.41 (s, 1H), 8.30 (s, 1H), 7.99 (s, 1H), 7.09 (d, J = 1.8 Hz, 1H), 6.93-6.83 (m, 2H), 4.73-4.62 (m, 1H), 4.11 (dd, J = 13.2, 2.4 Hz, 1H), 3.90-3.65 (m, 8H), 3.53 (dd, J = 13.2, 7.6 Hz, 1H), 3.08-2.95 (m, 1H), 2.86-2.73 (m, 1H). |
| 144 | 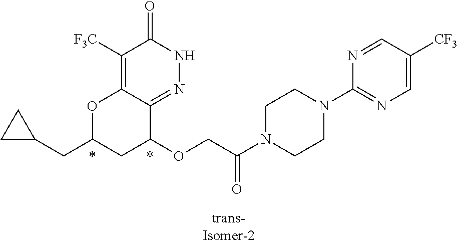 [SMILES: O=C(COC1CC(CC2CC2)Oc2c1n[nH]c(=O)c2C(F)(F)F)N1CCN(c2ncc(C(F)(F)F)cn2)CC1] | 563.4 | SFC analysis: 93.26% ee; retention time: 1.242 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 13.25 (s, 1H), 8.75 (s, 2H), 4.70-4.62 (m, 1H), 4.50 (t, J = 2.8 Hz, 1H), 4.38 (s, 2H), 3.96-3.68 (m, 4H), 3.58-3.40 (m, 4H), 2.43-2.37 (m, 1H), 2.10-1.96 (m, 1H), 1.77-1.69 (m, 1H), 1.55-1.49 (m, 1H), 0.90-0.85 (m, 1H), 0.50-0.41 (m, 2H), 0.20-0.09 (m, 2H). ¹⁹F NMR (376 MHz, DMSO-d₆, ppm): |
| | | | δ −57.37 (3F), −59.30 (3F). |
| 145 | 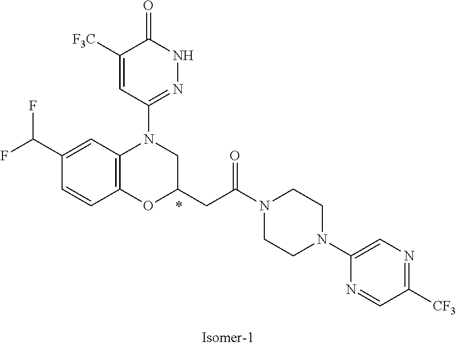 [SMILES: O=C(CC1CN(c2cc(C(F)(F)F)c(=O)[nH]n2)c2cc(C(F)F)ccc2O1)N1CCN(c2cnc(C(F)(F)F)cn2)CC1] | 620.2 | SFC analysis: 100% ee; retention time: 2.069 min; column: DAICEL CHIRALPAK ® MIC, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.41 (s, 1H), 8.30 (s, 1H), 7.99 (s, 1H), 7.23 (s, 1H), 7.11 (d, J = 8.4 Hz, 1H), 7.00 (d, J = 8.4 Hz, 1H), 6.63 (t, J = 56.4 Hz, 1H), 4.80-4.69 (m, 1H), 4.17 (dd, J = 13.3, 2.5 Hz, 1H), 3.98-3.66 (m, 8H), 3.56 (dd, J = 13.3, 7.6 Hz, 1H), 3.10-2.97 (m, 1H), 2.88-2.73 (m, 1H). |
| 146 | 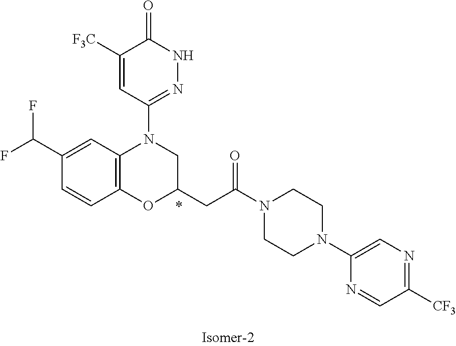 [SMILES: O=C(CC1CN(c2cc(C(F)(F)F)c(=O)[nH]n2)c2cc(C(F)F)ccc2O1)N1CCN(c2cnc(C(F)(F)F)cn2)CC1] | 620.2 | SFC analysis: 100% ee; retention time: 2.650 min; column: DAICEL CHIRALPAK ® MIC, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.41 (s, 1H), 8.30 (s, 1H), 7.99 (s, 1H), 7.23 (s, 1H), 7.12 (d, J = 8.4 Hz, 1H), 7.00 (d, J = 8.4 Hz, 1H), 6.63 (t, J = 56.4 Hz, 1H), 4.80-4.70 (m, 1H), 4.17 (dd, J = 13.2, 2.5 Hz, 1H), 3.93-3.65 (m, 8H), 3.56 (dd, J = 13.3, 7.6 Hz, 1H), 3.09-2.98 (m, 1H), 2.90-2.70 (m, 1H). |
| 147 | 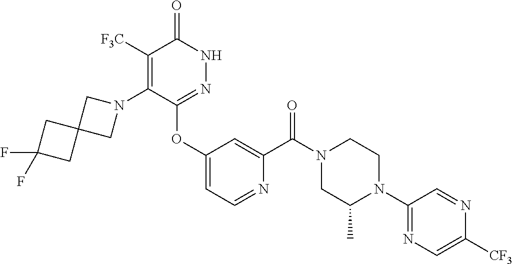 [SMILES: C[C@@H]1CN(C(=O)c2cc(Oc3n[nH]c(=O)c(C(F)(F)F)c3N3CC4(C3)CC(F)(F)C4)ccn2)CCN1c1cnc(C(F)(F)F)cn1] | 661.4 | 0.5TFA salt, ¹H NMR (400 MHz, DMSO-d₆, ppm): δ 11.90 (s, 1H), 8.75-8.51 (m, 2H), 8.41 (s, 1H), 7.69-7.53 (m, 1H), 7.50-7.35 (m, 1H), 4.93-4.17 (m, 7H), 4.05-3.68 (m, 1H), 3.67-3.45 (m, 1H), 3.33-3.15 (m, 2H), 3.00-2.75 (m, 4H), 1.22-1.08 (m, 3H). ¹⁹F NMR (376 MHz, DMSO-d₆, ppm): δ −51.54 (3F), −64.67 (3F), −91.05 (2F). |
| 148 | 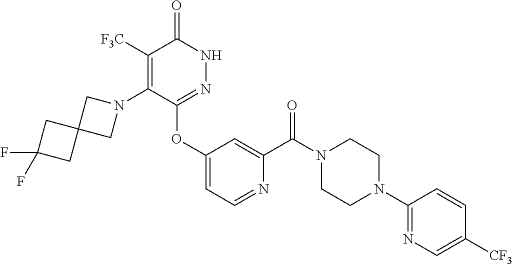 [SMILES: O=C(c1cc(Oc2n[nH]c(=O)c(C(F)(F)F)c2N2CC3(C2)CC(F)(F)C3)ccn1)N1CCN(c2ccc(C(F)(F)F)cn2)CC1] | 646.4 | 0.5TFA salt, ¹H NMR (400 MHz, DMSO-d₆, ppm): δ 11.92 (s, 1H), 8.66 (d, J = 5.6 Hz, 1H), 8.47 (s, 1H), 7.90 (d, J = 9.1 Hz, 1H), 7.58 (s, 1H), 7.45 (d, J = 5.6 Hz, 1H), 7.02 (d, J = 9.1 Hz, 1H), 4.54 (s, 4H), 3.90-3.80 (m, 4H), 3.79-3.69 (m, 2H), 3.68-3.58 (m, 2H), 2.99-2.81 (m, 4H). ¹⁹F NMR (376 MHz, DMSO-d₆, ppm): δ −51.55 (3F), −59.30 (3F), −91.05 (2F). |
| 149 | 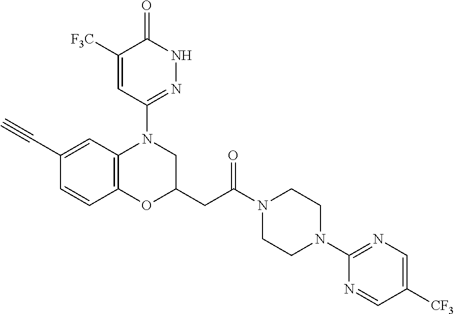 [SMILES: C#Cc1ccc2c(c1)N(c1cc(C(F)(F)F)c(=O)[nH]n1)CC(CC(=O)N1CCN(c3ncc(C(F)(F)F)cn3)CC1)O2] | 594.4 | ¹H NMR (400 MHz, DMSO-d₆, ppm): δ 13.10 (s, 1H), 8.75 (s, 2H), 8.01 (s, 1H), 7.24 (s, 1H), 7.00 (d, J = 8.3 Hz, 1H), 6.87 (d, J = 8.4 Hz, 1H), 4.70-4.60 (m, 1H), 4.10-4.00 (m, 2H), 3.99-3.76 (m, 4H), 3.69-3.55 (m, 4H), 3.54-3.45 (m, 1H), 3.00-2.90 (m, 1H), 2.85-2.72 (m, 1H). ¹⁹F NMR (376 MHz, DMSO-d₆, ppm): δ −59.28 (3F), −65.97 (3F). |
| 150 | 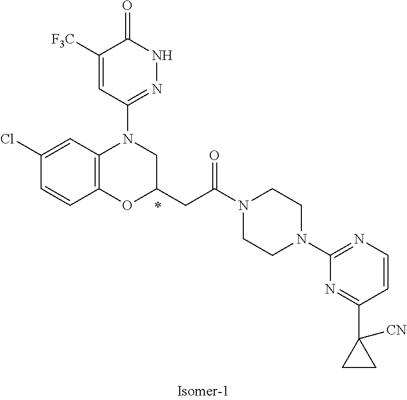 [SMILES: N#CC1(c2ccnc(N3CCN(C(=O)CC4CN(c5cc(C(F)(F)F)c(=O)[nH]n5)c5cc(Cl)ccc5O4)CC3)n2)CC1] | 601.3 | SFC analysis: 100% ee; retention time: 4.566 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.31 (d, J = 5.1 Hz, 1H), 7.95 (s, 1H), 7.06 (d, J = 1.9 Hz, 1H), 6.91-6.84 (m, 3H), 4.74-4.63 (m, 1H), 4.09 (dd, J = 13.2, 2.5 Hz, 1H), 3.92-3.74 (m, 4H), 3.72-3.60 (m, 4H), 3.53 (dd, J = 13.1, 7.4 Hz, 1H), 3.06- 2.94 (m, 1H), 2.84-2.72 (m, 1H), 1.85-1.78 (m, 2H), 1.77-1.70 (m, 2H). |
| 151 | 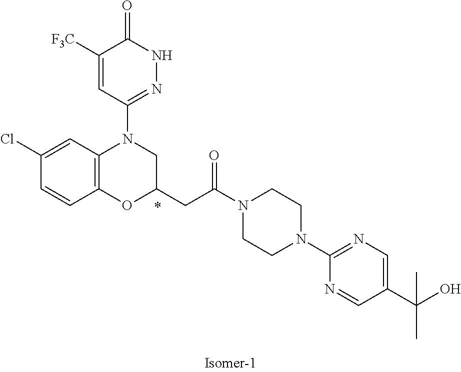 [SMILES: CC(C)(O)c1cnc(N2CCN(C(=O)CC3CN(c4cc(C(F)(F)F)c(=O)[nH]n4)c4cc(Cl)ccc4O3)CC2)nc1] | 576.3 [M − OH⁻]⁺ | SFC analysis: 100% ee; retention time; 4.379 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, DMSO-d₆, ppm): δ 8.45 (s, 2H), 8.01 (s, 1H), 7.22 (s, 1H), 6.93-6.84 (m, 2H), 5.09 (s, 1H), 4.68-4.55 (m, 1H), 4.05- 3.93 (m, 1H), 3.83-3.60 (m, 4H), 3.59-3.42 (m, 5H), 2.98- 2.86 (m, 1H), 2.81-2.70 (m, 1H), 1.41 (s, 6H). |
| 152 | 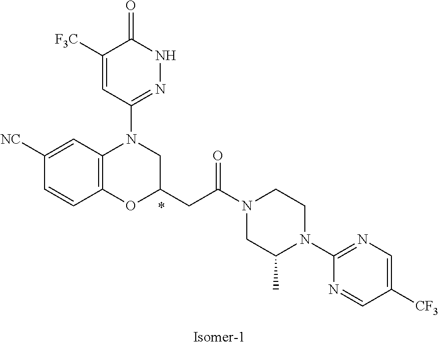 [SMILES: C[C@@H]1CN(C(=O)CC2CN(c3cc(C(F)(F)F)c(=O)[nH]n3)c3cc(C#N)ccc3O2)CCN1c1ncc(C(F)(F)F)cn1] | 609.2 | SFC analysis: 100% ee; retention time: 3.764 min; column: DAICEL CHIRALCEL ® AS, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 8.08-7.99 (m, 1H), 7.51 (d, J = 1.2 Hz, 1H), 7.26 (d, J = 8.4 Hz, 1H), 7.12-6.88 (m, 1H), 5.10-4.90 (m, 1H), 4.87-4.75 (m, 1H), 4.72-4.55 (m, 1H), 4.48-4.32 (m, 1H), 4.20-3.85 (m, 2H), 3.73-3.32 (m, 3H), 3.15-2.73 (m, 3H), 1.32-1.06 (m, 3H). |
| 153 | 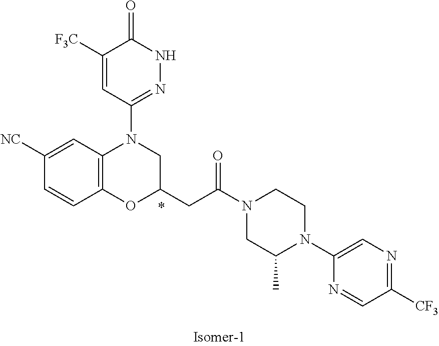 [SMILES: C[C@@H]1CN(C(=O)CC2CN(c3cc(C(F)(F)F)c(=O)[nH]n3)c3cc(C#N)ccc3O2)CCN1c1cnc(C(F)(F)F)cn1] | 609.2 | SFC analysis: 100% ee; retention time: 1.826 min; column: DAICEL CHIRALCEL ® AS, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.42 (s, 1H), 8.26 (s, 1H), 8.09-7.99 (m, 1H), 7.52 (s, 1H), 7.26 (d, J = 8.4 Hz, 1H), 7.01 (dd, J = 8.4, 5.2 Hz, 1H), 4.84-4.68 (m, 2H), 4.49-4.22 (m, 2H), 4.18-3.89 (m, 2H), 3.70-3.30 (m, 3H), 3.24-2.76 (m, 3H), 1.34-1.10 (m, 3H). |
| 154 | 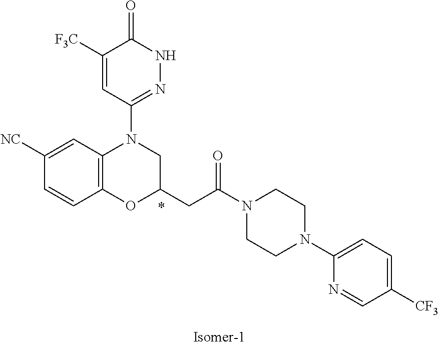 [SMILES: N#Cc1ccc2c(c1)N(c1cc(C(F)(F)F)c(=O)[nH]n1)CC(CC(=O)N1CCN(c3ccc(C(F)(F)F)cn3)CC1)O2] | 594.2 | SFC analysis: 100% ee; retention time: 4.630 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.37 (s, 1H), 7.99 (s, 1H), 7.75 (dd, J = 9.1, 2.2 Hz, 1H), 7.52 (d, J = 1.3 Hz, 1H), 7.35-7.22 (m, 1H), 7.02 (d, J = 8.4 Hz, 1H), 6.90 (d, J = 9.1 Hz, 1H), 4.82- 4.72 (m, 1H), 4.15 (dd, J = 13.3, 2.4 Hz, 1H), 3.83-3.64 (m, 8H), 3.56 (dd, J = 13.3, 7.6 Hz, 1H), 3.10-2.99 (m, 1H), 2.89-2.78 (m, 1H). |
| 155 | 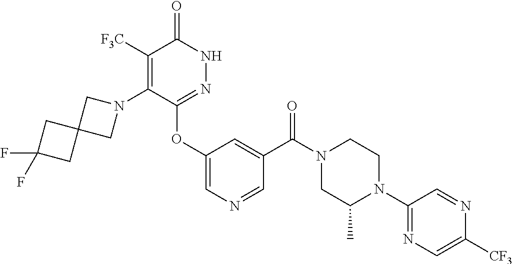 [SMILES: C[C@@H]1CN(C(=O)c2cncc(Oc3n[nH]c(=O)c(C(F)(F)F)c3N3CC4(C3)CC(F)(F)C4)c2)CCN1c1cnc(C(F)(F)F)cn1] | 661.2 | 0.8TFA salt, ¹H NMR (400 MHz, methanol-d₄, ppm): δ 8.64 (s, 1H), 8.58 (brs, 1H), 8.43 (s, 1H), 8.28 (s, 1H), 8.01-7.88 (m, 1H), 4.99-3.85 (m, 1H), 4.70 (s, 4H), 4.65-4.47 (m, 1H), 4.44-4.26 (m, 1H), 3.92-3.18 (m, 4H), 2.85 (t, J = 12.1 Hz, 4H), 1.40-1.10 (m, 3H). ¹⁹F NMR (376 MHz, methanol-d₄, ppm): δ −53.43 (3F), −67.90 (3F), −94.71 (2F). |
| 156 | 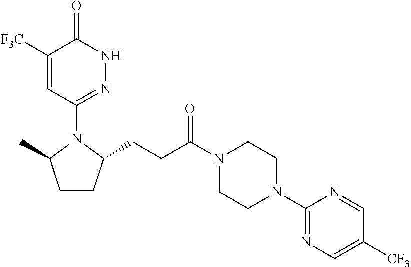 [SMILES: C[C@@H]1CC[C@@H](CCC(=O)N2CCN(c3ncc(C(F)(F)F)cn3)CC2)N1c1cc(C(F)(F)F)c(=O)[nH]n1] | 534.2 | SFC analysis: 100% ee; retention time: 3.197 min; column: DAICEL CHICEL ® OD, 250 × 4.6 mm, 5 μm, A for CO₂ and B for MeOH (0.1% DEA), 15% to 40%; pressure: 100 bar; flow rate: 2.5 mL/min. ¹H NMR (400 MHz, methanol-d₄, ppm): δ 8.59 (s, 2H), 7.84 (s, 1H), 3.99-3.87 (m, 6H), 3.74- 3.62 (m, 4H), 2.51 (t, J = 7.2 Hz, 2H), 2.17-1.97 (m, 3H), 1.91-1.67 (m, 3H), 1.29 (d, J = 6.2 Hz, 3H). |
| 157 | 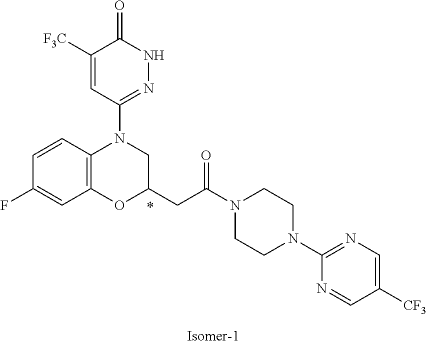 [SMILES: O=C(CC1CN(c2cc(C(F)(F)F)c(=O)[nH]n2)c2ccc(F)cc2O1)N1CCN(c2ncc(C(F)(F)F)cn2)CC1] | 588.2 | SFC analysis: 99.02% ee; retention time: 1.328 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 7.95 (s, 1H), 7.20-6.90 (m, 1H), 6.83- 6.54 (m, 2H), 4.70 (d, J = 6.0 Hz, 1H), 4.25-4.10 (m, 1H), 4.06-3.82 (m, 4H), 3.79-3.57 (m, 4H), 3.55-3.45 (m, 1H), 3.06-2.90 (m, 1H), 2.90-2.70 (m, 1H). |
| 158 | 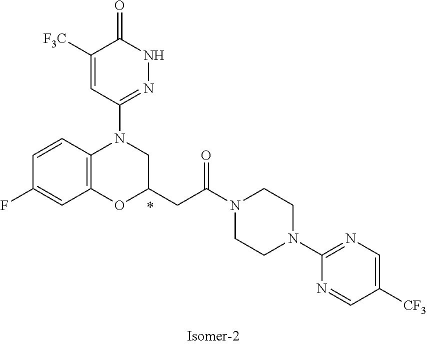 [SMILES: O=C(CC1CN(c2cc(C(F)(F)F)c(=O)[nH]n2)c2ccc(F)cc2O1)N1CCN(c2ncc(C(F)(F)F)cn2)CC1] | 588.2 | SFC analysis: 95.88% ee; retention time: 1.668 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 7.95 (s, 1H), 7.20-6.90 (m, 1H), 6.81- 6.32 (m, 2H), 4.70 (d, J = 6.0 Hz, 1H), 4.24-4.10 (m, 1H), 4.04-3.85 (m, 4H), 3.77-3.57 (m, 4H), 3.55-3.47 (m, 1H), 3.06-2.94 (m, 1H), 2.90-2.68 (m, 1H), |
| 159 | 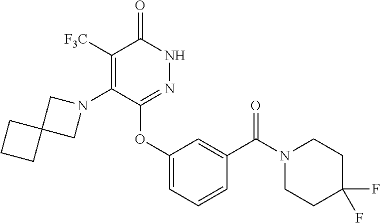 [SMILES: O=C(c1cccc(Oc2n[nH]c(=O)c(C(F)(F)F)c2N2CC3(CCC3)C2)c1)N1CCC(F)(F)CC1] | 499.2 | 0.3TFA salt, ¹H NMR (400 MHz, DMSO-d₆, ppm): δ 11.53 (s, 1H), 7.51-7.49 (m, 1H), 7.37-7.28 (m, 3H), 4.44 (s, 4H), 3.83-3.75 (m, 4H), 2.18-2.16 (m, 4H), 2.12-2.05 (m, 4H), 1.77-1.69 (m, 2H). ¹⁹F NMR (376 MHz, DMSO-d₆, ppm): δ −51.24 (3F), −95.68 (2F). |
| 160 | 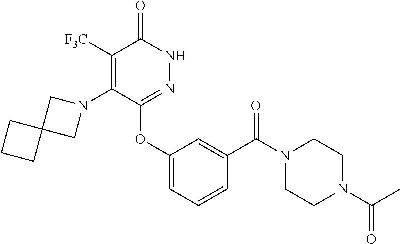 [SMILES: CC(=O)N1CCN(C(=O)c2cccc(Oc3n[nH]c(=O)c(C(F)(F)F)c3N3CC4(CCC4)C3)c2)CC1] | 506.4 | 0.5TFA salt, ¹H NMR (400 MHz, DMSO-d₆, ppm): δ 11.54 (s, 1H), 7.52-7.49 (m, 1H), 7.33-7.25 (m, 3H), 4.44 (s, 4H), 3.69-3.25 (m, 8H), 2.19-2.15 (m, 4H), 2.02 (s, 3H), 1.76- 1.71 (m, 2H). ¹⁹F NMR (376 MHz, DMSO-d₆, ppm): δ −51.24 (3F). |
| 161 | 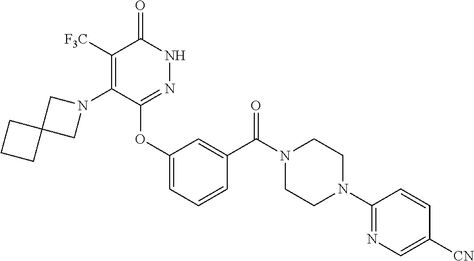 [SMILES: N#Cc1ccc(N2CCN(C(=O)c3cccc(Oc4n[nH]c(=O)c(C(F)(F)F)c4N4CC5(CCC5)C4)c3)CC2)nc1] | 566.4 | 1.5TFA salt, ¹H NMR (400 MHz, DMSO-d₆, ppm): δ 11.55 (s, 1H), 8.52 (s, 1H), 7.92-7.89 (m, 1H), 7.53-7.51 (m, 1H), 7.38-7.28 (m, 3H), 6.95-6.90 (m, 1H), 4.44 (s, 4H), 3.78- 3.45 (m, 8H), 2.19-2.15 (m, 4H), 1.77-1.70 (m, 2H). ¹⁹F NMR (376 MHz, DMSO-d₆, ppm): δ −51.24 (3F). |
| 162 | 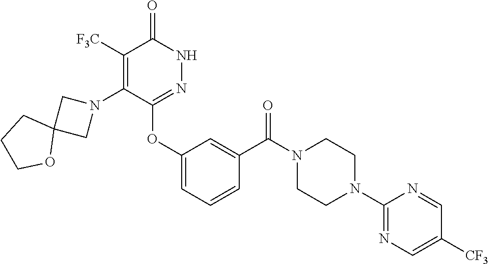 [SMILES: O=C(c1cccc(Oc2n[nH]c(=O)c(C(F)(F)F)c2N2CC3(CCCO3)C2)c1)N1CCN(c2ncc(C(F)(F)F)cn2)CC1] | 626.5 | 0.3TFA salt, ¹H NMR (400 MHz, DMSO-d₆, ppm): δ 11.64 (s, 1H), 8.75 (s, 2H), 7.54-7.50 (m, 1H), 7.40-7.37 (m, 1H), 7.36-7.29 (m, 2H), 4.50-4.47 (m, 2H), 4.41-4.38 (m, 2H), 3.79-3.88 (m, 4H), 3.77-3.70 (m, 4H), 3.65-3.55 (m, 2H), 2.10-2.06 (m, 2H), 1.88-1.75 (m, 2H). ¹⁹F NMR (376 MHz, DMSO-d₆, ppm): δ −51.26 (3F), −59.30 (3F). |
| 163 | 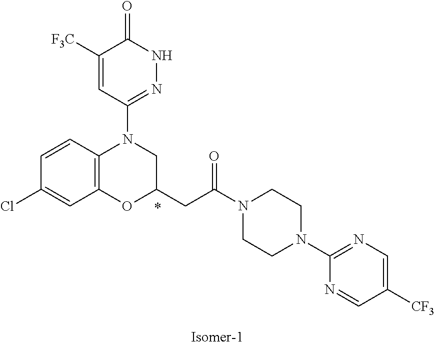 [SMILES: O=C(CC1CN(c2cc(C(F)(F)F)c(=O)[nH]n2)c2ccc(Cl)cc2O1)N1CCN(c2ncc(C(F)(F)F)cn2)CC1] | 604.0 | SFC analysis: 100% ee; retention time: 3.817 min; column: DAICEL CHIRALCEL ® AS, 100 × 3.0 mm, 3 μm, A for CO₂ and B for MeOH (0.1% DEA), 5% to 40%; pressure: 100 bar, flow rate: 1.5 mL/min. ¹H NMR (400 MHz, methanol-d₄, ppm): δ 8.60 (s, 2H), 7.96 (s, 1H), 7.01 (d, J = 8.7 Hz, 1H), 6.92 (d, J = 2.3 Hz, 1H), 6.85 (dd, J = 8.7, 2,.3 Hz, 1H), 4.76-4.65 (m, 1H), 4.20-4.09 (m, 1H), 4.06-3.86 (m, 4H), 3.79-3.61 (m, 4H), 3.57-3.47 (m, 1H), 3.06-2.96 (m, 1H), 2.86-2.77 (m, 1H). |
| 164 | 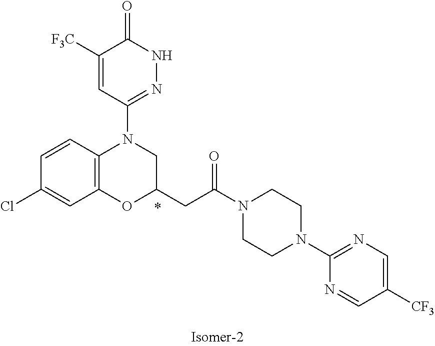 [SMILES: O=C(CC1CN(c2cc(C(F)(F)F)c(=O)[nH]n2)c2ccc(Cl)cc2O1)N1CCN(c2ncc(C(F)(F)F)cn2)CC1] | 604.0 | SFC analysis: 100% ee; retention time: 3.485 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.59 (s, 2H), 7.96 (s, 1H), 7.01 (d, J = 8.7 Hz, 1H), 6.92 (s, 1H), 6.85 (dd, J = 8.7, 1.5 Hz, 1H), 4.75-4.66 (m, 1H), 4.19-4.09 (m, 1H), 4.08-3.85 (m, 4H), 3.80-3.59 (m, 4H), 3.58-3.47 (m, 1H), 3.07-2.96 (m, 1H), 2.85-2.75 (m, 1H). |
| 165 | 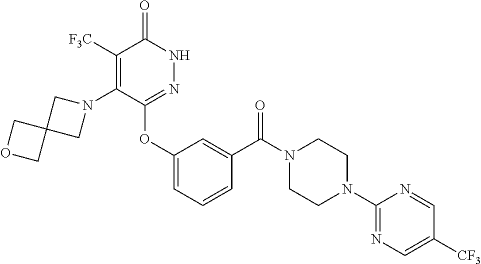 [SMILES: O=C(c1cccc(Oc2n[nH]c(=O)c(C(F)(F)F)c2N2CC3(COC3)C2)c1)N1CCN(c2ncc(C(F)(F)F)cn2)CC1] | 611.9 | ¹H NMR (300 MHz, DMSO-d₆, ppm): δ 11.60 (brs, 1H), 8.74 (s, 2H), 7.53 (t, J = 7.8 Hz, 1H), 7.34-7.29 (m, 3H), 4.67 (s, 4H), 4.62 (s, 4H), 3.94-3.92 (m, 4H), 3.72-3.47 (m, 4H). ¹⁹F NMR (282 MHz, DMSO-d₆, ppm): δ −51.89 (3F), −60.20 (3F). |
| 166 | 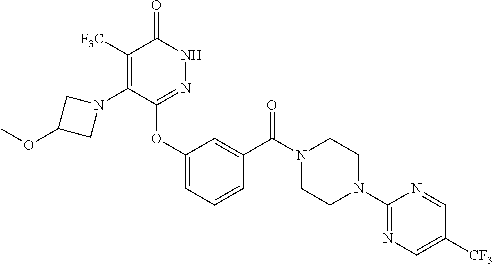 [SMILES: COC1CN(c2c(Oc3cccc(C(=O)N4CCN(c5ncc(C(F)(F)F)cn5)CC4)c3)n[nH]c(=O)c2C(F)(F)F)C1] | 600.0 | ¹H NMR (400 MHz, DMSO-d₆, ppm): δ 11.61 (s, 1H), 8.74 (s, 2H), 7.52 (t, J = 8.0 Hz, 1H), 7.37-7.28 (m, 3H), 4.67- 4.60 (m, 2H), 4.29-4.18 (m, 3H), 3.95-3.91 (m, 4H), 3.80- 3.60 (m, 2H), 3.50-3.40 (m, 2H), 3.23 (s, 3H). ¹⁹F NMR (376 MHz, DMSO-d₆, ppm): δ −51.30 (3F), −59.31 (3F). |
| 167 | 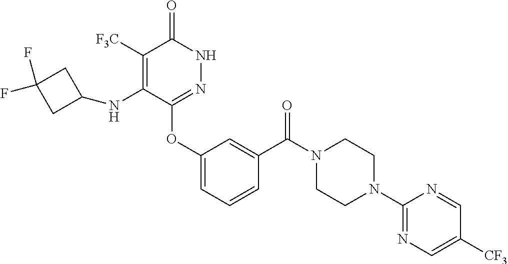 [SMILES: O=C(c1cccc(Oc2n[nH]c(=O)c(C(F)(F)F)c2NC2CC(F)(F)C2)c1)N1CCN(c2ncc(C(F)(F)F)cn2)CC1] | 620.0 | 0.3TFA salt, ¹H NMR (400 MHz, DMSO-d₆, ppm): δ 11.77 (s, 1H), 8.74 (s, 2H), 7.55-7.46 (m, 2H), 7.39-7.31 (m, 3H), 4.02-3.85 (m, 5H), 3.80-3.30 (m, 2H), 3.46 (s, 2H), 3.03- 2.89 (m, 4H). ¹⁹F NMR (376 MHz, DMSO-d₆, ppm): δ −55.44 (3F), −59.40 (3F), −83.77 (1F), −98.33 (1F). |
| 168 | 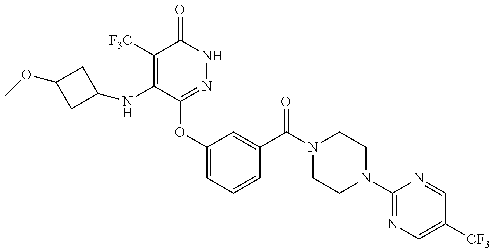 [SMILES: COC1CC(Nc2c(Oc3cccc(C(=O)N4CCN(c5ncc(C(F)(F)F)cn5)CC4)c3)n[nH]c(=O)c2C(F)(F)F)C1] | 614.0 | 0.2TFA salt, ¹H NMR (400 MHz, DMSO-d₆, ppm): δ 11.67 (s, 1H), 8.73 (s, 2H), 7.54-7.50 (m, 1H), 7.37-7.24 (m, 4H), 3.94-3.88 (m, 4H), 3.80-3.40 (m, 6H), 3.12 (s, 3H), 2.67- 2.66 (m, 2H), 2.17-2.10 (m, 2H). ¹⁹F NMR (376 MHz, DMSO-d₆, ppm): δ −55.32 (3F), −59.39 (3F). |
| 169 | 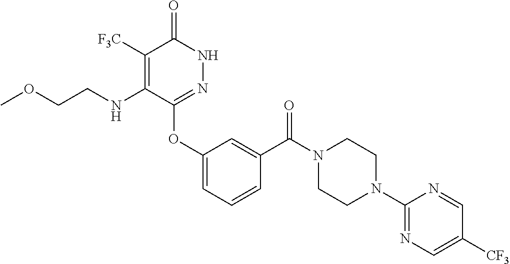 [SMILES: COCCNc1c(Oc2cccc(C(=O)N3CCN(c4ncc(C(F)(F)F)cn4)CC3)c2)n[nH]c(=O)c1C(F)(F)F] | 588.0 | 0.3TFA salt, ¹H NMR (400 MHz, DMSO-d₆, ppm): δ 11.74 (s, 1H), 8.74 (s, 2H), 7.53 (t, J = 8.0 Hz, 1H), 7.35-7.30 (m, 3H), 6.82 (s, 1H), 3.93-3.90 (m, 4H), 3.74-3.70 (m, 2H), 3.56-3.53 (m, 6H), 3.25-3.20 (m, 3H). ¹⁹F NMR (376 MHz, DMSO-d₆, ppm): δ −54.74 (3F), −59.40 (3F). |
| 170 | 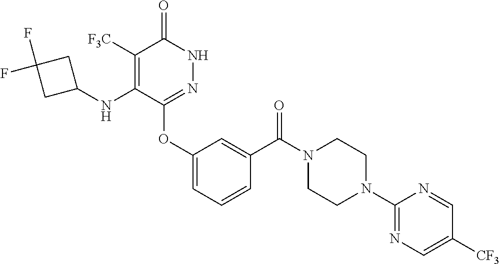 [SMILES: O=C(c1cccc(Oc2n[nH]c(=O)c(C(F)(F)F)c2NC2CC(F)(F)C2)c1)N1CCN(c2ncc(C(F)(F)F)cn2)CC1] | 621.4 | ¹H NMR (400 MHz, DMSO-d₆, ppm): δ 12.61 (s, 1H), 8.75 (s, 2H), 7.55 (t, J = 7.8 Hz, 1H), 7.46-7.37 (m, 2H), 7.34 (d, J = 7.6 Hz, 1H), 5.32-5.26 (m, 1H), 3.99-3.84 (m, 4H), 3.80- 3.64 (m, 2H), 3.53-3.38 (m, 2H), 3.19-3.08 (m, 2H), 2.97- 2.85 (m, 2H). |
| 171 | 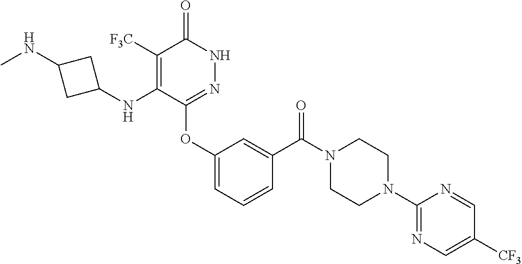 [SMILES: CNC1CC(Nc2c(Oc3cccc(C(=O)N4CCN(c5ncc(C(F)(F)F)cn5)CC4)c3)n[nH]c(=O)c2C(F)(F)F)C1] | 613.6 | 1.4TFA salt, ¹H NMR (400 MHz, DMSO-d₆, ppm): δ 11.79- 11.72 (m, 1H), 8.75 (s, 2H), 8.69-8.55 (m, 2H), 7.58-7.48 (m, 2H), 7.42-7.30 (m, 3H), 4.30-4.26 (m, 1H), 4.00-3.84 (m, 5H), 3.78-3.62 (m, 4H), 3.60-3.40 (m, 3H), 2.60-2.45 (m, 4H). ¹⁹F NMR (376 MHz, DMSO-d₆, ppm): δ −55.25 (3F), −59.28 (3F). |
| 172 | 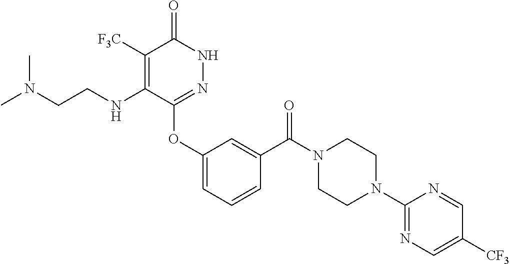 [SMILES: CN(C)CCNc1c(Oc2cccc(C(=O)N3CCN(c4ncc(C(F)(F)F)cn4)CC3)c2)n[nH]c(=O)c1C(F)(F)F] | 601.6 | 1.7TFA salt, ¹H NMR (400 MHz, DMSO-d₆, ppm): δ 11.83 (s, 1H), 9.9-9.45 (m, 1H), 8.75 (s, 2H), 7.58-7.53 (m, 1H), 7.42-7.33 (m, 3H), 7.25-7.18 (m, 1H), 3.94-3.90 (m, 4H), 3.83-3.76 (m, 4H), 3.45-3.39 (m, 4H), 2.82 (s, 6H). ¹⁹F NMR (376 MHz, DMSO-d₆, ppm): δ −55.02 (3F), −59.28 (3F). |
| 173 | 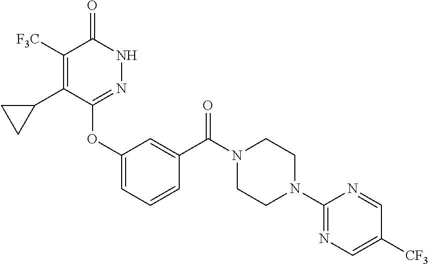 [SMILES: O=C(c1cccc(Oc2n[nH]c(=O)c(C(F)(F)F)c2C2CC2)c1)N1CCN(c2ncc(C(F)(F)F)cn2)CC1] | 555.4 | ¹H NMR (400 MHz, DMSO-d₆, ppm): δ 12.77 (s, 1H), 8.74 (s, 2H), 7.54-7.50 (m, 1H), 7.34- 7.28 (m, 3H), 3.94-3.89 (m, 4H), 3.75-3.68 (m, 2H), 3.49- 3.41 (m, 2H), 1.96-1.92 (m, 1H), 1.08-1.05 (m, 2H), 0.93- 0.91 (m, 2H). ¹⁹F NMR (376 MHz, DMSO-d₆, ppm): δ −58.10 (3F), −59.30 (3F). |
| 174 | 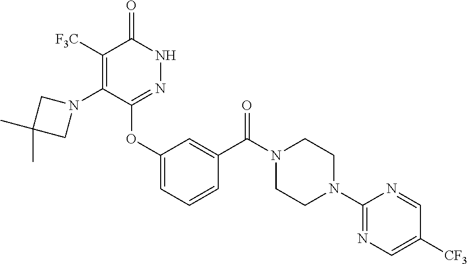 [SMILES: CC1(C)CN(c2c(Oc3cccc(C(=O)N4CCN(c5ncc(C(F)(F)F)cn5)CC4)c3)n[nH]c(=O)c2C(F)(F)F)C1] | 598.4 | 0.23TFA salt, ¹H NMR (400 MHz, DMSO-d₆, ppm): δ 11.59 (s, 1H), 8.74 (s, 2H), 7.51 (t, J = 8 Hz, 1H), 7.39-7.22 (m, 3H), 4.14 (s, 4H), 4.00-3.84 (m, 4H), 3.76-3.65 (m, 2H), 3.50-3.42 (m, 2H), 1.25 (s, 6H). ¹⁹F NMR (376 MHz, DMSO-d₆, ppm): δ −51.23 (3F), −59.29 (3F). |
| 176 | 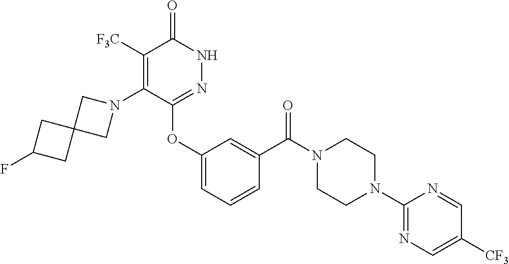 [SMILES: O=C(c1cccc(Oc2n[nH]c(=O)c(C(F)(F)F)c2N2CC3(CC(F)C3)C2)c1)N1CCN(c2ncc(C(F)(F)F)cn2)CC1] | 628.4 | 0.39TFA salt, ¹H NMR (400 MHz, DMSO-d₆, ppm) δ 11.58 (s, 1H), 8.74 (s, 2H), 7.52 (t, J = 8.0 Hz, 1H), 7.37-7.29 (m, 3H), 5.07-4.86 (m, 1H), 4.47 (s, 4H), 3.95-3.89 (m, 4H), 3.72-3.66 (m, 2H), 3.55-3.50 (m, 2H), 2.67-2.60 (m, 2H), 2.42-2.31 (m, 2H). ¹⁹F NMR (376 MHz, DMSO-d₆, ppm): δ −51.34 (3F), −59.29 (3F), −167.98 (1F). |
| 177 | 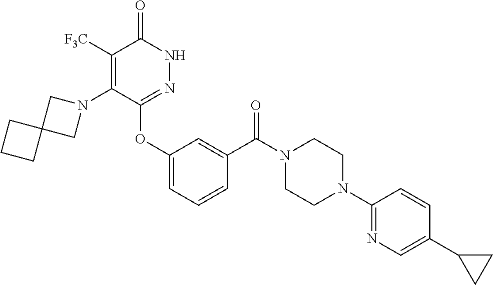 [SMILES: O=C(c1cccc(Oc2n[nH]c(=O)c(C(F)(F)F)c2N2CC3(CCC3)C2)c1)N1CCN(c2ccc(C3CC3)cn2)CC1] | 581.4 | 0.2FA salt, ¹H NMR (400 MHz, DMSO-d₆, ppm): δ 11.54 (s, 1H), 8.30 (brs, 1H), 7.98 (s, 1H), 7.53-7.49 (t, J = 8.0 Hz, 1H), 7.31-7.27 (m, 2H), 7.26-7.23 (m, 2H), 6.77 (d, J = 8.0 Hz, 1H), 4.44 (s, 4H), 3.87-3.25 (m, 8H), 2.22-2.15 (m, 4H), 1.87-1.66 (m, 3H), 0.91-0.84 (m, 2H), 0.65-0.56 (m, 2H). |
| 178 | 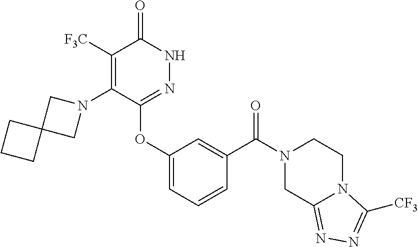 [SMILES: O=C(c1cccc(Oc2n[nH]c(=O)c(C(F)(F)F)c2N2CC3(CCC3)C2)c1)N1CCn2c(nnc2C(F)(F)F)C1] | 570.2 | 2.0TFA salt, ¹H NMR (400 MHz, DMSO-d₆, ppm): δ 11.55 (s, 1H), 7.58-7.53 (m, 1H), 7.47-7.43 (m, 1H), 7.40-7.36 (m, 2H), 5.10-4.85 (m, 2H), 4.44 (s, 4H), 4.25-4.24 (m, 2H), 3.82-3.78 (m, 2H), 2.21-2.15 (m, 4H), 1.79-1.68 (m, 2H). ¹⁹F NMR (376 MHz, DMSO-d₆, ppm): δ −51.59 (3F), −61.81 (3F). |
| 179 | 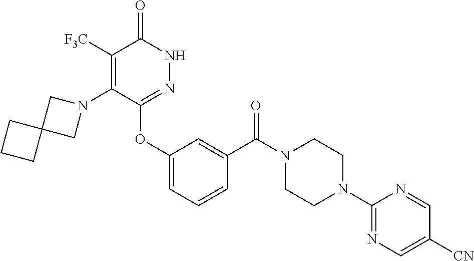 [SMILES: N#Cc1cnc(N2CCN(C(=O)c3cccc(Oc4n[nH]c(=O)c(C(F)(F)F)c4N4CC5(CCC5)C4)c3)CC2)nc1] | 567.4 | 1.1TFA salt, ¹H NMR (400 MHz, DMSO-d₆, ppm): δ 11.57 (s, 1H), 8.80 (s, 2H), 7.57-7.50 (m, 1H), 7.40-7.29 (m, 3H), 4.44 (s, 4H), 4.02-3.81 (m, 4H), 3.79-3.71 (m, 4H), 2.19- 2.15 (m, 4H), 1.77-1.69 (m, 2H). ¹⁹F NMR (376 MHz, DMSO-d₆, ppm): δ −51.24 (3F). |
| 180 | 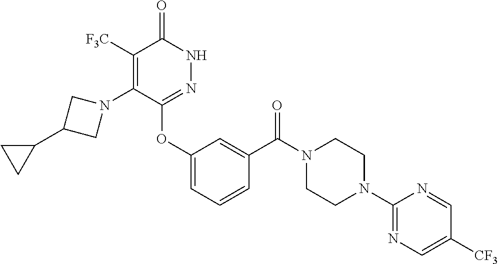 [SMILES: O=C(c1cccc(Oc2n[nH]c(=O)c(C(F)(F)F)c2N2CC(C3CC3)C2)c1)N1CCN(c2ncc(C(F)(F)F)cn2)CC1] | 610.5 | ¹H NMR (400 MHz, DMSO-d₆, ppm): δ 11.45 (s, 1H), 8.60 (s, 2H), 7.40-7.36 (m, 1H), 7.22-7.15 (m, 3H), 4.41-4.36 (m, 2H), 4.02-3.98 (m, 2H), 3.79- 3.75 (m, 4H), 3.60-3.52 (m, 2H), 3.32-3.30 (m, 2H), 2.12- 2.09 (m, 1H), 1.10-0.94 (m, 1H), 0.33-0.30 (m, 2H), 0.30- 0.28 (m, 2H). ¹⁹F NMR (376 MHz, DMSO-d₆, ppm): δ −51.22 (3F), −59.30 (3F). |
| 181 | 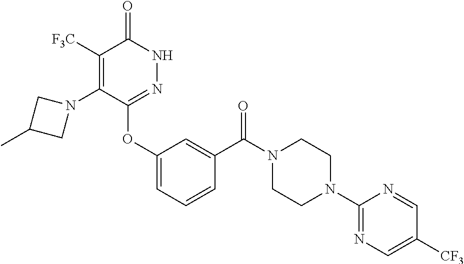 [SMILES: CC1CN(c2c(Oc3cccc(C(=O)N4CCN(c5ncc(C(F)(F)F)cn5)CC4)c3)n[nH]c(=O)c2C(F)(F)F)C1] | 584.2 | 1.0TFA salt, ¹H NMR (400 MHz, DMSO-d₆, ppm): δ 11.57 (s, 1H), 8.75 (s, 2H), 7.53-7.50 (m, 1H), 7.36-7.29 (m, 3H), 4.60-4.55 (m, 2H), 4.05-4.02 (m, 2H), 3.93-3.90 (m, 4H), 3.73-3.69 (m, 2H), 3.52-3.30 (m, 2H), 2.78-2.71 (m, 1H), 1.21-1.19 (m, 3H). ¹⁹F NMR (376 MHz, DMSO-d₆, ppm): δ −51.21 (3F), −59.29 (3F). |
| 182 | 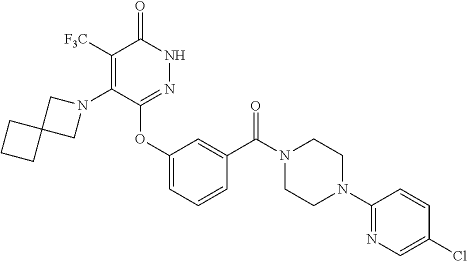 [SMILES: O=C(c1cccc(Oc2n[nH]c(=O)c(C(F)(F)F)c2N2CC3(CCC3)C2)c1)N1CCN(c2ccc(Cl)cn2)CC1] | 575.4 | 1.0TFA salt, ¹H NMR (400 MHz, DMSO-d₆, ppm): δ 11.55 (s, 1H), 8.14-8.13 (m, 1H), 7.66-7.64 (m, 1H), 7.53-7.50 (m, 1H), 7.36-7.28 (m, 4H), 4.44 (s, 4H), 3.59-3.34 (m, 8H), 2.19-2.15 (m, 4H), 1.75-1.71 (m, 2H). ¹⁹F NMR (376 MHz, DMSO-d₆, ppm): δ −51.24 (3F). |
| 184 | 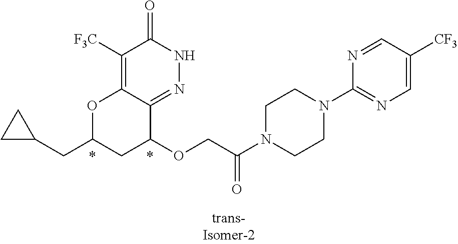 [SMILES: O=C(COC1CC(CC2CC2)Oc2c1n[nH]c(=O)c2C(F)(F)F)N1CCN(c2ncc(C(F)(F)F)cn2)CC1] | 563.4 | SFC analysis: 100% ee; retention time: 1.068 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 13.25 (s, 1H), 8.75 (s, 2H), 4.70-4.62 (m, 1H), 4.50 (t, J = 2.8 Hz, 1H), 4.38 (s, 2H), 3.96-3.68 (m, 4H), 3.60-3.39 (m, 4H), 2.41-2.37 (m, 1H), 2.10-1.96 (m, 1H), 1.77-1.69 (m, 1H), 1.55-1.49 (m, 1H), 0.88-0.85 (m, 1H), 0.50-0.42 (m, |
| | | | 2H), 0.20-0.09 (m, 2H). |
| | | | ¹⁹F NMR (376 MHz, DMSO-d₆, |
| | | | ppm): δ −57.37 (3F), −59.30 (3F). |
| 185 | 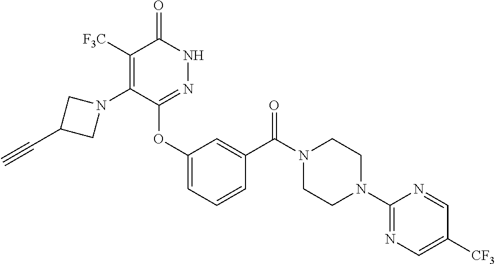 [SMILES: C#CC1CN(c2c(Oc3cccc(C(=O)N4CCN(c5ncc(C(F)(F)F)cn5)CC4)c3)n[nH]c(=O)c2C(F)(F)F)C1] | 594.4 | ¹H NMR (400 MHz, DMSO-d₆, ppm): δ 11.69 (s, 1H), 8.74 (s, 2H), 7.52 (t, J = 8.0 Hz, 1H), 7.39-7.27 (m, 3H), 4.78- 4.65 (m, 2H), 4.39-4.30 (m, 2H), 3.95-3.86 (m, 4H), 3.72- 3.65 (m, 2H), 3.56-3.52 (m, 1H), 3.46-3.41 (m, 2H), 3.34- 3.31 (m, 1H). ¹⁹F NMR (376 MHz, DMSO-d₆, ppm): δ −51.56 (3F), −59.29 (3F). |
| 186 | 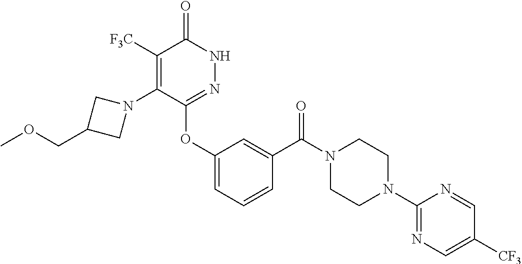 [SMILES: COCC1CN(c2c(Oc3cccc(C(=O)N4CCN(c5ncc(C(F)(F)F)cn5)CC4)c3)n[nH]c(=O)c2C(F)(F)F)C1] | 614.2 | ¹H NMR (400 MHz, DMSO-d₆, ppm): δ 11.60 (s, 1H), 8.74 (s, 2H), 7.52 (t, J = 8.0 Hz, 1H), 7.37-7.24 (m, 3H), 4.50 (t, J = 9.2 Hz, 2H), 4.19-4.15 (m, 2H), 3.97-3.86 (m, 4H), 3.72-3.68 (m, 2H), 3.51-3.42 (m, 4H), 3.26 (s, 3H), 2.92-2.79 (m, 1H). ¹⁹F NMR (376 MHz, DMSO-d₆, ppm): δ −51.16 (3F), −59.29 (3F). |
| 187 | 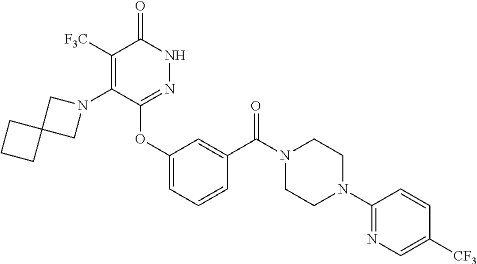 [SMILES: O=C(c1cccc(Oc2n[nH]c(=O)c(C(F)(F)F)c2N2CC3(CCC3)C2)c1)N1CCN(c2ccc(C(F)(F)F)cn2)CC1] | 609.2 | ¹H NMR (400 MHz, DMSO-d₆, ppm): δ 11.55 (s, 1H), 8.43 (s, 1H), 7.85 (dd, J = 9.2, 1.6 Hz, 1H), 7.52 (t, J = 8.0 Hz, 1H), 7.39-7.20 (m, 3H), 6.97 (d, J = 9.2 Hz, 1H), 4.43 (s, 4H), 3.76-3.68 (m, 6H), 3.49-3.45 (m, 2H), 2.17 (t, J 32 7.6 Hz, 4H), 1.73 (t, J = 7.6 Hz, 1H), ¹⁹F NMR (376 MHz, DMSO-d₆, ppm): δ −51.24 (3F), −59.30 (3F). |
| 188 | 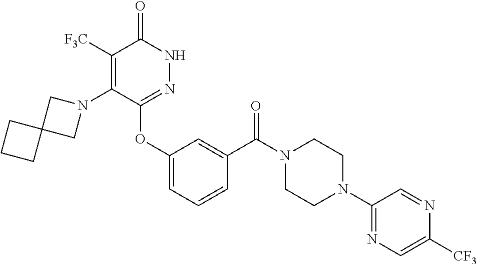 [SMILES: O=C(c1cccc(Oc2n[nH]c(=O)c(C(F)(F)F)c2N2CC3(CCC3)C2)c1)N1CCN(c2cnc(C(F)(F)F)cn2)CC1] | 610.2 | ¹H NMR (400 MHz, DMSO-d₆, ppm): δ 11.54 (s, 1H), 8.52 (s, 1H), 8.44 (s, 1H), 7.52 (t, J = 8.0 Hz, 1H), 7.41-7.26 (m, 3H), 4.44 (s, 4H), 3.82-3.70 (m, 6H), 3.50-3.43 (m, 2H), 2.17 (t, J = 7.6 Hz, 4H), 1.77-1.66 (m, 2H). ¹⁹F NMR (376 MHz, DMSO-d₆, ppm): δ −51.24 (3F), −64.67 (3F). |
| 189 | 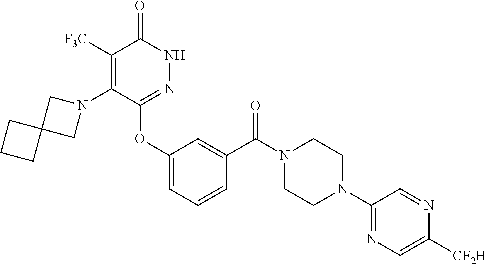 [SMILES: O=C(c1cccc(Oc2n[nH]c(=O)c(C(F)(F)F)c2N2CC3(CCC3)C2)c1)N1CCN(c2cnc(C(F)F)cn2)CC1] | 592.2 | ¹H NMR (400 MHz, DMSO-d₆, ppm): δ 11.56 (s, 1H), 8.59 (s, 2H), 7.52 (t, J = 8.0 Hz, 1H), 7.38-7.26 (m, 3H), 6.98 (t, J = 56.0 Hz, 1H), 4.44 (s, 4H), 3.90-3.82 (m, 4H), 3.72-3.67 (m, 2H), 3.48-3.43 (m, 2H), 2.17 (t, J = 7.6 Hz, 4H), 1.85- 1.68 (m, 2H). ¹⁹F NMR (376 MHz, DMSO-d₆, ppm): δ −51.24 (3F), −109.04 (2F). |
| 190 | 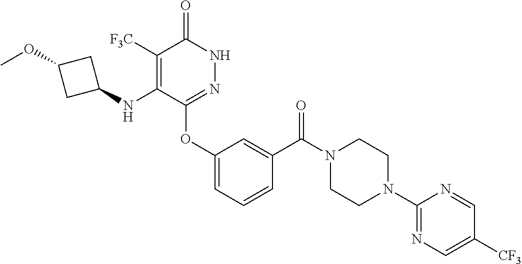 [SMILES: CO[C@H]1C[C@H](Nc2c(Oc3cccc(C(=O)N4CCN(c5ncc(C(F)(F)F)cn5)CC4)c3)n[nH]c(=O)c2C(F)(F)F)C1] | 614.0 | ¹H NMR (400 MHz, methanol-d₄, ppm): δ 8.60 (s, 1H), 7.58-7.53 (m, 1H), 7.37-7.34 (m, 3H), 4.42-4.38 (m, 1H), 4.04-3.95 (m, 5H), 3.86-3.82 (m, 2H), 3.59-3.54 (m, 2H), 3.24 (s, 3H), 2.51-2.45 (m, 4H). ¹⁹F NMR (376 MHz, methanol-d₄, ppm): δ −55.14 (3F), −59.30 (3F). |
| 191 | 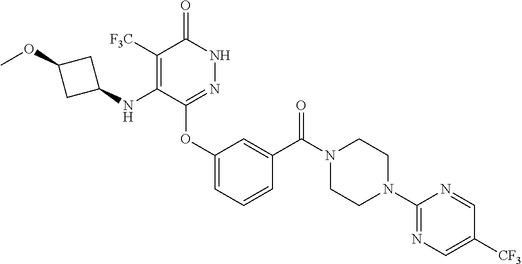 [SMILES: CO[C@H]1C[C@@H](Nc2c(Oc3cccc(C(=O)N4CCN(c5ncc(C(F)(F)F)cn5)CC4)c3)n[nH]c(=O)c2C(F)(F)F)C1] | 614.0 | ¹H NMR (400 MHz, methanol-d₄, ppm): δ 8.60 (s, 1H), 7.58-7.53 (m, 1H), 7.37-7.34 (m, 3H), 4.05-3.90 (m, 4H), 3.88-3.83 (m, 3H), 3.69-3.52 (m, 3H), 3.25 (s, 3H), 2.86- 2.74 (m, 2H), 2.14-2.03 (m, 2H). ¹⁹F NMR (376 MHz, DMSO-d₆, ppm): δ −55.27 (3F), −59.31 (3F). |
| 192 | 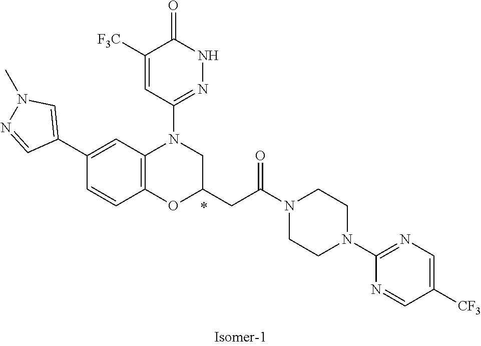 [SMILES: Cn1cc(-c2ccc3c(c2)N(c2cc(C(F)(F)F)c(=O)[nH]n2)CC(CC(=O)N2CCN(c4ncc(C(F)(F)F)cn4)CC2)O3)cn1] | 650.4 | SFC analysis: 100% ee; retention time: 3.345 min; column: Regis WHELK-O1(S, S), 50 × 4.6 mm, 3 μm, A for CO₂and B for MeOH (0.1% DEA), 40%; pressure: 100 bar; flow rate: 1.5 mL/min. ¹H NMR (400 MHz, DMSO-d₆, ppm): δ 13.08 (s, 1H), 8.74 s, 2H), 8.01- 7.97 (m, 2H), 7.71 (d, J = 0.4 Hz, 1H), 7.26 (d, J = 2.0 Hz, 1H), 7.10-7.07 (m, 1H), 6.85 (d, J = 8.4 Hz, 1H), 4.65-4.60 (m, 1H), 4.10-4.00 (m, 1H), 3.90-3.80 (m, 7H), 3.64-3.56 (m, 4H), 3.55-3.45 (m, 1H), 2.95-2.85 (m, 1H), 2.82-2.70 (m, 1H). ¹⁹F NMR (376 MHz, DMSO-d₆, ppm): δ −59.29 (3F), −65.99 (3F). |
| 193 | 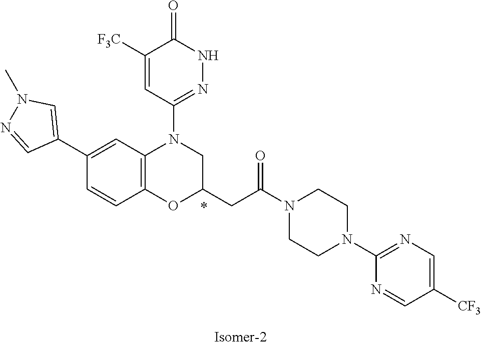 [SMILES: Cn1cc(-c2ccc3c(c2)N(c2cc(C(F)(F)F)c(=O)[nH]n2)CC(CC(=O)N2CCN(c4ncc(C(F)(F)F)cn4)CC2)O3)cn1] | 650.4 | SFC analysis: 98.44% ee; retention time: 4.911 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 13.08 (s, 1H), 8.74 (s, 2H), 8.01-7.97 (m, 2H), 7.71 (s, 1H), 7.26 (d, J = 2.0 Hz, 1H), 7.10-7.07 (m, 1H), 6.85 (d, J = 8.4 Hz, 1H), 4.65-4.60 (m, 1H), 4.10-4.00 (m, 1H), 3.92-3.80 (m, 7H), 3.64-3.56 (m, 4H), 3.53-3.45 (m, 1H), 2.95-2.84 (m, 1H), 2.82-2.70 (m, 1H). ¹⁹F NMR (376 MHz, DMSO-d₆, ppm): δ −59.29 (3F), −65.99 (3F). |
| 194 | 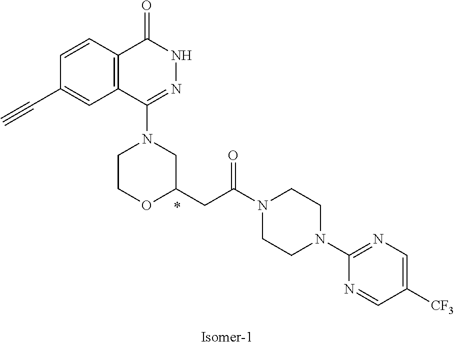 [SMILES: C#Cc1ccc2c(=O)[nH]nc(N3CCOC(CC(=O)N4CCN(c5ncc(C(F)(F)F)cn5)CC4)C3)c2c1] | 528.3 | ¹H NMR (400 MHz, methanol-d₄, ppm): δ 8.59 (s, 2H), 8.37 (d, J = 1.3 Hz, 1H), 8.05 (d, J = 8.4 Hz, 1H), 7.95 (dd, J = 8.4, 1.6 Hz, 1H), 4.31-4.20 (m, 1H), 4.08-3.83 (m, 7H), 3.81-3.59 (m, 4H), 3.52 (d, J = 12.3 Hz, 1H), 3.36-3.30 (m, 1H), 3.06-2.94 (m, 1H), 2.87-2.72 (m, 2H), 2.63-2.53 (m, 1H). |
| 195 | 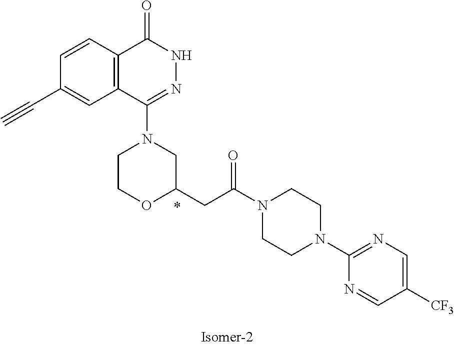 [SMILES: C#Cc1ccc2c(=O)[nH]nc(N3CCOC(CC(=O)N4CCN(c5ncc(C(F)(F)F)cn5)CC4)C3)c2c1] | 528.3 | ¹H NMR (400 MHz, methanol-d₄, ppm): δ 8.59 (s, 2H), 8.37 (d, J = 1.5 Hz, 1H), 8.05 (d, J = 8.4 Hz, 1H), 7.96 (dd, J = 8.4, 1.7 Hz, 1H), 4.30-4.20 (m, 1H), 4.09-3.81 (m, 7H), 3.80-3.60 (m, 4H), 3.52 (d, J = 12.3 Hz, 1H), 3.36-3.30 (m, 1H), 3.05-2.96 (m, 1H), 2.8-2.73 (m, 2H), 2.64-2.53 (m, 1H). |
| 196 | 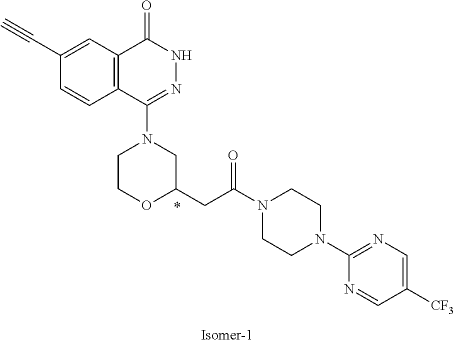 [SMILES: C#Cc1ccc2c(N3CCOC(CC(=O)N4CCN(c5ncc(C(F)(F)F)cn5)CC4)C3)n[nH]c(=O)c2c1] | 528.3 | ¹H NMR (400 MHz, methanol-d₄, ppm): δ 8.59 (s, 2H), 8.37 (d, J = 1.3 Hz, 1H), 8.05 (d, J = 8.4 Hz, 1H), 7.95 (dd, J = 8.4, 1.6 Hz, 1H), 4.31-4.20 (m, 1H), 4.08-3.83 (m, 7H), 3.81-3.59 (m, 4H), 3.57-3.51 (m, 1H), 3.36-3.30 (m, 1H), 3.06-2.94 (m, 1H), 2.87-2.72 (m, 2H), 2.67-2.55 (m, 1H). |
| 197 | 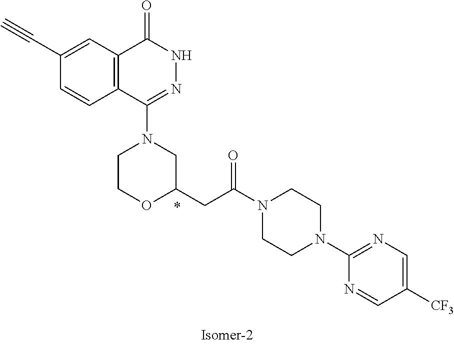 [SMILES: C#Cc1ccc2c(N3CCOC(CC(=O)N4CCN(c5ncc(C(F)(F)F)cn5)CC4)C3)n[nH]c(=O)c2c1] | 528.3 | ¹H NMR (400 MHz, methanol-d₄, ppm): δ 8.59 (s, 2H), 8.37 (d, J = 1.5 Hz, 1H), 8.05 (d, J = 8.4 Hz, 1H), 7.96 (dd, J = 8.4, 1.7 Hz, 1H), 4.30-4.20 (m, 1H), 4.09-3.81 (m, 7H), 3.80-3.60 (m, 4H), 3.57-3.51 (m, 1H), 3.36-3.30 (m, 1H), 3.05-2.96 (m, 1H), 2.87-2.73 (m, 2H), 2.66-2.55 (m, 1H). |
| 198 | 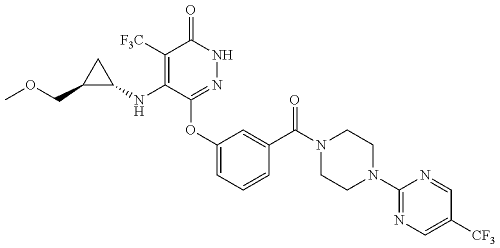 [SMILES: COC[C@H]1C[C@@H]1Nc1c(Oc2cccc(C(=O)N3CCN(c4ncc(C(F)(F)F)cn4)CC3)c2)n[nH]c(=O)c1C(F)(F)F] | 614.6 | 0.7TFA, ¹H NMR (400 MHz, methanol-d₄, ppm): δ 8.59 (s, 2H), 7.58-7.50 (m, 1H), 7.38- 7.29 (m, 3H), 4.10-3.74 (m, 6H), 3.62-3.50 (m, 2H), 3.46- 3.39 (m, 1H), 3.33 (s, 3H), 3.31-3.24 (m, 1H), 2.94-2.85 (m, 1H), 1.45-1.35 (m, 1H), 0.97-0.83 (m, 2H). ¹⁹F NMR (376 MHz, methanol-d₄, ppm) δ −56.12 (3F), −62.65 (3F). |
| 199 | 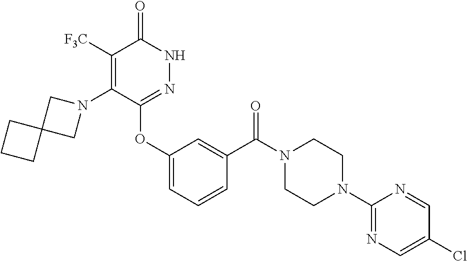 [SMILES: O=C(c1cccc(Oc2n[nH]c(=O)c(C(F)(F)F)c2N2CC3(CCC3)C2)c1)N1CCN(c2ncc(Cl)cn2)CC1] | 576.4 | ¹H NMR (400 MHz, DMSO-d₆, ppm): δ 11.56 (s, 1H), 8.47 (s, 2H), 7.54-7.50 (m, 1H), 7.37- 7.28 (m, 3H), 4.44 (s, 4H), 3.88-3.33 (m, 8H), 2.20-2.16 (m, 4H), 1.77-1.69 (m, 2H); ¹⁹F NMR (376 MHz, DMSO-d₆, ppm): δ −51.24 (3F). |
| 202 | 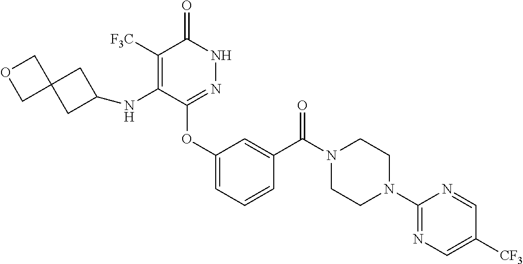 [SMILES: O=C(c1cccc(Oc2n[nH]c(=O)c(C(F)(F)F)c2NC2CC3(COC3)C2)c1)N1CCN(c2ncc(C(F)(F)F)cn2)CC1] | 626.6 | ¹H NMR (400 MHz, DMSO-d₆, ppm): δ 11.65 (s, 1H), 8.74 (s, 2H), 7.54-7.50 (m, 1H), 7.39-7.31 (m, 4H), 4.56-4.49 (m, 3H), 4.01-3.79 (m, 4H), 3.78-3.66 (m, 2H), 3.52-3.30 (m, 4H), 2.70-2.40 (m, 4H); ¹⁹F NMR (376 MHz, DMSO- d₆, ppm): δ −55.19 (3F), −59.29 (3F). |
| 203 | 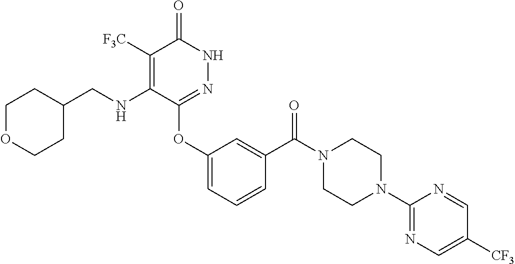 [SMILES: O=C(c1cccc(Oc2n[nH]c(=O)c(C(F)(F)F)c2NCC2CCOCC2)c1)N1CCN(c2ncc(C(F)(F)F)cn2)CC1] | 628.6 | ¹H NMR (400 MHz, DMSO-d₆, ppm): δ 11.72 (s, 1H), 8.74 (s, 2H), 7.55-7.51 (m, 1H), 7.36-7.30 (m, 3H), 7.09 (s, 3H), 4.02-3.65 (m, 8H), 3.45-3.20 (m, 6H), 1.98-1.91 (m, 1H), 1.60-1.50 (m, 2H) 1.20-1.08 (m, 2H). ¹⁹F NMR (376 MHz, DMSO-d₆, ppm): δ −54.74 (3F), −59.29 (3F). |
| 204 | 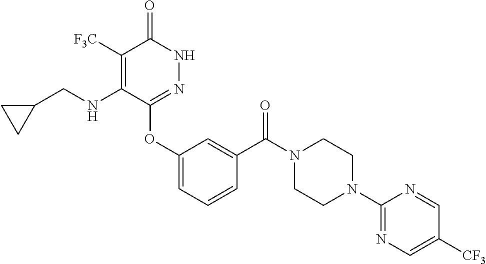 [SMILES: O=C(c1cccc(Oc2n[nH]c(=O)c(C(F)(F)F)c2NCC2CC2)c1)N1CCN(c2ncc(C(F)(F)F)cn2)CC1] | 584.4 | 0.2TFA salt, ¹H NMR (400 MHz, DMSO-d₆, ppm): δ 11.72 (s, 1H), 8.74 (s, 2H), 7.56-7.52 (m, 1H), 7.38-7.32 (m, 3H), 7.02-6.93 (m, 1H), 4.05-3.22 (m, 10H), 1.20-1.14 (m, 1H), 0.49-0.47 (m, 2H), 0.29-0.27 (m, 2H). ¹⁹F NMR (376 MHz, DMSO-d₆, ppm): δ −54.62 (3F), −59.30 (3F). |
| 205 | 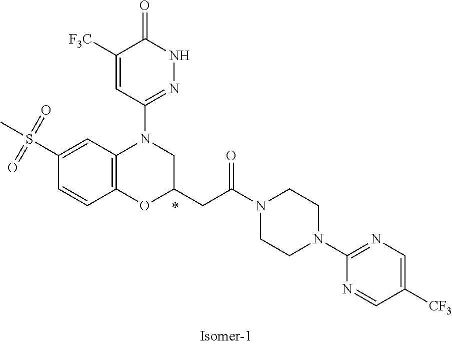 [SMILES: CS(=O)(=O)c1ccc2c(c1)N(c1cc(C(F)(F)F)c(=O)[nH]n1)CC(CC(=O)N1CCN(c3ncc(C(F)(F)F)cn3)CC1)O2] | 648.4 | SFC analysis: 99.06% ee; retention time: 3.935 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.60 (s, 2H), 8.06 (s, 1H), 7.66 (d, J = 2.0 Hz, 1H), 7.49 (dd, J = 8.6, 2.0 Hz, 1H), 7.11 (d, J = 8.6 Hz, 1H), 4.82-4.76 (m, 1H), 4.19 (dd, J = 13.3, 2.5 Hz, 1H), 4.06-3.89 (m, 4H), 3.75-3.64 (m, 4H), 3.62-3.52 (m, 1H), 3.10-3.00 (m, 4H), 2.89-2.79 (m, 1H). ¹⁹F NMR (376 MHz, methanol-d₄, ppm): δ −62.65 (3F), −68.68 (3F). |
| 206 | 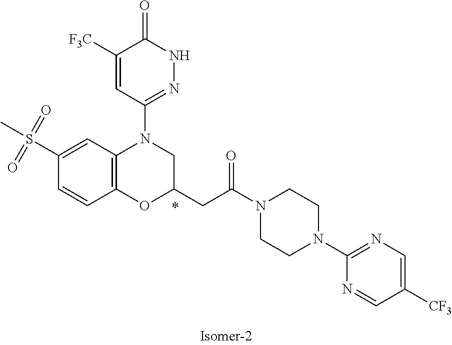 [SMILES: CS(=O)(=O)c1ccc2c(c1)N(c1cc(C(F)(F)F)c(=O)[nH]n1)CC(CC(=O)N1CCN(c3ncc(C(F)(F)F)cn3)CC1)O2] | 648.4 | SFC analysis: 90.74% ee; retention time: 4.353 min; column: DAICEL CHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.60 (s, 2H), 8.06 (s, 1H), 7.66 (d, J = 2.0 Hz, 1H), 7.49 (dd, J = 8.6, 2.0 Hz, 1H), 7.11 (d, J = 8.6 Hz, 1H), 4.82-4.76 (m, 1H), 4.19 (dd, J = 13.3, 2.5 Hz, 1H), 4.06-3.89 (m, 4H), 3.75-3.64 (m, 4H), 3.62-3.52 (m, 1H), 3.10-3.00 (m, 4H), 2.89-2.79 (m, 1H). ¹⁹F NMR (376 MHz, methanol-d₄, ppm): δ −62.64 (3F), −68.67 (3F). |
| 207 | 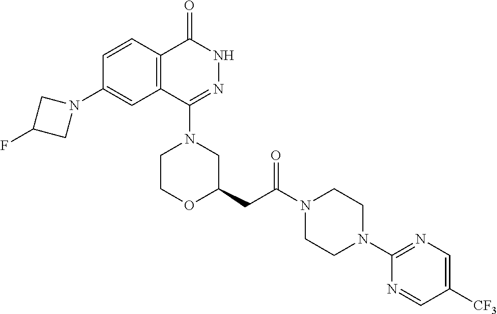 [SMILES: O=C(C[C@@H]1CN(c2n[nH]c(=O)c3ccc(N4CC(F)C4)cc23)CCO1)N1CCN(c2ncc(C(F)(F)F)cn2)CC1] | 577.2 | ¹H NMR (400 MHz, methanol-d₄, ppm): δ 8.58 (s, 2H), 8.23-8.13 (m, 1H), 7.02-6.92 (m, 1H), 6.94-6.84 (m, 1H), 5.75-5.40 (m, 1H), 4.55-4.35 (m, 2H), 4.30-4.10 (m, 3H), 4.08-3.82 (m, 6H), 3.80-3.58 (m, 5H), 3.33-3.25 (m, 2H), 3.09-2.94 (m, 1H), 2.90-2.80 (m, 1H), 2.70-2.53 (m, 2H). |
| 208 | 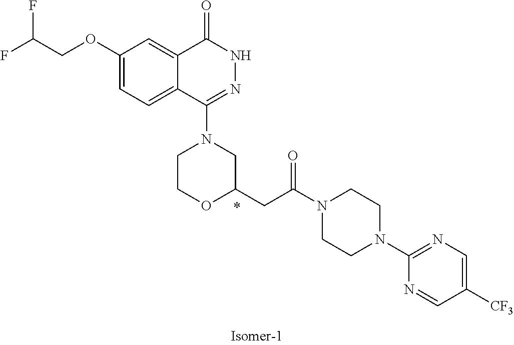 [SMILES: O=C(CC1CN(c2n[nH]c(=O)c3cc(OCC(F)F)ccc23)CCO1)N1CCN(c2ncc(C(F)(F)F)cn2)CC1] | 584.3 | SFC analysis: 100% ee; retention time: 7.440 min; column: DAICEL CHIRALPAK ® IA, 250 × 4.6 mm, 5 μm, A for n- Hexane and B for EtOH (0.2% DEA); flow rate: 1.0 mL/min. ¹H NMR (400 MHz, methanol-d₄, ppm): δ 8.59 (s, 2H), 8.03 (d, J = 8.9 Hz, 1H), 7.78 (d, J = 2.7 Hz, 1H), 7.55 (dd, J = 8.9, 2.7 Hz, 1H), 6.26 (tt, J = 54.8, 3.7 Hz, 1H), 4.43 (td, J = 13.7, 3.7 Hz, 2H), 4.33-4.17 (m, 1H), 4.10-3.82 (m, 6H), 3.82-3.58 (m, 4H), 3.52 (d, J = 12.3 Hz, 1H), 3.36- 3.31 (m, 1H), 3.04-2.91(m, 1H), 2.90-2.70 (m, 2H), 2.63- 2.52 (m, 1H). |
| 209 | 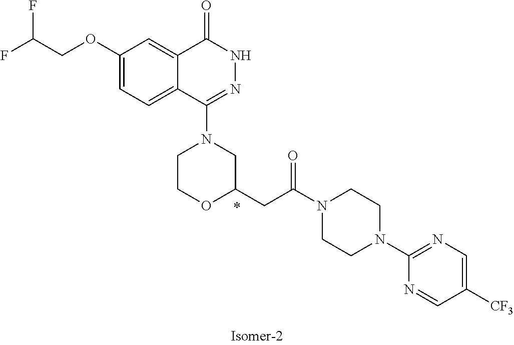 [SMILES: O=C(CC1CN(c2n[nH]c(=O)c3cc(OCC(F)F)ccc23)CCO1)N1CCN(c2ncc(C(F)(F)F)cn2)CC1] | 584.3 | SFC analysis: 99.59% ee; retention time: 11.456 min; column: DAICEL CHIRALPAK ® IA, 250 × 4.6 mm, 5 μm, A for n-Hexane and B for EtOH (0.2% DEA); flow rate: 1.0 mL/min. ¹H NMR (400 MHz, methanol-d₄, ppm): δ 8.59 (s, 2H), 8.03 (d, J = 8.9 Hz, 1H), 7.78 (d, J = 2.7 Hz, 1H), 7.55 (dd, J = 8.9, 2.7 Hz, 1H), 6.26 (tt, J = 54.8, 3.7 Hz, 1H), 4.43 (td, J = 13.7, 3.7 Hz, 2H), 4.30-4.18 (m, 1H), 4.10-3.83 (m, 6H), 3.82-3.57 (m, 4H), 3.52 (d, J = 12.3 Hz, 1H), 3.36-3.31 (m, 1H), 3.06-2.92 (m, 1H), 2.88-2.71 (m, 2H), 2.62-2.52 (m, 1H). |
| 210 | 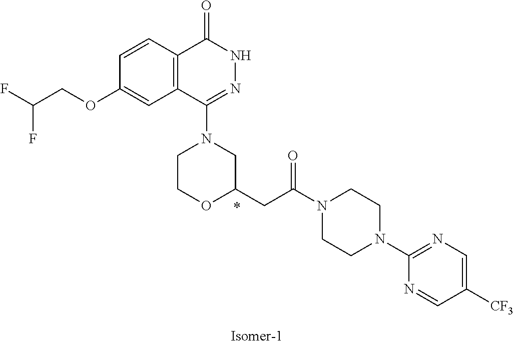 [SMILES: O=C(CC1CN(c2n[nH]c(=O)c3ccc(OCC(F)F)cc23)CCO1)N1CCN(c2ncc(C(F)(F)F)cn2)CC1] | 584.3 | SFC analysis: 99.25% ee; retention time: 12.993 min; column: DAICEL CHIRALPAK ® IA, 250 × 4.6 mm, 5 μm, A for n-Hexane and B for EtOH (0.2% DEA), 30%; flow rate: 1.0 mL/min. ¹H NMR (400 MHz, methanol-d₄, ppm): δ 8.59 (s, 2H), 8.32-8.26 (m, 1H), 7.60-7.30 (m, 2H), 6.28 (tt, J = 54.7, 3.6 Hz, 1H), 4.71-4.55 (m, 1H), 4.56-4.40 (m, 1H), 4.36-4.23 (m, 1H), 4.09-3.82 (m, 6H), 3.81-3.57 (m, 5H), 3.34-3.27 (m, 1H), 3.09-2.98 (m, 1H), 2.83 (dd, J = 15.5, 6.5 Hz, 1H), 2.74-2.65 (m, 1H), 2.64-2.54 (m, 1H). |
| 211 | 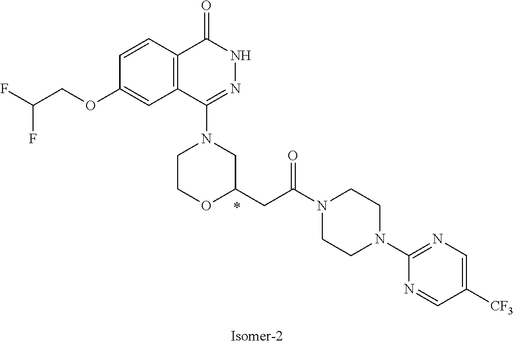 [SMILES: O=C(CC1CN(c2n[nH]c(=O)c3ccc(OCC(F)F)cc23)CCO1)N1CCN(c2ncc(C(F)(F)F)cn2)CC1] | 584.3 | SFC analysis: 93.04% ee; retention time: 16.088 min; column: DAICEL CHIRALPAK ® IA, 250 × 4.6 m, 5 μm, A for n-Hexane and B for EtOH (0.2% DEA), 30%; flow rate: 1.0 mL/min. ¹H NMR (400 MHz, methanol-d₄, ppm): δ 8.59 (s, 2H), 8.34-8.27 (m, 1H), 7.55-7.35 (m, 2H), 6.28 (tt, J = 54.7, 3.6 Hz, 1H), 4.70- 4.55 (m, 1H), 4.56-4.42 (m, 1H), 4.35-4.23 (m, 1H), 4.08-3.81 (m, 6H), 3.82-3.58 (m, 5H), 3.34-3.27 (m, 1H), 3.09-2.98 (m, 1H), 2.88-2.78 (m, 1H), 2.73-2.63 (m, 1H), 2.63-2.53 (m, 1H). |
| 212 | 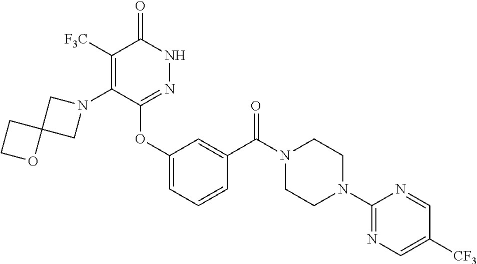 [SMILES: O=C(c1cccc(Oc2n[nH]c(=O)c(C(F)(F)F)c2N2CC3(CCO3)C2)c1)N1CCN(c2ncc(C(F)(F)F)cn2)CC1] | 612.4 | 0.3TFA salt, ¹H NMR (400 MHz, DMSO-d₆, ppm): δ 11.64 (s, 1H), 8.74 (s, 2H), 7.55-7.51 (m, 1H), 7.38-7.30 (m, 3H), 4.78-4.69 (m, 2H), 4.58-4.49 (m, 2H), 4.41-4.34 (m, 2H), 4.02-3.86 (m, 4H), 3.81-3.67 (m, 2H), 3.55-3.35 (m, 2H), 2.82 (t, 2H). ¹⁹F NMR (376 MHz, DMSO-dₕ₆, ppm): δ −51.41 (3F), −59.30 (3F). |
| 213 | 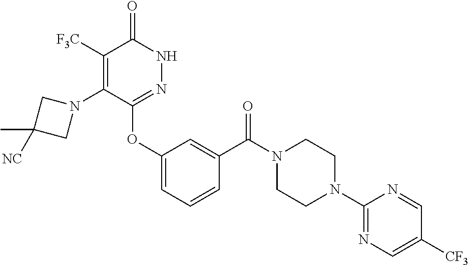 [SMILES: CC1(C#N)CN(c2c(Oc3cccc(C(=O)N4CCN(c5ncc(C(F)(F)F)cn5)CC4)c3)n[nH]c(=O)c2C(F)(F)F)C1] | 609.5 | ¹H NMR (400 MHz, DMSO-d₆, ppm): δ 11.77 (s, 1H), 8.74 (s, 2H), 7.55-7.51 (m, 1H), 7.39-7.30 (m, 3H), 4.80-4.70 (m, 2H), 4.45-4.35 (m, 2H), 4.02-3.85 (m, 4H), 3.82-3.69 (m, 2H), 3.55-3.39 (m, 2H), 1.65 (s, 3H). ¹⁹F NMR (376 MHz, DMSO-d₆, ppm): δ −51.95 (3F), −66.32 (3F). |
| 214 | 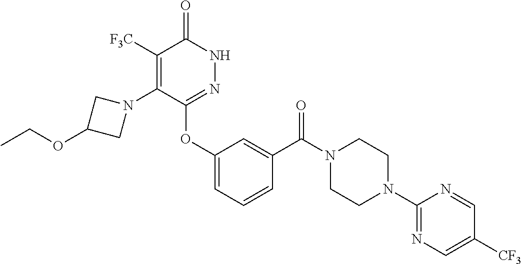 [SMILES: CCOC1CN(c2c(Oc3cccc(C(=O)N4CCN(c5ncc(C(F)(F)F)cn5)CC4)c3)n[nH]c(=O)c2C(F)(F)F)C1] | 614.6 | ¹H NMR (400 MHz, DMSO-d₆, ppm): δ 11.63 (s, 1H), 8.75 (s, 2H), 7.54-7.50 (m, 1H), 7.38-7.29 (m, 3H), 4.65-4.64 (m, 2H), 4.30-4.24 (m, 3H), 3.94- 3.85 (m, 4H), 3.76-3.70 (m, 2H), 3.50-3.41 (m, 4H), 1.14- 1.10 (m, 3H). ¹⁹F NMR (376 MHz, DMSO-d, ppm): δ −51.25 (3F), −59.30 (3F). |
| 215 | 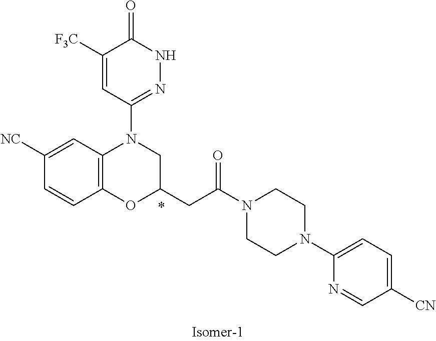 [SMILES: N#Cc1ccc(N2CCN(C(=O)CC3CN(c4cc(C(F)(F)F)c(=O)[nH]n4)c4cc(C#N)ccc4O3)CC2)nc1] | 551.4 | SFC analysis: 99.00% ee; retention time: 3.459 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 13.17-13.16 (m, 1H), 8.52 (s, 1H), 8.03 (s, 1H), 7.91 (d, J = 4.0 Hz, 1H), 7.67 (s, 1H), 7.34-7.30 (m, 1H), 7.04-7.02 (m, 1H), 6.97 (d, J = 12.0 Hz, 1H), 4.79-4.68 (m, 1H), 4.07-3.99 (m, 1H), 3.82-3.45 (m, 9H), 3.02-2.94 (m, 1H), 2.85-2.75 (m, 1H). ¹⁹F NMR (376 MHz, DMSO-d6, ppm): δ −65.97 (3F). |
| 216 | 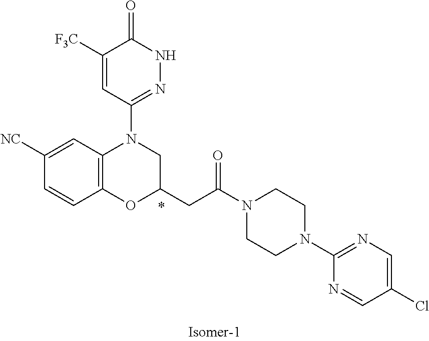 [SMILES: N#Cc1ccc2c(c1)N(c1cc(C(F)(F)F)c(=O)[nH]n1)CC(CC(=O)N1CCN(c3ncc(Cl)cn3)CC1)O2] | 561.4 | ¹H NMR (400 MHz, DMSO-d₆, ppm): δ 13.15 (s, 1H), 8.42 (s, 2H), 8.03 (s, 1H), 7.67 (s, 1H), 7.33 (d, J = 8.0 Hz, 1H), 7.04 (d, J = 8.0 Hz, 1H), 4.78-4.70 (m, 1H), 4.08-3.99 (m, 1H), 3.88-3.41 (m, 9H), 3.02-2.93 (m, 1H), 2.90-2.78 (m, 1H). ¹⁹F NMR (376 MHz, DMSO-d₆, ppm): δ −65.98 (3F). |
| 218 | 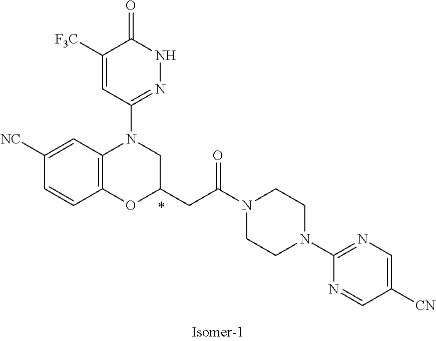 [SMILES: N#Cc1cnc(N2CCN(C(=O)CC3CN(c4cc(C(F)(F)F)c(=O)[nH]n4)c4cc(C#N)ccc4O3)CC2)nc1] | 552.4 | ¹H NMR (400 MHz, DMSO-d₆, ppm): δ 13.27-13.17 (m, 1H), 8.80 (s, 2H), 8.03 (s, 1H), 7.67-7.66 (m, 1H), 7.33-7.31 (m, 1H), 7.04-7.02 (m, 1H), 4.80-4.69 (m, 1H), 4.08-4.01 (m, 1H), 3.97-3.78 (m, 4H), 3.65-3.55 (m, 4H), 3.54-3.45 (m, 1H), 3.02-2.93 (m, 1H), 2.86-2.77 (m, 1H) ¹⁹F NMR (376 MHz, DMSO-d₆, ppm): δ −65.98 (3F). |
| 219 | 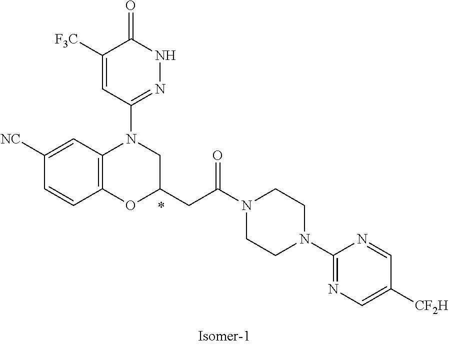 [SMILES: N#Cc1ccc2c(c1)N(c1cc(C(F)(F)F)c(=O)[nH]n1)CC(CC(=O)N1CCN(c3ncc(C(F)F)cn3)CC1)O2] | 577.4 | ¹H NMR (400 MHz, DMSO-d₆, ppm): δ 13.17 (s, 1H), 8.58 (s, 2H), 8.02 (s, 1H), 7.66 (d, J = 1.8 Hz, 1H), 7.32 (dd, J = 8.4, 2.0 Hz, 1H), 7.17-6.80 (m, 2H), 4.73 (t, J = 8.4 Hz, 1H), 4.09-4.01 (m, 1H), 3.90-3.72 (m, 4H), 3.64-3.46 (m, 5H), 3.02-2.92 (m, 1H), 2.90-2.78 (m, 1H). ¹⁹F NMR (376 MHz, DMSO-d₆, ppm): δ −65.97 (3F), −109.0 (2F). |
| 220 | 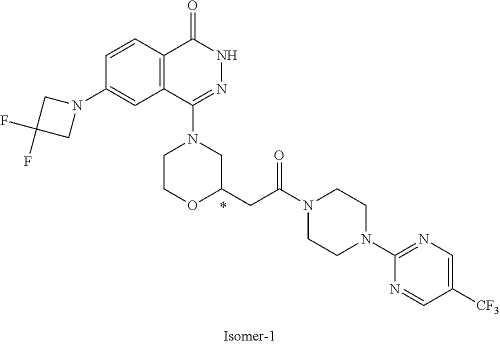 [SMILES: O=C(CC1CN(c2n[nH]c(=O)c3ccc(N4CC(F)(F)C4)cc23)CCO1)N1CCN(c2ncc(C(F)(F)F)cn2)CC1] | 595.3 | ¹H NMR (400 MHz, methanol-d₄, ppm): δ 8.58 (s, 2H), 8.23-8.13 (m, 1H), 7.02 (dd, J = 8.7, 2.3 Hz, 1H), 6.94-6.84 (m, 1H), 4.64-4.38 (m, 4H), 4.34-4.19 (m, 1H), 4.06-3.82 (m, 6H), 3.82-3.52 (m, 5H), 3.33-3.25 (m, 1H), 3.11-2.97 (m, 1H), 2.87-2.77 (m, 1H), 2.69-2.51 (m, 2H). |
| 221 | 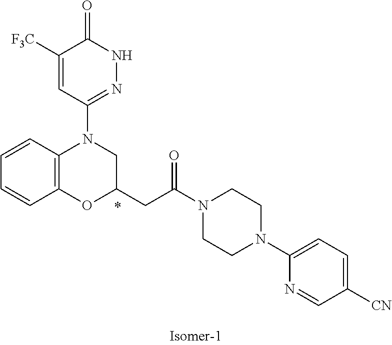 [SMILES: N#Cc1ccc(N2CCN(C(=O)CC3CN(c4cc(C(F)(F)F)c(=O)[nH]n4)c4ccccc4O3)CC2)nc1] | 526.2 | SFC analysis: 97.78% ee; retention time: 5.423 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.42 (d, J = 2.1 Hz, 1H), 7.98 (s, 1H), 7.76 (dd, J = 9.0, 2.3 Hz, 1H), 7.01-6.82 (m, 5H), 4.73-4.67 (m, 1H), 4.16 (dd, J = 13.2, 2.5 Hz, 1H), 3.83- 3.67 (m, 8H), 3.52 (dd, J = 13.2, 7.6 Hz, 1H), 3.05-2.95 (m, 1H), 2.83-2.73 (m, 1H). |
| 222 | 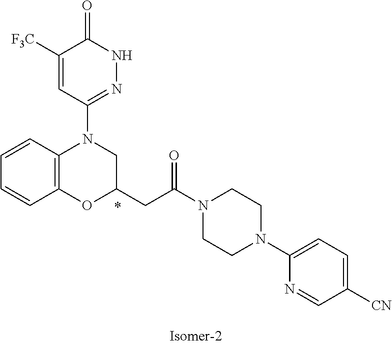 [SMILES: N#Cc1ccc(N2CCN(C(=O)CC3CN(c4cc(C(F)(F)F)c(=O)[nH]n4)c4ccccc4O3)CC2)nc1] | 526.2 | SFC analysis: 96.74% ee; retention time: 4.812 min; column: DAICEL CHIRALCEL ® AS, 100 × 3.0 mm, 3 μm, A for SO₂ and B for MeOH (0.1% DEA), 5% to 40%; pressure: 100 bar; flow rate: 1.5 mL/min. ¹H NMR (400 MHz, methanol-d₄, ppm): δ 8.42 (d, J = 2.2 Hz, 1H), 7.98 (s, 1H), 7.76 (dd, J = 9.0, 2.2 Hz, 1H), 7.02-6.82 (m, 5H), 4.72-4.66 (m, 1H, 4.16 (dd, J = 13.2, 2.6 Hz, 1H), 3.83- 3.67 (m, 8H), 3.52 (dd, J = 13.2, 7.6 Hz, 1H), 3.06-2.95 (m, 1H), 2.82-2.73 (m, 1H). |
| 223 | 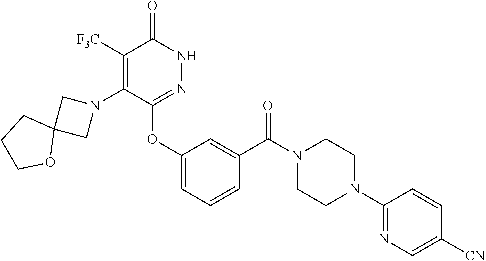 [SMILES: N#Cc1ccc(N2CCN(C(=O)c3cccc(Oc4n[nH]c(=O)c(C(F)(F)F)c4N4CC5(CCCO5)C4)c3)CC2)nc1] | 582.6 | ¹H NMR (400 MHz, methanol- d₄, ppm): δ 11.62 (s, 1H), 8.52 (s, 1H), 7.93-7.90 (m, 1H), 7.54-7.49 (m, 1H), 7.38- 7.29 (m, 3H), 6.96 (d, J = 12.0 Hz, 1H), 4.51-4.36 (m, 4H), 3.87-3.63 (m, 8H), 3.55-3.40 (m, 2H), 2.14-2.04 (m, 2H), 1.90-1.78 (m, 2H). |
| 224 | 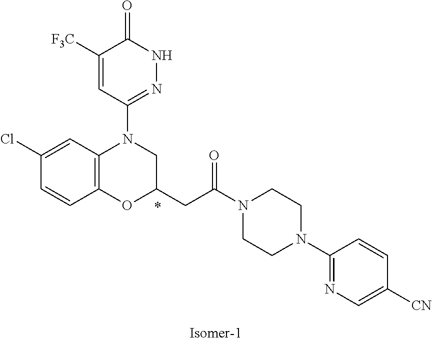 [SMILES: N#Cc1ccc(N2CCN(C(=O)CC3CN(c4cc(C(F)(F)F)c(=O)[nH]n4)c4cc(Cl)ccc4O3)CC2)nc1] | 560.4 | SFC analysis: 97.60% ee; retention time: 3.244 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. 0.6TFA salt, ¹H NMR (400 MHz, DMSO-d₆, ppm): δ 13.22 (s, 1H), 8.61-8.58 (m, 1H), 8.10 (s, 1H), 8.00-7.95 (m, 1H), 7.32-7.29 (m, 1H), 7.06-6.93 (m, 3H), 4.74-4.65 (m, 1H), 4.12-4.03 (m, 1H), 3.83-3.71 (m, 4H), 3.70-3.60 (m, 4H), 3.58-3.48 (m, 1H), 3.07-2.97 (m, 1H), 2.88-2.78 (m, 1H). ¹⁹F NMR (376 MHz, DMSO-d₆, ppm): δ −65.98 (3F). |
| 225 | 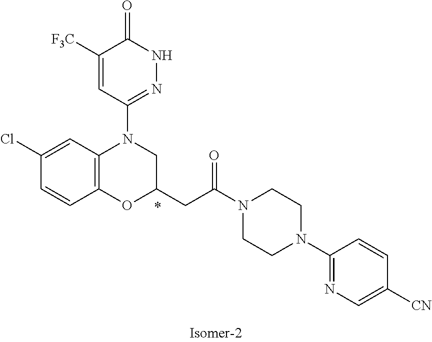 [SMILES: N#Cc1ccc(N2CCN(C(=O)CC3CN(c4cc(C(F)(F)F)c(=O)[nH]n4)c4cc(Cl)ccc4O3)CC2)nc1] | 560.4 | SFC analysis: 95.90% ee; retention time: 4.376 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. 1.0TFA salt, ¹H NMR (400 MHz, DMSO-d₆, ppm): δ 13.22 (s, 1H), 8.59 (s, 1H), 8.10 (s, 1H), 8.00-7.94 (m, 1H), 7.30 (s, 1H), 7.06-6.92 (m, 3H), 4.73- 4.64 (m, 1H), 4.11-4.03 (m, 1H), 3.85-3.71 (m, 4H), 3.70- 3.60 (m, 4H), 3.58-3.53 (m, 1H), 3.05-2.95 (m, 1H), 2.88- 2.77 (m, 1H); ¹⁹F NMR (376 MHz, DMSO-d₆, ppm): δ −65.98 (3F). |
| 226 | 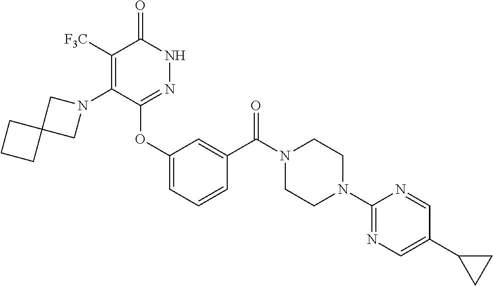 [SMILES: O=C(c1cccc(Oc2n[nH]c(=O)c(C(F)(F)F)c2N2CC3(CCC3)C2)c1)N1CCN(c2ncc(C3CC3)cn2)CC1] | 582.6 | ¹H NMR (400 MHz, DMSO-d₆, ppm): δ 11.56 (s, 1H), 8.20 (s, 2H), 7.53-7.49 (m, 1H), 7.36-7.28 (m, 3H), 4.44 (s, 4H), 3.76-3.69 (m, 6H), 3.40-3.25 (m, 2H), 2.20-2.16 (m, 4H), 2.01-1.98 (m, 1H), 1.80-1.78 (m, 2H), 0.88-0.84 (m, 2H), 0.66-0.62 (m, 2H). ¹⁹F NMR (376 MHz, DMSO-d₆, ppm): δ −51.25 (3F). |
| 229 | 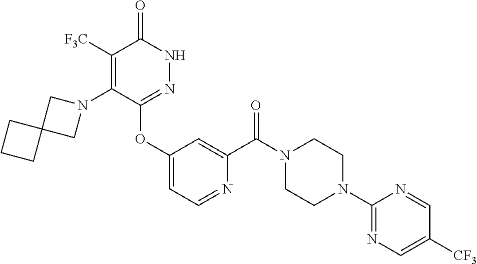 [SMILES: O=C(c1cc(Oc2n[nH]c(=O)c(C(F)(F)F)c2N2CC3(CCC3)C2)ccn1)N1CCN(c2ncc(C(F)(F)F)cn2)CC1] | 611.5 | 0.2TFA salt, ¹H NMR (400 MHz, DMSO-d₆, ppm): δ 11.83 (s, 1H), 8.75 (s, 2H), 8.62-8.61 (d, J = 5.6 Hz, 1H), 7.56- 7.55 (d, J = 1.9 Hz, 1H), 7.42-7.41 (m, 1H), 4.38 (s, 4H), 4.01-3.92 (m, 2H), 3.91-3.83 (m, 2H), 3.81-3.74 (m, 2H), 3.61-3.52 (m, 2H), 2.1-2.14 (t, J = 7.6 Hz, 4H), 1.74-1.70 (m, 2H). ¹⁹F NMR (376 MHz, DMSO-d₆, ppm): δ −51.37 (3F), −59.30 (3F). |
| 230 | 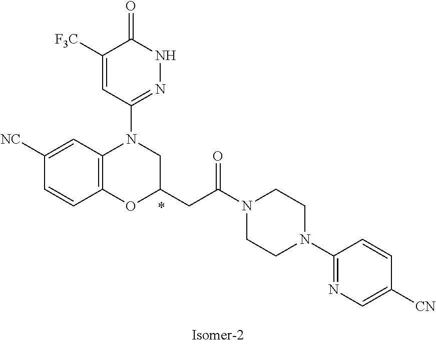 [SMILES: N#Cc1ccc(N2CCN(C(=O)CC3CN(c4cc(C(F)(F)F)c(=O)[nH]n4)c4cc(C#N)ccc4O3)CC2)nc1] | 551.5 | SFC analysis: 97.46% ee; retention time: 4.372 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 13.15 (s, 1H), 8.52 (d, J = 2.2 Hz, 1H), 8.01 (s, 1H), 7.89 (dd, J = 9.1, 2.3 Hz, 1H), 7.65 (s, 1H), 7.32 (dd, J = 8.4, 1.9 Hz, 1H), 7.02 (d, J = 8.4 Hz, 1H), 6.99-6.92 (m, 1H), 4.79-4.68 (m, 1H), 4.05 (dd, J = 13.2, 2.4 Hz, 1H), 3.81-3.47 (m, 9H), 3.02-2.93 (m, 1H), 2.85-2.77 (m, 1H). ¹⁹F NMR (376 MHz, DMSO-d₆): δ −65.96 (s, 3H). |
| 231 | 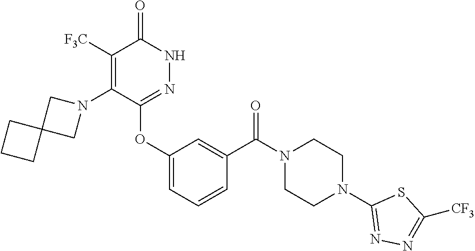 [SMILES: O=C(c1cccc(Oc2n[nH]c(=O)c(C(F)(F)F)c2N2CC3(CCC3)C2)c1)N1CCN(c2nnc(C(F)(F)F)s2)CC1] | 616.4 | 0.7TFA salt, ¹H NMR (400 MHz, DMSO-d₆, ppm): δ 11.56 (s, 1H), 7.59-7.49 (m, 1H), 7.41-7.26 (m, 3H), 4.44 (s, 4H), 3.82-3.39 (m, 8H), 2.20-2.10 (m, 4H), 1.77-1.65 (m, 2H). ¹⁹F NMR (376 MHz, DMSO-d₆, ppm): δ −51.24 (3F), −58.42 (3F). |
| 232 | 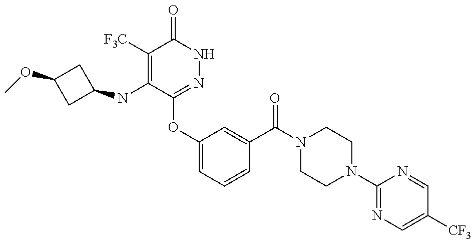 [SMILES: CO[C@H]1C[C@@H](Nc2c(Oc3cccc(C(=O)N4CCN(c5ncc(C(F)(F)F)cn5)CC4)c3)n[nH]c(=O)c2C(F)(F)F)C1] | 615.4 | 0.4TFA salt, ¹H NMR (400 MHz, DMSO-d₆, ppm): δ 12.65 (s, 1H), 8.74 (s, 2H), 7.54 (t, J = 7.8 Hz, 1H), 7.44-7.35 (m, 2H), 7.34-7.31 (m, 1H), 4.92-4.78 (m, 1H), 3.95-3.85 (m, 4H), 3.80-3.61 (m, 2H), 3.53-3.40 (m, 3H), 3.13 (s, 3H), 2.85-2.75 (m, 2H), 2.15-2.01 (m, 2H). ¹⁹F NMR (376 MHz, DMSO-d₆, ppm): δ −58.39 (3F), −59.30 (3F). |
| 233 | 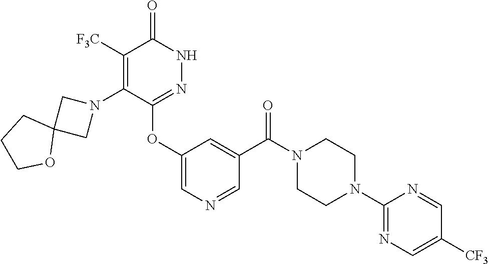 [SMILES: O=C(c1cncc(Oc2n[nH]c(=O)c(C(F)(F)F)c2N2CC3(CCCO3)C2)c1)N1CCN(c2ncc(C(F)(F)F)cn2)CC1] | 627.5 | 1.7TFA salt, ¹H NMR (400 MHz, DMSO-d₆, ppm): δ 11.69 (s, 1H), 8.77 (s, 2H), 8.67 (s, 1H), 8.56 (s, 1H), 7.95 (s, 1H), 4.60-4.40 (m, 4H), 4.05-3.87 (m, 4H), 3.83-3.60 (m, 6H), 2.15-2.07 (m, 2H), 1.90-1.80 (m, 2H). ¹⁹F NMR (376 MHz, DMSO-d₆, ppm): δ −51.25 (3F), −59.29 (3F). |
| 234 | 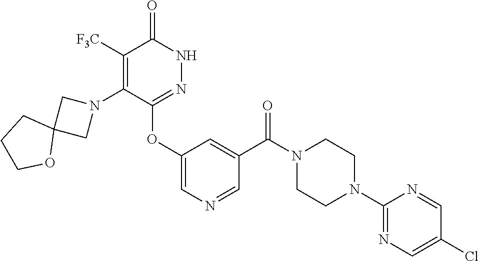 [SMILES: O=C(c1cncc(Oc2n[nH]c(=O)c(C(F)(F)F)c2N2CC3(CCCO3)C2)c1)N1CCN(c2ncc(Cl)cn2)CC1] | 593.5 | 1.2TFA salt, ¹H NMR (400 MHz, DMSO-d₆, ppm): δ 11.67 (s, 1H), 8.66 (s, 1H), 8.53 (s, 1H), 8.47 s, 2H), 7.93 (s, 1H), 4.54-4.42 (m, 4H), 3.90-3.65 (m, 10H), 2.15-2.04 (m, 2H), 1.90-1.80 (m, 2H). ¹⁹F NMR (376 MHz, DMSO-d₆, ppm): δ −51.25 (3F). |
| 235 | 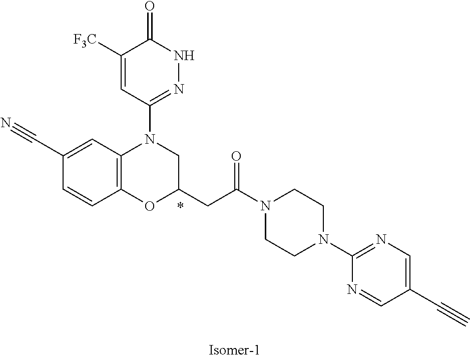 [SMILES: C#Cc1cnc(N2CCN(C(=O)CC3CN(c4cc(C(F)(F)F)c(=O)[nH]n4)c4cc(C#N)ccc4O3)CC2)nc1] | 551.4 | ¹H NMR (400 MHz, DMOS-d₆, ppm): δ 13.17 (s, 1H), 8.51 (s, 2H), 8.02 (s, 1H), 7.65 (d, J = 1.6 Hz, 1H), 7.32 (dd, J = 8.4, 2.0 Hz, 1H), 7.02 (d, J = 8.4 Hz, 1H), 4.72-4.68 (m, 1H), 4.31 (s, 1H), 4.02 (dd, J = 13.2, 2.4 Hz, 1H), 3.83-3.70 (m, 4H), 3.58-3.47 (m, 5H), 3.01-2.93 (m, 1H), 2.85-2.75 (m, 1H). ¹⁹F NMR (376 MHz, DMSO-d₆, ppm): δ −65.97 (3F). |
| 237 | 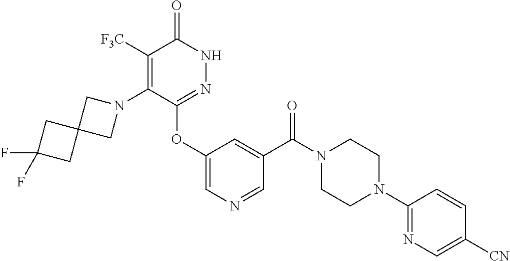 [SMILES: N#Cc1ccc(N2CCN(C(=O)c3cncc(Oc4n[nH]c(=O)c(C(F)(F)F)c4N4CC5(C4)CC(F)(F)C5)c3)CC2)nc1] | 603.4 | 1.5TFA salt, ¹H NMR (400 MHz, DMSO-d₆, ppm): δ 11.65 (s, 1H), 8.65 (d, J = 2.6 Hz, 1H), 8.55-8.51 (m, 2H), 7.93- 7.90 (m, 2H), 6.95 (d, J = 9.0 Hz, 1H), 4.60 (s, 4H), 3.85- 3.55 (m, 6H), 3.53-3.41 (m, 2H), 2.89 (t, J = 12.5 Hz, 4H). ¹⁹F NMR (376 MHz, DMSO-d₆, ppm): δ −51.40 (3F), −91.00 (2F). |
| 238 | 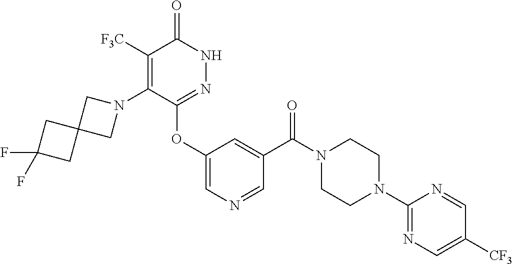 [SMILES: O=C(c1cncc(Oc2n[nH]c(=O)c(C(F)(F)F)c2N2CC3(C2)CC(F)(F)C3)c1)N1CCN(c2ncc(C(F)(F)F)cn2)CC1] | 647.2 | 1.8TFA salt, ¹H NMR (400 MHz, DMSO-d₆, ppm): δ 11.67 (s, 1H), 8.75 (s, 2H), 8.65 (d, J = 2.6 Hz, 1H), 8.55 (d, J = 1.7 Hz, 1H), 7.92-7.91 (m, 1H), 4.60 (s, 4H), 3.96-3.91 (m, 4H), 3.75-3.62 (m, 2H), 3.55-3.45 (m, 2H), 2.89 (t, J = 12.5 Hz, 4H). ¹⁹F NMR (376 MHz, DMSO-d₆, ppm): δ −51.40 (3F), −59.30 (3F), −91.00 (2F). |
| 239 | 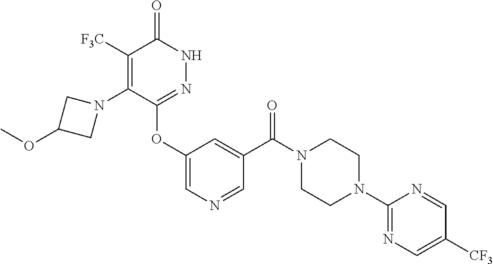 [SMILES: COC1CN(c2c(Oc3cncc(C(=O)N4CCN(c5ncc(C(F)(F)F)cn5)CC4)c3)n[nH]c(=O)c2C(F)(F)F)C1] | 601.4 | ¹H NMR (400 MHz, DMSO-d₆, ppm): δ 11.67 (s, 1H), 8.75 (s, 2H), 8.66 (d, J = 2.6 Hz, 1H), 8.54 (d, J = 1.7 Hz, 1H), 7.94-7.92 (m, 1H), 4.69-4.64 (m, 2H), 4.31-4.24 (m, 2H), 4.23-4.19 (m, 1H), 4.01-3.86 (m, 4H), 3.80-3.70 (m, 2H), 3.50-3.40 (m, 2H), 3.24 (s, 3H). ¹⁹F NMR (376 MHz, DMSO-d₆, ppm): δ −51.28 (3F), −59.29 (3F). |
| 240 | 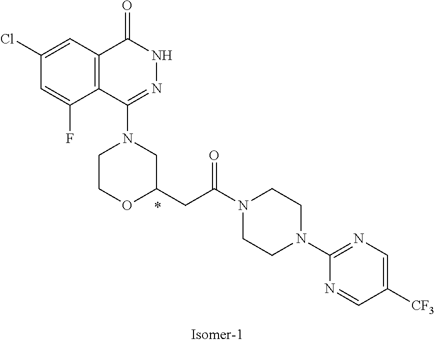 [SMILES: O=C(CC1CN(c2n[nH]c(=O)c3cc(Cl)cc(F)c23)CCO1)N1CCN(c2ncc(C(F)(F)F)cn2)CC1] | 556.2 | SFC analysis: 100% ee; retention time: 11.280 min; column: DAICELCHIRALPAK ® IA, 250 × 4.6 mm, 5 μm, A for n-Hexane and B for EtOH/ACN = 6.4; flow rate: 1.0 mL/min. ¹H NMR (400 MHz, DMSO-d₆, ppm): δ 12.40 (s, 1H), 8.73 (s, 2H), 8.16-7.87 (m, 2H), 4.05 (d, J = 7.2 Hz, 1H), 3.98-3.75 (m, 5H), 3.69 (t, J = 10.2 Hz, 1H), 3.65-3.53 (m, 4H), 3.40-3.34 (m, 1H), 3.21 (d, J = 12.1 Hz, 1H), 2.85- 2.66 (m, 2H), 2.65-2.50 (m, 2H). |
| 241 | 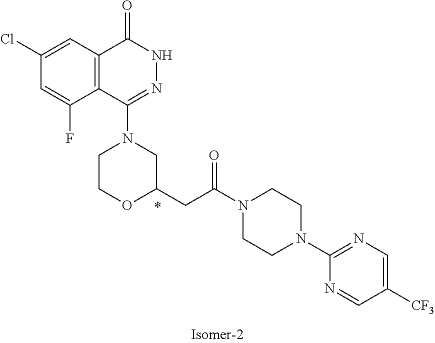 [SMILES: O=C(CC1CN(c2n[nH]c(=O)c3cc(Cl)cc(F)c23)CCO1)N1CCN(c2ncc(C(F)(F)F)cn2)CC1] | 556.2 | SFC analysis: 100% ee; retention time: 25.486 min; column: DAICELCHIRALPAK ® IA, 250 × 4.6 mm, 5 μm, A for n-Hexane and B for EtOH/ACN = 6/4; flow rate: 1.0 mL/min. ¹H NMR (400 MHz, DMSO-d₆, ppm): δ 12.40 (s, 1H), 8.73 (s, 2H), 8.18- 7.88 (m, 2H), 4.05 (d, J = 7.3 Hz, 1H), 3.96-3.78 (m, 5H), 3.69 (t, J = 10.4 Hz, 1H), 3.64-3.54 (m, 4H), 3.38 (d, J = 12.3 Hz, 1H), 3.21 (d, J = 12.5 Hz, 1H), 2.86-2.67 (m, 2H), 2.65-2.52 (m, 2H). |
| 242 | 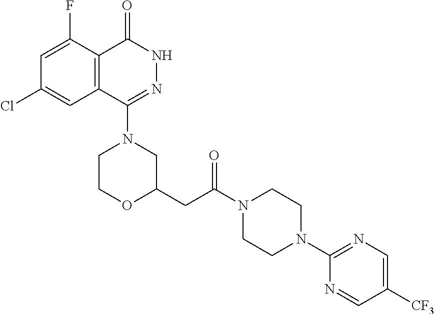 [SMILES: O=C(CC1CN(c2n[nH]c(=O)c3c(F)cc(Cl)cc23)CCO1)N1CCN(c2ncc(C(F)(F)F)cn2)CC1] | 556.2 | ¹H NMR (400 MHz, methanol-d₄, ppm): δ 8.59 (s, 2H), 7.84 (s, 1H), 7.64 (dd, J = 10.7, 1.7 Hz, 1H), 4.31-4.17 (m, 1H), 4.10-3.82 (m, 6H), 3.83-3.59 (m, 4H), 3.43 (d, J 32 12.3 Hz, 1H, 3.25 (d, J = 12.6 Hz, 1H), 3.05-2.93 (m, 1H), 2.89-2.73 (m, 2H), 2.63-2.53 (m, 1H). |
| 243 | 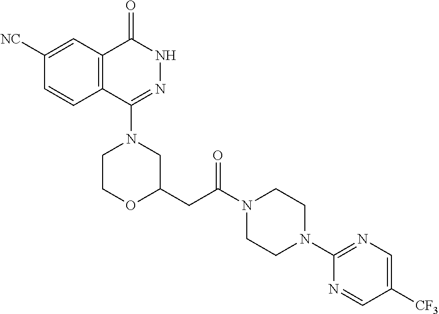 [SMILES: N#Cc1ccc2c(N3CCOC(CC(=O)N4CCN(c5ncc(C(F)(F)F)cn5)CC4)C3)n[nH]c(=O)c2c1] | 529.2 | ¹H NMR (400 MHz, methanol-d₄, ppm): δ 8.65 (s, 1H), 8.59 (s, 2H), 8.24-8.14 (m, 2H), 4.34-4.17 (m, 1H), 4.09-3.83 (m, 6H), 3.81-3.60 (m, 4H), 3.56-3.45 (m, 1H), 3.36-3.33 (m, 1H), 3.08-2.97 (m, 1H), 2.89-2.75 (m, 2H), 2.64-2.53 (m, 1H). |
| 244 | 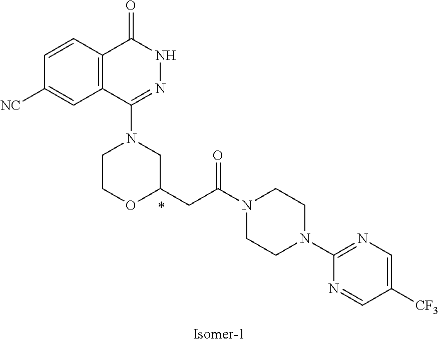 [SMILES: N#Cc1ccc2c(=O)[nH]nc(N3CCOC(CC(=O)N4CCN(c5ncc(C(F)(F)F)cn5)CC4)C3)c2c1] | 529.3 | SFC analysis: 94.45% ee; retention time: 23.454 min; column: DAICELCHIRALPAK ® IC, 250 × 4.6 mm, 5 μm, A for n-Hexane and B for EtOH (0.2% DEA), 60%; flow rate: 1.0 mL/min. ¹H NMR (400 MHz, methanol-d₄, ppm): δ 8.59 (s, 2H), 8.47 (d, J = 8.3 Hz, 1H), 8.40 (s, 1H), 8.12 (dd, J = 8.3, 1.5 Hz, 1H), 4.34-4.23 (m, 1H), 4.05-3.86 (m, 6H), 3.81-3.62 (m, 4H), 3.55-3.46 (m, 1H), 3.36-3.31 (m, 1H), 3.10-2.98 (m, 1H), 2.87-2.78 (m, 2H), 2.65-2.55 (m, 1H). |
| 245 | 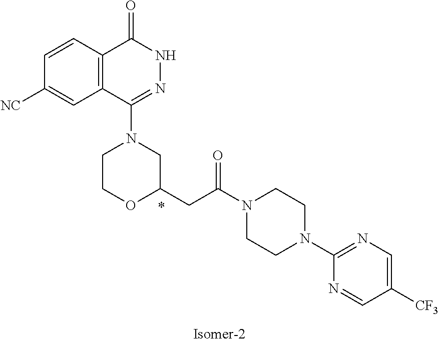 [SMILES: N#Cc1ccc2c(=O)[nH]nc(N3CCOC(CC(=O)N4CCN(c5ncc(C(F)(F)F)cn5)CC4)C3)c2c1] | 529.2 | SFC analysis: 100% ee; retention time: 35.932 min; column: DAICELCHIRALPAK ® IC, 250 × 4.6 mm, 5 μm, A for n-Hexane and B for EtOH (0.2% DEA), 60%; flow rate: 1.0 mL/min. ¹H NMR (400 MHz, methanol-d₄, ppm): δ 8.59 (s, 2H), 8.47 (d, J = 8.2 Hz, 1H), 8.40 (s, 1H), 8.12 (dd, J = 8.3, 1.5 Hz, 1H), 4.35-4.23 (m, 1H), 4.07-3.85 (m, 6H), 3.80-3.60 (m, 4H), 3.54-3.46 (m, 1H), 3.35-3.31 (m, 1H), 3.08-2.99 (m, 1H), 2.87-2.77 (m, 2H), 2.65-2.56 (m, 1H). |
| 246 | 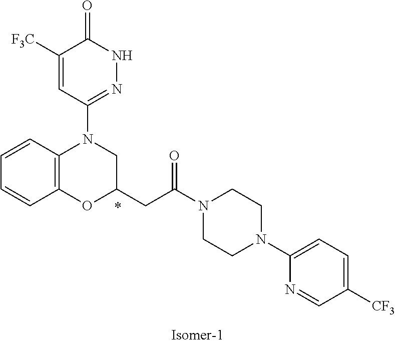 [SMILES: O=C(CC1CN(c2cc(C(F)(F)F)c(=O)[nH]n2)c2ccccc2O1)N1CCN(c2ccc(C(F)(F)F)cn2)CC1] | 569.2 | SFC analysis: 99.46% ee; retention time: 3.950 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.37 (s, 1H), 7.99 (s, 1H), 7.75 (dd, J = 9.1, 2.4 Hz, 1H), 7.09-6.81 (m, 5H), 4.77-4.65 (m, 1H), 4.17 (d, J = 13.2 Hz, 1H), 3.87-3.63 (m, 8H), 3.53 (dd, J = 12.7, 7.6 Hz, 1H), 3.07-2.95 (m, 1H), 2.86-2.76 (m, 1H). |
| 247 | 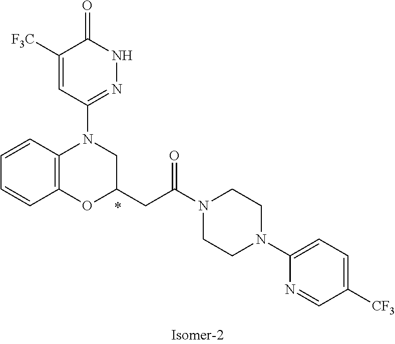 [SMILES: O=C(CC1CN(c2cc(C(F)(F)F)c(=O)[nH]n2)c2ccccc2O1)N1CCN(c2ccc(C(F)(F)F)cn2)CC1] | 569.2 | SFC analysis: 95.92% ee; retention time: 3.512 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.37 (s, 1H), 7.98 (s, 1H), 7.81- 7.68 (m, 1H), 7.08-6.83 (m, 5H), 4.76-4.63 (m, 1H), 4.17 (dd, J = 13.2, 2.4 Hz, 1H), 3.85-3.63 (m, 8H), 3.52 (dd, J = 13.2, 7.6 Hz, 1H), 3.09-2.98 (m, 1H), 2.86-2.73 (m, 1H) |
| 248 | 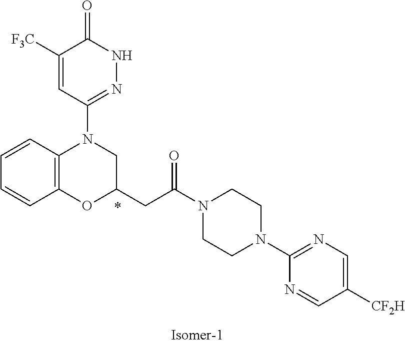 [SMILES: O=C(CC1CN(c2cc(C(F)(F)F)c(=O)[nH]n2)c2ccccc2O1)N1CCN(c2ncc(C(F)F)cn2)CC1] | 552.3 | SFC analysis: 100% ee; retention time: 4.212 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.49 (s, 2H), 7.98 (s, 1H), 7.02-6.85 (m, 4H), 6.73 (t, J = 49.0 Hz, 1H), 4.73-4.65 (m, 1H), 4.21- 4.11 (m, 1H), 4.03-3.85 (m, 4H), 3.78-3.62 (m, 4H), 3.53 (dd, J = 13.3, 7.6 Hz, 1H), 3.05-2.98 (m, 1H), 2.87-2.77 (m, 1H). |
| 249 | 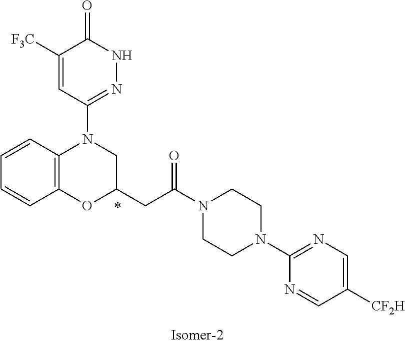 [SMILES: O=C(CC1CN(c2cc(C(F)(F)F)c(=O)[nH]n2)c2ccccc2O1)N1CCN(c2ncc(C(F)F)cn2)CC1] | 552.3 | SFC analysis: 100% ee; retention time: 3.741 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.49 (s, 2H), 7.98 (s, 1H), 7.05-6.85 (m, 4H), 6.73 (t, J = 49.0 Hz, 1H), 4.73-4.65 (m, 1H), 4.21- 4.11 (m, 1H), 4.04-3.85 (m, 4H), 3.78-3.62 (m, 4H), 3.53 (dd, J = 13.3, 7.6 Hz, 1H), 3.07-2.97 (m, 1H), 2.86-2.77 (m, 1H). |
| 250 | 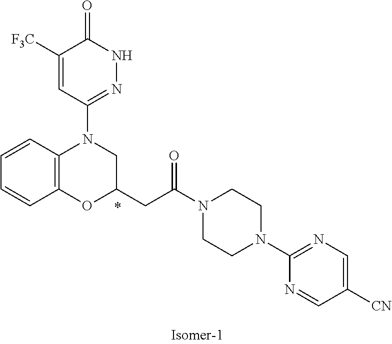 [SMILES: N#Cc1cnc(N2CCN(C(=O)CC3CN(c4cc(C(F)(F)F)c(=O)[nH]n4)c4ccccc4O3)CC2)nc1] | 527.2 | SFC analysis: 100% ee; retention time: 1.020 min; column: DAICEL CHIRALPAK ® AD, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.98 (s, 1H), 7.05-6.83 (m, 4H), 4.74- 4.66 (m, 1H), 4.16 (dd, J = 13.2, 2.6 Hz, 1H), 4.07-3.90 (m, 4H), 3.81-3.61 (m, 4H), 3.52 (dd, J = 13.2, 7.6 Hz, 1H), 3.05-2.97 (m, 1H), 2.86-2.76 (m, 1H). |
| 251 | 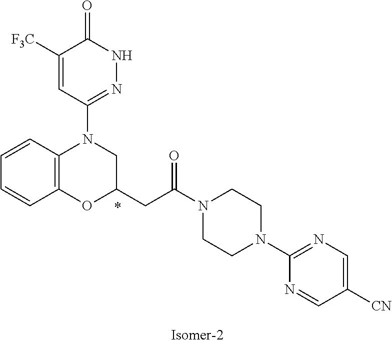 [SMILES: N#Cc1cnc(N2CCN(C(=O)CC3CN(c4cc(C(F)(F)F)c(=O)[nH]n4)c4ccccc4O3)CC2)nc1] | 527.2 | SFC analysis: 93.32% ee; retention time: 2.197 min; column: DAICEL CHIRALPAK ® AD, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.98 (s, 1H), 7.04- 6.83 (m, 4H), 4.78-4.67 (m, 1H), 4.16 (dd, J = 13.2, 2.6 Hz, 1H), 4.04-3.89 (m, 4H), 3.78-3.63 (m, 4H), 3.52 (dd, J = 13.2, 7.6 Hz, 1H), 3.07-2.99 (m, 1H), 2.87-2.76 (m, 1H). |
| 252 | 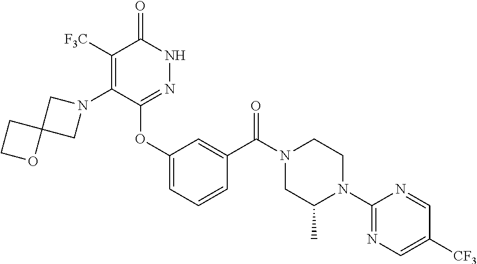 [SMILES: C[C@@H]1CN(C(=O)c2cccc(Oc3n[nH]c(=O)c(C(F)(F)F)c3N3CC4(CCO4)C3)c2)CCN1c1ncc(C(F)(F)F)cn1] | 626.5 | 0.4TFA salt, ¹H NMR (400 MHz, DMSO-d₆, ppm): δ 11.64 (s, 1H), 8.73 (s, 2H), 7.59-7.52 (m, 1H), 7.49-7.25 (m, 3H), 5.12-4.78 (m, 1H), 4.77-4.68 (m, 2H), 4.65-4.34 (m, 6H), 3.80-2.98 (m, 4H), 2.89-2.81 (m, 2H), 1.25-1.05 (m, 3H). ¹⁹F NMR (376 MHz, DMSO-d₆): δ −51.40 (3F), −59.30 (3F). |
| 253 | 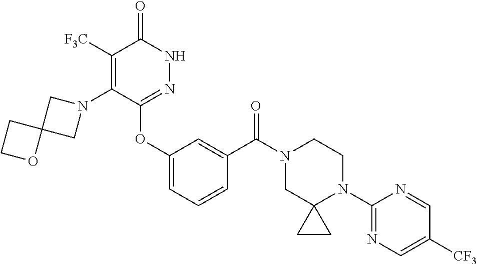 [SMILES: O=C(c1cccc(Oc2n[nH]c(=O)c(C(F)(F)F)c2N2CC3(CCO3)C2)c1)N1CCN(c2ncc(C(F)(F)F)cn2)C2(CC2)C1] | 638.4 | 0.8TFA salt, ¹H NMR (400 MHz, DMSO-d₆, ppm): δ 11.63 (s, 1H), 8.82 (s, 2H), 7.59-7.48 (m, 1H), 7.38-7.18 (m, 3H), 4.79-4.68 (m, 2H), 4.59-4.47 (m, 2H), 4.39-4.31 (m, 2H), 4.13-3.98 (m, 2H), 3.82-3.59 (m, 2H), 3.52-3.35 (m, 2H), 2.84 (t, 2H), 1.15-0.80 (m, 4H). ¹⁹F NMR (376 MHz, DMSO-d₆): δ −51.41 (3F), −59.54 (3F). |
| 254 | 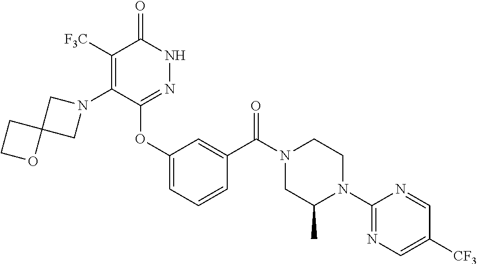 [SMILES: C[C@H]1CN(C(=O)c2cccc(Oc3n[nH]c(=O)c(C(F)(F)F)c3N3CC4(CCO4)C3)c2)CCN1c1ncc(C(F)(F)F)cn1] | 626.4 | ¹H NMR (400 MHz, DMSO-d₆, ppm): δ 11.64 (s, 1H), 8.75 (s, 2H), 7.58-7.51 (m, 1H), 7.48-7.22 (m, 3H), 5.12-4.80 (m, 1H), 4.77-4.67 (m, 2H), 4.65-4.32 (m, 6H), 3.81-3.01 (m, 4H), 2.88-2.77 (m, 2H), 1.25-1.10 (m, 3H). |
| 255 | 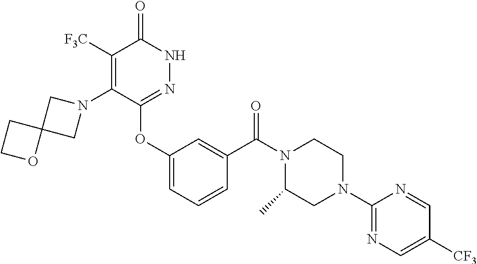 [SMILES: C[C@H]1CN(c2ncc(C(F)(F)F)cn2)CCN1C(=O)c1cccc(Oc2n[nH]c(=O)c(C(F)(F)F)c2N2CC3(CCO3)C2)c1] | 626.5 | ¹H NMR (400 MHz, DMSO-d₆, ppm): δ 11.64 (s, 1H), 8.73 (s, 2H), 7.59-7.51 (m, 1H), 7.38-7.26 (m, 3H), 4.75-4.68 (m, 2H), 4.65-4.51 (m, 3H), 4.38- 4.34 (m, 2H), 4.05-4.00 (m, 1H), 3.41-3.10 (m, 5H), 2.86- 2.82 (m, 2H), 1.19-1.12 (m, 3H). ¹⁹F NMR (376 MHz, DMSO-d₆): δ −51.41 (3F), −59.28 (3F). |
| 256 | 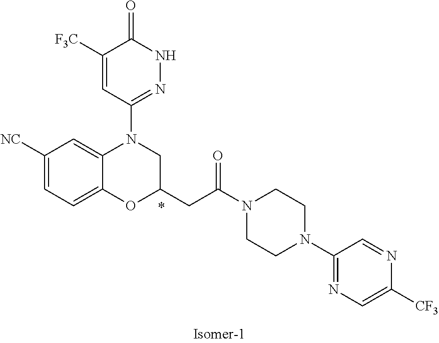 [SMILES: N#Cc1ccc2c(c1)N(c1cc(C(F)(F)F)c(=O)[nH]n1)CC(CC(=O)N1CCN(c3cnc(C(F)(F)F)cn3)CC1)O2] | 595.4 | ¹H NMR (400 MHz, DMSO-d₆, ppm): δ 13.15 (s, 1H), 8.52 (s, 1H), 8.45 (s, 1H), 8.03 (s, 1H), 7.67 (d, J = 1.6 Hz, 1H), 7.32 (dd, J = 8.4, 1.6 Hz, 1H), 7.03 (d, J = 8.4 Hz, 1H), 4.73-4.69 (m, 1H), 4.10-4.00 (m, 1H), 3.80-3.68 (m, 4H), 3.62-3.55 (m, 4H), 3.55- 3.45 (m, 1H), 3.01- 2.92 (dd, J = 16.0, 6.4 Hz, 1H), 2.190-2.79 (m, 1H). ¹⁹F NMR (376 MHz, DMSO-d₆, ppm): δ −64.66 (3F), −65.98 (3F). |
| 257 | 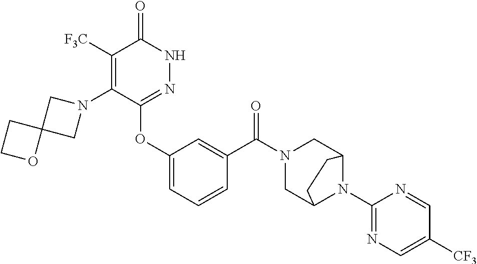 [SMILES: O=C(c1cccc(Oc2n[nH]c(=O)c(C(F)(F)F)c2N2CC3(CCO3)C2)c1)N1CC2CCC(C1)N2c1ncc(C(F)(F)F)cn1] | 638.5 | ¹H NMR (400 MHz, DMSO-d₆, ppm): δ 11.62 (s, 1H), 8.75 (s, 2H), 7.57-7.43 (m, 1H), 7.35-7.20 (m, 3H), 4.94-4.69 (m, 4H), 4.58-4.4.36 (m, 5H), 3.55-3.30 (m, 2H), 3.10-2.98 (m, 1H), 2.84 (t, J = 7.6 Hz, 2H), 2.05-1.89 (m, 2H), 1.80-1.68 (m, 2H). ¹⁹F NMR (376 MHz, DMSO-d₆, ppm): δ −51.44 (3F), −59.34 (3F). |
| 258 | 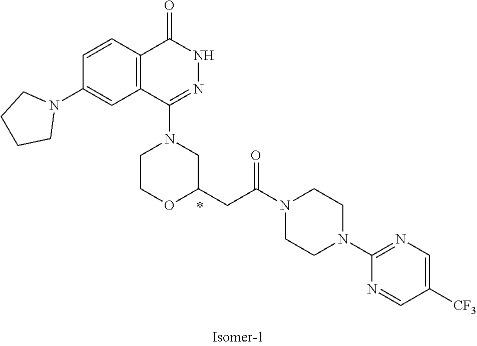 [SMILES: O=C(CC1CN(c2n[nH]c(=O)c3ccc(N4CCCC4)cc23)CCO1)N1CCN(c2ncc(C(F)(F)F)cn2)CC1] | 573.3 | ¹H NMR (400 MHz, methanol- d₄, ppm): δ 8.59 (s, 2H), 8.10 (d, J = 8.9 Hz, 1H), 7.07 (dd, J = 9.0, 2.3 Hz, 1H), 6.87 (d, J = 2.3 Hz, 1H), 4.31-4.18 (m, 1H), 4.07-3.82 (m, 6H), 3.81- 3.54 (m, 5H), 3.55-3.39 (m, 4H), 3.38-3.30 (s, 1H), 3.05- 2.95 (m, 1H), 2.88-2.78 (m, 1H), 2.73-2.63 (m, 1H), 2.62- 2.52 (m, 1H), 2.22-2.00 (m, 4H). |
| 259 | 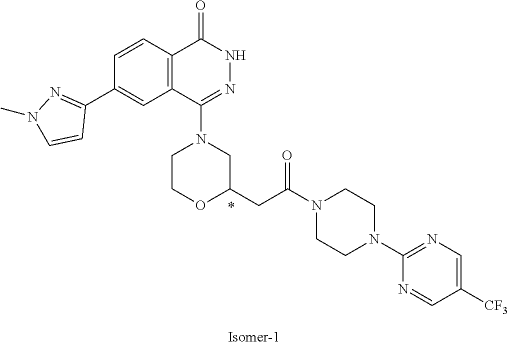 [SMILES: Cn1ccc(-c2ccc3c(=O)[nH]nc(N4CCOC(CC(=O)N5CCN(c6ncc(C(F)(F)F)cn6)CC5)C4)c3c2)n1] | 584.3 | ¹H NMR (400 MHz, methanol-d₄, ppm): δ 8.59 (s, 2H), 8.42-8.32 (m, 2H), 8.24 (dd, J = 8.4, 1.4 Hz, 1H), 7.69 (d, J = 2.2 Hz, 1H), 6.91 (d, J = 2.3 Hz, 1H), 4.38-4.28 (m, 1H), 4.11-3.80 (m, 9H), 3.79-3.58 (m, 5H), 3.43-3.33 (m, 1H), 3.10-2.99 (m, 1H), 2.92-2.74 (m, 2H), 2.65-2.55 (m, 1H). |
| 260 | 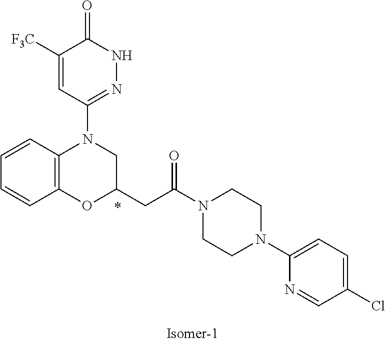 [SMILES: O=C(CC1CN(c2cc(C(F)(F)F)c(=O)[nH]n2)c2ccccc2O1)N1CCN(c2ccc(Cl)cn2)CC1] | 535.2 | SFC analysis: 100% ee; retention time: 4.407 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. 1.0FA salt, ¹H NMR (400 MHz, methanol-d₄, ppm): δ 8.51 (s, 1H), 8.07 (d, J = 2.6 Hz, 1H), 7.98 (s, 1H), 7.54 (dd, J = 9.1, 2.7 Hz, 1H), 7.08- 6.71 (m, 5H), 4.72-4.66 (m, 1H), 4.16 (dd, J = 13.2, 2.5 Hz, 1H), 3.85-3.44 (m, 9H), 3.07-2.97 (m, 1H), 2.84-2.74 (m, 1H). |
| 261 | 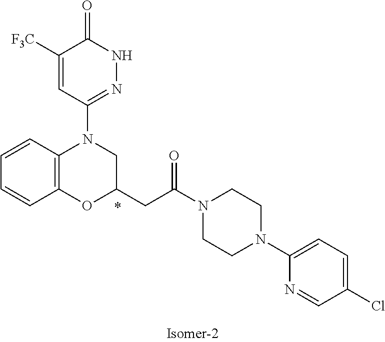 [SMILES: O=C(CC1CN(c2cc(C(F)(F)F)c(=O)[nH]n2)c2ccccc2O1)N1CCN(c2ccc(Cl)cn2)CC1] | 535.2 | SFC analysis: 100% ee; retention time: 4.935 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. 0.3FA salt, ¹H NMR (400 MHz, methanol-d₄, ppm): δ 8.52 (s, 0.32H), 8.07 (d, J = 2.6 Hz, 1H), 7.98 (s, 1H), 7.54 (dd, J = 9.1, 2.6 Hz, 1H), 7.09- 6.74 (m, 5H), 4.73-4.66 (dd, J = 11.0, 6.2 Hz, 1H), 4.16 (dd, J = 13.3, 2.6 Hz, 1H), 3.83-3.43 (m, 9H), 3.06-2.94 (m, 1H), 2.83-2.73 (m, 1H). |
| 262 | 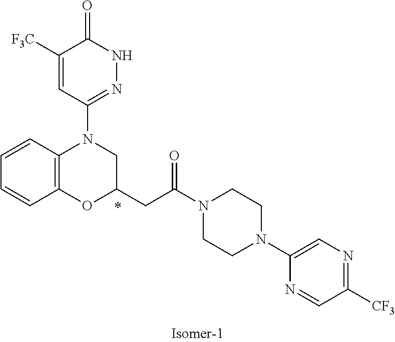 [SMILES: O=C(CC1CN(c2cc(C(F)(F)F)c(=O)[nH]n2)c2ccccc2O1)N1CCN(c2cnc(C(F)(F)F)cn2)CC1] | 570.2 | SFC analysis: 100% ee; retention time: 2.573 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.41 (s, 1H), 8.30 (s, 1H), 7.98 (s, 1H), 7.05-6.80 (m, 4H), 4.74-4.66 (m, 1H), 4.17 (dd, J = 13.4, 2.4 Hz, 1H), 3.91-3.69 (m, 8H), 3.53 (dd, J = 13.1, 7.6 Hz, 1H), 3.06- 2.97 (m, 1H), 2.84-2.75 (m, 1H). |
| 263 | 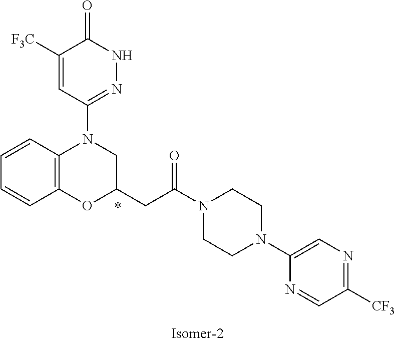 [SMILES: O=C(CC1CN(c2cc(C(F)(F)F)c(=O)[nH]n2)c2ccccc2O1)N1CCN(c2cnc(C(F)(F)F)cn2)CC1] | 570.2 | SFC analysis: 96.96% ee; retention time: 4.036 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.41 (s, 1H), 8.30 (s, 1H), 7.98 (s, 1H), 7.07-6.74 (m, 4H), 4.74-4.66 (m, 1H), 4.17 (dd, J = 13.2, 2.6 Hz, 1H), 3.91-3.67 (m, 8H), 3.53 (dd, J = 13.2, 7.7 Hz, 1H), 3.06-2..97 (m, 1H), 2.84-2.75 (m, 1H). |
| 264 | 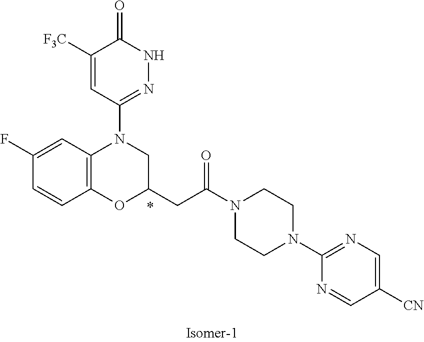 [SMILES: N#Cc1cnc(N2CCN(C(=O)CC3CN(c4cc(C(F)(F)F)c(=O)[nH]n4)c4cc(F)ccc4O3)CC2)nc1] | 545.2 | SFC analysis: 100% ee; retention time: 1.709 min; column: DAICEL CHIRALCEL ® AD, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.99 (s, 1H), 6.93-6.81 (m, 2H), 6.70- 6.59 (m, 1H), 4.69-4.61 (m, 1H), 4.10 (dd, J = 13.2, 2.5 Hz, 1H), 4.02-3.92 (m, 4H), 3.77-3.62 (m, 4H), 3.52 (dd, J = 13,1 7.6 Hz, 3.05-2.95 (m, 1H), 2.85-2.75 (m, 1H). |
| 265 | 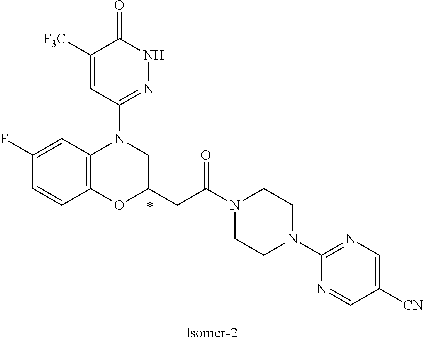 [SMILES: N#Cc1cnc(N2CCN(C(=O)CC3CN(c4cc(C(F)(F)F)c(=O)[nH]n4)c4cc(F)ccc4O3)CC2)nc1] | 545.2 | SFC analysis: 100% ee; retention time: 2.235 min; column: DAICEL CHIRALCEL ® AD, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.99 (s, 1H), 6.97-6.75 (m, 2H), 6.69- 6.58 (m, 1H), 4.69-4.61 (m, 1H), 4.10 (dd, J = 13.2, 2.5 Hz, 1H), 4.02-3.92 (m, 4H), 3.79-3.61 (m, 4H), 3.52 (dd, J = 13.1, 7.6 Hz, 1H), 3.04-2.95 (m, 1H), 2.86-2.76 (m, 1H). |
| 266 | 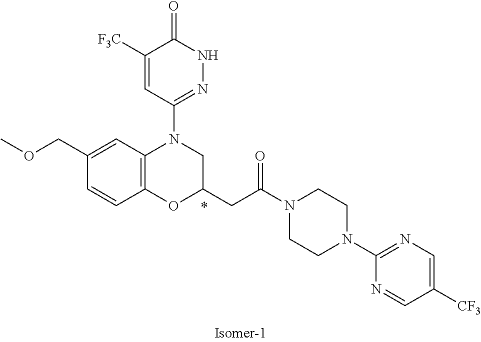 [SMILES: COCc1ccc2c(c1)N(c1cc(C(F)(F)F)c(=O)[nH]n1)CC(CC(=O)N1CCN(c3ncc(C(F)(F)F)cn3)CC1)O2] | 614.3 | SFC analysis: 99.38% ee; retention time: 1.765 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 8.00 (s, 1H), 6.99 (s, 1H), 6.93-6.87 (m, 2H), 4.73-4.64 (m, 1H), 4.33 (s, 2H), 4.20-4.11 (m, 1H), 4.02-3.89 (m, 4H), 3.77-3.62 (m, 4H), 3.55-3.49 (m, 1H), 3.32 (s, 3H), 3.05-2.96 (m, 1H), 2.84-2.75 (m, 1H). ¹⁹F NMR (376 MHz, methanol-d₄, ppm): δ −62.64 (3F), −68.68 (3F). |
| 267 | 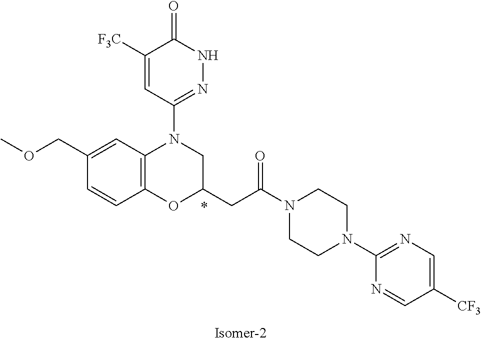 [SMILES: COCc1ccc2c(c1)N(c1cc(C(F)(F)F)c(=O)[nH]n1)CC(CC(=O)N1CCN(c3ncc(C(F)(F)F)cn3)CC1)O2] | 614.2 | SFC analysis: 99.02% ee; retention time: 2.503 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol d₄, ppm): δ 8.59 (s, 2H), 8.00 (s, 1H), 6.95-6.85 (m, 2H), 4.72- 4.63 (m, 1H), 4.33 (s, 2H), 4.20-4.11 (m, 1H), 4.02-3.88 (m, 4H), 3.78-3.62 (m, 4H), 3.57-3.47 (m, 1H), 3.32 (s, 3H), 3.06- 2.96 (m, 1H), 2.84-2.75 (m, 1H). ¹⁹F NMR (376 MHz, methanol-d₄, ppm): δ −62.64 (3F), −68.68 (3F). |
| 268 | 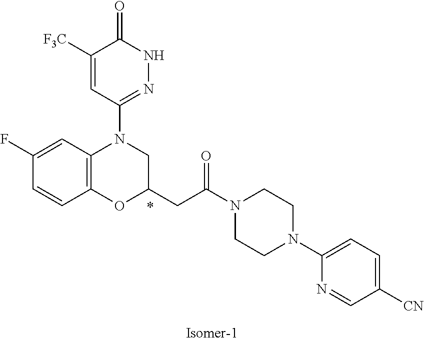 [SMILES: N#Cc1ccc(N2CCN(C(=O)CC3CN(c4cc(C(F)(F)F)c(=O)[nH]n4)c4cc(F)ccc4O3)CC2)nc1] | 544.2 | SFC analysis: 99.80% ee; retention time: 2.634 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.42 (s, 1H), 7.99 (s, 1H), 7.77 (dd, J = 9.0, 2.3 Hz, 1H), 6.90-6.80 (m, 3H), 6.70-6.60 (m, 1H), 4.69-4.59 (m, 1H), 4.15-4.06 (m, 1H), 3.84-3.63 (m, 8H), 3.56-3.47 (m, 1H), 3.05-2.94 (m, 1H), 2.83-2.74 (m, 1H). ¹⁹F NMR (376 MHz, methanol-d₄, ppm): δ −68.70 (3F), −123.49 (1F). |
| 269 | 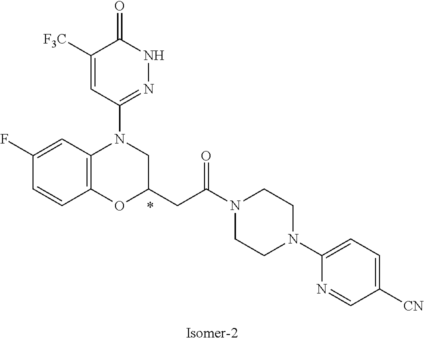 [SMILES: N#Cc1ccc(N2CCN(C(=O)CC3CN(c4cc(C(F)(F)F)c(=O)[nH]n4)c4cc(F)ccc4O3)CC2)nc1] | 544.2 | SFC analysis: 98.46% ee; retention time: 3.559 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.42 (s, 1H), 8.00 (s, 1H), 7.77 (d,d, J = 9.1, 2.3 Hz, 1H), 6.90-6.79 (m, 3H), 6.69-6.60 (m, 1H), 4.68-4.58 (m, 1H), 4.14-4.05 (m, 1H), 3.86-3.64 (m, 8H), 3.56-3.46 (m, 1H), 3.04-2.94 (m, 1H), 2.83-2.73 (m, 1H). ¹⁹F NMR (376 MHz, methanol-d₄, ppm): δ −68.69 (3F), −123.48 (1F). |
| 270 | 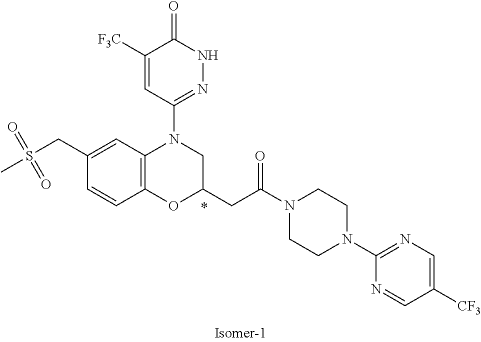 [SMILES: CS(=O)(=O)Cc1ccc2c(c1)N(c1cc(C(F)(F)F)c(=O)[nH]n1)CC(CC(=O)N1CCN(c3ncc(C(F)(F)F)cn3)CC1)O2] | 662.4 | SFC analysis: 99.66% ee; retention time: 3.028 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 8.04 (s, 1H), 7.09 (s, 1H), 7.04-6.96 (m, 1H), 6.97-6.88 (m, 1H), 4.76-4.67 (m, 1H), 4.29 (s, 2H), 4.22-4.13 (m, 1H), 4.03-.3.88 (m, 4H), 3.77-3.62 (m, 4H), 3.60-3.50 (m, 1H), 3.07-2.97 (m, 1H), 2.85 (s, 3H), 2.84- 2.75 (m, 1H). ¹⁹F NMR (376 MHz, methanol-d₄, ppm) δ −62.64 (3F), −68.48 (3F). |
| 271 | 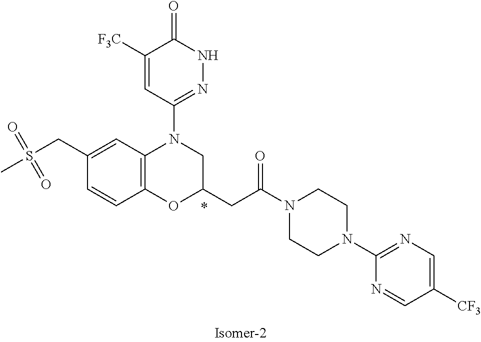 [SMILES: CS(=O)(=O)Cc1ccc2c(c1)N(c1cc(C(F)(F)F)c(=O)[nH]n1)CC(CC(=O)N1CCN(c3ncc(C(F)(F)F)cn3)CC1)O2] | 662.3 | SFC analysis: 99.08% ee; retention time: 4.144 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.59 (s, 2H), 8.04 (s, 1H), 7.09 (s, 1H), 7.04-6.87 (m, 2H), 4.78-4.63 (m, 1H), 4.29 (s, 2H), 4.22-4.10 (m, 1H), 4.04-3.83 (m, 4H), 3.78-3.61 (m, 4H), 3.60-3.49 (m, 1H), 3.08-2.93 (m, 1H), 2.85 (s, 3H), 2.84-2.74 (m, 1H). ¹⁹F NMR (376 MHz, methanol-d₄, ppm) δ −62.64 (3F), −68.49 (3F). |
| 273 | 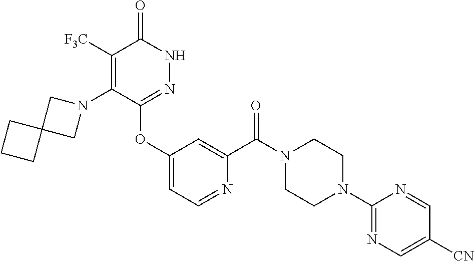 [SMILES: N#Cc1cnc(N2CCN(C(=O)c3cc(Oc4n[nH]c(=O)c(C(F)(F)F)c4N4CC5(CCC5)C4)ccn3)CC2)nc1] | 568.5 | 0.6TFA salt, ¹H NMR (400 MHz, DMSO-d₆, ppm): δ 11.83 (s, 1H), 8.80 (s, 2H), 8.62 (d, J = 5.6 Hz, 1H), 7.55 (s, 1H), 7.42 (d, J = 5.6, 1H), 4.37 (s, 4H), 4.04-3.94 (m, 2H), 3.93- 3.85 (m, 2H), 3.83-3.75 (m, 2H), 3.62-3.54 (m, 2H), 2.17- 2.08 (m, 4H), 1.77-1.68 (m, 2H). ¹⁹F NMR (376 MHz, DMSO-d₆, ppm): δ −51.37 (s, 3H). |
| 274 | 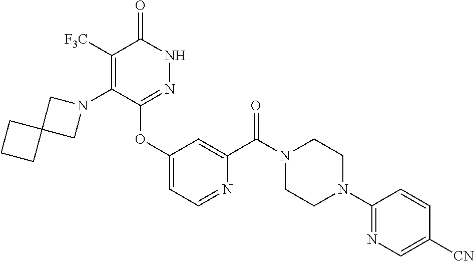 [SMILES: N#Cc1ccc(N2CCN(C(=O)c3cc(Oc4n[nH]c(=O)c(C(F)(F)F)c4N4CC5(CCC5)C4)ccn3)CC2)nc1] | 567.5 | 0.6TFA salt, ¹H NMR (400 MHz, DMSO-d₆, ppm): δ 11.82 (s, 1H), 8.62 (d, J = 5.7 Hz, 1H), 8.52 (s, 1H), 7.92 (d, J = 9.1 Hz, 1H), 7.55 (s, 1H), 7.42 (d, J = 5.6 Hz, 1H), 6.96 (d, J = 9.1 Hz, 1H), 4.37 (s, 4H), 3.87-3.65 (m, 6H), 3.64-3.55 (m, 2H), 2.20-2.05 (m, 4H), 1.77-1.66 (m, 2H). ¹⁹F NMR (376 MHz, DMSO-d₆, ppm): δ −51.37 (s, 3H). |
| 275 | 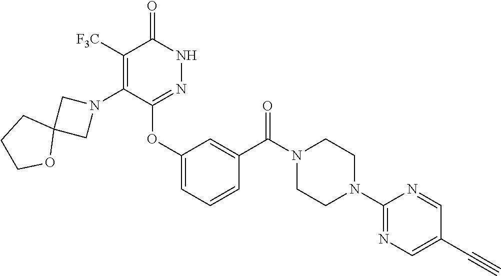 [SMILES: C#Cc1cnc(N2CCN(C(=O)c3cccc(Oc4n[nH]c(=O)c(C(F)(F)F)c4N4CC5(CCCO5)C4)c3)CC2)nc1] | 582.4 | ¹H NMR (400 MHz, DMSO-d₆, ppm): δ 11.63 (s, 1H), 8.51 (s, 2H), 7.51 (t, J = 8.0 Hz, 1H), 7.40-7.26 (m, 3H), 4.56- 4.46 (m, 2H), 4.44-4.35 (m, 2H), 4.32 (s, 1H), 3.95-3.79 (m, 4H), 3.78-3.64 (m, 4H), 3.50-3.38 (m, 2H), 2.11-2.02 (m, 2H), 1.88-1.78 (m, 2H). ¹⁹F NMR (376 MHz, DMSO-d₆, ppm): δ −51.27 (3F). |
| 276 | 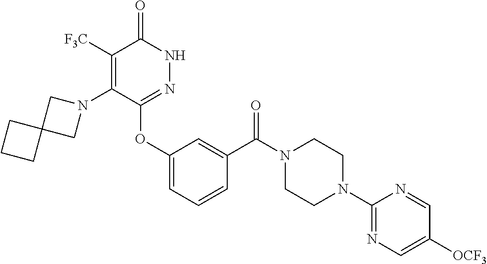 [SMILES: O=C(c1cccc(Oc2n[nH]c(=O)c(C(F)(F)F)c2N2CC3(CCC3)C2)c1)N1CCN(c2ncc(OC(F)(F)F)cn2)CC1] | 626.4 | ¹H NMR (400 MHz, DMSO-d₆, ppm): δ 11.55 (s, 1H), 8.55 (s, 2H), 7.57-7.49 (m, 1H), 7.39-7.27 (m, 3H), 4.43 (s, 4H), 3.91-3.63 (m, 6H), 3.58-3.37 (m, 2H), 2.20-2.10 (m, 4H), 1.79-1.68 (m, 2H). ¹⁹F NMR (376 MHz, DMSO-d₆, ppm): δ −51.25 (3F), −58.45 (3F). |
| 277 | 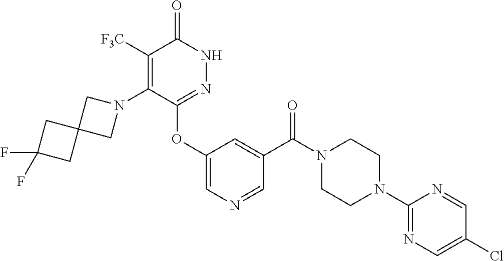 [SMILES: O=C(c1cncc(Oc2n[nH]c(=O)c(C(F)(F)F)c2N2CC3(C2)CC(F)(F)C3)c1)N1CCN(c2ncc(Cl)cn2)CC1] | 613.2 | ¹H NMR (400 MHz, DMSO-d₆, ppm): δ 11.66 (s, 1H), 8.70- 8.47 (m, 4H), 7.95-7.87 (m, 1H), 4.70-4.58 (m, 4H), 3.92- 3.65 (m, 6H), 3.51-3.38 (m, 2H), 2.89 (t, J = 12.5 Hz, 4H). ¹⁹F NMR (376 MHz, DMSO-d₆, ppm): δ −51.43 (3F), −91.01 (3F). |
| 278 | 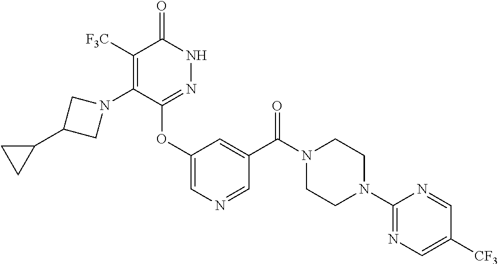 [SMILES: O=C(c1cncc(Oc2n[nH]c(=O)c(C(F)(F)F)c2N2CC(C3CC3)C2)c1)N1CCN(c2ncc(C(F)(F)F)cn2)CC1] | 611.4 | 1.0TFA salt, ¹H NMR (400 MHz, methanol-d₄, ppm): δ 8.68-8.53 (m, 4H), 8.00-7.91 (m, 1H), 4.74-4.64 (m, 2H), 4.39-4.29 (m, 2H), 4.15-3.95 (m, 4H), 3.92-3.84 (m, 2H), 3.62-3.51 (m, 2H), 2.31-2.26 (m, 1H), 1.18-1.04 (m, 1H), 0.60-0.49 (m, 2H), 0.21-0.17 (m, 2H). ¹⁹F NMR (376 MHz, methanol-d₄, ppm): δ −53.28 (3F), −62.66 (3F). |
| 279 | 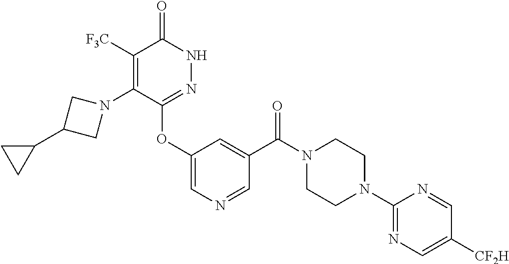 [SMILES: O=C(c1cncc(Oc2n[nH]c(=O)c(C(F)(F)F)c2N2CC(C3CC3)C2)c1)N1CCN(c2ncc(C(F)F)cn2)CC1] | 593.4 | 1.5TFA salt, ¹H NMR (400 MHz, methanol-d₄, ppm): δ 8.70-8.50 (m, 4H), 7.95-7.93 (m, 1H), 6.74 (t, J = 55.7 Hz, 1H), 4.75-4.65 (m, 2H), 4.39-4.31 (m, 2H), 4.12-3.79 (m, 6H), 3.62-3.51 (m, 2H), 2.33-2.24 (m, 1H), 1.18-1.02 (m, 1H), 0.61-0.48 (m, 2H), 0.21-0.17 (m, 2H). ¹⁹F NMR (376 MHz, methanol-d₄, ppm): δ −53.28 (F), −111.99 (3F). |
| 280 | 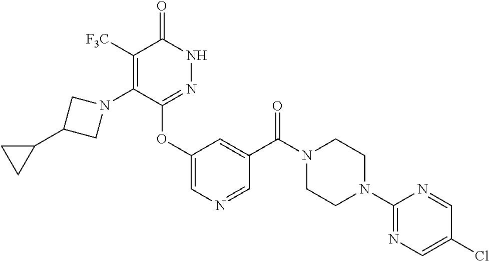 [SMILES: O=C(c1cncc(Oc2n[nH]c(=O)c(C(F)(F)F)c2N2CC(C3CC3)C2)c1)N1CCN(c2ncc(Cl)cn2)CC1] | 577.3 | 1.5TFA salt, ¹H NMR (400 MHz, methanol-d₄, ppm): δ 8.65-8.55 (m, 2H), 8.33 (s, 2H), 7.98-7.90 (m, 1H), 4.72- 4.62 (m, 2H), 4.39-4.29 (m, 2H), 4.02-3.78 (m, 6H), 3.62- 3.46 (m, 2H), 2.34-2.23 (m, 1H), 1.29-1.07 (m, 1H), 0.59- 0.50 (m, 2H), 0.20-0.18 (m, 2H). ¹⁹F NMR (376 MHz, methanol-d₄, ppm): δ −53.28 (3F). |
| 281 | 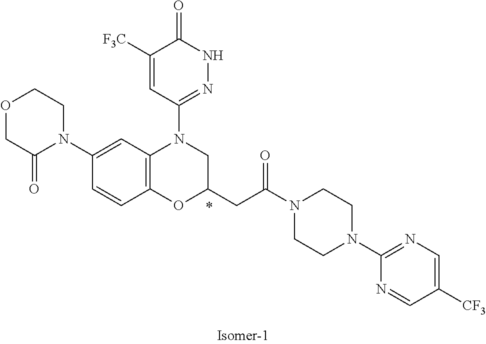 [SMILES: O=C(CC1CN(c2cc(C(F)(F)F)c(=O)[nH]n2)c2cc(N3CCOCC3=O)ccc2O1)N1CCN(c2ncc(C(F)(F)F)cn2)CC1] | 669.4 | SFC analysis: 100% ee; retention time: 2.878 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, DMSO-d₆, ppm) δ 13.07 (s, 1H), 8.74 (s, 2H), 7.99 (s, 1H), 7.13 (s, 1H), 6.93-6.85 (m, 2H), 4.70-4.60 (m, 1H), 4.14 (s, 2H), 4.10-4.03 (m, 1H), 3.95-3.77 (m, 6H), 3.69-3.55 (m, 6H), 3.52-3.44 (m, 1H), 2.98-2.90 (m, 1H), 2.80-2.75 (m, 1H). ¹⁹F NMR (376 MHz, DMSO-d₆, ppm) δ −59.29 (3F), −66.02 (3F). |
| 282 | 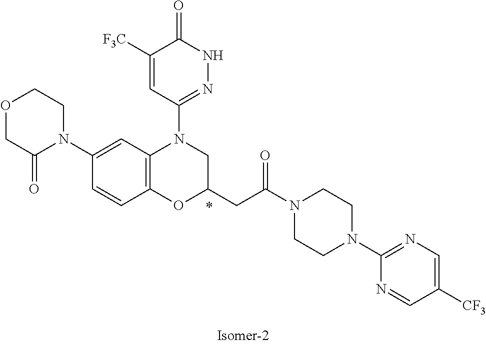 [SMILES: O=C(CC1CN(c2cc(C(F)(F)F)c(=O)[nH]n2)c2cc(N3CCOCC3=O)ccc2O1)N1CCN(c2ncc(C(F)(F)F)cn2)CC1] | 669.4 | SFC analysis: 90.62% ee; retention time: 4.276 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, DMSO-d₆, ppm) δ 13.09 (s, 1H), 8.74 (s, 2H), 7.99 (s, 1H), 7.13 (s, 1H), 6.94-6.85 (m, 2H), 4.70-4.60 (m, 1H), 4.14 (s, 2H), 4.09-4.01 (m, 1H), 3.95-3.75 (m, 6H), 3.69- 3.55 (m, 6H), 3.52-3.44 (m, 1H), 2.98-2.90 (m, 1H), 2.80- 2.75 (m, 1H). ¹⁹F NMR (376 MHz, DMSO-d₆, ppm) δ −59.29 (3F), −66.02 (3F). |
| 283 | 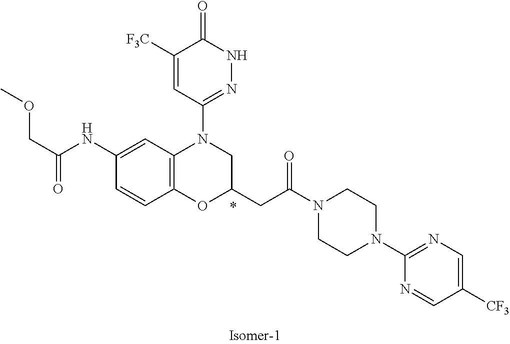 [SMILES: COCC(=O)Nc1ccc2c(c1)N(c1cc(C(F)(F)F)c(=O)[nH]n1)CC(CC(=O)N1CCN(c3ncc(C(F)(F)F)cn3)CC1)O2] | 657.4 | SFC analysis: 98.40% ee; retention time: 2.857 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 13.17 (s, 1H), 9.55 (s, 1H), 8.74 (s, 2H), 8.04 (s, 1H), 7.40 (d, J = 2.4 Hz, 1H), 7.18 (dd, J = 8.8, 2.4 Hz, 1H), 6.81 (d, J = 8.8 Hz, 1H), 4.65-4.58 (m, 1H), 4.02 (dd, J = 13.2, 2.4 Hz, 1H), 3.93 (s, 2H), 3.91-3.77 (m, 4H), 3.62- 3.54 (m, 4H), 3.45 (dd, J = 13.2, 7.6 Hz, 1H), 3.34 (s, 3H), 2.98-2.88 (m, 1H), 2.82-2.73 (m, 1H). ¹⁹F NMR (376 MHz, DMSO-d₆, ppm): δ −59.29 (3F), −65.99 (3F). |
| 284 | 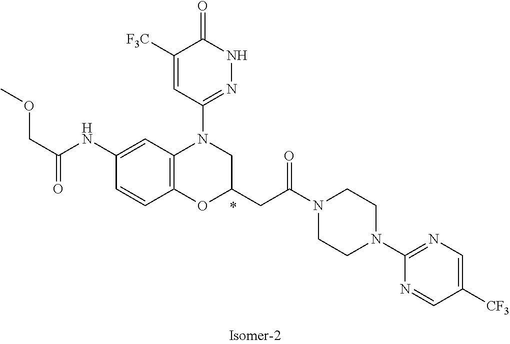 [SMILES: COCC(=O)Nc1ccc2c(c1)N(c1cc(C(F)(F)F)c(=O)[nH]n1)CC(CC(=O)N1CCN(c3ncc(C(F)(F)F)cn3)CC1)O2] | 657.4 | SFC analysis: 98.38% ee; retention time: 3.959 min; column: Regis WHELK-O1(S, S), 50 × 4.6 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 13.18 (s, 1H), 9.55 (s, 1H), 8.74 (s, 2H), 8.04 (s, 1H), 7.40 (d, J = 2.4 Hz, 1H), 7.18 (dd, J = 8.8, 2.4 Hz, 1H), 6.81 (d, J = 8.8 Hz, 1H), 4.65-4.58 (m, 1H), 4.02 (dd, J = 13.2, 2.4 Hz, 1H), 3.93 (s, 2H), 3.90-3.77 (m, 4H), 3.62- 3.54 (m, 4H), 3.45 (dd, J = 13.2, 7.6 Hz, 1H), 3.34 (s, 3H), 2.97-2.89 (m, 1H), 2.81-2.73 (m, 1H). ¹⁹F NMR (376 MHz, DMSO- d₆, ppm): δ −59.29 (3F), −65.99 (3F). |
| 285 | 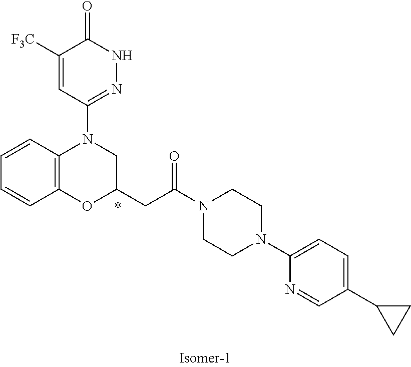 [SMILES: O=C(CC1CN(c2cc(C(F)(F)F)c(=O)[nH]n2)c2ccccc2O1)N1CCN(c2ccc(C3CC3)cn2)CC1] | 541.2 | SFC analysis: 100% ee; retention time: 2.547 min; column: DAICEL CHIRALCEL ® AS, 100 × 3.0 mm, 3 μm, A For CO₂ and B for MeOH (0.1% DEA), 40%; pressure: 100 bar; flow rate: 1.5 mL/min. ¹H NMR (400 MHz, methanol-d₄, ppm): δ 7.98 (s, 1H), 7.95 (d, J = 2.4 Hz, 1H), 7.29 (dd, J = 8.8, 2.5 Hz, 1H), 7.01-6.82 (m, 4H), 6.77 (d, J = 8.8 Hz, 1H), 4.73-4.65 (m, 1H), 4.16 (dd, J = 13.2, 2.6 Hz, 1H), 3.79-3.66 (m, 4H), 3.56-3.43 (m, 5H), 3.06-.294 (m, 1H), 2.84-2.75 (m, 1H), 1.88-1.76 (m, 1H), 0.96-0.87 (m, 2H), 0.64-0.52 (m, 2H). |
| 286 | 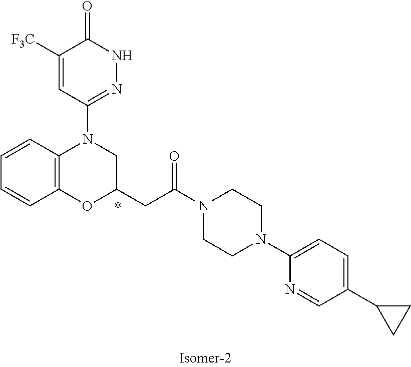 [SMILES: O=C(CC1CN(c2cc(C(F)(F)F)c(=O)[nH]n2)c2ccccc2O1)N1CCN(c2ccc(C3CC3)cn2)CC1] | 541.2 | SFC analysis: 100% ee; retention time: 2.109 min; column: DAICEL CHIRALCEL ® AS, 100 × 3.0 mm, 3 μm, A for CO₂ and B for MeOH (0.1% DEA), 40%; pressure: 100 bar; flow rate: 1.5 mL/min. 0.2FA salt, ¹H NMR (400 MHz, methanol-d₄, ppm): δ 8.39 (s, 0.21H), 7.98 (s, 1H), 7.95 (d, J = 2.4 Hz, 1H), 7.29 (dd, J = 8.8, 2.5 Hz, 1H), 7.02-6.82 (m, 4H), 6.77 (d, J = 8.8 Hz, 1H), 4.74-4.62 (m, 1H), 4.16 (dd, J = 13.2, 2.6 Hz, 1H), 3.77- 3.65 (m, 4H), 3.55-3.41 (m, 5H), 3.06- 2.95 (m, 1H), 2.84-2.74 (m, 1H), 1.88-1.77 (m, 1H), 0.97-0.87 (m, 2H), 0.65-0.54 (m, 2H). |
| 287 | 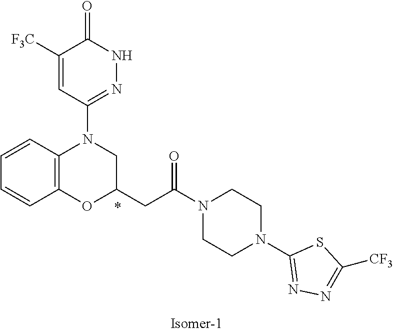 [SMILES: O=C(CC1CN(c2cc(C(F)(F)F)c(=O)[nH]n2)c2ccccc2O1)N1CCN(c2nnc(C(F)(F)F)s2)CC1] | 576.2 | SFC analysis: 99.60% ee; retention time 6.161 min; column: Regis WHELK-O1(S, S), 50 × 4.6 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 7.98 (s, 1H), 7.11-6.67 (m, 4H), 4.73- 4.65 (m, 1H), 4.17 (dd, J = 13.2, 2.6 Hz, 1H), 3.89-3.60 (m, 8H), 3.51 (dd, J = 13.2, 7.7 Hz, 1H), 3.06-2.97 (m, 1H), 2.84-2.75 (m, 1H). |
| 288 | 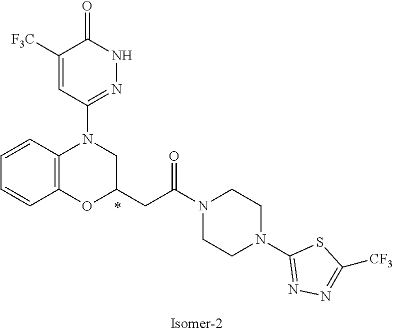 [SMILES: O=C(CC1CN(c2cc(C(F)(F)F)c(=O)[nH]n2)c2ccccc2O1)N1CCN(c2nnc(C(F)(F)F)s2)CC1] | 576.2 | SFC analysis: 99.16% ee; retention time: 7.055 min; column: Regis WHELK-O1(S, S), 50 × 4.6 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 7.98 (s, 1H), 7.18-6.67 (m, 4H), 4.73- 4.65 (m, 1H), 4.17 (dd, J = 13.2, 2.6 Hz, 1H), 3.90-3.58 (m, 8H), 3.51 (dd, J = 13.2, 7.7 Hz, 1H), 3.05-2.97 (m, 1H), 2.84-2.74 (m, 1H). |
| 289 | 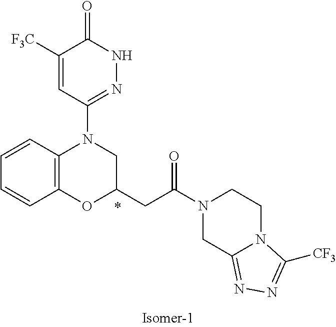 [SMILES: O=C(CC1CN(c2cc(C(F)(F)F)c(=O)[nH]n2)c2ccccc2O1)N1CCn2c(nnc2C(F)(F)F)C1] | 530.1 | SFC analysis: 99.36% ee; retention time: 4.593 min; column: DAICEL CHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 7.97 (s, 1H), 7.10-6.82 (m, 4H), 5.14-5.02 (m, 2H), 4.80-4.65 (m, 1H), 4.38-4.10 (m, 5H), 3.51 (dd, J = 13.1, 7.8 Hz, 1H), 3.18-3.02 (m, 1H), 2.96- 2.83 (m, 1H). |
| 290 | 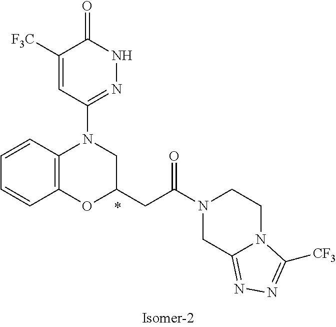 [SMILES: O=C(CC1CN(c2cc(C(F)(F)F)c(=O)[nH]n2)c2ccccc2O1)N1CCn2c(nnc2C(F)(F)F)C1] | 530.1 | SFC analysis: 98.00% ee; retention time: 3.779 min; column: DAICEL CHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 7.97 (s, 1H), 7.11-6.83 (m, 4H), 5.17-5.03 (m, 2H), 4.78-4.64 (m, 1H), 4.40-4.10 (m, 5H), 3.60-3.49 (m, 1H), 3.16-3.01 (m, 1H), 2.94-2.82 (m, 1H). |
| 291 | 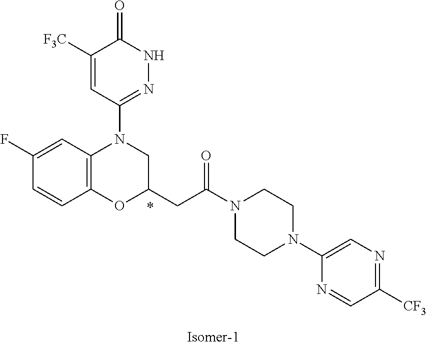 [SMILES: O=C(CC1CN(c2cc(C(F)(F)F)c(=O)[nH]n2)c2cc(F)ccc2O1)N1CCN(c2cnc(C(F)(F)F)cn2)CC1] | 588.2 | SFC analysis: 100% ee; retention time: 4.748 min; column: DAICEL CHIRALPAK ® IB, 100 × 3 mm, 3 μm, A for CO₂ and B for MeOH (0.1% DEA), 5% to 40%; pressure: 100 bar; flow rate: 1.5 mL/min. ¹H NMR (400 MHz, methanol- d₄, ppm): δ 8.41 (s, 1H), 8.30 (s, 1H), 7.99 (s, 1H), 6.92- 6.78 (m, 2H), 6.69-6.57 (m, 1H), 4.70-4.60 (m, 1H), 4.11 (dd, J = 13.1, 2.5 Hz, 1H), 3.88-3.68 (m, 8H), 3.52 (dd, J = 13.1, 7.7 Hz, 1H), 3.06-2.94 (m, 1H), 2.84-2.72 (m, 1H). |
| 292 | 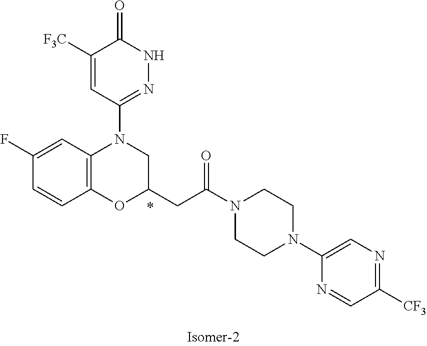 [SMILES: O=C(CC1CN(c2cc(C(F)(F)F)c(=O)[nH]n2)c2cc(F)ccc2O1)N1CCN(c2cnc(C(F)(F)F)cn2)CC1] | 588.2 | SFC analysis: 99.06% ee; retention time: 4.974 min; column: DAICEL CHIRALPAK ® IB, 100 × 3 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.41 (s, 1H), 8.30 (s, 1H), 7.99 (s, 1H), 6.92-6.79 (m, 2H), 6.70-6.61 (m, 1H), 4.70-4.60 (m, 1H), 4.11 (dd, J = 13.1, 2.5 Hz, 1H), 3.90-3.67 (m, 8H), 3.52 (dd, J = 13.1, 7.7 Hz, 1H), 3.05-2.96 (m, 1H), 2.84-2.75 (m, 1H). |
| 293 | 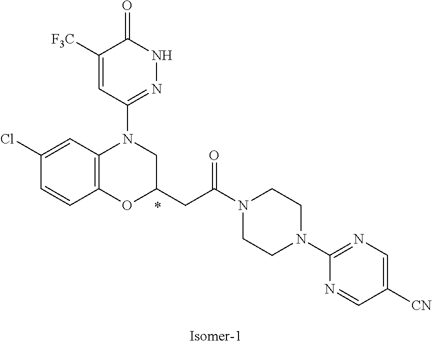 [SMILES: N#Cc1cnc(N2CCN(C(=O)CC3CN(c4cc(C(F)(F)F)c(=O)[nH]n4)c4cc(Cl)ccc4O3)CC2)nc1] | 561.2 | SFC analysi: 100% ee; retention time: 2.246 min; column: DAICEL CHIRALPAK ® AD, 100 × 3.0 mm, 3 μm, A for CO₂ and B for MeOH (0.1% DEA), 60%; pressure: 100 bar; flow rate: 1.5 mL/min. ¹H NMR (400 MHz, methanol-d₄, ppm): δ 8.63 (s, 2H), 7.98 (s, 1H), 7.08 (d, J = 2.0 Hz, 1H), 6.92-6.80 (m, 2H), 4.72-4.62 (m, 1H), 4.12-4.02 (m, 1H), 4.02-3.90 (m, 4H), 3.78-3.61 (m, 4H), 3.52 (dd, J = 13.2, 7.6 Hz, 1H), 3.06-2.96 (m, 1H), 2.84-2.74 (m, 1H). |
| 294 | 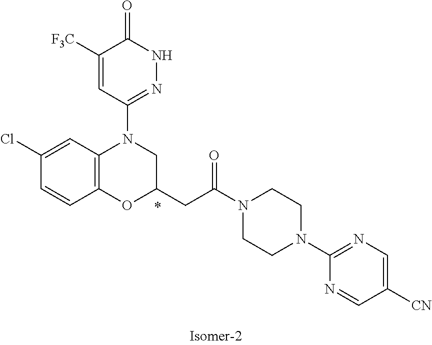 [SMILES: N#Cc1cnc(N2CCN(C(=O)CC3CN(c4cc(C(F)(F)F)c(=O)[nH]n4)c4cc(Cl)ccc4O3)CC2)nc1] | 561.2 | SFC analysis: 98.66% ee; retention time: 3.014 min; column: DAICEL CHIRALPAK ® AD, 100 × 3.0 mm, 3 μm, A for CO₂ and B for MeOH (0.1% DEA), 60%; pressure: 100 bar; flow rate: 1.5 mL/min. ¹H NMR (400 MHz, methanol-d₄, ppm): δ 8.63 (s, 2H), 7.98 (s, 1H), 7.08 (d, J = 2.1 Hz, 1H), 6.98-6.83 (m, 2H), 4.72-4.64 (m, 1H), 4.11-4.02 (m, 1H), 4.02-3.89 (m, 4H), 3.80-3.60 (m, 4H), 3.51 (dt, J = 15.5, 7.7 Hz, 1H), 3.05-2.96 (m, 1H), 2.84-2.74 (m, 1H). |
| 295 | 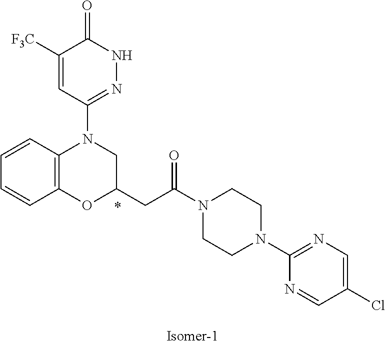 [SMILES: O=C(CC1CN(c2cc(C(F)(F)F)c(=O)[nH]n2)c2ccccc2O1)N1CCN(c2ncc(Cl)cn2)CC1] | 536.2 | SFC analysis: 100% ee; retention time: 4.035 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min, 1.0FA salt, ¹H NMR (400 MHz, methanl-d₄, ppm): δ 8.50 (brs, 1H), 8.32 (s, 2H), 7.98 (s, 1H), 7.05-6.78 (m, 4H), 4.74-4.65 (m, 1H), 4.16 (dd, J = 13.2, 2.6 Hz, 1H), 3.90-3.76 (m, 4H), 3.74-3.61 (m, 4H), 3.55-3.47 (m, 1H), 3.05-2.96 (m, 1H), 2.83-2.74 (m, 1H) |
| 296 | 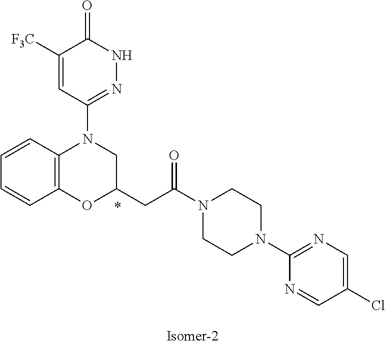 [SMILES: O=C(CC1CN(c2cc(C(F)(F)F)c(=O)[nH]n2)c2ccccc2O1)N1CCN(c2ncc(Cl)cn2)CC1] | 536.2 | SFC analysis: 100% ee; retention time: 4.519 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. 1.0FA salt, ¹H NMR (400 MHz, methanol-d₄, ppm): δ 8.51 (brs, 1H), 8.32 (s, 2H), 7.98 (s, 1H), 7.05-6.78 (m, 4H), 4.74-4.65 (m, 1H), 4.16 (dd, J = 13.2, 2.3 Hz, 1H), 3.91-3.78 (m, 4H), 3.76-3.57 (m, 4H), 3.56-3.48 (m, 1H), 3.05-2.96 (m, 1H), 2.84-2.74 (m, 1H). |
| 297 | 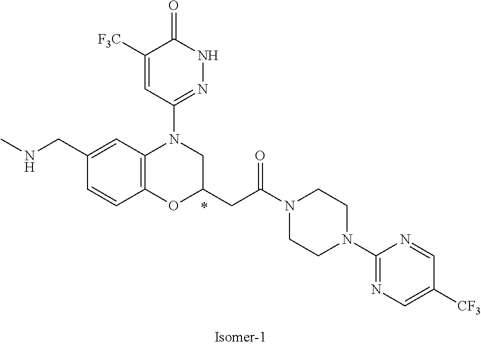 [SMILES: CNCc1ccc2c(c1)N(c1cc(C(F)(F)F)c(=O)[nH]n1)CC(CC(=O)N1CCN(c3ncc(C(F)(F)F)cn3)CC1)O2] | 613.4 | SFC analysis: 100% ee; retention time: 1.651 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, DMSO-d₆, ppm): δ 8.75 (s, 2H), 8.00 (s, 1H), 7.10 (s, 1H), 6.99-6.88 (m, 2H), 4.71-4.60 (m, 1H), 4.10-4.00 (m, 1H), 3.96-3.73 (m, 6H), 3.66-3.53 (m, 4H), 3.49-3.41 (m, 1H), 3.00-2.88 (m, 1H), 2.84-2.74 (m, 1H), 2.42 (s, 3H). ¹⁹F NMR (376 MHz, DMSO-d₆, ppm): δ −59.28 (3F), −66.00 (3F). |
| 298 | 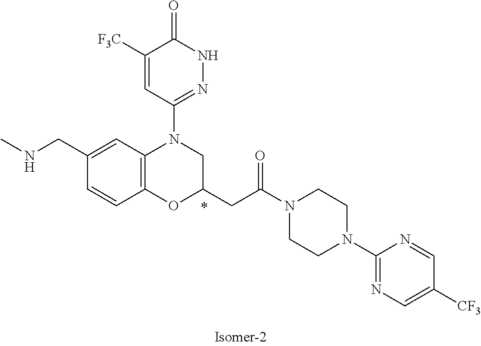 [SMILES: CNCc1ccc2c(c1)N(c1cc(C(F)(F)F)c(=O)[nH]n1)CC(CC(=O)N1CCN(c3ncc(C(F)(F)F)cn3)CC1)O2] | 613.4 | SFC analysis: 97.16% ee; retention time: 2.348 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. 1.6TFA salt, ¹H NMR (400 MHz, DMSO-d₆, ppm): δ 8.75 (s, 2H), 8.00 (s, 1H), 7.13 (s, 1H), 7.00-6.90 (m, 2H), 4.71-4.60 (m, 1H), 4.10-4.00 (m, 1H), 3.99-3.73 (m, 6H), 3.68-3.53 (m, 4H), 3.49-3.40 (m, 1H), 3.00-2.88 (m, 1H), 2.84-2.74 (m, 1H), 2.44 (s, 3H); ¹⁹F NMR (376 MHz, DMSO-d₆, ppm): δ −59.27 (3F), −65.99 (3F). |
| 299 | 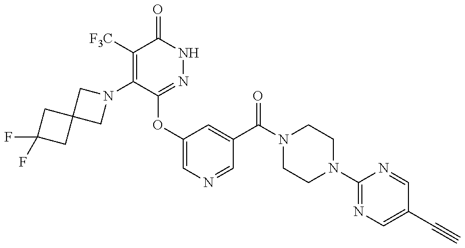 [SMILES: C#Cc1cnc(N2CCN(C(=O)c3cncc(Oc4n[nH]c(=O)c(C(F)(F)F)c4N4CC5(C4)CC(F)(F)C5)c3)CC2)nc1] | 603.4 | ¹H NMR (400 MHz, methanol-d₄, ppm): δ 8.62 (d, J = 2.6 Hz, 1H), 8.56 (d, J = 1.7 Hz, 1H), 8.43 (s, 2H), 7.91 (dd, J = 2.6, 1.7 Hz, 1H), 4.69 (s, 4H), 4.07-3.78 (m, 6H), 3.64 (s, 1H), 3.61-3.51 (m, 2H), 2.85 (t, J = 12.1 Hz, 4H). ¹⁹F NMR (376 MHz, methanol-d₄, ppm): δ −53.43 (3F), −94.71 (2F) |
| 300 | 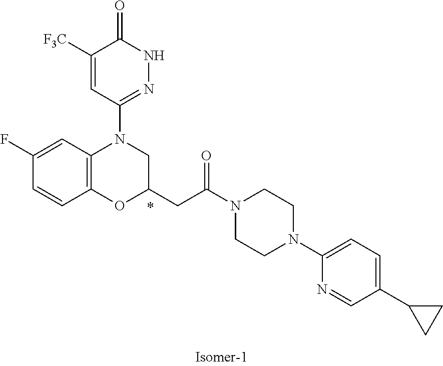 [SMILES: O=C(CC1CN(c2cc(C(F)(F)F)c(=O)[nH]n2)c2cc(F)ccc2O1)N1CCN(c2ccc(C3CC3)cn2)CC1] | 559.3 | SFC analysis: 100% ee; retention time: 2.236 min; column: DAICEL CHIRALPAK ® AD, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₆, ppm): δ 7.99 (s, 1H), 7.95 (d, J = 2.4 Hz, 1H), 7.29 (dd, J = 8.7, 2.5 Hz, 1H), 6.95-6.81 (m, 2H), 6.77 (d, J = 8.8 Hz, 1H), 6.69-6.58 (m, 1H), 4.68-4.59 (m, 1H), 4.10 (dd, J = 13.1, 2.5 Hz, 1H), 3.79-3.62 (m, 4H), 3.58-3.40 (m, 5H), 3.05-2.94 (m, 1H), 2.82-2.73 (m, 1H), 1.88-1.77 (m, 1H), 0.96-0.86 (m, 2H), 0.63-0.57 (m, 2H). |
| 301 | 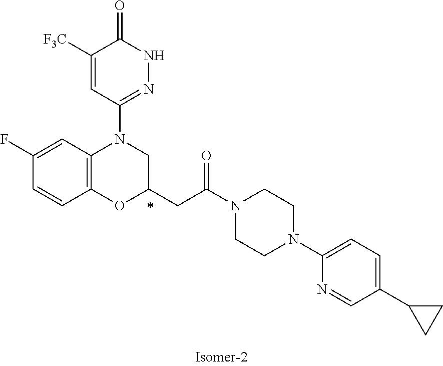 [SMILES: O=C(CC1CN(c2cc(C(F)(F)F)c(=O)[nH]n2)c2cc(F)ccc2O1)N1CCN(c2ccc(C3CC3)cn2)CC1] | 559.2 | SFC analysis: 100% ee; retention time: 2.795 min; column: DAICEL CHIRALPAK ® AD, 100 × 3.0 mm, 3 μm, A for CO₂ and B for MeOH (0.1% DEA), 40%; pressure: 100 bar; flow rate: 1.5 mL/min. 0.2FA salt, ¹H NMR (400 MHz, methanol-d₄, ppm): δ 8.49 (brs, 0.2H), 7.99 (s, 1H), 7.95 (d, J = 2.4 Hz, 1H), 7.29 (dd, J = 8.8, 2.4 Hz, 1H), 6.92-6.80 (m, 2H), 6.77 (d, J = 8.8 Hz, 1H), 6.71-6.58 (m, 1H), 4.69- 4.60 (m, 1H), 4.10 (dd, J = 13.1, 2.5 Hz, 1H), 3.80-3.63 (m, 4H), 3.58-3.40 (m, 5H), 3.05- 2.94 (m, 1H), 2.82-2.72 (m, 1H), 1.87-1.77 (m, 1H), 0.96- 0.87 (m, 2H), 0.62-0.56 (m, 2H). |
| 302 | 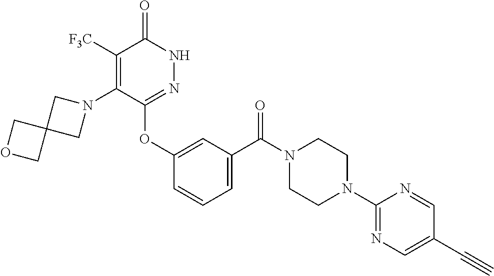 [SMILES: C#Cc1cnc(N2CCN(C(=O)c3cccc(Oc4n[nH]c(=O)c(C(F)(F)F)c4N4CC5(COC5)C4)c3)CC2)nc1] | 568.2 | ¹H NMR (400 MHz, DMSO-d₆, ppm): δ 11.60 (s, 1H), 8.51 s, 2H), 7.54-7.50 (m, 1H), 7.34- 7.29 (m, 3H), 4.71-4.58 (m, 8H), 4.31 (s, 1H), 3.89-3.64 (m, 6H), 3.48-3.38 (m, 2H). ¹⁹F NMR (376 MHz, DMSO-d₆, ppm): δ −51.47 (3F) |
| 303 | 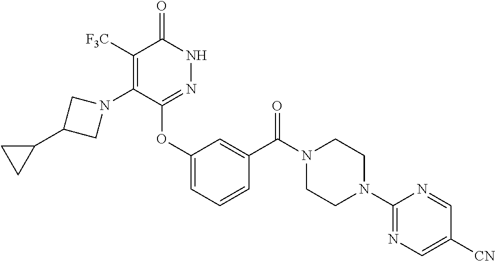 [SMILES: N#Cc1cnc(N2CCN(C(=O)c3cccc(Oc4n[nH]c(=O)c(C(F)(F)F)c4N4CC(C5CC5)C4)c3)CC2)nc1] | 567.5 | ¹H NMR (400 MHz, DMSO-d₆, ppm): δ 11.59 (s, 1H), 8.80 (s, 2H), 7.58-7.47 (m, 1H), 7.40- 7.24 (m, 3H), 4.60-4.44 (m, 2H), 4.21-4.06 (m, 2H), 4.05-3.81 (m, 4H), 3.79-3.40 (m, 4H), 2.30-2.18 (m, 1H), 1.16-1.03 (m, 1H), 0.49-0.38 (m, 2H), 0.19-0.08 (m, 2H). ¹⁹F NMR (376 MHz, DMSO-d₆, ppm): δ −49.01 (3F). |
| 304 | 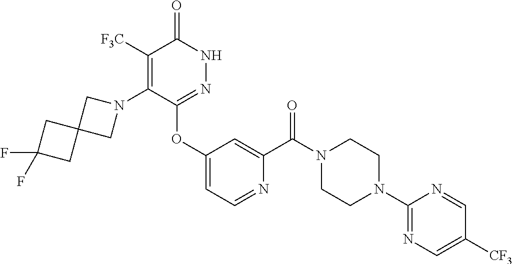 [SMILES: O=C(c1cc(Oc2n[nH]c(=O)c(C(F)(F)F)c2N2CC3(C2)CC(F)(F)C3)ccn1)N1CCN(c2ncc(C(F)(F)F)cn2)CC1] | 647.2 | 0.3TFA salt, ¹H NMR (400 MHz, DMSO-d₆, ppm): δ 11.89 (s, 1H), 8.75 (s, 2H), 8.63 (d, J = 5.6 Hz, 1H), 7.55 (s, 1H), 7.41 (d, J = 5.6 Hz, 1H), 4.51 (s, 4H), 4.00-3.81 (m, 4H), 3.80-3.69 (m, 2H), 3.65-3.55 (m, 2H), 2.85 (t, J = 12.5 Hz, 4H). ¹⁹F NMR (376 MHz, DMSO- d₆, ppm): δ −51.54 (3F), 59.29 (3F), −91.06 (2F). |
| 305 | 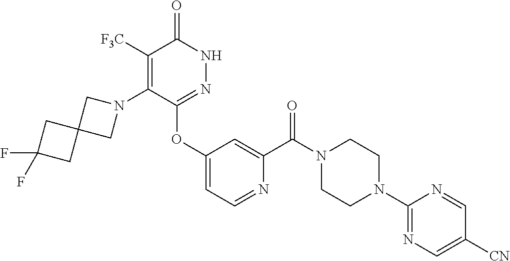 [SMILES: N#Cc1cnc(N2CCN(C(=O)c3cc(Oc4n[nH]c(=O)c(C(F)(F)F)c4N4CC5(C4)CC(F)(F)C5)ccn3)CC2)nc1] | 604.4 | 0.7TFA salt, ¹H NMR (400 MHz, DMSO-d₆, ppm): δ 11.89 (s, 1H), 8.80 (s, 2H), 8.62 (d, J = 5.6 Hz, 1H), 7.55 (s, 1H), 7.41 (d, J = 5.6 Hz, 1H), 4.51 (s, 4H), 4.02-3.93 (m, 2H), 3.92-3.83 (m, 2H), 3.82-3.71 (m, 2H), 3.64-3.54 (m, 2H), 2.85 (t, J = 12.5 Hz, 4H). ¹⁹F NMR (376 MHz, DMSO-d₆, ppm): δ −51.54 (3F), −91.05 (2F). |
| 306 | 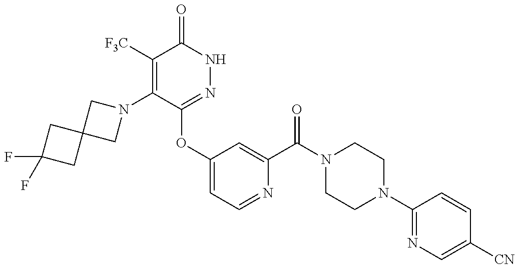 [SMILES: N#Cc1ccc(N2CCN(C(=O)c3cc(Oc4n[nH]c(=O)c(C(F)(F)F)c4N4CC5(C4)CC(F)(F)C5)ccn3)CC2)nc1] | 603.4 | 1.2TFA salt, ¹H NMR (400 MHz, DMSO-d₆, ppm): δ 11.87 (s, 1H), 8.62 (d, J = 5.7 Hz, 1H), 8.52 (s, 1H), 7.92 (d, J = 9.1 Hz, 1H), 7.54 (s, 1H), 7.41 (d, J = 5.6 Hz, 1H), 6.96 (d, J = 9.1 Hz, 1H), 4.50 (s, 4H), 3.87-3.79 (m, 2H), 3.78-3.67 (m, 4H), 3.65-3.55 (m, 2H), 2.85 (t, J = 12.5 Hz, 4H). ¹⁹F NMR (376 MHz, DMSO-d₆, ppm): δ −51.55 (3F), −91.05 (2F). |
| 307 | 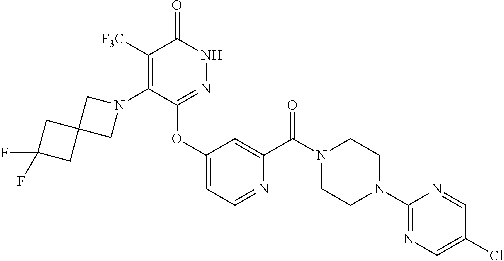 [SMILES: O=C(c1cc(Oc2n[nH]c(=O)c(C(F)(F)F)c2N2CC3(C2)CC(F)(F)C3)ccn1)N1CCN(c2ncc(Cl)cn2)CC1] | 613.2 | 0.4TFA salt, ¹H NMR (400 MHz, DMSO-d₆, ppm): δ 11.88 (s, 1H), 8.62 (d, J = 5.6 Hz, 1H), 8.46 (s, 2H), 7.54 (s, 1H), 7.41 (d, J = 5.6 Hz, 1H), 4.51 (s, 4H), 3.90-3.81 (m, 2H), 3.80-3.68 (m, 4H), 3.60-3.46 (m, 2H), 2.85 (t, J = 12.5 Hz, 4H). ¹⁹F NMR (376 MHz, DMSO-d₆, ppm): δ −51.55 (3F), −91.06 (2F). |
| 308 | 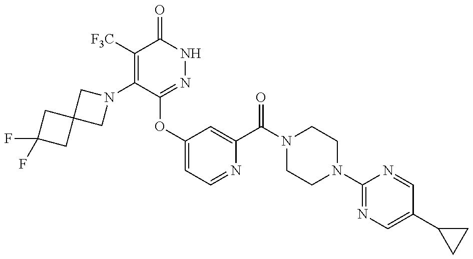 [SMILES: O=C(c1cc(Oc2n[nH]c(=O)c(C(F)(F)F)c2N2CC3(C2)CC(F)(F)C3)ccn1)N1CCN(c2ncc(C3CC3)cn2)CC1] | 619.4 | 1.4TFA salt, ¹H NMR (400 MHz, DMSO-d₆, ppm): δ 11.88 (s, 1H), 8.62 (d, J = 5.6 Hz, 1H), 8.20 (s, 2H), 7.53 (s, 1H), 7.40 (d, J = 5.7 Hz, 1H), 4.50 (s, 4H), 3.85-3.75 (m, 2H), 3.74-3.63 (m, 4H), 3.60-3.49 (m, 2H), 2.85 (t, J = 12.6 Hz, 4H), 1.84-1.73 (m, 1H), 0.93-0.82 (m, 2H), 0.69-0.58 (m, 2H). ¹⁹F NMR (376 MHz, DMSO-d₆, ppm): δ −51.55 (3F), −91.06 (2F). |
| 309 | 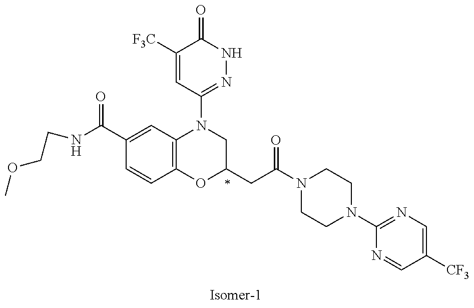 [SMILES: COCCNC(=O)c1ccc2c(c1)N(c1cc(C(F)(F)F)c(=O)[nH]n1)CC(CC(=O)N1CCN(c3ncc(C(F)(F)F)cn3)CC1)O2] | 671.3 | SFC analysis: 100% ee; retention time: 4.657 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.60 (s, 2H), 8.01 (s, 1H), 7.53 (d, J = 2.0 Hz, 1H), 7.44 (dd, J = 8.5, 2.0 Hz, 1H), 6.96 (d, J = 8.5 Hz, 1H), 4.8-4.73 (m, 1H), 4.15 (dd, J = 13.2, 2.5 Hz, 1H), 4.02-3.89 (m, 4H), 3.78-3.61 (m, 4H), 3.60-3.47 (m, 5H), 3.35 (s, 3H), 3.09-3.00 (m, 1H), 2.87-2.77 (m, 1H). |
| 310 | 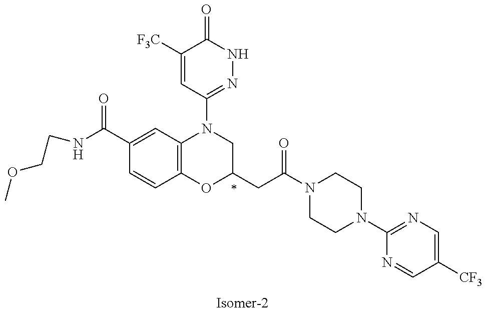 [SMILES: COCCNC(=O)c1ccc2c(c1)N(c1cc(C(F)(F)F)c(=O)[nH]n1)CC(CC(=O)N1CCN(c3ncc(C(F)(F)F)cn3)CC1)O2] | 671.2 | SFC analysis: 100% ee; retention time: 3.945 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.60 (s, 2H), 8.01 (s, 1H), 7.53 (d, J = 2.0 Hz, 1H), 7.44 (dd, J = 8.4, 2.0 Hz, 1H), 6.96 (d, J = 8.6 Hz, 1H), 4.84-4.71 (m, 1H), 4.15 (dd, J = 13.3, 2.6 Hz, 1H), 4.02-3.90 (m, 4H), 3.60-3.48 (m, 4H), 3.59-3.47 (m, 5H), 3.35 (s, 3H), 3.09-3.00 (m, 1H), 2.88-2.78 (m, 1H). |
| 311 | 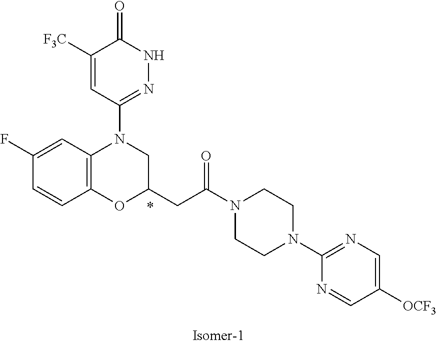 [SMILES: O=C(CC1CN(c2cc(C(F)(F)F)c(=O)[nH]n2)c2cc(F)ccc2O1)N1CCN(c2ncc(OC(F)(F)F)cn2)CC1] | 604.2 | SFC analysis: 100% ee; retention time: 4.229 min; column: DAICEL CHIPAKCEL ® OZ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.36 (s, 2H), 7.99 (s, 1H), 6.89-6.82 (m, 2H), 6.70-6.61 (m, 1H), 4.69-4.60 (m, 1H), 4.10 (dd, J = 13.1, 2.5 Hz, 1H), 3.93-3.81 (m, 4H), 3.76-3.59 (m, 4H), 3.52 (dd, J = 13.1, 7.6 Hz, 1H), 3.07- 2.94 (m, 1H), 2.85- 2.71 (m, 1H). |
| 312 | 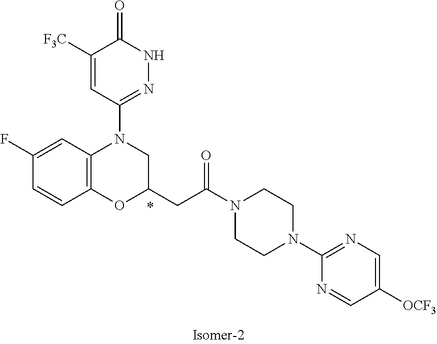 [SMILES: O=C(CC1CN(c2cc(C(F)(F)F)c(=O)[nH]n2)c2cc(F)ccc2O1)N1CCN(c2ncc(OC(F)(F)F)cn2)CC1] | 604.2 | SFC analysis: 100% ee; retention time: 4.488 min; column: DAICEL CHIPAKCEL ® OZ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.36 (s, 2H), 7.99 (s, 1H), 6.90-6.79 (m, 2H), 6.70-6.60 (m, 1H), 4.70- 4.60 (m, 1H), 4.10 (dd, J = 13.1, 2.5 Hz, 1H), 3.94-3.80 (m, 4H), 3.76-3.61 (m, 4H), 3.52 (dd, J = 13.1, 7.6 Hz, 1H), 3.07-2.95 (m, 1H), 2.83- 2.73 (m, 1H). |
| 313 | 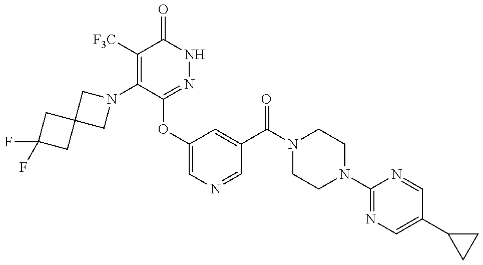 [SMILES: O=C(c1cncc(Oc2n[nH]c(=O)c(C(F)(F)F)c2N2CC3(C2)CC(F)(F)C3)c1)N1CCN(c2ncc(C3CC3)cn2)CC1] | 619.4 | 0.9TFA salt, ¹H NMR (400 MHz, methanol-d₄, ppm): δ 8.63 (s, 1H), 8.56 (s, 1H), 8.22 (s, 2H), 7.91 (d, J = 2.6 Hz, 1H), 4.69 (s, 4H), 3.97-3.77 (m, 6H), 3.66-3.51 (m, 2H), 2.85 (t, J = 12.1 Hz, 4H), 1.88-1.78 (m, 1H), 1.01-0.91 (m, 2H), 0.,71-0.61 (m, 2H). ¹⁹F NMR (376 MHz, methanol-d₄, ppm): δ −53.43 (3F), −94.71 (2F). |
| 314 | 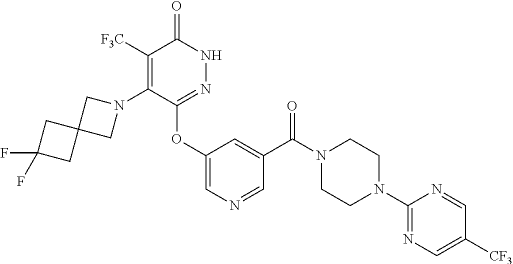 [SMILES: O=C(c1cncc(Oc2n[nH]c(=O)c(C(F)(F)F)c2N2CC3(C2)CC(F)(F)C3)c1)N1CCN(c2ncc(C(F)(F)F)cn2)CC1] | 661.4 | 0.6TFA salt, ¹H NMR (400 MHz, methanol-d₄, ppm): δ 8.64-8.55 (m, 4H), 7.99-7.88 (m, 1H), 5.22-5.00 (m, 1H), 4.81-4.65 (m, 5H), 4.63-4.45 (m, 1H), 3.88-3.74 (m, 1H), 3.68-3.54 (m, 1H), 3.49-3.33 (m, 1H), 3.25-3.09 (m, 1H), 2.85 (t, J = 12.1 Hz, 4H), 1.37-1.17 (m, 3H). ¹⁹F NMR (376 MHz, methanol-d₄, ppm): δ −53.43 (3F), δ −62.65 (3F), −94.71 (2F). |
| 315 | 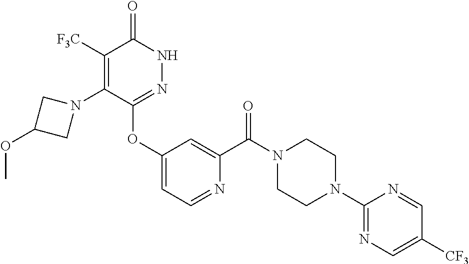 [SMILES: COC1CN(c2c(Oc3ccnc(C(=O)N4CCN(c5ncc(C(F)(F)F)cn5)CC4)c3)n[nH]c(=O)c2C(F)(F)F)C1] | 601.3 | 1.0TFA salt, ¹H NMR (400 MHz, methanol-d₄, ppm): 8.68-8.56 (m, 3H), 7.57 (s, 1H), 7.41-7.35 (m, 1H), 4.73- 4.63 (m, 2H), 4.40-4.30 (m, 2H), 4.26-4.17 (m, 1H), 4.13- 4.03 (m, 2H), 4.02-3.93 (m, 2H), 3.92-3.80 (m, 2H), 3.67- 3.56 (m, 2H), 3.31 (s, 3H). ¹⁹F NMR (376 MHz, methanol- d₄, ppm): δ −53.40 (3F), −62.65 (3F). |
| 316 | 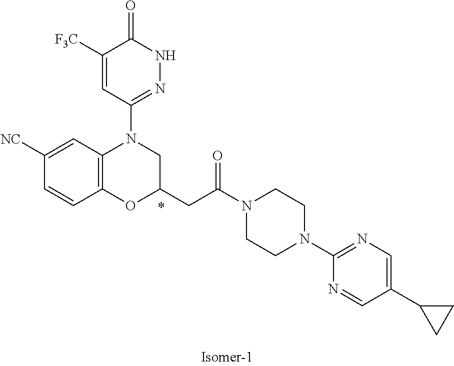 [SMILES: N#Cc1ccc2c(c1)N(c1cc(C(F)(F)F)c(=O)[nH]n1)CC(CC(=O)N1CCN(c3ncc(C4CC4)cn3)CC1)O2] | 567.3 | SFC analysis: 100% ee; retention time: 4.312 min; column: DAICEL CHIRALPAK ® IA, 250 × 4.6 mm, 5 μm, A for CO₂ and B for MeOH (0.1% DEA), 40%; pressure: 100 bar; flow rate: 1.5 mL/min. ¹H NMR (400 MHz, methanol-d₄, ppm): δ 8.16 (s, 2H), 7.99 (s, 1H), 7.51 (d, J = 1.8 Hz, 1H), 7.26 (dd, J = 8.4, 1.8 Hz, 1H), 7.02 (d, J = 8.4 Hz, 1H), 4.79-4.73 (m, 1H), 4.61-4.55 (m, 1H), 3.85-3.73 (m, 4H), 3.70-3.60 (m, 4H), 3.56 (dd, J = 13.3, 7.6 Hz, 1H), 3.09-2.97 (m, 1H), 2.88-2.76 (m, 1H), 1.87-1.62 (m, 1H), 1.01-0.85 (m, 2H), 0.70-0.55 (m, 2H). |
| 317 | 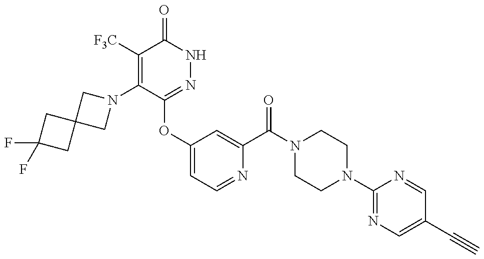 [SMILES: C#Cc1cnc(N2CCN(C(=O)c3cc(Oc4n[nH]c(=O)c(C(F)(F)F)c4N4CC5(C4)CC(F)(F)C5)ccn3)CC2)nc1] | 603.2 | ¹H NMR (400 MHz, DMSO-d₆, ppm): δ 11.88 (s, 1H), 8.62 (d, J = 5.6 Hz, 1H), 8.51 (s, 2H), 7.54 (s, 1H), 7.40 (d, J = 5.6 Hz, 1H), 4.51 (s, 4H), 4.32 (s, 1H), 3.95-3.84 (m, 2H), 3.83-3.64 (m, 4H), 3.60-3.47 (m, 2H), 2.95-2.77 (m, 4H). ¹⁹F NMR (376 MHz, DMSO-d₆, ppm): δ −51.54 (3F), −91.06 (2F). |
| 318 | 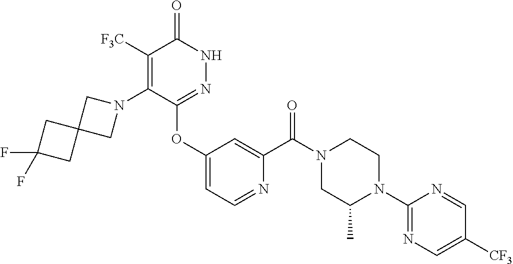 [SMILES: C[C@@H]1CN(C(=O)c2cc(Oc3n[nH]c(=O)c(C(F)(F)F)c3N3CC4(C3)CC(F)(F)C4)ccn2)CCN1c1ncc(C(F)(F)F)cn1] | 661.4 | 0.4TFA salt, ¹H NMR (400 MHz, DMSO-d₆, ppm): δ 11.90 (d, J = 10.2 Hz, 1H), 8.75 (s, 2H), 8.68-8.58 (m, 1H), 7.58 (d, J = 14.2 Hz, 1H), 7.46-7.36 (m, 1H), 5.13-4.79 (m, 1H), 4.66-4.56 (m, 1H), 4.55-4.36 (m, 5H), 3.94-3.80 (m, 1H), 3.74-3.46 (m, 1H), 3.38-3.04 (m, 2H), 2.95-2.77 (m, 4H), 1.22-1.08 (m, 3H). ¹⁹F NMR (376 MHz, DMSO-d₆, ppm): δ −51.54 (3F), −59.30 (3F), −91.06 (2F). |
| 319 | 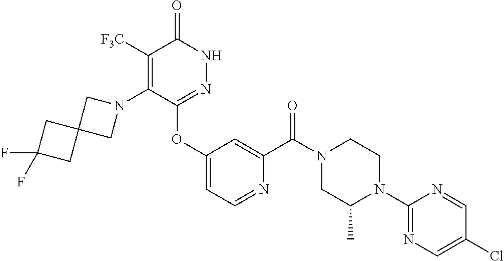 [SMILES: C[C@@H]1CN(C(=O)c2cc(Oc3n[nH]c(=O)c(C(F)(F)F)c3N3CC4(C3)CC(F)(F)C4)ccn2)CCN1c1ncc(Cl)cn1] | 627.3 | 4.2TFA salt, ¹H NMR (400 MHz, DMSO-d₆, ppm): δ 11.91 (s, 1H), 8.64-8.54 (m, 1H), 8.47 (s, 2H), 7.60-7.48 (m, 1H), 7.46-7.34 (m, 1H), 4.99-4.62 (m, 1H), 4.51-4.32 (m, 6H), 3.90-3.60 (m, 1H), 3.29-2.98 (m, 3H), 2.95-2.76 (m, 4H), 1.16-1.02 (m, 3H); ¹⁹F NMR (376 MHz, DMSO-d₆, ppm): δ −51.54 (3F), −91.07 (2F). |
| 320 | 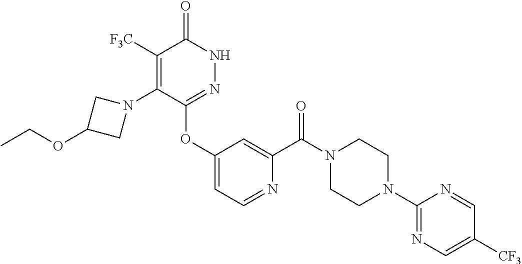 [SMILES: CCOC1CN(c2c(Oc3ccnc(C(=O)N4CCN(c5ncc(C(F)(F)F)cn5)CC4)c3)n[nH]c(=O)c2C(F)(F)F)C1] | 615.4 | 0.6TFA salt, ¹H NMR (400 MHz, DMSO-d₆, ppm): δ 11.90 (s, 1H), 8.75 (s, 2H), 8.61 (d, J = 5.6 Hz, 1H), 7.56 (s, 1H), 7.41 (d, J = 5.6 Hz, 1H), 4.66-4.52 (m, 2H), 4.30-4.14 (m, 3H), 4.04-3.93 (m, 2H), 3.92-3.82 (m, 2H), 3.81-3.67 (m, 2H), 3.63-3.51 (m, 2H), 3.48-3.37 (m, 2H), 1.12 (t, J = 7.0 Hz, 3H). ¹⁹F NMR (376 MHz, DMSO-d₆, ppm): δ −51.37 (3F), −59.30 (3F). |
| 321 | 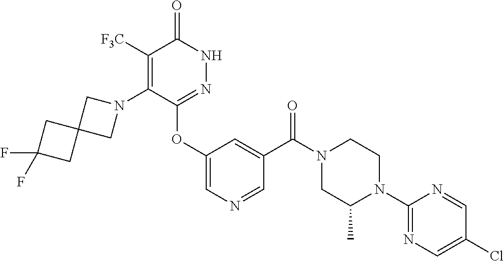 [SMILES: C[C@@H]1CN(C(=O)c2cncc(Oc3n[nH]c(=O)c(C(F)(F)F)c3N3CC4(C3)CC(F)(F)C4)c2)CCN1c1ncc(Cl)cn1] | 627.4 | 1.1TFA salt, ¹H NMR (400 MHz, methanol-d₄, ppm): δ 8.63 (s, 1H), 8.60-8.50 (m, 1H), 8.34 (s, 2H), 7.99-7.85 (m, 1H), 5.13-4.99 (m, 1H), 4.69 (s, 4H), 4.62-4.44 (m, 2H), 3.88- 3.71 (m, 1H), 3.63-3.51 (m, 1H), 3.43-3.33 (m, 1H), 3.29- 3.09 (m, 1H), 2.85 (t, J = 12.1 Hz, 4H), 1.33-1.06 (m, 3H). ¹⁹F NMR (376 MHz, methanol-d₄, ppm): δ −53.19 (3F), δ −94.70 (2F). |
| 322 | 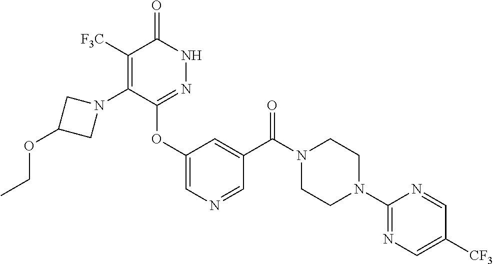 [SMILES: CCOC1CN(c2c(Oc3cncc(C(=O)N4CCN(c5ncc(C(F)(F)F)cn5)CC4)c3)n[nH]c(=O)c2C(F)(F)F)C1] | 615.4 | 1.2TFA salt, ¹H NMR (400 MHz, CDCl₃, ppm): δ 10.98 (brs, 1H), 8.62 (s, 1H), 8.58 (s, 1H), 8.53 (s, 2H), 7.75 (s, 1H), 4.84-4.72 (m, 2H), 4.61-4.48 (m, 2H), 4.40-4.30 (m, 1H), 4.15-3.79 (m, 6H), 3.77-3.57 (m, 2H), 3.54-3.43 (m, 2H), 1.27 (t, J = 7.0 Hz, 3H). ¹⁹F NMR (376 MHz, CDCl₃, ppm): δ −51.70 (3F), −61.16 (3F). |
| 323 | 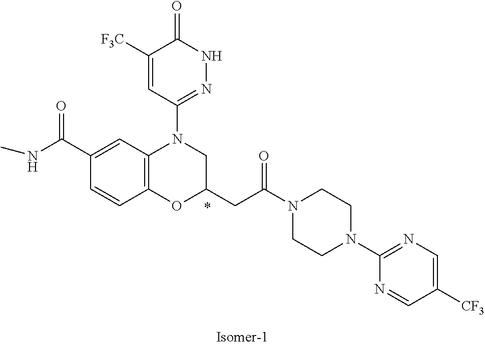 [SMILES: CNC(=O)c1ccc2c(c1)N(c1cc(C(F)(F)F)c(=O)[nH]n1)CC(CC(=O)N1CCN(c3ncc(C(F)(F)F)cn3)CC1)O2] | 627.2 | SFC analysis: 100% ee; retention time: 4.890 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.60 (s, 2H), 8.00 (s, 1H), 7.52 (d, J = 2.0 Hz, 1H), 7.42 (dd, J = 8.5, 2.1 Hz, 1H), 6.96 (d, J = 8.4 Hz, 1H), 4.76 (d, J = 4.8 Hz, 1H), 4.15 (dd, J = 13.3, 2.6 Hz, 1H), 4.02-3.90 (m, 4H), 3.78-3.64 (m, 4H), 3.56 (dd, J = 13.3, 7.5 Hz, 1H), 3.09-2.99 (m, 1H), 2.91-2.78 (m, 4H). |
| 324 | 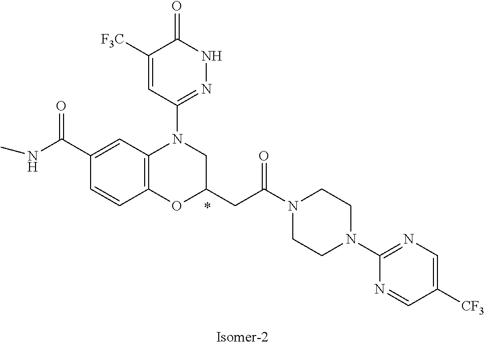 [SMILES: CNC(=O)c1ccc2c(c1)N(c1cc(C(F)(F)F)c(=O)[nH]n1)CC(CC(=O)N1CCN(c3ncc(C(F)(F)F)cn3)CC1)O2] | 627.2 | SFC analysis: 98.92% ee; retention time: 3.949 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.60 (s, 2H), 8.00 (s, 1H), 7.52 (d, J = 2.0 Hz, 1H), 7.42 (dd, J = 8.5, 2.1 Hz, 1H), 6.96 (d, J = 8.5 Hz, 1H), 4.81-4.69 (m, 1H), 4.15 (dd, J = 13.3, 2.6 Hz, 1H), 4.03-3.89 (m, 4H), 3.78- 3.62 (m, 4H), 3.56 (dd, J = 13.2, 7.5 Hz, 1H), 3.09-2.99 (m, 1H), 2.90-2.77 (m, 4H). |
| 325 | 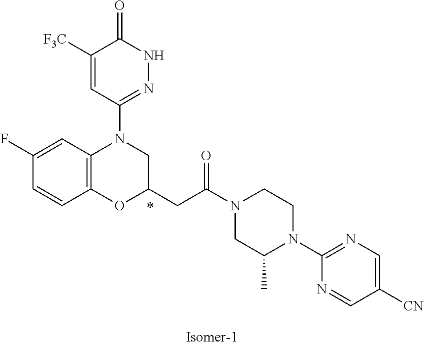 [SMILES: C[C@@H]1CN(C(=O)CC2CN(c3cc(C(F)(F)F)c(=O)[nH]n3)c3cc(F)ccc3O2)CCN1c1ncc(C#N)cn1] | 559.2 | SFC analysis: 100% ee; retention time: 1.403 min; column: DAICEL CHIRALPAK ® AD, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 8.00 (d, J = 10.4 Hz, 1H), 6.91-6.80 (m, 2H), 6.72-6.60 (m, 1H), 5.09-4.96 (m, 1H), 4.74-4.58 (m, 2H), 4.46-4.32 (m, 1H), 4.14-3.88 (m, 2H), 3.64-3.30 (m, 3H), 3.16-2.69 (m, 3H), 1.34-1.10 (m, 3H). |
| 326 | 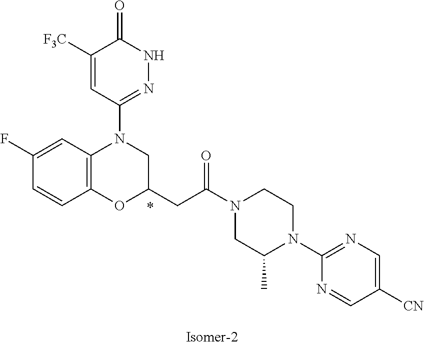 [SMILES: C[C@@H]1CN(C(=O)CC2CN(c3cc(C(F)(F)F)c(=O)[nH]n3)c3cc(F)ccc3O2)CCN1c1ncc(C#N)cn1] | 559.2 | SFC analysis: 100% ee; retention time: 1.723 min; column: DAICEL CHIRALPAK ® AD, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.64 (s, 2H), 7.99 (s, 1H), 6.90-6.78 (m, 2H), 6.71- 6.60 (m, 1H), 5.13-4.92 (m, 1H), 4.72-4.58 (m, 2H), 4.47- 4.35 (m, 1H), 4.16-3.83 (m, 2H), 3.62-3.32 (m, 3H), 3.16- 2.65 (m, 3H), 1.35-1.11 (m, 3H). |
| 327 | 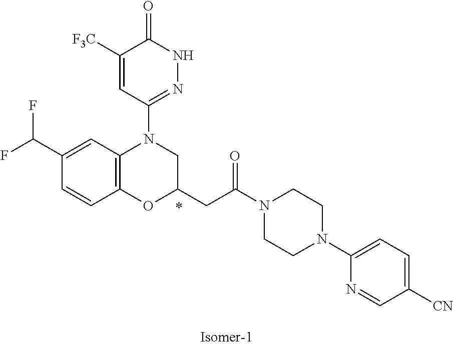 [SMILES: N#Cc1ccc(N2CCN(C(=O)CC3CN(c4cc(C(F)(F)F)c(=O)[nH]n4)c4cc(C(F)F)ccc4O3)CC2)nc1] | 576.2 | SFC analysis: 100% ee; retention time: 4.458 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol- d₄, ppm): δ 8.42 (s, 1H), 7.98 (s, 1H), 7.76 (dd, J = 9.0, 1.6 Hz, 1H), 7.23 (s, 1H), 7.11 (d, J = 8.5 Hz, 1H), 6.99 (d, J = 8.4 Hz, 1H), 6.87 (d, J = 9.1 Hz, 1H), 6.63 (t, J = 56.4 Hz, 1H), 4.79-4.68 (m, 1H), 4.16 (dd, J = 13.3, 2.1 Hz, 1H), 3.90-3.62 (m, 8H), 3.56 (dd, J = 13.2, 7.5 Hz, 1H), 3.08- 2.97 (m, 1H), 2.86-2.74 (m, 1H). |
| 328 | 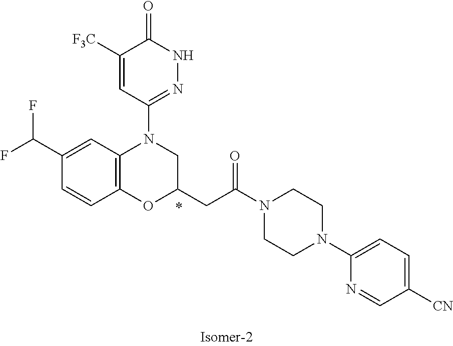 [SMILES: N#Cc1ccc(N2CCN(C(=O)CC3CN(c4cc(C(F)(F)F)c(=O)[nH]n4)c4cc(C(F)F)ccc4O3)CC2)nc1] | 576.2 | SFC analysis: 100% ee; retention time: 5.008 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. 0.4FA salt, ¹H NMR (400 MHz, methanol-d₄, ppm): δ 8.53 (s, 0.4H), 8.42 (d, J = 2.0 Hz, 1H), 7.99 (s, 1H), 7.76 (dd, J = 9.1, 2.3 Hz, 1H), 7.23 (s, 1H), 7.11 (d, J = 8.4 Hz, 1H), 6.99 (d, J = 8.4 Hz, 1H), 6.87 (d, J = 9.0 Hz, 1H), 6.63 (t, J = 56.4 Hz, 1H), 4.77- 4.69 (m, 1H), 4.16 (dd, J = 13.3, 2.6 Hz, 1H), 3.89-3.63 (m, 8H), 3.56 (dd, J = 13.3, 7.6 Hz, 1H), 3.08-2.97 (m, 1H), 2.87-2.74 (m, 1H). |
| 329 | 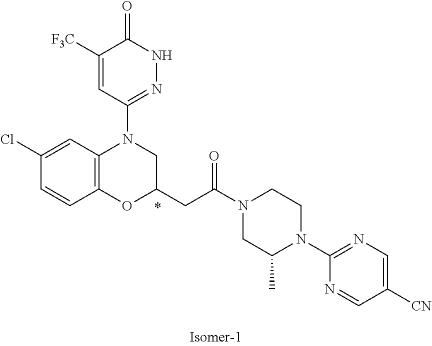 [SMILES: C[C@@H]1CN(C(=O)CC2CN(c3cc(C(F)(F)F)c(=O)[nH]n3)c3cc(Cl)ccc3O2)CCN1c1ncc(C#N)cn1] | 575.2 | SFC analysis: 100% ee; retention time: 2.654 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.99 (s, 1H), 7.08 (d, J = 2.1 Hz, 1H), 6.97-6.80 (m, 2H), 5.10-4.95 (m, 1H), 4.77-4.65 (m, 2H), 4.47-4.35 (m, 1H), 4.18-3.86 (m, 2H), 3.67-3.35 (m, 3H), 3.15-2.73 (m, 3H), 1.20 (d, J = 6.7 Hz, 3H). |
| 330 | 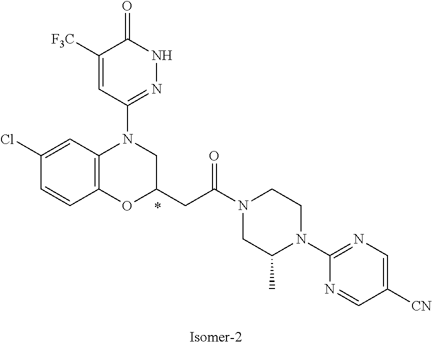 [SMILES: C[C@@H]1CN(C(=O)CC2CN(c3cc(C(F)(F)F)c(=O)[nH]n3)c3cc(Cl)ccc3O2)CCN1c1ncc(C#N)cn1] | 575.2 | SFC analysis: 100% ee; retention time: 3.533 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.63 (s, 2H), 7.99 (s, 1H), 7.09 (d, J = 2.2 Hz, 1H), 6.95-6.78 (m, 2H), 5.11-4.96 (m, 1H), 4.75-4.65 (m, 2H), 4.48-4.36 (m, 1H), 4.21-3.81 (m, 2H), 3.68-3.40 (m, 3H), 3.15-2.73 (m, 3H), 1.21 (d, J = 6.7 Hz, 3H). |
| 331 | 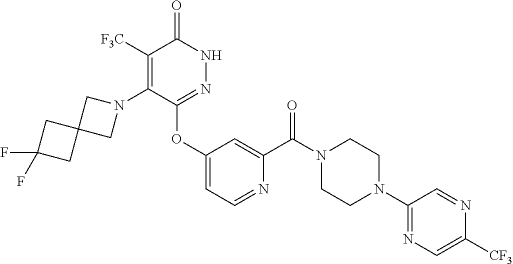 [SMILES: O=C(c1cc(Oc2n[nH]c(=O)c(C(F)(F)F)c2N2CC3(C2)CC(F)(F)C3)ccn1)N1CCN(c2cnc(C(F)(F)F)cn2)CC1] | 647.4 | ¹H NMR (400 MHz, DMSO-d₆, ppm): δ 11.89 (s, 1H), 8.63 (d, J = 5.6 Hz, 1H), 8.52 (s, 1H), 8.45 (s, 1H), 7.56 (s, 1H), 7.43 (d, J = 5.6 Hz, 1H), 4.51 (s, 4H), 3.95-3.70 (m, 6H), 3.69-3.58 (m, 2H), 2.95-2.79 (m, 4H). ¹⁹F NMR (376 MHz, DMSO-d₆, ppm): δ −51.55 (3F), −64.67 (3F), −91.05 (2F). |
| 332 | 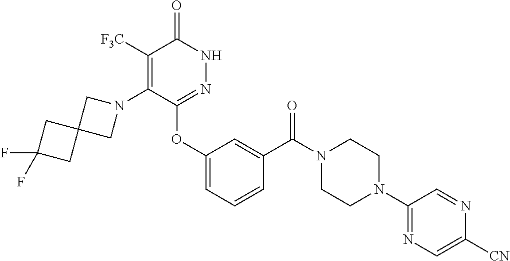 [SMILES: N#Cc1cnc(N2CCN(C(=O)c3cccc(Oc4n[nH]c(=O)c(C(F)(F)F)c4N4CC5(C4)CC(F)(F)C5)c3)CC2)cn1] | 603.4 | 0.4TFA salt, ¹H NMR (400 MHz, DMSO-d₆, ppm): δ 11.63 (s, 1H), 8.81 (s, 2H), 7.60-7.45 (m, 1H), 7.44-7.20 (m, 3H), 4.57 (s, 4H), 4.08-3.55 (m, 8H), 3.00-2.77 (m, 4H). ¹⁹F NMR (376 MHz, DMSO-d₆, ppm): δ −51.42 (3F), −91.09 (2F). |
| 333 | 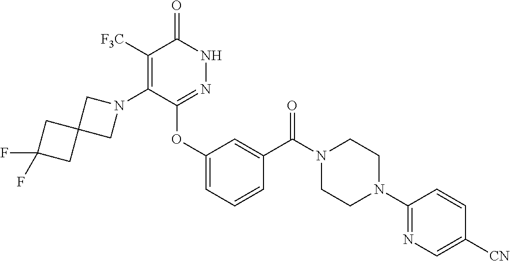 [SMILES: N#Cc1ccc(N2CCN(C(=O)c3cccc(Oc4n[nH]c(=O)c(C(F)(F)F)c4N4CC5(C4)CC(F)(F)C5)c3)CC2)nc1] | 602.4 | 1.9TFA salt, ¹H NMR (400 MHz, DMSO-d₆, ppm): δ 11.62 (s, 1H), 8.52 (s, 1H), 8.01-7.79 (m, 1H), 7.60-7.44 (m, 1H), 7.43-7.15 (m, 3H), 7.12-6.84 (m, 1H), 4.57 (s, 4H), 3.90- 3.50 (m, 8H), 2.98-2.72 (m, 4H). ¹⁹F NMR (376 MHz, DMSO-d₆, ppm): δ −51.42 (3F), −91.09 (2F). |
| 334 | 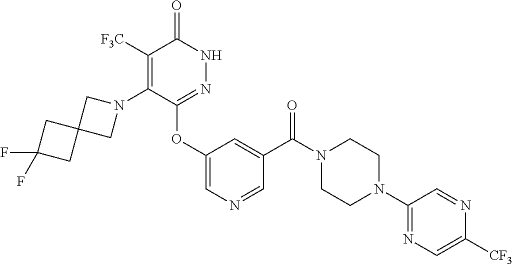 [SMILES: O=C(c1cncc(Oc2n[nH]c(=O)c(C(F)(F)F)c2N2CC3(C2)CC(F)(F)C3)c1)N1CCN(c2cnc(C(F)(F)F)cn2)CC1] | 647.4 | ¹H NMR (400 MHz, methanol-d₄, ppm): δ 8.63 (s, 1H), 8.57 (s, 1H), 8.42 (s, 1H), 8.32 (s, 1H), 7.92 (d, J = 2.4 Hz, 1H), 4.70 (s, 4H), 4.00-3.78 (m, 6H), 3.72-3.56 (m, 2H), 2.85 (t, J = 12.1 Hz, 4H). ¹⁹F NMR (376 MHz, methanol-d₄, ppm): δ −53.43 (3F), −67.92 (3F), −94.71 (2F). |
| 335 | 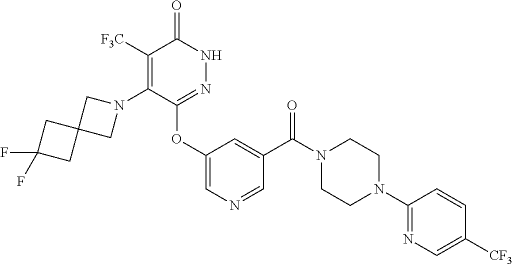 [SMILES: O=C(c1cncc(Oc2n[nH]c(=O)c(C(F)(F)F)c2N2CC3(C2)CC(F)(F)C3)c1)N1CCN(c2ccc(C(F)(F)F)cn2)CC1] | 646.4 | ¹H NMR (400 MHz, methanol- d₄, ppm) δ 8.62 (s, 1H), 8.56 (s, 1H), 8.37 (s, 1H), 7.91 (d, J = 2.6 Hz, 1H), 7.76 (dd, J = 9.1, 2.6 Hz, 1H), 6.93 (d, J = 9.0 Hz, 1H), 4.69 (s, 4H), 3.97- 3.69 (m, 6H), 3.67-3.57 (m, 2H), 2.85 (t, J = 12.1 Hz, 4H). ¹⁹F NMR (376 MHz, methanol-d₄, ppm): δ −53.43 (3F), −62.74 (3F), −94.70 (2F). |
| 336 | 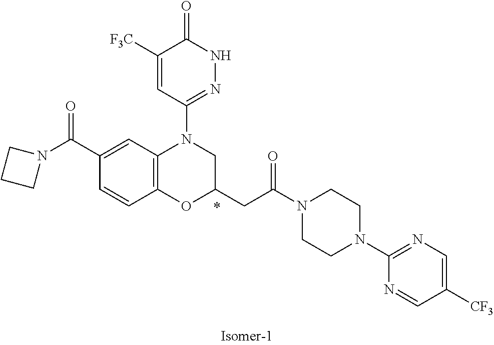 [SMILES: O=C(CC1CN(c2cc(C(F)(F)F)c(=O)[nH]n2)c2cc(C(=O)N3CCC3)ccc2O1)N1CCN(c2ncc(C(F)(F)F)cn2)CC1] | 653.2 | SFC analysis: 100% ee; retention time: 4.606 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.60 (s, 2H), 8.02 (s, 1H), 7.35 (d, J = 1.9 Hz, 1H), 7.26 (dd, J = 8.5, 1.9 Hz, 1H), 6.97 (d, J = 8.5 Hz, 1H), 4.80- 4.69 (m, 1H), 4.45-4.30 (m, 2H), 4.22-4.07 (m, 3H), 4.04- 3.87 (m, 4H), 3.80-3.62 (m, 4H), 3.55 (dd, J = 13.4, 7.6 Hz, 1H), 3.10-2.98 (m, 1H), 2.88-2.76 (m, 1H), 2.42-2.26 (m, 2H). |
| 337 | 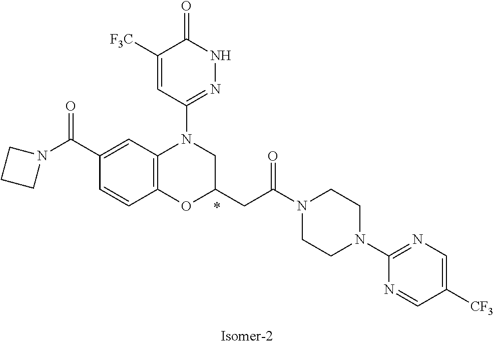 [SMILES: O=C(CC1CN(c2cc(C(F)(F)F)c(=O)[nH]n2)c2cc(C(=O)N3CCC3)ccc2O1)N1CCN(c2ncc(C(F)(F)F)cn2)CC1] | 653.2 | SFC analysis: 100% ee; retention time: 8.689 min; column: DAICEL CHIPAKCEL ® OZ, 100 × 3.0 mm, 3 μm, A for CO₂ and B for MeOH (0.1% DEA), 40%; pressure: 100 bar; flow rate: 1.5 mL/min. ¹H NMR (400 MHz, methanol-d₄, ppm): δ 8.60 (s, 2H), 8.02 (s, 1H), 7.35 (d, J = 1.9 Hz, 1H), 7.26 (dd, J = 8.5, 1.9 Hz, 1H), 6.97 (d, J = 8.4 Hz, 1H), 4.81- 4.67 (m, 1H), 4.45-4.30 (m, 2H), 4.21-4.10 (m, 3H), 4.03- 3.89 (m, 4H), 3.78-3.62 (m, 4H), 3.55 (dd, J = 13.2, 7.6 Hz, 1H), 3.10-2.98 (m, 1H), 2.88-2.77 (m, 1H), 2.41-2.25 (m, 2H). |
| 338 | 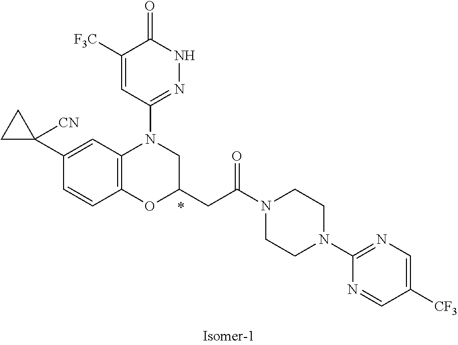 [SMILES: N#CC1(c2ccc3c(c2)N(c2cc(C(F)(F)F)c(=O)[nH]n2)CC(CC(=O)N2CCN(c4ncc(C(F)(F)F)cn4)CC2)O3)CC1] | 635.2 | SFC analysis: 95.36% ee; retention time: 3.900 min; column: DAICEL CHIRALPAK ® ID, 100 × 3 mm, 3 μm, A for CO₂ and B for MeOH (0.1% DEA), 5% to 40%; pressure: 100 bar; flow rate: 1.5 mL/min. ¹H NMR (400 MHz, methnaol-d₄, ppm): δ 8.59 (s, 2H), 8.01 (s, 1H), 7.05 (d, J = 1.6 Hz, 1H), 6.96-6.85 (m, 2H), 4.76-4.64 (m, 1H), 4.15 (dd, J = 13.2, 2.6 Hz, 1H), 4.01-3.87 (m, 4H), 3.75- 3.60 (m, 4H), 3.54 (dd, J = 13.2, 7.4 Hz, 1H), 3.06-2.96 (m, 1H), 2.84-2.74 (m, 1H), 1.66-1.53 (m, 2H), 1.42-1.24 (m, 2H). |
| 339 | 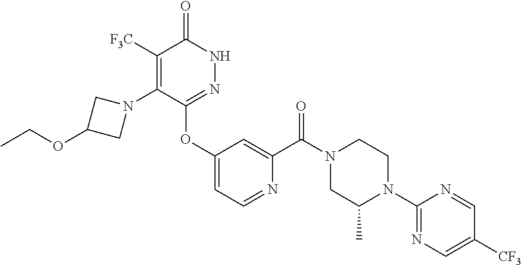 [SMILES: CCOC1CN(c2c(Oc3ccnc(C(=O)N4CCN(c5ncc(C(F)(F)F)cn5)[C@H](C)C4)c3)n[nH]c(=O)c2C(F)(F)F)C1] | 629.3 | ¹H NMR (400 MHz, CDCl₃, ppm): δ 11.15-10.95 (m, 1H), 8.59 (s, 1H), 8.52 (s, 1H), 7.52 (d, J = 7.2 Hz, 1H), 7.22- 7.13 (m, 1H), 5.19-4.96 (m, 1H), 4.78-4.56 (m, 4H), 4.46- 4.35 (m, 2H), 4.34-4.04 (m, 2H), 3.65-3.25 (m, 4H), 3.24- 3.06 (m, 1H), 1.33 (d, J = 6.8 Hz, 3H), 1.30-1.15 (m, 3H). ¹⁹F NMR (376 MHz, DMSO-d₆, ppm): δ −51.89 (3F), −61.06 (3F). |
| 340 | 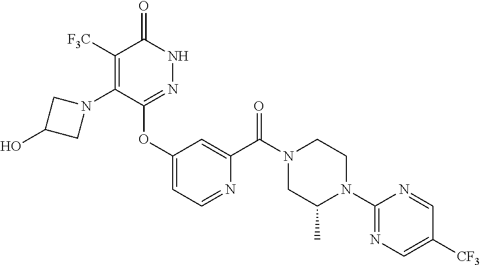 [SMILES: C[C@@H]1CN(C(=O)c2cc(Oc3n[nH]c(=O)c(C(F)(F)F)c3N3CC(O)C3)ccn2)CCN1c1ncc(C(F)(F)F)cn1] | 601.2 | 0.9TFA salt, ¹H NMR (400 MHz, CDCl₃, ppm): δ 10.93 (brs, 1H), 8.68-8.59 (m, 1H), 8.53 (s, 2H), 7.60-7.50 (m, 1H), 7.25-7.15 (m, 1H), 5.23-4.93 (m, 1H), 4.85-4.55 (m, 5H), 4.52-4.40 (m, 2H), 4.36-4.05 (m, 1H), 3.65-3.10 (m, 3H), 1.33 (d, J = 6.8 Hz, 3H); ¹⁹F NMR (376 MHz, CDCl₃, ppm): δ −51.84 (3F), −61.09 (3F). |
| 341 | 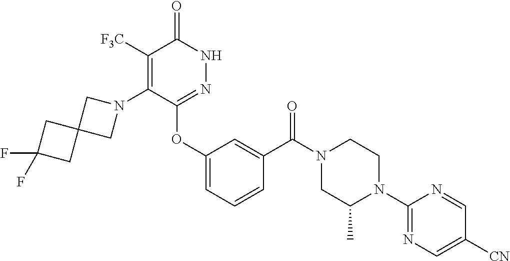 [SMILES: C[C@@H]1CN(C(=O)c2cccc(Oc3n[nH]c(=O)c(C(F)(F)F)c3N3CC4(C3)CC(F)(F)C4)c2)CCN1c1ncc(C#N)cn1] | 617.4 | ¹H NMR (400 MHz, DMSO-d₆, ppm): δ 11.65 (s, 1H), 8.83 (s, 2H), 7.60-7.50 (m, 1H), 7.48-7.21 (m, 3H), 5.15-4.80 (m, 1H), 4.69-4.32 (m, 6H), 3.77-3.49 (m, 2H), 3.25-2.99 (m, 2H), 2.92 (t, J = 12.5 Hz, 4H), 1.30- 1.16 (m, 3H). ¹⁹F NMR (376 MHz, DMSO-d₆, ppm): δ −51.43 (3F), −91.10 (2F). |
| 342 | 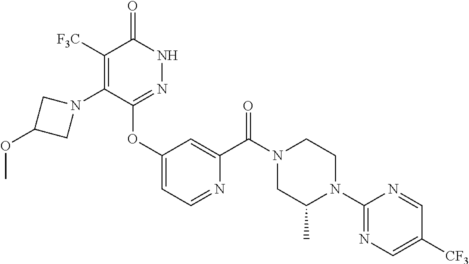 [SMILES: COC1CN(c2c(Oc3ccnc(C(=O)N4CCN(c5ncc(C(F)(F)F)cn5)[C@H](C)C4)c3)n[nH]c(=O)c2C(F)(F)F)C1] | 615.4 | 0.5TFA salt, ¹H NMR (400 MHz, DMSO-d₆, ppm): δ 11.99- 11.89 (m, 1H), 8.78 (s, 2H), 8.70-8.60 (m, 1H), 7.64-7.54 (m, 1H), 7.48-7.38 (m, 1H), 5.16-4.80 (m, 1H), 4.75-4.40 (m, 4H), 4.28-4.15 (m, 3H), 3.95-3.47 (m, 2H), 3.42-3.05 (m, 5H), 1.24-1.11 (m, 3H). ¹⁹F NMR (376 MHz, DMSO- d₆, ppm): δ −51.44 (3F), −59.30 (3F). |
| 343 | 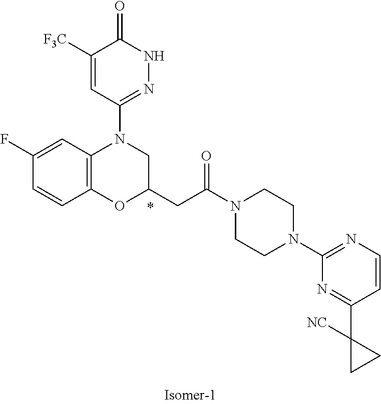 [SMILES: N#CC1(c2ccnc(N3CCN(C(=O)CC4CN(c5cc(C(F)(F)F)c(=O)[nH]n5)c5cc(F)ccc5O4)CC3)n2)CC1] | 585.2 | SFC analysis: 99.46% ee; retention time: 5.226 min; column: DAICEL CHIRALPAK ® AD, 100 × 3.0 mm, 3 μm, A for CO₂ and B for MeOH (0.1% DEA), 5% to 40%; pressure: 100 bar; flow rate: 1.5 mL/min. 0.2FA salt, ¹H NMR (400 MHz, methanol-d₄, ppm): δ 8.50 (s, 0.2 H), 8.31 (d, J = 5.1 Hz, 1H), 7.99 (s, 1H), 6.92-6.80 (m, 3H), 6.69- 6.61 (m, 1H), 4.70-4.54 (m, 1H), 4.09 (dd, J = 13.1, 2.5 Hz, 1H), 3.90-3.74 (m, 4H), 3.73-3.58 (m, 4H), 3.52 (dd, J = 13.1, 7.6 Hz, 1H), 3.05-2.94 (m, 1H), 2.84-2.72 (m, 1H), 1.85-1.72 (m, 4H). |
| 344 | 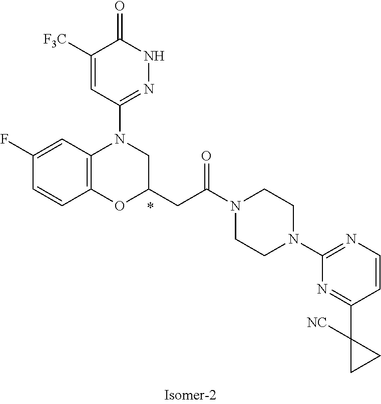 [SMILES: N#CC1(c2ccnc(N3CCN(C(=O)CC4CN(c5cc(C(F)(F)F)c(=O)[nH]n5)c5cc(F)ccc5O4)CC3)n2)CC1] | 585.2 | SFC analysis: 100% ee; retention time: 4.772 min; column: DAICEL CHIRALPAK ® AD, 100 × 3.0 mm, 3 μm, A for CO₂ and B for MeOH (0.1% DEA), 5% to 40%; pressures: 100 bar; flow rate: 1.5 mL/min. ¹H NMR (400 MHz, methanol-d₄, ppm): δ 8.31 (d, J = 5.1 Hz, 1H), 7.99 (s, 1H), 6.91-6.81 (m, 3H), 6.70-6.60 (m, 1H), 4.70-4.59 (m, 1H), 4.09 (dd, J = 13.1, 2.5 Hz, 1H), 3.90-3.74 (m, 4H), 3.73- 3.58 (m, 4H), 3.52 (dd, J = 13.1, 7.6 Hz, 1H), 3.04-2.94 (m, 1H), 2.83-2.72 (m, 1H), 1.84-1.72 (m, 4H). |
| 345 | 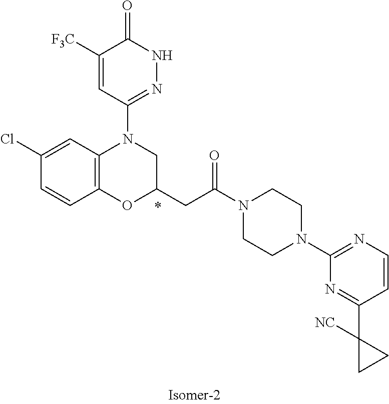 [SMILES: N#CC1(c2ccnc(N3CCN(C(=O)CC4CN(c5cc(C(F)(F)F)c(=O)[nH]n5)c5cc(Cl)ccc5O4)CC3)n2)CC1] | 601.2 | SFC analysis: 100% ee; retention time: 4.566 min; column: DAICEL CHIRALCEL ® AS, 100 × 3.0 mm, 3 μm, A for CO₂ and B for MeOH (0.1% DEA), 5% to 40%; pressure: 100 bar; flow rate: 1.5 mL/min. ¹H NMR (400 MHz, methanol-d₄, ppm): δ 8.31 (d, J = 5.1 Hz, 1H), 7.99 (s, 1H), 7.08 (d, J = 2.2 Hz, 1H), 6.96-6.78 (m, 3H), 4.71-4.59 (m, 1H), 4.10 (dd, J = 13.2, 2.6 Hz, 1H), 3.87-3.75 (m, 4H), 3.71-3.58 (m, 4H), 3.52 (dd, J = 13.2, 7.6 Hz, 1H), 3.05- 2.94 (m, 1H), 2.82-2.72 (m, 1H), 1.84-1.72 (m, 4H). |
| 346 | 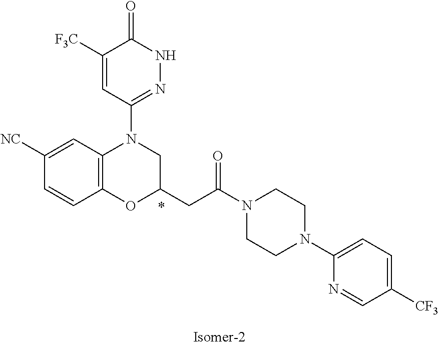 [SMILES: N#Cc1ccc2c(c1)N(c1cc(C(F)(F)F)c(=O)[nH]n1)CC(CC(=O)N1CCN(c3ccc(C(F)(F)F)cn3)CC1)O2] | 594.2 | ¹H NMR (400 MHz, methanol-d₄, ppm): δ 8.37 (s, 1H), 7.99 (s, 1H), 7.75 (dd, J = 9.1, 2.5 Hz, 1H), 7.52 (d, J = 1.8 Hz 1H), 7.27 (dd, J = 8.5, 1.8 Hz, 1H), 7.02 (d, J = 8.4 Hz, 1H), 6.90 (d, J = 9.0 Hz, 1H), 4.82- 4.72 (m, 1H), 4.15 (dd, J = 13.3, 2.5 Hz, 1H), 3.86-3.63 (m, 8H), 3.56 (dd, J = 13.3, 7.6 Hz, 1H), 3.09-2.99 (m, 1H), 2.89-2.78 (m, 1H). |
| 348 | 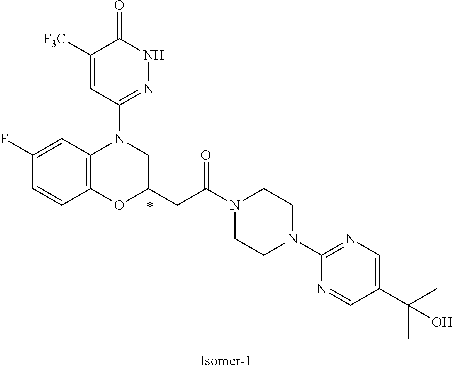 [SMILES: CC(C)(O)c1cnc(N2CCN(C(=O)CC3CN(c4cc(C(F)(F)F)c(=O)[nH]n4)c4cc(F)ccc4O3)CC2)nc1] | 560.2 [M − OH⁻]⁺ | SFC analysis: 100% ee; retention time: 5.066 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, DMSO-d₆, ppm): δ 13.11 (brs, 1H), 8.45 (s, 2H), 7.98 (s, 1H), 7.01 (dd, J = 10.6, 2.8 Hz, 1H), 6.86 (dd, J = 8.9, 5.6 Hz, 1H), 6.69 (td, J = 8.5, 2.9 Hz, 1H), 5.09 (s, 1H), 4.68-4.54 (m, 1H), 4.07-3.91 (m, 1H), 3.79-3.63 (m, 4H), 3.60-3.42 (m, 5H), 2.98-2.85 (m, 1H), 2.79-2.70 (m, 1H), 1.41 (s, 6H). |
| 349 | 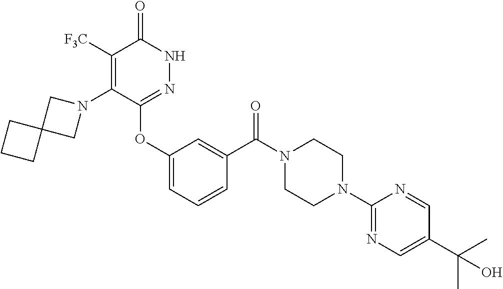 [SMILES: CC(C)(O)c1cnc(N2CCN(C(=O)c3cccc(Oc4n[nH]c(=O)c(C(F)(F)F)c4N4CC5(CCC5)C4)c3)CC2)nc1] | 582.4 [M − OH⁻]⁺ | 4.0TFA salt, ¹H NMR (400 MHz, DMSO-d₆, ppm): δ 11.55 (brs, 1H), 8.46 (s, 2H), 7.59-7.47 (m, 1H), 7.42-7.22 (m, 3H), 5.15 (s, 1H), 4.44 (s, 4H), 3.90- 3.58 (m, 8H), 2.25-2.10 (m, 4H), 1.80-1.68 (m, 2H), 1.41 (s, 6H). ¹⁹F NMR (376 MHz, DMSO-d₆, ppm): δ −51.24 (3F). |
| 350 | 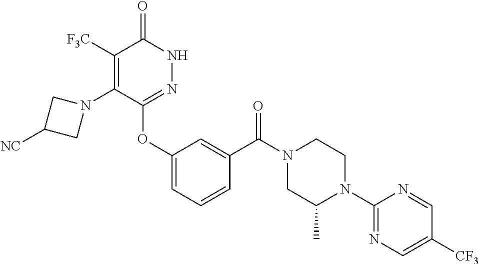 [SMILES: C[C@@H]1CN(C(=O)c2cccc(Oc3n[nH]c(=O)c(C(F)(F)F)c3N3CC(C#N)C3)c2)CCN1c1ncc(C(F)(F)F)cn1] | 609.4 | 0.5TFA salt, ¹H NMR (400 MHz, DMSO-d₆, ppm): δ 11.76 (s, 1H), 8.75 (s, 2H), 7.58-7.48 (m, 1H), 7.45-7.20 (m, 3H), 5.13-4.78 (m, 1H), 4.75-4.30 (m, 6H), 3.90-3.80 (m, 1H), 3.50-2.95 (m, 4H), 1.31-1.01 (m, 3H). ¹⁹F NMR (376 MHz, DMSO-d₆, ppm): δ −51.95 (3F), −59.30 (3F). |
| 351 | 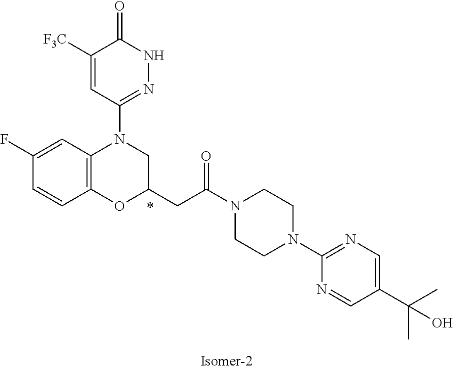 [SMILES: CC(C)(O)c1cnc(N2CCN(C(=O)CC3CN(c4cc(C(F)(F)F)c(=O)[nH]n4)c4cc(F)ccc4O3)CC2)nc1] | 560.2 [M − OH⁻]⁺ | SFC analysis: 100% ee; retention time: 5.175 min; column: DAICELCHIRALPAK ® OJ, 100 × 3.0 mm, 3 μm, A for CO₂ and B for MeOH (0.1% DEA), 5% to 40%; pressure: 100 bar; flow rate: 1.5 mL/min. ¹H NMR (400 MHz, DMSO-d₆, ppm): δ 13.15 (brs, 1H), 8.45 (s, 2H), 8.00 (s, 1H), 7.02 (dd, J = 10.7, 2.8 Hz, 1H), 6.86 (dd, J = 8.9, 5.5 Hz, 1H), 6.69 (td, J = 8.5, 2.8 Hz, 1H), 5.10 (s, 1H), 4.66-4.53 (m, 1H), 4.06-3.93 (m, 1H), 3.81-3.62 (m, 4H), 3.58-3.42 (m, 5H), 2.97-2.86 (m, 1H), 2.81-2.70 (m, 1H), 1.41 (s, 6H). |
| 352 | 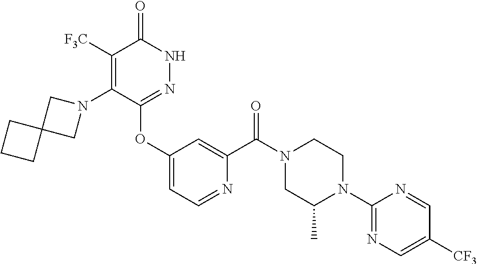 [SMILES: C[C@@H]1CN(C(=O)c2cc(Oc3n[nH]c(=O)c(C(F)(F)F)c3N3CC4(CCC4)C3)ccn2)CCN1c1ncc(C(F)(F)F)cn1] | 625.4 | 0.3TFA salt, ¹H NMR (400 MHz, DMSO-d₆, ppm): δ 11.90- 11.79 (m, 1H), 8.75 (s, 2H), 8.66- 8.56 (m, 1H), 7.58 (d, J = 15.2 Hz, 1H), 7.45-7.35 (m, 1H), 5.08-4.83 (m, 1H), 4.68- 4.35 (m, 6H), 3.95-3.83 (m, 1H), 3.58-3.41 (m, 1H), 3.40- 3.03 (m, 2H), 2.22-2.207 (m, 4H), 1.79-1.65 (m, 2H), 1.22- 1.08 (m, 3H). ¹⁹F NMR (376 MHz, DMSO-d₆, ppm): δ −51.37 (3F), −59.30 (3F). |
| 353 | 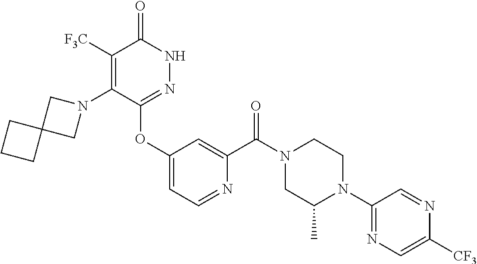 [SMILES: C[C@@H]1CN(C(=O)c2cc(Oc3n[nH]c(=O)c(C(F)(F)F)c3N3CC4(CCC4)C3)ccn2)CCN1c1cnc(C(F)(F)F)cn1] | 625.4 | 0.4TFA salt, ¹H NMR (400 MHz, DMSO-d₆, ppm): δ 11.89- 11.81 (m, 1H), 8.68-8.58 (m, 1H), 8.53 (s, 1H), 8.41 (d, J = 3.5 Hz, 1H), 7.59-7.51 (m, 1H), 7.46-7.36 (m, 1H), 4.87- 4.63 (m, 1H), 4.46-4.17 (m, 6H), 3.95-3.65 (m, 1H), 3.60- 3.50 (m, 1H), 3.40-3.12 (m, 2H), 2.20-2.05 (m, 4H), 1.78- 1.66 (m, 2H), 1.25-1.05 (m, 3H). ¹⁹F NMR (376 MHz, DMSO-d₆, ppm): δ −51.37 (3F), −64.67 (3F). |

### Biological Assay Example A: PARP7 TR-FRET Assay

A time-resolved fluorescence energy transfer (TR-FRET) assay was used to test binding activity of compounds to PARP7. Two microliter test compound was added in 384-well plate. Reactions were performed in a 6 uL volume by adding 4 uL mixture of PARP7 and Probe A (a biotinylated probe binding to the PARP7: N-(6-(2-(4-((2-(6-oxo-5-(trifluoromethyl)-1,6-dihydropyridazin-4-yl) isoindolin-5-yl) oxy) piperidin-1-yl) acetamido) hexyl)-5-((3aS,4S,6aR)-2-oxohexahydro-1H-thieno [3,4-d]imidazol-4-yl) pentanamide, made as reported in WO2019212937A1) in assay buffer (20 mM HEPES pH 8.0, 100 mM NaCl, 0.1% BSA, 2 mM DTT and 0.002% Tween20). The final concentration of PARP7 and Probe A were 6 nM and 2 nM, incubating with test compounds at 25° C. for 30 min. Four microliter ULight-anti 6xHis and LANCE Eu-W1024 labeled streptavidin (PerkinElmer) were added with the final concentration of ULight-anti 6xHis and LANCE Eu-W1024 at 4 nM and 0.25 nM. The reaction mixture was incubated at 25° C. for 30 min. The plate was read on a Tecan Spark plate reader (excitation wavelength at 320 nm, emission wavelength at 615 nm and 665 nm with a 90 us delay. The ratio of the 665/615 nm emission was calculated for each well to the amount of complex of PARP7 and Probe A in each well was calculated. The 0.25% DMSO vehicle was used as control and no PARP7 well was as blank well. Inhibition rate is calculated with the formula of % inhibition=100-100*(TRF_{cmpd}-TRF_{blank})/(TRF_{control}−TRF_{blank}). Inhibition IC50 is calculated with the equation of Y=Bottom+ (Top-Bottom)/(1+10\hat{ }((LogIC50−X) *Hill Slope))

Representative data are shown in Table 2 below.

TABLE 2
PARP7 TR-FRET of
Representative Compounds (IC₅₀)

| Com- | PARP7 |
|---|---|
| pound # | TR-FRET IC₅₀ |
| 1 | A |
| 2 | A |
| 3 | B |
| 4 | A |
| 5 | B |
| 6 | A |
| 7 | B |
| 8 | A |
| 9 | C |
| 10 | A |
| 11 | C |
| 12 | A |
| 13 | B |
| 14 | A |
| 15 | A |
| 16 | A |
| 17 | C |
| 18 | C |
| 19 | A |
| 20 | A |
| 21 | A |
| 22 | B |
| 23 | A |
| 24 | B |
| 25 | A |
| 26 | A |
| 27 | A |
| 28 | C |
| 29 | C |
| 30 | B |
| 31 | A |
| 32 | A |
| 33 | B |
| 34 | A |
| 35 | B |
| 36 | A |
| 37 | A |
| 38 | A |
| 39 | A |
| 40 | A |
| 41 | A |
| 42 | A |
| 43 | A |
| 44 | B |
| 45 | B |
| 46 | C |
| 47 | C |
| 48 | A |
| 49 | B |
| 50 | A |
| 51 | B |
| 52 | A |
| 53 | A |
| 54 | A |
| 55 | A |
| 56 | A |
| 57 | A |
| 58 | A |
| 59 | A |
| 60 | A |
| 61 | A |
| 62 | A |
| 63 | A |
| 64 | A |
| 65 | A |
| 66 | A |
| 67 | A |
| 68 | A |
| 69 | A |
| 70 | A |
| 71 | B |
| 72 | A |
| 73 | A |
| 74 | C |
| 75 | A |
| 76 | A |
| 77 | A |
| 78 | B |
| 79 | A |
| 80 | A |
| 81 | A |
| 82 | A |
| 83 | A |
| 84 | A |
| 85 | A |
| 86 | A |
| 87 | A |
| 88 | B |
| 89 | A |
| 90 | B |
| 91 | B |
| 92 | A |
| 93 | A |
| 94 | A |
| 95 | A |
| 96 | B |
| 97 | A |
| 98 | A |
| 99 | A |
| 100 | A |
| 101 | B |
| 102 | A |
| 103 | A |
| 104 | A |
| 105 | B |
| 106 | B |
| 107 | B |
| 108 | A |
| 109 | A |
| 110 | B |
| 111 | A |
| 112 | A |
| 113 | B |
| 114 | C |
| 115 | B |
| 116 | B |
| 117 | C |
| 118 | B |
| 119 | C |
| 120 | B |
| 121 | B |
| 122 | B |
| 123 | A |
| 124 | C |
| 125 | A |
| 126 | C |
| 127 | A |
| 128 | A |
| 129 | C |
| 130 | A |
| 131 | B |
| 132 | A |
| 133 | A |
| 134 | A |
| 135 | A |
| 136 | A |
| 137 | A |
| 138 | A |
| 139 | B |
| 140 | A |
| 141 | A |
| 142 | A |
| 143 | A |
| 144 | A |
| 145 | A |
| 146 | B |
| 147 | A |
| 148 | A |
| 149 | A |
| 150 | A |
| 151 | A |
| 152 | A |
| 153 | A |
| 154 | A |
| 155 | A |
| 156 | A |
| 157 | A |
| 158 | B |
| 159 | B |
| 160 | A |
| 161 | A |
| 162 | A |
| 163 | A |
| 164 | B |
| 165 | A |
| 166 | A |
| 167 | A |
| 168 | A |
| 169 | A |
| 170 | A |
| 171 | B |
| 172 | A |
| 173 | A |
| 174 | A |
| 175 | A |
| 176 | A |
| 177 | A |
| 178 | B |
| 179 | A |
| 180 | A |
| 181 | A |
| 182 | A |
| 183 | A |
| 184 | C |
| 185 | A |
| 186 | A |
| 187 | A |
| 188 | A |
| 189 | A |
| 190 | A |
| 191 | A |
| 192 | A |
| 193 | A |
| 194 | A |
| 195 | A |
| 196 | B |
| 197 | A |
| 198 | A |
| 199 | A |
| 200 | A |
| 201 | B |
| 202 | A |
| 203 | A |
| 204 | A |
| 205 | A |
| 206 | A |
| 207 | A |
| 208 | C |
| 209 | C |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | B |
| 223 | A |
| 224 | A |
| 225 | B |
| 226 | A |
| 227 | A |
| 228 | A |
| 229 | A |
| 230 | B |
| 231 | A |
| 232 | A |
| 233 | A |
| 234 | A |
| 235 | A |
| 236 | A |
| 237 | A |
| 238 | A |
| 239 | A |
| 240 | B |
| 241 | C |
| 242 | A |
| 243 | B |
| 244 | B |
| 245 | A |
| 246 | A |
| 247 | B |
| 248 | A |
| 249 | B |
| 250 | A |
| 251 | B |
| 252 | A |
| 253 | A |
| 254 | A |
| 255 | A |
| 256 | A |
| 257 | A |
| 258 | A |
| 259 | A |
| 260 | B |
| 261 | A |
| 262 | A |
| 263 | B |
| 264 | A |
| 265 | B |
| 266 | A |
| 267 | A |
| 268 | A |
| 269 | B |
| 270 | A |
| 271 | A |
| 272 | A |
| 273 | A |
| 274 | A |
| 275 | A |
| 276 | A |
| 277 | A |
| 278 | A |
| 279 | A |
| 280 | A |
| 281 | A |
| 282 | A |
| 283 | A |
| 284 | B |
| 285 | A |
| 286 | C |
| 287 | B |
| 288 | C |
| 289 | C |
| 290 | C |
| 291 | A |
| 292 | B |
| 293 | A |
| 294 | B |
| 295 | B |
| 296 | A |
| 297 | B |
| 298 | B |
| 299 | A |
| 300 | A |
| 301 | C |
| 302 | A |
| 303 | A |
| 304 | A |
| 305 | A |
| 306 | A |
| 307 | A |
| 308 | A |
| 309 | A |
| 310 | A |
| 311 | A |
| 312 | B |
| 313 | A |
| 314 | A |
| 315 | A |
| 316 | A |
| 317 | A |
| 318 | A |
| 319 | A |
| 320 | A |
| 321 | A |
| 322 | B |
| 323 | A |
| 324 | A |
| 325 | A |
| 326 | B |
| 327 | A |
| 328 | A |
| 329 | A |
| 330 | B |
| 331 | A |
| 332 | A |
| 333 | A |
| 334 | A |
| 335 | A |
| 336 | A |
| 337 | A |
| 338 | A |
| 339 | A |
| 340 | A |
| 341 | A |
| 342 | A |
| 343 | A |
| 344 | C |
| 345 | C |
| 346 | A |
| 347 | A |
| 348 | A |
| 349 | A |
| 350 | A |
| 351 | A |
| 352 | A |
| 353 | A |

(A: <100 nM;
B: 100 nM-1 uM (micromolar);
C: >1 uM)

### Biological Assay Example B: Inhibition of cancer cell growth in NCI-H1373 cells by PARP7 inhibitors

NCI-H1373 cells were cultured in RPMI 1640 medium supplemented with 10% fetal bovine serum, 100 U/mL penicillin and 100 ug/mL streptomycin in a 37° C., 5% CO₂ incubator. Fifteen hundred cells were seeded into each well of 96-well plate in RPMI1640 medium containing 10% fetal bovine serum and incubated overnight. Serial diluted compounds for each well were added at a final DMSO concentration of 0.5% and a day zero plate was collected for analysis. Compounds were incubated with the cells for 6 days. Cell growth was assessed using Cell-titer Glo reagent (Promega #G⁷⁵⁷²). The luminescence signal was collected on Tecan Spark plate reader. Cell growth is determined by correcting for the cell count on day zero. Inhibition rate is calculated with the formula of % inhibition=100*(DMSO control-compounds)/(DMSO control-Blank). Cell growth inhibition IC50 is calculated with the equation of Y-Bottom+ (Top-Bottom)/(1+10\hat{ }((LogIC50−X) *Hill Slope)).

Representative data are shown in Table 3 below.

TABLE 3
Inhibition of Cancer Cell Growth
of Representative Compounds
in H1373 cells (IC₅₀)

| Com- | H1373 Cell |
|---|---|
| pound # | Proliferation IC₅₀ |
| 1 | B |
| 2 | A |
| 8 | B |
| 14 | A |
| 15 | B |
| 16 | C |
| 21 | B |
| 26 | C |
| 27 | B |
| 32 | B |
| 34 | A |
| 35 | C |
| 39 | B |
| 40 | B |
| 42 | C |
| 43 | B |
| 48 | A |
| 50 | B |
| 53 | C |
| 54 | C |
| 57 | A |
| 63 | B |
| 67 | B |
| 71 | C |
| 73 | B |
| 78 | B |
| 86 | A |
| 92 | B |
| 101 | C |
| 114 | C |
| 123 | C |
| 138 | A |
| 140 | C |
| 145 | A |
| 149 | A |
| 156 | A |
| 168 | B |
| 179 | A |
| 188 | A |
| 199 | A |
| 216 | A |
| 223 | B |
| 229 | A |
| 236 | B |
| 252 | A |
| 256 | A |
| 266 | A |
| 272 | A |
| 280 | B |
| 291 | B |
| 300 | B |
| 309 | B |
| 316 | A |
| 321 | A |
| 324 | C |
| 331 | A |
| 336 | A |
| 337 | C |
| 339 | A |
| 346 | C |
| 349 | A |
| 353 | A |

(A: <100 nM;
B: 100 nM-1 uM (micromolar);
C: >1 uM)

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 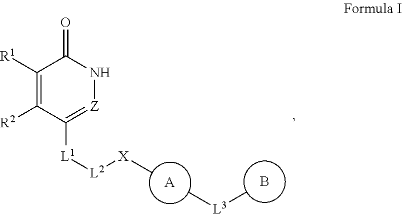
wherein:
Z is N or C, preferably N,
R¹ is hydrogen, halogen, CN, OR¹⁰, SR¹¹, S(O) R¹², S(O)₂R¹³, optionally substituted alkyl, optionally substituted alkenyl, optionally substituted alkynyl, optionally substituted carbocyclyl, optionally substituted heteroaryl, or optionally substituted heterocyclyl;
R² is hydrogen, halogen, CN, OR¹⁰, SR¹¹, S(O) R¹², S(O)₂R¹³, NR¹⁴R¹⁵, optionally substituted alkyl, optionally substituted alkenyl, optionally substituted alkynyl, optionally substituted carbocyclyl, optionally substituted phenyl, optionally substituted heteroaryl, or optionally substituted heterocyclyl;
L¹ and L² are independently null, O, S, S(O), S(O)₂, NR¹⁶, C(O), C(O) O, C(O) NR¹⁶, OC(O) NR¹⁶, S(O)₂NR¹⁶, NR¹⁷C(O) NR¹⁶, NR¹⁷S(O)₂NR¹⁶, optionally substituted alkylene, optionally substituted alkenylene, optionally substituted alkynylene, optionally substituted heteroalkylene, optionally substituted carbocyclylene, optionally substituted heterocyclylene, optionally substituted phenylene, or optionally substituted heteroarylene, preferably, L¹ and L² are not both null,
X is null, C(O), G¹-C(O)-G², S(O), S(O)₂, or G¹-S(O)₂-G², wherein G¹ and G² are each independently null, O, NH, optionally substituted C₁₋₄ alkylene, or optionally substituted C₁₋₄ heteroalkylene, or G¹ and G², together with the intervening atoms, are joined to form an optionally substituted 4-7 membered ring structure,
Ring A is an optionally substituted carbocyclic or heterocyclic ring,
L³ is null, O, S, S(O), S(O)₂, NR¹⁶, optionally substituted C₁₋₄ alkylene, or optionally substituted C₁₋₄ heteroalkylene,
Ring B is an optionally substituted aryl or heteroaryl ring,
or R¹ and R², together with the intervening atoms, are joined to form an optionally substituted cyclic structure;
or R² and L¹, together with the intervening atoms, are joined to form an optionally substituted cyclic structure;
or L¹ and L², together with the intervening atoms, are joined to form an optionally substituted cyclic structure;
or R¹, R², and L¹, together with the intervening atoms, are joined to form an optionally substituted cyclic structure;
or when L³ is null, ring A and ring B together represent an optionally substituted cyclic structure having one ring or at least two rings, e.g., a bicyclic structure;
wherein:
each of R¹⁰, R¹¹, R¹², and R¹³ at each occurrence is independently selected from hydrogen, optionally substituted alkyl, optionally substituted carbocyclyl, or optionally substituted heterocyclyl; and
each of R¹⁴, R¹⁵, R¹⁶, and R¹⁷ at each occurrence is independently selected from hydrogen, nitrogen protecting group, optionally substituted alkyl, optionally substituted cycloalkyl, or optionally substituted heterocyclyl.

2. The compound of claim 1, or a pharmaceutically acceptable salt thereof, wherein R¹ is halogen or CN.

3. The compound of claim 1, or a pharmaceutically acceptable salt thereof, wherein R¹ is C₁-4 alkyl, C₂₋₄ alkenyl, C₂₋₄ alkynyl, or C₃₋₆ cycloalkyl, each of which is optionally substituted with one or more (e.g., 1-3) substituents independently selected from F, OH, oxo, C₁₋₄ alkyl optionally substituted with 1-3 F, or C₁₋₄ alkoxy optionally substituted with 1-3 F.

4. The compound of claim 1, or a pharmaceutically acceptable salt thereof, wherein R¹ is OR¹⁰, SR¹¹, S(O) R¹², or S(O)₂R¹³, wherein R¹⁰, R¹¹, R¹², or R¹³ is independently hydrogen, C₁₋₄ alkyl, or C₃₋₆ cycloalkyl, wherein the C₁₋₄ alkyl or C₃₋₆ cycloalkyl is optionally substituted with one or more (e.g., 1-3) substituents independently selected from F, OH, oxo, C₁₋₄ alkyl optionally substituted with 1-3 F, and C₁₋₄ alkoxy optionally substituted with 1-3 F.

5. The compound of claim 1, or a pharmaceutically acceptable salt thereof, wherein R¹ is hydrogen, CH₃, ethyl, isopropyl, cyclopropyl, CN, OCH₃, SCH₃, CF₃, F, Cl, Br, CF₂H, 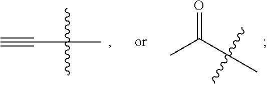
or R¹ is OCH₂CF₂H.

6. The compound of claim 1, or a pharmaceutically acceptable salt thereof, wherein R¹ is CF₃.

7. The compound of any one of claims 1-6, or a pharmaceutically acceptable salt thereof, wherein R² is C₁₋₄ alkyl, C₂₋₄ alkenyl, C₂₋₄ alkynyl, or C₃₋₆ cycloalkyl, each of which is optionally substituted with one or more (e.g., 1-5 or 1-3) substituents independently selected from (1) halo (preferably, F) or CN, (2) OH, (3) NG³G⁴, (4) oxo, (5) G⁵, and (6) OG⁵,
wherein:
G³ and G⁴ are independently hydrogen or G⁵, G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii) 4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)₅ or 6 membered heteroaryl having 1-3 ring heteroatoms, which is optionally substituted with 1-5 (e.g., 1, 2, or 3) G^{B}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and
G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

8. The compound of any one of claims 1-6, or a pharmaceutically acceptable salt thereof, wherein R² is NR¹⁴R¹⁵, wherein R¹⁴ and R¹⁵ are independently selected from (i) hydrogen, (ii) C₁₋₄ alkyl optionally substituted with 1-5 (e.g., 1, 2, or 3) G⁴, (iii) C₃₋₆ cycloalkyl optionally substituted with 1-5 (e.g., 1, 2, or 3) G^{A}, and (iv)₄-8 membered heterocyclyl having 1-3 ring heteroatoms, each independently selected from N, O, and S, which is optionally substituted with 1-5 (e.g., 1, 2, or 3) G^{A},
wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C};
wherein G^{C} at each occurrence is independently F, OH, C₁₋₄ alkyl optionally substituted with 1-3 F, or C₁₋₄ alkoxy optionally substituted with 1-3 F;
or R² is OR¹⁰, wherein R¹⁰ is (i) C₁₋₄ alkyl optionally substituted with 1-5 (e.g., 1, 2, or 3) G^{A3}, (ii) C₃₋₆ cycloalkyl optionally substituted with 1-5 (e.g., 1, 2, or 3) G^{A3}, or (iii)4-8 membered heterocyclyl having 1-3 ring heteroatoms, each independently selected from N, O, and S, which is optionally substituted with 1-5 (e.g., 1, 2, or 3) G^{A}3;
or R² is NHR¹⁵, wherein R¹⁵ is (i) C₁₋₄ alkyl optionally substituted with 1-5 (e.g., 1, 2, or 3) G^{A3}, (ii) C₃₋₆ cycloalkyl optionally substituted with 1-5 (e.g., 1, 2, or 3) G^{A3}, or (iii)4-8 membered heterocyclyl having 1-3 ring heteroatoms, each independently selected from N, O, and S, which is optionally substituted with 1-5 (e.g., 1, 2, or 3) G^{A}3;
wherein G⁴³ at each occurrence is independently halo (preferably, F) or CN; oxo; C₁₋₄ alkyl optionally substituted with 1-5 (e.g., 1, 2, or 3) G^{C3}; C₂₋₄ alkenyl optionally substituted with 1-5 (e.g., 1, 2, or 3) G^{C3}; C₂₋₄ alkynyl optionally substituted with 1-5 (e.g., 1, 2, or 3) G^{C3}; OH; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C3}; or a 3-8 membered ring optionally substituted with 1-5 (e.g., 1, 2, or 3) G^{C3};
wherein G^{C3} at each occurrence is independently is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F.

9. The compound of any one of claims 1-6, or a pharmaceutically acceptable salt thereof, wherein R² is 4-10 membered heterocyclyl having 1-3 ring heteroatoms, each independently selected from N, O, and S, which is optionally substituted with 1-5 (e.g., 1, 2, or 3) G⁴,
wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{c};
wherein G^{C} at each occurrence is independently F, OH, C₁₋₄ alkyl optionally substituted with 1-3 F, or C₁₋₄ alkoxy optionally substituted with 1-3 F;
R² is 4-10 membered heterocyclyl having 1-3 ring heteroatoms, each independently selected from N, O, and S, which is optionally substituted with 1-5 (e.g., 1, 2, or 3) G^{A3}, wherein G⁴³ at each occurrence is independently halo (preferably, F) or CN; oxo; C₁₋₄ alkyl optionally substituted with 1-5 (e.g., 1, 2, or 3) G^{C3}; C₂₋₄ alkenyl optionally substituted with 1-5 (e.g., 1, 2, or 3) G^{C3}; C₂₋₄ alkynyl optionally substituted with 1-5 (e.g., 1, 2, or 3) G^{C3}; OH; NH₂; NH (C₁₋₄ alkyl); N(C₁₋₄ alkyl) (C₁₋₄ alkyl); C₁₋₄ alkoxy optionally substituted with 1-5 (e.g., 1, 2, or 3) G^{C3}; or a 3-8 membered ring optionally substituted with 1-5 (e.g., 1, 2, or 3) G^{C3};
wherein G^{C3} at each occurrence is independently is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F.

10. The compound of claim 9, or a pharmaceutically acceptable salt thereof, wherein the 4-10 membered heterocyclyl is a 4-8 membered mono or bicyclic (fused, spiro, or bridged bicyclic) heterocyclyl having one or two ring heteroatoms, each independently selected from N, O, and S, such as 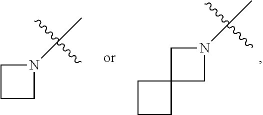 [SMILES: *N1CCC1 ; *N1CC2(CCC2)C1]
which is optionally substituted with 1-2 G^{A3}.

11. The compound of any one of claims 1-6, or a pharmaceutically acceptable salt thereof, wherein R² is hydrogen, CH₃, CF₃, 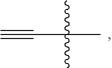 [SMILES: *C#C]
NH₂, NHCH₃, 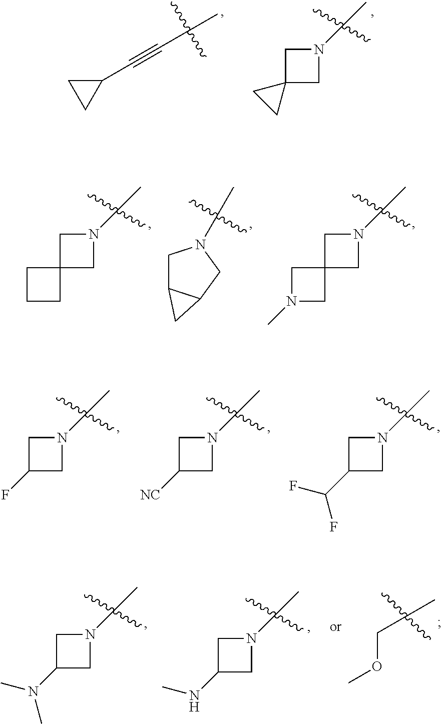
or R² is 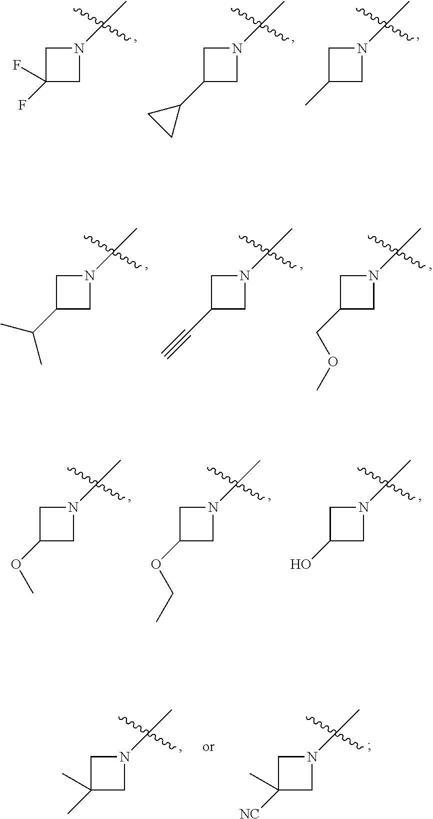
or R² is 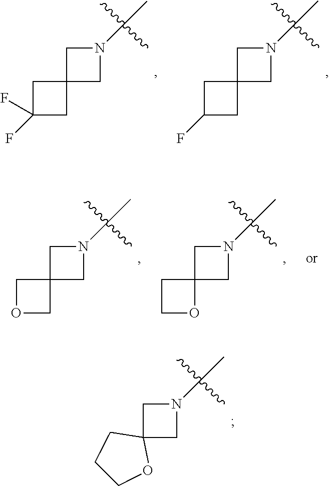 [SMILES: *N1CC2(C1)CC(F)(F)C2 ; *N1CC2(CC(F)C2)C1 ; *N1CC2(COC2)C1 ; *N1CC2(CCO2)C1 ; *N1CC2(CCCO2)C1]
or R² is 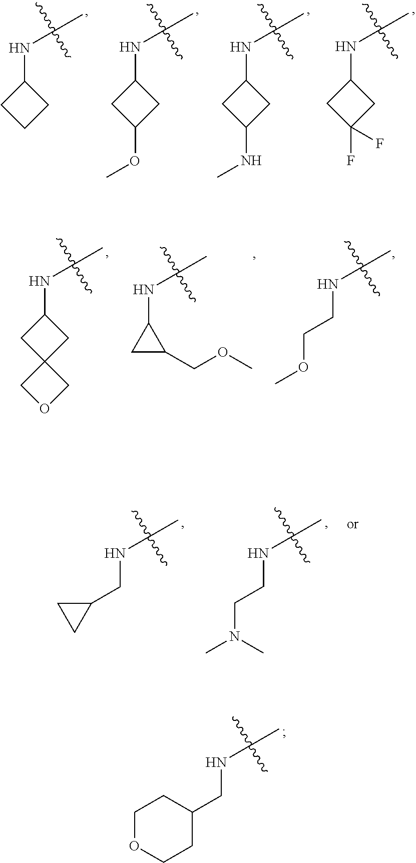
or R³ is 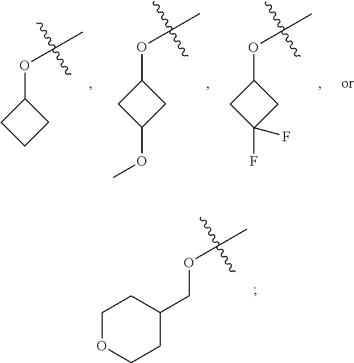
or R² is cyclopropyl.

12. The compound of claim 1, or a pharmaceutically acceptable salt thereof, wherein R¹ and R², together with the intervening atoms, are joined to form an optionally substituted phenyl ring or an optionally substituted 5 or 6 membered heteroaryl ring having 1-3 ring heteroatoms, each independently selected from N, O, and S.

13. The compound of claim 1, or a pharmaceutically acceptable salt thereof, wherein R¹ and R², together with the intervening atoms, are joined to form a phenyl ring, 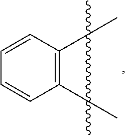
a pyridine ring, e.g., 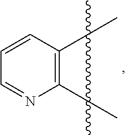
or a pyrrole ring, e.g., 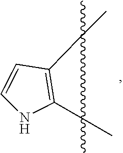
wherein each of the phenyl, pyridine, and pyrrole ring is optionally substituted with 1-5 (e.g., 1, 2, or 3) G^{B}, wherein G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F;
or each of the phenyl, pyridine, and pyrrole ring is optionally substituted with 1-5 (e.g., 1, 2, or 3) G^{B}3, wherein G^{B}3 at each occurrence is independently F, Cl, Br, CN, C₁₋₄ alkyl optionally substituted with 1-5 (e.g., 1, 2, or 3) G^{C3}, C₂₋₄ alkenyl optionally substituted with 1-5 (e.g., 1, 2, or 3) G^{C3}, C₂₋₄ alkynyl optionally substituted with 1-5 (e.g., 1, 2, or 3) G^{C3}, OH, C₃₋₆ cycloalkyl optionally substituted with 1-5 (e.g., 1, 2, or 3) G^{C}3, 4-6 membered heterocyclyl having 1-3 ring heteroatoms, each independently selected from N, O, and S, which is optionally substituted with 1-5 (e.g., 1, 2, or 3) G^{C}, NH₂, NH (C₁₋₄ alkyl), N(C₁₋₄ alkyl) (C₁₋₄ alkyl), C₁₋₄ alkoxy optionally substituted with 1-5 (e.g., 1, 2, or 3) G^{C3}, C₃₋₆ cycloalkoxy optionally substituted with 1-5 (e.g., 1, 2, or 3) G^{C}3, 4-6 membered heterocycloalkoxy optionally substituted with 1-5 (e.g., 1, 2, or 3) G^{C3}, or a 5 or 6-membered heteroaryl optionally substituted with 1-5 (e.g., 1, 2, or 3) G^{C3};
wherein G^{C3} at each occurrence is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F.

14. The compound of claim 1, or a pharmaceutically acceptable salt thereof, wherein R¹ and R², together with the intervening atoms, are joined to form a phenyl ring, 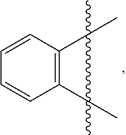
which is optionally substituted with one or more (e.g., 1-5 or 1-3) substituents independently selected from F, Cl, C₁₋₄ alkyl optionally substituted with 1-3 F, cyclopropyl, cyclobutyl,

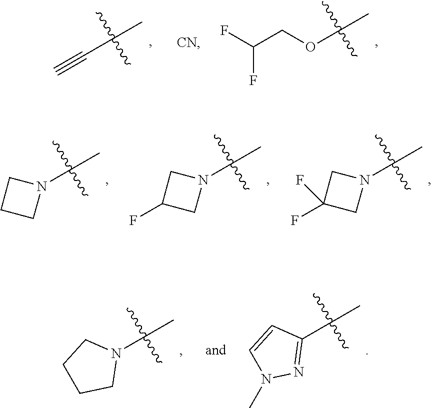

15. The compound of claim 1, or a pharmaceutically acceptable salt thereof, wherein R¹ and R², together with the intervening atoms, are joined to form a ring structure selected from: 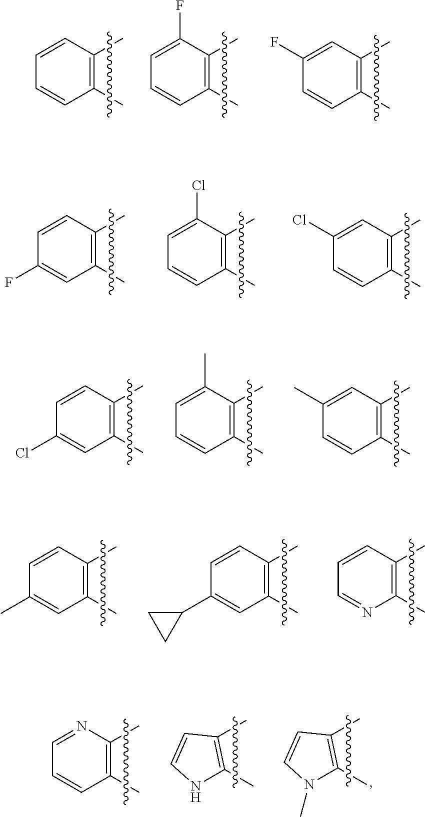
or selected from: 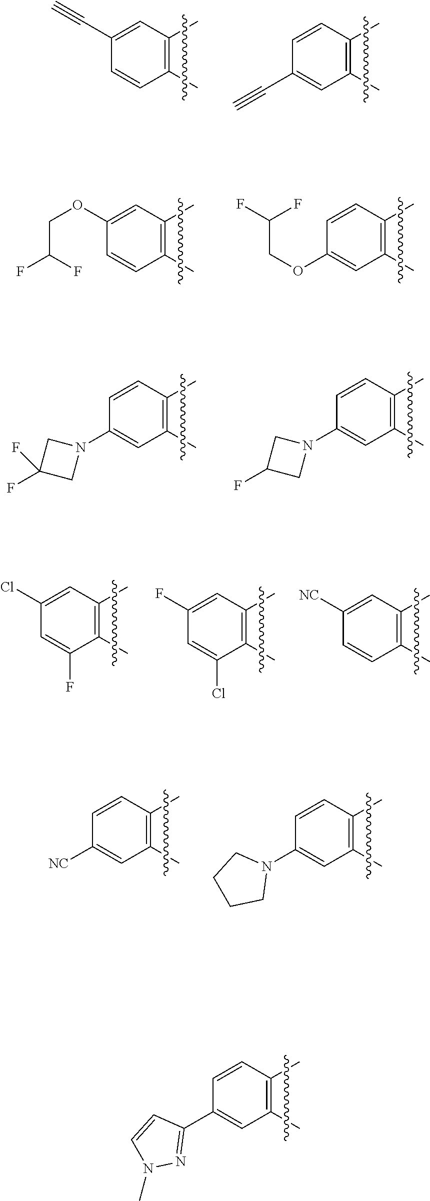
wherein the top connecting point of the fragments above is to the carbonyl group in Formula I.

16. The compound of claim 1 or a pharmaceutically acceptable salt thereof, wherein the compound is characterized as having Formula I-A-1, I-A-2, or I-A-3, 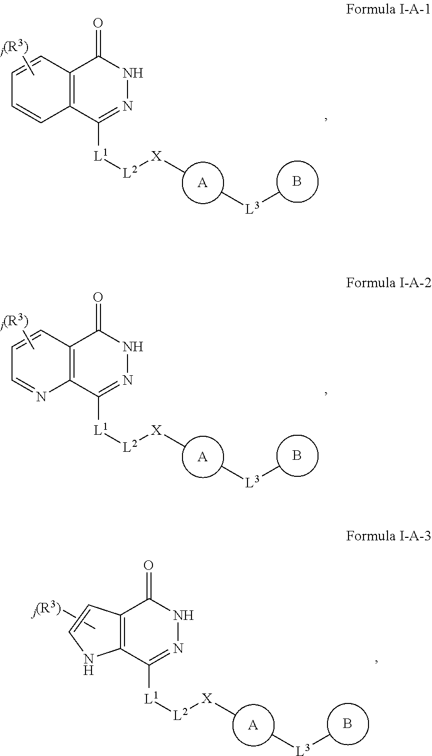
wherein:
j is 0, 1, 2, or 3, and
R³ at each occurrence is independently F, Cl, Br, CN, C₁₋₄ alkyl optionally substituted with 1-5 (e.g., 1, 2, or 3) G^{C}, OH, C₃₋₆ cycloalkyl optionally substituted with 1-5 (e.g., 1, 2, or 3) G^{C}, 4-6 membered heterocyclyl having 1-3 ring heteroatoms, each independently selected from N, O, and S, which is optionally substituted with 1-5 (e.g., 1, 2, or 3) G^{c}, NH₂, NH (C₁₋₄ alkyl), N(C₁₋₄ alkyl) (C₁₋₄ alkyl), C₁₋₄ alkoxy optionally substituted with 1-5 (e.g., 1, 2, or 3) G^{C}, C₃₋₆ cycloalkoxy optionally substituted with 1-5 (e.g., 1, 2, or 3) G^{c}, or 4-6 membered heterocycloalkoxy optionally substituted with 1-5 (e.g., 1, 2, or 3) G^{c}, wherein G^{C} at each occurrence is independently F, OH, C₁₋₄ alkyl optionally substituted with 1-3 F, or C₁₋₄ alkoxy optionally substituted with 1-3 F,
or R³ at each occurrence is independently F, Cl, Br, CN, C₁₋₄ alkyl optionally substituted with 1-5 (e.g., 1, 2, or 3) G^{C3}, C₂₋₄ alkenyl optionally substituted with 1-5 (e.g., 1, 2, or 3) G^{C3}, C₂₋₄ alkynyl optionally substituted with 1-5 (e.g., 1, 2, or 3) G^{C3}, OH, C₃₋₆ cycloalkyl optionally substituted with 1-5 (e.g., 1, 2, or 3) G^{C}3, 4-6 membered heterocyclyl having 1-3 ring heteroatoms, each independently selected from N, O, and S, which is optionally substituted with 1-5 (e.g., 1, 2, or 3) G^{C}, NH₂, NH (C₁₋₄ alkyl), N(C₁₋₄ alkyl) (C₁₋₄ alkyl), C₁₋₄ alkoxy optionally substituted with 1-5 (e.g., 1, 2, or 3) G^{C3}, C₃₋₆ cycloalkoxy optionally substituted with 1-5 (e.g., 1, 2, or 3) G^{C}3, 4-6 membered heterocycloalkoxy optionally substituted with 1-5 (e.g., 1, 2, or 3) G^{C3}, or a 5 or 6-membered heteroaryl optionally substituted with 1-5 (e.g., 1, 2, or 3) G^{C}3,
wherein G^{C3} at each occurrence is independently is independently (1) F, Cl, OH, or CN, (2) C₁₋₄ alkyl optionally substituted with 1-3 F, (3)3-4 membered ring (e.g., cyclopropyl, cyclobutyl, azetidinyl, oxetanyl, etc.) optionally substituted with 1-3 substituents independently F, OH, CN, or methyl, or (4) C₁₋₄ heteroalkyl having 1 or 2 heteroatoms independently O, N, or S, which is optionally substituted with 1-3 F,
or in Formula I-A-1 or I-A-3, one instance of R³ and L¹, together with the intervening atoms, are joined to form an optionally substituted 5-7 membered ring structure.

17. The compound of claim 16, or a pharmaceutically acceptable salt thereof, wherein j is 0.

18. The compound of claim 16, or a pharmaceutically acceptable salt thereof, wherein j is 1.

19. The compound of claim 18, or a pharmaceutically acceptable salt thereof, wherein R³ is F, Cl, C₁₋₄ alkyl optionally substituted with 1-3 F, cyclopropyl or cyclobutyl; or R³ is

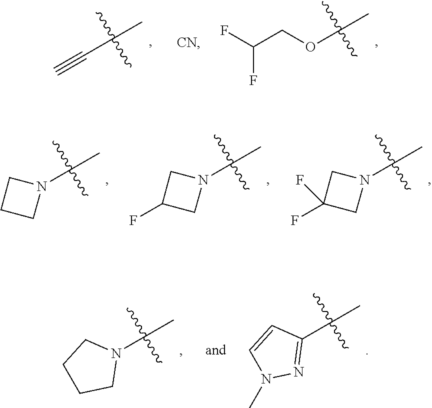

20. The compound of claim 16, or a pharmaceutically acceptable salt thereof, wherein the compound is characterized as having Formula I-A-1, wherein one instance of R³ and L¹, together with the intervening atoms, are joined to form an optionally substituted 5-7 membered ring structure such as a 5 or 6 membered ring structure containing 1 or 2 ring heteroatoms, each independently selected from N, O, and S.

21. The compound of claim 16, or a pharmaceutically acceptable salt thereof, wherein the compound is characterized as having Formula I-A-1-a, 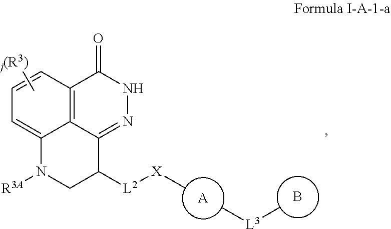
wherein:
j is 0, 1, or 2,
R³ is as defined in claim 16, and
R^{3A} is hydrogen, C₁₋₄ alkyl optionally substituted with 1-5 (e.g., 1, 2, or 3) G^{C}, C₃₋₆ cycloalkyl optionally substituted with 1-5 (e.g., 1, 2, or 3) G^{C}, or 4-6 membered heterocyclyl having 1-3 ring heteroatoms, each independently selected from N, O, and S, which is optionally substituted with 1-5 (e.g., 1, 2, or 3) G^{c},
wherein G^{C} at each occurrence is independently F, OH, C₁₋₄ alkyl optionally substituted with 1-3 F, or C₁₋₄ alkoxy optionally substituted with 1-3 F.

22. The compound of claim 21, or a pharmaceutically acceptable salt thereof, wherein j is 0.

23. The compound of claim 21, or a pharmaceutically acceptable salt thereof, wherein j is 1.

24. The compound of claim 23, or a pharmaceutically acceptable salt thereof, wherein R³ is F, Cl, C₁₋₄ alkyl optionally substituted with 1-3 F, cyclopropyl or cyclobutyl, or R³ is

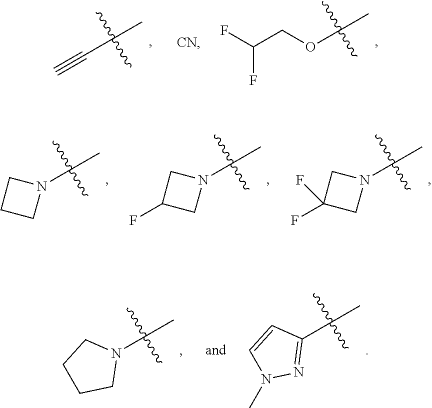

25. The compound of any one of claims 1-6, or a pharmaceutically acceptable salt thereof, wherein R² and L¹, together with the intervening atoms, are joined to form an optionally substituted 5-7 membered heterocyclyl having 1 or 2 heteroatoms independently selected from O, N, and S.

26. The compound of claim 25, or a pharmaceutically acceptable salt thereof, wherein when substituted, the 5-7 membered heterocyclyl is substituted with one or more (e.g., 1-5 or 1-3) substituents independently selected from (1) halo (preferably, F) or CN, (2) OH, (3) NG³G⁴, (4) oxo, (5) G⁵, (6) OG⁵, (7) (C₁₋₄ alkylene)-G⁵, and (8) (C₁₋₄ heteroalkylene)-G⁵, wherein:
G³ and G⁴ are independently hydrogen or G⁵, G⁵ at each occurrence is independently (i) C₁₋₄ alkyl optionally substituted with 1-5 (e.g., 1, 2, or 3) G⁴, (ii) C₃₋₆ cycloalkyl optionally substituted with 1-5 (e.g., 1, 2, or 3) G^{A}, (iii) 4-8 membered heterocyclyl having 1-3 ring heteroatoms, each independently selected from N, O, and S, which is optionally substituted with 1-5 (e.g., 1, 2, or 3) G⁴, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and
G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

27. The compound of claim 26, or a pharmaceutically acceptable salt thereof, wherein when substituted, the 5-7 membered heterocyclyl is substituted with one or more (e.g., 1-5 or 1-3) substituents independently selected from (1) F; (2) oxo; (3) G⁵; (4) (C₁₋₄ alkylene)-G⁵, and (6) (C₁₋₄ heteroalkylene)-G⁵, wherein G⁵ is as defined in claim 26.

28. The compound of claim 26, or a pharmaceutically acceptable salt thereof, wherein when substituted, the 5-7 membered heterocyclyl is substituted with one or more (e.g., 1-5 or 1-3) substituents independently selected from F, C₁₋₄ alkyl optionally substituted with 1-5 (e.g., 1, 2, or 3) GP and phenyl optionally substituted with 1-5 (e.g., 1, 2, or 3) G^{B}, wherein G″ at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F; wherein G^{B} is as defined in claim 26.

29. The compound of any one of claims 25-28, or a pharmaceutically acceptable salt thereof, wherein the 5-7 membered heterocyclyl has one ring heteroatom selected from N, S, and 0.

30. The compound of claim 25, or a pharmaceutically acceptable salt thereof, wherein the compound is characterized as having Formula I-B-1, I-B-2, or I-B-3: 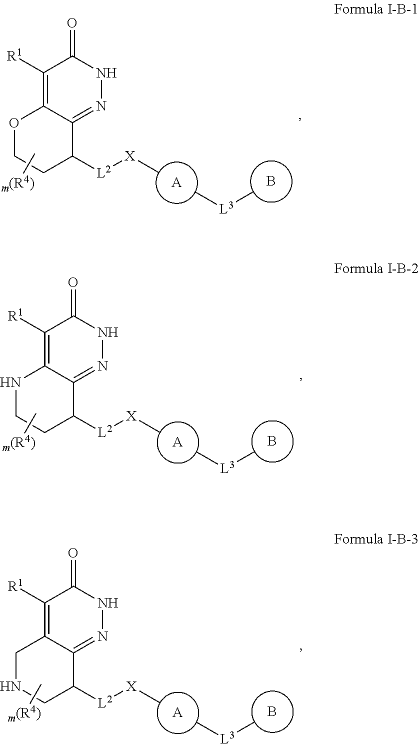
wherein:
m is 0, 1, 2, 3, or 4; and
R⁴ at each occurrence is independently (1) F, (2) OH, (3) NG³G⁴, (4) oxo, (5) G⁵, (6) OG⁵, (7) (C₁₋₄ alkylene)-G⁵, or (8) (C₁₋₄ heteroalkylene)-G⁵,
wherein:
G³ and G⁴ are independently hydrogen or G⁵, G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii) 4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and
G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F;
or in Formula I-B-2 or I-B-3, one instance of R⁴ and R¹, together with the intervening atoms, are joined to form an optionally substituted 5-7 membered ring structure;
or two instances of R⁴, together with the intervening atoms, are joined to form an optionally substituted 3-6 membered ring structure;
or one instance of R⁴ and L², together with the intervening atoms, are joined to form an optionally substituted 3-6 membered ring structure.

31. The compound of claim 30, or a pharmaceutically acceptable salt thereof, wherein m is 0.

32. The compound of claim 30, or a pharmaceutically acceptable salt thereof, wherein m is 1.

33. The compound of claim 32, or a pharmaceutically acceptable salt thereof, wherein R⁴ is C₁₋₄ alkyl optionally substituted with 1-5 (e.g., 1, 2, or 3) GP or phenyl optionally substituted with 1-5 (e.g., 1, 2, or 3) G^{B}, wherein GP at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F;
wherein G^{B} is as defined in claim 30.

34. The compound of claim 32, or a pharmaceutically acceptable salt thereof, wherein R⁴ is methyl, phenyl,

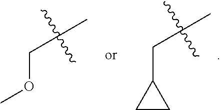

35. The compound of claim 30, or a pharmaceutically acceptable salt thereof, wherein the compound is characterized as having Formula I-B-3, and wherein one instance of R⁴ and R¹, together with the intervening atoms, are joined to form a ring structure selected from: 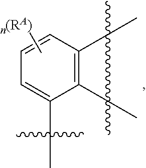
wherein RA is halogen, an optionally substituted C₁₋₄ alkyl, or optionally substituted C₃₋₆ cycloalkyl and n is 0, 1, or 2, wherein the top connecting point of the fragment is to the carbonyl group in Formula I-B-3.

36. The compound of claim 30, or a pharmaceutically acceptable salt thereof, wherein one instance of R⁴ and L², together with the intervening atoms, are joined to form an optionally substituted 3-6 membered ring structure, such as cyclopropyl.

37. The compound of any one of claims 1-19, or a pharmaceutically acceptable salt thereof, wherein L¹ is an optionally substituted 4-10 membered heterocyclylene having 1-3 ring heteroatoms, each independently selected from O, N, and S.

38. The compound of claim 37, or a pharmaceutically acceptable salt thereof, wherein when substituted, the 4-10 membered heterocyclylene is substituted with one or more (e.g., 1-5 or 1-3) substituents independently selected from (1) halo (preferably F or C₁) or CN, (2) OH, (3) NG³G⁴, (4) oxo, (5) G⁵, (6) OG⁵, (7) (C₁₋₄ alkylene)-G⁵, and (8) (C₁₋₄ heteroalkylene)-G⁵,
wherein:
G³ and G⁴ are independently hydrogen or G⁵, G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii) 4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B}, wherein G⁴ at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and
G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

39. The compound of claim 37, or a pharmaceutically acceptable salt thereof, wherein when substituted, the 4-10 membered heterocyclylene is substituted with one or more (e.g., 1-5 or 1-3) substituents, each independently F or C₁₋₄ alkyl optionally substituted with 1-5 (e.g., 1, 2, or 3) GP, wherein GD at each occurrence is independently F; OH; C₁₋₄ alkoxy optionally substituted with 1-3 F; C₃₋₆ cycloalkoxy optionally substituted with 1-3 F; or
C₃₋₆ cycloalkyl optionally substituted with 1-3 substituents independently selected from F, OH, and C₁₋₄ alkyl optionally substituted with 1-3 F.

40. The compound of claim 37-39, or a pharmaceutically acceptable salt thereof, wherein the 4-10 membered heterocyclylene is a 5 or 6 membered monocyclic heterocyclylene having one or two ring heteroatoms, each independently selected from N, O, and S.

41. The compound of any one of claims 37-39, or a pharmaceutically acceptable salt thereof, wherein the 4-10 membered heterocyclylene is a 6-10 membered fused, spiro, or bridged bicyclic heterocyclylene having one or two ring heteroatoms, each independently selected from N, O, and S.

42. The compound of any one of claims 37-39, or a pharmaceutically acceptable salt thereof, wherein the 4-10 membered heterocyclylene is selected from: 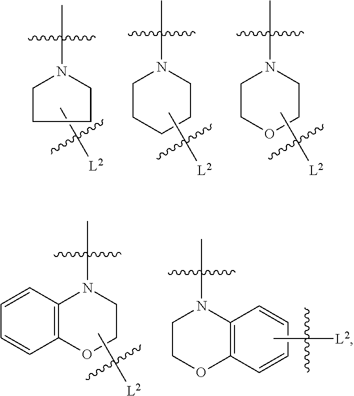
each of which is optionally substituted as defined in claims 37-39.

43. The compound of any one of claims 1-19, or a pharmaceutically acceptable salt thereof, wherein the compound is characterized as having Formula I-C-1, I-C-2, or I-C-3: 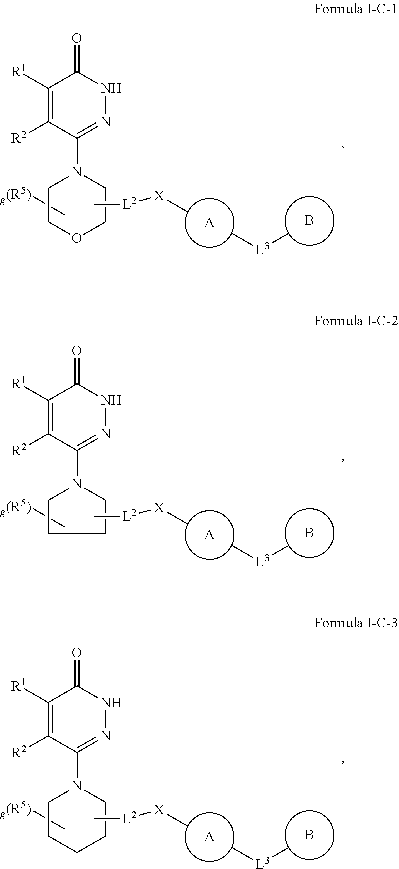
wherein:
g is 0, 1, 2, 3, or 4; and
R⁵ at each occurrence is independently selected from (1) halo (preferably F or C₁) or CN, (2) OH, (3) NG³G⁴, (4) oxo, (5) G⁵, (6) OG⁵, (7) (C₁₋₄ alkylene)-G⁵, and (8) (C₁₋₄ heteroalkylene)-G⁵,
wherein:
G³ and G⁴ are independently hydrogen or G⁵, G⁵ at each occurrence is independently (i) C₁₋₄ alkyl optionally substituted with 1-5 (e.g., 1, 2, or 3) G^{A}, (ii) C₃₋₆ cycloalkyl optionally substituted with 1-5 (e.g., 1, 2, or 3) G^{A}, (iii) 4-8 membered heterocyclyl having 1-3 ring heteroatoms, each independently selected from N, O, and S, which is optionally substituted with 1-5 (e.g., 1, 2, or 3) G^{A}, (iv) phenyl optionally substituted with 1-5 (e.g., 1, 2, or 3) G^{B}, or (v)5 or 6 membered heteroaryl having 1-3 ring heteroatoms, which is optionally substituted with 1-5 (e.g., 1, 2, or 3) G^{B}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C}; and
G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F;
or one instance of R⁵ and R², together with the intervening atoms, are joined to form an optionally substituted 5-7 membered ring structure;
or two instances of R⁵, together with the intervening atoms, are joined to form an optionally substituted 5-7 membered ring structure;
or one instance of R⁵ and L², together with the intervening atoms, are joined to form an optionally substituted 5-7 membered ring structure.

44. The compound of claim 43, or a pharmaceutically acceptable salt thereof, wherein R⁵ at each occurrence is independently F or C₁₋₄ alkyl optionally substituted with 1-5 (e.g., 1, 2, or 3) GP, wherein GP at each occurrence is independently F; OH; C₁₋₄ alkoxy optionally substituted with 1-3 F; C₃₋₆ cycloalkoxy optionally substituted with 1-3 F; or C₃₋₆ cycloalkyl optionally substituted with 1-3 substituents independently selected from F, OH, and C₁₋₄ alkyl optionally substituted with 1-3 F.

45. The compound of claim 43, or a pharmaceutically acceptable salt thereof, wherein g is 0.

46. The compound of claim 43, or a pharmaceutically acceptable salt thereof, wherein g is 1 or 2, and R⁵ at each occurrence is independently F or methyl.

47. The compound of claim 43, or a pharmaceutically acceptable salt thereof, wherein one instance of R⁵ and R², together with the intervening atoms, are joined to form an optionally substituted 5-7 membered ring structure.

48. The compound of claim 43, or a pharmaceutically acceptable salt thereof, wherein one instance of R⁵ and L², together with the intervening atoms, are joined to form an optionally substituted 5-7 membered ring structure, such as an optionally substituted phenyl.

49. The compound of claim 43, or a pharmaceutically acceptable salt thereof, wherein two instances of R⁵ together with the intervening atoms, are joined to form an optionally substituted 3-7 membered ring structure, such as an optionally substituted phenyl or optionally substituted pyridyl.

50. The compound of claim 43, or a pharmaceutically acceptable salt thereof, wherein the compound is characterized as having Formula I-C-1-a: 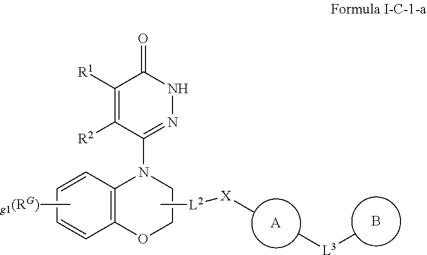
wherein:
R^{G} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{C};
wherein G^{C} at each occurrence is independently F; OH; C₁₋₄ alkyl optionally substituted with 1-3 F; or C₁₋₄ alkoxy optionally substituted with 1-3 F;
or R^{G} at each occurrence is independently halo (preferably, F, Cl, or Br); CN; OH; NH₂;
G^{A4}; OG^{A4}, NHG^{A4}, N(C₁₋₄ alkyl) (G^{A4}); COG^{A4}; SO₂G^{A4}; CONHG^{A4}; CON(C₁₋₄ alkyl) (G^{A4}); NHCOG^{A4}; or N(C₁₋₄ alkyl) COG^{A4};
wherein G^{A4} at each occurrence is independently (1) C₁₋₄ alkyl optionally substituted with 1-5 (e.g., 1, 2, or 3) G^{C4}; (2) C₂₋₄ alkenyl optionally substituted with 1-5 (e.g., 1, 2, or 3) G^{C4}; (3) C₂₋₄ alkynyl optionally substituted with 1-5 (e.g., 1, 2, or 3) G^{C4}; (4) C₁₋₄ heteroalkyl having 1 or 2 heteroatoms independently N, O, or S, wherein the S, if present, is optionally oxidized in the form of SO or SO₂, wherein the C₁₋₄ heteroalkyl is optionally substituted with 1-5 (e.g., 1, 2, or 3) G^{C4}; (6) C₃₋₆ cycloalkyl optionally substituted with 1-5 (e.g., 1, 2, or 3) G^{C4}; (7)4-6 membered heterocyclyl having 1-3 ring heteroatoms, each independently selected from N, O, and S, which is optionally substituted with 1-5 (e.g., 1, 2, or 3) G^{C4}; or (8) phenyl or 5 or 6-membered heteroaryl, each of which is optionally substituted with 1-5 (e.g., 1, 2, or 3) G^{C}4;
wherein G^{C4} at each occurrence is independently (a) halogen (e.g., F, Cl), OH, oxo (as applicable), or CN, (b) C₁₋₄ alkyl optionally substituted with 1-3 F, (c)3-4 membered ring (e.g., cyclopropyl, cyclobutyl, azetidinyl, oxetanyl, etc.) optionally substituted with 1-3 substituents independently F, OH, CN, or methyl, or (d) C₁₋₄ heteroalkyl having 1 or 2 heteroatoms independently O, N, or S, wherein the S, if present, is optionally oxidized in the form of SO or SO₂, wherein the C₁₋₄ heteroalkyl is which is optionally substituted with 1-3 F, and
g1 is an integer selected from 0, 1, 2, or 3, preferably, 0 or 1.

51. The compound of claim 50, or a pharmaceutically acceptable salt thereof, wherein g1 is 0.

52. The compound of claim 50, or a pharmaceutically acceptable salt thereof, wherein g1 is 1, preferably, R^{G} is para to the oxygen atom or para to the nitrogen atom, e.g., the compound of Formula I has a structure according to Formula I-C-1-al or I-C-1-a2:

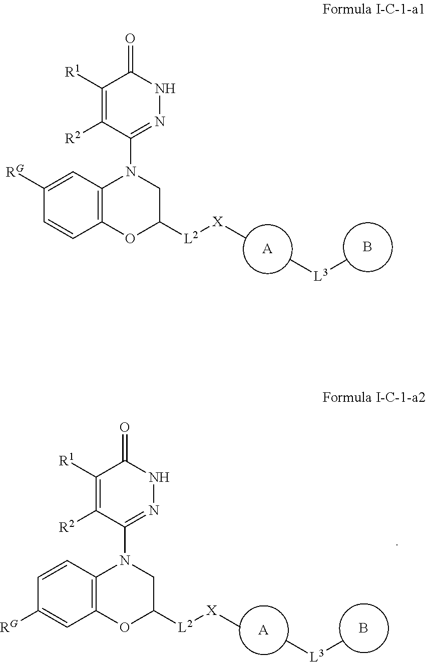

53. The compound of claim 50 or 52, or a pharmaceutically acceptable salt thereof, wherein R^{G} at each occurrence is independently F, Cl, CN, C₁₋₄ alkyl optionally substituted with 1-3 F (e.g., CHF₂), or C₃₋₆ cycloalkyl (preferably, cyclopropyl), or R^{G} at each occurrence is independently F, Cl, CN, C₁₋₄ alkyl optionally substituted with 1-3 F, cyclopropyl,

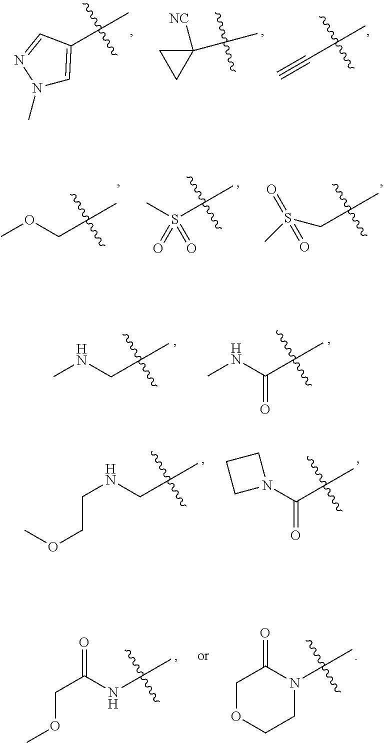

54. The compound of any one of claims 1-19, or a pharmaceutically acceptable salt thereof, wherein L¹ is O, S, or NR¹⁶, wherein R¹⁶ is hydrogen or an optionally substituted C₁₋₄ alkyl.

55. The compound of any one of claims 1-19, or a pharmaceutically acceptable salt thereof, wherein L¹ is O.

56. The compound of any one of claims 1-35, 37-47, and 49-55, or a pharmaceutically acceptable salt thereof, wherein L² is null, O, optionally substituted C₁₋₄ alkylene, optionally substituted C₁₋₄ heteroalkylene, optionally substituted C₃₋₆ cycloalkylene, optionally substituted 3-8 membered heterocyclylene having 1-3 ring heteroatoms, each independently selected from N, O, and S, optionally substituted phenylene, or optionally substituted 5 or 6 membered heteroarylene having 1-3 ring heteroatoms, each independently selected from N, O, and S.

57. The compound of any one of claims 1-35, 37-47, and 49-55, or a pharmaceutically acceptable salt thereof, wherein L² is null.

58. The compound of any one of claims 1-35, 37-47, and 49-55, or a pharmaceutically acceptable salt thereof, wherein L² is a C₁₋₄ alkylene.

59. The compound of any one of claims 1-35, 37-47, and 49-55, or a pharmaceutically acceptable salt thereof, wherein L² is a C₁₋₄ heteroalkylene having 1 or 2 heteroatoms independently selected from O, S, and N.

60. The compound of any one of claims 1-35, 37-47, and 49-55, or a pharmaceutically acceptable salt thereof, wherein L² is a C₁₋₄ heteroalkylene having 1 heteroatom which is O.

61. The compound of any one of claims 1-35, 37-47, and 49-55, or a pharmaceutically acceptable salt thereof, wherein L² is optionally substituted phenylene, wherein when substituted, the phenylene is substituted with 1-5 (e.g., 1, 2, or 3) G^{B},
wherein G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

62. The compound of any one of claims 1-35, 37-47, and 49-55, or a pharmaceutically acceptable salt thereof, wherein L² is optionally substituted 5 or 6-membered heteroarylene having 1-3 ring heteroatoms, each independently selected from N, O, and S, wherein when substituted, the heteroarylene is substituted with 1-5 (e.g., 1, 2, or 3) G^{B}, wherein G^{B} at each occurrence is independently halo (preferably, F, Cl, or Br); CN; C₁₋₄ alkyl optionally substituted with 1-5 (e.g., 1, 2, or 3) G^{C}; OH; C₃₋₆ cycloalkyl optionally substituted with 1-5 (e.g., 1, 2, or 3) G^{C}; 4-6 membered heterocyclyl having 1-3 ring heteroatoms, each independently selected from N, O, and S, which is optionally substituted with 1-5 (e.g., 1, 2, or 3) G^{C}; NH₂; NH (C₁₋₄ alkyl); N(C₁₋₄ alkyl) (C₁₋₄ alkyl);
C₁₋₄ alkoxy optionally substituted with 1-5 (e.g., 1, 2, or 3) G^{C}; C₃₋₆ cycloalkoxy optionally substituted with 1-5 (e.g., 1, 2, or 3) G^{C}; or 4-6 membered heterocycloalkoxy optionally substituted with 1-5 (e.g., 1, 2, or 3) G^{c};
wherein G^{C} at each occurrence is independently F; OH; C₁₋₄ alkyl optionally substituted with 1-3 F; or C₁₋₄ alkoxy optionally substituted with 1-3 F.

63. The compound of any one of claims 1-35, 37-47, and 49-55, or a pharmaceutically acceptable salt thereof, wherein L² is null, CH₂, CH₂CH₂, O,

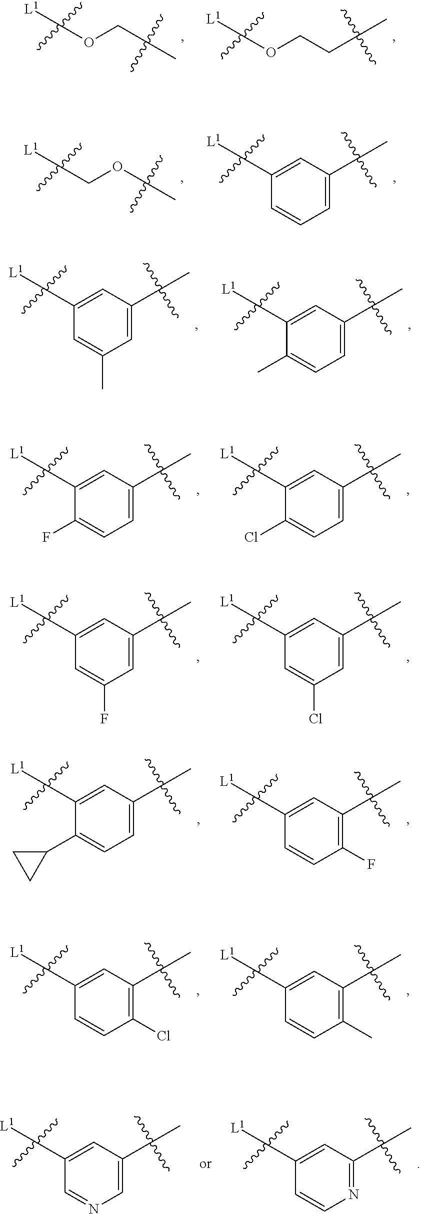

64. The compound of any one of claim 1-19, or a pharmaceutically acceptable salt thereof, wherein the compound is characterized as having a Formula I-D-1, I-D-2, or I-D-3: 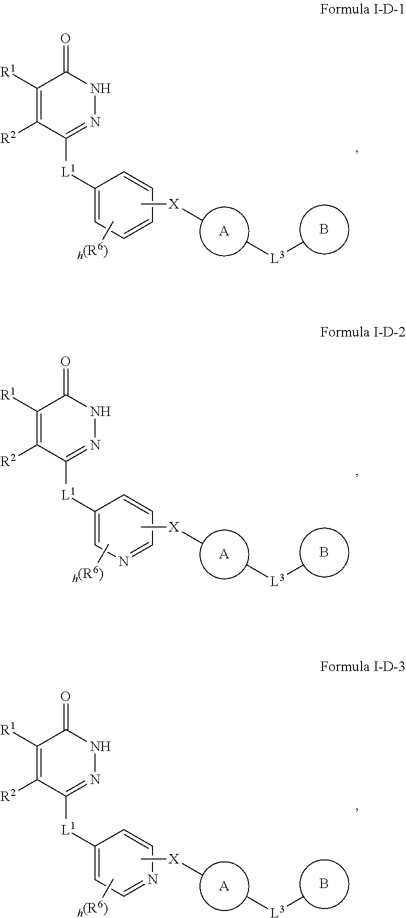
wherein:
L¹ is O, S, NH, or NCH₃,
h is 0, 1, or 2, and
R⁶ at each occurrence is independently F, Cl, Br, CN, C₁₋₄ alkyl optionally substituted with 1-5 (e.g., 1, 2, or 3) G^{C}, OH, cyclopropyl, cyclobutyl, 4-6 membered heterocyclyl having 1-3 ring heteroatoms, each independently selected from N, O, and S, which is optionally substituted with 1-5 (e.g., 1, 2, or 3) G^{C}, NH₂, NH (C₁₋₄ alkyl), N(C₁₋₄ alkyl) (C₁₋₄ alkyl), or C₁₋₄ alkoxy optionally substituted with 1-5 (e.g., 1, 2, or 3) G^{c}, wherein G^{C} at each occurrence is independently F, OH, C₁₋₄ alkyl optionally substituted with 1-3 F, or C₁₋₄ alkoxy optionally substituted with 1-3 F;
or one instance of R⁶ and L¹, together with the intervening atoms, are joined to form an optionally substituted 5-7 membered ring structure.

65. The compound of claim 64, or a pharmaceutically acceptable salt thereof, wherein L¹ is O.

66. The compound of claim 64 or 65, or a pharmaceutically acceptable salt thereof, wherein h is 0.

67. The compound of claim 64 or 65, or a pharmaceutically acceptable salt thereof, wherein h is 1.

68. The compound of claim 67, or a pharmaceutically acceptable salt thereof, wherein R⁶ is F, Cl, C₁₋₄ alkyl optionally substituted with 1-3 F, or cyclopropyl.

69. The compound of any one of claims 1-68, or a pharmaceutically acceptable salt thereof, wherein X is C(O).

70. The compound of any one of claims 1-68, or a pharmaceutically acceptable salt thereof, wherein X is 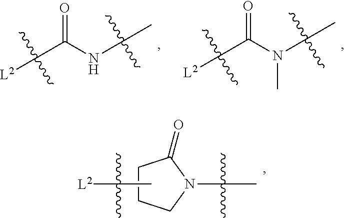
or S(O)₂.

71. The compound of any one of claims 1-70, or a pharmaceutically acceptable salt thereof, wherein ring A is an optionally substituted 4-7 membered monocyclic heterocyclyl having 1 or 2 ring heteroatoms, each independently selected from N, O, and S, preferably, at least one of the ring heteroatoms is N.

72. The compound of claim 71, or a pharmaceutically acceptable salt thereof, wherein when substituted, the 4-7 membered monocyclic heterocyclyl is substituted with 1-5 (e.g., 1, 2, or 3) G^{A}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C}; wherein G^{C} at each occurrence is independently F, OH, C₁₋₄ alkyl optionally substituted with 1-3 F, or C₁₋₄ alkoxy optionally substituted with 1-3 F.

73. The compound of any one of claims 1-70, or a pharmaceutically acceptable salt thereof, wherein ring A is an optionally substituted 6-10 membered fused, spiro, or bridged bicyclic heterocyclyl having 1 or 2 ring heteroatoms, each independently selected from N, O, and S, provided at least one of the ring heteroatoms is N.

74. The compound of claim 73, or a pharmaceutically acceptable salt thereof, wherein when substituted, the 6-10 membered fused, spiro, or bridged bicyclic heterocyclyl is substituted with 1-5 (e.g., 1, 2, or 3) G^{A}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁₋₄ alkyl) (C₁₋₄ alkyl); or C₁₋₄ alkoxy optionally substituted with 1-5 (e.g., 1, 2, or 3) G^{C}; wherein G^{C} at each occurrence is independently F, OH, C₁₋₄ alkyl optionally substituted with 1-3 F, or C₁₋₄ alkoxy optionally substituted with 1-3 F.

75. The compound of any one of claims 1-70, or a pharmaceutically acceptable salt thereof, wherein ring A is 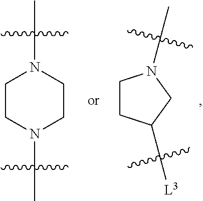
each of which is optionally substituted.

76. The compound of claim 75, or a pharmaceutically acceptable salt thereof, wherein when substituted, the piperazine or pyrrolidine is substituted with 1-5 (e.g., 1, 2, or 3) G^{A}, wherein G^{A} at each occurrence is independently halo (preferably, F) or CN; oxo; C₁₋₄ alkyl optionally substituted with 1-5 (e.g., 1, 2, or 3) G^{C}; OH; NH₂; NH (C₁₋₄ alkyl); N(C₁-4 alkyl) (C₁₋₄ alkyl); or C₁₋₄ alkoxy optionally substituted with 1-5 (e.g., 1, 2, or 3) G^{C};
wherein G^{C} at each occurrence is independently F, OH, C₁₋₄ alkyl optionally substituted with 1-3 F, or C₁₋₄ alkoxy optionally substituted with 1-3 F,
or two substituents of the piperazine or pyrrolidine, together with the intervening atom(s), are joined to form a 3-4 membered ring, such as cyclopropyl, and the piperazine or pyrrolidine is optionally further substituted with 1-3 G^{A}, wherein G^{A} is defined above, e.g., ring A can be 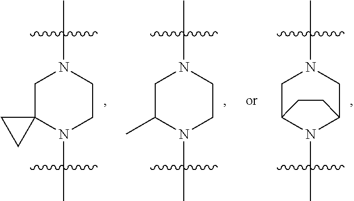
wherein either the top or the bottom attaching point can be connected to L³-Ring B, preferably, the bottom attaching point is connected to L³-Ring B.

77. The compound of any one of claims 1-70, or a pharmaceutically acceptable salt thereof, wherein ring A is

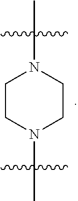 [SMILES: *N1CCN(*)CC1]

78. The compound of any one of claims 1-77, or a pharmaceutically acceptable salt thereof, wherein L³ is null.

79. The compound of any one of claims 1-77, or a pharmaceutically acceptable salt thereof, wherein L³ is O, NH, or N(C₁₋₄ alkyl), provided that L³ does not connect to a ring heteroatom of ring A.

80. The compound of any one of claims 1-79, or a pharmaceutically acceptable salt thereof, wherein ring B is an optionally substituted 5 or 6 membered heteroaryl having 1-3 ring heteroatoms, each independently selected from N, O, and S.

81. The compound of claim 80, or a pharmaceutically acceptable salt thereof, wherein the 5 or 6 membered heteroaryl is pyridine, pyrazine, thiazole, thiadiazole, or pyrimidine.

82. The compound of claim 80 or 81, or a pharmaceutically acceptable salt thereof, wherein when substituted, the 5 or 6 membered heteroaryl is substituted with 1-3 substituents independently selected from F, Cl, Br, CN, C₁₋₄ alkyl optionally substituted with 1-3 F, OH, cyclopropyl, cyclobutyl, or C₁₋₄ alkoxy optionally substituted with 1-3 F, or the 5 or 6 membered heteroaryl is substituted with 1-3 substituents (preferably 1) independently selected from (1) F, Cl, Br, OH, or CN, (2) C₁₋₄ alkyl optionally substituted with 1-3 F, (3) hydroxyl substituted C₁₋₄ alkyl, (4) cyclopropyl or cyclobutyl, each optionally substituted 1 or 2 substituents independently F, CN, or OH, (5) C₂₋₄ alkynyl optionally substituted with 1-3 F; or (6) C₁₋₄ heteroalkyl having 1 or 2 heteroatoms independently selected from O and N, which is optionally substituted with 1-3 F.

83. The compound of claim 80 or 81, or a pharmaceutically acceptable salt thereof, wherein when substituted, the 5 or 6 membered heteroaryl is substituted with 1 or 2 substituents, preferably one substituent, independently selected from F, Cl, CN, C₁₋₄ alkyl optionally substituted with 1-3 F (e.g., CHF₂ or CF₃), or cyclopropyl.

84. The compound of any one of claims 1-79, or a pharmaceutically acceptable salt thereof, wherein ring B is 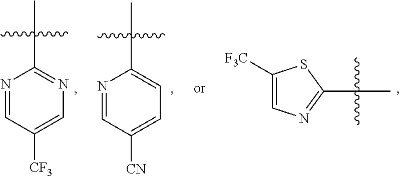
or ring B is 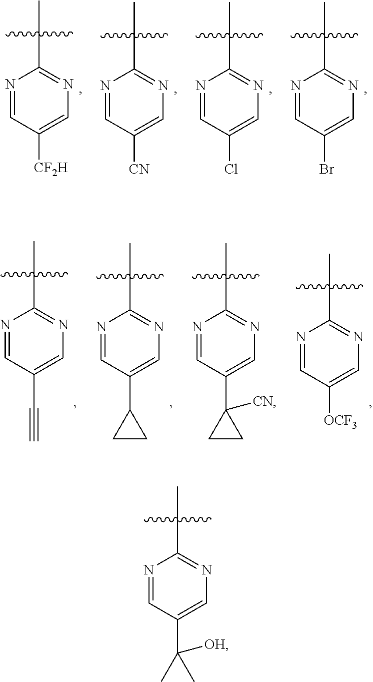
or ring B is

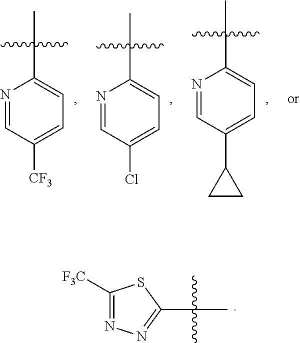

85. The compound of any one of claims 1-77, or a pharmaceutically acceptable salt thereof, wherein L³ is null, and as applicable, ring A and ring B together represent an optionally substituted cyclic structure, such as an optionally substituted piperidine, piperazine, or a fused tetrahydro triazolopyrimidine ring, e.g.,

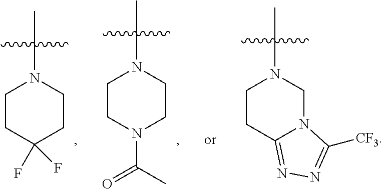

86. A compound selected from Compound Nos. 1-353, or compounds described in Table A herein, or a pharmaceutically acceptable salt thereof.

87. A pharmaceutical composition comprising the compound according to any one of claims 1-86, or a pharmaceutically acceptable salt thereof, and a pharmaceutically acceptable excipient.

88. A method of treating cancer in a subject in need thereof, comprising administering to the subject a therapeutically effective amount of the compound according to any one of claims 1-86 or a pharmaceutically acceptable salt thereof, or the pharmaceutical composition of claim 87.

89. The method of claim 88, wherein the cancer is breast cancer, cancer of the central nervous system, endometrium cancer, kidney cancer, large intestine cancer, lung cancer, esophagus cancer, ovarian cancer, pancreatic cancer, prostate cancer, stomach cancer, head and neck cancer (upper aerodigestive cancer), urinary tract cancer, or colon cancer.
